(19) United States
(12) Patent Application Publication
(10) Pub. No.: US 2024/0336601 A1
(43) Pub. Date: Oct. 10, 2024

(54) 4-PHENYL-INDOLE DERIVATIVES AND RELATED USES

(71) Applicant: Chulalongkorn University, Bangkok (TH)

(72) Inventors: Jakapun SOPONPONG, Bangkok (TH); Tanachote RUENGSATRA, Bangkok (TH); Nonthaneth NALINRATANA, Bangkok (TH); Udomsak UDOMNILOBOL, Bangkok (TH); Supanan AMPAWA, Bangkok (TH); Nopparat THAVORNSIN, Bangkok (TH); Jakkrit SRISA, Bangkok (TH); Eakkaphon RATTANANGKOOL, Bangkok (TH); Chayan CHAROENPAKDEE, Bangkok (TH); Pongkorn CHAIYAKUNVAT, Bangkok (TH); Sirikan DEESIRI, Bangkok (TH); Songkiat SONGTHAMMANUPHAP, Bangkok (TH); Wilasinee DUNKOKSUNG, Bangkok (TH); Tirayut VILAIVAN, Bangkok (TH); Supranee BURANAPRADITKUN, Bangkok (TH); Trairak PISITKUN, Bangkok (TH); Nattiya HIRANKARN, Bangkok (TH); Thomayant PRUEKSARITANONT, Bangkok (TH)

(21) Appl. No.: 18/259,408

(22) PCT Filed: Dec. 30, 2021
(86) PCT No.: PCT/US2021/065754

§ 371 Date: Jun. 27, 2023

Related U.S. Application Data
(60) Provisional application No. 63/218,135, filed on Jul. 2, 2021.
(60) Provisional application No. 63/132,020, filed on Dec. 30, 2020.

(51) Int. Cl. C07D 405/10; A61K 31/397; A61K 31/404; A61K 31/416; A61K 31/4178; A61K 31/437; A61K 31/4439; A61K 31/497; A61K 31/546; A61P 35/00; C07D 209/08; C07D 401/14; C07D 403/14; C07D 405/14; C07D 487/04; C07D 487/10; C07D 513/04; C07D 519/00

(52) U.S. Cl. CPC C07D 405/10; A61K 31/397; A61K 31/404; A61K 31/416; A61K 31/4178; A61K 31/437; A61K 31/4439; A61K 31/497; A61K 31/546; A61P 35/00; C07D 209/08; C07D 401/14; C07D 403/14; C07D 405/14; C07D 487/04; C07D 487/10; C07D 513/04; C07D 519/00

(57) ABSTRACT

The present disclosure relates to compounds of Formula (I):

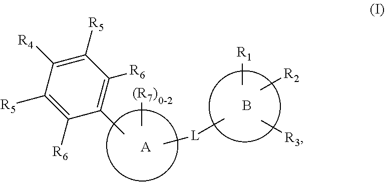

and to their prodrugs, pharmaceutically acceptable salts, pharmaceutical compositions, methods of use, and methods for their preparation. The compounds disclosed herein are useful for modulating PD-1 activity, PD-L1 activity, and/or the PD-1/PD-L1 interaction and may be used in the treatment of disorders in which PD-1 activity, PD-L1 activity, and/or PD-1/PD-L1 interaction is implicated, such as cancer.

### RELATED APPLICATIONS

This application claims priority to, and the benefit of, U.S. Provisional Application Nos. 63/132,020, filed Dec. 30, 2020, and 63/218,135, filed Jul. 2, 2021, the entire contents of each of which are incorporated herein by reference.

### BACKGROUND

The present disclosure relates to small molecules useful as inhibitors of PD-1 activity, PD-L1 activity, or the PD-1/PD-L1 interaction.

Programmed death-1 (CD279) is a receptor on T cells that has been shown to suppress activating signals from the T cell receptor when bound by either of its ligands, Programmed death-ligand 1 (PD-L1, CD274, B7-Hl) or Programmed death-ligand 2 (PD-L2) (CD273, B7-DC). When Programmed death-1 (PD-1) expressing T cells contact cells expressing its ligands, functional activities in response to antigenic stimuli, including proliferation, cytokine secretion, and cytotoxicity are reduced. PD-1/PD-Ligand interactions down regulate immune responses during resolution of an infection or tumor, or during the development of self-tolerance. Chronic antigen stimulation, such as that which occurs during tumor disease or chronic infections, results in T cells that express elevated levels of PD-1 and are dysfunctional with respect to activity towards the chronic antigen. This is termed “T cell exhaustion.” B cells also display PD-1/PD-ligand suppression and “exhaustion.”

Blockade of the PD-1/PD-L1 ligation using antibodies to PD-L1 has been shown to restore and augment T cell activation in many systems. Patients with advanced cancer benefit from therapy with a monoclonal antibody to PD-L1. Preclinical animal models of tumors and chronic infections have shown that blockade of the PD-1/PD-L1 pathway by monoclonal antibodies can enhance the immune response and result in tumor rejection or control of infection. Antitumor immunotherapy via PD-1/PD-L1 blockade may augment therapeutic immune response to a number of histologically distinct tumors.

Interference with the PD-1/PD-L1 interaction has also shown enhanced T cell activity in chronic infection systems. Chronic lymphocytic chorio meningitis virus infection of mice also exhibits improved virus clearance and restored immunity with blockade of PD-L1. Humanized mice infected with HIV-1 show enhanced protection against viremia and viral depletion of CD4+ T cells. Blockade of PD-1/PD-L1 through monoclonal antibodies to PD-L1 can restore in vitro antigen-specific functionality to T cells from HIV patients, HCV patients or HBV patients.

The disclosure arises from a need to provide further compounds for blocking or inhibiting block or inhibit PD-1, PD-L1 and/or the PD-1/PD-L1 which may be useful for treating cancer. In particular, compounds with improved physicochemical, pharmacological and pharmaceutical properties to existing compounds are desirable.

### SUMMARY

In some aspects, the present disclosure provides, inter alia, a compound of Formula (I):

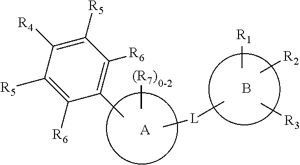

or a prodrug, solvate, or pharmaceutically acceptable salt thereof, wherein:
Ring A is 7- to 10-membered heterocyclyl or 7- to 10-membered heteroaryl;
Ring B is C₃-C₁₀ cycloalkyl, 3- to 10-membered heterocyclyl, C₆-C₁₀ aryl, or 5- to 10-membered heteroaryl;
L is absent, —C(O)—, or —CH₂—;
each R₁ and R₃ is independently H, halogen, —CN, C₁-C₆ alkyl, C₂-C₆ alkenyl, C₂-C₆ alkynyl, C₁-C₆ haloalkyl, —O—C₁-C₆ alkyl, —NH—C₁-C₆ alkyl, or C₃-C₇ cycloalkyl, wherein the alkyl, alkenyl, alkynyl, or cycloalkyl is optionally substituted with one or more R₁ₐ;
R₁ₐ is C₆-C₁₀ aryl optionally substituted with one or more halogen, —CN, or —OH;
R₂ is H, —(CH₂)ₙ—N(R₂ₐ)(R_{2b}), —(CH₂)ₙ—OH, —(CH₂)ₙ—O(C₁-C₆ alkyl), —(CH₂)ₙ—O(C₆-C₁₀ aryl), C₃-C₇ cycloalkyl, 3- to 10-membered heterocyclyl, C₆-C₁₀ aryl, or 5- to 10-membered heteroaryl, wherein the cycloalkyl, heterocyclyl, aryl, or heteroaryl is optionally substituted with one or more R_{2b};
R₂ₐ is H or C₁-C₆ alkyl;
R_{2b} is C₁-C₆ alkyl, C₂-C₆ alkenyl, C₂-C₆ alkynyl, C₁-C₆ haloalkyl, or C₃-C₇ cycloalkyl, wherein the alkyl, alkenyl, alkynyl, or cycloalkyl is optionally substituted with one or more R_{2b′}, or R_{2b} is 3- to 10-membered heterocyclyl optionally substituted with one or more oxo or —(C₁-C₆ alkyl)-OH,
or R₂, and R_{2b} come together to form a 3- to 10-membered heterocyclyl optionally substituted with one or more R_{2b′};
R_{2b′} is oxo, halogen, C₁-C₆ alkyl, C₂-C₆ alkenyl, C₂-C₆ alkynyl, C₁-C₆ haloalkyl, —(CH₂)ₘ—OR_{2b″}, —(CH₂)ₘ—N(R_{2b′″})(R_{2b″″}), —(CH₂)ₘ—C(O)OR_{2b′″}, —C(O)R_{2b′}, —C(O)N(R_{2b′})(R_{2b′″}), —N(R_{2b′})C(O)R_{2b′}, C₃-C₇ cycloalkyl, 3- to 10-membered heterocyclyl, or 5- to 10-membered heteroaryl, wherein the alkyl, alkenyl, alkynyl, cycloalkyl, or heterocyclyl is optionally substituted with one or more R_{2b″};
R_{2b″} is H, oxo, —CN, —OH, C₁-C₆ alkyl, C₂-C₆ alkenyl, C₂-C₆ alkynyl, or C₁-C₆ haloalkyl, wherein the alkyl, alkenyl, or alkynyl is optionally substituted with 3- to 10-membered heterocyclyl, wherein the heterocyclyl is optionally substituted with oxo;
R_{2b″} is H, C₁-C₆ alkyl optionally substituted with one or more —C(O)OH, —OH or —NH₂, C₂-C₆ alkenyl, C₂-C₆ alkynyl, C₃-C₇ cycloalkyl, or —C(O)C₁-C₆ alkyl;
each R₄ or R₅ is independently H, —O—C₁-C₆ alkyl, —NH—C₁-C₆ alkyl, —NH-(5- to 10-membered heteroaryl), —NH—(C₆-C₁₀ aryl), —NH—C(O)R₅ₐ, C₁-C₆ alkyl, C₂-C₆ alkenyl, C₂-C₆ alkynyl, C₁-C₆ haloalkyl, C₆-C₁₀ aryl, or 5- to 10-membered heteroaryl, wherein the alkyl, alkenyl, alkynyl, aryl, or heteroaryl is optionally substituted with one or more R₅ₐ₁, or R₄ and R₅ come together to form a C₄-C₇ cycloalkyl or 4- to 10-membered heterocyclyl;
R₅ₐ is C₃-C₇ cycloalkyl, 3- to 10-membered heterocyclyl, C₆-C₁₀ aryl, or 5- to 10-membered heteroaryl, wherein the cycloalkyl, heterocyclyl, aryl, or heteroaryl is optionally substituted with one or more R₅ₐ₁;
R₅ₐ₁ is halogen, —CN, —(CH₂)ₚ—N(R_{5a1′})(R_{5b1′}), —O—C₁-C₆alkyl, —(CH₂)ₚ—C₃-C₇ cycloalkyl wherein the cycloalkyl is optionally substituted with one or more —C(O)OH, —OH or —NH₂, C₁-C₆ alkyl optionally substituted with one or more halogen, —C(O)OH, —OH, or —NH₂, or 3- to 10-membered heterocyclyl optionally substituted with one or more —(C₁-C₆ alkyl)-OH;
R_{5b1′} is H or C₁-C₆ alkyl;
R_{5a1′} is H, C₁-C₆ alkyl, C₂-C₆ alkenyl, C₂-C₆ alkynyl, C₁-C₆ haloalkyl, C₃-C₈ cycloalkyl, 3- to 10-membered heterocyclyl, C₆-C₁₀ aryl, or 5- to 10-membered heteroaryl, wherein the alkyl, alkenyl, alkynyl, cycloalkyl, heterocyclyl, aryl, or heteroaryl is optionally substituted with one or more R_{5a1″},
or R_{5a1′} and R_{5b1′} come together to form a 3- to 10-membered heterocyclyl optionally substituted with one or more R_{5a1″};
R_{5a1″} is oxo, —CN, —OH, C₁-C₆ alkyl optionally substituted with one or more —C(O)OH, —OH or —NH₂, C₂-C₆ alkenyl, C₂-C₆ alkynyl, C₁-C₆ haloalkyl, —C(O)OH, —C(O)C₁-C₆ alkyl, —C(O)O—C₁-C₆ alkyl, —C(O)NH₂, —C(O)NH—C₁-C₆ alkyl, —C(O)NH—C₃-C₁₀ cycloalkyl, —NH—C₁-C₆ alkyl-R_{5a1′″}, —(CH₂)_{q}—(C₃-C₇ cycloalkyl), or —(CH₂)_{q}-(3- to 10-membered heterocyclyl), wherein the cycloalkyl or heterocyclyl is optionally substituted with one or more oxo, C₁-C₆ alkyl, C₂-C₆ alkenyl, C₂-C₆ alkynyl, —C(O)OH, or —OH;
R_{5a1′″} is 3- to 10-membered heterocyclyl optionally substituted with oxo;
each R₆ is independently H, halogen, —CN, C₁-C₆ alkyl, C₂-C₆ alkenyl, C₂-C₆ alkynyl, C₁-C₆ haloalkyl, —O—C₁-C₆alkyl, —NH—C₁-C₆ alkyl, or C₃-C₇ cycloalkyl;
R₇ is C₁-C₆ alkyl, C₂-C₆ alkenyl, C₂-C₆ alkynyl, or C₁-C₆ haloalkyl;
m is 0, 1, 2, 3, or 4;
n is 0, 1, 2, 3, or 4;
p is 0, 1, 2, 3, or 4; and
q is 0, 1, 2, 3, or 4,
wherein at least one of R₄, R₅, or R₆ is not H; and
provided that:
(i) when Ring B is an optionally substituted 5-membered heteroaryl or 9-membered heterocyclyl and R_{5a1′} and R_{5b1′} come together to form a 5-membered heterocyclyl substituted with one R_{5a1″}, then R_{5a1″} is not —OH; or
(ii) when Ring B is an optionally substituted 9-membered heterocyclyl and R_{5a1″} and R_{5b1′}, come together to form a 6-membered heterocyclyl, then R₂ is not —(CH₂)₂—OH; or

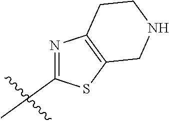 [SMILES: *c1nc2c(s1)CNCC2]

(iv) when R₅ is —NH—C(O)R₅ₐ, R₅ₐ is not a substituted 5-membered heteroaryl.

In some aspects, the present disclosure provides, inter alia, a compound of Formula (I):

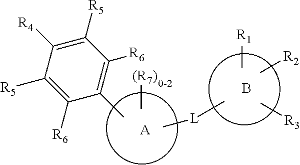

or a prodrug, solvate, or pharmaceutically acceptable salt thereof, wherein:
Ring A is 7- to 10-membered heterocyclyl or 7- to 10-membered heteroaryl;
Ring B is C₃-C₁₀ cycloalkyl, 3- to 10-membered heterocyclyl, C₆-C₁₀ aryl, or 5- to 10-membered heteroaryl;
L is absent, —C(O)—, or —CH₂—;
each R₁ and R₃ is independently H, halogen, —CN, C₁-C₆ alkyl, C₂-C₆ alkenyl, C₂-C₆ alkynyl, C₁-C₆ haloalkyl, —O—C₁-C₆ alkyl, —NH—C₁-C₆ alkyl, or C₃-C₇ cycloalkyl, wherein the alkyl, alkenyl, alkynyl, or cycloalkyl is optionally substituted with one or more R₁ₐ;
R₁ₐ is C₆-C₁₀ aryl optionally substituted with one or more halogen, —CN, or —OH;
R₂ is H, —(CH₂)ₙ—N(R₂ₐ)(R_{2b}), —(CH₂)ₙ—OH, —(CH₂)ₙ—O(C₁-C₆ alkyl), —(CH₂)ₙ—O(C₆-C₁₀ aryl), C₃-C₇ cycloalkyl, 3- to 10-membered heterocyclyl, C₆-C₁₀ aryl, or 5- to 10-membered heteroaryl, wherein the cycloalkyl, heterocyclyl, aryl, or heteroaryl is optionally substituted with one or more R_{2b′};
R_{2b} is H;
R_{2b} is C₁-C₆ alkyl, C₂-C₆ alkenyl, C₂-C₆ alkynyl, C₁-C₆ haloalkyl, or C₃-C₇ cycloalkyl, wherein the alkyl, alkenyl, alkynyl, or cycloalkyl is optionally substituted with one or more R_{2b′}, or R_{2b} is 3- to 10-membered heterocyclyl optionally substituted with one or more oxo or —(C₁-C₆ alkyl)-OH,
or R₂ₐ and R_{2b} come together to form a 3- to 10-membered heterocyclyl optionally substituted with one or more R_{2b′};
R_{2b′} is oxo, halogen, C₁-C₆ alkyl, C₂-C₆ alkenyl, C₂-C₆ alkynyl, C₁-C₆ haloalkyl, —(CH₂)ₘ—OR_{2b″}, —(CH₂)ₘ—N(R_{2b″})(R_{2b′″}), —(CH₂)ₙ—C(O)OR_{2b″}, —C(O)R_{2b′″}, —C(O)N(R_{2b′})(R_{2b′″}), —N(R_{2b″})C(O)R_{2b′″}, C₃-C₇ cycloalkyl, 3- to 10-membered heterocyclyl, or 5- to 10-membered heteroaryl, wherein the alkyl, alkenyl, alkynyl, cycloalkyl, or heterocyclyl is optionally substituted with one or more R_{2b′″};
R_{2b″} is H, oxo, —CN, —OH, C₁-C₆ alkyl, C₂-C₆ alkenyl, C₂-C₆ alkynyl, or C₁-C₆ haloalkyl, wherein the alkyl, alkenyl, or alkynyl is optionally substituted with 3- to 10-membered heterocyclyl, wherein the heterocyclyl is optionally substituted with oxo;
R_{2b′″} is H, C₁-C₆ alkyl optionally substituted with one or more —C(O)OH, —OH or —NH₂, C₂-C₆ alkenyl, C₂-C₆ alkynyl, C₃-C₇ cycloalkyl, or —C(O)C₁-C₆ alkyl;
each R₄ or R₅ is independently H, —O—C₁-C₆alkyl, —NH—C₁-C₆alkyl, —NH-(5- to 10-membered heteroaryl), —NH—(C₆-C₁₀ aryl), —NH—C(O)R₅ₐ, C₁-C₆ alkyl, C₂-C₆ alkenyl, C₂-C₆ alkynyl, C₁-C₆ haloalkyl, C₆-C₁₀ aryl, or 5- to 10-membered heteroaryl, wherein the alkyl, alkenyl, alkynyl, aryl, or heteroaryl is optionally substituted with one or more R₅ₐ₁,
or R₄ and R₅ come together to form a C₄-C₇ cycloalkyl or 4- to 10-membered heterocyclyl;
R₅ₐ is C₃-C₇ cycloalkyl, 3- to 10-membered heterocyclyl, C₆-C₁₀ aryl, or 5- to 10-membered heteroaryl, wherein the cycloalkyl, heterocyclyl, aryl, or heteroaryl is optionally substituted with one or more R₅ₐ₁;
R₅ₐ₁ is halogen, —CN, —(CH₂)ₚ—N(R_{5a1′})(R_{5b1′}), —O—C₁-C₆ alkyl, —(CH₂)ₚ—C₃-C₇ cycloalkyl wherein the cycloalkyl is optionally substituted with one or more —C(O)OH, —OH or —NH₂, C₁-C₆ alkyl optionally substituted with one or more halogen, —C(O)OH, —OH, or —NH₂, or 3- to 10-membered heterocyclyl optionally substituted with one or more —(C₁-C₆ alkyl)-OH;
R_{5b1′} is H;
R_{5a1′} is H, C₁-C₆ alkyl, C₂-C₆ alkenyl, C₂-C₆ alkynyl, C₁-C₆ haloalkyl, C₃-C₇ cycloalkyl, 3- to 10-membered heterocyclyl, C₆-C₁₀ aryl, or 5- to 10-membered heteroaryl, wherein the alkyl, alkenyl, alkynyl, cycloalkyl, heterocyclyl, aryl, or heteroaryl is optionally substituted with one or more R_{5a1″},
or R_{5a1′} and R_{5b1′} come together to form a 3- to 10-membered heterocyclyl optionally substituted with one or more R_{5a1″};
R_{5a1″} is oxo, —CN, —OH, C₁-C₆ alkyl optionally substituted with one or more —C(O)OH, —OH or —NH₂, C₂-C₆ alkenyl, C₂-C₆ alkynyl, C₁-C₆ haloalkyl, —C(O)OH, —C(O)C₁-C₆ alkyl, —C(O)O—C₁-C₆ alkyl, —C(O)NH₂, —C(O)NH—C₁-C₆ alkyl, —C(O)NH—C₃-C₁₀ cycloalkyl, —NH—C₁-C₆ alkyl-R_{5a1′″}, —(CH₂)_{q}—(C₃-C₇ cycloalkyl), or —(CH₂)_{q}-(3- to 10-membered heterocyclyl), wherein the cycloalkyl or heterocyclyl is optionally substituted with one or more oxo, C₁-C₆ alkyl, C₂-C₆ alkenyl, C₂-C₆ alkynyl, —C(O)OH, or —OH;
R_{5a1′″} is 3- to 10-membered heterocyclyl optionally substituted with oxo;
each R₆ is independently H, halogen, —CN, C₁-C₆ alkyl, C₂-C₆ alkenyl, C₂-C₆ alkynyl, C₁-C₆ haloalkyl, —O—C₁-C₆ alkyl, —NH—C₁-C₆alkyl, or C₃-C₇ cycloalkyl;
R₇ is C₁-C₆ alkyl, C₂-C₆ alkenyl, C₂-C₆ alkynyl, or C₁-C₆ haloalkyl;
m is 0, 1, 2, 3, or 4;
n is 0, 1, 2, 3, or 4;
p is 0, 1, 2, 3, or 4; and
q is 0, 1, 2, 3, or 4,
wherein at least one of R₄, R₅, or R₆ is not H; and
provided that:
(i) when Ring B is an optionally substituted 5-membered heteroaryl or 9-membered heterocyclyl and R_{5a1′} and R_{5b1′} come together to form a 5-membered heterocyclyl substituted with one R_{5a1″}, then R_{5a1″} is not —OH; or
(ii) when Ring B is an optionally substituted 9-membered heterocyclyl and R_{5a1′} and R_{5b1′} come together to form a 6-membered heterocyclyl, then R₂ is not —(CH₂)₂—OH; or

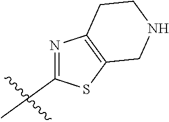 [SMILES: *c1nc2c(s1)CNCC2]

(iv) when R₅ is —NH—C(O)R₅ₐ, R₅ₐ is not a substituted 5-membered heteroaryl.

In some aspects, the present disclosure provides, inter alia, a compound of Formula (I):

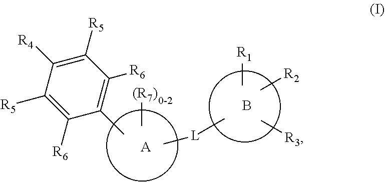

or a prodrug, solvate, or pharmaceutically acceptable salt thereof, wherein:
Ring A is 7- to 10-membered heterocyclyl or 7- to 10-membered heteroaryl;
Ring B is C₃-C₇ cycloalkyl, 3- to 10-membered heterocyclyl, C₆-C₁₀ aryl, or 5- to 10-membered heteroaryl;
L is absent, —C(O)—, or —CH₂—;
each R₁ and R₃ is independently H, halogen, —CN, C₁-C₆ alkyl, C₂-C₆ alkenyl, C₂-C₆ alkynyl, C₁-C₆ haloalkyl, —O—C₁-C₆ alkyl, —NH—C₁-C₆ alkyl, or C₃-C₇ cycloalkyl, wherein the alkyl, alkenyl, alkynyl, or cycloalkyl is optionally substituted with one or more R₁ₐ;
R₁ₐ is C₆-C₁₀ aryl optionally substituted with one or more halogen, —CN, or —OH;
R₂ is H, —(CH₂)ₙ—N(R_{2b})(R_{2b}), —(CH₂)ₙ—OH, —(CH₂)ₙ—O(C₁-C₆ alkyl), C₃-C₇ cycloalkyl, 3- to 10-membered heterocyclyl, C₆-C₁₀ aryl, or 5- to 10-membered heteroaryl, wherein the cycloalkyl, heterocyclyl, aryl, or heteroaryl is optionally substituted with one or more R_{2b′};
R₂ₐ is H;
R_{2b} is C₁-C₆ alkyl, C₂-C₆ alkenyl, C₂-C₆ alkynyl, C₁-C₆ haloalkyl, or C₃-C₇ cycloalkyl, wherein the alkyl, alkenyl, alkynyl, or cycloalkyl is optionally substituted with one or more R_{2b′}, or R_{2b} is 3- to 10-membered heterocyclyl optionally substituted with one or more oxo or —(C₁-C₆ alkyl)-OH,
or R₂ₐ and R_{2b} come together to form a 3- to 10-membered heterocyclyl optionally substituted with one or more R_{2b′};
R_{2b′} is oxo, C₁-C₆ alkyl, C₂-C₆ alkenyl, C₂-C₆ alkynyl, C₁-C₆ haloalkyl, —(CH₂)ₘ—OR_{2b′}, —(CH₂)ₘ—N(R_{2b″})(R_{2b′″}), —C(O)OR_{2b″}, —C(O)R_{2b″}, —C(O)N(R_{2b″})(R_{2b′″}), —N(R_{2b″})C(O)R_{2b′″}, C₃-C₇ cycloalkyl, or 3- to 10-membered heterocyclyl, wherein the alkyl, alkenyl, alkynyl, cycloalkyl, or heterocyclyl is optionally substituted with one or more R_{2b″};
R_{2b″} is H, oxo, —CN, —OH, C₁-C₆ alkyl, C₂-C₆ alkenyl, C₂-C₆ alkynyl, or C₁-C₆ haloalkyl, wherein the alkyl, alkenyl, or alkynyl is optionally substituted with 3- to 10-membered heterocyclyl, wherein the heterocyclyl is optionally substituted with oxo;
R_{2b′″} is H, C₁-C₆ alkyl, C₂-C₆ alkenyl, C₂-C₆ alkynyl, C₃-C₇ cycloalkyl, or —C(O)C₁-C₆ alkyl,
each R₄ or R₅ is independently H, —O—C₁-C₆alkyl, —NH—C₁-C₆alkyl, —NH-(5- to 10-membered heteroaryl), —NH—(C₆-C₁₀ aryl), —NH—C(O)R₅ₐ, C₁-C₆ alkyl, C₂-C₆ alkenyl, C₂-C₆ alkynyl, C₁-C₆ haloalkyl, C₆-C₁₀ aryl, or 5- to 10-membered heteroaryl, wherein the alkyl, alkenyl, alkynyl, aryl, or heteroaryl is optionally substituted with one or more R₅ₐ₁,
or R₄ and R₅ come together to form a C₄-C₇ cycloalkyl or 4- to 10-membered heterocyclyl;
R₅ₐ is C₃-C₇ cycloalkyl, 3- to 10-membered heterocyclyl, C₆-C₁₀ aryl, or 5- to 10-membered heteroaryl, wherein the cycloalkyl, heterocyclyl, aryl, or heteroaryl is optionally substituted with one or more R₅ₐ₁;
R₅ₐ₁ is halogen, —CN, —(CH₂)ₚ—N(R_{5a1′})(R_{5b1′}), —O—C₁-C₆alkyl, C₃-C₇ cycloalkyl, C₁-C₆ alkyl optionally substituted with one or more —OH or —NH₂, or 3- to 10-membered heterocyclyl optionally substituted with one or more —(C₁-C₆ alkyl)-OH;
R_{5b1′} is H;
R_{5a1′} is H, C₁-C₆ alkyl, C₂-C₆ alkenyl, C₂-C₆ alkynyl, C₁-C₆ haloalkyl, C₃-C₇ cycloalkyl, 3- to 10-membered heterocyclyl, C₆-C₁₀ aryl, or 5- to 10-membered heteroaryl, wherein the alkyl, alkenyl, alkynyl, cycloalkyl, heterocyclyl, aryl, or heteroaryl is optionally substituted with one or more R_{5a1″},
or R_{5a1′} and R_{5b1′} come together to form a 3- to 10-membered heterocyclyl optionally substituted with one or more R_{5a1″};
R_{5a1′} is oxo, —CN, —OH, C₁-C₆ alkyl, C₂-C₆ alkenyl, C₂-C₆ alkynyl, C₁-C₆ haloalkyl, —C(O)OH, —C(O)C₁-C₆ alkyl, —C(O)O—C₁-C₆ alkyl, —C(O)NH—C₁-C₆ alkyl, —C(O)NH—C₃-C₁₀ cycloalkyl, —NH—C₁-C₆ alkyl-R_{5a1′″}, —(CH₂)_{q}—(C₃-C₇ cycloalkyl), or —(CH₂)_{q}-(3- to 10-membered heterocyclyl), wherein the cycloalkyl or heterocyclyl is optionally substituted with one or more oxo, C₁-C₆ alkyl, C₂-C₆ alkenyl, C₂-C₆ alkynyl, —C(O)OH, or —OH;
R_{5a1′″} is 3- to 10-membered heterocyclyl optionally substituted with oxo;
each R₆ is independently H, halogen, —CN, C₁-C₆ alkyl, C₂-C₆ alkenyl, C₂-C₆ alkynyl, C₁-C₆ haloalkyl, —O—C₁-C₆alkyl, —NH—C₁-C₆alkyl, or C₃-C₇ cycloalkyl;
R₇ is C₁-C₆ alkyl, C₂-C₆ alkenyl, C₂-C₆ alkynyl, or C₁-C₆ haloalkyl;
m is 0, 1, 2, 3, or 4;
n is 0, 1, 2, 3, or 4;
p is 0, 1, 2, 3, or 4; and
q is 0, 1, 2, 3, or 4,
   wherein at least one of R₄, R₅, or R₆ is not H; and
   provided that:
(i) when Ring B is an optionally substituted 5-membered heteroaryl or 9-membered heterocyclyl and R_{5a1′} and R_{5b1′} come together to form a 5-membered heterocyclyl substituted with one R_{5a1″}, then R_{5a1″} is not —OH; or
(ii) when Ring B is an optionally substituted 9-membered heterocyclyl and R_{5a1′} and R_{5b1′}, come together to form a 6-membered heterocyclyl, then R₂ is not —(CH₂)₂—OH; or
(iii) when R₄ and R₅ come together to form a 6-membered heterocyclyl and Ring B is

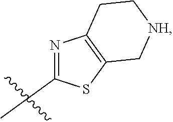 [SMILES: *c1nc2c(s1)CNCC2]

then Ring B is not substituted with —CH₃ or —CH₂CH₂—OH; or
(iv) when R₅ is —NH—C(O)R₅ₐ, R₅ₐ is not a substituted 5-membered heteroaryl.

In some aspects, the present disclosure provides a compound obtainable by, or obtained by, a method for preparing a compound as described herein (e.g., a method comprising one or more steps described in Schemes 1-6).

In some aspects, the present disclosure provides a pharmaceutical composition comprising a compound of the present disclosure, or a pharmaceutically acceptable salt thereof, and a pharmaceutically acceptable diluent or carrier.

In some aspects, the present disclosure provides an intermediate as described herein, being suitable for use in a method for preparing a compound as described herein (e.g., the intermediate is selected from the intermediates described in Examples 1-166).

In some aspects, the present disclosure provides a method of inhibiting PD-1 activity, PD-L1 activity, and/or the PD-1/PD-L1 interaction in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating or preventing a disease or disorder disclosed herein in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating a disease or disorder disclosed herein in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of inhibiting PD-1 activity, PD-L1 activity, and/or the PD-1/PD-L1 interaction in a subject in need thereof, comprising administering to the subject a compound of the present disclosure or a pharmaceutically acceptable salt thereof, or a pharmaceutical composition of the present disclosure.

In some aspects, the present disclosure provides a method of treating or preventing a disease or disorder disclosed herein in a subject in need thereof, comprising administering to the subject a compound of the present disclosure or a pharmaceutically acceptable salt thereof, or a pharmaceutical composition of the present disclosure.

In some aspects, the present disclosure provides a method of treating a disease or disorder disclosed herein in a subject in need thereof, comprising administering to the subject a compound of the present disclosure or a pharmaceutically acceptable salt thereof, or a pharmaceutical composition of the present disclosure.

In some aspects, the present disclosure provides a compound of the present disclosure or a pharmaceutically acceptable salt thereof for use in inhibiting PD-1 activity, PD-L1 activity, and/or the PD-1/PD-L1 interaction.

In some aspects, the present disclosure provides a compound of the present disclosure or a pharmaceutically acceptable salt thereof for use in treating or preventing a disease or disorder disclosed herein.

In some aspects, the present disclosure provides a compound of the present disclosure or a pharmaceutically acceptable salt thereof for use in treating a disease or disorder disclosed herein.

In some aspects, the present disclosure provides use of a compound of the present disclosure or a pharmaceutically acceptable salt thereof in the manufacture of a medicament for inhibiting PD-1 activity, PD-L1 activity, and/or the PD-1/PD-L1 interaction.

In some aspects, the present disclosure provides use of a compound of the present disclosure or a pharmaceutically acceptable salt thereof in the manufacture of a medicament for treating or preventing a disease or disorder disclosed herein.

In some aspects, the present disclosure provides use of a compound of the present disclosure or a pharmaceutically acceptable salt thereof in the manufacture of a medicament for treating a disease or disorder disclosed herein.

In some embodiments, the disease or disorder is cancer.

In some aspects, the present disclosure provides a method of preparing a compound of the present disclosure.

In some aspects, the present disclosure provides a method of preparing a compound, comprising one or more steps described herein.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present disclosure, suitable methods and materials are described below. All publications, patent applications, patents and other references mentioned herein are incorporated by reference. The references cited herein are not admitted to be prior art to the claimed invention. In the case of conflict, the present specification, including definitions, will control. In addition, the materials, methods and examples are illustrative only and are not intended to be limiting. In the case of conflict between the chemical structures and names of the compounds disclosed herein, the chemical structures will control.

Other features and advantages of the disclosure will be apparent from the following detailed description and claims.

### DETAILED DESCRIPTION

The present disclosure relates to 4-phenyl indole derivatives, prodrugs, and pharmaceutically acceptable salts thereof, which may modulate PD-1 activity, PD-L1 activity, and/or PD-1/PD-L1 interaction and are accordingly useful in methods of treatment of the human or animal body. The present disclosure also relates to processes for the preparation of these compounds, to pharmaceutical compositions comprising them and to their use in the treatment of disorders in which PD-1, PD-L1, and/or PD-1/PD-L1 is implicated, such as cancer.

### Definitions

Unless otherwise stated, the following terms used in the specification and claims have the following meanings set out below.

As used herein, “alkyl”, “C₁, C₂, C₃, C₄, C₅ or C₆ alkyl” or “C₁-C₆ alkyl” is intended to include C₁, C₂, C₃, C₄, C₅ or C₆ straight chain (linear) saturated aliphatic hydrocarbon groups and C₃, C₄, C₅ or C₆ branched saturated aliphatic hydrocarbon groups. For example, C₁-C₆ alkyl is intends to include C₁, C₂, C₃, C₄, C₅ and C₆ alkyl groups. Examples of alkyl include, moieties having from one to six carbon atoms, such as, but not limited to, methyl, ethyl, n-propyl, i-propyl, n-butyl, s-butyl, t-butyl, n-pentyl, i-pentyl, or n-hexyl. In some embodiments, a straight chain or branched alkyl has six or fewer carbon atoms (e.g., C₁-C₆ for straight chain, C₃-C₆ for branched chain), and in another embodiment, a straight chain or branched alkyl has four or fewer carbon atoms.

As used herein, the term “optionally substituted alkyl” refers to unsubstituted alkyl or alkyl having designated substituents replacing one or more hydrogen atoms on one or more carbons of the hydrocarbon backbone. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

As used herein, the term “alkenyl” includes unsaturated aliphatic groups analogous in length and possible substitution to the alkyls described above, but that contain at least one double bond.

For example, the term “alkenyl” includes straight chain alkenyl groups (e.g., ethenyl, propenyl, butenyl, pentenyl, hexenyl, heptenyl, octenyl, nonenyl, decenyl), and branched alkenyl groups. In certain embodiments, a straight chain or branched alkenyl group has six or fewer carbon atoms in its backbone (e.g., C₂-C₆ for straight chain, C₃-C₆ for branched chain). The term “C₂-C₆” includes alkenyl groups containing two to six carbon atoms. The term “C₃-C₆” includes alkenyl groups containing three to six carbon atoms.

As used herein, the term “optionally substituted alkenyl” refers to unsubstituted alkenyl or alkenyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, heterocyclyl, alkylaryl, or an aromatic or heteroaromatic moiety.

As used herein, the term “alkynyl” includes unsaturated aliphatic groups analogous in length and possible substitution to the alkyls described above, but which contain at least one triple bond.

For example, “alkynyl” includes straight chain alkynyl groups (e.g., ethynyl, propynyl, butynyl, pentynyl, hexynyl, heptynyl, octynyl, nonynyl, decynyl), and branched alkynyl groups. In certain embodiments, a straight chain or branched alkynyl group has six or fewer carbon atoms in its backbone (e.g., C₂-C₆ for straight chain, C₃-C₆ for branched chain). The term “C₂-C₆” includes alkynyl groups containing two to six carbon atoms. The term “C₃-C₆” includes alkynyl groups containing three to six carbon atoms. As used herein, “C₂-C₆ alkenylene linker” or “C₂-C₆ alkynylene linker” is intended to include C₂, C₃, C₄, C₅ or C₆ chain (linear or branched) divalent unsaturated aliphatic hydrocarbon groups. For example, C₂-C₈ alkenylene linker is intended to include C₂, C₃, C₄, C₅ and C₆ alkenylene linker groups.

As used herein, the term “optionally substituted alkynyl” refers to unsubstituted alkynyl or alkynyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

Other optionally substituted moieties (such as optionally substituted cycloalkyl, heterocycloalkyl, aryl, or heteroaryl) include both the unsubstituted moieties and the moieties having one or more of the designated substituents. For example, substituted heterocycloalkyl includes those substituted with one or more alkyl groups, such as 2,2,6,6-tetramethyl-piperidinyl and 2,2,6,6-tetramethyl-1,2,3,6-tetrahydropyridinyl.

As used herein, the term “cycloalkyl” refers to a saturated or partially unsaturated hydrocarbon monocyclic or polycyclic (e.g., fused, bridged, or spiro rings) system having 3 to 30 carbon atoms (e.g., C₃-C₁₂, C₃-C₁₀, or C₃-C₈). Examples of cycloalkyl include, but are not limited to, cyclopropyl, cyclobutyl, cyclopentyl, cyclohexyl, cycloheptyl, cyclooctyl, cyclopentenyl, cyclohexenyl, cycloheptenyl, 1,2,3,4-tetrahydronaphthalenyl, and adamantyl. In the case of polycyclic cycloalkyl, only one of the rings in the cycloalkyl needs to be non-aromatic.

As used herein, the term “heterocycloalkyl” refers to a saturated or partially unsaturated 3-8 membered monocyclic, 7-12 membered bicyclic (fused, bridged, or spiro rings), or 11-14 membered tricyclic ring system (fused, bridged, or spiro rings) having one or more heteroatoms (such as O, N, S, P, or Se), e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulfur, unless specified otherwise. Examples of heterocycloalkyl groups include, but are not limited to, piperidinyl, piperazinyl, pyrrolidinyl, dioxanyl, tetrahydrofuranyl, isoindolinyl, indolinyl, imidazolidinyl, pyrazolidinyl, oxazolidinyl, isoxazolidinyl, triazolidinyl, oxiranyl, azetidinyl, oxetanyl, thietanyl, 1,2,3,6-tetrahydropyridinyl, tetrahydropyranyl, dihydropyranyl, pyranyl, morpholinyl, tetrahydrothiopyranyl, 1,4-diazepanyl, 1,4-oxazepanyl, 2-oxa-5-azabicyclo[2.2.1]heptanyl, 2,5-diazabicyclo[2.2.1]heptanyl, 2-oxa-6-azaspiro[3.3]heptanyl, 2,6-diazaspiro[3.3]heptanyl, 1,4-dioxa-8-azaspiro[4.5]decanyl, 1,4-dioxaspiro[4.5]decanyl, 1-oxaspiro[4.5]decanyl, 1-azaspiro[4.5]decanyl, 3′H-spiro[cyclohexane-1,1′-isobenzofuran]-yl, 7′H-spiro[cyclohexane-1,5′-furo[3,4-b]pyridin]-yl, 3′H-spiro[cyclohexane-1,1′-furo[3,4-c]pyridin]-yl, 3-azabicyclo[3.1.0]hexanyl, 3-azabicyclo[3.1.0]hexan-3-yl, 1,4,5,6-tetrahydropyrrolo[3,4-c]pyrazolyl, 3,4,5,6,7,8-hexahydropyrido[4,3-d]pyrimidinyl, 4,5,6,7-tetrahydro-1H-pyrazolo[3,4-c]pyridinyl, 5,6,7,8-tetrahydropyrido[4,3-d]pyrimidinyl, 2-azaspiro[3.3]heptanyl, 2-methyl-2-azaspiro[3.3]heptanyl, 2-azaspiro[3.5]nonanyl, 2-methyl-2-azaspiro[3.5]nonanyl, 2-azaspiro[4.5]decanyl, 2-methyl-2-azaspiro[4.5]decanyl, 2-oxa-azaspiro[3.4]octanyl, 2-oxa-azaspiro[3.4]octan-6-yl, 5,6-dihydro-4H-cyclopenta[b]thiophenyl, and the like. In the case of multicyclic heterocycloalkyl, only one of the rings in the heterocycloalkyl needs to be non-aromatic (e.g., 4,5,6,7-tetrahydrobenzo[c]isoxazolyl).

As used herein, the term “aryl” includes groups with aromaticity, including “conjugated,” or multicyclic systems with one or more aromatic rings and do not contain any heteroatom in the ring structure. The term aryl includes both monovalent species and divalent species. Examples of aryl groups include, but are not limited to, phenyl, biphenyl, naphthyl and the like. Conveniently, an aryl is phenyl.

As used herein, the term “heteroaryl” is intended to include a stable 5-, 6-, or 7-membered monocyclic or 7-, 8-, 9-, 10-, 11- or 12-membered bicyclic aromatic heterocyclic ring which consists of carbon atoms and one or more heteroatoms, e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulfur. The nitrogen atom may be substituted or unsubstituted (i.e., N or NR wherein R is H or other substituents, as defined). The nitrogen and sulfur heteroatoms may optionally be oxidised (i.e., N→O and S(O)ₚ, where p=1 or 2). It is to be noted that total number of S and O atoms in the aromatic heterocycle is not more than 1. Examples of heteroaryl groups include pyrrole, furan, thiophene, thiazole, isothiazole, imidazole, triazole, tetrazole, pyrazole, oxazole, isoxazole, isothiazole, pyridine, pyrazine, pyridazine, pyrimidine, and the like. Heteroaryl groups can also be fused or bridged with alicyclic or heterocyclic rings, which are not aromatic so as to form a multicyclic system (e.g., 4,5,6,7-tetrahydrobenzo[c]isoxazolyl). In some embodiments, the heteroaryl is thiophenyl or benzothiophenyl. In some embodiments, the heteroaryl is thiophenyl. In some embodiments, the heteroaryl benzothiophenyl.

Furthermore, the terms “aryl” and “heteroaryl” include multicyclic aryl and heteroaryl groups, e.g., tricyclic, bicyclic, e.g., naphthalene, benzoxazole, benzodioxazole, benzothiazole, benzoimidazole, benzothiophene, quinoline, isoquinoline, naphthrydine, indole, benzofuran, purine, benzofuran, deazapurine, indolizine.

The cycloalkyl, heterocycloalkyl, aryl, or heteroaryl ring can be substituted at one or more ring positions (e.g., the ring-forming carbon or heteroatom such as N) with such substituents as described above, for example, alkyl, alkenyl, alkynyl, halogen, hydroxyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Aryl and heteroaryl groups can also be fused or bridged with alicyclic or heterocyclic rings, which are not aromatic so as to form a multicyclic system (e.g., tetralin, methylenedioxyphenyl such as benzo[d][1,3]dioxole-5-yl).

As used herein, the term “substituted,” means that any one or more hydrogen atoms on the designated atom is replaced with a selection from the indicated groups, provided that the designated atom's normal valency is not exceeded, and that the substitution results in a stable compound. When a substituent is oxo or keto (i.e., ═O), then 2 hydrogen atoms on the atom are replaced. Keto substituents are not present on aromatic moieties. Ring double bonds, as used herein, are double bonds that are formed between two adjacent ring atoms (e.g., C═C, C═N or N═N). “Stable compound” and “stable structure” are meant to indicate a compound that is sufficiently robust to survive isolation to a useful degree of purity from a RM, and formulation into an efficacious therapeutic agent.

When a bond to a substituent is shown to cross a bond connecting two atoms in a ring, then such substituent may be bonded to any atom in the ring. When a substituent is listed without indicating the atom via which such substituent is bonded to the rest of the compound of a given formula, then such substituent may be bonded via any atom in such formula. Combinations of substituents and/or variables are permissible, but only if such combinations result in stable compounds.

When any variable (e.g., R) occurs more than one time in any constituent or formula for a compound, its definition at each occurrence is independent of its definition at every other occurrence. Thus, for example, if a group is shown to be substituted with 0-2 R moieties, then the group may optionally be substituted with up to two R moieties and R at each occurrence is selected independently from the definition of R. Also, combinations of substituents and/or variables are permissible, but only if such combinations result in stable compounds.

As used herein, the term “hydroxy” or “hydroxyl” includes groups with an —OH or —O—.

As used herein, the term “halo” or “halogen” refers to fluoro, chloro, bromo and iodo.

The term “haloalkyl” or “haloalkoxyl” refers to an alkyl or alkoxyl substituted with one or more halogen atoms.

As used herein, the term “optionally substituted haloalkyl” refers to unsubstituted haloalkyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

As used herein, the term “alkoxy” or “alkoxyl” includes substituted and unsubstituted alkyl, alkenyl and alkynyl groups covalently linked to an oxygen atom. Examples of alkoxy groups or alkoxyl radicals include, but are not limited to, methoxy, ethoxy, isopropyloxy, propoxy, butoxy and pentoxy groups. Examples of substituted alkoxy groups include halogenated alkoxy groups.

The alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy and trichloromethoxy.

As used herein, the expressions “one or more of A, B, or C,” “one or more A, B, or C,” “one or more of A, B, and C,” “one or more A, B, and C,” “selected from the group consisting of A, B, and C”, “selected from A, B, and C”, and the like are used interchangeably and all refer to a selection from a group consisting of A, B, and/or C, i.e., one or more As, one or more Bs, one or more Cs, or any combination thereof, unless indicated otherwise.

It is to be understood that the present disclosure provides methods for the synthesis of the compounds of any of the Formulae described herein. The present disclosure also provides detailed methods for the synthesis of various disclosed compounds of the present disclosure according to the following schemes as well as those shown in the Examples.

It is to be understood that, throughout the description, where compositions are described as having, including, or comprising specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the invention remains operable. Moreover, two or more steps or actions can be conducted simultaneously.

It is to be understood that the synthetic processes of the disclosure can tolerate a wide variety of functional groups, therefore various substituted starting materials can be used. The processes generally provide the desired final compound at or near the end of the overall process, although it may be desirable in certain instances to further convert the compound to a pharmaceutically acceptable salt thereof.

It is to be understood that compounds of the present disclosure can be prepared in a variety of ways using commercially available starting materials, compounds known in the literature, or from readily prepared intermediates, by employing standard synthetic methods and procedures either known to those skilled in the art, or which will be apparent to the skilled artisan in light of the teachings herein. Standard synthetic methods and procedures for the preparation of organic molecules and functional group transformations and manipulations can be obtained from the relevant scientific literature or from standard textbooks in the field. Although not limited to any one or several sources, classic texts such as Smith, M. B., March, J., March's Advanced Organic Chemistry: Reactions, Mechanisms, and Structure, 5^{th} edition, John Wiley & Sons: New York, 2001; Greene, T. W., Wuts, P. G. M., Protective Groups in Organic Synthesis, 3^{rd} edition, John Wiley & Sons: New York, 1999; R. Larock, Comprehensive Organic Transformations, VCH Publishers (1989); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), incorporated by reference herein, are useful and recognized reference textbooks of organic synthesis known to those in the art

One of ordinary skill in the art will note that, during the reaction sequences and synthetic schemes described herein, the order of certain steps may be changed, such as the introduction and removal of protecting groups. One of ordinary skill in the art will recognise that certain groups may require protection from the reaction conditions via the use of protecting groups. Protecting groups may also be used to differentiate similar functional groups in molecules. A list of protecting groups and how to introduce and remove these groups can be found in Greene, T. W., Wuts, P. G. M., Protective Groups in Organic Synthesis, 3^{rd} edition, John Wiley & Sons: New York, 1999.

It is to be understood that, unless otherwise stated, any description of a method of treatment or prevention includes use of the compounds to provide such treatment or prevention as is described herein. It is to be further understood, unless otherwise stated, any description of a method of treatment or prevention includes use of the compounds to prepare a medicament to treat or prevent such condition. The treatment or prevention includes treatment or prevention of human or non-human animals including rodents and other disease models.

It is to be understood that, unless otherwise stated, any description of a method of treatment includes use of the compounds to provide such treatment as is described herein. It is to be further understood, unless otherwise stated, any description of a method of treatment includes use of the compounds to prepare a medicament to treat such condition. The treatment includes treatment of human or non-human animals including rodents and other disease models. As used herein, the term “subject” is interchangeable with the term “subject in need thereof”, both of which refer to a subject having a disease or having an increased risk of developing the disease. A “subject” includes a mammal. The mammal can be e.g., a human or appropriate non-human mammal, such as primate, mouse, rat, dog, cat, cow, horse, goat, camel, sheep or a pig. The subject can also be a bird or fowl. In one embodiment, the mammal is a human. A subject in need thereof can be one who has been previously diagnosed or identified as having a disease or disorder disclosed herein. A subject in need thereof can also be one who is suffering from a disease or disorder disclosed herein. Alternatively, a subject in need thereof can be one who has an increased risk of developing such disease or disorder relative to the population at large (i.e., a subject who is predisposed to developing such disorder relative to the population at large). A subject in need thereof can have a refractory or resistant a disease or disorder disclosed herein (i.e., a disease or disorder disclosed herein that does not respond or has not yet responded to treatment). The subject may be resistant at start of treatment or may become resistant during treatment. In some embodiments, the subject in need thereof received and failed all known effective therapies for a disease or disorder disclosed herein. In some embodiments, the subject in need thereof received at least one prior therapy.

As used herein, the term “treating” or “treat” describes the management and care of a patient for the purpose of combating a disease, condition, or disorder and includes the administration of a compound of the present disclosure, or a pharmaceutically acceptable salt, polymorph or solvate thereof, to alleviate the symptoms or complications of a disease, condition or disorder, or to eliminate the disease, condition or disorder. The term “treat” can also include treatment of a cell in vitro or an animal model. It is to be appreciated that references to “treating” or “treatment” include the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

It is to be understood that a compound of the present disclosure, or a pharmaceutically acceptable salt, polymorph or solvate thereof, can or may also be used to prevent a relevant disease, condition or disorder, or used to identify suitable candidates for such purposes.

As used herein, the term “preventing,” “prevent,” or “protecting against” describes reducing or eliminating the onset of the symptoms or complications of such disease, condition or disorder.

It is to be understood that one skilled in the art may refer to general reference texts for detailed descriptions of known techniques discussed herein or equivalent techniques. These texts include Ausubel et al., Current Protocols in Molecular Biology, John Wiley and Sons, Inc. (2005); Sambrook et al., Molecular Cloning, A Laboratory Manual (3^{rd} edition), Cold Spring Harbor Press, Cold Spring Harbor, New York (2000); Coligan et al., Current Protocols in Immunology, John Wiley & Sons, N.Y.; Enna et al., Current Protocols in Pharmacology John Wiley & Sons, N.Y.; Fingl et al., The Pharmacological Basis of Therapeutics (1975), Remington's Pharmaceutical Sciences, Mack Publishing Co., Easton, PA, 18^{th} edition (1990). These texts can, of course, also be referred to in making or using an aspect of the disclosure.

It is to be understood that the present disclosure also provides pharmaceutical compositions comprising any compound described herein in combination with at least one pharmaceutically acceptable excipient or carrier.

As used herein, the term “pharmaceutical composition” is a formulation containing the compounds of the present disclosure in a form suitable for administration to a subject. In one embodiment, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or salt, hydrate, solvate or isomer thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this disclosure include powders, sprays, ointments, pastes, creams, lotions, gels, solutions, patches and inhalants. In one embodiment, the active compound is mixed under sterile conditions with a pharmaceutically acceptable carrier, and with any preservatives, buffers, or propellants that are required.

As used herein, the term “pharmaceutically acceptable” refers to those compounds, anions, cations, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

It is to be understood that a pharmaceutical composition of the disclosure is formulated to be compatible with its intended route of administration. Examples of routes of administration include parenteral, e.g., intravenous, intradermal, subcutaneous, oral (e.g., ingestion), inhalation, transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

It is to be understood that a compound or pharmaceutical composition of the disclosure can be administered to a subject in many of the well-known methods currently used for chemotherapeutic treatment. For example, a compound of the disclosure may be injected into the blood stream or body cavities or taken orally or applied through the skin with patches. The dose chosen should be sufficient to constitute effective treatment but not so high as to cause unacceptable side effects. The state of the disease condition (e.g., a disease or disorder disclosed herein) and the health of the patient should preferably be closely monitored during and for a reasonable period after treatment.

As used herein, the term “therapeutically effective amount”, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

As used herein, the term “therapeutically effective amount”, refers to an amount of a pharmaceutical agent to treat or ameliorate an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

It is to be understood that, for any compound, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

The pharmaceutical compositions containing active compounds of the present disclosure may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of the active compounds into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol and sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The active compounds can be prepared with pharmaceutically acceptable carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved.

In therapeutic applications, the dosages of the pharmaceutical compositions used in accordance with the disclosure vary depending on the agent, the age, weight, and clinical condition of the recipient patient, and the experience and judgment of the clinician or practitioner administering the therapy, among other factors affecting the selected dosage. Generally, the dose should be sufficient to result in slowing, and preferably regressing, the symptoms of the disease or disorder disclosed herein and also preferably causing complete regression of the disease or disorder. Dosages can range from about 0.01 mg/kg per day to about 5000 mg/kg per day. An effective amount of a pharmaceutical agent is that which provides an objectively identifiable improvement as noted by the clinician or other qualified observer. Improvement in survival and growth indicates regression. As used herein, the term “dosage effective manner” refers to amount of an active compound to produce the desired biological effect in a subject or cell.

It is to be understood that the pharmaceutical compositions can be included in a container, pack, or dispenser together with instructions for administration.

It is to be understood that, for the compounds of the present disclosure being capable of further forming salts, all of these forms are also contemplated within the scope of the claimed disclosure.

As used herein, the term “pharmaceutically acceptable salts” refer to derivatives of the compounds of the present disclosure wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary aminonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicylic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

In some embodiments, the pharmaceutically acceptable salt is a sodium salt, a potassium salt, a calcium salt, a magnesium salt, a diethylamine salt, a choline salt, a meglumine salt, a benzathine salt, a tromethamine salt, an aminonia salt, an arginine salt, or a lysine salt.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present disclosure also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. In the salt form, it is understood that the ratio of the compound to the cation or anion of the salt can be 1:1, or any ratio other than 1:1, e.g., 3:1, 2:1, 1:2, or 1:3.

It is to be understood that all references to pharmaceutically acceptable salts include solvent addition forms (solvates) or crystal forms (polymorphs) as defined herein, of the same salt.

The compounds, or pharmaceutically acceptable salts thereof, are administered orally, nasally, transdermally, pulmonary, inhalationally, buccally, sublingually, intraperitoneally, subcutaneously, intramuscularly, intravenously, rectally, intrapleurally, intrathecally and parenterally. In one embodiment, the compound is administered orally. One skilled in the art will recognise the advantages of certain routes of administration.

The dosage regimen utilizing the compounds is selected in accordance with a variety of factors including type, species, age, weight, sex and medical condition of the patient; the severity of the condition to be treated; the route of administration, the renal and hepatic function of the patient; and the particular compound or salt thereof employed. An ordinarily skilled physician or veterinarian can readily determine and prescribe the effective amount of the drug required to prevent, counter, or arrest the progress of the condition. An ordinarily skilled physician or veterinarian can readily determine and prescribe the effective amount of the drug required to counter or arrest the progress of the condition.

Techniques for formulation and administration of the disclosed compounds of the disclosure can be found in Remington: the Science and Practice of Pharmacy, 19^{th} edition, Mack Publishing Co., Easton, PA (1995). In an embodiment, the compounds described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein.

All percentages and ratios used herein, unless otherwise indicated, are by weight. Other features and advantages of the present disclosure are apparent from the different examples. The provided examples illustrate different components and methodology useful in practicing the present disclosure. The examples do not limit the claimed disclosure. Based on the present disclosure the skilled artisan can identify and employ other components and methodology useful for practicing the present disclosure.

In the synthetic schemes described herein, compounds may be drawn with one particular configuration for simplicity. Such particular configurations are not to be construed as limiting the disclosure to one or another isomer, tautomer, regioisomer or stereoisomer, nor does it exclude mixtures of isomers, tautomers, regioisomers or stereoisomers; however, it will be understood that a given isomer, tautomer, regioisomer or stereoisomer may have a higher level of activity than another isomer, tautomer, regioisomer or stereoisomer.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

As use herein, the phrase “compound of the disclosure” refers to those compounds which are disclosed herein, both generically and specifically.

### Compounds of the Present Disclosure

In some aspects, the present disclosure provides, inter alia, a compound of Formula (I):

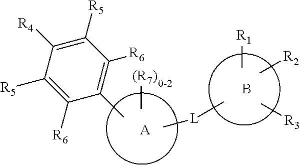

or a prodrug, solvate, or pharmaceutically acceptable salt thereof, wherein:
Ring A is 7- to 10-membered heterocyclyl or 7- to 10-membered heteroaryl;
Ring B is C₃-C₁₀ cycloalkyl, 3- to 10-membered heterocyclyl, C₆-C₁₀ aryl, or 5- to 10-membered heteroaryl;
L is absent, —C(O)—, or —CH₂—;
each R₁ and R₃ is independently H, halogen, —CN, C₁-C₆ alkyl, C₂-C₆ alkenyl, C₂-C₆ alkynyl, C₁-C₆ haloalkyl, —O—C₁-C₆ alkyl, —NH—C₁-C₆ alkyl, or C₃-C₇ cycloalkyl, wherein the alkyl, alkenyl, alkynyl, or cycloalkyl are optionally substituted with one or more R₁ₐ;
R₁ₐ is C₆-C₁₀ aryl optionally substituted with one or more halogen, —CN, or —OH;
R₂ is H, —(CH₂)ₙ—N(R₂ₐ)(R_{2b}), —(CH₂)ₙ—OH, —(CH₂)˜—O(C₁-C₆ alkyl), —(CH₂)ₙ—O(C₆-C₁₀ aryl), C₃-C₇ cycloalkyl, 3- to 10-membered heterocyclyl, C₆-C₁₀ aryl, or 5- to 10-membered heteroaryl, wherein the cycloalkyl, heterocyclyl, aryl, and heteroaryl are optionally substituted with one or more R_{2b′};
R₂ₐ is H or C₁-C₆ alkyl;
R_{2b} is C₁-C₆ alkyl, C₂-C₆ alkenyl, C₂-C₆ alkynyl, C₁-C₆ haloalkyl, or C₃-C₇ cycloalkyl, wherein the alkyl, alkenyl, alkynyl, or cycloalkyl are optionally substituted with one or more R_{2b′}, or R_{2b} is 3- to 10-membered heterocyclyl optionally substituted with one or more oxo or —(C₁-C₆ alkyl)-OH,
   or R₂ₐ and R_{2b} come together to form a 3- to 10-membered heterocyclyl optionally substituted with one or more R_{2b′};
R_{2b′} is oxo, halogen, C₁-C₆ alkyl, C₂-C₆ alkenyl, C₂-C₆ alkynyl, C₁-C₆ haloalkyl, —(CH₂)ₘ—OR_{2b′}, —(CH₂)ₘ—N(R_{2b′})(R_{2b″}), —(CH₂)ₘ—C(O)OR_{2b′}, —C(O)R_{2b′}, —C(O)N(R_{2b′})(R_{2b′}), —N(R_{2b″})C(O)R_{2b″}, C₃-C₇ cycloalkyl, 3- to 10-membered heterocyclyl, or 5- to 10-membered heteroaryl, wherein the alkyl, alkenyl, alkynyl, cycloalkyl, and heterocyclyl are optionally substituted with one or more R_{2b′};
R_{2b″} is H, oxo, —CN, —OH, C₁-C₆ alkyl, C₂-C₆ alkenyl, C₂-C₆ alkynyl, or C₁-C₆ haloalkyl, wherein the alkyl, alkenyl, and alkynyl are optionally substituted with 3- to 10-membered heterocyclyl, wherein the heterocyclyl is optionally substituted with oxo;
R_{2b′″} is H, C₁-C₆ alkyl optionally substituted with one or more —C(O)OH, —OH or —NH₂, C₂-C₆ alkenyl, C₂-C₆ alkynyl, C₃-C₇ cycloalkyl, or —C(O)C₁-C₆ alkyl;
each R₄ or R₅ is independently H, —O—C₁-C₆ alkyl, —NH—C₁-C₆ alkyl, —NH-(5- to 10-membered heteroaryl), —NH—(C₆-C₁₀ aryl), —NH—C(O)R₅ₐ, C₁-C₆ alkyl, C₂-C₆ alkenyl, C₂-C₆ alkynyl, C₁-C₆ haloalkyl, C₆-C₁₀ aryl, or 5- to 10-membered heteroaryl, wherein the alkyl, alkenyl, alkynyl, aryl, or heteroaryl are optionally substituted with one or more R₅ₐ₁,
or R₄ and R₅ come together to form a C₄-C₇ cycloalkyl or 4- to 10-membered heterocyclyl;
R₅ₐ is C₃-C₇ cycloalkyl, 3- to 10-membered heterocyclyl, C₆-C₁₀ aryl, or 5- to 10-membered heteroaryl, wherein the cycloalkyl, heterocyclyl, aryl, and heteroaryl are optionally substituted with one or more R₅ₐ₁;
R₅ₐ₁ is halogen, —CN, —(CH₂)ₚ—N(R_{5a1′})(R_{5b1′}), —O—C₁-C₆alkyl, —(CH₂)ₚ—C₃-C₇ cycloalkyl wherein the cycloalkyl is optionally substituted with one or more —C(O)OH, —OH or —NH₂, C₁-C₆ alkyl optionally substituted with one or more halogen, —C(O)OH, —OH, or —NH₂, or 3- to 10-membered heterocyclyl optionally substituted with one or more —(C₁-C₆ alkyl)-OH;
R_{5b1′} is H or C₁-C₆ alkyl;
R_{5a1′} is H, C₁-C₆ alkyl, C₂-C₆ alkenyl, C₂-C₆ alkynyl, C₁-C₆ haloalkyl, C₃-C₈ cycloalkyl, 3- to 10-membered heterocyclyl, C₆-C₁₀ aryl, or 5- to 10-membered heteroaryl, wherein the alkyl, alkenyl, alkynyl, cycloalkyl, heterocyclyl, aryl, and heteroaryl are optionally substituted with one or more R_{5a1″},
or R_{5a1′} and R_{5b1′} come together to form a 3- to 10-membered heterocyclyl optionally substituted with one or more R_{5a1″};
R_{5a1″} is oxo, —CN, —OH, C₁-C₆ alkyl optionally substituted with one or more —C(O)OH, —OH or —NH₂, C₂-C₆ alkenyl, C₂-C₆ alkynyl, C₁-C₆ haloalkyl, —C(O)OH, —C(O)C₁-C₆ alkyl, —C(O)O—C₁-C₆ alkyl, —C(O)NH₂, —C(O)NH—C₁-C₆ alkyl, —C(O)NH—C₃-C₁₀ cycloalkyl, —NH—C₁-C₆ alkyl-R_{5a1″}, —(CH₂)_{q}—(C₃-C₇ cycloalkyl), or —(CH₂)_{q}-(3- to 10-membered heterocyclyl), wherein the cycloalkyl and heterocyclyl are optionally substituted with one or more oxo, C₁-C₆ alkyl, C₂-C₆ alkenyl, C₂-C₆ alkynyl, —C(O)OH, or —OH;
R_{5a1′″} is 3- to 10-membered heterocyclyl optionally substituted with oxo;
each R₆ is independently H, halogen, —CN, C₁-C₆ alkyl, C₂-C₆ alkenyl, C₂-C₆ alkynyl, C₁-C₆ haloalkyl, —O—C₁-C₆alkyl, —NH—C₁-C₆alkyl, or C₃-C₇ cycloalkyl;
R₇ is C₁-C₆ alkyl, C₂-C₆ alkenyl, C₂-C₆ alkynyl, or C₁-C₆ haloalkyl;
m is 0, 1, 2, 3, or 4;
n is 0, 1, 2, 3, or 4;
p is 0, 1, 2, 3, or 4; and
q is 0, 1, 2, 3, or 4,
wherein at least one of R₄, R₅, or R₆ is not H; and
provided that:
(i) when Ring B is an optionally substituted 5-membered heteroaryl or 9-membered heterocyclyl and R_{5a1′} and R_{5b1′} come together to form a 5-membered heterocyclyl substituted with one R_{5a1″}, then R_{5a1″} is not —OH; or
(ii) when Ring B is an optionally substituted 9-membered heterocyclyl and R_{5a1″} and R_{5b1′} come together to form a 6-membered heterocyclyl, then R₂ is not —(CH₂)₂—OH; or

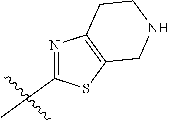 [SMILES: *c1nc2c(s1)CNCC2]

(iv) when R₅ is —NH—C(O)R₅ₐ, R₅ₐ is not a substituted 5-membered heteroaryl.

In some aspects, the present disclosure provides, inter alia, a compound of Formula (I):

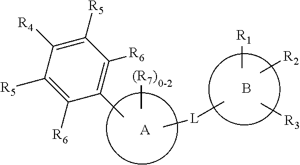

or a prodrug, solvate, or pharmaceutically acceptable salt thereof, wherein:
Ring A is 7- to 10-membered heterocyclyl or 7- to 10-membered heteroaryl;
Ring B is C₃-C₁₀ cycloalkyl, 3- to 10-membered heterocyclyl, C₆-C₁₀ aryl, or 5- to 10-membered heteroaryl;
L is absent, —C(O)—, or —CH₂—;
each R₁ and R₃ is independently H, halogen, —CN, C₁-C₆ alkyl, C₂-C₆ alkenyl, C₂-C₆ alkynyl, C₁-C₆ haloalkyl, —O—C₁-C₆ alkyl, —NH—C₁-C₆ alkyl, or C₃-C₇ cycloalkyl, wherein the alkyl, alkenyl, alkynyl, or cycloalkyl is optionally substituted with one or more R₁ₐ;
R₁ₐ is C₆-C₁₀ aryl optionally substituted with one or more halogen, —CN, or —OH;
R₂ is H, —(CH₂)ₙ—N(R₂)(R_{2b}), —(CH₂)ₙ—OH, —(CH₂)ₙ—O(C₁-C₆ alkyl), —(CH₂)ₙ—O(C₆-C₁₀ aryl), C₃-C₇ cycloalkyl, 3- to 10-membered heterocyclyl, C₆-C₁₀ aryl, or 5- to 10-membered heteroaryl, wherein the cycloalkyl, heterocyclyl, aryl, or heteroaryl is optionally substituted with one or more R_{2b′};
R₂ is H,
R_{2b} is C₁-C₆ alkyl, C₂-C₆ alkenyl, C₂-C₆ alkynyl, C₁-C₆ haloalkyl, or C₃-C₇ cycloalkyl, wherein the alkyl, alkenyl, alkynyl, or cycloalkyl is optionally substituted with one or more R_{2b′}, or R_{2b} is 3- to 10-membered heterocyclyl optionally substituted with one or more oxo or —(C₁-C₆ alkyl)-OH,
or R₂ₐ and R_{2b} come together to form a 3- to 10-membered heterocyclyl optionally substituted with one or more R_{2b′};
R_{2b′} is oxo, halogen, C₁-C₆ alkyl, C₂-C₆ alkenyl, C₂-C₆ alkynyl, C₁-C₆ haloalkyl, —(CH₂)ₘ—OR_{2b′}, —(CH₂)ₘ—N(R_{2b″})(R_{2b′″}), —(CH₂)ₘ—C(O)OR_{2b″}, —C(O)R_{2b′}, —C(O)N(R_{2b′})(R_{2b′}), —N(R_{2b″})C(O)R_{2b′}, C₃-C₇ cycloalkyl, 3- to 10-membered heterocyclyl, or 5- to 10-membered heteroaryl, wherein the alkyl, alkenyl, alkynyl, cycloalkyl, or heterocyclyl is optionally substituted with one or more R_{2b″};
R_{2b″} is H, oxo, —CN, —OH, C₁-C₆ alkyl, C₂-C₆ alkenyl, C₂-C₆ alkynyl, or C₁-C₆ haloalkyl, wherein the alkyl, alkenyl, or alkynyl is optionally substituted with 3- to 10-membered heterocyclyl, wherein the heterocyclyl is optionally substituted with oxo;
R_{2b′″} is H, C₁-C₆ alkyl optionally substituted with one or more —C(O)OH, —OH or —NH₂, C₂-C₆ alkenyl, C₂-C₆ alkynyl, C₃-C₇ cycloalkyl, or —C(O)C₁-C₆ alkyl;
each R₄ or R₅ is independently H, —O—C₁-C₆alkyl, —NH—C₁-C₆alkyl, —NH-(5- to 10-membered heteroaryl), —NH—(C₆-C₁₀ aryl), —NH—C(O)R₅ₐ, C₁-C₆ alkyl, C₂-C₆ alkenyl, C₂-C₆ alkynyl, C₁-C₆ haloalkyl, C₆-C₁₀ aryl, or 5- to 10-membered heteroaryl, wherein the alkyl, alkenyl, alkynyl, aryl, or heteroaryl is optionally substituted with one or more R₅ₐ₁,
or R₄ and R₅ come together to form a C₄-C₇ cycloalkyl or 4- to 10-membered heterocyclyl;
R₅ₐ is C₃-C₇ cycloalkyl, 3- to 10-membered heterocyclyl, C₆-C₁₀ aryl, or 5- to 10-membered heteroaryl, wherein the cycloalkyl, heterocyclyl, aryl, or heteroaryl is optionally substituted with one or more R₅ₐ₁;
R_{5a1′″} is halogen, —CN, —(CH₂)ₚ—N(R_{5a1′})(R_{5b1′}), —O—C₁-C₆ alkyl, —(CH₂)ₚ—C₃-C₇ cycloalkyl wherein the cycloalkyl is optionally substituted with one or more —C(O)OH, —OH or —NH₂, C₁-C₆ alkyl optionally substituted with one or more halogen, —C(O)OH, —OH, or —NH₂, or 3- to 10-membered heterocyclyl optionally substituted with one or more —(C₁-C₆ alkyl)-OH;
R_{5b1′} is H;
R_{5b1′} is H, C₁-C₆ alkyl, C₂-C₆ alkenyl, C₂-C₆ alkynyl, C₁-C₆ haloalkyl, C₃-C₇ cycloalkyl, 3- to 10-membered heterocyclyl, C₆-C₁₀ aryl, or 5- to 10-membered heteroaryl, wherein the alkyl, alkenyl, alkynyl, cycloalkyl, heterocyclyl, aryl, or heteroaryl is optionally substituted with one or more R_{5a1″},
or R_{5a1′} and R_{5b1′} come together to form a 3- to 10-membered heterocyclyl optionally substituted with one or more R_{5a1″};
R_{5a1″} is oxo, —CN, —OH, C₁-C₆ alkyl optionally substituted with one or more —C(O)OH, —OH or —NH₂, C₂-C₆ alkenyl, C₂-C₆ alkynyl, C₁-C₆ haloalkyl, —C(O)OH, —C(O)C₁-C₆ alkyl, —C(O)O—C₁-C₆ alkyl, —C(O)NH₂, —C(O)NH—C₁-C₆ alkyl, —C(O)NH—C₃-C₁₀ cycloalkyl, —NH—C₁-C₆ alkyl-R_{5a1′″}, —(CH₂)_{q}—(C₃-C₇ cycloalkyl), or —(CH₂)_{q}-(3- to 10-membered heterocyclyl), wherein the cycloalkyl or heterocyclyl is optionally substituted with one or more oxo, C₁-C₆ alkyl, C₂-C₆ alkenyl, C₂-C₆ alkynyl, —C(O)OH, or —OH;
R_{5a1′″} is 3- to 10-membered heterocyclyl optionally substituted with oxo;
each R₆ is independently H, halogen, —CN, C₁-C₆ alkyl, C₂-C₆ alkenyl, C₂-C₆ alkynyl, C₁-C₆ haloalkyl, —O—C₁-C₆ alkyl, —NH—C₁-C₆alkyl, or C₃-C₇ cycloalkyl;
R₇ is C₁-C₆ alkyl, C₂-C₆ alkenyl, C₂-C₆ alkynyl, or C₁-C₆ haloalkyl;
m is 0, 1, 2, 3, or 4;
n is 0, 1, 2, 3, or 4;
p is 0, 1, 2, 3, or 4; and
q is 0, 1, 2, 3, or 4,
wherein at least one of R₄, R₅, or R₆ is not H; and
provided that:
(i) when Ring B is an optionally substituted 5-membered heteroaryl or 9-membered heterocyclyl and R_{5a1′}, and R_{5b1′} come together to form a 5-membered heterocyclyl substituted with one R_{5a1″}, then R_{5a1″} is not —OH; or
(ii) when Ring B is an optionally substituted 9-membered heterocyclyl and R_{5a1′} and R_{5b1′} come together to form a 6-membered heterocyclyl, then R₂ is not —(CH₂)₂—OH; or

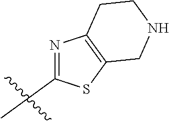 [SMILES: *c1nc2c(s1)CNCC2]

(iv) when R₅ is —NH—C(O)R₅ₐ, R₅ₐ is not a substituted 5-membered heteroaryl.

In some aspects, the present disclosure provides, inter alia, a compound of Formula (I):

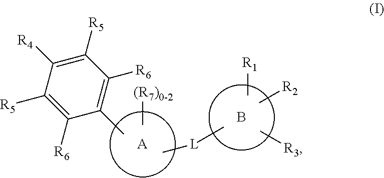

or a prodrug, solvate, or pharmaceutically acceptable salt thereof, wherein:
Ring A is 7- to 10-membered heterocyclyl or 7- to 10-membered heteroaryl;
Ring B is C₃-C₇ cycloalkyl, 3- to 10-membered heterocyclyl, C₁-C₁₀ aryl, or 5- to 10-membered heteroaryl;
L is absent, —C(O)—, or —CH₂—;
each R₁ and R₃ is independently H, halogen, —CN, C₁-C₆ alkyl, C₂-C₆ alkenyl, C₂-C₆ alkynyl, C₁-C₆ haloalkyl, —O—C₁-C₆ alkyl, —NH—C₁-C₆ alkyl, or C₃-C₇ cycloalkyl, wherein the alkyl, alkenyl, alkynyl, or cycloalkyl is optionally substituted with one or more R₁ₐ;
R₁ₐ is C₆-C₁₀ aryl optionally substituted with one or more halogen, —CN, or —OH;
R₂ is H, —(CH₂)ₙ—N(R₂ₐ)(R_{2b}), —(CH₂)ₙ—OH, —(CH₂)˜—O(C₁-C₆ alkyl), C₃-C₇ cycloalkyl, 3- to 10-membered heterocyclyl, C₆-C₁₀ aryl, or 5- to 10-membered heteroaryl, wherein the cycloalkyl, heterocyclyl, aryl, or heteroaryl is optionally substituted with one or more R_{2b′};
R₂ₐ is H;
R_{2b} is C₁-C₆ alkyl, C₂-C₆ alkenyl, C₂-C₆ alkynyl, C₁-C₆ haloalkyl, or C₃-C₇ cycloalkyl, wherein the alkyl, alkenyl, alkynyl, or cycloalkyl is optionally substituted with one or more R_{2b′}, or R_{2b} is 3- to 10-membered heterocyclyl optionally substituted with one or more oxo or —(C₁-C₆ alkyl)-OH,
or R₂ₐ and R_{2b} come together to form a 3- to 10-membered heterocyclyl optionally substituted with one or more R_{2b};
R_{2b}, is oxo, C₁-C₆ alkyl, C₂-C₆ alkenyl, C₂-C₆ alkynyl, C₁-C₆ haloalkyl, —(CH₂)ₘ—OR_{2b′}, —(CH₂)ₘ—N(R_{2b″})(R_{2b′″}), —C(O)OR_{2b′}, —C(O)R_{2b′}, —C(O)N(R_{2b′})(R_{2b′}), —N(R_{2b′})C(O)R_{2b′″}, C₃-C₇ cycloalkyl, or 3- to 10-membered heterocyclyl, wherein the alkyl, alkenyl, alkynyl, cycloalkyl, or heterocyclyl is optionally substituted with one or more R_{2b″};
R_{2b″} is H, oxo, —CN, —OH, C₁-C₆ alkyl, C₂-C₆ alkenyl, C₂-C₆ alkynyl, or C₁-C₆ haloalkyl, wherein the alkyl, alkenyl, or alkynyl is optionally substituted with 3- to 10-membered heterocyclyl, wherein the heterocyclyl is optionally substituted with oxo;
R_{2b′″} is H, C₁-C₆ alkyl, C₂-C₆ alkenyl, C₂-C₆ alkynyl, C₃-C₇ cycloalkyl, or —C(O)C₁-C₆ alkyl;
each R₄ or R₅ is independently H, —O—C₁-C₆alkyl, —NH—C₁-C₆alkyl, —NH-(5- to 10-membered heteroaryl), —NH—(C₆-C₁₀ aryl), —NH—C(O)R₅ₐ, C₁-C₆ alkyl, C₂-C₆ alkenyl, C₂-C₆ alkynyl, C₁-C₆ haloalkyl, C₆-C₁₀ aryl, or 5- to 10-membered heteroaryl, wherein the alkyl, alkenyl, alkynyl, aryl, or heteroaryl is optionally substituted with one or more R₅ₐ₁,
or R₄ and R₅ come together to form a C₄-C₇ cycloalkyl or 4- to 10-membered heterocyclyl;
R₅ₐ is C₃-C₇ cycloalkyl, 3- to 10-membered heterocyclyl, C₆-C₁₀ aryl, or 5- to 10-membered heteroaryl, wherein the cycloalkyl, heterocyclyl, aryl, or heteroaryl is optionally substituted with one or more R₅ₐ₁;
R₅ₐ₁ is halogen, —CN, —(CH₂)ₚ—N(R_{5a1′})(R_{5b1′}), —O—C₁-C₆alkyl, C₃-C₇ cycloalkyl, C₁-C₆ alkyl optionally substituted with one or more —OH or —NH₂, or 3- to 10-membered heterocyclyl optionally substituted with one or more —(C₁-C₆ alkyl)-OH;
R_{5b1′} is H;
R_{5a1′} is H, C₁-C₆ alkyl, C₂-C₆ alkenyl, C₂-C₆ alkynyl, C₁-C₆ haloalkyl, C₃-C₇ cycloalkyl, 3- to 10-membered heterocyclyl, C₆-C₁₀ aryl, or 5- to 10-membered heteroaryl, wherein the alkyl, alkenyl, alkynyl, cycloalkyl, heterocyclyl, aryl, or heteroaryl is optionally substituted with one or more R_{5a1″},
or R_{5a1′} and R_{5b1′} come together to form a 3- to 10-membered heterocyclyl optionally substituted with one or more R_{5a1″};
R_{5a1′} is oxo, —CN, —OH, C₁-C₆ alkyl, C₂-C₆ alkenyl, C₂-C₆ alkynyl, C₁-C₆ haloalkyl, —C(O)OH, —C(O)C₁-C₆ alkyl, —C(O)O—C₁-C₆ alkyl, —C(O)NH—C₁-C₆ alkyl, —C(O)NH—C₃-C₁₀ cycloalkyl, —NH—C₁-C₆ alkyl-R_{5a1′}, —(CH₂)_{q}—(C₃-C₇ cycloalkyl), or —(CH₂)_{q}-(3- to 10-membered heterocyclyl), wherein the cycloalkyl or heterocyclyl is optionally substituted with one or more oxo, C₁-C₆ alkyl, C₂-C₆ alkenyl, C₂-C₆ alkynyl, —C(O)OH, or —OH;
R_{5a1′″} is 3- to 10-membered heterocyclyl optionally substituted with oxo;
each R₆ is independently H, halogen, —CN, C₁-C₆ alkyl, C₂-C₆ alkenyl, C₂-C₆ alkynyl, C₁-C₆ haloalkyl, —O—C₁-C₆alkyl, —NH—C₁-C₆alkyl, or C₃-C₇ cycloalkyl;
R₇ is C₁-C₆ alkyl, C₂-C₆ alkenyl, C₂-C₆ alkynyl, or C₁-C₆ haloalkyl;
m is 0, 1, 2, 3, or 4;
n is 0, 1, 2, 3, or 4;
p is 0, 1, 2, 3, or 4; and
q is 0, 1, 2, 3, or 4,
   wherein at least one of R₄, R₅, or R₆ is not H; and
   provided that:
(i) when Ring B is an optionally substituted 5-membered heteroaryl or 9-membered heterocyclyl and R_{5a1′} and R_{5b1′} come together to form a 5-membered heterocyclyl substituted with one R_{5a1″}, then R_{5a1″} is not —OH; or
(ii) when Ring B is an optionally substituted 9-membered heterocyclyl and R_{5a1′} and R_{5b1′} come together to form a 6-membered heterocyclyl, then R₂ is not —(CH₂)₂—OH; or
(iii) when R₄ and R₅ come together to form a 6-membered heterocyclyl and Ring B is

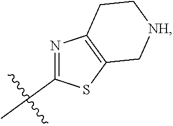 [SMILES: *c1nc2c(s1)CNCC2]

then Ring B is not substituted with —CH₃ or —CH₂CH₂—OH; or
(iv) when R₅ is —NH—C(O)R₅ₐ, R₅ₐ is not a substituted 5-membered heteroaryl.

The compound of Formula (I), wherein:
Ring A is 7- to 10-membered heterocyclyl or 7- to 10-membered heteroaryl;
Ring B is C₆-C₁₀ aryl or 5- to 10-membered heteroaryl;
L is absent;
each R₁ and R₃ is independently H or —O—C₁-C₆alkyl;
R₂ is —(CH₂)ₙ—N(R₂ₐ)(R_{2b});
R₂ₐ is H or C₁-C₆ alkyl;
R_{2b} is C₁-C₆ alkyl or C₃-C₇ cycloalkyl, wherein the alkyl or cycloalkyl is optionally substituted with one or more R_{1b′}, or R₂ₐ and R_{2b} come together to form a 3- to 10-membered heterocyclyl optionally substituted with one or more R_{2b′};
R_{2b′} is oxo, C₁-C₆ alkyl, —(CH₂)ₘ—OR_{2b′}, —(CH₂)ₙ—C(O)OR_{2b′}, —C(O)R_{2b′} or 3- to 10-membered heterocyclyl, wherein the alkyl or heterocyclyl is optionally substituted with one or more R_{2b};
R_{2b″} is H, oxo, —OH, or C₁-C₆ alkyl;
each R₄ or R₅ is independently H, —NH-(5- to 10-membered heteroaryl), C₆-C₁₀ aryl, or 5- to 10-membered heteroaryl, wherein the aryl or heteroaryl is optionally substituted with one or more R₅ₐ₁;
R_{5a1′″} is halogen, —CN, —(CH₂)ₚ—N(R_{5a1′})(R_{5b1′}), —O—C₁-C₆ alkyl, or C₁-C₆ alkyl optionally substituted with one or more halogen;
R_{5b1′} is H or C₁-C₆ alkyl;
R_{5a1′} is H, C₁-C₆ alkyl, or C₃-C₈ cycloalkyl, wherein the alkyl or cycloalkyl is optionally substituted with one or more R_{5a1″},
or R_{5a1′} and R_{5b1′} come together to form a 3- to 10-membered heterocyclyl optionally substituted with one or more R_{5a1″};
R_{5a1″} is —OH, —C(O)OH, —C(O)C₁-C₆ alkyl, or —(CH₂)_{q}-(3- to 10-membered heterocyclyl), wherein the heterocyclyl is optionally substituted with one or more oxo; and each R₆ is independently H, halogen, or C₁-C₆ alkyl;
wherein at least one of R₄, R₅, or R₆ is not H; and
provided that:
(i) when Ring B is an optionally substituted 5-membered heteroaryl or 9-membered heterocyclyl and R_{5a1″} and R_{5b1″}, come together to form a 5-membered heterocyclyl substituted with one R_{5a1″}, then R_{5a1″} is not —OH; or
(ii) when Ring B is an optionally substituted 9-membered heterocyclyl and R_{5a1′} and R_{5b1′} come together to form a 6-membered heterocyclyl, then R₂ is not —(CH₂)₂—OH; or

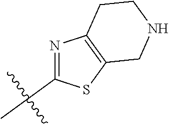 [SMILES: *c1nc2c(s1)CNCC2]

It is understood that, for a compound of Formula (I), Ring A, Ring B, L, R₁, R₂, R₃, R₄, R₅, R₆, R₇, R₁ₐ, R₂ₐ, R_{2b}, R_{2b′}, R_{2b″}, R_{2b′″}, R₅ₐ, R₅ₐ₁, R_{5a1′}, R_{5a1′″}, R_{5b1′}, m, n, p, or q can each be, where applicable, selected from the groups described herein, and any group described herein for any of Ring A, Ring B, L, R₁, R₂, R₃, R₄, R₅, R₆, R₇, R₁ₐ, R₂ₐ, R_{2b}, R_{2b′}, R_{2b″}, R_{2b′″}, R₅ₐ, R₅ₐ₁, R_{5a1′}, R_{5a1″}, R_{5a1′″}, R_{5b1′}, m, n, p, or q can be combined, where applicable, with any group described herein for one or more of the remainder of Ring A, Ring B, L, R₁, R₂, R₃, R₄, R₅, R₆, R₇, R₁ₐ, R₂ₐ, R_{2b}, R_{2b′}, R_{2b″}, R_{2b′″}, R₅ₐ, R₅ₐ₁, R_{5a1′}, R_{5a1″}, R_{5a1′″}, R_{5b1′}, m, n, p, or q.

In some embodiments, Ring A is 7- to 10-membered heterocyclyl or 7- to 10-membered heteroaryl.

In some embodiments, Ring A is 7- to 10-membered heterocyclyl. In some embodiments, Ring A is 7-membered heterocyclyl. In some embodiments, Ring A is 8-membered heterocyclyl.

In some embodiments, Ring A is 9-membered heterocyclyl. In some embodiments, Ring A is 10-membered heterocyclyl.

In some embodiments, Ring A is 7- to 10-membered heteroaryl. In some embodiments, Ring A is 7-membered heteroaryl. In some embodiments, Ring A is 8-membered heteroaryl. In some embodiments, Ring A is 9-membered heteroaryl. In some embodiments, Ring A is 10-membered heteroaryl.

In some embodiments, Ring A is indole. In some embodiments, Ring A is indoline. In some embodiments, Ring A is indazole.

In some embodiments, Ring B is C₃-C₁₀ cycloalkyl, 3- to 10-membered heterocyclyl, C₆-C₁₀ aryl, or 5- to 10-membered heteroaryl.

In some embodiments, Ring B is C₃-C₇ cycloalkyl, 3- to 10-membered heterocyclyl, C₆-C₁₀ aryl, or 5- to 10-membered heteroaryl.

In some embodiments, Ring B is C₃-C₁₀ cycloalkyl or 3- to 10-membered heterocyclyl.

In some embodiments, Ring B is C₃-C₇ cycloalkyl or 3- to 10-membered heterocyclyl.

In some embodiments, Ring B is C₃-C₁₀ cycloalkyl. In some embodiments, Ring B is C₃-C₇ cycloalkyl. In some embodiments, Ring B is C₃ cycloalkyl. In some embodiments, Ring B is C₄ cycloalkyl. In some embodiments, Ring B is C₅ cycloalkyl. In some embodiments, Ring B is C₆ cycloalkyl. In some embodiments, Ring B is C₇ cycloalkyl. In some embodiments, Ring B is C₈ cycloalkyl. In some embodiments, Ring B is C₉ cycloalkyl. In some embodiments, Ring B is C₁₀ cycloalkyl.

In some embodiments, Ring B is 3- to 10-membered heterocyclyl. In some embodiments, Ring B is 3-membered heterocyclyl. In some embodiments, Ring B is 4-membered heterocyclyl.

In some embodiments, Ring B is 5-membered heterocyclyl. In some embodiments, Ring B is 6-membered heterocyclyl. In some embodiments, Ring B is 7-membered heterocyclyl. In some embodiments, Ring B is 8-membered heterocyclyl. In some embodiments, Ring B is 9-membered heterocyclyl. In some embodiments, Ring B is 10-membered heterocyclyl.

In some embodiments, Ring B is C₆-C₁₀ aryl or 5- to 10-membered heteroaryl.

In some embodiments, Ring B is C₆-C₁₀ aryl. In some embodiments, Ring B is C₆ aryl (e.g., aryl). In some embodiments, Ring B is C₈ aryl. In some embodiments, Ring B is C₁₀ aryl.

In some embodiments, Ring B is 5- to 10-membered heteroaryl. In some embodiments, Ring B is 5-membered heteroaryl. In some embodiments, Ring B is 6-membered heteroaryl. In some embodiments, Ring B is 7-membered heteroaryl. In some embodiments, Ring B is 8-membered heteroaryl. In some embodiments, Ring B is 9-membered heteroaryl. In some embodiments, Ring B is 10-membered heteroaryl.

In some embodiments, Ring B is 3- to 10-membered heterocyclyl, C₆-C₁₀ aryl, or 5- to 10-membered heteroaryl.

In some embodiments, L is absent, —C(O)—, or —CH₂—. In some embodiments, L is absent.

In some embodiments, L is —C(O)— or —CH₂—. In some embodiments, L is —C(O)—. In some embodiments, L is —CH₂—.

In some embodiments, each R₁ and R₃ is independently H, halogen, —CN, C₁-C₆ alkyl, C₂-C₆ alkenyl, C₂-C₆ alkynyl, C₁-C₆ haloalkyl, —O—C₁-C₆alkyl, —NH—C₁-C₆alkyl, or C₃-C₇ cycloalkyl, wherein the alkyl, alkenyl, alkynyl, or cycloalkyl is optionally substituted with one or more R₁ₐ.

In some embodiments, R₁ is H, halogen, —CN, C₁-C₆ alkyl, C₂-C₆ alkenyl, C₂-C₆ alkynyl, C₁-C₆ haloalkyl, —O—C₁-C₆alkyl, —NH—C₁-C₆alkyl, or C₃-C₇ cycloalkyl, wherein the alkyl, alkenyl, alkynyl, or cycloalkyl is optionally substituted with one or more R₁ₐ.

In some embodiments, R₁ is H, halogen, —CN, C₁-C₆ alkyl, C₂-C₆ alkenyl, C₂-C₆ alkynyl, C₁-C₆ haloalkyl, —O—C₁-C₆alkyl, —NH—C₁-C₆alkyl, or C₃-C₇ cycloalkyl, wherein the alkyl, alkenyl, alkynyl, or cycloalkyl is optionally substituted with one or more R₁ₐ.

In some embodiments, R₁ is H, halogen, —CN, C₁-C₆ alkyl, C₂-C₆ alkenyl, C₂-C₆ alkynyl, C₁-C₆ haloalkyl, —O—C₁-C₆alkyl, —NH—C₁-C₆ alkyl, or C₃-C₇ cycloalkyl, wherein the alkyl, alkenyl, alkynyl, or cycloalkyl are substituted with one or more R₁ₐ.

In some embodiments, R₁ is H, halogen, —CN, C₁-C₆ alkyl, C₂-C₆ alkenyl, C₂-C₆ alkynyl, C₁-C₆ haloalkyl, —O—C₁-C₆ alkyl, —NH—C₁-C₆ alkyl, or C₃-C₇ cycloalkyl.

In some embodiments, R₁ is H.

In some embodiments, R₁ is halogen, —CN, C₁-C₆ alkyl, C₂-C₆ alkenyl, C₂-C₆ alkynyl, C₁-C₆haloalkyl, —O—C₁-C₆ alkyl, —NH—C₁-C₆ alkyl, or C₃-C₇ cycloalkyl, wherein the alkyl, alkenyl, alkynyl, or cycloalkyl is optionally substituted with one or more R₁ₐ.

In some embodiments, R₁ is halogen, —CN, C₁-C₆ alkyl, C₂-C₆ alkenyl, C₂-C₆ alkynyl, C₁-C₆ haloalkyl, —O—C₁-C₆ alkyl, —NH—C₁-C₆ alkyl, or C₃-C₇ cycloalkyl, wherein the alkyl, alkenyl, alkynyl, or cycloalkyl are substituted with one or more R₁ₐ.

In some embodiments, R₁ is halogen, —CN, C₁-C₆ alkyl, C₂-C₆ alkenyl, C₂-C₆ alkynyl, C₁-C₆ haloalkyl, —O—C₁-C₆alkyl, —NH—C₆alkyl, or C₃-C₇ cycloalkyl.

In some embodiments, R₁ is halogen or —CN.

In some embodiments, R₁ is halogen. In some embodiments, R₁ is F, Cl, Br, or I. In some embodiments, R₁ is F. In some embodiments, R₁ is C₁. In some embodiments, R₁ is Br. In some embodiments, R₁ is I.

In some embodiments, R₁ is —CN.

In some embodiments, R₁ is C₁-C₆ alkyl, C₂-C₆ alkenyl, C₂-C₆ alkynyl, C₁-C₆ haloalkyl, —O—C₁-C₆ alkyl, —NH—C₁-C₆ alkyl, or C₃-C₇ cycloalkyl, wherein the alkyl, alkenyl, alkynyl, or cycloalkyl is optionally substituted with one or more R₁ₐ.

In some embodiments, R₁ is C₁-C₆ alkyl, C₂-C₆ alkenyl, C₂-C₆ alkynyl, C₁-C₆ haloalkyl, —O—C₁-C₆ alkyl, —NH—C₁-C₆ alkyl, or C₃-C₇ cycloalkyl, wherein the alkyl, alkenyl, alkynyl, or cycloalkyl are substituted with one or more R₁ₐ.

In some embodiments, R₁ is C₁-C₆ alkyl, C₂-C₆ alkenyl, C₂-C₆ alkynyl, C₁-C₆ haloalkyl, —O—C₁-C₆ alkyl, —NH—C₁-C₆ alkyl, or C₃-C₇ cycloalkyl.

In some embodiments, R₁ is C₁-C₆ alkyl, C₂-C₆ alkenyl, C₂-C₆ alkynyl, or C₁-C₆ haloalkyl, wherein the alkyl, alkenyl, or alkynyl is optionally substituted with one or more R₁ₐ.

In some embodiments, R₁ is C₁-C₆ alkyl, C₂-C₆ alkenyl, C₂-C₆ alkynyl, or C₁-C₆ haloalkyl, wherein the alkyl, alkenyl, or alkynyl are substituted with one or more R₁ₐ.

In some embodiments, R₁ is C₁-C₆ alkyl, C₂-C₆ alkenyl, C₂-C₆ alkynyl, or C₁-C₆ haloalkyl.

In some embodiments, R₁ is C₁-C₆ alkyl optionally substituted with one or more R₁ₐ. In some embodiments, R₁ is methyl optionally substituted with one or more R₁ₐ. In some embodiments, R₁ is ethyl optionally substituted with one or more R₁ₐ. In some embodiments, R₁ is propyl optionally substituted with one or more R₁ₐ. In some embodiments, R₁ is butyl optionally substituted with one or more R₁ₐ. In some embodiments, R₁ is pentyl optionally substituted with one or more R₁ₐ. In some embodiments, R₁ is hexyl optionally substituted with one or more R₁ₐ.

In some embodiments, R₁ is isopropyl optionally substituted with one or more R₁ₐ. In some embodiments, R₁ is isobutyl optionally substituted with one or more R₁ₐ. In some embodiments, R₁ is isopentyl optionally substituted with one or more R₁ₐ. In some embodiments, R₁ is isohexyl optionally substituted with one or more R₁ₐ. In some embodiments, R₁ is secbutyl optionally substituted with one or more R₁ₐ. In some embodiments, R₁ is secpentyl optionally substituted with one or more R₁ₐ. In some embodiments, R₁ is sechexyl optionally substituted with one or more R₁ₐ. In some embodiments, R₁ is tertbutyl optionally substituted with one or more R₁ₐ.

In some embodiments, R₁ is C₁-C₆ alkyl. In some embodiments, R₁ is methyl. In some embodiments, R₁ is ethyl. In some embodiments, R₁ is propyl. In some embodiments, R₁ is butyl.

In some embodiments, R₁ is pentyl. In some embodiments, R₁ is hexyl. In some embodiments, R₁ is isopropyl. In some embodiments, R₁ is isobutyl. In some embodiments, R₁ is isopentyl. In some embodiments, R₁ is isohexyl. In some embodiments, R₁ is secbutyl. In some embodiments, R₁ is secpentyl. In some embodiments, R₁ is sechexyl. In some embodiments, R₁ is tertbutyl.

In some embodiments, R₁ is C₂-C₆ alkenyl optionally substituted with one or more R₁ₐ. In some embodiments, R₁ is C₂ alkenyl optionally substituted with one or more R₁ₐ. In some embodiments, R₁ is C₃ alkenyl optionally substituted with one or more R₁ₐ. In some embodiments, R₁ is C₄ alkenyl optionally substituted with one or more R₁ₐ. In some embodiments, R₁ is C₅ alkenyl optionally substituted with one or more R₁ₐ. In some embodiments, R₁ is C₆ alkenyl optionally substituted with one or more R₁ₐ.

In some embodiments, R₁ is C₂-C₆ alkenyl. In some embodiments, R₁ is C₂ alkenyl. In some embodiments, R₁ is C₃ alkenyl. In some embodiments, R₁ is C₄ alkenyl. In some embodiments, R₁ is C₅ alkenyl. In some embodiments, R₁ is C₆ alkenyl.

In some embodiments, R₁ is C₂-C₆ alkynyl optionally substituted with one or more R₁ₐ. In some embodiments, R₁ is C₂ alkynyl optionally substituted with one or more R₁ₐ. In some embodiments, R₁ is C₃ alkynyl optionally substituted with one or more R₁ₐ. In some embodiments, R₁ is C₄ alkynyl optionally substituted with one or more R₁ₐ. In some embodiments, R₁ is C₅ alkynyl optionally substituted with one or more R₁ₐ. In some embodiments, R₁ is C₆ alkynyl optionally substituted with one or more R₁ₐ.

In some embodiments, R₁ is C₂-C₆ alkynyl. In some embodiments, R₁ is C₂ alkynyl. In some embodiments, R₁ is C₃ alkynyl. In some embodiments, R₁ is C₄ alkynyl. In some embodiments, R₁ is C₅ alkynyl. In some embodiments, R₁ is C₆ alkynyl.

In some embodiments, R₁ is C₁-C₆ haloalkyl, wherein the alkyl is optionally substituted with one or more R₁ₐ. In some embodiments, R₁ is halomethyl, wherein the alkyl is optionally substituted with one or more R₁ₐ. In some embodiments, R₁ is haloethyl, wherein the alkyl is optionally substituted with one or more R₁ₐ. In some embodiments, R₁ is halopropyl, wherein the alkyl is optionally substituted with one or more R₁ₐ. In some embodiments, R₁ is halobutyl, wherein the alkyl is optionally substituted with one or more R₁ₐ. In some embodiments, R₁ is halopentyl, wherein the alkyl is optionally substituted with one or more R₁ₐ. In some embodiments, R₁ is halohexyl, wherein the alkyl is optionally substituted with one or more R₁ₐ.

In some embodiments, R₁ is C₁-C₆ haloalkyl. In some embodiments, R₁ is halomethyl. In some embodiments, R₁ is haloethyl. In some embodiments, R₁ is halopropyl. In some embodiments, R₁ is halobutyl. In some embodiments, R₁ is halopentyl. In some embodiments, R₁ is halohexyl.

In some embodiments, R₁ is —O—C₁-C₆ alkyl or —NH—C₁-C₆ alkyl, wherein the alkyl is optionally substituted with one or more R₁ₐ.

In some embodiments, R₁ is —O—C₁-C₆ alkyl or —NH—C₁-C₆ alkyl, wherein the alkyl is substituted with one or more R₁ₐ.

In some embodiments, R₁ is —O—C₁-C₆alkyl or —NH—C₁-C₆alkyl.

In some embodiments, R₁ is —O—C₁-C₆alkyl, wherein the alkyl is optionally substituted with one or more R₁ₐ. In some embodiments, R₁ is —O—C₁ alkyl, wherein the alkyl is optionally substituted with one or more R₁ₐ. In some embodiments, R₁ is —O—C₂ alkyl, wherein the alkyl is optionally substituted with one or more R₁ₐ. In some embodiments, R₁ is —O—C₃ alkyl, wherein the alkyl is optionally substituted with one or more R₁ₐ. In some embodiments, R₁ is —O—C₄ alkyl, wherein the alkyl is optionally substituted with one or more R₁ₐ. In some embodiments, R₁ is —O—C₅ alkyl, wherein the alkyl is optionally substituted with one or more R₁ₐ. In some embodiments, R₁ is —O—C₆alkyl, wherein the alkyl is optionally substituted with one or more R₁ₐ.

In some embodiments, R₁ is —O—C₁-C₆alkyl. In some embodiments, R₁ is —O—C₁ alkyl. In some embodiments, R₁ is —O—C₂ alkyl. In some embodiments, R₁ is —O—C₃ alkyl. In some embodiments, R₁ is —O—C₄ alkyl. In some embodiments, R₁ is —O—C₅ alkyl. In some embodiments, R₁ is —O—C₆ alkyl.

In some embodiments, R₁ is —NH—C₁-C₆alkyl, wherein the alkyl is optionally substituted with one or more R₁ₐ. In some embodiments, R₁ is —NH—C₁ alkyl, wherein the alkyl is optionally substituted with one or more R₁ₐ. In some embodiments, R₁ is —NH—C₂ alkyl, wherein the alkyl is optionally substituted with one or more R₁ₐ. In some embodiments, R₁ is —NH—C₃ alkyl, wherein the alkyl is optionally substituted with one or more R₁ₐ. In some embodiments, R₁ is —NH—C₄ alkyl, wherein the alkyl is optionally substituted with one or more R₁ₐ. In some embodiments, R₁ is —NH—C₅ alkyl, wherein the alkyl is optionally substituted with one or more R₁ₐ. In some embodiments, R₁ is —NH—C₆ alkyl, wherein the alkyl is optionally substituted with one or more R₁ₐ.

In some embodiments, R₁ is —NH—C₁-C₆alkyl. In some embodiments, R₁ is —NH—C₁ alkyl.

In some embodiments, R₁ is —NH—C₂ alkyl. In some embodiments, R₁ is —NH—C₃ alkyl. In some embodiments, R₁ is —NH—C₄ alkyl. In some embodiments, R₁ is —NH—C₅ alkyl. In some embodiments, R₁ is —NH—C₆alkyl.

In some embodiments, R₁ is C₃-C₇ cycloalkyl optionally substituted with one or more R₁ₐ.

In some embodiments, R₁ is C₃-C₇ cycloalkyl substituted with one or more R₁ₐ.

In some embodiments, R₁ is C₃-C₇ cycloalkyl.

In some embodiments, R₁ is C₇-C₇ cycloalkyl optionally substituted with one or more R₁ₐ.

In some embodiments, R₁ is C₃ cycloalkyl optionally substituted with one or more R₁ₐ. In some embodiments, R₁ is C₄ cycloalkyl optionally substituted with one or more R₁ₐ. In some embodiments, R₁ is C₅ cycloalkyl optionally substituted with one or more R₁ₐ. In some embodiments, R₁ is C, cycloalkyl optionally substituted with one or more R₁ₐ. In some embodiments, R₁ is C₇ cycloalkyl optionally substituted with one or more R₁ₐ.

In some embodiments, R₁ is C₃-C₇ cycloalkyl. In some embodiments, R₁ is C₃-C₇ cycloalkyl.

In some embodiments, R₁ is C₄ cycloalkyl. In some embodiments, R₁ is C₅ cycloalkyl. In some embodiments, R₁ is C₆ cycloalkyl. In some embodiments, R₁ is C₇ cycloalkyl.

In some embodiments, R₃ is H, halogen, —CN, C₁-C₆ alkyl, C₂-C₆ alkenyl, C₂-C₆ alkynyl, C₁-C₆ haloalkyl, —O—C₁-C₆alkyl, —NH—C₁-C₆alkyl, or C₃-C₇ cycloalkyl, wherein the alkyl, alkenyl, alkynyl, or cycloalkyl is optionally substituted with one or more R₁ₐ.

In some embodiments, R₃ is H, halogen, —CN, C₁-C₆ alkyl, C₂-C₆ alkenyl, C₂-C₆ alkynyl, C₁-C₆ haloalkyl, —O—C₁-C₆alkyl, —NH—C₁-C₆alkyl, or C₃-C₇ cycloalkyl, wherein the alkyl, alkenyl, alkynyl, or cycloalkyl are substituted with one or more R₁ₐ.

In some embodiments, R₃ is H, halogen, —CN, C₁-C₆ alkyl, C₂-C₆ alkenyl, C₂-C₆ alkynyl, C₁-C₆ haloalkyl, —O—C₁-C₆alkyl, —NH—C₁-C₆alkyl, or C₃-C₇ cycloalkyl.

In some embodiments, R₃ is H.

In some embodiments, R₃ is halogen, —CN, C₁-C₆ alkyl, C₂-C₆ alkenyl, C₂-C₆ alkynyl, C₁-C₆ haloalkyl, —O—C₁-C₆alkyl, —NH—C₁-C₆alkyl, or C₃-C₇ cycloalkyl, wherein the alkyl, alkenyl, alkynyl, or cycloalkyl is optionally substituted with one or more R₁ₐ.

In some embodiments, R₃ is halogen, —CN, C₁-C₆ alkyl, C₂-C₆ alkenyl, C₂-C₆ alkynyl, C₁-C₆ haloalkyl, —O—C₁-C₆ alkyl, —NH—C₁-C₆ alkyl, or C₃-C₇ cycloalkyl, wherein the alkyl, alkenyl, alkynyl, or cycloalkyl are substituted with one or more R₁ₐ.

In some embodiments, R₃ is halogen, —CN, C₁-C₆ alkyl, C₂-C₆ alkenyl, C₂-C₆ alkynyl, C₁-C₆ haloalkyl, —O—C₁-C₆ alkyl, —NH—C₁-C₆ alkyl, or C₃-C₇ cycloalkyl.

In some embodiments, R₃ is halogen or —CN.

In some embodiments, R₃ is halogen. In some embodiments, R₃ is F, Cl, Br, or I. In some embodiments, R₃ is F. In some embodiments, R₃ is Cl. In some embodiments, R₃ is Br. In some embodiments, R₃ is I.

In some embodiments, R₃ is —CN.

In some embodiments, R₃ is C₁-C₆ alkyl, C₂-C₆ alkenyl, C₂-C₆ alkynyl, C₁-C₆ haloalkyl, —O—C₁-C₆ alkyl, —NH—C₁-C₆ alkyl, or C₃-C₇ cycloalkyl, wherein the alkyl, alkenyl, alkynyl, or cycloalkyl is optionally substituted with one or more R₁ₐ.

In some embodiments, R₃ is C₁-C₆ alkyl, C₂-C₆ alkenyl, C₂-C₆ alkynyl, C₁-C₆ haloalkyl, —O—C₁-C₆ alkyl, —NH—C₁-C₆ alkyl, or C₃-C₇ cycloalkyl, wherein the alkyl, alkenyl, alkynyl, or cycloalkyl are substituted with one or more R₁ₐ.

In some embodiments, R₃ is C₁-C₆ alkyl, C₂-C₆ alkenyl, C₂-C₆ alkynyl, C₁-C₆ haloalkyl, —O—C₁-C₆ alkyl, —NH—C₁-C₆ alkyl, or C₃-C₇ cycloalkyl.

In some embodiments, R₃ is C₁-C₆ alkyl, C₂-C₆ alkenyl, C₂-C₆ alkynyl, or C₁-C₆ haloalkyl, wherein the alkyl, alkenyl, or alkynyl is optionally substituted with one or more R₁ₐ.

In some embodiments, R₃ is C₁-C₆ alkyl, C₂-C₆ alkenyl, C₂-C₆ alkynyl, or C₁-C₆ haloalkyl, wherein the alkyl, alkenyl, or alkynyl are substituted with one or more R₁ₐ.

In some embodiments, R₃ is C₁-C₆ alkyl, C₂-C₆ alkenyl, C₂-C₆ alkynyl, or C₁-C₆ haloalkyl.

In some embodiments, R₃ is C₁-C₆ alkyl optionally substituted with one or more R₁ₐ. In some embodiments, R₃ is methyl optionally substituted with one or more R₁ₐ. In some embodiments, R₃ is ethyl optionally substituted with one or more R₁ₐ. In some embodiments, R₃ is propyl optionally substituted with one or more R₁ₐ. In some embodiments, R₃ is butyl optionally substituted with one or more R₁ₐ. In some embodiments, R₃ is pentyl optionally substituted with one or more R₁ₐ. In some embodiments, R₃ is hexyl optionally substituted with one or more R₁ₐ.

In some embodiments, R₃ is isopropyl optionally substituted with one or more R₁ₐ. In some embodiments, R₃ is isobutyl optionally substituted with one or more R₁ₐ. In some embodiments, R₃ is isopentyl optionally substituted with one or more R₁ₐ. In some embodiments, R₃ is isohexyl optionally substituted with one or more R₁ₐ. In some embodiments, R₃ is secbutyl optionally substituted with one or more R₁ₐ. In some embodiments, R₃ is secpentyl optionally substituted with one or more R₁ₐ. In some embodiments, R₃ is sechexyl optionally substituted with one or more R₁ₐ. In some embodiments, R₃ is tertbutyl optionally substituted with one or more R₁ₐ.

In some embodiments, R₃ is C₁-C₆ alkyl. In some embodiments, R₃ is methyl. In some embodiments, R₃ is ethyl. In some embodiments, R₃ is propyl. In some embodiments, R₃ is butyl.

In some embodiments, R₃ is pentyl. In some embodiments, R₃ is hexyl. In some embodiments, R₃ is isopropyl. In some embodiments, R₃ is isobutyl. In some embodiments, R₃ is isopentyl. In some embodiments, R₃ is isohexyl. In some embodiments, R₃ is secbutyl. In some embodiments, R₃ is secpentyl. In some embodiments, R₃ is sechexyl. In some embodiments, R₃ is tertbutyl.

In some embodiments, R₃ is C₂-C₆ alkenyl optionally substituted with one or more R₁ₐ. In some embodiments, R₃ is C₂ alkenyl optionally substituted with one or more R₁ₐ. In some embodiments, R₃ is C₃ alkenyl optionally substituted with one or more R₁ₐ. In some embodiments, R₃ is C₄ alkenyl optionally substituted with one or more R₁ₐ. In some embodiments, R₃ is C₅ alkenyl optionally substituted with one or more R₁ₐ. In some embodiments, R₃ is C₆ alkenyl optionally substituted with one or more R₁ₐ.

In some embodiments, R₃ is C₂-C₆ alkenyl. In some embodiments, R₃ is C₂ alkenyl. In some embodiments, R₃ is C₃ alkenyl. In some embodiments, R₃ is C₄ alkenyl. In some embodiments, R₃ is C₅ alkenyl. In some embodiments, R₃ is C₆ alkenyl.

In some embodiments, R₃ is C₂-C₆ alkynyl optionally substituted with one or more R₁ₐ. In some embodiments, R₃ is C₂ alkynyl optionally substituted with one or more R₁ₐ. In some embodiments, R₃ is C₃ alkynyl optionally substituted with one or more R₁ₐ. In some embodiments, R₃ is C₄ alkynyl optionally substituted with one or more R₁ₐ. In some embodiments, R₃ is C₅ alkynyl optionally substituted with one or more R₁ₐ. In some embodiments, R₃ is C₆ alkynyl optionally substituted with one or more R₁ₐ.

In some embodiments, R₃ is C₂-C₆ alkynyl. In some embodiments, R₃ is C₂ alkynyl. In some embodiments, R₃ is C₃ alkynyl. In some embodiments, R₃ is C₄ alkynyl. In some embodiments, R₃ is C₅ alkynyl. In some embodiments, R₃ is C₆ alkynyl.

In some embodiments, R₃ is C₁-C₆ haloalkyl, wherein the alkyl is optionally substituted with one or more R₁ₐ. In some embodiments, R₃ is halomethyl, wherein the alkyl is optionally substituted with one or more R₁ₐ. In some embodiments, R₃ is haloethyl, wherein the alkyl is optionally substituted with one or more R₁ₐ. In some embodiments, R₃ is halopropyl, wherein the alkyl is optionally substituted with one or more R₁ₐ. In some embodiments, R₃ is halobutyl, wherein the alkyl is optionally substituted with one or more R₁ₐ. In some embodiments, R₃ is halopentyl, wherein the alkyl is optionally substituted with one or more R₁ₐ. In some embodiments, R₃ is halohexyl, wherein the alkyl is optionally substituted with one or more R₁ₐ.

In some embodiments, R₃ is C₁-C₆ haloalkyl. In some embodiments, R₃ is halomethyl. In some embodiments, R₃ is haloethyl. In some embodiments, R₃ is halopropyl. In some embodiments, R₃ is halobutyl. In some embodiments, R₃ is halopentyl. In some embodiments, R₃ is halohexyl.

In some embodiments, R₃ is —O—C₁-C₆ alkyl or —NH—C₁-C₆ alkyl, wherein the alkyl is optionally substituted with one or more R₁ₐ.

In some embodiments, R₃ is —O—C₁-C₆ alkyl or —NH—C₁-C₆ alkyl, wherein the alkyl is substituted with one or more R₁ₐ.

In some embodiments, R₃ is —O—C₁-C₆ alkyl or —NH—C₁-C₆ alkyl.

In some embodiments, R₃ is —O—C₁-C₆ alkyl, wherein the alkyl is optionally substituted with one or more R₁ₐ. In some embodiments, R₃ is —O—C₁ alkyl, wherein the alkyl is optionally substituted with one or more R₁ₐ. In some embodiments, R₃ is —O—C₂ alkyl, wherein the alkyl is optionally substituted with one or more R₁ₐ. In some embodiments, R₃ is —O—C; alkyl, wherein the alkyl is optionally substituted with one or more R₁ₐ. In some embodiments, R₃ is —O—C₄ alkyl, wherein the alkyl is optionally substituted with one or more R₁ₐ. In some embodiments, R₃ is —O—C₅ alkyl, wherein the alkyl is optionally substituted with one or more R₁ₐ. In some embodiments, R₃ is —O—C₆alkyl, wherein the alkyl is optionally substituted with one or more R₁ₐ.

In some embodiments, R₃ is —O—C₁-C₆alkyl. In some embodiments, R₃ is —O—C₁ alkyl. In some embodiments, R₃ is —O—C₂ alkyl. In some embodiments, R₃ is —O—C₃ alkyl. In some embodiments, R₃ is —O—C₄ alkyl. In some embodiments, R₃ is —O—C₅ alkyl. In some embodiments, R₃ is —O—C₆alkyl.

In some embodiments, R₃ is —NH—C₁-C₆ alkyl, wherein the alkyl is optionally substituted with one or more R₁ₐ. In some embodiments, R₃ is —NH—C₁ alkyl, wherein the alkyl is optionally substituted with one or more R₁ₐ. In some embodiments, R₃ is —NH—C₂ alkyl, wherein the alkyl is optionally substituted with one or more R₁ₐ. In some embodiments, R₃ is —NH—C₃ alkyl, wherein the alkyl is optionally substituted with one or more R₁ₐ. In some embodiments, R₃ is —NH—C₄ alkyl, wherein the alkyl is optionally substituted with one or more R₁ₐ. In some embodiments, R₃ is —NH—C₅ alkyl, wherein the alkyl is optionally substituted with one or more R₁ₐ. In some embodiments, R₃ is —NH—C₆ alkyl, wherein the alkyl is optionally substituted with one or more R₁ₐ.

In some embodiments, R₃ is —NH—C₁-C₆ alkyl. In some embodiments, R₃ is —NH—C₁ alkyl. In some embodiments, R₃ is —NH—C₂ alkyl. In some embodiments, R₃ is —NH—C₃ alkyl. In some embodiments, R₃ is —NH—C₄ alkyl. In some embodiments, R₃ is —NH—C₅ alkyl. In some embodiments, R₃ is —NH—C₆ alkyl.

In some embodiments, R₃ is C₇-C₇ cycloalkyl optionally substituted with one or more R₁ₐ.

In some embodiments, R₃ is C₃-C₂ cycloalkyl substituted with one or more R₁ₐ.

In some embodiments, R₃ is C₃-C₇ cycloalkyl.

In some embodiments, R₃ is C₃-C₇ cycloalkyl optionally substituted with one or more R₁ₐ.

In some embodiments, R₃ is C₇ cycloalkyl optionally substituted with one or more R₁ₐ. In some embodiments, R₃ is C₄ cycloalkyl optionally substituted with one or more R₁ₐ. In some embodiments, R₃ is C₅ cycloalkyl optionally substituted with one or more R₁ₐ. In some embodiments, R₃ is C₆ cycloalkyl optionally substituted with one or more R₁ₐ. In some embodiments, R₃ is C₇ cycloalkyl optionally substituted with one or more R₁ₐ.

In some embodiments, R₃ is C₃-C₇ cycloalkyl. In some embodiments, R₃ is C₃ cycloalkyl.

In some embodiments, R₃ is C₄ cycloalkyl. In some embodiments, R₃ is C₅ cycloalkyl. In some embodiments, R₃ is C₆ cycloalkyl. In some embodiments, R₃ is C₁-C₆ cycloalkyl.

In some embodiments, each R₁ and R₃ is independently H, C₁-C₆ alkyl, or —O—C₁-C₆ alkyl, wherein the alkyl is optionally substituted with one or more R₁ₐ.

In some embodiments, R₁ is H, C₁-C₆ alkyl, or —O—C₁-C₆ alkyl, wherein the alkyl is optionally substituted with one or more R₁ₐ.

In some embodiments, R₃ is H, C₁-C₆ alkyl, or —O—C₁-C₆ alkyl, wherein the alkyl is optionally substituted with one or more R₁ₐ.

In some embodiments, R₁ₐ is C₆-C₁₀ aryl optionally substituted with one or more halogen, —CN, or —OH. In some embodiments, R₁ₐ is C₆-C₁₀ aryl substituted with one or more halogen, —CN, or —OH. In some embodiments, R₁ₐ is C₆-C₁₀ aryl substituted with one halogen, —CN, or —OH. In some embodiments, R₁ₐ is C₆-C₁₀ aryl substituted with two halogen, —CN, or —OH. In some embodiments, R₁ₐ is C₆-C₁₀ aryl.

In some embodiments, R₁ₐ is C₆ aryl (e.g. phenyl) optionally substituted with one or more halogen, —CN, or —OH. In some embodiments, R₁ₐ is C₆ aryl (e.g. phenyl) substituted with one or more halogen, —CN, or —OH. In some embodiments, R₁ₐ is C₆ aryl (e.g. phenyl) substituted with one halogen, —CN, or —OH. In some embodiments, R₁ₐ is C₆ aryl (e.g. phenyl) substituted with two halogen, —CN, or —OH. In some embodiments, R₁ₐ is C₆ aryl (e.g. phenyl).

In some embodiments, R₁ₐ is C₈ aryl optionally substituted with one or more halogen, —CN, or —OH. In some embodiments, R₁ₐ is C₈ aryl substituted with one or more halogen, —CN, or —OH. In some embodiments, R₁ₐ is C₈ aryl substituted with one halogen, —CN, or —OH. In some embodiments, R₁ₐ is C₅ aryl substituted with two halogen, —CN, or —OH. In some embodiments, R₁ₐ is C₈ aryl.

In some embodiments, R₁ₐ is C₁₀ aryl optionally substituted with one or more halogen, —CN, or —OH. In some embodiments, R₁ₐ is C₁₀ aryl substituted with one or more halogen, —CN, or —OH. In some embodiments, R₁ₐ is C₁₀ aryl substituted with one halogen, —CN, or —OH. In some embodiments, R₁ₐ is C₁₀ aryl substituted with two halogen, —CN, or —OH. In some embodiments, R₁ₐ is C₁₀ aryl.

In some embodiments, R₁ₐ is C₆-C₁₀ aryl optionally substituted with one or more —CN.

In some embodiments, R₂ is H, —(CH₂)ₙ—N(R₂ₐ)(R_{2b}), —(CH₂)ₙ—OH, —(CH₂)ₙ—O(C₁-C₆ alkyl), —(CH₂)ₙ—O(C₆-C₁₀ aryl), C₃-C₇ cycloalkyl, 3- to 10-membered heterocyclyl, C₆-C₁₀ aryl, or 5- to 10-membered heteroaryl, wherein the cycloalkyl, heterocyclyl, aryl, or heteroaryl is optionally substituted with one or more R_{2b′}.

In some embodiments, R₂ is H, —(CH₂)ₙ—N(R₂ₐ)(R_{2b}), —(CH₂)ₙ—OH, —(CH₂)ₙ—O(C₁-C₆ alkyl), C₃-C₇ cycloalkyl, 3- to 10-membered heterocyclyl, C₆-C₁₀ aryl, or 5- to 10-membered heteroaryl, wherein the cycloalkyl, heterocyclyl, aryl, or heteroaryl is optionally substituted with one or more R_{2b′}.

In some embodiments, R₂ is H, —(CH₂)ₙ—N(R₂ₐ)(R_{2b}), —(CH₂)ₙ—OH, —(CH₂)ₙ—O(C₁-C₆ alkyl), —(CH₂)ₙ—O(C₆-C₁₀ aryl), C₃-C₇ cycloalkyl, 3- to 10-membered heterocyclyl, C₆-C₁₀ aryl, or 5- to 10-membered heteroaryl.

In some embodiments, R₂ is H, —(CH₂)ₙ—N(R₂ₐ)(R_{2b}), (CH₂)ₙ—OH, —(CH₂)ₙ—O(C₁-C₆ alkyl), C₃-C₇ cycloalkyl, 3- to 10-membered heterocyclyl, C₆-C₁₀ aryl, or 5- to 10-membered heteroaryl.

In some embodiments, R₂ is H.

In some embodiments, R₂ is —(CH₂)ₙ—N(R₂ₐ)(R_{2b}), —(CH₂)ₙ—OH, —(CH₂)ₙ—O(C₁-C₆ alkyl), —(CH₂)ₙ—O(C₆-C₁₀ aryl), C₃-C₇ cycloalkyl, 3- to 10-membered heterocyclyl, C₆-C₁₀ aryl, or 5- to 10-membered heteroaryl, wherein the cycloalkyl, heterocyclyl, aryl, or heteroaryl is optionally substituted with one or more R_{2b′}.

In some embodiments, R₂ is —(CH₂)ₙ—N(R₂ₐ)(R_{2b}), —(CH₂)ₙ—OH, —(CH₂)ₙ—O(C₁-C₆ alkyl), C₃-C₇ cycloalkyl, 3- to 10-membered heterocyclyl, C₆-C₁₀ aryl, or 5- to 10-membered heteroaryl, wherein the cycloalkyl, heterocyclyl, aryl, or heteroaryl is optionally substituted with one or more R_{2b′}.

In some embodiments, R₂ is —(CH₂)ₙ—N(R₂ₐ)(R_{2b}), —(CH₂)ₙ—OH, —(CH₂)ₙ—O(C₁-C₆ alkyl), —(CH₂)ₙ—O(C₆-C₁₀ aryl), C₃-C₇ cycloalkyl, 3- to 10-membered heterocyclyl, C₆-C₁₀ aryl, or 5- to 10-membered heteroaryl.

In some embodiments, R₂ is —(CH₂)ₙ—N(R₂ₐ)(R_{2b}), —(CH₂)ₙ—OH, —(CH₂)ₙ—O(C₁-C₆ alkyl), C₃-C₇ cycloalkyl, 3- to 10-membered heterocyclyl, C₆-C₁₀ aryl, or 5- to 10-membered heteroaryl.

In some embodiments, R₂ is —(CH₂)ₙ—N(R₂ₐ)(R_{2b}), —(CH₂)ₙ—OH, or —(CH₂)ₙ—O(C₁-C₆ alkyl).

In some embodiments, R₂ is —(CH₂)ₙ—N(R₂ₐ)(R_{2b}). In some embodiments, R₂ is —N(R₂ₐ)(R_{2b}). In some embodiments, R₂ is —CH₂—N(R₂ₐ)(R_{2b}). In some embodiments, R₂ is —(CH₂)₂—N(R₂ₐ)(R_{2b}). In some embodiments, R₂ is —(CH₂)₃—N(R₂ₐ)(R_{2b}). In some embodiments, R₂ is —(CH₂)₄—N(R₂ₐ)(R_{2b}).

In some embodiments, R₂ is —(CH₂)ₙ—OH. In some embodiments, R₂ is —OH. In some embodiments, R₂ is —CH₂—OH. In some embodiments, R₂ is —(CH₂)₂—OH. In some embodiments, R₂ is —(CH₂)₃—OH. In some embodiments, R₂ is —(CH₂)₄—OH.

In some embodiments, R₂ is —(CH₂)ₙ—O(C₁-C₆ alkyl). In some embodiments, R₂ is —O(C₁-C₆ alkyl). In some embodiments, R₂ is —CH₂—O(C₁-C₆ alkyl). In some embodiments, R₂ is —(CH₂)₂—O(C₁-C₆ alkyl). In some embodiments, R₂ is —(CH₂)₃—O(C₁-C₆ alkyl). In some embodiments, R₂ is —(CH₂)₄—O(C₁-C₆ alkyl).

In some embodiments, R₂ is —(CH₂)ₙ—O(C₁ alkyl). In some embodiments, R₂ is —O(C₁ alkyl). In some embodiments, R₂ is —CH₂—O(C₁ alkyl). In some embodiments, R₂ is —(CH₂)₂—O(C₁ alkyl). In some embodiments, R₂ is —(CH₂)₃—O(C₁ alkyl). In some embodiments, R₂ is —(CH₂)₄—O(C₁ alkyl).

In some embodiments, R₂ is —(CH₂)ₙ—O(C₂ alkyl). In some embodiments, R₂ is —O(C₂ alkyl). In some embodiments, R₂ is —CH₂—O(C₂ alkyl). In some embodiments, R₂ is —(CH₂)₂—O(C₂ alkyl). In some embodiments, R₂ is —(CH₂)₃—O(C₂ alkyl). In some embodiments, R₂ is —(CH₂)₄—O(C₂ alkyl).

In some embodiments, R₂ is —(CH₂)ₙ—O(C₃ alkyl). In some embodiments, R₂ is —O(C₃ alkyl). In some embodiments, R₂ is —CH₂—O(C₃ alkyl). In some embodiments, R₂ is —(CH₂)₂—O(C₃ alkyl). In some embodiments, R₂ is —(CH₂)₃—O(C₃ alkyl). In some embodiments, R₂ is —(CH₂)₄—O(C₃ alkyl).

In some embodiments, R₂ is —(CH₂)ₙ—O(C₄ alkyl). In some embodiments, R₂ is —O(C₄ alkyl). In some embodiments, R₂ is —CH₂—O(C₄ alkyl). In some embodiments, R₂ is —(CH₂)₂—O(C₄ alkyl). In some embodiments, R₂ is —(CH₂)₃—O(C₄ alkyl). In some embodiments, R₂ is —(CH₂)₄—O(C₄ alkyl).

In some embodiments, R₂ is —(CH₂)ₙ—O(C₅ alkyl). In some embodiments, R₂ is —O(C₅ alkyl). In some embodiments, R₂ is —CH₂—O(C₅ alkyl). In some embodiments, R₂ is —(CH₂)₂—O(C₅ alkyl). In some embodiments, R₂ is —(CH₂)₃—O(C₅ alkyl). In some embodiments, R₂ is —(CH₂)₄—O(C₅ alkyl).

In some embodiments, R₂ is —(CH₂)ₙ—O(C₆ alkyl). In some embodiments, R₂ is —O(C₆ alkyl). In some embodiments, R₂ is —CH₂—O(C₆ alkyl). In some embodiments, R₂ is —(CH₂)₂—O(C₆ alkyl). In some embodiments, R₂ is —(CH₂)₃—O(C₆ alkyl). In some embodiments, R₂ is —(CH₂)₄—O(C₆ alkyl).

In some embodiments, R₂ is —(CH₂)ₙ—O(C₆-C₁₀ aryl), wherein the aryl is optionally substituted with one or more R_{2b′}.

In some embodiments, R₂ is —(CH₂)ₙ—O(C₆ aryl), wherein the aryl is optionally substituted with one or more R_{2b′}. In some embodiments, R₂ is —(CH₂)ₙ—O(C₅ aryl), wherein the aryl is optionally substituted with one or more R_{2b′}. In some embodiments, R₂ is —(CH₂)ₙ—O(C₁₀ aryl), wherein the aryl is optionally substituted with one or more R_{2b′}.

In some embodiments, R₂ is —(CH₂)ₙ—O(C₆-C₁₀ aryl).

In some embodiments, R₂ is —(CH₂)ₙ—O(C₆ aryl). In some embodiments, R₂ is —(CH₂)ₙ—O(C₈ aryl). In some embodiments, R₂ is —(CH₂)ₙ—O(C₁₀ aryl).

In some embodiments, R₂ is C₃-C₇ cycloalkyl, 3- to 10-membered heterocyclyl, C₆-C₁₀ aryl, or 5- to 10-membered heteroaryl, wherein the cycloalkyl, heterocyclyl, aryl, or heteroaryl is optionally substituted with one or more R_{2b′}.

In some embodiments, R₂ is C₃-C₇ cycloalkyl, 3- to 10-membered heterocyclyl, C₆-C₁₀ aryl, or 5- to 10-membered heteroaryl.

In some embodiments, R₂ is C₃-C₇ cycloalkyl or 3- to 10-membered heterocyclyl, wherein the cycloalkyl or heterocyclyl is optionally substituted with one or more R_{2b′}.

In some embodiments, R₂ is C₃-C₇ cycloalkyl or 3- to 10-membered heterocyclyl. In some embodiments, R₂ is C₃-C₇ cycloalkyl optionally substituted with one or more R_{2b′}. In some embodiments, R₂ is C₃ cycloalkyl optionally substituted with one or more R_{2b′}. In some embodiments, R₂ is C₄ cycloalkyl optionally substituted with one or more R_{2b′}. In some embodiments, R₂ is C₅ cycloalkyl optionally substituted with one or more R_{2b′}. In some embodiments, R₂ is C₆ cycloalkyl optionally substituted with one or more R_{2b′}. In some embodiments, R₂ is C₇ cycloalkyl optionally substituted with one or more R_{2b′}.

In some embodiments, R₂ is C₃-C₇ cycloalkyl. In some embodiments, R₂ is C₃ cycloalkyl. In some embodiments, R₂ is C₄ cycloalkyl. In some embodiments, R₂ is C₅ cycloalkyl. In some embodiments, R₂ is C₆ cycloalkyl. In some embodiments, R₂ is C₃ cycloalkyl.

In some embodiments, R₂ is 3- to 10-membered heterocyclyl optionally substituted with one or more R_{2b′}. In some embodiments, R₂ is 3-membered heterocyclyl optionally substituted with one or more R_{2b′}. In some embodiments, R₂ is 4-membered heterocyclyl optionally substituted with one or more R_{2b′}. In some embodiments, R₂ is 5-membered heterocyclyl optionally substituted with one or more R_{2b′}. In some embodiments, R₂ is 6-membered heterocyclyl optionally substituted with one or more R_{2b′}. In some embodiments, R₂ is 7-membered heterocyclyl optionally substituted with one or more R_{2b′}. In some embodiments, R₂ is 8-membered heterocyclyl optionally substituted with one or more R_{2b′}. In some embodiments, R₂ is 9-membered heterocyclyl optionally substituted with one or more R_{2b′}. In some embodiments, R₂ is 10-membered heterocyclyl optionally substituted with one or more R_{2b′}.

In some embodiments, R₂ is 3- to 10-membered heterocyclyl. In some embodiments, R₂ is 3-membered heterocyclyl. In some embodiments, R₂ is 4-membered heterocyclyl. In some embodiments, R₂ is 5-membered heterocyclyl. In some embodiments, R₂ is 6-membered heterocyclyl. In some embodiments, R₂ is 7-membered heterocyclyl. In some embodiments, R₂ is 8-membered heterocyclyl. In some embodiments, R₂ is 9-membered heterocyclyl. In some embodiments, R₂ is 10-membered heterocyclyl.

In some embodiments, R₂ is C₆-C₁₀ aryl or 5- to 10-membered heteroaryl, wherein the aryl or heteroaryl is optionally substituted with one or more R_{2b′}.

In some embodiments, R₂ is C₆-C₁₀ aryl or 5- to 10-membered heteroaryl.

In some embodiments, R₂ is C₆-C₁₀ aryl optionally substituted with one or more R_{2b′}.

In some embodiments, R₂ is C₆ aryl (e.g., phenyl) optionally substituted with one or more R_{2b′}. In some embodiments, R₂ is C₈ aryl optionally substituted with one or more R_{2b′}. In some embodiments, R₂ is C₁₀ aryl optionally substituted with one or more R_{2b′.}

In some embodiments, R₂ is C₆-C₁₀ aryl. In some embodiments, R₂ is C₆ aryl (e.g., phenyl).

In some embodiments, R₂ is C₅ aryl. In some embodiments, R₂ is C₁₀ aryl.

In some embodiments, R₂ is 5- to 10-membered heteroaryl optionally substituted with one or more R_{2b}. In some embodiments, R₂ is 5-membered heteroaryl optionally substituted with one or more R₂₆. In some embodiments, R₂ is 6-membered heteroaryl optionally substituted with one or more R_{2b′}. In some embodiments, R₂ is 7-membered heteroaryl optionally substituted with one or more R_{2b}. In some embodiments, R₂ is 8-membered heteroaryl optionally substituted with one or more R_{2b′}. In some embodiments, R₂ is 9-membered heteroaryl optionally substituted with one or more R_{2b′}. In some embodiments, R₂ is 10-membered heteroaryl optionally substituted with one or more R_{2b′}.

In some embodiments, R₂ is 5- to 10-membered heteroaryl. In some embodiments, R₂ is 5-membered heteroaryl. In some embodiments, R₂ is 6-membered heteroaryl. In some embodiments, R₂ is 7-membered heteroaryl. In some embodiments, R₂ is 8-membered heteroaryl. In some embodiments, R₂ is 9-membered heteroaryl. In some embodiments, R₂ is 10-membered heteroaryl.

In some embodiments, R₂ is H, —(CH₂)ₙ—N(R₂ₐ)(R_{2b}), C₃-C₇ cycloalkyl, 3- to 10-membered heterocyclyl, C₆-C₁₀ aryl, or 5- to 10-membered heteroaryl, wherein the cycloalkyl, heterocyclyl, aryl, or heteroaryl is optionally substituted with one or more R_{2b′}.

In some embodiments, R₂ₐ is H or C₁-C₆ alkyl.

In some embodiments, R₂ₐ is H.

In some embodiments, R₂ₐ is C₁-C₆ alkyl. In some embodiments, R₂ₐ is methyl. In some embodiments, R₂ₐ is ethyl. In some embodiments, R₂ₐ is propyl. In some embodiments, R₂ₐ is butyl. In some embodiments, R₂ₐ is pentyl. In some embodiments, R₂ₐ is hexyl. In some embodiments, R_{2b} is isopropyl. In some embodiments, R_{2b} is isobutyl. In some embodiments, R₂ₐ is isopentyl. In some embodiments, R₂ₐ is isohexyl. In some embodiments, R₂ is secbutyl. In some embodiments, R₂ₐ is secpentyl. In some embodiments, R₂ₐ is sechexyl. In some embodiments, R₂ₐ is tertbutyl.

In some embodiments, R_{2b} is C₁-C₆ alkyl, C₂-C₆ alkenyl, C₂-C₆ alkynyl, C₁-C₆ haloalkyl, or C₃-C₇ cycloalkyl, wherein the alkyl, alkenyl, alkynyl, or cycloalkyl is optionally substituted with one or more R_{2b′}, or R_{2b} is 3- to 10-membered heterocyclyl optionally substituted with one or more oxo or —(C₁-C₆ alkyl)-OH.

In some embodiments, R_{2b} is C₁-C₆ alkyl, C₂-C₆ alkenyl, C₂-C₆ alkynyl, C₁-C₆ haloalkyl, or C₃-C₇ cycloalkyl, wherein the alkyl, alkenyl, alkynyl, or cycloalkyl is optionally substituted with one or more R_{2b′}.

In some embodiments, R_{2b} is C₁-C₆ alkyl, C₂-C₆ alkenyl, C₂-C₆ alkynyl, C₁-C₆ haloalkyl, or C₃-C₇ cycloalkyl.

In some embodiments, R_{2b} is C₁-C₆ alkyl, C₂-C₆ alkenyl, C₂-C₆ alkynyl, or C₁-C₆ haloalkyl, wherein the alkyl, alkenyl, or alkynyl is optionally substituted with one or more R_{2b′}.

In some embodiments, R_{2b} is C₁-C₆ alkyl, C₂-C₆ alkenyl, C₂-C₆ alkynyl, or C₁-C₆ haloalkyl.

In some embodiments, R_{2b} is C₁-C₆ alkyl, C₂-C₆ alkenyl, or C₂-C₆ alkynyl, wherein the alkyl, alkenyl, or alkynyl is optionally substituted with one or more R_{2b′}.

In some embodiments, R_{2b} is C₁-C₆ alkyl, C₂-C₆ alkenyl, or C₂-C₆ alkynyl.

In some embodiments, R₂ₐ is C₁-C₆ alkyl optionally substituted with one or more R_{2b′}. In some embodiments, R_{2b} is methyl optionally substituted with one or more R_{2b′}. In some embodiments, R_{2b} is ethyl optionally substituted with one or more R_{2b′}. In some embodiments, R_{2b} is propyl optionally substituted with one or more R_{2b′}. In some embodiments, R_{2b} is butyl optionally substituted with one or more R_{2b′}. In some embodiments, R_{2b} is pentyl optionally substituted with one or more R_{2b′}. In some embodiments, R_{2b} is hexyl optionally substituted with one or more R_{2b′}. In some embodiments, R_{2b} is isopropyl optionally substituted with one or more R_{2b′}. In some embodiments, R_{2b} is isobutyl optionally substituted with one or more R_{2b′}. In some embodiments, R_{2b} is isopentyl optionally substituted with one or more R_{2b′}. In some embodiments, R_{2b} is isohexyl optionally substituted with one or more R_{2b′}. In some embodiments, R_{2b} is secbutyl optionally substituted with one or more R_{2b′}. In some embodiments, R_{2b} is secpentyl optionally substituted with one or more R_{2b′}. In some embodiments, R_{2b} is sechexyl optionally substituted with one or more R_{2b′}. In some embodiments, R_{2b} is tertbutyl optionally substituted with one or more R_{2b′}.

In some embodiments, R_{2b} is C₁-C₆ alkyl. In some embodiments, R_{2b} is methyl. In some embodiments, R₂ₐ is ethyl. In some embodiments, R₂ₐ is propyl. In some embodiments, R_{2b} is butyl. In some embodiments, R_{2b} is pentyl. In some embodiments, R_{2b} is hexyl. In some embodiments, R_{2b} is isopropyl. In some embodiments, R_{2b} is isobutyl. In some embodiments, R_{2b} is isopentyl. In some embodiments, R_{2b} is isohexyl. In some embodiments, R_{2b} is secbutyl. In some embodiments, R₂ is secpentyl. In some embodiments, R_{2b} is sechexyl. In some embodiments, R_{2b} is tertbutyl.

In some embodiments, R_{2b} is C₂-C₆ alkenyl optionally substituted with one or more R_{2b′}.

In some embodiments, R_{2b} is C₂ alkenyl optionally substituted with one or more R_{2b′}. In some embodiments, R_{2b} is C₃ alkenyl optionally substituted with one or more R_{2b′}. In some embodiments, R_{2b} is C₄ alkenyl optionally substituted with one or more R_{2b′}. In some embodiments, R_{2b} is C₅ alkenyl optionally substituted with one or more R_{2b′}. In some embodiments, R_{2b} is C₆ alkenyl optionally substituted with one or more R_{2b′}.

In some embodiments, R_{2b} is C₂-C₆ alkenyl. In some embodiments, R_{2b} is C₂ alkenyl. In some embodiments, R_{2b} is C₃ alkenyl. In some embodiments, R_{2b} is C₄ alkenyl. In some embodiments, R_{2b} is C₅ alkenyl. In some embodiments, R_{2b} is C₆ alkenyl.

In some embodiments, R_{2b} is C₂-C₆ alkynyl optionally substituted with one or more R_{2b′}. In some embodiments, R_{2b} is C₂ alkynyl optionally substituted with one or more R_{2b′}. In some embodiments, R_{2b} is C₃ alkynyl optionally substituted with one or more R_{2b′}. In some embodiments, R_{2b} is C₄ alkynyl optionally substituted with one or more R_{2b′}. In some embodiments, R_{2b} is C₅ alkynyl optionally substituted with one or more R_{2b′}. In some embodiments, R_{2b} is C₆ alkynyl optionally substituted with one or more R_{2b}.

In some embodiments, R_{2b} is C₂-C₆ alkynyl. In some embodiments, R_{2b} is C₂ alkynyl. In some embodiments, R_{2b} is C₃ alkynyl. In some embodiments, R_{2b} is C₄ alkynyl. In some embodiments, R_{2b} is C₅ alkynyl. In some embodiments, R_{2b} is C, alkynyl.

In some embodiments, R_{2b} is C₁-C₆ haloalkyl optionally substituted with one or more R_{2b′}.

In some embodiments, R_{2b} is halomethyl optionally substituted with one or more R_{2b′}. In some embodiments, R_{2b} is haloethyl optionally substituted with one or more R_{2b′}. In some embodiments, R_{2b} is halopropyl optionally substituted with one or more R_{2b′}. In some embodiments, R_{2b} is halobutyl optionally substituted with one or more R_{2b′}. In some embodiments, R_{2b} is halopentyl optionally substituted with one or more R_{2b′}. In some embodiments, R_{2b} is halohexyl optionally substituted with one or more R_{2b′}.

In some embodiments, R_{2b} is C₁-C₆ haloalkyl. In some embodiments, R_{2b} is halomethyl. In some embodiments, R_{2b} is haloethyl. In some embodiments, R_{2b} is halopropyl. In some embodiments, R_{2b} is halobutyl. In some embodiments, R_{2b} is halopentyl. In some embodiments, R_{2b} is halohexyl.

In some embodiments, R_{2b} is C₃-C₇ cycloalkyl optionally substituted with one or more R_{2b′}. In some embodiments, R_{2b} is C₃ cycloalkyl optionally substituted with one or more R_{2b′}. In some embodiments, R_{2b} is C₄ cycloalkyl optionally substituted with one or more R_{2b′}. In some embodiments, R_{2b} is C₅ cycloalkyl optionally substituted with one or more R_{2b′}. In some embodiments, R_{2b} is C₆ cycloalkyl optionally substituted with one or more R_{2b′}. In some embodiments, R_{2b} is C₇ cycloalkyl optionally substituted with one or more R_{2b}.

In some embodiments, R_{2b} is C₃-C₇ cycloalkyl. In some embodiments, R_{2b} is C₃ cycloalkyl.

In some embodiments, R_{2b} is C₄ cycloalkyl. In some embodiments, R_{2b} is C₅ cycloalkyl. In some embodiments, R_{2b} is C₆ cycloalkyl. In some embodiments, R_{2b} is C₇ cycloalkyl.

In some embodiments, R_{2b} is 3- to 10-membered heterocyclyl optionally substituted with one or more oxo or —(C₁-C₆ alkyl)-OH. In some embodiments, R_{2b} is 3- to 10-membered heterocyclyl substituted with one or more oxo or —(C₁-C₆ alkyl)-OH. In some embodiments, R_{2b} is 3- to 10-membered heterocyclyl.

In some embodiments, R_{2b} is 3-membered heterocyclyl optionally substituted with one or more oxo or —(C₁-C₆ alkyl)-OH. In some embodiments, R_{2b} is 3-membered heterocyclyl substituted with one or more oxo or —(C₁-C₆ alkyl)-OH. In some embodiments, R is 3-membered heterocyclyl.

In some embodiments, R_{2b} is 4-membered heterocyclyl optionally substituted with one or more oxo or —(C₁-C₆ alkyl)-OH. In some embodiments, R_{2b} is 4-membered heterocyclyl substituted with one or more oxo or —(C₁-C₆ alkyl)-OH. In some embodiments, R_{2b} is 4-membered heterocyclyl.

In some embodiments, R_{2b} is 5-membered heterocyclyl optionally substituted with one or more oxo or —(C₁-C₆ alkyl)-OH. In some embodiments, R_{2b} is 5-membered heterocyclyl substituted with one or more oxo or —(C₁-C₆ alkyl)-OH. In some embodiments, R_{2b} is 5-membered heterocyclyl.

In some embodiments, R_{2b} is 6-membered heterocyclyl optionally substituted with one or more oxo or —(C₁-C₆ alkyl)-OH. In some embodiments, R_{2b} is 6-membered heterocyclyl substituted with one or more oxo or —(C₁-C₆ alkyl)-OH. In some embodiments, R_{2b} is 6-membered heterocyclyl.

In some embodiments, R_{2b} is 7-membered heterocyclyl optionally substituted with one or more oxo or —(C₁-C₆ alkyl)-OH. In some embodiments, R_{2b} is 7-membered heterocyclyl substituted with one or more oxo or —(C₁-C₆ alkyl)-OH. In some embodiments, R_{2b} is 7-membered heterocyclyl.

In some embodiments, R_{2b} is 8-membered heterocyclyl optionally substituted with one or more oxo or —(C₁-C₆ alkyl)-OH. In some embodiments, R_{2b} is 8-membered heterocyclyl substituted with one or more oxo or —(C₁-C₆ alkyl)-OH. In some embodiments, R_{2b} is 8-membered heterocyclyl.

In some embodiments, R_{2b} is 9-membered heterocyclyl optionally substituted with one or more oxo or —(C₁-C₆ alkyl)-OH. In some embodiments, R_{2b} is 9-membered heterocyclyl substituted with one or more oxo or —(C₁-C₆ alkyl)-OH. In some embodiments, R_{2b} is 9-membered heterocyclyl.

In some embodiments, R_{2b} is 10-membered heterocyclyl optionally substituted with one or more oxo or —(C₁-C₆ alkyl)-OH. In some embodiments, R_{2b} is 10-membered heterocyclyl substituted with one or more oxo or —(C₁-C₆ alkyl)-OH. In some embodiments, R_{2b} is 10-membered heterocyclyl.

In some embodiments, R_{2b} is C₁-C₆ alkyl or C₃-C₇ cycloalkyl, wherein the alkyl or cycloalkyl is optionally substituted with one or more R_{2b′}.

In some embodiments, R_{2b} is 3- to 10-membered heterocyclyl substituted with one or more oxo or —(C₁-C₆ alkyl)-OH.

In some embodiments, R₂ₐ and R_{2b} come together to form a 3- to 10-membered heterocyclyl optionally substituted with one or more R_{2b′}. In some embodiments, R₂ and R_{2b} come together to form a 3- to 10-membered heterocyclyl substituted with one or more R_{2b′}. In some embodiments, R₂ₐ and R_{2b} come together to form a 3- to 10-membered heterocyclyl.

In some embodiments, R₂ₐ and R_{2b} come together to form a 3-membered heterocyclyl optionally substituted with one or more R_{2b′}. In some embodiments, R₂ₐ and R_{2b} come together to form a 3-membered heterocyclyl substituted with one or more R_{2b′}. In some embodiments, R₂ₐ and R_{2b} come together to form a 3-membered heterocyclyl.

In some embodiments, R₂ₐ and R_{2b} come together to form a 4-membered heterocyclyl optionally substituted with one or more R_{2b′}. In some embodiments, R₂ₐ and R_{2b} come together to form a 4-membered heterocyclyl substituted with one or more R_{2b′}. In some embodiments, R₂ₐ and R_{2b} come together to form a 4-membered heterocyclyl.

In some embodiments, R₂ₐ and R_{2b} come together to form a 5-membered heterocyclyl optionally substituted with one or more R_{2b′}. In some embodiments, R₂ₐ and R_{2b} come together to form a 5-membered heterocyclyl substituted with one or more R_{2b′}. In some embodiments, R₂ₐ and R_{2b} come together to form a 5-membered heterocyclyl.

In some embodiments, R₂ₐ and R_{2b} come together to form a 6-membered heterocyclyl optionally substituted with one or more R_{2b}. In some embodiments, R₂ₐ and R_{2b} come together to form a 6-membered heterocyclyl substituted with one or more R_{2b′}. In some embodiments, R₂ₐ and R_{2b} come together to form a 6-membered heterocyclyl.

In some embodiments, R₂ₐ and R_{2b} come together to form a 7-membered heterocyclyl optionally substituted with one or more R_{2b′}. In some embodiments, R₂ₐ and R_{2b} come together to form a 7-membered heterocyclyl substituted with one or more R_{2b′}. In some embodiments, R₂ₐ and R_{2b} come together to form a 7-membered heterocyclyl.

In some embodiments, R₂ₐ and R_{2b} come together to form a 8-membered heterocyclyl optionally substituted with one or more R_{2b′}. In some embodiments, R₂ₐ and R_{2b} come together to form a 8-membered heterocyclyl substituted with one or more R_{2b′}. In some embodiments, R₂, and R_{2b} come together to form a 8-membered heterocyclyl.

In some embodiments, R₂ₐ and R_{2b} come together to form a 9-membered heterocyclyl optionally substituted with one or more R_{2b′}. In some embodiments, R₂ₐ and R_{2b} come together to form a 9-membered heterocyclyl substituted with one or more R_{2b′}. In some embodiments, R₂ₐ and R_{2b} come together to form a 9-membered heterocyclyl.

In some embodiments, R₂ₐ and R_{2b} come together to form a 10-membered heterocyclyl optionally substituted with one or more R_{2b′}. In some embodiments, R₂ₐ and R_{2b} come together to form a 10-membered heterocyclyl substituted with one or more R_{2b′}. In some embodiments, R₂ₐ and R_{2b} come together to form a 10-membered heterocyclyl.

In some embodiments, R_{2b′} is oxo, halogen, C₁-C₆ alkyl, C₂-C₆ alkenyl, C₂-C₆ alkynyl, C₁-C₆ haloalkyl, —(CH₂)ₘ—OR_{2b′}, —(CH₂)ₘ—N(R_{2b′})(R_{2b″}), —(CH₂)ₘ—C(O)OR_{2b′″}, —C(O)R_{2b′}, —C(O)N(R_{2b′})(R_{2b′}), —N(R_{2b′})C(O)R_{2b′″}, C₃-C₇ cycloalkyl, 3- to 10-membered heterocyclyl, or 5- to 10-membered heteroaryl, wherein the alkyl, alkenyl, alkynyl, cycloalkyl, or heterocyclyl is optionally substituted with one or more R_{2b}.

In some embodiments, R_{2b′} is oxo, C₁-C₆ alkyl, C₂-C₆ alkenyl, C₂-C₆ alkynyl, C₁-C₆ haloalkyl, —(CH₂)ₘ—OR_{2b′}, —(CH₂)ₘ—N(R_{2b′})(R_{2b′}), —C(O)OR_{2b′}, —C(O)R_{2b′}, —C(O)N(R_{2b″})(R_{2b′}), —N(R_{2b′})C(O)R_{2b′}, C₃-C₇ cycloalkyl, or 3- to 10-membered heterocyclyl, wherein the alkyl, alkenyl, alkynyl, cycloalkyl, or heterocyclyl is optionally substituted with one or more R_{2b′}.

In some embodiments, R_{2b″} is oxo, halogen, C₁-C₆ alkyl, C₂-C₆ alkenyl, C₂-C₆ alkynyl, C₁-C₆ haloalkyl, —(CH₂), —OR_{2b′}, —(CH₂)ₘ—N(R_{2b′})(R_{2b″}), —(CH₂)ₘ—C(O)OR_{2b′}, —C(O)R_{2b′}, —C(O)N(R_{2b′})(R_{2b′}), —N(R_{2b′})C(O)R_{2b′″}, C₃-C₇ cycloalkyl, 3- to 10-membered heterocyclyl, or 5- to 10-membered heteroaryl.

In some embodiments, R_{2b′} is oxo, C₁-C₆ alkyl, C₂-C₆ alkenyl, C₂-C₆ alkynyl, C₁-C₆ haloalkyl, —(CH₂)ₘ—OR_{2b′}, —(CH₂)ₙ—N(R_{2b′})(R_{2b′″}), —C(O)OR_{2b′}, —C(O)R_{2b″}, —C(O)N(R_{2b′})(R_{2b′}), —N(R_{2b′})C(O)R_{2b′}, C₃-C₇ cycloalkyl, or 3- to 10-membered heterocyclyl.

In some embodiments, R_{2b′} is oxo.

In some embodiments, R_{2b′} is halogen. In some embodiments, R_{2b′} is F, Cl, Br, or I. In some embodiments, R_{2b′} is F. In some embodiments, R_{2b′} is C₁. In some embodiments, R_{2b′} is Br.

In some embodiments, R_{2b′} is I.

In some embodiments, R_{2b′} is C₁-C₆ alkyl, C₂-C₆ alkenyl, C₂-C₆ alkynyl, C₁-C₆ haloalkyl, —(CH₂)ₘ—OR_{2b′}, —(CH₂)ₘ—N(R_{2b′})(R_{2b′}), —(CH₂)ₘ—C(O)OR_{2b′}, —C(O)R_{2b′}, —C(O)N(R_{2b′})(R_{2b″}), —N(R_{2b′})C(O)R_{2b″}, C₃-C₇ cycloalkyl, 3- to 10-membered heterocyclyl, or 5- to 10-membered heteroaryl, wherein the alkyl, alkenyl, alkynyl, cycloalkyl, or heterocyclyl is optionally substituted with one or more R_{2b″}.

In some embodiments, R_{2b′} is C₁-C₆ alkyl, C₂-C₆ alkenyl, C₂-C₆ alkynyl, C₁-C₆ haloalkyl, —(CH₂)ₘ—OR_{2b″}, —(CH₂)ₘ—N(R_{2b′})(R_{2b″}), —C(O)OR_{2b′}, —C(O)R_{2b″}, —C(O)N(R_{2b′})(R_{2b″}), —N(R_{2b′})C(O)R_{2b′}, C₃-C₇ cycloalkyl, or 3- to 10-membered heterocyclyl, wherein the alkyl, alkenyl, alkynyl, cycloalkyl, or heterocyclyl is optionally substituted with one or more R_{2b′}.

In some embodiments, R_{2b′} is C₁-C₆ alkyl, C₂-C₆ alkenyl, C₂-C₆ alkynyl, C₁-C₆ haloalkyl, —(CH₂)ₘ—OR_{2b′}, —(CH₂)ₘ—N(R_{2b′})(R_{2b′}), —(CH₂)ₘ—C(O)OR_{2b′}, —C(O)R_{2b′}, —C(O)N(R_{2b′})(R_{2b″}), —N(R_{2b′})C(O)R_{2b″}, C₃-C₇ cycloalkyl, 3- to 10-membered heterocyclyl, or 5- to 10-membered heteroaryl.

In some embodiments, R_{2b′} is C₁-C₆ alkyl, C₂-C₆ alkenyl, C₂-C₆ alkynyl, C₁-C₆ haloalkyl, —(CH₂)ₘ—OR_{2b′}, —(CH₂)ₘ—N(R_{2b′})(R_{2b′″}), —C(O)OR_{2b′}, —C(O)R_{2b′}, —C(O)N(R_{2b′})(R_{2b′″}), —N(R_{2b′})C(O)R_{2b′}, C₃-C₇ cycloalkyl, or 3- to 10-membered heterocyclyl.

In some embodiments, R_{2b′} is C₁-C₆ alkyl, C₂—C, alkenyl, C₂-C₆ alkynyl, or C₁-C₆ haloalkyl, wherein the alkyl, alkenyl, or alkynyl is optionally substituted with one or more R_{2b″}.

In some embodiments, R_{2b′} is C₁-C₆ alkyl, C₂-C₆ alkenyl, C₂-C₆ alkynyl, or C₂-C₆ haloalkyl.

In some embodiments, R_{2b′} is C₁-C₆ alkyl, C₂-C₆ alkenyl, or C₂-C₆ alkynyl, wherein the alkyl, alkenyl, or alkynyl is optionally substituted with one or more R_{2b′}.

In some embodiments, R_{2b′} is C₁-C₆ alkyl, C₂-C₆ alkenyl, or C₂-C₆ alkynyl.

In some embodiments, R_{2b′} is C₁-C₆ alkyl optionally substituted with one or more R_{2b′}. In some embodiments, R_{2b′} is methyl optionally substituted with one or more R_{2b′}. In some embodiments, R_{2b″} is ethyl optionally substituted with one or more R_{2b′}. In some embodiments, R_{2b′} is propyl optionally substituted with one or more R_{2b′}. In some embodiments, R_{2b′} is butyl optionally substituted with one or more R_{2b′}. In some embodiments, R_{2b′} is pentyl optionally substituted with one or more R_{2b′}. In some embodiments, R_{2b′} is hexyl optionally substituted with one or more R_{2b′}. In some embodiments, R_{2b′} is isopropyl optionally substituted with one or more R_{2b′}. In some embodiments, R_{2b′} is isobutyl optionally substituted with one or more R_{2b′}. In some embodiments, R_{2b′} is isopentyl optionally substituted with one or more R_{2b′}. In some embodiments, R_{2b′} is isohexyl optionally substituted with one or more R_{2b′}. In some embodiments, R_{2b′} is secbutyl optionally substituted with one or more R_{2b′}. In some embodiments, R_{2b′} is secpentyl optionally substituted with one or more R_{2b′}. In some embodiments, R_{2b′} is sechexyl optionally substituted with one or more R_{2b′}. In some embodiments, R_{2b′} is tertbutyl optionally substituted with one or more R_{2b″}.

In some embodiments, R_{2b′} is C₁-C₆ alkyl. In some embodiments, R_{2b″} is methyl. In some embodiments, R_{2b′} is ethyl. In some embodiments, R_{2b″} is propyl. In some embodiments, R_{2b′} is butyl. In some embodiments, R_{2b′} is pentyl. In some embodiments, R_{2b′} is hexyl. In some embodiments, R_{2b′} is isopropyl. In some embodiments, R_{2b′} is isobutyl. In some embodiments, R_{2b′} is isopentyl. In some embodiments, R_{2b′} is isohexyl. In some embodiments, R_{2b′} is secbutyl.

In some embodiments, R_{2b′} is secpentyl. In some embodiments, R_{2b′} is sechexyl. In some embodiments, R_{2b′} is tertbutyl.

In some embodiments, R_{2b′} is C₂-C₆ alkenyl optionally substituted with one or more R_{2b′}.

In some embodiments, R_{2b′} is C₂ alkenyl optionally substituted with one or more R_{2b′}. In some embodiments, R_{2b′} is C₃ alkenyl optionally substituted with one or more R_{2b′}. In some embodiments, R_{2b′} is C₄ alkenyl optionally substituted with one or more R_{2b′}. In some embodiments, R_{2b′} is C₅ alkenyl optionally substituted with one or more R_{2b″}. In some embodiments, R_{2b′} is C₆ alkenyl optionally substituted with one or more R_{2b″}.

In some embodiments, R_{2b′} is C₂-C₆ alkenyl. In some embodiments, R_{2b′} is C₂ alkenyl. In some embodiments, R_{2b′} is C₂ alkenyl. In some embodiments, R_{2b′} is C₄ alkenyl. In some embodiments, R_{2b′} is C₅ alkenyl. In some embodiments, R_{2b′} is C₆ alkenyl.

In some embodiments, R_{2b′} is C₂-C₆ alkynyl optionally substituted with one or more R_{2b′}.

In some embodiments, R_{2b′} is C₂ alkynyl optionally substituted with one or more R_{2b′}. In some embodiments, R_{2b′} is C₃ alkynyl optionally substituted with one or more R_{2b′}. In some embodiments, R_{2b′} is C₄ alkynyl optionally substituted with one or more R_{2b″}. In some embodiments, R_{2b′} is C₅ alkynyl optionally substituted with one or more R_{2b′}. In some embodiments, R_{2b′} is C₆ alkynyl optionally substituted with one or more R_{2b″}.

In some embodiments, R_{2b′} is C₂-C₆ alkynyl. In some embodiments, R_{2b′} is C₂ alkynyl. In some embodiments, R_{2a′} is C₃ alkynyl. In some embodiments, R_{2b′} is C₄ alkynyl. In some embodiments, R_{2b′} is C₅ alkynyl. In some embodiments, R_{2b′} is C₆ alkynyl.

In some embodiments, R_{2b′} is C₁-C₆ haloalkyl optionally substituted with one or more R_{2b″}.

In some embodiments, R_{2b′} is halomethyl optionally substituted with one or more R_{2b″}. In some embodiments, R_{2b′} is haloethyl optionally substituted with one or more R_{2b′}. In some embodiments, R_{2b′} is halopropyl optionally substituted with one or more R_{2b′}. In some embodiments, R_{2b′} is halobutyl optionally substituted with one or more R_{2b′}. In some embodiments, R_{2b′} is halopentyl optionally substituted with one or more R_{2b′}. In some embodiments, R_{2b′} is halohexyl optionally substituted with one or more R_{2b″}.

In some embodiments, R_{2b′} is C₁-C₆ haloalkyl. In some embodiments, R_{2b′} is halomethyl.

In some embodiments, R_{2b′} is haloethyl. In some embodiments, R_{2b′} is halopropyl. In some embodiments, R_{2b′} is halobutyl. In some embodiments, R_{2b′} is halopentyl. In some embodiments, R_{2b′} is halohexyl.

In some embodiments, R_{2b′} is —(CH₂)ₘ˜-OR_{2b″}, —(CH₂)ₘ—N(R_{2b″})(R_{2b′}), —(CH₂)ₘ—C(O)OR_{2b′}, —C(O)R_{2b′}, —C(O)N(R_{2b′})(R_{2b″}), or —N(R_{2b″})C(O)R_{2b″}.

In some embodiments, R_{2b′} is —(CH₂)ₘ—OR_{2b′}, —(CH₂)ₘ—N(R_{2b′})(R_{2b′″}), —C(O)OR_{2b′}, —C(O)R_{2b″}, —C(O)N(R_{2b′})(R_{2b″}), or —N(R_{2b′})C(O)R_{2b′″}.

In some embodiments, R_{2b′} is —(CH₂)ₘ—OR_{2b″} or —(CH₂)ₘ—N(R_{2b′})(R_{2b″}).

In some embodiments, R_{2b′} is —(CH₂)ₘ—OR_{2b″}. In some embodiments, R_{2b′} is —OR_{2b′}. In some embodiments, R_{2b′} is —CH₂—OR_{2b″}. In some embodiments, R_{2b′} is —(CH₂)₂—OR_{2b″}. In some embodiments, R_{2b′} is —(CH₂)₃—OR_{2b″}. In some embodiments, R_{2b′} is —(CH₂)₄—OR_{2b″}.

In some embodiments, R_{2b′} is —(CH₂)ₘ—N(R_{2b′})(R_{2b″}). In some embodiments, R_{2b′} is —N(R_{2b′})(R_{2b″}). In some embodiments, R_{2b′} is —CH₂—N(R_{2b′})(R_{2b″}). In some embodiments, R_{2b′} is —(CH₂)₂—N(R_{2b″})(R_{2b′″}). In some embodiments, R_{2b′} is —(CH₂)₃—N(R_{2b′})(R_{2b′″}). In some embodiments, R_{2b′} is —(CH₂)₄—N(R_{2b″})(R_{2b′″}).

In some embodiments, R_{2b′} is —C(O)OR_{2b′}, —C(O)R_{2b″}, —C(O)N(R_{2b″})(R_{2b′″}), or —N(R_{2b′})C(O)R_{2b″}.

In some embodiments, R_{2b″} is —(CH₂)ₘ—C(O)OR_{2b″}. In some embodiments, R_{2b′} is —C(O)OR_{2b″}. In some embodiments, R_{2b′} is —(CH₂)₁—C(O)OR_{2b″}. In some embodiments, R_{2b′} is —(CH₂)₂—C(O)OR_{2b″}. In some embodiments, R_{2b′} is —(CH₂)₃—C(O)OR_{2b″}. In some embodiments, R_{2b′} is —(CH₂)₄—C(O)OR_{2b″}.

In some embodiments, R_{2b′} is —C(O)R_{2b″}. In some embodiments, R_{2b′} is —C(O)N(R_{2b″})(R_{2b″}). In some embodiments, R_{2b′} is —N(R_{2b″})C(O)R_{2b′}.

In some embodiments, R_{2b′} is C₃-C₇ cycloalkyl 3- to 10-membered heterocyclyl, or 5- to 10-membered heteroaryl, wherein the cycloalkyl or heterocyclyl is optionally substituted with one or more R_{2b″}.

In some embodiments, R_{2b′} is C₃-C₇ cycloalkyl or 3- to 10-membered heterocyclyl, wherein the cycloalkyl or heterocyclyl is optionally substituted with one or more R_{2b″}.

In some embodiments, R_{2b′} is C₃-C₇ cycloalkyl or 3- to 10-membered heterocyclyl.

In some embodiments, R_{2b′} is C₃-C₇ cycloalkyl optionally substituted with one or more R_{2b″}. In some embodiments, R_{2b′} is C₃ cycloalkyl optionally substituted with one or more R_{2b″}. In some embodiments, R_{2b′} is C₄ cycloalkyl optionally substituted with one or more R_{2b″}. In some embodiments, R_{2b′} is C₅ cycloalkyl optionally substituted with one or more R_{2b″}. In some embodiments, R_{2b′} is C₆ cycloalkyl optionally substituted with one or more R_{2b″}. In some embodiments, R_{2b′} is C₇ cycloalkyl optionally substituted with one or more R_{2b″}.

In some embodiments, R_{2b′} is C₃-C₇ cycloalkyl. In some embodiments, R_{2b′} is C₃ cycloalkyl. In some embodiments, R_{2b′} is C₄ cycloalkyl. In some embodiments, R_{2b′} is C₅ cycloalkyl. In some embodiments, R_{2b′} is C₆ cycloalkyl. In some embodiments, R_{2b′} is C₇ cycloalkyl.

In some embodiments, R_{2b′} is 3- to 10-membered heterocyclyl optionally substituted with one or more R_{2b′}. In some embodiments, R_{2b′} is 3-membered heterocyclyl optionally substituted with one or more R_{2b′}. In some embodiments, R_{2b′} is 4-membered heterocyclyl optionally substituted with one or more R_{2b′}. In some embodiments, R_{2b′} is 5-membered heterocyclyl optionally substituted with one or more R_{2b″}. In some embodiments, R_{2b′} is 6-membered heterocyclyl optionally substituted with one or more R_{2b″}. In some embodiments, R_{2b′} is 7-membered heterocyclyl optionally substituted with one or more R_{2b′}. In some embodiments, R_{2b′} is 8-membered heterocyclyl optionally substituted with one or more R_{2b′}. In some embodiments, R_{2b′} is 9-membered heterocyclyl optionally substituted with one or more R_{2b″}. In some embodiments, R_{2b′} is 10-membered heterocyclyl optionally substituted with one or more R_{2b′}.

In some embodiments, R_{2b′} is 3- to 10-membered heterocyclyl. In some embodiments, R_{2b′} is 3-membered heterocyclyl. In some embodiments, R_{2b′} is 4-membered heterocyclyl. In some embodiments, R_{2b′} is 5-membered heterocyclyl. In some embodiments, R_{2b′} is 6-membered heterocyclyl. In some embodiments, R_{2b′} is 7-membered heterocyclyl. In some embodiments, R_{2b′} is 8-membered heterocyclyl. In some embodiments, R_{2b′} is 9-membered heterocyclyl. In some embodiments, R_{2b′} is 10-membered heterocyclyl.

In some embodiments, R_{2b′} is 5- to 10-membered heteroaryl. In some embodiments, R_{2b′} is 5-membered heteroaryl. In some embodiments, R_{2b′} is 6-membered heteroaryl. In some embodiments, R_{2b″} is 7-membered heteroaryl. In some embodiments, R_{2b′} is 8-membered heteroaryl. In some embodiments, R_{2b′} is 9-membered heteroaryl. In some embodiments, R_{2b′} is 10-membered heteroaryl.

In some embodiments, R_{2b′} is C₁-C₆ alkyl, —(CH₂)ₘ—OR_{2b″}, —(CH₂)ₘ—N(R_{2b′})(R_{2b′″}), —C(O)OR_{2b′}, —C(O)R_{2b″}, —C(O)N(R_{2b′})(R_{2b′}), —N(R_{2b″})C(O)R_{2b′″}, C₃-C₇ cycloalkyl, or 3- to 10-membered heterocyclyl, wherein the alkyl, cycloalkyl, or heterocyclyl is optionally substituted with one or more R_{2b″}.

In some embodiments, R_{2b″} is H, oxo, —CN, —OH, C₁-C₆ alkyl, C₂-C₆ alkenyl, C₂-C₆ alkynyl, or C₁-C₆ haloalkyl, wherein the alkyl, alkenyl, or alkynyl is optionally substituted with 3- to 10-membered heterocyclyl, wherein the heterocyclyl is optionally substituted with oxo.

In some embodiments, R_{2b″} is H, oxo, —CN, —OH, C₁-C₆ alkyl, C₂-C₆ alkenyl, C₂-C₆ alkynyl, or C₁-C₆ haloalkyl, wherein the alkyl, alkenyl, or alkynyl is optionally substituted with 3- to 10-membered heterocyclyl.

In some embodiments, R_{2b″} is H, oxo, —CN, —OH, C₁-C₆ alkyl, C₂-C₆ alkenyl, C₂-C₆ alkynyl, or C₁-C₆ haloalkyl.

In some embodiments, R_{2b″} is H.

In some embodiments, R_{2b″} is oxo, —CN, —OH, C₁-C₆ alkyl, C₂-C₆ alkenyl, C₂-C₆ alkynyl, or C₁-C₆ haloalkyl, wherein the alkyl, alkenyl, or alkynyl is optionally substituted with 3- to 10-membered heterocyclyl, wherein the heterocyclyl is optionally substituted with oxo.

In some embodiments, R_{2b″} is oxo, —CN, —OH, C₁-C₆ alkyl, C₂-C₆ alkenyl, C₂-C₆ alkynyl, or C₁-C₆ haloalkyl, wherein the alkyl, alkenyl, or alkynyl is optionally substituted with 3- to 10-membered heterocyclyl.

In some embodiments, R_{2b″} is oxo, —CN, —OH, C₁-C₆ alkyl, C₂-C₆ alkenyl, C₂-C₆ alkynyl, or C₁-C₆ haloalkyl.

In some embodiments, R_{2b″} is oxo, —CN, —OH.

In some embodiments, R_{2b″} is oxo. In some embodiments, R_{2b″} is —CN. In some embodiments, R_{2b″} is —OH.

In some embodiments, R_{2b″} is C₁-C₆ alkyl, C₂-C₆ alkenyl, C₂-C₆ alkynyl, or C₁-C₆ haloalkyl, wherein the alkyl, alkenyl, or alkynyl is optionally substituted with 3- to 10-membered heterocyclyl, wherein the heterocyclyl is optionally substituted with oxo.

In some embodiments, R_{2b″} is C₁-C₆ alkyl, C₂-C₆ alkenyl, C₂-C₆ alkynyl, or C₁-C₆ haloalkyl.

In some embodiments, R_{2b″} is C₁-C₆ alkyl, C₂-C₆ alkenyl, or C₂-C₆ alkynyl, wherein the alkyl, alkenyl, or alkynyl is optionally substituted with 3- to 10-membered heterocyclyl, wherein the heterocyclyl is optionally substituted with oxo.

In some embodiments, R_{2b″} is C₁-C₆ alkyl, C₂-C₆ alkenyl, or C₂-C₆ alkynyl.

In some embodiments, R_{2b″} is C₁-C₆ alkyl, wherein the alkyl is optionally substituted with 3- to 10-membered heterocyclyl, wherein the heterocyclyl is optionally substituted with oxo. In some embodiments, R_{2b″} is methyl, wherein the alkyl is optionally substituted with 3- to 10-membered heterocyclyl, wherein the heterocyclyl is optionally substituted with oxo. In some embodiments, R_{2b″} is ethyl, wherein the alkyl is optionally substituted with 3- to 10-membered heterocyclyl, wherein the heterocyclyl is optionally substituted with oxo. In some embodiments, R_{2b″} is propyl, wherein the alkyl is optionally substituted with 3- to 10-membered heterocyclyl, wherein the heterocyclyl is optionally substituted with oxo. In some embodiments, R_{2b″} is butyl, wherein the alkyl is optionally substituted with 3- to 10-membered heterocyclyl, wherein the heterocyclyl is optionally substituted with oxo. In some embodiments, R_{2b″} is pentyl, wherein the alkyl is optionally substituted with 3- to 10-membered heterocyclyl, wherein the heterocyclyl is optionally substituted with oxo. In some embodiments, R_{2b″} is hexyl, wherein the alkyl is optionally substituted with 3- to 10-membered heterocyclyl, wherein the heterocyclyl is optionally substituted with oxo. In some embodiments, R_{2b″} is isopropyl, wherein the alkyl is optionally substituted with 3- to 10-membered heterocyclyl, wherein the heterocyclyl is optionally substituted with oxo. In some embodiments, R_{2b″} is isobutyl, wherein the alkyl is optionally substituted with 3- to 10-membered heterocyclyl, wherein the heterocyclyl is optionally substituted with oxo. In some embodiments, R_{2b″} is isopentyl, wherein the alkyl is optionally substituted with 3- to 10-membered heterocyclyl, wherein the heterocyclyl is optionally substituted with oxo. In some embodiments, R_{2b″} is isohexyl, wherein the alkyl is optionally substituted with 3- to 10-membered heterocyclyl, wherein the heterocyclyl is optionally substituted with oxo. In some embodiments, R_{2b″} is secbutyl, wherein the alkyl is optionally substituted with 3- to 10-membered heterocyclyl, wherein the heterocyclyl is optionally substituted with oxo. In some embodiments, R_{2b″} is secpentyl, wherein the alkyl is optionally substituted with 3- to 10-membered heterocyclyl, wherein the heterocyclyl is optionally substituted with oxo. In some embodiments, R_{2b″} is sechexyl, wherein the alkyl is optionally substituted with 3- to 10-membered heterocyclyl, wherein the heterocyclyl is optionally substituted with oxo. In some embodiments, R_{2b″} is tertbutyl, wherein the alkyl is optionally substituted with 3- to 10-membered heterocyclyl, wherein the heterocyclyl is optionally substituted with oxo.

In some embodiments, R_{2b″} is C₁-C₆ alkyl. In some embodiments, R_{2b″} is methyl. In some embodiments, R_{2b″} is ethyl. In some embodiments, R_{2b″} is propyl. In some embodiments, R_{2b″} is butyl. In some embodiments, R_{2b″} is pentyl. In some embodiments, R_{2b″} is hexyl. In some embodiments, R_{2b″} is isopropyl. In some embodiments, R_{2b″} is isobutyl. In some embodiments, R_{2b″} is isopentyl. In some embodiments, R_{2b″} is isohexyl. In some embodiments, R_{2b″} is secbutyl.

In some embodiments, R_{2b″} is secpentyl. In some embodiments, R_{2b″} is sechexyl. In some embodiments, R_{2b″} is tertbutyl.

In some embodiments, R_{2b″} is C₂-C₆ alkenyl, wherein the alkenyl is optionally substituted with 3- to 10-membered heterocyclyl, wherein the heterocyclyl is optionally substituted with oxo.

In some embodiments, R_{2b″} is C₂ alkenyl, wherein the alkenyl is optionally substituted with 3- to 10-membered heterocyclyl, wherein the heterocyclyl is optionally substituted with oxo. In some embodiments, R_{2b″} is C₃ alkenyl, wherein the alkenyl is optionally substituted with 3- to 10-membered heterocyclyl, wherein the heterocyclyl is optionally substituted with oxo. In some embodiments, R_{2b″} is C₄ alkenyl, wherein the alkenyl is optionally substituted with 3- to 10-membered heterocyclyl, wherein the heterocyclyl is optionally substituted with oxo. In some embodiments, R_{2b″} is C₅ alkenyl, wherein the alkenyl is optionally substituted with 3- to 10-membered heterocyclyl, wherein the heterocyclyl is optionally substituted with oxo. In some embodiments, R_{2b″} is C₆ alkenyl, wherein the alkenyl is optionally substituted with 3- to 10-membered heterocyclyl, wherein the heterocyclyl is optionally substituted with oxo.

In some embodiments, R_{2b″} is C₂-C₆ alkenyl. In some embodiments, R_{2b″} is C₂ alkenyl. In some embodiments, R_{2b″} is C₃ alkenyl. In some embodiments, R_{2b″} is C₄ alkenyl. In some embodiments, R_{2b″} is C₅ alkenyl. In some embodiments, R_{2b″} is C₆ alkenyl.

In some embodiments, R_{2b″} is C₂-C₆ alkynyl, wherein the alkynyl is optionally substituted with 3- to 10-membered heterocyclyl, wherein the heterocyclyl is optionally substituted with oxo.

In some embodiments, R_{2b″} is C₂ alkynyl, wherein the alkynyl is optionally substituted with 3- to 10-membered heterocyclyl, wherein the heterocyclyl is optionally substituted with oxo. In some embodiments, R_{2b″} is C₃ alkynyl, wherein the alkynyl is optionally substituted with 3- to 10-membered heterocyclyl, wherein the heterocyclyl is optionally substituted with oxo. In some embodiments, R_{2b″} is C₄ alkynyl, wherein the alkynyl is optionally substituted with 3- to 10-membered heterocyclyl, wherein the heterocyclyl is optionally substituted with oxo. In some embodiments, R_{2b″} is C₅ alkynyl, wherein the alkynyl is optionally substituted with 3- to 10-membered heterocyclyl, wherein the heterocyclyl is optionally substituted with oxo. In some embodiments, R_{2b″} is C₆ alkynyl, wherein the alkynyl is optionally substituted with 3- to 10-membered heterocyclyl, wherein the heterocyclyl is optionally substituted with oxo.

In some embodiments, R_{2b″} is C₂-C₆ alkynyl. In some embodiments, R_{2b″} is C₂ alkynyl. In some embodiments, R_{2b″} is C₃ alkynyl. In some embodiments, R_{2b″} is C₄ alkynyl. In some embodiments, R_{2b″} is C₅ alkynyl. In some embodiments, R_{2b″} is C₆ alkynyl.

In some embodiments, R_{2b″} is C₁-C₆ haloalkyl, wherein the alkyl is optionally substituted with 3- to 10-membered heterocyclyl, wherein the heterocyclyl is optionally substituted with oxo.

In some embodiments, R_{2b″} is halomethyl, wherein the alkyl is optionally substituted with 3- to 10-membered heterocyclyl, wherein the heterocyclyl is optionally substituted with oxo. In some embodiments, R_{2b″} is haloethyl, wherein the alkyl is optionally substituted with 3- to 10-membered heterocyclyl, wherein the heterocyclyl is optionally substituted with oxo. In some embodiments, R_{2b″} is halopropyl, wherein the alkyl is optionally substituted with 3- to 10-membered heterocyclyl, wherein the heterocyclyl is optionally substituted with oxo. In some embodiments, R_{2b″} is halobutyl, wherein the alkyl is optionally substituted with 3- to 10-membered heterocyclyl, wherein the heterocyclyl is optionally substituted with oxo. In some embodiments, R_{2b″} is halopentyl, wherein the alkyl is optionally substituted with 3- to 10-membered heterocyclyl, wherein the heterocyclyl is optionally substituted with oxo. In some embodiments, R_{2b″} is halohexyl, wherein the alkyl is optionally substituted with 3- to 10-membered heterocyclyl, wherein the heterocyclyl is optionally substituted with oxo.

In some embodiments, R_{2b″} is C₁-C₆ haloalkyl. In some embodiments, R_{2b″} is halomethyl.

In some embodiments, R_{2b″} is haloethyl. In some embodiments, R_{2b″} is halopropyl. In some embodiments, R_{2b″} is halobutyl. In some embodiments, R_{2b″} is halopentyl. In some embodiments, R_{2b″} is halohexyl.

In some embodiments, R_{2b″} is H, oxo, —CN, —OH, C₁-C₆ alkyl, wherein the alkyl is optionally substituted with 3- to 10-membered heterocyclyl, wherein the heterocyclyl is optionally substituted with oxo.

In some embodiments, R_{2b″} is H, C₁-C₆ alkyl optionally substituted with one or more —C(O)OH, —OH or —NH₂, C₂-C₆ alkenyl, C₂-C₆ alkynyl, C₃-C₇ cycloalkyl, or —C(O)C₁-C₆ alkyl.

In some embodiments, R_{2b″} is H, C₁-C₆ alkyl, C₂-C₆ alkenyl, C₂-C₆ alkynyl, C₃-C₇ cycloalkyl, or —C(O)C₁-C₆ alkyl.

In some embodiments, R_{2b″} is H.

In some embodiments, R_{2b′″} is C₁-C₆ alkyl optionally substituted with one or more —C(O)OH, —OH or —NH₂, C₂-C₆ alkenyl, C₂-C₆ alkynyl, C₃-C₇ cycloalkyl, or —C(O)C₁-C₆ alkyl.

In some embodiments, R_{2b′″} is C₁-C₆ alkyl, C₂-C₆ alkenyl, C₂-C₆ alkynyl, C₃-C₇ cycloalkyl, or —C(O)C₁-C₆ alkyl.

In some embodiments, R_{2b′″} is C₁-C₆ alkyl optionally substituted with one or more —C(O)OH, —OH or —NH₂, C₂-C₆ alkenyl, or C₂-C₆ alkynyl.

In some embodiments, R_{2b′″} is C₁-C₆ alkyl, C₂-C₆ alkenyl, or C₂-C₆ alkynyl.

In some embodiments, R_{2b″} is C₁-C₆ alkyl optionally substituted with one or more —C(O)OH, —OH or —NH₂. In some embodiments, R_{2b″} is methyl optionally substituted with one or more —C(O)OH, —OH or —NH₂. In some embodiments, R_{2b′″} is ethyl optionally substituted with one or more —C(O)OH, —OH or —NH₂. In some embodiments, R_{2b′″} is propyl optionally substituted with one or more —C(O)OH, —OH or —NH₂. In some embodiments, R_{2b′″} is butyl optionally substituted with one or more —C(O)OH, —OH or —NH₂. In some embodiments, R_{2b′″}, is pentyl optionally substituted with one or more —C(O)OH, —OH or —NH₂. In some embodiments, R_{2b″} is hexyl optionally substituted with one or more —C(O)OH, —OH or —NH₂. In some embodiments, R_{2b″} is isopropyl optionally substituted with one or more —C(O)OH, —OH or —NH₂. In some embodiments, R_{2b′″} is isobutyl optionally substituted with one or more —C(O)OH, —OH or —NH₂. In some embodiments, R_{2b′″} is isopentyl optionally substituted with one or more —C(O)OH, —OH or —NH₂. In some embodiments, R_{2b′″} is isohexyl optionally substituted with one or more —C(O)OH, —OH or —NH₂. In some embodiments, R_{2b′″} is secbutyl optionally substituted with one or more —C(O)OH, —OH or —NH₂. In some embodiments, R_{2b″} is secpentyl optionally substituted with one or more —C(O)OH, —OH or —NH₂. In some embodiments, R_{2b′″} is sechexyl optionally substituted with one or more —C(O)OH, —OH or —NH₂. In some embodiments, R_{2b′″} is tertbutyl optionally substituted with one or more —C(O)OH, —OH or —NH₂.

In some embodiments, R_{2b′″} is C₁-C₆ alkyl optionally substituted with one or more —C(O)OH. In some embodiments, R_{2b′″} is C₁-C₆ alkyl optionally substituted with one or more —OH. In some embodiments, R_{2b′″} is C₁-C₆ alkyl optionally substituted with one or more —NH₂.

In some embodiments, R_{2b′″} is C₁-C₆ alkyl substituted with —C(O)OH. In some embodiments, R_{2b} is C₁-C₆ alkyl substituted with —OH. In some embodiments, R_{2b′″} is C₁-C₆ alkyl substituted with —NH₂.

In some embodiments, R_{2b′″} is C₁-C₆ alkyl. In some embodiments, R_{2b′″}, is methyl. In some embodiments, R_{2b′″} is ethyl. In some embodiments, R_{2b′″} is propyl. In some embodiments, R_{2b′″} is butyl. In some embodiments, R_{2b′″} is pentyl. In some embodiments, R_{2b′″} is hexyl. In some embodiments, R_{2b′″} is isopropyl. In some embodiments, R_{2b′″} is isobutyl. In some embodiments, R_{2b′″} is isopentyl. In some embodiments, R_{2b′″} is isohexyl. In some embodiments, R_{2b′″} is secbutyl.

In some embodiments, R_{2b′″} is secpentyl. In some embodiments, R_{2b′″} is sechexyl. In some embodiments, R_{2b′″} is tertbutyl.

In some embodiments, R_{2b′″} is C₂-C₆ alkenyl. In some embodiments, R_{2b′″} is C₂ alkenyl. In some embodiments, R_{2b′″} is C₃ alkenyl. In some embodiments, R_{2b′″} is C₄ alkenyl. In some embodiments, R_{2b′″}, is C₅ alkenyl. In some embodiments, R_{2b′″} is C₆ alkenyl. In some embodiments, R_{2b″} is C₂-C₆ alkynyl. In some embodiments, R_{2b′″} is C₂ alkynyl. In some embodiments, R_{2b′″} is C₃ alkynyl. In some embodiments, R_{2b′″} is C₄ alkynyl. In some embodiments, R_{2b′″} is C₅ alkynyl. In some embodiments, R_{2b′″} is C, alkynyl.

In some embodiments, R_{2b} is —C(O)C₁-C₆ alkyl. In some embodiments, R_{2b′″} is —C(O)C₁ alkyl. In some embodiments, R_{2b′″} is —C(O)C₂ alkyl. In some embodiments, R_{2b′″} is —C(O)C₃ alkyl.

In some embodiments, R_{2b′″} is —C(O)C₄ alkyl. In some embodiments, R_{2b′″} is —C(O)C₅ alkyl. In some embodiments, R_{2b′″} is —C(O)C₆ alkyl.

In some embodiments, R_{2b′″} is H, C₁-C₆ alkyl, C₃-C₇ cycloalkyl, or —C(O)C₁-C₆ alkyl.

In some embodiments, each R₄ or R₅ is independently H, —O—C₁-C₆alkyl, —NH—C₁-C₆alkyl, —NH-(5- to 10-membered heteroaryl), —NH—(C₆-C₁₀ aryl), —NH—C(O)Ra, C₁-C₆ alkyl, C₂-C₆ alkenyl, C₂-C₆ alkynyl, C₁-C₆ haloalkyl, C₆-C₁₀ aryl, or 5- to 10-membered heteroaryl, wherein the alkyl, alkenyl, alkynyl, aryl, or heteroaryl is optionally substituted with one or more R₅ₐ₁.

In some embodiments, R₄ is H, —O—C₁-C₆ alkyl, —NH—C₁-C₆ alkyl, —NH-(5- to 10-membered heteroaryl), —NH—(C₆-C₁₀ aryl), —NH—C(O)R₅ₐ, C₁-C₆ alkyl, C₂-C₆ alkenyl, C₂-C₆ alkynyl, C₁-C₆ haloalkyl, C₆-C₁₀ aryl, or 5- to 10-membered heteroaryl, wherein the alkyl, alkenyl, alkynyl, aryl, or heteroaryl is optionally substituted with one or more R₅ₐ₁.

In some embodiments, R₄ is H, —O—C₁-C₆ alkyl, —NH—C₁-C₆ alkyl, —NH-(5- to 10-membered heteroaryl), —NH—(C₆-C₁₀ aryl), —NH—C(O)R₅ₐ, C₁-C₆ alkyl, C₂-C₆ alkenyl, C₂-C₆ alkynyl, C₁-C₆ haloalkyl, C₆-C₁₀ aryl, or 5- to 10-membered heteroaryl, wherein the alkyl, alkenyl, alkynyl, aryl, or heteroaryl is optionally substituted with one or more R₅ₐ₁.

In some embodiments, R₄ is H.

In some embodiments, R₄ is —O—C₁-C₆ alkyl, —NH—C₁-C₆ alkyl, —NH-(5- to 10-membered heteroaryl), —NH—(C₆-C₁₀ aryl), —NH—C(O)R₅ₐ, C₁-C₆ alkyl, C₂-C₆ alkenyl, C₂-C₆ alkynyl, C₁-C₆ haloalkyl, C₆-C₁₀ aryl, or 5- to 10-membered heteroaryl, wherein the alkyl, alkenyl, alkynyl, aryl, or heteroaryl is optionally substituted with one or more R₅ₐ₁.

In some embodiments, R₄ is —O—C₁-C₆ alkyl, —NH—C₁-C₆ alkyl, —NH-(5- to 10-membered heteroaryl), —NH—(C₆-C₁₀ aryl), —NH—C(O)R₅ₐ, C₁-C₆ alkyl, C₂-C₆ alkenyl, C₂-C₆ alkynyl, C₁-C₆ haloalkyl, C₆-C₁₀ aryl, or 5- to 10-membered heteroaryl.

In some embodiments, R₄ is —O—C₁-C₆ alkyl, —NH—C₁-C₆ alkyl, —NH-(5- to 10-membered heteroaryl), —NH—(C₆-C₁₀ aryl), —NH—C(O)R₅ₐ, wherein the alkyl, aryl, or heteroaryl is optionally substituted with one or more R₅ₐ₁.

In some embodiments, R₄ is —O—C₁-C₆ alkyl, —NH—C₁-C₆ alkyl, —NH-(5- to 10-membered heteroaryl), —NH—(C₆-C₁₀ aryl), —NH—C(O)R₅ₐ.

In some embodiments, R₄ is —O—C₁-C₆ alkyl, wherein the alkyl is optionally substituted with one or more R₅ₐ₁. In some embodiments, R₄ is —O—C₁-C₆ alkyl.

In some embodiments, R₄ is —O—C₁ alkyl, wherein the alkyl is optionally substituted with one or more R₅ₐ₁. In some embodiments, R₄ is —O—C₁ alkyl.

In some embodiments, R₄ is —O—C₂ alkyl, wherein the alkyl is optionally substituted with one or more R₅ₐ₁. In some embodiments, R₄ is —O—C₂alkyl.

In some embodiments, R₄ is —O—C₃ alkyl, wherein the alkyl is optionally substituted with one or more R₅ₐ₁. In some embodiments, R₄ is —O—C₃ alkyl.

In some embodiments, R₄ is —O—C₄ alkyl, wherein the alkyl is optionally substituted with one or more R₅ₐ₁. In some embodiments, R₄ is —O—C₄ alkyl.

In some embodiments, R₄ is —O—C₈ alkyl, wherein the alkyl is optionally substituted with one or more R₅ₐ₁. In some embodiments, R₄ is —O—C₈ alkyl.

In some embodiments, R₄ is —O—C₆ alkyl, wherein the alkyl is optionally substituted with one or more R₅ₐ₁. In some embodiments, R₄ is —O—C₆alkyl.

In some embodiments, R₄ is —NH—C₁-C₆alkyl, —NH-(5- to 10-membered heteroaryl), —NH—(C₆-C₁₀ aryl), —NH—C(O)R₅ₐ, wherein the alkyl, aryl, or heteroaryl is optionally substituted with one or more R₅ₐ₁.

In some embodiments, R₄ is —NH—C₁-C₆alkyl, —NH-(5- to 10-membered heteroaryl), —NH—(C₆-C₁₀ aryl), —NH—C(O)R₁ₐ.

In some embodiments, R₄ is —NH—C₁-C₆ alkyl, wherein the alkyl is optionally substituted with one or more R₅ₐ₁. In some embodiments, R₄ is —NH—C₁-C₆ alkyl.

In some embodiments, R₄ is —NH—C₁ alkyl, wherein the alkyl is optionally substituted with one or more R₅ₐ₁. In some embodiments, R₄ is —NH—C₁ alkyl.

In some embodiments, R₄ is —NH—C₂ alkyl, wherein the alkyl is optionally substituted with one or more R₅ₐ₁. In some embodiments, R₄ is —NH—C₂alkyl.

In some embodiments, R₄ is —NH—C₃ alkyl, wherein the alkyl is optionally substituted with one or more R₅ₐ₁. In some embodiments, R₄ is —NH—C₇ alkyl.

In some embodiments, R₄ is —NH—C₄ alkyl, wherein the alkyl is optionally substituted with one or more R₅ₐ₁. In some embodiments, R₄ is —NH—C₄ alkyl.

In some embodiments, R₄ is —NH—C₅ alkyl, wherein the alkyl is optionally substituted with one or more R₅ₐ₁. In some embodiments, R₄ is —NH—C₈ alkyl.

In some embodiments, R₄ is —NH—C₆ alkyl, wherein the alkyl is optionally substituted with one or more R₅ₐ₁. In some embodiments, R₄ is —NH—C₆ alkyl.

In some embodiments, R₄ is —NH-(5- to 10-membered heteroaryl), wherein the heteroaryl is optionally substituted with one or more R₅ₐ₁.

In some embodiments, R₄ is —NH-(5- to 10-membered heteroaryl).

In some embodiments, R₄ is —NH-(10-membered heteroaryl), wherein the heteroaryl is optionally substituted with one or more R₅ₐ₁. In some embodiments, R₄ is —NH-(10-membered heteroaryl).

In some embodiments, R₄ is —NH-(9-membered heteroaryl), wherein the heteroaryl is optionally substituted with one or more R₅ₐ₁. In some embodiments, R₄ is —NH-(9-membered heteroaryl).

In some embodiments, R₄ is —NH-(8-membered heteroaryl), wherein the heteroaryl is optionally substituted with one or more R₅ₐ₁. In some embodiments, R₄ is —NH-(8-membered heteroaryl).

In some embodiments, R₄ is —NH-(7-membered heteroaryl), wherein the heteroaryl is optionally substituted with one or more R₅ₐ₁. In some embodiments, R₄ is —NH-(7-membered heteroaryl).

In some embodiments, R₄ is —NH-(6-membered heteroaryl), wherein the heteroaryl is optionally substituted with one or more R₅ₐ₁. In some embodiments, R₄ is —NH-(6-membered heteroaryl).

In some embodiments, R₄ is —NH-(5-membered heteroaryl), wherein the heteroaryl is optionally substituted with one or more R₅ₐ₁. In some embodiments, R₄ is —NH-(5-membered heteroaryl).

In some embodiments, R₄ is —NH—(C₆-C₁₀ aryl), wherein the aryl is optionally substituted with one or more R₅ₐ₁. In some embodiments, R₄ is —NH—(C₆-C₁₀ aryl).

In some embodiments, R₄ is —NH—(C₆ aryl), wherein the aryl is optionally substituted with one or more R₅ₐ₁. In some embodiments, R₄ is —NH—(C₆ aryl).

In some embodiments, R₄ is —NH—(C₈ aryl), wherein the aryl is optionally substituted with one or more R₅ₐ₁. In some embodiments, R₄ is —NH—(C₅ aryl).

In some embodiments, R₄ is —NH—(C₁₀ aryl), wherein the aryl is optionally substituted with one or more R₅ₐ₁. In some embodiments, R₄ is —NH—(C₁₀ aryl).

In some embodiments, R₄ is —NH—C(O)R₅ₐ.

In some embodiments, R₄ is C₁-C₆ alkyl, C₂-C₆ alkenyl, C₂-C₆ alkynyl, or C₁-C₆ haloalkyl, wherein the alkyl, alkenyl, or alkynyl is optionally substituted with one or more R₅ₐ₁.

In some embodiments, R₄ is C₁-C₆ alkyl, C₂-C₆ alkenyl, C₂-C₆ alkynyl, or C₁-C₆ haloalkyl.

In some embodiments, R₄ is C₁-C₆ alkyl, C₂-C₆ alkenyl, or C₂-C₆ alkynyl, wherein the alkyl, alkenyl, or alkynyl is optionally substituted with one or more R₅ₐ₁.

In some embodiments, R₄ is C₁-C₆ alkyl, C₂-C₆ alkenyl, or C₂-C₆ alkynyl.

In some embodiments, R₄ is C₁-C₆ alkyl optionally substituted with one or more R₅ₐ₁. In some embodiments, R₄ is methyl optionally substituted with one or more R₅ₐ₁. In some embodiments, R₄ is ethyl optionally substituted with one or more R₅ₐ₁. In some embodiments, R₄ is propyl optionally substituted with one or more R₅ₐ₁. In some embodiments, R₄ is butyl optionally substituted with one or more R₅ₐ₁. In some embodiments, R₄ is pentyl optionally substituted with one or more R₅ₐ₁. In some embodiments, R₄ is hexyl optionally substituted with one or more R₅ₐ₁.

In some embodiments, R₄ is isopropyl optionally substituted with one or more R₅ₐ₁. In some embodiments, R₄ is isobutyl optionally substituted with one or more R₅ₐ₁. In some embodiments, R₄ is isopentyl optionally substituted with one or more R₅ₐ₁. In some embodiments, R₄ is isohexyl optionally substituted with one or more R₅ₐ₁. In some embodiments, R₄ is secbutyl optionally substituted with one or more R₅ₐ₁. In some embodiments, R₄ is secpentyl optionally substituted with one or more R₅ₐ₁. In some embodiments, R₄ is sechexyl optionally substituted with one or more R₅ₐ₁. In some embodiments, R₄ is tertbutyl optionally substituted with one or more R₅ₐ₁.

In some embodiments, R₄ is C₁-C₆ alkyl. In some embodiments, R₄ is methyl. In some embodiments, R₄ is ethyl. In some embodiments, R₄ is propyl. In some embodiments, R₄ is butyl. In some embodiments, R₄ is pentyl. In some embodiments, R₄ is hexyl. In some embodiments, R₄ is isopropyl. In some embodiments, R₄ is isobutyl. In some embodiments, R₄ is isopentyl. In some embodiments, R₄ is isohexyl. In some embodiments, R₄ is secbutyl. In some embodiments, R₄ is secpentyl. In some embodiments, R₄ is sechexyl. In some embodiments, R₄ is tertbutyl.

In some embodiments, R₄ is C₂-C₆ alkenyl optionally substituted with one or more R₅ₐ₁. In some embodiments, R₄ is C₂ alkenyl optionally substituted with one or more R₅ₐ. In some embodiments, R₄ is C₃ alkenyl optionally substituted with one or more R₅ₐ₁. In some embodiments, R₄ is C₄ alkenyl optionally substituted with one or more R₅ₐ₁. In some embodiments, R₄ is C₅ alkenyl optionally substituted with one or more R₅ₐ₁. In some embodiments, R₄ is C₆ alkenyl optionally substituted with one or more R₅ₐ₁.

In some embodiments, R₄ is C₂-C₆ alkenyl. In some embodiments, R₄ is C₂ alkenyl. In some embodiments, R₄ is C₃ alkenyl. In some embodiments, R₄ is C₄ alkenyl. In some embodiments, R₄ is C₅ alkenyl. In some embodiments, R₄ is C₆ alkenyl.

In some embodiments, R₄ is C₂-C₆ alkynyl optionally substituted with one or more R₅ₐ₁.

In some embodiments, R₄ is C₂ alkynyl optionally substituted with one or more R₅ₐ₁. In some embodiments, R₄ is C₃ alkynyl optionally substituted with one or more R₅ₐ₁. In some embodiments, R₄ is C₄ alkynyl optionally substituted with one or more R₅ₐ₁. In some embodiments, R₄ is C₅ alkynyl optionally substituted with one or more R₅ₐ₁. In some embodiments, R₄ is C₆ alkynyl optionally substituted with one or more R₅ₐ₁.

In some embodiments, R₄ is C₂-C₆ alkynyl. In some embodiments, R₄ is C₂ alkynyl. In some embodiments, R₄ is C₃ alkynyl. In some embodiments, R₄ is C₄ alkynyl. In some embodiments, R₄ is C₅ alkynyl. In some embodiments, R₄ is C₆ alkynyl.

In some embodiments, R₄ is C₁-C₆ haloalkyl, wherein the alkyl is optionally substituted with one or more R₅ₐ₁. In some embodiments, R₄ is halomethyl, wherein the alkyl is optionally substituted with one or more R₅ₐ₁. In some embodiments, R₄ is haloethyl, wherein the alkyl is optionally substituted with one or more R₅ₐ₁. In some embodiments, R₄ is halopropyl, wherein the alkyl is optionally substituted with one or more R₅ₐ₁. In some embodiments, R₄ is halobutyl, wherein the alkyl is optionally substituted with one or more R₅ₐ₁. In some embodiments, R₄ is halopentyl, wherein the alkyl is optionally substituted with one or more R₅ₐ₁. In some embodiments, R₄ is halohexyl, wherein the alkyl is optionally substituted with one or more R₅ₐ₁.

In some embodiments, R₄ is C₁-C₆ haloalkyl. In some embodiments, R₄ is halomethyl. In some embodiments, R₄ is haloethyl. In some embodiments, R₄ is halopropyl. In some embodiments, R₄ is halobutyl. In some embodiments, R₄ is halopentyl. In some embodiments, R₄ is halohexyl.

In some embodiments, R₄ is C₆-C₁₀ aryl or 5- to 10-membered heteroaryl, wherein the aryl or heteroaryl is optionally substituted with one or more R₅ₐ₁.

In some embodiments, R₄ is C₆-C₁₀ aryl, or 5- to 10-membered heteroaryl.

In some embodiments, R₄ is C₆-C₁₀ aryl optionally substituted with one or more R₅ₐ₁.

In some embodiments, R₄ is C₆-C₁₀ aryl.

In some embodiments, R₄ is C₆ aryl (e.g., phenyl) optionally substituted with one or more R₅ₐ₁. In some embodiments, R₄ is C₆ aryl (e.g., phenyl).

In some embodiments, R₄ is C₅ aryl optionally substituted with one or more R₅ₐ₁. In some embodiments, R₄ is C₅ aryl.

In some embodiments, R₄ is C₁₀ aryl optionally substituted with one or more R₅ₐ₁. In some embodiments, R₄ is C₁₀ aryl.

In some embodiments, R₄ is 5- to 10-membered heteroaryl optionally substituted with one or more R₅ₐ₁. In some embodiments, R₄ is 5- to 10-membered heteroaryl.

In some embodiments, R₄ is 5-membered heteroaryl optionally substituted with one or more R₅ₐ₁. In some embodiments, R₄ is 5-membered heteroaryl.

In some embodiments, R₄ is 6-membered heteroaryl optionally substituted with one or more R₅ₐ₁. In some embodiments, R₄ is 6-membered heteroaryl.

In some embodiments, R₄ is 7-membered heteroaryl optionally substituted with one or more R₅ₐ₁. In some embodiments, R₄ is 7-membered heteroaryl.

In some embodiments, R₄ is 8-membered heteroaryl optionally substituted with one or more R₅ₐ₁. In some embodiments, R₄ is 8-membered heteroaryl.

In some embodiments, R₄ is 9-membered heteroaryl optionally substituted with one or more R₅ₐ₁. In some embodiments, R₄ is 9-membered heteroaryl.

In some embodiments, R₄ is 10-membered heteroaryl optionally substituted with one or more R₅ₐ₁. In some embodiments, R₄ is 10-membered heteroaryl.

In some embodiments, R₅ is H, —O—C₁-C₆ alkyl, —NH—C₁-C₆ alkyl, —NH-(5- to 10-membered heteroaryl), —NH—(C₆-C₁₀ aryl), —NH—C(O)R₅ₐ, C₁-C₆ alkyl, C₂-C₆ alkenyl, C₂-C₆ alkynyl, C₁-C₆ haloalkyl, C₆-C₁₀ aryl, or 5- to 10-membered heteroaryl, wherein the alkyl, alkenyl, alkynyl, aryl, or heteroaryl is optionally substituted with one or more R₅ₐ₁.

In some embodiments, R₅ is H.

In some embodiments, R₅ is —O—C₁-C₆ alkyl, —NH—C₁-C₆ alkyl, —NH-(5- to 10-membered heteroaryl), —NH—(C₆-C₁₀ aryl), —NH—C(O)R₅ₐ, C₁-C₆ alkyl, C₂-C₆ alkenyl, C₂-C₆ alkynyl, C₁-C₆ haloalkyl, C₆-C₁₀ aryl, or 5- to 10-membered heteroaryl, wherein the alkyl, alkenyl, alkynyl, aryl, or heteroaryl is optionally substituted with one or more R₅ₐ₁.

In some embodiments, R₅ is —O—C₁-C₆alkyl, —NH—C₁-C₆alkyl, —NH-(5- to 10-membered heteroaryl), —NH—(C₆-C₁₀ aryl), —NH—C(O)R₅ₐ, C₁-C₆ alkyl, C₂-C₆ alkenyl, C₂-C₆ alkynyl, C₁-C₆ haloalkyl, C₆-C₁₀ aryl, or 5- to 10-membered heteroaryl.

In some embodiments, R₅ is —O—C₁-C₆ alkyl, —NH—C₁-C₆ alkyl, —NH-(5- to 10-membered heteroaryl), —NH—(C₆-C₁₀ aryl), —NH—C(O)R₅ₐ, wherein the alkyl, aryl, or heteroaryl is optionally substituted with one or more R₅ₐ₁.

In some embodiments, R₅ is —O—C₁-C₆ alkyl, —NH—C₁-C₆ alkyl, —NH-(5- to 10-membered heteroaryl), —NH—(C₆-C₁₀ aryl), —NH—C(O)R₅ₐ.

In some embodiments, R₅ is —O—C₁-C₆ alkyl, wherein the alkyl is optionally substituted with one or more R₅ₐ₁. In some embodiments, R₅ is —O—C₁-C₆ alkyl.

In some embodiments, R₅ is —O—C₁ alkyl, wherein the alkyl is optionally substituted with one or more R₅ₐ. In some embodiments, R₅ is —O—C₁ alkyl.

In some embodiments, R₅ is —O—C₂ alkyl, wherein the alkyl is optionally substituted with one or more R₅ₐ₁. In some embodiments, R₅ is —O—C₂alkyl.

In some embodiments, R₅ is —O—C₃ alkyl, wherein the alkyl is optionally substituted with one or more R₅ₐ₁. In some embodiments, R₅ is —O—C₃ alkyl.

In some embodiments, R₅ is —O—C₄ alkyl, wherein the alkyl is optionally substituted with one or more R₅ₐ₁. In some embodiments, R₅ is —O—C₄ alkyl.

In some embodiments, R₅ is —O—C₈ alkyl, wherein the alkyl is optionally substituted with one or more R₅ₐ₁. In some embodiments, R₅ is —O—C₈ alkyl.

In some embodiments, R₅ is —O—C₆ alkyl, wherein the alkyl is optionally substituted with one or more R₅ₐ₁. In some embodiments, R₅ is —O—C₆ alkyl.

In some embodiments, R₅ is —NH—C₁-C₆alkyl, —NH-(5- to 10-membered heteroaryl), —NH—(C₆-C₁₀ aryl), —NH—C(O)R₅ₐ, wherein the alkyl, aryl, or heteroaryl is optionally substituted with one or more R₅ₐ₁.

In some embodiments, R₅ is —NH—C₁-C₆alkyl, —NH-(5- to 10-membered heteroaryl), —NH—(C₁-C₆ aryl), —NH—C(O)R₅ₐ.

In some embodiments, R₅ is —NH—C₁-C₆ alkyl, wherein the alkyl is optionally substituted with one or more R₅ₐ₁. In some embodiments, R₅ is —NH—C₁-C₆ alkyl.

In some embodiments, R₅ is —NH—C₁ alkyl, wherein the alkyl is optionally substituted with one or more R₅ₐ₁. In some embodiments, R₅ is —NH—C₁ alkyl.

In some embodiments, R₅ is —NH—C₂ alkyl, wherein the alkyl is optionally substituted with one or more R₅ₐ₁. In some embodiments, R₅ is —NH—C₂alkyl.

In some embodiments, R₅ is —NH—C₃ alkyl, wherein the alkyl is optionally substituted with one or more R₅ₐ₁. In some embodiments, R₅ is —NH—C₃ alkyl.

In some embodiments, R₅ is —NH—C₄ alkyl, wherein the alkyl is optionally substituted with one or more R₅ₐ₁. In some embodiments, R₅ is —NH—C₄ alkyl.

In some embodiments, R₅ is —NH—C₈ alkyl, wherein the alkyl is optionally substituted with one or more R₅ₐ₁. In some embodiments, R₅ is —NH—C₅ alkyl.

In some embodiments, R₅ is —NH—C₆ alkyl, wherein the alkyl is optionally substituted with one or more R₅ₐ₁. In some embodiments, R₅ is —NH—C₆ alkyl.

In some embodiments, R₅ is —NH-(5- to 10-membered heteroaryl), wherein the heteroaryl is optionally substituted with one or more R₅ₐ₁.

In some embodiments, R₅ is —NH-(5- to 10-membered heteroaryl).

In some embodiments, R₅ is —NH-(10-membered heteroaryl), wherein the heteroaryl is optionally substituted with one or more R₅ₐ₁. In some embodiments, R₅ is —NH-(10-membered heteroaryl).

In some embodiments, R₅ is —NH-(9-membered heteroaryl), wherein the heteroaryl is optionally substituted with one or more R₅ₐ₁. In some embodiments, R₅ is —NH-(9-membered heteroaryl).

In some embodiments, R₅ is —NH-(8-membered heteroaryl), wherein the heteroaryl is optionally substituted with one or more R₅ₐ₁. In some embodiments, R₅ is —NH-(8-membered heteroaryl).

In some embodiments, R₅ is —NH-(7-membered heteroaryl), wherein the heteroaryl is optionally substituted with one or more R₅ₐ₁. In some embodiments, R₅ is —NH-(7-membered heteroaryl).

In some embodiments, R₅ is —NH-(6-membered heteroaryl), wherein the heteroaryl is optionally substituted with one or more R₅ₐ₁. In some embodiments, R₅ is —NH-(6-membered heteroaryl).

In some embodiments, R₅ is —NH-(5-membered heteroaryl), wherein the heteroaryl is optionally substituted with one or more R₅ₐ₁. In some embodiments, R₅ is —NH-(5-membered heteroaryl).

In some embodiments, R₅ is —NH—(C₆-C₁₀ aryl), wherein the aryl is optionally substituted with one or more R₅ₐ₁. In some embodiments, R₅ is —NH—(C₆-C₁₀ aryl).

In some embodiments, R₅ is —NH—(C₆ aryl), wherein the aryl is optionally substituted with one or more R₅ₐ₁. In some embodiments, R₅ is —NH—(C₆ aryl).

In some embodiments, R₅ is —NH—(C₅ aryl), wherein the aryl is optionally substituted with one or more R₅ₐ₁. In some embodiments, R₅ is —NH—(C₅ aryl).

In some embodiments, R₅ is —NH—(C₁₀ aryl), wherein the aryl is optionally substituted with one or more R₅ₐ₁. In some embodiments, R₅ is —NH—(C₁₀ aryl).

In some embodiments, R₅ is —NH—C(O)R₅ₐ.

In some embodiments, R₅ is C₁-C₆ alkyl, C₂-C₆ alkenyl, C₂-C₆ alkynyl, or C₁-C₆ haloalkyl, wherein the alkyl, alkenyl, or alkynyl is optionally substituted with one or more R₅ₐ₁.

In some embodiments, R₅ is C₁-C₆ alkyl, C₂-C₆ alkenyl, C₂-C₆ alkynyl, or C₁-C₆ haloalkyl.

In some embodiments, R₅ is C₁-C₆ alkyl, C₂-C₆ alkenyl, or C₂-C₆ alkynyl, wherein the alkyl, alkenyl, or alkynyl is optionally substituted with one or more R₅ₐ₁.

In some embodiments, R₅ is C₁-C₆ alkyl, C₂-C₆ alkenyl, or C₂-C₆ alkynyl.

In some embodiments, R₅ is C₁-C₆ alkyl optionally substituted with one or more R₅ₐ₁. In some embodiments, R₅ is methyl optionally substituted with one or more R₅ₐ₁. In some embodiments, R₅ is ethyl optionally substituted with one or more R₅ₐ₁. In some embodiments, R₅ is propyl optionally substituted with one or more R₅ₐ₁. In some embodiments, R₅ is butyl optionally substituted with one or more R₅ₐ₁. In some embodiments, R₅ is pentyl optionally substituted with one or more R₅ₐ₁. In some embodiments, R₅ is hexyl optionally substituted with one or more R₅ₐ₁.

In some embodiments, R₅ is isopropyl optionally substituted with one or more R₅ₐ₁. In some embodiments, R₅ is isobutyl optionally substituted with one or more R₅ₐ₁. In some embodiments, R₅ is isopentyl optionally substituted with one or more R₅ₐ₁. In some embodiments, R₅ is isohexyl optionally substituted with one or more R₅ₐ₁. In some embodiments, R₅ is secbutyl optionally substituted with one or more R₅ₐ₁. In some embodiments, R₅ is secpentyl optionally substituted with one or more R₅ₐ₁. In some embodiments, R₅ is sechexyl optionally substituted with one or more R₅ₐ₁. In some embodiments, R₅ is tertbutyl optionally substituted with one or more R₅ₐ₁.

In some embodiments, R₅ is C₁-C₆ alkyl. In some embodiments, R₅ is methyl. In some embodiments, R₅ is ethyl. In some embodiments, R₅ is propyl. In some embodiments, R₅ is butyl. In some embodiments, R₅ is pentyl. In some embodiments, R₅ is hexyl. In some embodiments, R₅ is isopropyl. In some embodiments, R₅ is isobutyl. In some embodiments, R₅ is isopentyl. In some embodiments, R₅ is isohexyl. In some embodiments, R₅ is secbutyl. In some embodiments, R₅ is secpentyl. In some embodiments, R₅ is sechexyl. In some embodiments, R₅ is tertbutyl.

In some embodiments, R₅ is C₂-C₆ alkenyl optionally substituted with one or more R₅ₐ₁. In some embodiments, R₅ is C₂ alkenyl optionally substituted with one or more R₅ₐ₁. In some embodiments, R₅ is C₃ alkenyl optionally substituted with one or more R₅ₐ₁. In some embodiments, RA is C₄ alkenyl optionally substituted with one or more R₅ₐ₁. In some embodiments, R₅ is C₅ alkenyl optionally substituted with one or more R₅ₐ₁. In some embodiments, R₅ is C₆ alkenyl optionally substituted with one or more R₅ₐ₁.

In some embodiments, R₅ is C₂-C₆ alkenyl. In some embodiments, R₅ is C₂ alkenyl. In some embodiments, R₅ is C₃ alkenyl. In some embodiments, R₅ is C₄ alkenyl. In some embodiments, R₅ is C₅ alkenyl. In some embodiments, R₅ is C₆ alkenyl.

In some embodiments, R₅ is C₂-C₆ alkynyl optionally substituted with one or more R₅ₐ₁.

In some embodiments, R₅ is C₂ alkynyl optionally substituted with one or more R₅ₐ₁. In some embodiments, R₅ is C₃ alkynyl optionally substituted with one or more R₅ₐ₁. In some embodiments, R₅ is C₄ alkynyl optionally substituted with one or more R₅ₐ₁. In some embodiments, R₅ is C₅ alkynyl optionally substituted with one or more R₅ₐ₁. In some embodiments, R₅ is C₆ alkynyl optionally substituted with one or more R₅ₐ₁.

In some embodiments, R₅ is C₂-C₆ alkynyl. In some embodiments, R₅ is C₂ alkynyl. In some embodiments, R₅ is C₃ alkynyl. In some embodiments, R₅ is C₄ alkynyl. In some embodiments, R₅ is C₅ alkynyl. In some embodiments, R₅ is C₆ alkynyl.

In some embodiments, R₅ is C₁-C₆ haloalkyl, wherein the alkyl is optionally substituted with one or more R₅ₐ₁. In some embodiments, R₅ is halomethyl, wherein the alkyl is optionally substituted with one or more R₅ₐ₁. In some embodiments, R₅ is haloethyl, wherein the alkyl is optionally substituted with one or more R₅ₐ₁. In some embodiments, R₅ is halopropyl, wherein the alkyl is optionally substituted with one or more R₅ₐ₁. In some embodiments, R₅ is halobutyl, wherein the alkyl is optionally substituted with one or more R₅ₐ₁. In some embodiments, R₅ is halopentyl, wherein the alkyl is optionally substituted with one or more R₅ₐ₁. In some embodiments, R₅ is halohexyl, wherein the alkyl is optionally substituted with one or more R₅ₐ₁.

In some embodiments, R₅ is C₁-C₆ haloalkyl. In some embodiments, R₅ is halomethyl. In some embodiments, R₅ is haloethyl. In some embodiments, R₅ is halopropyl. In some embodiments, R₅ is halobutyl. In some embodiments, R₅ is halopentyl. In some embodiments, R₅ is halohexyl.

In some embodiments, R₅ is C₆-C₁₀ aryl or 5- to 10-membered heteroaryl, wherein the aryl or heteroaryl is optionally substituted with one or more R₅ₐ₁.

In some embodiments, R₅ is C₆-C₁₀ aryl, or 5- to 10-membered heteroaryl.

In some embodiments, R₅ is C₆-C₁₀ aryl optionally substituted with one or more R₅ₐ₁.

In some embodiments, 15 is C₆-C₁₀ aryl.

In some embodiments, R₅ is C₆ aryl (e.g., phenyl) optionally substituted with one or more R₅ₐ₁. In some embodiments, R₅ is C₆ aryl (e.g., phenyl).

In some embodiments, R₅ is C₅ aryl optionally substituted with one or more R₅ₐ₁. In some embodiments, R₅ is C₈ aryl.

In some embodiments, R₅ is C₁₀ aryl optionally substituted with one or more R₅ₐ₁. In some embodiments, R₅ is C₁₀ aryl.

In some embodiments, R₅ is 5- to 10-membered heteroaryl optionally substituted with one or more R₅ₐ₁. In some embodiments, R₅ is 5- to 10-membered heteroaryl.

In some embodiments, R₅ is 5-membered heteroaryl optionally substituted with one or more R₅ₐ₁. In some embodiments, R₅ is 5-membered heteroaryl.

In some embodiments, R₅ is 6-membered heteroaryl optionally substituted with one or more R₅ₐ₁. In some embodiments, R₅ is 6-membered heteroaryl.

In some embodiments, R₅ is 7-membered heteroaryl optionally substituted with one or more R₅ₐ₁. In some embodiments, R₅ is 7-membered heteroaryl.

In some embodiments, R₅ is 8-membered heteroaryl optionally substituted with one or more R₅ₐ₁. In some embodiments, R₅ is 8-membered heteroaryl.

In some embodiments, R₅ is 9-membered heteroaryl optionally substituted with one or more R₅ₐ₁. In some embodiments, R₅ is 9-membered heteroaryl.

In some embodiments, R₅ is 10-membered heteroaryl optionally substituted with one or more R₅ₐ₁. In some embodiments, R₅ is 10-membered heteroaryl.

In some embodiments, each R₄ or R₅ is independently H, —O—C₁-C₆ alkyl, —NH—C(O)R₅ₐ, C₁-C₆ alkyl, C₆-C₁₀ aryl, or 5- to 10-membered heteroaryl, wherein the alkyl, aryl, or heteroaryl is optionally substituted with one or more R₅ₐ₁.

In some embodiments, R₄ is H, —O—C₁-C₆ alkyl, —NH—C(O)R₅ₐ, C₁-C₆ alkyl, C₆-C₁₀ aryl, or 5- to 10-membered heteroaryl, wherein the alkyl, aryl, or heteroaryl is optionally substituted with one or more R₅ₐ₁.

In some embodiments, R₅ is H, —O—C₁-C₆ alkyl, —NH—C(O)R₅ₐ, C₁-C₆ alkyl, C₆-C₁₀ aryl, or 5- to 10-membered heteroaryl, wherein the alkyl, aryl, or heteroaryl is optionally substituted with one or more R₅ₐ₁.

In some embodiments, R₄ and R₅ come together to form a C₄-C₇ cycloalkyl or 4- to 10-membered heterocyclyl.

In some embodiments, R₄ and R₅ come together to form a C₄-C₇ cycloalkyl.

In some embodiments, R₄ and R₅ come together to form a C₄ cycloalkyl. In some embodiments, R₄ and R₅ come together to form a C₅ cycloalkyl. In some embodiments, R₄ and R₅ come together to form a C₆ cycloalkyl. In some embodiments, R₄ and R₅ come together to form a C₇ cycloalkyl.

In some embodiments, R₄ and R₅ come together to form a 4- to 10-membered heterocyclyl.

In some embodiments, R₄ and R₅ come together to form a 4-membered heterocyclyl. In some embodiments, R₄ and R₅ come together to form a 5-membered heterocyclyl. In some embodiments, R₄ and R₅ come together to form a 6-membered heterocyclyl. In some embodiments, R₄ and R₅ come together to form a 7-membered heterocyclyl. In some embodiments, R₄ and R₅ come together to form a 8-membered heterocyclyl. In some embodiments, R₄ and R₅ come together to form a 9-membered heterocyclyl. In some embodiments, R₄ and R₅ come together to form a 10-membered heterocyclyl.

In some embodiments, R₅ₐ is C₃-C₇ cycloalkyl, 3- to 10-membered heterocyclyl, C₆-C₁₀ aryl, or 5- to 10-membered heteroaryl, wherein the cycloalkyl, heterocyclyl, aryl, or heteroaryl is optionally substituted with one or more R₅ₐ₁.

In some embodiments, R₅ₐ is C₃-C₇ cycloalkyl, 3- to 10-membered heterocyclyl, C₆-C₁₀ aryl, or 5- to 10-membered heteroaryl.

In some embodiments, R₅ₐ is C₃-C₇ cycloalkyl or 3- to 10-membered heterocyclyl, wherein the cycloalkyl or heterocyclyl is optionally substituted with one or more R₅ₐ₁.

In some embodiments, R₅ₐ is C₃-C₇ cycloalkyl or 3- to 10-membered heterocyclyl.

In some embodiments, R₅ₐ is C₃-C₇ cycloalkyl optionally substituted with one or more R₅ₐ₁. In some embodiments, R₅ₐ is C₃-C₇ cycloalkyl.

In some embodiments, R₅ₐ is C₃ cycloalkyl optionally substituted with one or more R₅ₐ₁.

In some embodiments, R₅ₐ is C₃ cycloalkyl.

In some embodiments, R₅ₐ is C₄ cycloalkyl optionally substituted with one or more R₅ₐ₁.

In some embodiments, R₅ₐ is C₄ cycloalkyl.

In some embodiments, R₅ₐ is C₅ cycloalkyl optionally substituted with one or more R₅ₐ₁.

In some embodiments, R₅ₐ is C₅ cycloalkyl.

In some embodiments, R₅ₐ is C₆ cycloalkyl optionally substituted with one or more R₅ₐ.

In some embodiments, R₅ₐ is C₆ cycloalkyl.

In some embodiments, R₅ₐ is C₇ cycloalkyl optionally substituted with one or more R₅ₐ.

In some embodiments, R₅ₐ is C₇ cycloalkyl.

In some embodiments, R₅ₐ is 3- to 10-membered heterocyclyl optionally substituted with one or more R₅ₐ₁. In some embodiments, R₅ₐ is 3- to 10-membered heterocyclyl.

In some embodiments, R₅ₐ is 3-membered heterocyclyl optionally substituted with one or more R₅ₐ₁. In some embodiments, R₅ₐ is 3-membered heterocyclyl.

In some embodiments, R₅ₐ is 4-membered heterocyclyl optionally substituted with one or more R₅ₐ₁. In some embodiments, R₅ₐ is 4-membered heterocyclyl.

In some embodiments, R₅ₐ is 5-membered heterocyclyl optionally substituted with one or more R₅ₐ₁. In some embodiments, R₅ₐ is 5-membered heterocyclyl.

In some embodiments, R₅ₐ is 6-membered heterocyclyl optionally substituted with one or more R₅ₐ₁. In some embodiments, R₅ₐ is 6-membered heterocyclyl.

In some embodiments, R₅ₐ is 7-membered heterocyclyl optionally substituted with one or more R₅ₐ₁. In some embodiments, R₅ₐ is 7-membered heterocyclyl.

In some embodiments, R₅ₐ is 8-membered heterocyclyl optionally substituted with one or more R₅ₐ₁. In some embodiments, R₅ₐ is 8-membered heterocyclyl.

In some embodiments, R₅ₐ is 9-membered heterocyclyl optionally substituted with one or more R₅ₐ₁. In some embodiments, R₅ₐ is 9-membered heterocyclyl.

In some embodiments, R₅ₐ is 10-membered heterocyclyl optionally substituted with one or more R₅ₐ₁. In some embodiments, R₅ₐ is 10-membered heterocyclyl.

In some embodiments, R₅ₐ is C₆-C₁₀ aryl or 5- to 10-membered heteroaryl, wherein the aryl or heteroaryl is optionally substituted with one or more R₅ₐ₁.

In some embodiments, R₅ₐ is C₆-C₁₀ aryl or 5- to 10-membered heteroaryl.

In some embodiments, R₅ₐ is C₆-C₁₀ aryl optionally substituted with one or more R₅ₐ₁.

In some embodiments, R₅ₐ is C₆-C₁₀ aryl.

In some embodiments, R₅ₐ is C₆ aryl (e.g., phenyl) optionally substituted with one or more R₅ₐ₁. In some embodiments, R₅ₐ is C₆ aryl (e.g., phenyl).

In some embodiments, R₅ₐ is C₅ aryl optionally substituted with one or more R₅ₐ₁. In some embodiments, R₅ₐ is C₆ aryl.

In some embodiments, R₅ₐ is C₁₀ aryl optionally substituted with one or more R₅ₐ₁. In some embodiments, R₅ₐ is C₁₀ aryl.

In some embodiments, R₅ₐ is 5- to 10-membered heteroaryl optionally substituted with one or more R₅ₐ₁. In some embodiments, R₅ₐ is 5- to 10-membered heteroaryl.

In some embodiments, R₅ₐ is 5-membered heteroaryl optionally substituted with one or more R₅ₐ₁. In some embodiments, R₅ₐ is 5-membered heteroaryl.

In some embodiments, R₅ₐ is 6-membered heteroaryl optionally substituted with one or more R₅ₐ₁. In some embodiments, R₅ₐ is 6-membered heteroaryl.

In some embodiments, R₅ₐ is 7-membered heteroaryl optionally substituted with one or more R₅ₐ₁. In some embodiments, R₅ₐ is 7-membered heteroaryl.

In some embodiments, R₅ₐ is 8-membered heteroaryl optionally substituted with one or more R₅ₐ₁. In some embodiments, R₅ₐ is 8-membered heteroaryl.

In some embodiments, R₅ₐ is 9-membered heteroaryl optionally substituted with one or more R₅ₐ₁. In some embodiments, R₅, is 9-membered heteroaryl.

In some embodiments, R₅ₐ is 10-membered heteroaryl optionally substituted with one or more R₅ₐ₁. In some embodiments, R₅, is 10-membered heteroaryl.

In some embodiments, R₅ₐ is halogen, —CN, —(CH₂)ₚ—N(R_{5a1′})(R_{5b1′}), —O—C₁-C₆ alkyl, —(CH₂)ₚ—C₃-C₇ cycloalkyl wherein the cycloalkyl is optionally substituted with one or more —C(O)OH, —OH or —NH₂, C₁-C₆ alkyl optionally substituted with one or more halogen, —COOH, —OH, or —NH₂, or 3- to 10-membered heterocyclyl optionally substituted with one or more —(C₁-C₆ alkyl)-OH.

In some embodiments, R₅ₐ₁ is halogen, —CN, —(CH₂)ₚ—N(R_{5a1′})(R_{5b1′}), —O—C₁-C₆alkyl, C₃-C₇ cycloalkyl, C₁-C₆, alkyl optionally substituted with one or more —OH or —NH₂, or 3- to 10-membered heterocyclyl optionally substituted with one or more —(C₁-C₆ alkyl)-OH.

In some embodiments, R₅ₐ₁ is halogen, —CN, —(CH₂)ₚ—N(R_{5a1′})(R_{5b1′}), —O—C₁-C₆ alkyl, —(CH₂)ₚ—C₃-C₇ cycloalkyl wherein the cycloalkyl is optionally substituted with one or more —C(O)OH, —OH or —NH₂, C₁-C₆ alkyl optionally substituted with one or more halogen, —COOH, —OH, or —NH₂.

In some embodiments, R₅ₐ₁ is halogen, —CN, —(CH₂)ₚ—N(R_{5a1′})(R_{5b1′}), —O—C₁-C₆alkyl, C₃-C₇ cycloalkyl, C₁-C₆ alkyl optionally substituted with one or more —OH or —NH₂.

In some embodiments, R₅ₐ₁ is halogen or —CN.

In some embodiments, R₅ₐ₁ is halogen. In some embodiments, R₅ₐ₁ is F, Cl, Br, or I. In some embodiments, R₅ₐ₁ is F. In some embodiments, R₅ₐ₁ is Cl. In some embodiments, R₅ₐ₁ is Br.

In some embodiments, R₅ₐ₁ is I.

In some embodiments, R₅ₐ₁ is —CN.

In some embodiments, R₅ₐ₁ is —(CH₂)ₚ—N(R_{5a1′})(R_{5b1′}) or —O—C₁-C₆ alkyl.

In some embodiments, R₅ₐ₁ is —(CH₂)ₚ—N(R₅ₐ₁)(R_{5b1′}). In some embodiments, R₅ₐ₁ is —N(R_{5a1′})(R_{5b1′}). In some embodiments, R₅ₐ₁ is —(CH₂)₁—N(R_{5a1′})(R_{5b1′}). In some embodiments, R₅ₐ₁ is —(CH₂)₂—N(R_{5a1′})(R_{5b1′}). In some embodiments, R₅ₐ₁ is —(CH₂)₃—N(R_{5a1′})(R_{5b1′}). In some embodiments, R₅ₐ₁ is —(CH₂)₄—N(R_{5a1′})(R_{5b1′}).

In some embodiments, R₅ₐ₁ is —O—C₁-C₆ alkyl. In some embodiments, R₅ₐ₁ is —O—C₁ alkyl. In some embodiments, R₅ₐ₁ is —O—C₂ alkyl. In some embodiments, R₅ₐ₁ is —O—C₃ alkyl. In some embodiments, R₅ₐ₁ is —O—C₄ alkyl. In some embodiments, R₅ₐ₁ is —O—C₅ alkyl. In some embodiments, R₅ₐ₁ is —O—C₆ alkyl.

In some embodiments, R₅ₐ₁ is C₃-C₇ cycloalkyl wherein the cycloalkyl is optionally substituted with one or more —C(O)OH, —OH or —NH₂. In some embodiments, R₅ₐ₁ is —(CH₂)₁—C₃-C₇ cycloalkyl wherein the cycloalkyl is optionally substituted with one or more —C(O)OH, —OH or —NH₂. In some embodiments, R₅ₐ₁ is —(CH₂)₂-C₃-C₇ cycloalkyl wherein the cycloalkyl is optionally substituted with one or more —C(O)OH, —OH or —NH₂. In some embodiments, R₅ₐ₁ is —(CH₂)₃—C₃-C₇ cycloalkyl wherein the cycloalkyl is optionally substituted with one or more —C(O)OH, —OH or —NH₂. In some embodiments, R₅ₐ₁ is —(CH₂)₄-C₃-C₇ cycloalkyl wherein the cycloalkyl is optionally substituted with one or more —C(O)OH, —OH or —NH₂.

In some embodiments, R₅ₐ₁ is —(CH₂)ₚ—C₃-C₇ cycloalkyl wherein the cycloalkyl is optionally substituted with one or more —C(O)OH, —OH or —NH₂. In some embodiments, R₅ₐ₁ is —(CH₂)ₚ—C₃ cycloalkyl wherein the cycloalkyl is optionally substituted with one or more —C(O)OH, —OH or —NH₂. In some embodiments, R₅ₐ₁ is —(CH₂)ₚ—C₄ cycloalkyl wherein the cycloalkyl is optionally substituted with one or more —C(O)OH, —OH or —NH₂. In some embodiments, R₅ₐ₁ is —(CH₂)ₚ—C₅ cycloalkyl wherein the cycloalkyl is optionally substituted with one or more —C(O)OH, —OH or —NH₂. In some embodiments, R₅ₐ₁ is —(CH₂)ₚ—C₆ cycloalkyl wherein the cycloalkyl is optionally substituted with one or more —C(O)OH, —OH or —NH₂. In some embodiments, R₅ₐ₁ is —(CH₂)ₚ—C₇ cycloalkyl wherein the cycloalkyl is optionally substituted with one or more —C(O)OH, —OH or —NH₂.

In some embodiments, R₅ₐ₁ is C₃-C₇ cycloalkyl wherein the cycloalkyl is optionally substituted with one or more —C(O)OH, —OH or —NH₂. In some embodiments, R₅ₐ₁ is C₃ cycloalkyl wherein the cycloalkyl is optionally substituted with one or more —C(O)OH, —OH or —NH₂. In some embodiments, R₅ₐ₁ is C₄ cycloalkyl wherein the cycloalkyl is optionally substituted with one or more —C(O)OH, —OH or —NH₂. In some embodiments, R₅ₐ₁ is C₅ cycloalkyl wherein the cycloalkyl is optionally substituted with one or more —C(O)OH, —OH or —NH₂. In some embodiments, R₅ₐ₁ is C₆ cycloalkyl wherein the cycloalkyl is optionally substituted with one or more —C(O)OH, —OH or —NH₂. In some embodiments, R₅ₐ₁ is C₇ cycloalkyl wherein the cycloalkyl is optionally substituted with one or more —C(O)OH, —OH or —NH₂.

In some embodiments, R₅ₐ₁ is C₃-C₇ cycloalkyl. In some embodiments, R₅ₐ₁ is —(CH₂)₁-C₃-C₇ cycloalkyl. In some embodiments, R₅ₐ₁ is —(CH₂)₂-C₃-C₇ cycloalkyl. In some embodiments, R₅ₐ₁ is —(CH₂)₃—C₃-C₇ cycloalkyl. In some embodiments, R₅ₐ₁ is —(CH₂)₄-C₃-C₇ cycloalkyl.

In some embodiments, R₅ₐ₁ is —(CH₂)ₚ—C₃-C₇ cycloalkyl. In some embodiments, R₅ₐ₁ is —(CH₂)ₚ—C₃ cycloalkyl. In some embodiments, R₅ₐ₁ is —(CH₂)ₚ—C₄ cycloalkyl. In some embodiments, R₅ₐ₁ is —(CH₂)ₚ—C₅ cycloalkyl. In some embodiments, R₅ₐ₁ is —(CH₂)ₚ—C₆ cycloalkyl. In some embodiments, R₅ₐ₁ is —(CH₂)ₚ—C₇ cycloalkyl.

In some embodiments, R₅ₐ₁ is C₃-C₇ cycloalkyl. In some embodiments, R₅ₐ₁ is C₃ cycloalkyl. In some embodiments, R₅ₐ₁ is C₄ cycloalkyl. In some embodiments, R₅ₐ₁ is C₅ cycloalkyl. In some embodiments, R₅ₐ₁ is C₆ cycloalkyl. In some embodiments, R₅ₐ₁ is C₇ cycloalkyl.

In some embodiments, R₅ₐ₁ is C₁-C₆ alkyl optionally substituted with one or more halogen, —COOH, —OH, or —NH₂. In some embodiments, R₅ₐ₁ is C₁-C₆ alkyl substituted with halogen, —COOH, —OH, or —NH₂.

In some embodiments, R₅ₐ₁ is C₁-C₆ alkyl optionally substituted with one or more —OH or —NH₂. In some embodiments, R₅ₐ₁ is C₁-C₆ alkyl substituted with —OH or —NH₂.

In some embodiments, R₅ₐ₁ is methyl optionally substituted with one or more halogen, —COOH, —OH, or —NH₂. In some embodiments, R₅ₐ₁ is ethyl optionally substituted with one or more halogen, —COOH, —OH, or —NH₂. In some embodiments, R₅ₐ₁ is propyl optionally substituted with one or more halogen, —COOH, —OH, or —NH₂. In some embodiments, R₅ₐ₁ is butyl optionally substituted with one or more halogen, —COOH, —OH, or —NH₂. In some embodiments, R₅ₐ₁ is pentyl optionally substituted with one or more halogen, —COOH, —OH, or —NH₂. In some embodiments, R₅ₐ₁ is hexyl optionally substituted with one or more halogen, —COOH, —OH, or —NH₂. In some embodiments, R₅ₐ₁ is isopropyl optionally substituted with one or more halogen, —COOH, —OH, or —NH₂. In some embodiments, R₅ₐ₁ is isobutyl optionally substituted with one or more halogen, —COOH, —OH, or —NH₂. In some embodiments, R₅ₐ₁ is isopentyl optionally substituted with one or more halogen, —COOH, —OH, or —NH₂. In some embodiments, R₅ₐ₁ is isohexyl optionally substituted with one or more halogen, —COOH, —OH, or —NH₂. In some embodiments, R₅ₐ₁ is secbutyl optionally substituted with one or more halogen, —COOH, —OH, or —NH₂. In some embodiments, R₅ₐ₁ is secpentyl optionally substituted with one or more halogen, —COOH, —OH, or —NH₂. In some embodiments, R₅ₐ₁ is sechexyl optionally substituted with one or more halogen, —COOH, —OH, or —NH₂. In some embodiments, R₅ₐ₁ is tertbutyl optionally substituted with one or more halogen, —COOH, —OH, or —NH₂.

In some embodiments, R₅ₐ₁ is methyl optionally substituted with one or more —OH or —NH₂. In some embodiments, R₅ₐ₁ is ethyl optionally substituted with one or more —OH or —NH₂. In some embodiments, R₅ₐ₁ is propyl optionally substituted with one or more —OH or —NH₂. In some embodiments, R₅ₐ₁ is butyl optionally substituted with one or more —OH or —NH₂. In some embodiments, R₅ₐ₁ is pentyl optionally substituted with one or more —OH or —NH₂. In some embodiments, R₅ₐ₁ is hexyl optionally substituted with one or more —OH or —NH₂. In some embodiments, R₅ₐ₁ is isopropyl optionally substituted with one or more —OH or —NH₂. In some embodiments, R₅ₐ₁ is isobutyl optionally substituted with one or more —OH or —NH₂. In some embodiments, R₅ₐ₁ is isopentyl optionally substituted with one or more —OH or —NH₂. In some embodiments, R₅ₐ₁ is isohexyl optionally substituted with one or more —OH or —NH₂. In some embodiments, R₅ₐ₁ is secbutyl optionally substituted with one or more —OH or —NH₂. In some embodiments, R₅ₐ₁ is secpentyl optionally substituted with one or more —OH or —NH₂. In some embodiments, R₅ₐ₁ is sechexyl optionally substituted with one or more —OH or —NH₂. In some embodiments, R₅ₐ₁ is tertbutyl optionally substituted with one or more —OH or —NH₂.

In some embodiments, R₅ₐ₁ is C₁-C₆ alkyl optionally substituted with one or more halogen.

In some embodiments, R₅ₐ₁ is C₁-C₆ alkyl substituted with halogen.

In some embodiments, R₅ₐ₁ is C₁-C₆ alkyl optionally substituted with one or more —COOH.

In some embodiments, R₅ₐ₁ is C₁-C₆ alkyl substituted with —COOH.

In some embodiments, R₅ₐ₁ is C₁-C₆ alkyl optionally substituted with one or more —OH. In some embodiments, R₅ₐ₁ is C₁-C₆ alkyl substituted with —OH.

In some embodiments, R₅ₐ₁ is C₁-C₆ alkyl optionally substituted with one or more —NH₂.

In some embodiments, R₅ₐ₁ is C₁-C₆ alkyl substituted with —NH₂.

In some embodiments, R₅ₐ₁ is C₁-C₆ alkyl. In some embodiments, R₅ₐ₁ is methyl. In some embodiments, R₅ₐ₁ is ethyl. In some embodiments, R₅ₐ₁ is propyl. In some embodiments, R₅ₐ₁ is butyl. In some embodiments, R₅ₐ₁ is pentyl. In some embodiments, R₅ₐ₁ is hexyl. In some embodiments, R₅ₐ₁ is isopropyl. In some embodiments, R₅ₐ₁ is isobutyl. In some embodiments, R₅ₐ₁ is isopentyl. In some embodiments, R₅ₐ₁ is isohexyl. In some embodiments, R₅ₐ₁ is secbutyl.

In some embodiments, R₅ₐ₁ is secpentyl. In some embodiments, R₅ₐ₁ is sechexyl. In some embodiments, R₅ₐ₁ is tertbutyl.

In some embodiments, R₅ₐ₁ is 3- to 10-membered heterocyclyl optionally substituted with one or more —(C₁-C₆ alkyl)-OH. In some embodiments, R₅ₐ₁ is 3- to 10-membered heterocyclyl.

In some embodiments, R₅ₐ₁ is 3-membered heterocyclyl optionally substituted with one or more —(C₁-C₆ alkyl)-OH. In some embodiments, R₅ₐ₁ is 3-membered heterocyclyl.

In some embodiments, R₅ₐ₁ is 4-membered heterocyclyl optionally substituted with one or more —(C₁-C₆ alkyl)-OH. In some embodiments, R₅ₐ₁ is 4-membered heterocyclyl.

In some embodiments, R₅ₐ₁ is 5-membered heterocyclyl optionally substituted with one or more —(C₁-C₆ alkyl)-OH. In some embodiments, R₅ₐ₁ is 5-membered heterocyclyl.

In some embodiments, R₅ₐ₁ is 6-membered heterocyclyl optionally substituted with one or more —(C₁-C₆ alkyl)-OH. In some embodiments, R₅ₐ₁ is 6-membered heterocyclyl.

In some embodiments, R₅ₐ₁ is 7-membered heterocyclyl optionally substituted with one or more —(C₁-C₆ alkyl)-OH. In some embodiments, R₅ₐ₁ is 7-membered heterocyclyl.

In some embodiments, R₅ₐ₁ is 8-membered heterocyclyl optionally substituted with one or more —(C₁-C₆ alkyl)-OH. In some embodiments, R₅ₐ₁ is 8-membered heterocyclyl.

In some embodiments, R₅ₐ₁ is 9-membered heterocyclyl optionally substituted with one or more —(C₁-C₆ alkyl)-OH. In some embodiments, R₅ₐ₁ is 9-membered heterocyclyl.

In some embodiments, R₅ₐ₁ is 10-membered heterocyclyl optionally substituted with one or more —(C₁-C₆ alkyl)-OH. In some embodiments, R₅ₐ₁ is 10-membered heterocyclyl.

In some embodiments, R₅ₐ₁ is —O—C₁-C₆ alkyl, C₃-C₇ cycloalkyl, C₁-C₆ alkyl optionally substituted with one or more —OH or —NH₂, or 3- to 10-membered heterocyclyl optionally substituted with one or more —(C₁-C₆ alkyl)-OH.

In some embodiments, R_{5a1′} is H or C₁-C₆ alkyl.

In some embodiments, R_{5b1′} is H.

In some embodiments, R_{5b1′} is C₁-C₆ alkyl. In some embodiments, R_{5b1′} is methyl. In some embodiments, R_{5b1′} is ethyl. In some embodiments, R_{5b1′} is propyl. In some embodiments, R_{5b1′} is butyl. In some embodiments, R_{5b1′} is pentyl. In some embodiments, R_{5b1′} is hexyl. In some embodiments, R_{5b1′} is isopropyl. In some embodiments, R_{5b1′} is isobutyl. In some embodiments, R_{5b1′} is isopentyl. In some embodiments, R_{5b1′} is isohexyl. In some embodiments, R_{5b1′} is secbutyl.

In some embodiments, R_{5b1′} is secpentyl. In some embodiments, R_{5b1′} is sechexyl. In some embodiments, R_{5b1′} is tertbutyl.

In some embodiments, R_{5a1′″} is H. C₁-C₆ alkyl, C₂-C₆ alkenyl, C₂-C₆ alkynyl, C₁-C₆ haloalkyl, C₃-C₅ cycloalkyl, 3- to 10-membered heterocyclyl, C₆-C₁₀ aryl, or 5- to 10-membered heteroaryl, wherein the alkyl, alkenyl, alkynyl, cycloalkyl, heterocyclyl, aryl, or heteroaryl is optionally substituted with one or more R_{5b1″}.

In some embodiments, R_{5a1′} is H, C₁-C₆ alkyl, C₂-C₆ alkenyl, C₂-C₆ alkynyl, C₁-C₆ haloalkyl, C₃-C₇ cycloalkyl, 3- to 10-membered heterocyclyl, C₆-C₁₀ aryl, or 5- to 10-membered heteroaryl, wherein the alkyl, alkenyl, alkynyl, cycloalkyl, heterocyclyl, aryl, or heteroaryl is optionally substituted with one or more R_{5a1″}.

In some embodiments, R_{5a1′} is H.

In some embodiments, R_{5a1′} is C₁-C₆ alkyl, C₂-C₆ alkenyl, C₂-C₆ alkynyl, C₁-C₆ haloalkyl, C₃-C₈ cycloalkyl, 3- to 10-membered heterocyclyl, C₆-C₁₀ aryl, or 5- to 10-membered heteroaryl, wherein the alkyl, alkenyl, alkynyl, cycloalkyl, heterocyclyl, aryl, or heteroaryl is optionally substituted with one or more R_{5a1″}.

In some embodiments, R_{5a1″} is C₁-C₆ alkyl, C₂-C₆ alkenyl, C₂-C₆ alkynyl, C₁-C₆ haloalkyl, C₃-C₇ cycloalkyl, 3- to 10-membered heterocyclyl, C₁-C₁₀ aryl, or 5- to 10-membered heteroaryl, wherein the alkyl, alkenyl, alkynyl, cycloalkyl, heterocyclyl, aryl, or heteroaryl is optionally substituted with one or more R_{5a1″}.

In some embodiments, R_{5a1′} is C₁-C₆ alkyl, C₂-C₆ alkenyl, C₂-C₆ alkynyl, C₁-C₆ haloalkyl, C₃-C₈ cycloalkyl, 3- to 10-membered heterocyclyl, C₆-C₁₀ aryl, or 5- to 10-membered heteroaryl.

In some embodiments, R_{5a1′} is C₁-C₆ alkyl, C₂-C₆ alkenyl, C₂-C₆ alkynyl, C₁-C₆ haloalkyl, C₃-C₇ cycloalkyl, 3- to 10-membered heterocyclyl, C₆-C₁₀ aryl, or 5- to 10-membered heteroaryl.

In some embodiments, R_{5a1′} is C₁-C₆ alkyl, C₂-C₆ alkenyl, C₂-C₆ alkynyl, or C₁-C₆ haloalkyl, wherein the alkyl, alkenyl, or alkynyl is optionally substituted with one or more R_{5a1″}.

In some embodiments, R_{5a1′} is C₁-C₆ alkyl, C₂-C₆ alkenyl, C₂-C₆ alkynyl, or C₁-C₆ haloalkyl.

In some embodiments, R_{5a1′} is C₁-C₆₆ alkyl, C₂-C₆ alkenyl, or C₂-C₆ alkynyl, wherein the alkyl, alkenyl, or alkynyl is optionally substituted with one or more R_{5a1″}.

In some embodiments, R_{5a1′} is C₁-C₆ alkyl, C₂-C₆ alkenyl, or C₂-C₆ alkynyl.

In some embodiments, R_{5a1′} is C₁-C₆ alkyl optionally substituted with one or more R_{5a1″}.

In some embodiments, R₅ₐ₁ is methyl optionally substituted with one or more R_{5a1″}. In some embodiments, R_{5a1′} is ethyl optionally substituted with one or more R_{5a1″}. In some embodiments, R₅ₐ₁ is propyl optionally substituted with one or more R_{5a1′}. In some embodiments, R_{5a1′} is butyl optionally substituted with one or more R_{5a1″}. In some embodiments, R_{5a1″} is pentyl optionally substituted with one or more R₅ₐ₁. In some embodiments, R₅ₐ₁ is hexyl optionally substituted with one or more R_{5a1′}. In some embodiments, R_{5a1′} is isopropyl optionally substituted with one or more R_{5a1″}. In some embodiments, R_{5a1′} is isobutyl optionally substituted with one or more R_{5a1″}. In some embodiments, R_{5a1′} is isopentyl optionally substituted with one or more R_{5a1′}. In some embodiments, R_{5a1′} is isohexyl optionally substituted with one or more R_{5a1″}. In some embodiments, R_{5a1′} is secbutyl optionally substituted with one or more R_{5a1″}. In some embodiments, R_{5a1′} is secpentyl optionally substituted with one or more R_{5a1″}. In some embodiments, R_{5a1′} is sechexyl optionally substituted with one or more R_{5a1″}. In some embodiments, R_{5a1′} is tertbutyl optionally substituted with one or more R_{5a1′}.

In some embodiments, R_{5a1′} is C₁-C₆ alkyl. In some embodiments, R_{5a1″}, is methyl. In some embodiments, R_{5a1′} is ethyl. In some embodiments, R₅ₐ₁, is propyl. In some embodiments, R_{5a1′} is butyl. In some embodiments, R_{5a1′} is pentyl. In some embodiments, R_{5a1″} is hexyl. In some embodiments, R₅ₐ₁ is isopropyl. In some embodiments, R_{5a1″} is isobutyl. In some embodiments, R₅ₐ₁ is isopentyl. In some embodiments, R_{5a1′} is isohexyl. In some embodiments, R_{5a1′} is secbutyl.

In some embodiments, R_{5a1′} is secpentyl. In some embodiments, R_{5a1′} is sechexyl. In some embodiments, R_{5a1′} is tertbutyl.

In some embodiments, R_{5a1′} is C₂-C₆ alkenyl optionally substituted with one or more R_{5a1′}. In some embodiments, R_{5a1′} is C₂ alkenyl optionally substituted with one or more R_{5a1″}. In some embodiments, R_{5a1′} is C₃ alkenyl optionally substituted with one or more R_{5a1″}. In some embodiments, R_{5a1′} is C₄ alkenyl optionally substituted with one or more R_{5a1′}. In some embodiments, R_{5a1′} is C₅ alkenyl optionally substituted with one or more R_{5a1″}. In some embodiments, R_{5a1′} is C₆ alkenyl optionally substituted with one or more R_{5a1″}.

In some embodiments, R₅ₐ₁ is C₂-C₆ alkenyl. In some embodiments, R_{5a1″} is C₂ alkenyl. In some embodiments, R_{5a1′} is C₃ alkenyl. In some embodiments, R_{5a1″} is C₄ alkenyl. In some embodiments, R_{5a1′} is C₅ alkenyl. In some embodiments, R_{5a1′} is C₆ alkenyl.

In some embodiments, R_{5a1′} is C₂-C₆ alkynyl optionally substituted with one or more R_{5a1′}. In some embodiments, R_{5a1′} is C₂ alkynyl optionally substituted with one or more R₅ₐ₁. In some embodiments, R_{5a1′} is C₃ alkynyl optionally substituted with one or more R_{5a1′}. In some embodiments, R₅ₐ₁ is C₄ alkynyl optionally substituted with one or more R₅ₐ₁. In some embodiments, R_{5a1′} is C₅ alkynyl optionally substituted with one or more R_{5a1′}. In some embodiments, R₅ₐ₁ is C₆ alkynyl optionally substituted with one or more R_{5a1′}.

In some embodiments, R₅ₐ₁ is C₂-C₆ alkynyl. In some embodiments, R_{5a1″} is C₂ alkynyl. In some embodiments, R_{5a1″} is C₃ alkynyl. In some embodiments, R_{5a1′} is C₄ alkynyl. In some embodiments, R_{5a1′} is C₅ alkynyl. In some embodiments, R_{5a1″} is C₆ alkynyl.

In some embodiments, R_{5a1′} is C₁-C₆ haloalkyl optionally substituted with one or more R_{5a1′}. In some embodiments, R_{5a1′″} is halomethyl optionally substituted with one or more R_{5a1″}. In some embodiments, R_{5a1′} is haloethyl optionally substituted with one or more R_{5a1″}. In some embodiments, R_{5a1′} is halopropyl optionally substituted with one or more R_{5a1″}. In some embodiments, R₅ₐ₁, is halobutyl optionally substituted with one or more R_{5a1′}. In some embodiments, R_{5a1′} is halopentyl optionally substituted with one or more R_{5a1′}. In some embodiments, R_{5a1′} is halohexyl optionally substituted with one or more R_{5a1″}.

In some embodiments, R_{5a1′″} is C₁-C₆ haloalkyl. In some embodiments, R₅ₐ₁, is halomethyl.

In some embodiments, R₅ₐ₁, is haloethyl. In some embodiments, R₅ₐ₁ is halopropyl. In some embodiments, R₅ₐ₁, is halobutyl. In some embodiments, R_{5a1′} is halopentyl. In some embodiments, R_{5a1′} is halohexyl.

In some embodiments, R_{5a1′} is C₃-C₈ cycloalkyl, 3- to 10-membered heterocyclyl, C₆-C₁₀ aryl, or 5- to 10-membered heteroaryl, wherein the cycloalkyl, heterocyclyl, aryl, or heteroaryl is optionally substituted with one or more R-ar.

In some embodiments, R_{5a1′} is C₃-C₇ cycloalkyl, 3- to 10-membered heterocyclyl, C₆-C₁₀ aryl, or 5- to 10-membered heteroaryl, wherein the cycloalkyl, heterocyclyl, aryl, or heteroaryl is optionally substituted with one or more R_{5a1″}.

In some embodiments, R_{5a1′} is C₃-C₈ cycloalkyl, 3- to 10-membered heterocyclyl, C₆-C₁₀ aryl, or 5- to 10-membered heteroaryl.

In some embodiments, R_{5a1′} is C₃-C₇ cycloalkyl, 3- to 10-membered heterocyclyl, C₆-C₁₀ aryl, or 5- to 10-membered heteroaryl.

In some embodiments, R_{5a1′} is C₃-C₈ cycloalkyl or 3- to 10-membered heterocyclyl, wherein the cycloalkyl or heterocyclyl is optionally substituted with one or more R₅ₐ₁.

In some embodiments, R_{5a1′} is C₃-C₇ cycloalkyl or 3- to 10-membered heterocyclyl, wherein the cycloalkyl or heterocyclyl is optionally substituted with one or more R₅ₐ₁.

In some embodiments, R_{5a1′} is C₃-C₈ cycloalkyl or 3- to 10-membered heterocyclyl.

In some embodiments, R₅ₐ₁ is C₃-C₇ cycloalkyl or 3- to 10-membered heterocyclyl.

In some embodiments, R_{5a1′} is C₃-C₇ cycloalkyl optionally substituted with one or more R_{5a1″}.

In some embodiments, R_{5a1′} is C₃-C₈ cycloalkyl. In some embodiments, R₅ₐ₁, is C₃-C₇ cycloalkyl.

In some embodiments, R₅ₐ₁ is C₃ cycloalkyl optionally substituted with one or more R_{5a1″}. In some embodiments, R_{5a1′} is C₃ cycloalkyl.

In some embodiments, R₅ₐ₁ is C₄ cycloalkyl optionally substituted with one or more R_{5a1″}. In some embodiments, R_{5a1′} is C₄ cycloalkyl.

In some embodiments, R₅ₐ₁ is C₅ cycloalkyl optionally substituted with one or more R_{5a1″}. In some embodiments, R_{5a1′} is C₅ cycloalkyl.

In some embodiments, R_{5a1′} is C₆ cycloalkyl optionally substituted with one or more R_{5a1″}. In some embodiments, R_{5a1′} is C₃-C₇ cycloalkyl.

In some embodiments, R_{5a1′} is C₇ cycloalkyl optionally substituted with one or more R_{5a1″}. In some embodiments, R_{5a1′} is C₇ cycloalkyl.

In some embodiments, R_{5a1′} is C₈ cycloalkyl optionally substituted with one or more R_{5a1″} in some embodiments, R_{5a1′} is C₈ cycloalkyl.

In some embodiments, R_{5a1′} is 3- to 10-membered heterocyclyl optionally substituted with one or more R_{5a1′}. In some embodiments, R₅ₐ₁ is 3- to 10-membered heterocyclyl.

In some embodiments, R_{5a1′} is 3-membered heterocyclyl optionally substituted with one or more R_{5a1″}. In some embodiments, R₅ₐ₁, is 3-membered heterocyclyl.

In some embodiments, R_{5a1′} is 4-membered heterocyclyl optionally substituted with one or more R_{5a1″}. In some embodiments, R₅ₐ₁, is 4-membered heterocyclyl.

In some embodiments, R_{5a1′} is 5-membered heterocyclyl optionally substituted with one or more R_{5a1″}. In some embodiments, R_{5a1′} is 5-membered heterocyclyl.

In some embodiments, R₅ₐ₁ is 6-membered heterocyclyl optionally substituted with one or more R_{5a1″}. In some embodiments, R_{5a1′} is 6-membered heterocyclyl.

In some embodiments, R₅ₐ₁ is 7-membered heterocyclyl optionally substituted with one or more R_{5a1″}. In some embodiments, R_{5a1′} is 7-membered heterocyclyl.

In some embodiments, R₅ₐ₁ is 8-membered heterocyclyl optionally substituted with one or more R_{5a1″}. In some embodiments, R_{5a1′} is 8-membered heterocyclyl.

In some embodiments, R_{5a1′} is 9-membered heterocyclyl optionally substituted with one or more R_{5a1″}. In some embodiments, R_{5a1′} is 9-membered heterocyclyl.

In some embodiments, R_{5a1′} is 10-membered heterocyclyl optionally substituted with one or more R_{5a1″}. In some embodiments, R₅ₐ₁ is 10-membered heterocyclyl.

In some embodiments, R_{5a1′} is C₁-C₁₀ aryl or 5- to 10-membered heteroaryl, wherein the aryl or heteroaryl is optionally substituted with one or more R_{5a1″}.

In some embodiments, R_{5a1′} is C₆-C₁₀ aryl or 5- to 10-membered heteroaryl.

In some embodiments, R_{5a1′} is C₆-C₁₀ aryl optionally substituted with one or more R_{5a1′″}.

In some embodiments, R_{5a1′} is C₆-C₁₀ aryl.

In some embodiments, R_{5a1″} is C₆ aryl (e.g., phenyl) optionally substituted with one or more R_{5a1″}. In some embodiments, R_{5a1′} is C₆ aryl (e.g., phenyl).

In some embodiments, R₅ₐ₁, is C₈ aryl optionally substituted with one or more R_{5a1″}. In some embodiments, R_{5a1′} is C₈ aryl.

In some embodiments, R_{5a1′} is C₁₀ aryl optionally substituted with one or more R_{5a1″}. In some embodiments, R_{5a1′} is C₁₀ aryl.

In some embodiments, R_{5a1′} is 5- to 10-membered heteroaryl optionally substituted with one or more R_{5a1″}. In some embodiments, R₅ₐ₁ is 5- to 10-membered heteroaryl.

In some embodiments, R_{5a1′} is 5-membered heteroaryl optionally substituted with one or more R_{5a1″}. In some embodiments, R_{5a1′} is 5-membered heteroaryl.

In some embodiments, R_{5a1″} is 6-membered heteroaryl optionally substituted with one or more R_{5a1″}. In some embodiments, R_{5a1′} is 6-membered heteroaryl.

In some embodiments, R_{5a1″} is 7-membered heteroaryl optionally substituted with one or more R_{5a1″}. In some embodiments, R_{5a1′} is 7-membered heteroaryl.

In some embodiments, R₅ₐ₁ is 8-membered heteroaryl optionally substituted with one or more R_{5a1″}. In some embodiments, R_{5a1′} is 8-membered heteroaryl.

In some embodiments, R_{5a1′} is 9-membered heteroaryl optionally substituted with one or more R_{5a1″}. In some embodiments, R_{5a1′} is 9-membered heteroaryl.

In some embodiments, R_{5a1′} is 10-membered heteroaryl optionally substituted with one or more R_{5a1″}. In some embodiments, R_{5a1′} is 10-membered heteroaryl.

In some embodiments, R_{5a1′} is H, C₁-C₆ alkyl, C₁-C₆ haloalkyl, C₃-C₇ cycloalkyl, 3- to 10-membered heterocyclyl, C₆-C₁₀ aryl, or 5- to 10-membered heteroaryl, wherein the alkyl, cycloalkyl, heterocyclyl, aryl, or heteroaryl is optionally substituted with one or more R_{5a1″}.

In some embodiments, R_{5a1′} and R_{5a1″} come together to form a 3- to 10-membered heterocyclyl optionally substituted with one or more R_{5a1′″}. In some embodiments, R_{5a1′} and R_{5a1″} come together to form a 3- to 10-membered heterocyclyl substituted with one or more R_{5a1″}. In some embodiments, R_{5a1′} and R_{5a1″} come together to form a 3- to 10-membered heterocyclyl substituted with one R_{5a1″}. In some embodiments, R_{5a1′} and R_{5a1″} come together to form a 3- to 10-membered heterocyclyl substituted with two R_{5a1″}.

In some embodiments, R_{5a1′} and R br come together to form a 3-membered heterocyclyl optionally substituted with one or more R_{5a1′}. In some embodiments, R_{5a1′} and R_{5a1″} come together to form a 3-membered heterocyclyl substituted with one or more R_{5a1″}. In some embodiments, R_{5a1′} and R_{5a1″} come together to form a 3-membered heterocyclyl substituted with one R_{5a1″}. In some embodiments, R_{5a1′} and R_{5a1″} come together to form a 3-membered heterocyclyl substituted with two R_{5a1″}.

In some embodiments, R_{5a1″} and R_{5a1″} come together to form a 4-membered heterocyclyl optionally substituted with one or more R_{5a1′}. In some embodiments, R_{5a1′} and R_{5b1′} come together to form a 4-membered heterocyclyl substituted with one or more R₅ₐ. In some embodiments, R_{5a1′} and R_{5a1″} come together to form a 4-membered heterocyclyl substituted with one R_{5a1″}. In some embodiments, R_{5a1″} and R_{5a1″} come together to form a 4-membered heterocyclyl substituted with two R_{5a1″}.

In some embodiments, R_{5a1″} and R_{5a1″} come together to form a 5-membered heterocyclyl optionally substituted with one or more R_{5a1′″}. In some embodiments, R_{5a1′} and R_{5a1″} come together to form a 5-membered heterocyclyl substituted with one or more R_{5a1″}. In some embodiments, R_{5a1″} and R_{5a1″} come together to form a 5-membered heterocyclyl substituted with one R_{5a1″}. In some embodiments, R₅ₐ₁, and R_{5a1″} come together to form a 5-membered heterocyclyl substituted with two R_{5a1″}.

In some embodiments, R₅ₐ₁, and R_{5a1″} come together to form a 6-membered heterocyclyl optionally substituted with one or more R_{5a1′}. In some embodiments, R_{5a1′} and R_{5b1′} come together to form a 6-membered heterocyclyl substituted with one or more R_{5a1′}. In some embodiments, R_{5a1′} and R_{5b1″} come together to form a 6-membered heterocyclyl substituted with one R_{5a1′}. In some embodiments, R_{5a1′} and R_{5a1″} come together to form a 6-membered heterocyclyl substituted with two R_{5a1′}.

In some embodiments, R_{5a1″} and R_{5b1″} come together to form a 7-membered heterocyclyl optionally substituted with one or more R_{5a1′}. In some embodiments, R_{5a1′} and R_{5a1″} come together to form a 7-membered heterocyclyl substituted with one or more R_{5a1′}. In some embodiments, R_{5a1′} and R_{5b1′} come together to form a 7-membered heterocyclyl substituted with one R_{5a1′}. In some embodiments, R_{5a1′} and R_{5b1″} come together to form a 7-membered heterocyclyl substituted with two R_{5a1′}.

In some embodiments, R_{5a1′} and R_{5b1′} come together to form a 8-membered heterocyclyl optionally substituted with one or more R_{5a1″}. In some embodiments, R_{5a1′} and R_{5b1′} come together to form a 8-membered heterocyclyl substituted with one or more R_{5a1′}. In some embodiments, R_{5a1′} and R_{5a1″} come together to form a 8-membered heterocyclyl substituted with one R_{5a1″}. In some embodiments, R₅ₐ₁ and R_{5a1″} come together to form a 8-membered heterocyclyl substituted with two R_{5a1′}.

In some embodiments, R₅ₐ₁ and R_{5b1} come together to form a 9-membered heterocyclyl optionally substituted with one or more R_{5a1′}. In some embodiments, R_{5a1″} and R_{5a1″} come together to form a 9-membered heterocyclyl substituted with one or more R_{5a1′}. In some embodiments, R_{5a1″} and R_{5a1″} come together to form a 9-membered heterocyclyl substituted with one R_{5a1″}. In some embodiments, R_{5a1′} and R_{5b1″} come together to form a 9-membered heterocyclyl substituted with two R_{5a1′}.

In some embodiments, R_{5a1′} and R_{5b1″} come together to form a 10-membered heterocyclyl optionally substituted with one or more R_{5a1′}. In some embodiments, R_{5a1′} and R_{5b1′} come together to form a 10-membered heterocyclyl substituted with one or more R_{5a1″}. In some embodiments, R₅ₐ₁ and R_{5a1″} come together to form a 10-membered heterocyclyl substituted with one R₅ₐ₁. In some embodiments, R₅ₐ₁ and R_{5b1′} come together to form a 10-membered heterocyclyl substituted with two R_{5a1′}.

In some embodiments, R₅ₐ₁ is oxo, —CN, —OH, C₁-C₆ alkyl optionally substituted with one or more —C(O)OH, —OH or —NH₂, C₂-C₆ alkenyl, C₂-C₆ alkynyl, C₁-C₆ haloalkyl, —C(O)OH, —C(O)C₁-C₆ alkyl, —C(O)O—C₁-C₆ alkyl, —C(O)NH₂, —C(O)NH—C₁-C₆ alkyl, —C(O)NH—C₃-C₁₀ cycloalkyl, —NH—C₁-C₆ alkyl-R_{5a1′″}, —(CH₂)_{q}—(C₃-C₇ cycloalkyl), or —(CH₂)_{q}-(3- to 10-membered heterocyclyl), wherein the cycloalkyl or heterocyclyl is optionally substituted with one or more oxo, C₁-C₆ alkyl, C₂-C₆ alkenyl, C₂-C₆ alkynyl, —C(O)OH, or —OH.

In some embodiments, R₅ₐ₁ is oxo, —CN, —OH, C₁-C₆ alkyl, C₂-C₆ alkenyl, C₂-C₆ alkynyl, C₁-C₆ haloalkyl, —C(O)OH, —C(O)C₁-C₆ alkyl, —C(O)O—C₁-C₆ alkyl, —C(O)NH—C₁-C₆ alkyl, —C(O)NH—C₃-C₁₀ cycloalkyl, —NH—C₁-C₆ alkyl-R_{5a1′″}, —(CH₂)_{q}—(C₃-C₇ cycloalkyl), or —(CH₂)_{q}-(3- to 10-membered heterocyclyl), wherein the cycloalkyl or heterocyclyl is optionally substituted with one or more oxo, C₁-C₆ alkyl, C₂-C₆ alkenyl, C₂-C₆ alkynyl, —C(O)OH, or —OH.

In some embodiments, R₅ₐ₁ is oxo, —CN, —OH, C₂-C₆ alkyl optionally substituted with one or more —C(O)OH, —OH or —NH₂, C₂-C₆ alkenyl, C₂-C₆ alkynyl, C₁-C₆ haloalkyl, —C(O)OH, —C(O)C₁-C₆ alkyl, —C(O)O—C₁-C₆ alkyl, —C(O)NH₂, —C(O)NH—C₁-C₆ alkyl, —C(O)NH—C₃-C₁₀ cycloalkyl, or —NH—C₁-C₆ alkyl-R_{5a1′″}.

In some embodiments, R_{5a1″} is oxo, —CN, —OH, C₁-C₆ alkyl, C₂-C₆ alkenyl, C₂-C₆ alkynyl, C₁-C₆ haloalkyl, —C(O)OH, —C(O)C₁-C₆ alkyl, —C(O)O—C₁-C₆ alkyl, —C(O)NH—C₁-C₆ alkyl, or —NH—C₁-C₆ alkyl-R_{5a1′}.

In some embodiments, R_{5a1″} is oxo, —CN, or —OH.

In some embodiments, R₅ₐ₁ is oxo. In some embodiments, R_{5a1′} is —CN. In some embodiments, R_{5a1′} is —OH.

In some embodiments, R_{5a1′} is C₁-C₆ alkyl optionally substituted with one or more —C(O)OH, —OH or —NH₂, C₂-C₆ alkenyl, C₂-C₆ alkynyl, or C₁-C₆ haloalkyl.

In some embodiments, R_{5a1′} is C₁-C₆ alkyl, C₂-C₆ alkenyl, C₂-C₆ alkynyl, or C₁-C₆ haloalkyl.

In some embodiments, R_{5a1′} is C₁-C₆ alkyl optionally substituted with one or more —C(O)OH, —OH or —NH₂, C₂-C₆ alkenyl, or C₂-C₆ alkynyl.

In some embodiments, R_{5a1″} is C₁-C₆ alkyl, C₂-C₆ alkenyl, or C₂-C₆ alkynyl.

In some embodiments, R_{5a1′} is C₁-C₆ alkyl optionally substituted with one or more —C(O)OH, —OH or —NH₂. In some embodiments, R_{5a1′} is methyl optionally substituted with one or more —C(O)OH, —OH or —NH₂. In some embodiments, R_{5a1′} is ethyl optionally substituted with one or more —C(O)OH, —OH or —NH₂. In some embodiments, R₅ₐ₁ is propyl optionally substituted with one or more —C(O)OH, —OH or —NH₂. In some embodiments, R_{5a1′} is butyl optionally substituted with one or more —C(O)OH, —OH or —NH₂. In some embodiments, R_{5a1′} is pentyl optionally substituted with one or more —C(O)OH, —OH or —NH₂. In some embodiments, R_{5a1″} is hexyl optionally substituted with one or more —C(O)OH, —OH or —NH₂. In some embodiments, R_{5a1″} is isopropyl optionally substituted with one or more —C(O)OH, —OH or —NH₂. In some embodiments, R_{5a1″} is isobutyl optionally substituted with one or more —C(O)OH, —OH or —NH₂.

In some embodiments, R_{5a1″} is isopentyl optionally substituted with one or more —C(O)OH, —OH or —NH₂. In some embodiments, R_{5a1′} is isohexyl optionally substituted with one or more —C(O)OH, —OH or —NH₂. In some embodiments, R_{5a1″} is secbutyl optionally substituted with one or more —C(O)OH, —OH or —NH₂. In some embodiments, R_{5a1′} is secpentyl optionally substituted with one or more —C(O)OH, —OH or —NH₂. In some embodiments, R_{5a1″} is sechexyl optionally substituted with one or more —C(O)OH, —OH or —NH₂. In some embodiments, R_{5a1′} is tertbutyl optionally substituted with one or more —C(O)OH, —OH or —NH₂.

In some embodiments, R_{5a1′} is C₁-C₆ alkyl optionally substituted with one or more —C(O)OH. In some embodiments, R_{5a1″} is C₁-C₆ alkyl substituted with —C(O)OH.

In some embodiments, R_{5a1′} is C₁-C₆ alkyl optionally substituted with one or more —OH.

In some embodiments, R_{5a1′} is C₁-C₆ alkyl substituted with —OH.

In some embodiments, R_{5a1″} is C₁-C₆ alkyl optionally substituted with one or more —NH₂.

In some embodiments, R_{5a1′} is C₁-C₆ alkyl substituted with —NH₂.

In some embodiments, R_{5a1′} is C₁-C₆ alkyl. In some embodiments, R_{5a1′} is methyl. In some embodiments, R_{5a1′} is ethyl. In some embodiments, R_{5a1′} is propyl. In some embodiments, R_{5a1″} is butyl. In some embodiments, R_{5a1′} is pentyl. In some embodiments, R_{5a1′} is hexyl. In some embodiments, R_{5a1″} is isopropyl. In some embodiments, R₅ₐ₁ is isobutyl. In some embodiments, R_{5a1′} is isopentyl. In some embodiments, R_{5a1′} is isohexyl. In some embodiments, R_{5a1′} is secbutyl. In some embodiments, R_{5a1′} is secpentyl. In some embodiments, R_{5a1′} is sechexyl. In some embodiments, R_{5a1′} is tertbutyl.

In some embodiments, R_{5a1′} is C₂-C₆ alkenyl. In some embodiments, R_{5a1″} is C₂ alkenyl.

In some embodiments, R_{5a1″} is C₃ alkenyl. In some embodiments, R₅ₐ₁ is C₄ alkenyl. In some embodiments, R_{5a1″} is C₅ alkenyl. In some embodiments, R_{5a1″} is C₆ alkenyl.

In some embodiments, R_{5a1″} is C₂-C₆ alkynyl. In some embodiments, R_{5a1″} is C₂ alkynyl. In some embodiments, R_{5a1″} is C₃ alkynyl. In some embodiments, R_{5a1″} is C₄ alkynyl. In some embodiments, R_{5a1′} is C₅ alkynyl. In some embodiments, R₅ₐ₁ is C₆ alkynyl.

In some embodiments, R_{5a1′} is C₁-C₆ haloalkyl. In some embodiments, R_{5a1″} is halomethyl. In some embodiments, R_{5a1″} is haloethyl. In some embodiments, R_{5a1″} is halopropyl. In some embodiments, R_{5a1″} is halobutyl. In some embodiments, R_{5a1″} is halopentyl. In some embodiments, R_{5a1″} is halohexyl.

In some embodiments, R_{5a1″} is —C(O)OH, —C(O)C₁-C₆ alkyl, —C(O)O—C₁-C₆ alkyl, —C(O)NH₂, —C(O)NH—C₁-C₆ alkyl, or —NH—C₁-C₆ alkyl-R_{5a1′″}.

In some embodiments, R_{5a1′} is —C(O)OH, —C(O)C₁-C₆ alkyl, —C(O)O—C₁-C₆ alkyl, —C(O)NH—C₁-C₆ alkyl, or —NH—C₁-C₆ alkyl-R_{5a1″}.

In some embodiments, R_{5a1″} is —C(O)OH, —C(O)C₁-C₆ alkyl, or —C(O)O—C₁-C₆ alkyl.

In some embodiments, R_{5a1″} is —C(O)OH.

In some embodiments, R_{5a1″} is —C(O)C₁-C₆ alkyl.

In some embodiments, R_{5a1′} is —C(O)C₁ alkyl. In some embodiments, R_{5a1′} is —C(O)C₂ alkyl. In some embodiments, R_{5a1′} is —C(O)C₃ alkyl. In some embodiments, R_{5a1″} is —C(O)C₄ alkyl. In some embodiments, R_{5a1′} is —C(O)C₅ alkyl. In some embodiments, R_{5a1′} is —C(O)C₆ alkyl.

In some embodiments, R_{5a1″} is —C(O)O—C₁-C₆ alkyl.

In some embodiments, R_{5a1′} is —C(O)O—C₁ alkyl. In some embodiments, R_{5a1″} is —C(O)O—C₂ alkyl. In some embodiments, R_{5a1′} is —C(O)O—C₃ alkyl. In some embodiments, R₅ₐ₁ is —C(O)O—C₄ alkyl. In some embodiments, R₅ₐ₁ is —C(O)O—C₈ alkyl. In some embodiments, R_{5a1′} is —C(O)O—C₆ alkyl.

In some embodiments, R_{5a1″} is —C(O)NH₂, —C(O)NH—C₁-C₆ alkyl, or —NH—C₁-C₆ alkyl-R_{5a1″}.

In some embodiments, R_{5a1″} is —C(O)NH—C₁-C₆ alkyl or —NH—C₁-C₆ alkyl-R_{5a1″}.

In some embodiments, R_{5a1″} is —C(O)NH₂.

In some embodiments, R_{5a1″} is —C(O)NH—C₁-C₆ alkyl.

In some embodiments, R_{5a1″} is —C(O)NH—C₁ alkyl. In some embodiments, R₅ₐ₁ is —C(O)NH—C₂ alkyl. In some embodiments, R_{5a1″} is —C(O)NH—C₃ alkyl. In some embodiments, R_{5a1″} is —C(O)NH—C₄ alkyl. In some embodiments, R_{5a1″} is —C(O)NH—C₈ alkyl. In some embodiments, R₅ₐ₁ is —C(O)NH—C₆ alkyl.

In some embodiments, R_{5a1′} is —NH—C₁-C₆ alkyl-R_{5a1″}.

In some embodiments, R_{5a1′} is —NH—C₁ alkyl-R_{5a1′″}. In some embodiments, R_{5a1″} is —NH—C₂ alkyl-R_{5a1″}. In some embodiments, R_{5a1′} is —NH—C₃ alkyl-R₅ₐ₁. In some embodiments, Rai is —NH—C₄ alkyl-R_{5a1′″}. In some embodiments, R_{5a1′} is —NH—C₅ alkyl-R_{5a1′}. In some embodiments, R_{5a1′} is —NH—C₆ alkyl-R_{5a1′}.

In some embodiments, R_{5a1″} is —C(O)NH—C₃-C₁₀ cycloalkyl, —(CH₂)_{q}—(C₃-C₇ cycloalkyl), or —(CH₂)_{q}-(3- to 10-membered heterocyclyl), wherein the cycloalkyl or heterocyclyl is optionally substituted with one or more oxo, C₁-C₆ alkyl, C₂-C₆ alkenyl, C₂-C₆ alkynyl, —C(O)OH, or —OH.

In some embodiments, R_{5a1′} is —C(O)NH—C₃-C₁₀ cycloalkyl, —(CH₂)_{q}—(C₃-C₇ cycloalkyl), or —(CH_{Z})_{q}-(3- to 10-membered heterocyclyl), wherein the cycloalkyl and heterocyclyl are substituted with one or more oxo, C₁-C₆ alkyl, C₂-C₆ alkenyl, C₂-C₆ alkynyl, —C(O)OH, or —OH.

In some embodiments, R_{5a1″} is —C(O)NH—C₇-C₁₀ cycloalkyl, —(CH₂)_{q}—(C₃-C₇ cycloalkyl), or —(CH₂)_{q}-(3- to 10-membered heterocyclyl), wherein the cycloalkyl and heterocyclyl are substituted with one oxo, C₁-C₆ alkyl, C₂-C₆ alkenyl, C₂-C₆ alkynyl, —C(O)OH, or —OH.

In some embodiments, R_{5a1″} is —C(O)NH—C₃-C₁₀ cycloalkyl, —(CH₂)_{q}—(C₃-C₇ cycloalkyl), or —(CH₂)_{q}-(3- to 10-membered heterocyclyl), wherein the cycloalkyl and heterocyclyl are substituted with two oxo, C₁-C₆ alkyl, C₂-C₆ alkenyl, C₂-C₆ alkynyl, —C(O)OH, or —OH.

In some embodiments, R_{5a1′} is —C(O)NH—C₃-C₁₀ cycloalkyl, wherein the cycloalkyl is optionally substituted with one or more oxo, C₁-C₆ alkyl, C₂-C₆ alkenyl, C₂-C₆ alkynyl, —C(O)OH, or —OH.

In some embodiments, R_{5a1′} is —C(O)NH—C₃-C₁₀ cycloalkyl, wherein the cycloalkyl is substituted with one or more oxo, C₁-C₆ alkyl, C₂-C₆ alkenyl, C₂-C₆ alkynyl, —C(O)OH, or —OH.

In some embodiments, R_{5a1″} is —C(O)NH—C₃-C₁₀ cycloalkyl, wherein the cycloalkyl is substituted with one oxo, C₁-C₆ alkyl, C₂-C₆ alkenyl, C₂-C₆ alkynyl, —C(O)OH, or —OH.

In some embodiments, R₅ₐ₁ is —C(O)NH—C₃-C₁₀ cycloalkyl, wherein the cycloalkyl is substituted with two oxo, C₁-C₆ alkyl, C₂-C₆ alkenyl, C₂-C₆ alkynyl, —C(O)OH, or —OH.

In some embodiments, R_{5a1′} is —(CH₂)_{q}—(C₃-C₇ cycloalkyl), wherein the cycloalkyl is optionally substituted with one or more oxo, C₁-C₆ alkyl, C₂-C₆ alkenyl, C₂-C₆ alkynyl, —C(O)OH, or —OH.

In some embodiments, R_{5a1′} is (CH₂)_{q}—(C₃-C₇ cycloalkyl), wherein the cycloalkyl is substituted with one or more oxo, C₁-C₆ alkyl, C₂-C₆ alkenyl, C₂-C₆ alkynyl, —C(O)OH, or —OH.

In some embodiments, R_{5a1″} is —(CH₂)_{q}—(C₃-C₇ cycloalkyl), wherein the cycloalkyl is substituted with one oxo, C₁-C₆ alkyl, C₂-C₆ alkenyl, C₂-C₆ alkynyl, —C(O)OH, or —OH.

In some embodiments, R_{5a1″} is —(CH₂)_{q}—(C₃-C₇ cycloalkyl), wherein the cycloalkyl is substituted with two oxo, C₁-C₆ alkyl, C₂-C₆ alkenyl, C₂-C₆ alkynyl, —C(O)OH, or —OH.

In some embodiments, R₅ₐ₁ is —(CH₂)_{q}-(3- to 10-membered heterocyclyl), wherein the heterocyclyl is optionally substituted with one or more oxo, C₁-C₆ alkyl, C₂-C₆ alkenyl, C₂-C₆ alkynyl, —C(O)OH, or —OH.

In some embodiments, R_{5a1′} is —(CH₂)_{q}-(3- to 10-membered heterocyclyl), wherein the heterocyclyl is substituted with one or more oxo, C₁-C₆ alkyl, C₂-C₆ alkenyl, C₂-C₆ alkynyl, —C(O)OH, or —OH.

In some embodiments, R₅ₐ₁ is —(CH₂)_{q}-(3- to 10-membered heterocyclyl), wherein the heterocyclyl is substituted with one oxo, C₁-C₆ alkyl, C₂-C₆ alkenyl, C₂-C₆, alkynyl, —C(O)OH, or —OH.

In some embodiments, R_{5a1′} is —(CH₂)_{q}-(3- to 10-membered heterocyclyl), wherein the heterocyclyl is substituted with two oxo, C₁-C₆ alkyl, C₂-C₆ alkenyl, C₂-C₆ alkynyl, —C(O)OH, or —OH.

In some embodiments, R_{5a1″} is oxo, —OH, C₁-C₆ alkyl, —C(O)OH, —C(O)C₁-C₆ alkyl, —C(O)O—C₁-C₆ alkyl, —C(O)NH—C₁-C₆ alkyl, —C(O)NH—C₃-C₁₀ cycloalkyl, —NH—C₁-C₆ alkyl-R_{5a1″}, —(CH₂)_{q}—(C₃-C₇ cycloalkyl), or —(CH₂)_{q}-(3- to 10-membered heterocyclyl), wherein the cycloalkyl or heterocyclyl is optionally substituted with one or more oxo, C₁-C₆ alkyl, —C(O)OH, or —OH.

In some embodiments, R_{5a1′″} is 3- to 10-membered heterocyclyl optionally substituted with oxo. In some embodiments, R_{5a1′″} is 3- to 10-membered heterocyclyl substituted with oxo.

In some embodiments, R_{5a1′″} is 3-membered heterocyclyl optionally substituted with oxo.

In some embodiments, R_{5a1′} is 3-membered heterocyclyl substituted with oxo.

In some embodiments, R_{5a1′″} is 4-membered heterocyclyl optionally substituted with oxo.

In some embodiments, R_{5a1′″} is 4-membered heterocyclyl substituted with oxo.

In some embodiments, R_{5a1′″} is 5-membered heterocyclyl optionally substituted with oxo.

In some embodiments, R_{5a1′″} is 5-membered heterocyclyl substituted with oxo.

In some embodiments, R_{5a1′″} is 6-membered heterocyclyl optionally substituted with oxo.

In some embodiments, R_{5a1′″} is 6-membered heterocyclyl substituted with oxo.

In some embodiments, R_{5a1′″} is 7-membered heterocyclyl optionally substituted with oxo.

In some embodiments, R_{5a1′″} is 7-membered heterocyclyl substituted with oxo.

In some embodiments, R_{5a1′″} is 8-membered heterocyclyl optionally substituted with oxo.

In some embodiments, R_{5a1′″} is 8-membered heterocyclyl substituted with oxo.

In some embodiments, R_{5a1′″} is 9-membered heterocyclyl optionally substituted with oxo.

In some embodiments, R_{5a1′″} is 9-membered heterocyclyl substituted with oxo.

In some embodiments, R_{5a1′″} is 10-membered heterocyclyl optionally substituted with oxo.

In some embodiments, R_{5a1′″} is 10-membered heterocyclyl substituted with oxo.

In some embodiments, each R₆ is independently H, halogen, —CN, C₁-C₆ alkyl, C₂-C₆ alkenyl, C₂-C₆ alkynyl, C₁-C₆ haloalkyl, —O—C₁-C₆alkyl, —NH—C₁-C₆alkyl, or C₃-C₇ cycloalkyl.

In some embodiments, each R₆ is independently H.

In some embodiments, each R₆ is independently halogen, —CN, C₁-C₆ alkyl, C₂-C₆ alkenyl, C₂-C₆ alkynyl, C₁-C₆ haloalkyl, —O—C₁-C₆alkyl, —NH—C₁-C₆ alkyl, or C₃-C₇ cycloalkyl.

In some embodiments, each R₆ is independently halogen or —CN.

In some embodiments, each R₆ is independently halogen.

In some embodiments, each R₆ is independently F, Cl, Br, or I.

In some embodiments, each R₆ is independently F. In some embodiments, each R₆ is independently C₁. In some embodiments, each R₆ is independently Br. In some embodiments, each R₆ is independently 1.

In some embodiments, each R₆ is independently —CN.

In some embodiments, each R₆ is independently C₁-C₆ alkyl, C₂-C₆ alkenyl, C₂-C₆ alkynyl, C₁-C₆ haloalkyl, —O—C₁-C₆ alkyl, —NH—C₁-C₆ alkyl, or C₃-C₇ cycloalkyl.

In some embodiments, each R₆ is independently C₁-C₆ alkyl, C₂-C₆ alkenyl, C₂-C₆ alkynyl, or C₁-C₆ haloalkyl.

In some embodiments, R₆ is C₁-C₆ alkyl, C₂-C₆ alkenyl, or C₂-C₆ alkynyl.

In some embodiments, R₆ is C₁-C₆ alkyl.

In some embodiments, R₆ is methyl. In some embodiments, R₆ is ethyl. In some embodiments, R₆ is propyl. In some embodiments, R₆ is butyl. In some embodiments, R₆ is pentyl. In some embodiments, R₆ is hexyl. In some embodiments, R is isopropyl. In some embodiments, R₆ is isobutyl. In some embodiments, R₆ is isopentyl. In some embodiments, R₆ is isohexyl. In some embodiments, R₆ is secbutyl. In some embodiments, R₆ is secpentyl. In some embodiments, R₆ is sechexyl. In some embodiments, R₆ is tertbutyl.

In some embodiments, R₆ is C₂-C₆ alkenyl. In some embodiments, R₆ is C₂ alkenyl. In some embodiments, R₆ is C₃ alkenyl. In some embodiments, R₆ is C₄ alkenyl. In some embodiments, R₆ is C₅ alkenyl. In some embodiments, R₆ is C₆ alkenyl.

In some embodiments, R₆ is C₂-C₆ alkynyl. In some embodiments, R₆ is C₂ alkynyl. In some embodiments, R₆ is C₃ alkynyl. In some embodiments, R₆ is C₄ alkynyl. In some embodiments, R₆ is C₅ alkynyl. In some embodiments, R₆ is C, alkynyl.

In some embodiments, R₆ is C₁-C₆ haloalkyl. In some embodiments, R₆ is halomethyl. In some embodiments, R₆ is haloethyl. In some embodiments, R₆ is halopropyl. In some embodiments, R₆ is halobutyl. In some embodiments, R₆ is halopentyl. In some embodiments, R₆ is halohexyl.

In some embodiments, each R₆ is independently —O—C₁-C₆ alkyl or —NH—C₁-C₆ alkyl.

In some embodiments, each R₆ is independently —O—C₁-C₆alkyl.

In some embodiments, each R₆ is independently —O—C₁ alkyl. In some embodiments, each R₆ is independently —O—C₂ alkyl. In some embodiments, each R₆ is independently —O—C₃ alkyl. In some embodiments, each R₆ is independently —O—C₄ alkyl. In some embodiments, each R₆ is independently —O—C₈ alkyl. In some embodiments, each R₆ is independently —O—C₆ alkyl.

In some embodiments, each R₆ is independently —NH—C₁-C₆ alkyl.

In some embodiments, each R₆ is independently —NH—C₁ alkyl. In some embodiments, each R₆ is independently —NH—C₂ alkyl. In some embodiments, each R₆ is independently —NH—C₃ alkyl.

In some embodiments, each R₆ is independently —NH—C₄ alkyl. In some embodiments, each R₆ is independently —NH—C₈ alkyl. In some embodiments, each R₆ is independently —NH—C₆ alkyl.

In some embodiments, each R₆ is independently C₃-C₇ cycloalkyl.

In some embodiments, each R₆ is independently C₃ cycloalkyl. In some embodiments, each R₆ is independently C₄ cycloalkyl. In some embodiments, each R₆ is independently C₅ cycloalkyl. In some embodiments, each R₆ is independently C₆ cycloalkyl. In some embodiments, each R₆ is independently C₆ cycloalkyl.

In some embodiments, each R₆ is independently H, halogen, C₁-C₆ alkyl, —O—C₁-C₆alkyl, —NH—C₁-C₆ alkyl, or C₃-C₇ cycloalkyl.

In some embodiments, R₇ is C₁-C₆ alkyl, C₂-C₆ alkenyl, C₂-C₆ alkynyl, or C₁-C₆ haloalkyl.

In some embodiments, R₇ is C₁-C₆ alkyl, C₂-C₆ alkenyl, or C₂-C₆ alkynyl.

In some embodiments, R₇ is C₁-C₆ alkyl.

In some embodiments, R₇ is methyl. In some embodiments, R₇ is ethyl. In some embodiments, R₇ is propyl. In some embodiments, R₇ is butyl. In some embodiments, R₇ is pentyl. In some embodiments, R₇ is hexyl. In some embodiments, R₇ is isopropyl. In some embodiments, R₇ is isobutyl. In some embodiments, R₇ is isopentyl. In some embodiments, R₇ is isohexyl. In some embodiments, R₇ is secbutyl. In some embodiments, R₇ is secpentyl. In some embodiments, R₇ is sechexyl. In some embodiments, R₇ is tertbutyl.

In some embodiments, R₇ is C₂-C₆ alkenyl. In some embodiments, R₇ is C₂ alkenyl. In some embodiments, R₇ is C₃ alkenyl. In some embodiments, R₇ is C₄ alkenyl. In some embodiments, R₇ is C₅ alkenyl. In some embodiments, R₇ is C₆ alkenyl.

In some embodiments, R₇ is C₂-C₆ alkynyl. In some embodiments, R₇ is C₂ alkynyl. In some embodiments, R₇ is C₃ alkynyl. In some embodiments, R₇ is C₄ alkynyl. In some embodiments, R₇ is C₅ alkynyl. In some embodiments, R₇ is C₆ alkynyl.

In some embodiments, R₇ is C₁-C₆ haloalkyl. In some embodiments, R₇ is halomethyl. In some embodiments, R₇ is haloethyl. In some embodiments, R₇ is halopropyl. In some embodiments, R₇ is halobutyl. In some embodiments, R₇ is halopentyl. In some embodiments, R₇ is halohexyl.

In some embodiments, m is 0, 1, 2, 3, or 4. In some embodiments, m is 0 or 1.

In some embodiments, m is 0. In some embodiments, m is 1. In some embodiments, m is 2. In some embodiments, m is 3. In some embodiments, m is 4.

In some embodiments, n is 0, 1, 2, 3, or 4.

In some embodiments, n is 0. In some embodiments, n is 1. In some embodiments, n is 2.

In some embodiments, n is 3. In some embodiments, n is 4.

In some embodiments, p is 0, 1, 2, 3, or 4. In some embodiments, p is 0 or 1.

In some embodiments, p is 0. In some embodiments, p is 1. In some embodiments, p is 2.

In some embodiments, p is 3. In some embodiments, p is 4.

In some embodiments, q is 0, 1, 2, 3, or 4.

In some embodiments, q is 0. In some embodiments, q is 1. In some embodiments, q is 2.

In some embodiments, q is 3. In some embodiments, q is 4.

In some embodiments, at least one of R₄, R₅, or R₆ is not H.

In some embodiments, at least one of R₄ is not H. In some embodiments, at least one of R₅ is not H. In some embodiments, at least one of R₆ is not H.

In some embodiments, when Ring B is an optionally substituted 5-membered heteroaryl or 9-membered heterocyclyl and R_{5a1′} and R_{5b1′} come together to form a 5-membered heterocyclyl substituted with one R_{5a1″}, then R_{5a1″} is not —OH.

In some embodiments, when Ring B is an optionally substituted 5-membered heteroaryl or 9-membered heterocyclyl and R₅ₐ₁ and R_{5a1″} come together to form a 5-membered heterocyclyl substituted with one R_{5a1′} then R_{5a1″} is oxo, —CN, C₁-C₆ alkyl, C₂-C₆ alkenyl, C₂-C₆ alkynyl, C₁-C₆ haloalkyl, —C(O)OH, —C(O)C₁-C₆ alkyl, —C(O)O—C₁-C₆ alkyl, —C(O)NH—C₁-C₆ alkyl, —C(O)NH—C₃-C₁₀ cycloalkyl, —NH—C₁-C₆ alkyl-R_{5a1′}, —(CH₂)_{q}—(C₃-C₇ cycloalkyl), or —(CH₂)_{q}-(3- to 10-membered heterocyclyl), wherein the cycloalkyl or heterocyclyl is optionally substituted with one or more oxo, C₁-C₆ alkyl, C₂-C₆ alkenyl, C₂-C₆ alkynyl, —C(O)OH, or —OH.

In some embodiments, when Ring B is an optionally substituted 9-membered heterocyclyl and R_{5a1′} and R_{5a1″} come together to form a 6-membered heterocyclyl, then R₂ is not —(CH₂)₂—OH.

In some embodiments, when Ring B is an optionally substituted 9-membered heterocyclyl and R_{5a1″} and R_{5b1″} come together to form a 6-membered heterocyclyl, then R₂ is not H, —(CH₂)ₙ—N(R₂ₐ)(R_{2b}), —OH, —CH₂—OH, —(CH₂)₃—OH, —(CH₂)₄—OH, —(CH₂)ₙ—O(C₁-C₆ alkyl), C₃-C₇ cycloalkyl, 3- to 10-membered heterocyclyl, C₆-C₁₀ aryl, or 5- to 10-membered heteroaryl, wherein the cycloalkyl, heterocyclyl, aryl, or heteroaryl is optionally substituted with one or more R_{2b′}.

In some embodiments, when R₄ and R₅ come together to form a 6-membered heterocyclyl

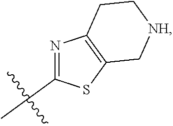 [SMILES: *c1nc2c(s1)CNCC2]

and Ring B is then Ring B is not substituted with —CH₃ or —CH₂CH₂—OH.

In some embodiments, when R₅ is —NH—C(O)R₅ₐ, R₅ₐ is not a substituted 5-membered heteroaryl.

In some embodiments, the compound is of Formula (I-a) or (1-b):

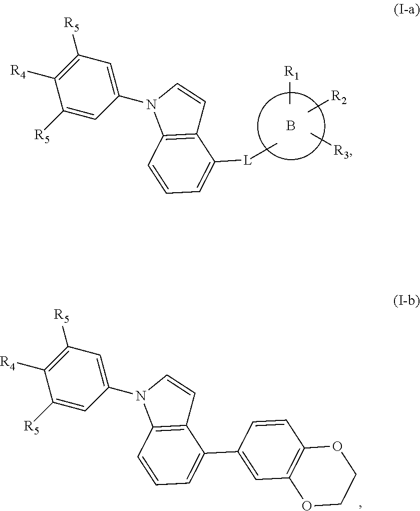

or a prodrug, solvate, or pharmaceutically acceptable salt thereof.

In some embodiments, the compound is of Formula (I-a) or a prodrug, solvate, or pharmaceutically acceptable salt thereof.

In some embodiments, the compound is of Formula (I-b) or a prodrug, solvate, or pharmaceutically acceptable salt thereof.

In some embodiments, the compound is of Formula (I-c) or (I-d):

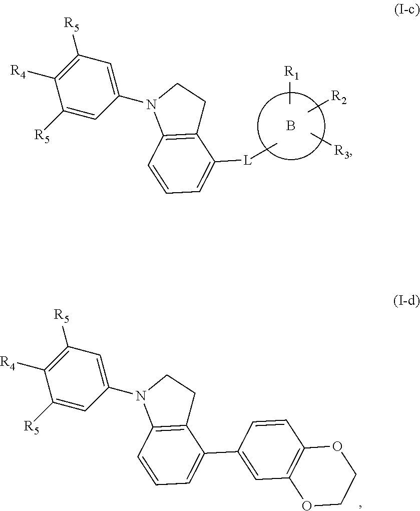

or a prodrug, solvate, or pharmaceutically acceptable salt thereof.

In some embodiments, the compound is of Formula (I-c) or a prodrug, solvate, or pharmaceutically acceptable salt thereof.

In some embodiments, the compound is of Formula (I-d) or a prodrug, solvate, or pharmaceutically acceptable salt thereof.

In some embodiments, the compound is of Formula (I-e) or (I-f):

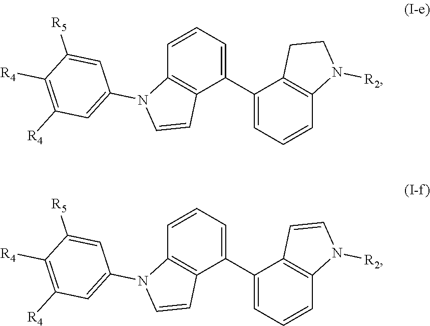

or a prodrug, solvate, or pharmaceutically acceptable salt thereof.

In some embodiments, the compound is of Formula (I-e) or a prodrug, solvate, or pharmaceutically acceptable salt thereof.

In some embodiments, the compound is of Formula (I-f) or a prodrug, solvate, or pharmaceutically acceptable salt thereof.

In some embodiments, the compound is of Formula (I-g):

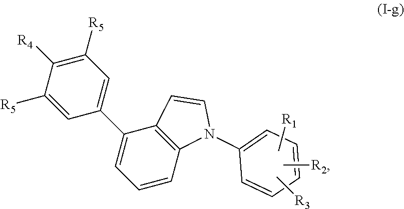

or a prodrug, solvate, or pharmaceutically acceptable salt thereof.

In some embodiments, the compound is of Formula (I-g) or a prodrug, solvate, or pharmaceutically acceptable salt thereof.

In some embodiments, the compound is of Formula (I-h) or (I-i):

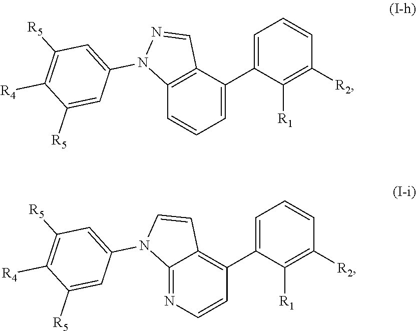

or a prodrug, solvate, or pharmaceutically acceptable salt thereof.

In some embodiments, the compound is of Formula (I-h) or a prodrug, solvate, or pharmaceutically acceptable salt thereof.

In some embodiments, the compound is of Formula (I-i) or a prodrug, solvate, or pharmaceutically acceptable salt thereof.

In some embodiments, the compound is of Formula (I-j):

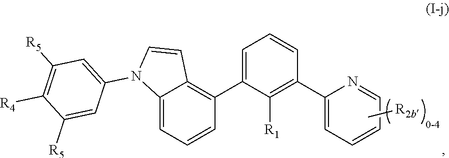

or a prodrug, solvate, or pharmaceutically acceptable salt thereof.

In some embodiments, the compound is of Formula (I-j) or a prodrug, solvate, or pharmaceutically acceptable salt thereof.

In some embodiments, the compound is of Formula (I-k):

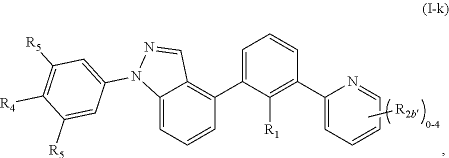

or a prodrug, solvate, or pharmaceutically acceptable salt thereof.

In some embodiments, the compound is of Formula (I-k) or a prodrug, solvate, or pharmaceutically acceptable salt thereof.

It is understood that, for a compound of any one of the formulae described herein, Ring A, Ring B, L, R₁, R₂, R₃, R₄, R₅, R₆, R₇, R₁ₐ, R₂ₐ, R_{2b}, R_{2b′}, R_{2b″}, R_{2b′″}, R₅ₐ, R₅ₐ₁, R_{5a1′}, R_{5a1″}, R_{5a1′″}, R_{5b1′}, m, n, p, or q can each be, where applicable, selected from the groups described herein, and any group described herein for any of Ring A. Ring B, L, R₁, R₂, R₃, R₄, R₅, R₆, R₇, R₁ₐ, R₂ₐ, R_{2b}, R_{2b′}, R_{2b″}, R_{2b′″}, R₅ₐ, R₅ₐ₁, R_{5a1′}, R_{5a1″}, R_{5a1′″}, R_{5b1′}, m, n, p, or q can be combined, where applicable, with any group described herein for one or more of the remainder of Ring A. Ring B, L, Rt, R₂, R₃, R₄, R₅, R₆, R₇, R₁ₐ, R₂ₐ, R₂, R_{2b′}, R_{2b″}, R_{2b′″}, R₅ₐ, R₅ₐ₁, R_{5a1′}, R_{5a1″}, R_{5a1′″}, R_{5b1′}, m, n, p, or q.

In some embodiments, the compound is selected from the compounds described in Table 1 and prodrugs and pharmaceutically acceptable salts thereof.

In some embodiments, the compound is selected from the compounds described in Table 1 and pharmaceutically acceptable salts thereof.

In some embodiments, the compound is selected from the prodrugs of compounds described in Table 1 and pharmaceutically acceptable salts thereof.

In some embodiments, the compound is selected from the compounds described in Table 1.

TABLE 1

| Compound | |
|---|---|
| No. | Structure and Name |
| 1 | 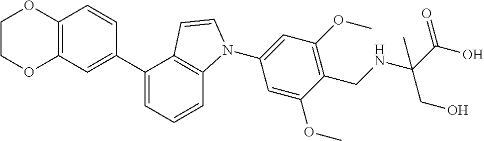 [SMILES: COc1cc(-n2ccc3c(-c4ccc5c(c4)OCCO5)cccc32)cc(OC)c1CNC(C)(CO)C(=O)O] |
| | 2-((4-(4-(2,3-dihydrobenzo[b][1,4]dioxin-6-yl)-1H-indol-1-yl)-2,6- |
| | dimethoxybenzyl)amino)-3-hydroxy-2-methylpropanoic acid |
| 2 | 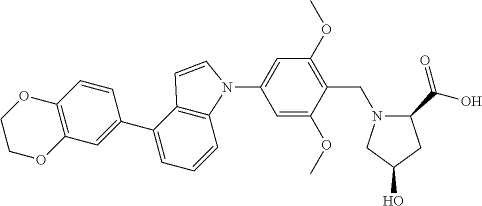 [SMILES: COc1cc(-n2ccc3c(-c4ccc5c(c4)OCCO5)cccc32)cc(OC)c1CN1C[C@H](O)C[C@@H]1C(=O)O] |
| | (2R,4R)-1-(4-(4-(2,3-dihydrobenzo[b][1,4]dioxin-6-yl)-1H-indol-1- |
| | yl)-2,6-dimethoxybenzyl)-4-hydroxypyrrolidine-2-carboxylic acid |
| 3 | 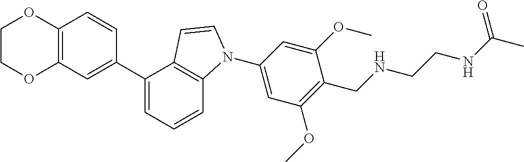 [SMILES: COc1cc(-n2ccc3c(-c4ccc5c(c4)OCCO5)cccc32)cc(OC)c1CNCCNC(C)=O] |
| | N-(2-((4-(4-(2,3-dihydrobenzo[b][1,4]dioxin-6-yl)-1H-indol-1-yl)- |
| | 2,6-dimethoxybenzyl)amino)ethyl)acetamide |
| 4 | 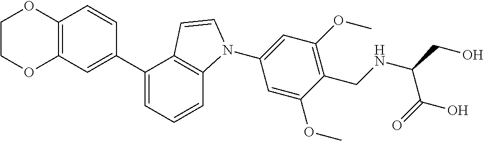 [SMILES: COc1cc(-n2ccc3c(-c4ccc5c(c4)OCCO5)cccc32)cc(OC)c1CN[C@@H](CO)C(=O)O] |
| | (4-(4-(2,3-dihydrobenzo[b][1,4]dioxin-6-yl)-1H-indol-1-yl)-2,6- |
| | dimethoxybenzyl)-L-serine |
| 5 | 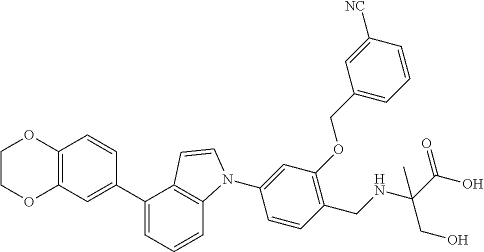 [SMILES: CC(CO)(NCc1ccc(-n2ccc3c(-c4ccc5c(c4)OCCO5)cccc32)cc1OCc1cccc(C#N)c1)C(=O)O] |
| | 2-((2-((3-cyanobenzyl)oxy)-4-(4-(2,3-dihydrobenzo[b][1,4]dioxin-6- |
| | yl)-1H-indol-1-yl)benzyl)amino)-3-hydroxy-2-methylpropanoic acid |
| 6 | 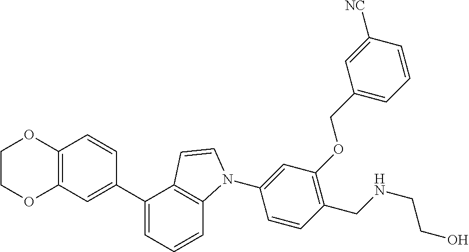 [SMILES: N#Cc1cccc(COc2cc(-n3ccc4c(-c5ccc6c(c5)OCCO6)cccc43)ccc2CNCCO)c1] |
| | 3-((5-(4-(2,3-dihydrobenzo[b][1,4]dioxin-6-yl)-1H-indol-1-yl)-2-(((2- |
| | hydroxyethyl)amino)methyl)phenoxy)methyl)benzonitrile |
| 7 | 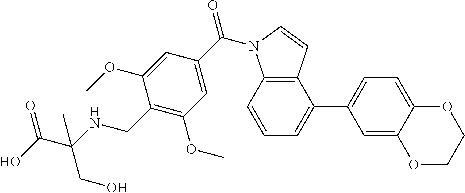 [SMILES: COc1cc(C(=O)n2ccc3c(-c4ccc5c(c4)OCCO5)cccc32)cc(OC)c1CNC(C)(CO)C(=O)O] |
| | 2-((4-(4-(2,3-dihydrobenzo[b][1,4]dioxin-6-yl)-1H-indole-1- |
| | carbonyl)-2,6-dimethoxybenzyl)amino)-3-hydroxy-2- |
| | methylpropanoic acid |
| 8 | 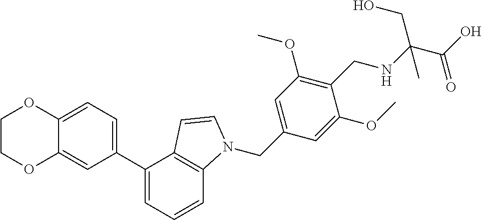 [SMILES: COc1cc(Cn2ccc3c(-c4ccc5c(c4)OCCO5)cccc32)cc(OC)c1CNC(C)(CO)C(=O)O] |
| | 2-((4-((4-(2,3-dihydrobenzo[b][1,4]dioxin-6-y])-1H-indol-1- |
| | yl)methyl)-2,6-dimethoxybenzyl)amino)-3-hydroxy-2- |
| | methylpropanoic acid |
| 9 | 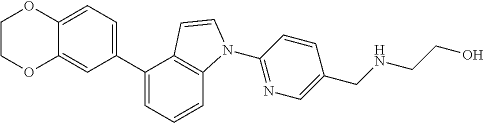 [SMILES: OCCNCc1ccc(-n2ccc3c(-c4ccc5c(c4)OCCO5)cccc32)nc1] |
| | 2-(((6-(4-(2,3-dihydrobenzo[b][1,4]dioxin-6-yl)-1H-indol-1- |
| | yl)pyridin-3-yl)methyl)amino)ethan-1-ol |
| 10 | 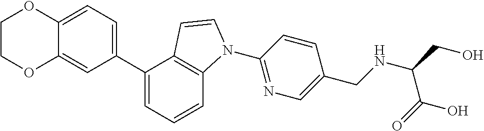 [SMILES: O=C(O)[C@H](CO)NCc1ccc(-n2ccc3c(-c4ccc5c(c4)OCCO5)cccc32)nc1] |
| | ((6-(4-(2,3-dihydrobenzo[b][1,4]dioxin-6-yl)-1H-indol-1-yl)pyridin-3- |
| | yl)methyl)-L-serine |
| 11 | 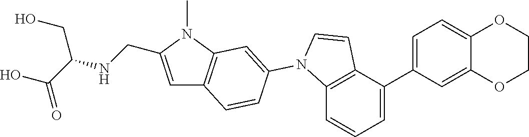 [SMILES: Cn1c(CN[C@@H](CO)C(=O)O)cc2ccc(-n3ccc4c(-c5ccc6c(c5)OCCO6)cccc43)cc21] |
| | ((4-(2,3-dihydrobenzo[b][1,4]dioxin-6-yl)-1′-methyl-l′H-[1,6′- |
| | biindol]-2′-yl)methyl)-L-serine |
| 12 | 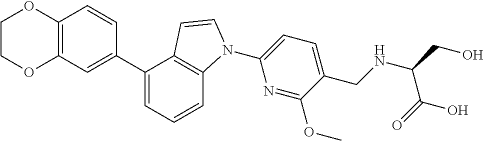 [SMILES: COc1nc(-n2ccc3c(-c4ccc5c(c4)OCCO5)cccc32)ccc1CN[C@@H](CO)C(=O)O] |
| | ((6-(4-(2,3-dihydrobenzo[b][1,4]dioxin-6-yl)-1H-indol-1-yl)~2~ |
| | methoxypyridin-3-yl)methyl)-L-serine |
| 13 | 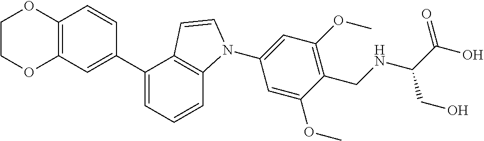 [SMILES: COc1cc(-n2ccc3c(-c4ccc5c(c4)OCCO5)cccc32)cc(OC)c1CN[C@@H](CO)C(=O)O] |
| | (4-(4-(2,3-dihydrobenzo[b][1,4]dioxin-6-yl)indolin-1-yl)-2,6- |
| | dimethoxybenzyl)-L-serine |
| 14 | 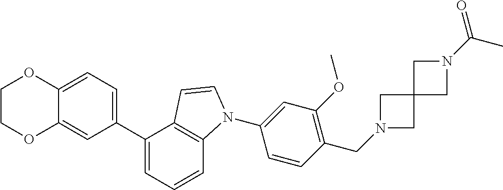 [SMILES: COc1cc(-n2ccc3c(-c4ccc5c(c4)OCCO5)cccc32)ccc1CN1CC2(C1)CN(C(C)=O)C2] |
| | 1-(6-((6-(4-(2,3-dihydrobenzo[b][1,4]dioxin-6-yl)-1H-indol-1-yl)-2- |
| | methoxypyridin-3-yl)methyl)-2,6-diazaspiro[3.3]heptan-2-yl)ethan-1- |
| | one |
| 15 | 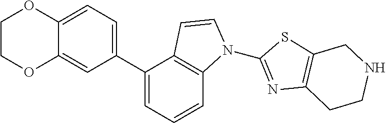 [SMILES: c1cc(-c2ccc3c(c2)OCCO3)c2ccn(-c3nc4c(s3)CNCC4)c2c1] |
| | 2-(4-(2,3-dihydrobenzo[b][1,4]dioxin-6-yl)-1H-indol-1-yl)-4,5,6,7- |
| | tetrahydrothiazolo[5,4-c]pyridine |
| 16 | 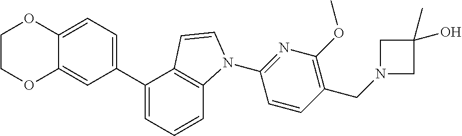 [SMILES: COc1nc(-n2ccc3c(-c4ccc5c(c4)OCCO5)cccc32)ccc1CN1CC(C)(O)C1] |
| | 1-((6-(4-(2,3-dihydrobenzo[b][1,4]dioxin-6-yl)-1H-indol-1-yl)-2- |
| | methoxypyridin-3-yl)methyl)-3-methylazetidin-3-ol |
| 17 | 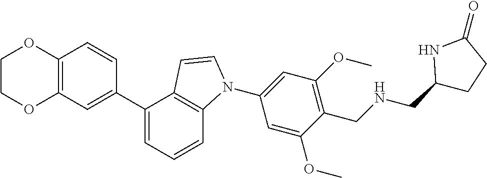 [SMILES: COc1cc(-n2ccc3c(-c4ccc5c(c4)OCCO5)cccc32)cc(OC)c1CNC[C@@H]1CCC(=O)N1] |
| | (S)-5-(((4-(4-(2,3-dihydrobenzo[b][1,4]dioxin-6-yl)indolin-1-yl)-2,6- |
| | dimethoxybenzyl)amino)methyl)pyrrolidin-2-one |
| 18 | 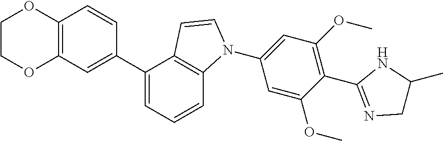 [SMILES: COc1cc(-n2ccc3c(-c4ccc5c(c4)OCCO5)cccc32)cc(OC)c1C1=NCC(C)N1] |
| | 4-(2,3-dihydrobenzo[b][1,4]dioxin-6-yl)-1-(3,5-dimethoxy-4-(5- |
| | methyl-4,5-dihydro-1H-imidazol-2-yl)phenyl)indoline |
| 19 | 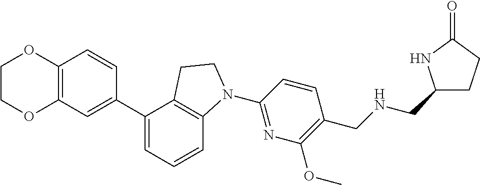 [SMILES: COc1nc(N2CCc3c(-c4ccc5c(c4)OCCO5)cccc32)ccc1CNC[C@@H]1CCC(=O)N1] |
| | (S)-5-((((6-(4-(2,3-dihydrobenzo[b][1,4]dioxin-6-yl)indolin-1-yl)-2- |
| | methoxypyridin-3-yl)methyl)amino)methyl)pyrrolidin-2-one |
| 20 | 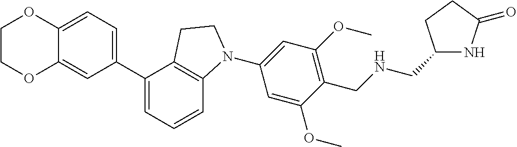 [SMILES: COc1cc(N2CCc3c(-c4ccc5c(c4)OCCO5)cccc32)cc(OC)c1CNC[C@@H]1CCC(=O)N1] |
| | (S)-5-(((4-(4-(2,3-dihydrobenzo[b][1,4]dioxin-6-yl)-1H-indol-1-yl)- |
| | 2,6-dimethoxybenzyl)amino)methyl)pyrrolidin-2-one |
| 21 | 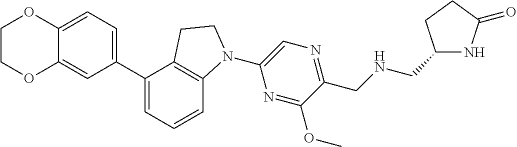 [SMILES: COc1nc(N2CCc3c(-c4ccc5c(c4)OCCO5)cccc32)cnc1CNC[C@@H]1CCC(=O)N1] |
| | (S)-5-((((5-(4-(2,3-dihydrobenzo[b][1,4]dioxin-6-yl)-1/-indol-1-yl)- |
| | 3-methoxypyrazin-2-yl)methyl)amino)methyl)pyrrolidin-2-one |
| 22 | 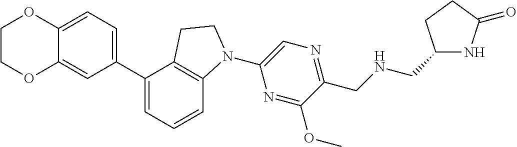 [SMILES: COc1nc(N2CCc3c(-c4ccc5c(c4)OCCO5)cccc32)cnc1CNC[C@@H]1CCC(=O)N1] |
| | (S)-5-((((5-(4-(2,3-dihydrobenzo[b][1,4]dioxin-6-yl)indolin-1-yl)-3- |
| | methoxypyrazin-2-yl)methyl)amino)methyl)pyrrolidin-2-one |
| 23 | 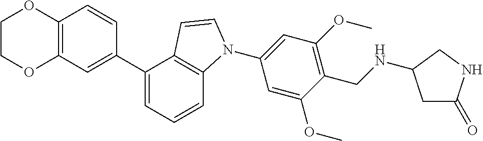 [SMILES: COc1cc(-n2ccc3c(-c4ccc5c(c4)OCCO5)cccc32)cc(OC)c1CNC1CNC(=O)C1] |
| | 4-((4-(4-(2,3-dihydrobenzo[b][1,4]dioxin-6-yl)-1H-indol-1-yl)-2,6- |
| | dimethoxybenzyl)amino)pyrrolidin-2-one |
| 24 | 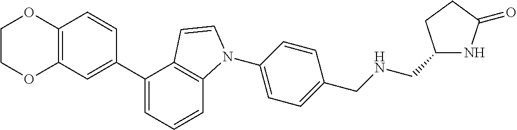 [SMILES: O=C1CC[C@@H](CNCc2ccc(-n3ccc4c(-c5ccc6c(c5)OCCO6)cccc43)cc2)N1] |
| | (S)-5-(((4-(4-(2,3-dihydrobenzo[b][1,4]dioxin-6-yl)-1H-indol-1- |
| | yl)benzyl)amino)methyl)pyrrolidin-2-one |
| 25 | 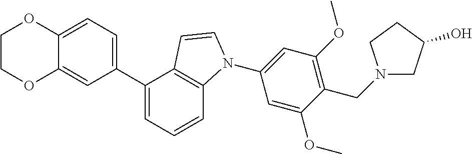 [SMILES: COc1cc(-n2ccc3c(-c4ccc5c(c4)OCCO5)cccc32)cc(OC)c1CN1CC[C@H](O)C1] |
| | (S)-1-(4-(4-(2,3-dihydrobenzo[b][1,4]dioxin-6-yl)-1H-indol-1-yl)-2,6- |
| | dimethoxybenzyl)pyrrolidin-3-ol |
| 26 | 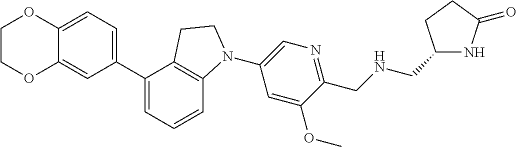 [SMILES: COc1cc(N2CCc3c(-c4ccc5c(c4)OCCO5)cccc32)cnc1CNC[C@@H]1CCC(=O)N1] |
| | (S)-5-((((5-(4-(2,3-dihydrobenzo[b][1,4]dioxin-6-yl)-1H-indol-1-yl)- |
| | 3-methoxypyridin-2-yl)methyl)amino)methyl)pyrrolidin-2-one |
| 27 | 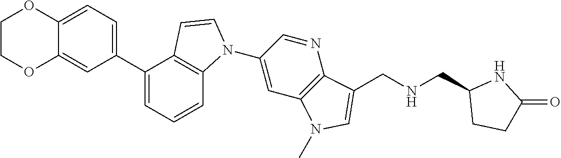 [SMILES: Cn1cc(CNC[C@@H]2CCC(=O)N2)c2ncc(-n3ccc4c(-c5ccc6c(c5)OCCO6)cccc43)cc21] |
| | (S)-5-((((6-(4-(2,3-dihydrobenzo[b][1,4]dioxin-6-yl)-1H-indol-1-yl)- |
| | 1-methyl-1H-pyrrolo[3,2-b]pyridin-3- |
| | yl)methyl)amino)methyl)pyrrolidin-2-one |
| 28 | 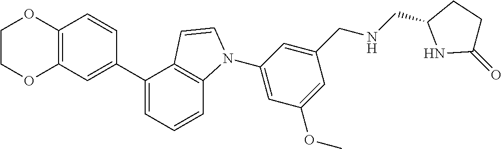 [SMILES: COc1cc(CNC[C@@H]2CCC(=O)N2)cc(-n2ccc3c(-c4ccc5c(c4)OCCO5)cccc32)c1] |
| | (S)-5-(((3-(4-(2,3-dihydrobenzo[b][1,4]dioxin-6-yl)-1H-indol-1-yl)-5- |
| | methoxybenzyl)amino)methyl)pyrrolidin-2-one |
| 29 | 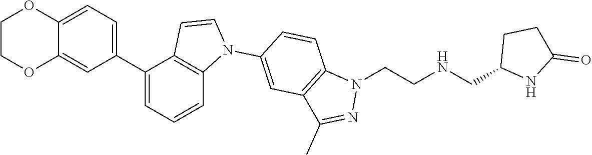 [SMILES: Cc1nn(CCNC[C@@H]2CCC(=O)N2)c2ccc(-n3ccc4c(-c5ccc6c(c5)OCCO6)cccc43)cc12] |
| | (S)-5-(((2-(5-(4-(2,3-dihydrobenzo[b][1,4]dioxin-6-yl)-1H-indol-1- |
| | yl)-3-methyl-1H-indazol-1-yl)ethyl)amino)methyl)pyrrolidin-2-one |
| 30 | 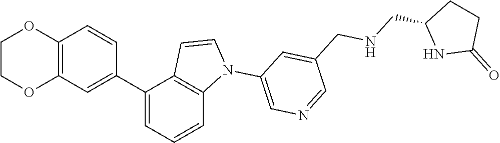 [SMILES: O=C1CC[C@@H](CNCc2cncc(-n3ccc4c(-c5ccc6c(c5)OCCO6)cccc43)c2)N1] |
| | (S)-5-((((5-(4-(2,3-dihydrobenzo[b][1,4]dioxin-6-yl)-1H-indol-1- |
| | yl)pyridin-3-yl)methyl)amino)methyl)pyrrolidin-2-one |
| 31 | 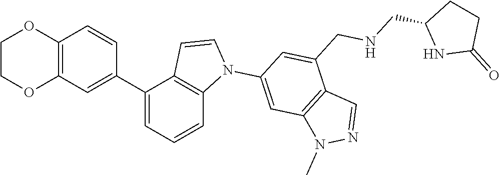 [SMILES: Cn1ncc2c(CNC[C@@H]3CCC(=O)N3)cc(-n3ccc4c(-c5ccc6c(c5)OCCO6)cccc43)cc21] |
| | (S)-5-((((6-(4-(2,3-dihydrobenzo[b][1,4]dioxin-6-yl)-1H-indol-1-yl)- |
| | 1-methyl-1H-indazol-4-yl)methyl)amino)methyl)pyrrolidin-2-one |
| 32 | 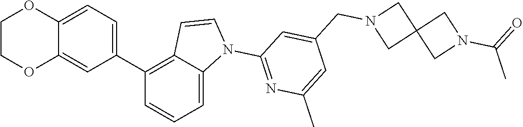 [SMILES: CC(=O)N1CC2(CN(Cc3cc(C)nc(-n4ccc5c(-c6ccc7c(c6)OCCO7)cccc54)c3)C2)C1] |
| | 1-(6-((2-(4-(2,3-dihydrobenzo[b][1,4]dioxin-6-yl)-1H-indol-1-yl)-6- |
| | methylpyridin-4-yl)methyl)-2,6-diazaspiro[3.3]heptan-2-yl)ethan-1- |
| | one |
| 33 | 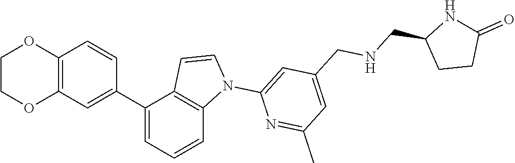 [SMILES: Cc1cc(CNC[C@@H]2CCC(=O)N2)cc(-n2ccc3c(-c4ccc5c(c4)OCCO5)cccc32)n1] |
| | (S)-5-((((2-(4-(2,3-dihydrobenzo[b][1,4]dioxin-6-yl)-1H-indol-1-yl)- |
| | 6-methylpyridin-4-yl)methyl)amino)methyl)pyrrolidin-2-one |
| 34 | 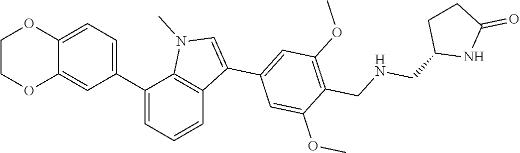 [SMILES: COc1cc(-c2cn(C)c3c(-c4ccc5c(c4)OCCO5)cccc23)cc(OC)c1CNC[C@@H]1CCC(=O)N1] |
| | (S)-5-(((4-(7-(2,3-dihydrobenzo[b][1,4]dioxin-6-yl)-1-methyl-1H- |
| | indol-3-yl)-2,6-dimethoxybenzyl)amino)methyl)pyrrolidin-2-one |
| 35 | 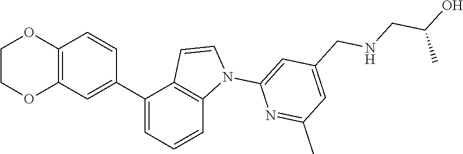 [SMILES: Cc1cc(CNC[C@@H](C)O)cc(-n2ccc3c(-c4ccc5c(c4)OCCO5)cccc32)n1] |
| | (R)-1-(((2-(4-(2,3-dihydrobenzo[b][1,4]dioxin-6-yl)-1H-indol-1-yl)-6- |
| | methylpyridin-4-yl)methyl)amino)propan-2-ol |
| 36 | 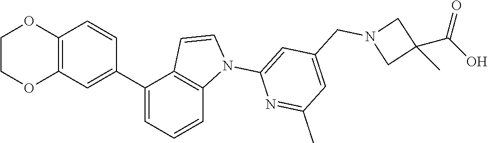 [SMILES: Cc1cc(CN2CC(C)(C(=O)O)C2)cc(-n2ccc3c(-c4ccc5c(c4)OCCO5)cccc32)n1] |
| | 1-((2-(4-(2,3-dihydrobenzo[b][1,4]dioxin-6-yl)-1H-indol-1-yl)-6- |
| | methylpyridin-4-yl)methyl)-3-methylazetidine-3-carboxylic acid |
| 37 | 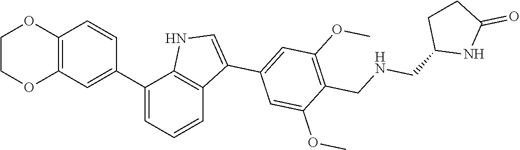 [SMILES: COc1cc(-c2c[nH]c3c(-c4ccc5c(c4)OCCO5)cccc23)cc(OC)c1CNC[C@@H]1CCC(=O)N1] |
| | (S)-5-(((4-(7-(2,3-dihydrobenzo[b][1,4]dioxin-6-yl)-1H-indol-3-yl)- |
| | 2,6-dimethoxybenzyl)amino)methyl)pyrrolidin-2-one |
| 38 | 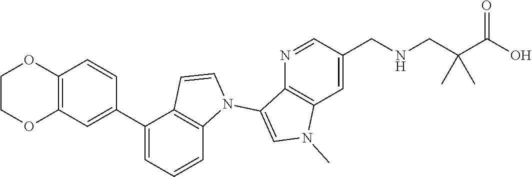 [SMILES: Cn1cc(-n2ccc3c(-c4ccc5c(c4)OCCO5)cccc32)c2ncc(CNCC(C)(C)C(=O)O)cc21] |
| | 3-(((3-(4-(2,3-dihydrobenzo[b][1,4]dioxin-6-yl)-1H-indol-1-yl)-1- |
| | methyl-1H-pyrrolo[3,2-b]pyridin-6-yl)methyl)amino)-2,2- |
| | dimethylpropanoic acid |
| 39 | 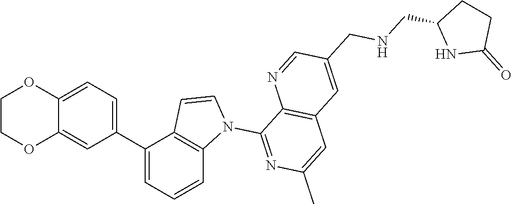 [SMILES: Cc1cc2cc(CNC[C@@H]3CCC(=O)N3)cnc2c(-n2ccc3c(-c4ccc5c(c4)OCCO5)cccc32)n1] |
| | (S)-5-((((8-(4-(2,3-dihydrobenzo[b][1,4]dioxin-6-yl)-1H-indol-1-yl)- |
| | 6-methyl-1,7-naphthyridin-3-yl)methyl)amino)methyl)pyrrolidin-2- |
| | one |
| 40 | 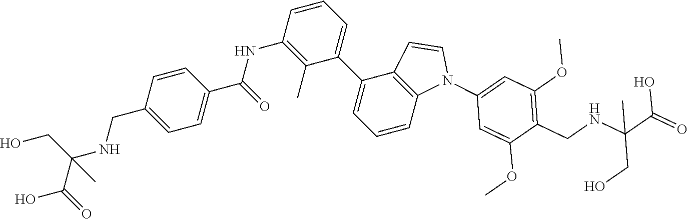 [SMILES: COc1cc(-n2ccc3c(-c4cccc(NC(=O)c5ccc(CNC(C)(CO)C(=O)O)cc5)c4C)cccc32)cc(OC)c1CNC(C)(CO)C(=O)O] |
| | 2-((4-((3-(1-(4-(((2-carboxy-1-hydroxypropan-2-yl)amino)methyl)- |
| | 3,5-dimethoxyphenyl)-1H-indol-4-yl)-2- |
| | methylphenyl)carbamoyl)benzyl)amino)-3-hydroxy-2- |
| | methylpropanoic acid |
| 41 | 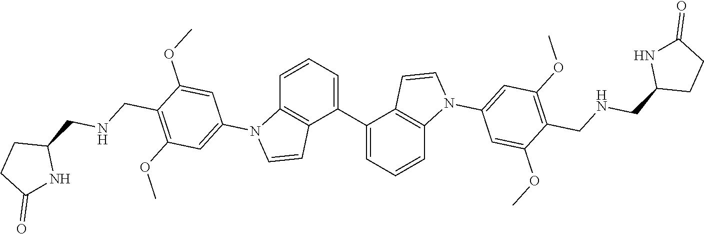 [SMILES: COc1cc(-n2ccc3c(-c4cccc5c4ccn5-c4cc(OC)c(CNC[C@@H]5CCC(=O)N5)c(OC)c4)cccc32)cc(OC)c1CNC[C@@H]1CCC(=O)N1] |
| | (S)-5-(((4-(4-(1-(3,5-dimethoxy-4-(((((S)-5-oxopyrrolidin-2- |
| | yl)methyl)amino)methyl)phenyl)-1H-indol-4-yl)indolin-1-yl)-2,6- |
| | dimethoxybenzyl)amino)methyl)pyrrolidin-2-one |
| 42 | 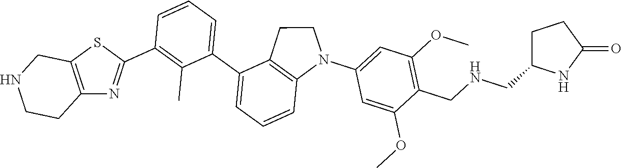 [SMILES: COc1cc(N2CCc3c(-c4cccc(-c5nc6c(s5)CNCC6)c4C)cccc32)cc(OC)c1CNC[C@@H]1CCC(=O)N1] |
| | (S)-5-(((2,6-dimethoxy-4-(4-(2-methyl-3-(4,5,6,7- |
| | tetrahydrothiazolo[5,4-c]pyridin-2-yl)phenyl)indolin-1- |
| | yl)benzyl)amino)methyl)pyrrolidin-2-one |
| 43 | 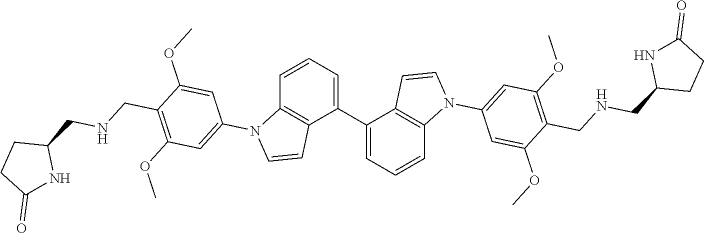 [SMILES: COc1cc(-n2ccc3c(-c4cccc5c4ccn5-c4cc(OC)c(CNC[C@@H]5CCC(=O)N5)c(OC)c4)cccc32)cc(OC)c1CNC[C@@H]1CCC(=O)N1] |
| | (5S,5′S)-5,5′-((((1H, l′H-[4,4′-biindole]-1,1′-diylbis(2,6-dimethoxy- |
| | 4,1- |
| | phenylene))bis(methylene))bis(azanediyl))bis(methylene))bis(pyrrolid |
| | in-2-one) |
| 44 | 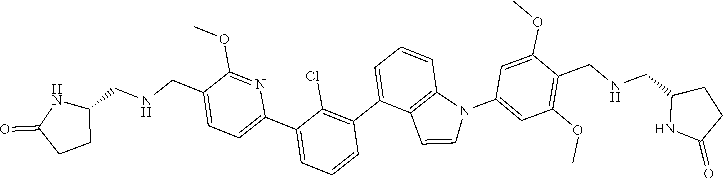 |
| | (S)-5-((((6-(2-chloro-3-(1-(3,5-dimethoxy-4-(((((S)-5-oxopyrrolidin- |
| | 2-yl)methyl)amino)methyl)phenyl)-1H-indol-4-yl)phenyl)-2- |
| | methoxypyridin-3-yl)methyl)amino)methyl)pyrrolidin-2-one |
| 45 | 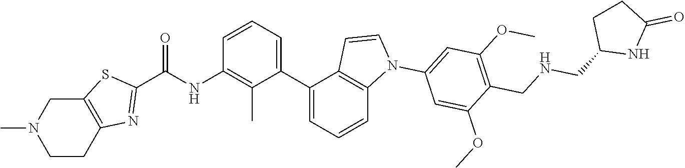 [SMILES: COc1cc(-n2ccc3c(-c4cccc(NC(=O)c5nc6c(s5)CN(C)CC6)c4C)cccc32)cc(OC)c1CNC[C@@H]1CCC(=O)N1] |
| | (S)-N-(3-(1-(3,5-dimethoxy-4-((((5-oxopyrrolidin-2- |
| | yl)methyl)amino)methyl)phenyl)-1H-indol-4-yl)-2-methylphenyl)-5- |
| | methyl-4,5,6,7-tetrahydrothiazolo[5,4-c]pyridine-2-carboxamide |
| 46 | 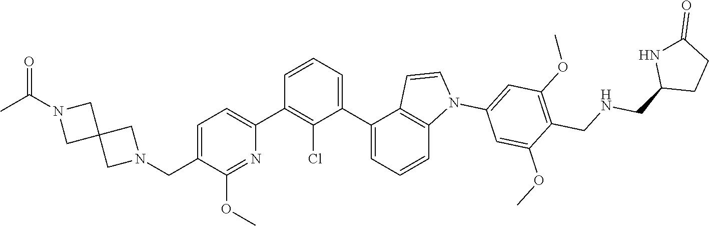 [SMILES: COc1cc(-n2ccc3c(-c4cccc(-c5ccc(CN6CC7(C6)CN(C(C)=O)C7)c(OC)n5)c4Cl)cccc32)cc(OC)c1CNC[C@@H]1CCC(=O)N1] |
| | (S)-5-(((4-(4-(3-(5-((6-acetyl-2,6-diazaspiro[3.3]heptan-2-yl)methyl)- |
| | 6-methoxypyridin-2-yl)-2-chlorophenyl)-1H-indol-1-yl)-2,6- |
| | dimethoxybenzyl)amino)methyl)pyrrolidin-2-one |
| 47 | 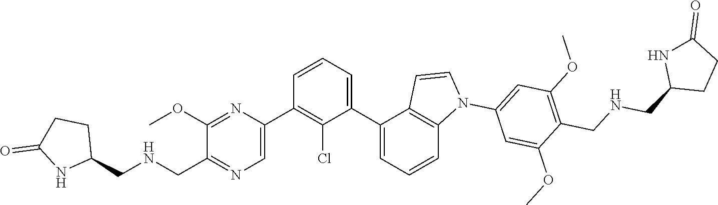 [SMILES: COc1cc(-n2ccc3c(-c4cccc(-c5cnc(CNC[C@@H]6CCC(=O)N6)c(OC)n5)c4Cl)cccc32)cc(OC)c1CNC[C@@H]1CCC(=O)N1] |
| | (S)-5-((((5-(2-chloro-3-(1-(3,5-dimethoxy-4-(((((S)-5-oxopyrrolidin- |
| | 2-yl)methyl)amino)methyl)phenyl)-1H-indol-4-yl)phenyl)-3- |
| | methoxypyrazin-2-yl)methyl)amino)methyl)pyrrolidin-2-one |
| 48 | 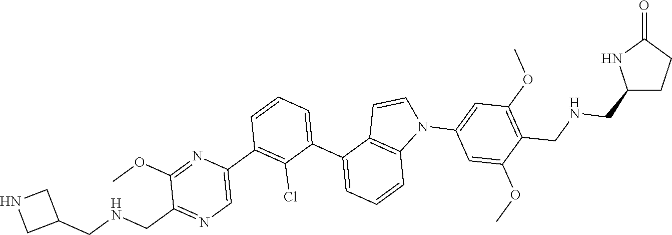 [SMILES: COc1cc(-n2ccc3c(-c4cccc(-c5cnc(CNCC6CNC6)c(OC)n5)c4Cl)cccc32)cc(OC)c1CNC[C@@H]1CCC(=O)N1] |
| | (S)-5-(((4-(4-(3-(5-(((azetidin-3-ylmethyl)amino)methyl)-6- |
| | methoxypyridin-2-yl)-2-chlorophenyl)-1H-indol-1-yl)-2,6- |
| | dimethoxybenzyl)amino)methyl)pyrrolidin-2-one |
| 49 | 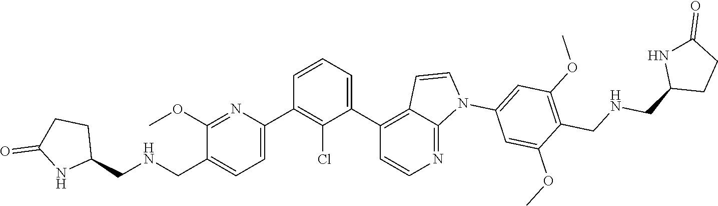 [SMILES: COc1cc(-n2ccc3c(-c4cccc(-c5ccc(CNC[C@@H]6CCC(=O)N6)c(OC)n5)c4Cl)ccnc32)cc(OC)c1CNC[C@@H]1CCC(=O)N1] |
| | (S)-5-((((6-(2-chloro-3-(1-(3,5-dimethoxy-4-(((((S)-5-oxopyrrolidin- |
| | 2-yl)methyl)amino)methyl)phenyl)-1H-pyrrolo[2,3-b]pyridin-4- |
| | yl)phenyl)-2-methoxypyridin-3-yl)methyl)amino)methyl)pyrrolidin-2- |
| | one |
| 50 | 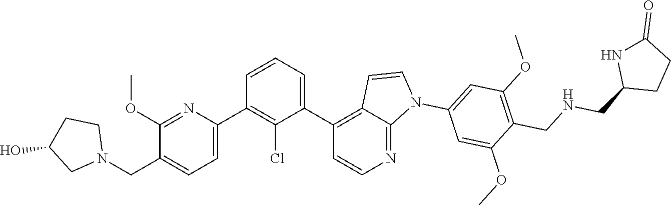 [SMILES: COc1cc(-n2ccc3c(-c4cccc(-c5ccc(CN6CC[C@@H](O)C6)c(OC)n5)c4Cl)ccnc32)cc(OC)c1CNC[C@@H]1CCC(=O)N1] |
| | (S)-5-(((4-(4-(2-chloro-3-(5-(((R)-3-hydroxypyrrolidin-1-yl)methyl)- |
| | 6-methoxypyridin-2-yl)phenyl)-1H-pyrrolo[2,3-b]pyridin-1-yl)-2,6- |
| | dimethoxybenzyl)amino)methyl)pyrrolidin-2-one |
| 51 | 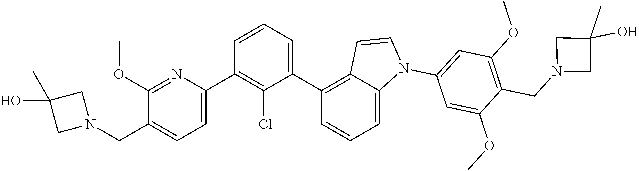 [SMILES: COc1cc(-n2ccc3c(-c4cccc(-c5ccc(CN6CC(C)(O)C6)c(OC)n5)c4Cl)cccc32)cc(OC)c1CN1CC(C)(O)C1] |
| | 1-((6-(2-chloro-3-(1-(4-((3-hydroxy-3-methylazetidin-1-yl)methyl)- |
| | 3,5-dimethoxyphenyl)-1H-indol-4-yl)phenyl)-2-methoxypyridin-3- |
| | yl)methyl)-3-methylazetidin-3-ol |
| 52 | 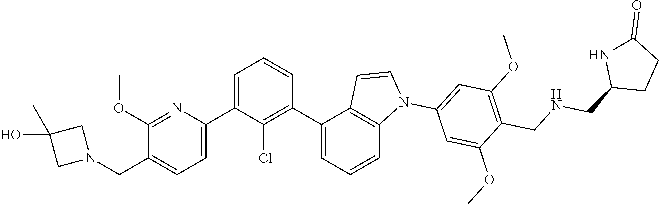 [SMILES: COc1cc(-n2ccc3c(-c4cccc(-c5ccc(CN6CC(C)(O)C6)c(OC)n5)c4Cl)cccc32)cc(OC)c1CNC[C@@H]1CCC(=O)N1] |
| | (S)-5-(((4-(4-(2-chloro-3-(5-((3-hydroxy-3-methylazetidin-1- |
| | yl)methyl)-6-methoxypyridin-2-yl)phenyl)-1H-indol-1-yl)-2,6- |
| | dimethoxybenzyl)amino)methyl)pyrrolidin-2-one |
| 53 | 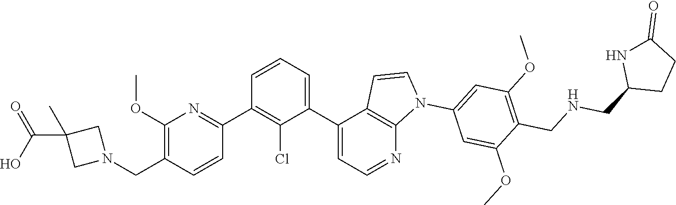 [SMILES: COc1cc(-n2ccc3c(-c4cccc(-c5ccc(CN6CC(C)(C(=O)O)C6)c(OC)n5)c4Cl)ccnc32)cc(OC)c1CNC[C@@H]1CCC(=O)N1] |
| | (S)-1-((6-(2-chloro-3-(1-(3,5-dimethoxy-4-((((5-oxopyrrolidin-2- |
| | yl)methyl)amino)methyl)phenyl)-1H-pyrrolo[2,3-b]pyridin-4- |
| | yl)phenyl)-2-methoxypyridin-3-yl)methyl)-3-methylazetidine-3- |
| | carboxylic acid |
| 54 | 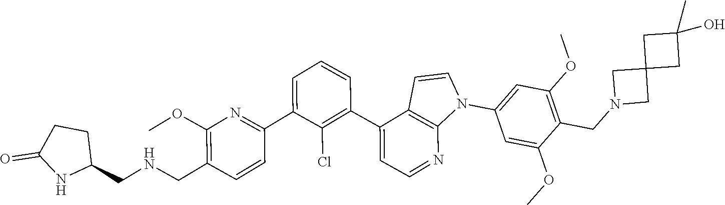 [SMILES: COc1cc(-n2ccc3c(-c4cccc(-c5ccc(CNC[C@@H]6CCC(=O)N6)c(OC)n5)c4Cl)ccnc32)cc(OC)c1CN1CC2(C1)CC(C)(O)C2] |
| | (S)-5-((((6-(2-chloro-3-(1-(4-((6-hydroxy-6-methyl-2- |
| | azaspiro[3.3]heptan-2-yl)methyl)-3,5-dimethoxyphenyl)-1H- |
| | pyrrolo[2,3-b]pyridin-4-yl)phenyl)-2-methoxypyridin-3- |
| | yl)methyl)amino)methyl)pyrrolidin-2-one |
| 55 | 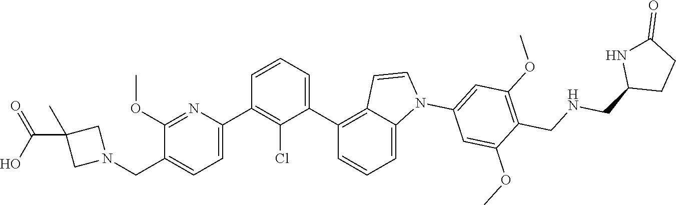 [SMILES: COc1cc(-n2ccc3c(-c4cccc(-c5ccc(CN6CC(C)(C(=O)O)C6)c(OC)n5)c4Cl)cccc32)cc(OC)c1CNC[C@@H]1CCC(=O)N1] |
| | (S)-1-((6-(2-chloro-3-(1-(3,5-dimethoxy-4-((((5-oxopyrrolidin-2- |
| | yl)methyl)amino)methyl)phenyl)-1H-indol-4-yl)phenyl)-2- |
| | methoxypyridin-3-yl)methyl)-3-methylazetidine-3-carboxylic acid |
| 56 | 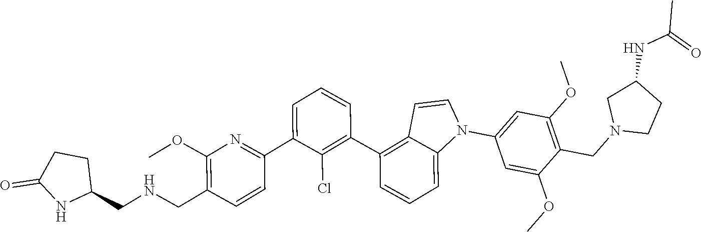 [SMILES: COc1cc(-n2ccc3c(-c4cccc(-c5ccc(CNC[C@@H]6CCC(=O)N6)c(OC)n5)c4Cl)cccc32)cc(OC)c1CN1CC[C@@H](NC(C)=O)C1] |
| | N-((R)-1-(4-(4-(2-chloro-3-(6-methoxy-5-(((((S)-5-oxopyrrolidin-2- |
| | yl)methyl)amino)methyl)pyridin-2-yl)phenyl)-1H-indol-1-yl)-2,6- |
| | dimethoxybenzyl)pyrrolidin-3-yl)acetamide |
| 57 | 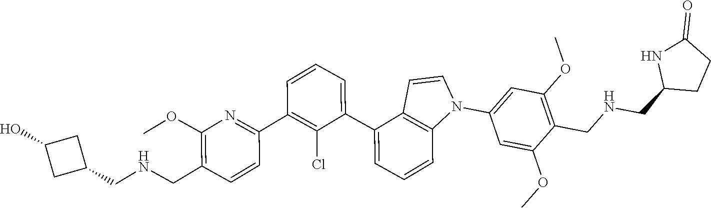 [SMILES: COc1cc(-n2ccc3c(-c4cccc(-c5ccc(CNC[C@H]6C[C@@H](O)C6)c(OC)n5)c4Cl)cccc32)cc(OC)c1CNC[C@@H]1CCC(=O)N1] |
| | (S)-5-(((4-(4-(2-chloro-3-(5-(((((18,3S)-3- |
| | hydroxycyclobutyl)methyl)amino)methyl)-6-methoxypyridin-2- |
| | yl)phenyl)-1H-indol-1-yl)-2,6- |
| | dimethoxybenzyl)amino)methyl)pyrrolidin-2-one |
| 58 | 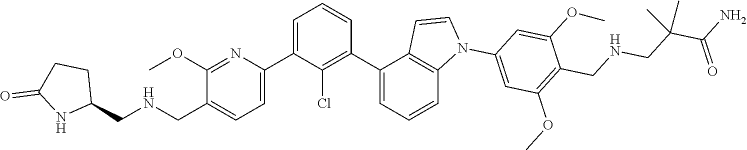 [SMILES: COc1cc(-n2ccc3c(-c4cccc(-c5ccc(CNC[C@@H]6CCC(=O)N6)c(OC)n5)c4Cl)cccc32)cc(OC)c1CNCC(C)(C)C(N)=O] |
| | (S)-3-((4-(4-(2-chloro-3-(6-methoxy-5-((((5-oxopyrrolidin-2- |
| | yl)methyl)amino)methyl)pyridin-2-yl)phenyl)-1H-indol-1-yl)-2,6- |
| | dimethoxybenzyl)amino)-2,2-dimethylpropanamide |
| 59 | 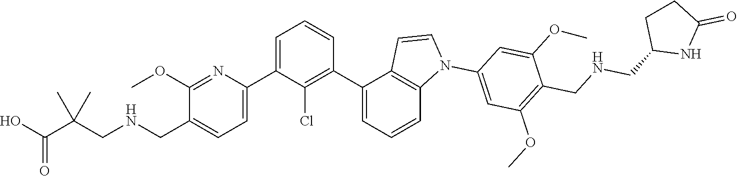 [SMILES: COc1cc(-n2ccc3c(-c4cccc(-c5ccc(CNCC(C)(C)C(=O)O)c(OC)n5)c4Cl)cccc32)cc(OC)c1CNC[C@@H]1CCC(=O)N1] |
| | (S)-3-(((6-(2-chloro-3-(1-(3,5-dimethoxy-4-((((5-oxopyrrolidin-2- |
| | yl)methyl)amino)methyl)phenyl)-1H-indol-4-yl)phenyl)-2- |
| | methoxypyridin-3-yl)methyl)amino)-2,2-dimethylpropanoic acid |
| 60 | 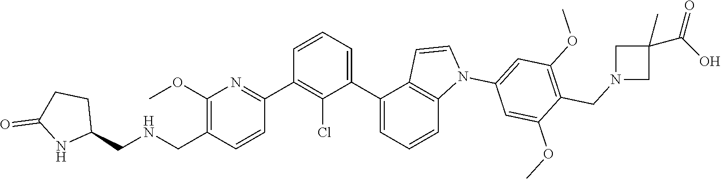 [SMILES: COc1cc(-n2ccc3c(-c4cccc(-c5ccc(CNC[C@@H]6CCC(=O)N6)c(OC)n5)c4Cl)cccc32)cc(OC)c1CN1CC(C)(C(=O)O)C1] |
| | (S)-1-(4-(4-(2-chloro-3-(6-methoxy-5-((((5-oxopyrrolidin-2- |
| | yl)methyl)amino)methyl)pyridin-2-yl)phenyl)-1H-indol-1-yl)-2,6- |
| | dimethoxybenzyl)-3-methylazetidine-3-carboxylic acid |
| 61 | 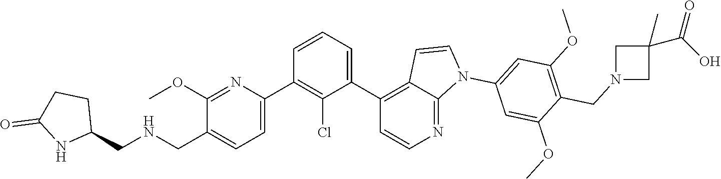 [SMILES: COc1cc(-n2ccc3c(-c4cccc(-c5ccc(CNC[C@@H]6CCC(=O)N6)c(OC)n5)c4Cl)ccnc32)cc(OC)c1CN1CC(C)(C(=O)O)C1] |
| | (S)-1-(4-(4-(2-chloro-3-(6-methoxy-5-((((5-oxopyrrolidin-2- |
| | yl)methyl)amino)methyl)pyridin-2-yl)phenyl)-1H-pyrrolo[2,3- |
| | b]pyridin-1-yl)-2,6-dimethoxybenzyl)-3-methylazetidine-3-carboxylic |
| | acid |
| 62 | 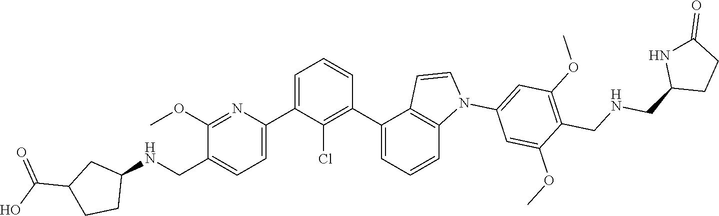 [SMILES: COc1cc(-n2ccc3c(-c4cccc(-c5ccc(CN[C@H]6CCC(C(=O)O)C6)c(OC)n5)c4Cl)cccc32)cc(OC)c1CNC[C@@H]1CCC(=O)N1] |
| | (3S)-3-(((6-(2-chloro-3-(1-(3,5-dimethoxy-4-(((((S)-5-oxopyrrolidin- |
| | 2-yl)methyl)amino)methyl)phenyl)-1H-indol-4-yl)phenyl)-2- |
| | methoxypyridin-3-yl)methyl)amino)cyclopentane-1-carboxylic acid |
| 63 | 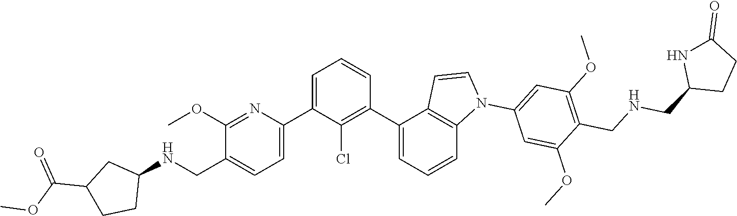 [SMILES: COC(=O)C1CC[C@H](NCc2ccc(-c3cccc(-c4cccc5c4ccn5-c4cc(OC)c(CNC[C@@H]5CCC(=O)N5)c(OC)c4)c3Cl)nc2OC)C1] |
| | methyl (3S)-3-(((6-(2-chloro-3-(1-(3,5-dimethoxy-4-(((((S)-5- |
| | oxopyrrolidin-2-yl)methyl)amino)methyl)phenyl)-1H-indol-4- |
| | yl)phenyl)-2-methoxypyridin-3-yl)methyl)amino)cyclopentane-1- |
| | carboxylate |
| 64 | 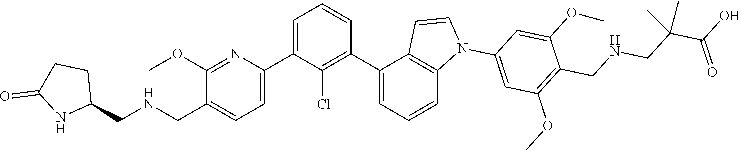 [SMILES: COc1cc(-n2ccc3c(-c4cccc(-c5ccc(CNC[C@@H]6CCC(=O)N6)c(OC)n5)c4Cl)cccc32)cc(OC)c1CNCC(C)(C)C(=O)O] |
| | (S)-3-((4-(4-(2-chloro-3-(6-methoxy-5-((((5-oxopyrrolidin-2- |
| | yl)methyl)amino)methyl)pyridin-2-yl)phenyl)-1H-indol-1-yl)-2,6- |
| | dimethoxybenzyl)amino)-2,2-dimethylpropanoic acid |
| 65 | 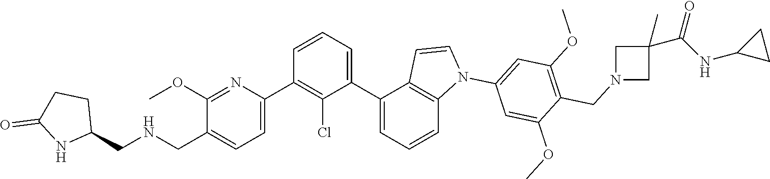 [SMILES: COc1cc(-n2ccc3c(-c4cccc(-c5ccc(CNC[C@@H]6CCC(=O)N6)c(OC)n5)c4Cl)cccc32)cc(OC)c1CN1CC(C)(C(=O)NC2CC2)C1] |
| | (S)-1-(4-(4-(2-chloro-3-(6-methoxy-5-((((5-oxopyrrolidin-2- |
| | yl)methyl)amino)methyl)pyridin-2-yl)phenyl)-1H-indol-1-yl)-2,6- |
| | dimethoxybenzyl)-N-cyclopropyl-3-methylazetidine-3-carboxamide |
| 66 | 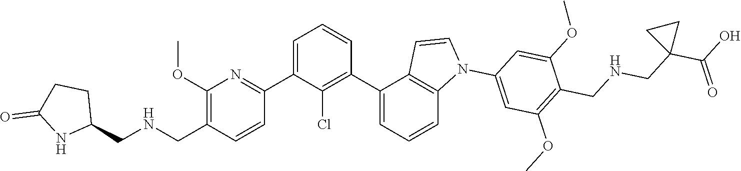 [SMILES: COc1cc(-n2ccc3c(-c4cccc(-c5ccc(CNC[C@@H]6CCC(=O)N6)c(OC)n5)c4Cl)cccc32)cc(OC)c1CNCC1(C(=O)O)CC1] |
| | (S)-1-(((4-(4-(2-chloro-3-(6-methoxy-5-((((5-oxopyrrolidin-2- |
| | yl)methyl)amino)methyl)pyridin-2-yl)phenyl)-1H-indol-1-yl)-2,6- |
| | dimethoxybenzyl)amino)methyl)cyclopropane-1-carboxylic acid |
| 67 | 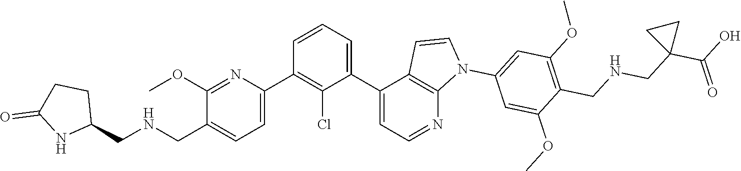 [SMILES: COc1cc(-n2ccc3c(-c4cccc(-c5ccc(CNC[C@@H]6CCC(=O)N6)c(OC)n5)c4Cl)ccnc32)cc(OC)c1CNCC1(C(=O)O)CC1] |
| | (S)-1-(((4-(4-(2-chloro-3-(6-methoxy-5-((((5-oxopyrrolidin-2- |
| | yl)methyl)amino)methyl)pyridin-2-yl)phenyl)-1H-pyrrolo[2,3- |
| | b]pyridin-1-yl)-2,6-dimethoxybenzyl)amino)methyl)cyclopropane-1- |
| | carboxylic acid |
| 68 | 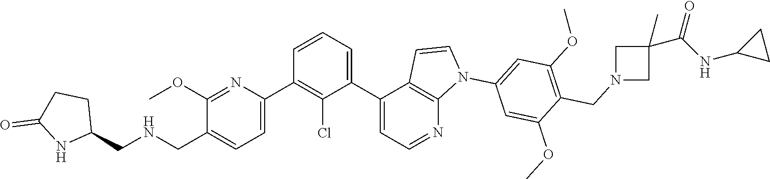 [SMILES: COc1cc(-n2ccc3c(-c4cccc(-c5ccc(CNC[C@@H]6CCC(=O)N6)c(OC)n5)c4Cl)ccnc32)cc(OC)c1CN1CC(C)(C(=O)NC2CC2)C1] |
| | (S)-1-(4-(4-(2-chloro-3-(6-methoxy-5-((((5-oxopyrrolidin-2- |
| | yl)methyl)amino)methyl)pyridin-2-yl)phenyl)-1H-pyrrolo[2,3- |
| | b]pyridin-1-yl)-2,6-dimethoxybenzyl)-N-cyclopropyl-3- |
| | methylazetidine-3-carboxamide |
| 69 | 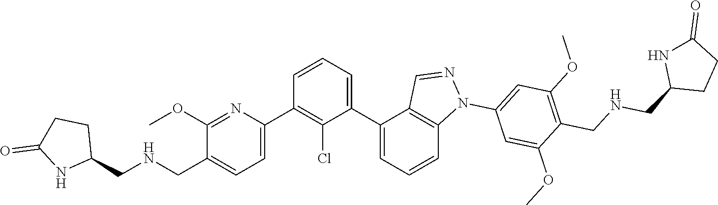 [SMILES: COc1cc(-n2ncc3c(-c4cccc(-c5ccc(CNC[C@@H]6CCC(=O)N6)c(OC)n5)c4Cl)cccc32)cc(OC)c1CNC[C@@H]1CCC(=O)N1] |
| | (S)-5-((((6-(2-chloro-3-(1-(3,5-dimethoxy-4-(((((S)-5-oxopyrrolidin- |
| | 2-yl)methyl)amino)methyl)phenyl)-1H-indazol-4-yl)phenyl)-2- |
| | methoxypyridin-3-yl)methyl)amino)methyl)pyrrolidin-2-one |
| 70 | 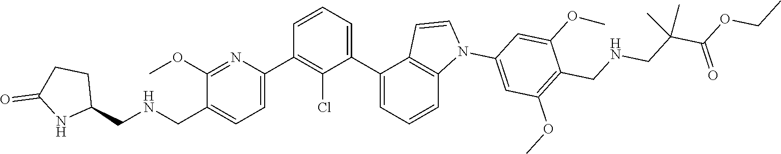 [SMILES: CCOC(=O)C(C)(C)CNCc1c(OC)cc(-n2ccc3c(-c4cccc(-c5ccc(CNC[C@@H]6CCC(=O)N6)c(OC)n5)c4Cl)cccc32)cc1OC] |
| | ethyl (S)-3-((4-(4-(2-chloro-3-(6-methoxy-5-((((5-oxopyrrolidin-2- |
| | yl)methyl)amino)methyl)pyridin-2-yl)phenyl)-1H-indol-1-yl)-2,6- |
| | dimethoxybenzyl)amino)-2,2-dimethylpropanoate |
| 71 | 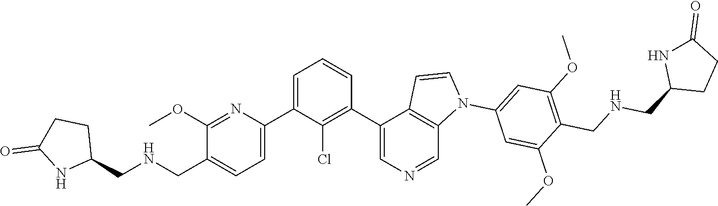 [SMILES: COc1cc(-n2ccc3c(-c4cccc(-c5ccc(CNC[C@@H]6CCC(=O)N6)c(OC)n5)c4Cl)cncc32)cc(OC)c1CNC[C@@H]1CCC(=O)N1] |
| | (S)-5-((((6-(2-chloro-3-(1-(3,5-dimethoxy-4-(((((S)-5-oxopyrrolidin- |
| | 2-yl)methyl)amino)methyl)phenyl)-1H-pyrrolo[2,3-c]pyridin-4- |
| | yl)phenyl)-2-methoxypyridin-3-yl)methyl)amino)methyl)pyrrolidin-2- |
| | one |
| 72 | 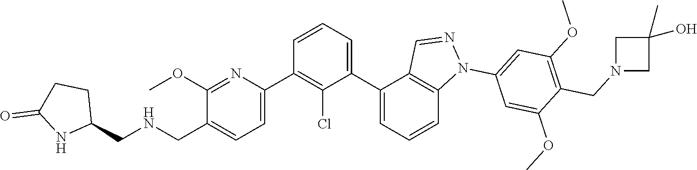 [SMILES: COc1cc(-n2ncc3c(-c4cccc(-c5ccc(CNC[C@@H]6CCC(=O)N6)c(OC)n5)c4Cl)cccc32)cc(OC)c1CN1CC(C)(O)C1] |
| | (S)-5-((((6-(2-chloro-3-(1-(4-((3-hydroxy-3-methylazetidin-1- |
| | yl)methyl)-3,5-dimethoxyphenyl)-1H-indazol-4-yl)phenyl)-2- |
| | methoxypyridin-3-yl)methyl)amino)methyl)pyrrolidin-2-one |
| 73 | 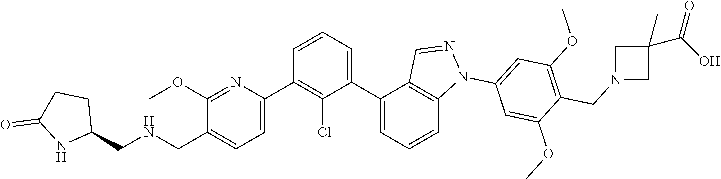 [SMILES: COc1cc(-n2ncc3c(-c4cccc(-c5ccc(CNC[C@@H]6CCC(=O)N6)c(OC)n5)c4Cl)cccc32)cc(OC)c1CN1CC(C)(C(=O)O)C1] |
| | (S)-1-(4-(4-(2-chloro-3-(6-methoxy-5-((((5-oxopyrrolidin-2- |
| | yl)methyl)amino)methyl)pyridin-2-yl)phenyl)-1H-indazol-1-yl)-2,6- |
| | dimethoxybenzyl)-3-methylazetidine-3-carboxylic acid |
| 74 | 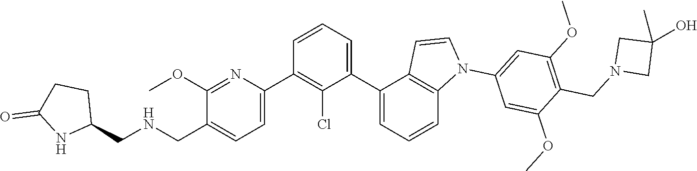 [SMILES: COc1cc(-n2ccc3c(-c4cccc(-c5ccc(CNC[C@@H]6CCC(=O)N6)c(OC)n5)c4Cl)cccc32)cc(OC)c1CN1CC(C)(O)C1] |
| | (S)-5-((((6-(2-chloro-3-(1-(4-((3-hydroxy-3-methylazetidin-1- |
| | yl)methyl)-3,5-dimethoxyphenyl)-1H-indol-4-yl)phenyl)-2- |
| | methoxypyridin-3-yl)methyl)amino)methyl)pyrrolidin-2-one |
| 75 | 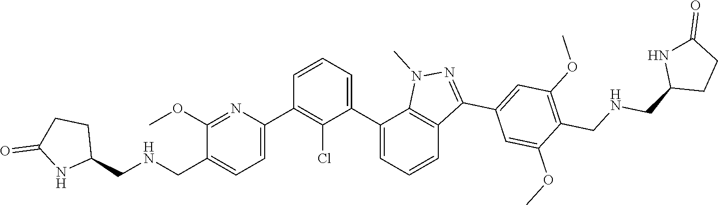 [SMILES: COc1cc(-c2nn(C)c3c(-c4cccc(-c5ccc(CNC[C@@H]6CCC(=O)N6)c(OC)n5)c4Cl)cccc23)cc(OC)c1CNC[C@@H]1CCC(=O)N1] |
| | (S)-5-((((6-(2-chloro-3-(3-(3,5-dimethoxy-4-(((((S)-5-oxopyrrolidin- |
| | 2-yl)methyl)amino)methyl)phenyl)-1-methyl-1H-indazol-7- |
| | yl)phenyl)-2-methoxypyridin-3-yl)methyl)amino)methyl)pyrrolidin-2- |
| | one |
| 76 | 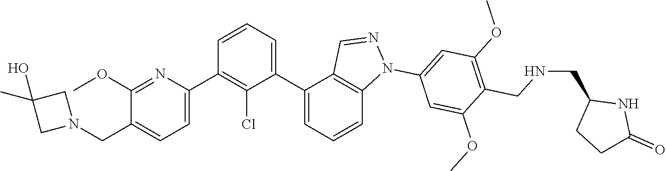 [SMILES: COc1cc(-n2ncc3c(-c4cccc(-c5ccc(CN6CC(C)(O)C6)c(OC)n5)c4Cl)cccc32)cc(OC)c1CNC[C@@H]1CCC(=O)N1] |
| | (S)-5-(((4-(4-(2-chloro-3-(5-((3-hydroxy-3-methylazetidin-1- |
| | yl)methyl)-6-methoxypyridin-2-yl)phenyl)-1H-indazol-1-yl)-2,6- |
| | dimethoxybenzyl)amino)methyl)pyrrolidin-2-one |
| 77 | 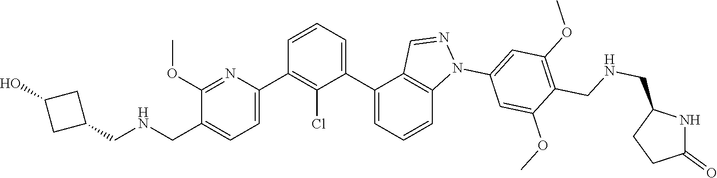 [SMILES: COc1cc(-n2ncc3c(-c4cccc(-c5ccc(CNC[C@H]6C[C@@H](O)C6)c(OC)n5)c4Cl)cccc32)cc(OC)c1CNC[C@@H]1CCC(=O)N1] |
| | (S)-5-(((4-(4-(2-chloro-3-(5-((((cis-3- |
| | hydroxycyclobutyl)methyl)amino)methyl)-6-methoxypyridin-2- |
| | yl)phenyl)-1H-indazol-1-yl)-2,6- |
| | dimethoxybenzyl)amino)methyl)pyrrolidin-2-one |
| 78 | 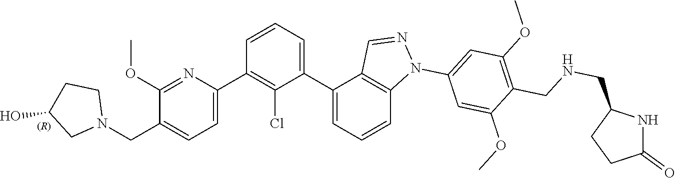 |
| | (S)-5-(((4-(4-(2-chloro-3-(5-(((R)-3-hydroxypyrrolidin-1-yl)methyl)- |
| | 6-methoxypyridin-2-yl)phenyl)-1H-indazol-1-yl)-2,6- |
| | dimethoxybenzyl)amino)methyl)pyrrolidin-2-one |
| 79 | 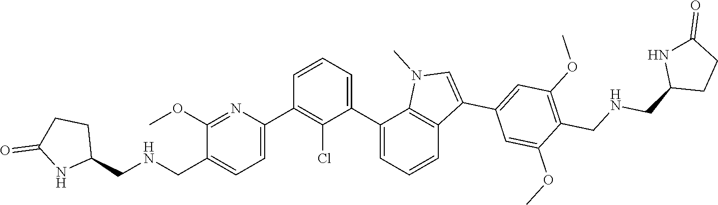 [SMILES: COc1cc(-c2cn(C)c3c(-c4cccc(-c5ccc(CNC[C@@H]6CCC(=O)N6)c(OC)n5)c4Cl)cccc23)cc(OC)c1CNC[C@@H]1CCC(=O)N1] |
| | (S)-5-((((6-(2-chloro-3-(3-(3,5-dimethoxy-4-(((((S)-5-oxopyrrolidin- |
| | 2-yl)methyl)amino)methyl)phenyl)-1-methyl-1H-indol-7-yl)phenyl)- |
| | 2-methoxypyridin-3-yl)methyl)amino)methyl)pyrrolidin-2-one |
| 80 | 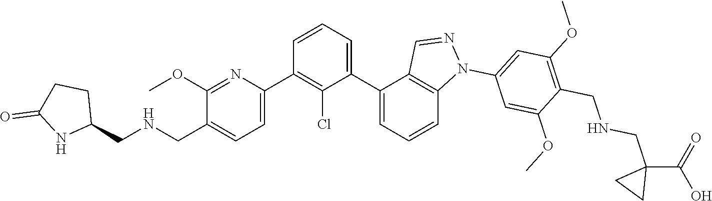 [SMILES: COc1cc(-n2ncc3c(-c4cccc(-c5ccc(CNC[C@@H]6CCC(=O)N6)c(OC)n5)c4Cl)cccc32)cc(OC)c1CNCC1(C(=O)O)CC1] |
| | (S)-1-(((4-(4-(2-chloro-3-(6-methoxy-5-((((5-oxopyrrolidin-2- |
| | yl)methyl)amino)methyl)pyridin-2-yl)phenyl)-1H-indazol-1-yl)-2,6- |
| | dimethoxybenzyl)amino)methyl)cyclopropane-1-carboxylic acid |
| 81 | 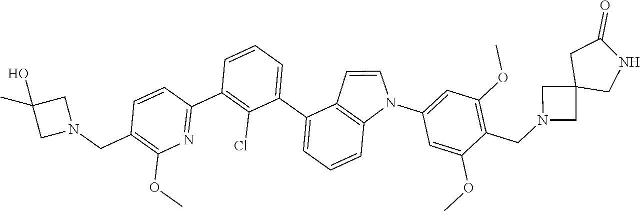 [SMILES: COc1cc(-n2ccc3c(-c4cccc(-c5ccc(CN6CC(C)(O)C6)c(OC)n5)c4Cl)cccc32)cc(OC)c1CN1CC2(CNC(=O)C2)C1] |
| | 2-(4-(4-(2-chloro-3-(5-((3-hydroxy-3-methylazetidin-1-yl)methyl)-6- |
| | methoxypyridin-2-yl)phenyl)-1H-indol-1-yl)-2,6-dimethoxybenzyl)- |
| | 2,6-diazaspiro[3.4]octan-7-one |
| 82 | 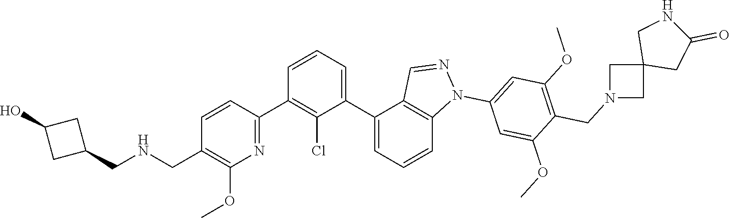 [SMILES: COc1cc(-n2ncc3c(-c4cccc(-c5ccc(CNC[C@H]6C[C@@H](O)C6)c(OC)n5)c4Cl)cccc32)cc(OC)c1CN1CC2(CNC(=O)C2)C1] |
| | 2-(4-(4-(2-chloro-3-(5-((((cis-3- |
| | hydroxycyclobutyl)methyl)amino)methyl)-6-methoxypyridin-2- |
| | yl)phenyl)-1H-indazol-1-yl)-2,6-dimethoxybenzyl)-2,6- |
| | diazaspiro[3.4]octan-7-one |
| 83 | 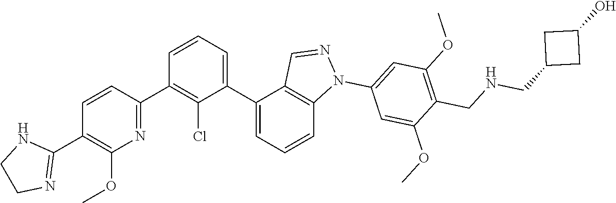 [SMILES: COc1cc(-n2ncc3c(-c4cccc(-c5ccc(C6=NCCN6)c(OC)n5)c4Cl)cccc32)cc(OC)c1CNC[C@H]1C[C@@H](O)C1] |
| | cis-3-(((4-(4-(2-chloro-3-(5-(4,5-dihydro-1H-imidazol-2-yl)-6- |
| | methoxypyridin-2-yl)phenyl)-1H-indazol-1-yl)-2,6- |
| | dimethoxybenzyl)amino)methyl)cyclobutan-1-ol |
| 84 | 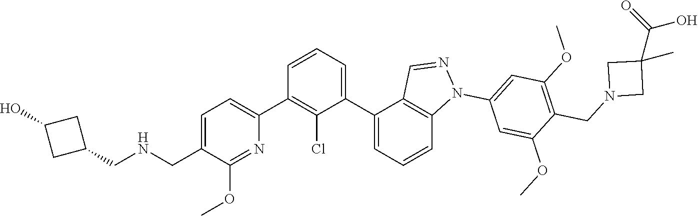 [SMILES: COc1cc(-n2ncc3c(-c4cccc(-c5ccc(CNC[C@H]6C[C@@H](O)C6)c(OC)n5)c4Cl)cccc32)cc(OC)c1CN1CC(C)(C(=O)O)C1] |
| | 1-(4-(4-(2-chloro-3-(5-((((cis-3- |
| | hydroxycyclobutyl)methyl)amino)methyl)-6-methoxypyridin-2- |
| | yl)phenyl)-1H-indazol-1-yl)-2,6-dimethoxybenzyl)-3- |
| | methylazetidine-3-carboxylic acid |
| 85 | 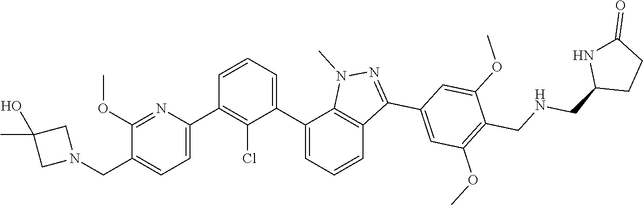 [SMILES: COc1cc(-c2nn(C)c3c(-c4cccc(-c5ccc(CN6CC(C)(O)C6)c(OC)n5)c4Cl)cccc23)cc(OC)c1CNC[C@@H]1CCC(=O)N1] |
| | (S)-5-(((4-(7-(2-chloro-3-(5-((3-hydroxy-3-methylazetidin-1- |
| | yl)methyl)-6-methoxypyridin-2-yl)phenyl)-1-methyl-1H-indazol-3- |
| | yl)-2,6-dimethoxybenzyl)amino)methyl)pyrrolidin-2-one |
| 86 | 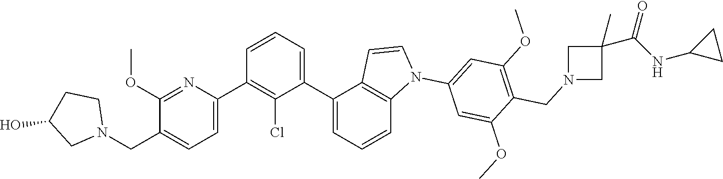 [SMILES: COc1cc(-n2ccc3c(-c4cccc(-c5ccc(CN6CC[C@@H](O)C6)c(OC)n5)c4Cl)cccc32)cc(OC)c1CN1CC(C)(C(=O)NC2CC2)C1] |
| | (R)-1-(4-(4-(2-chloro-3-(5-((3-hydroxypyrrolidin-1-yl)methyl)-6- |
| | methoxypyridin-2-yl)phenyl)-1H-indol-1-yl)-2,6-dimethoxybenzyl)- |
| | N-cyclopropyl-3-methylazetidine-3-carboxamide |
| 87 | 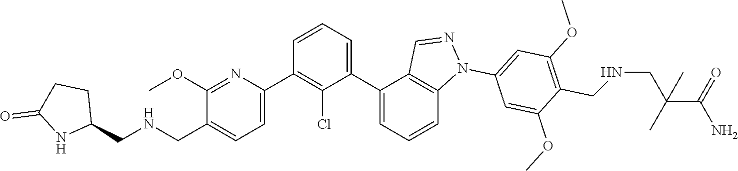 [SMILES: COc1cc(-n2ncc3c(-c4cccc(-c5ccc(CNC[C@@H]6CCC(=O)N6)c(OC)n5)c4Cl)cccc32)cc(OC)c1CNCC(C)(C)C(N)=O] |
| | (S)-3-((4-(4-(2-chloro-3-(6-methoxy-5-((((5-oxopyrrolidin-2- |
| | yl)methyl)amino)methyl)pyridin-2-yl)phenyl)-1H-indazol-1-yl)-2,6- |
| | dimethoxybenzyl)amino)-2,2-dimethylpropanamide |
| 88 | 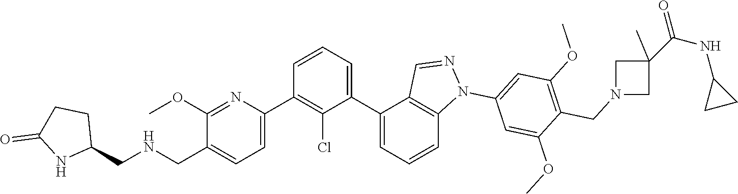 [SMILES: COc1cc(-n2ncc3c(-c4cccc(-c5ccc(CNC[C@@H]6CCC(=O)N6)c(OC)n5)c4Cl)cccc32)cc(OC)c1CN1CC(C)(C(=O)NC2CC2)C1] |
| | (S)-1-(4-(4-(2-chloro-3-(6-methoxy-5-((((5-oxopyrrolidin-2- |
| | yl)methyl)amino)methyl)pyridin-2-yl)phenyl)-1H-indazol-1-yl)-2,6- |
| | dimethoxybenzyl)-N-cyclopropyl-3-methylazetidine-3-carboxamide |
| 89 | 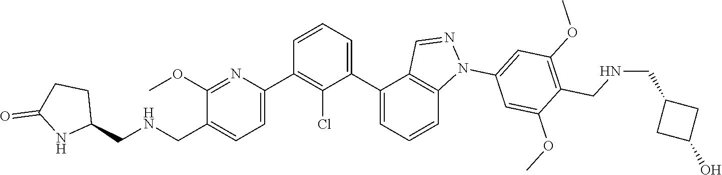 [SMILES: COc1cc(-n2ncc3c(-c4cccc(-c5ccc(CNC[C@@H]6CCC(=O)N6)c(OC)n5)c4Cl)cccc32)cc(OC)c1CNC[C@H]1C[C@@H](O)C1] |
| | (S)-5-((((6-(2-chloro-3-(1-(4-((((cis-3- |
| | hydroxycyclobutyl)methyl)amino)methyl)-3,5-dimethoxyphenyl)-1H- |
| | indazol-4-yl)phenyl)-2-methoxypyridin-3- |
| | yl)methyl)amino)methyl)pyrrolidin-2-one |
| 90 | 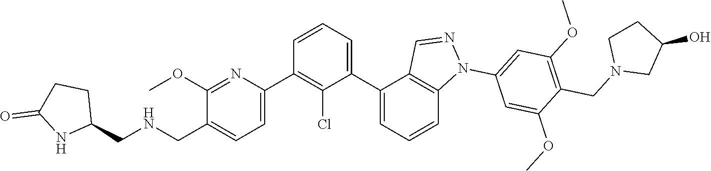 [SMILES: COc1cc(-n2ncc3c(-c4cccc(-c5ccc(CNC[C@@H]6CCC(=O)N6)c(OC)n5)c4Cl)cccc32)cc(OC)c1CN1CC[C@@H](O)C1] |
| | (S)-5-((((6-(2-chloro-3-(1-(4-(((R)-3-hydroxypyrrolidin-1-yl)methyl)- |
| | 3,5-dimethoxyphenyl)-1H-indazol-4-yl)phenyl)-2-methoxypyridin-3- |
| | yl)methyl)amino)methyl)pyrrolidin-2-one |
| 91 | 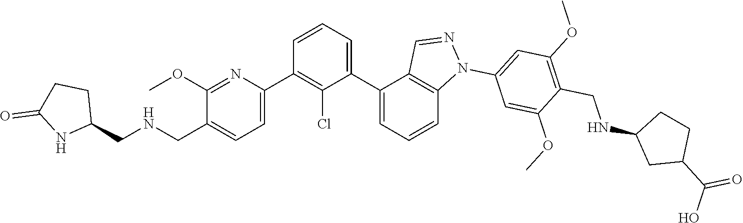 [SMILES: COc1cc(-n2ncc3c(-c4cccc(-c5ccc(CNC[C@@H]6CCC(=O)N6)c(OC)n5)c4Cl)cccc32)cc(OC)c1CN[C@H]1CCC(C(=O)O)C1] |
| | (38)-3-((4-(4-(2-chloro-3-(6-methoxy-5-(((((S)-5-oxopyrrolidin-2- |
| | yl)methyl)amino)methyl)pyridin-2-yl)phenyl)-1H-indazol-1-yl)-2,6- |
| | dimethoxybenzyl)amino)cyclopentane-1-carboxylic acid |
| 92 | 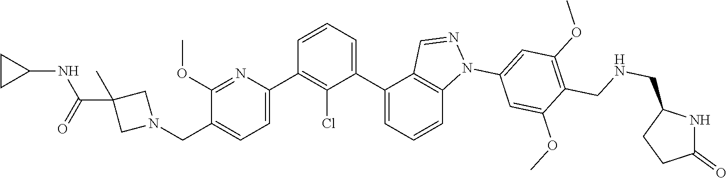 [SMILES: COc1cc(-n2ncc3c(-c4cccc(-c5ccc(CN6CC(C)(C(=O)NC7CC7)C6)c(OC)n5)c4Cl)cccc32)cc(OC)c1CNC[C@@H]1CCC(=O)N1] |
| | (S)-1-((6-(2-chloro-3-(1-(3,5-dimethoxy-4-((((5-oxopyrrolidin-2- |
| | yl)methyl)amino)methyl)phenyl)-1H-indazol-4-yl)phenyl)-2~ |
| | methoxypyridin-3-yl)methyl)-N-cyclopropyl-3-methylazetidine-3- |
| | carboxamide |
| 93 | 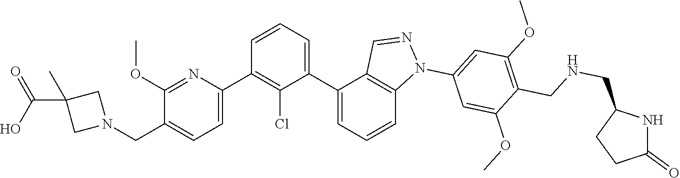 [SMILES: COc1cc(-n2ncc3c(-c4cccc(-c5ccc(CN6CC(C)(C(=O)O)C6)c(OC)n5)c4Cl)cccc32)cc(OC)c1CNC[C@@H]1CCC(=O)N1] |
| | (S)-1-((6-(2-chloro-3-(1-(3,5-dimethoxy-4-((((5-oxopyrrolidin~2- |
| | yl)methyl)amino)methyl)phenyl)-1H-indazol-4-yl)phenyl)-2- |
| | methoxypyridin-3-yl)methyl)-3-methylazetidine-3-carboxylic acid |
| 94 | 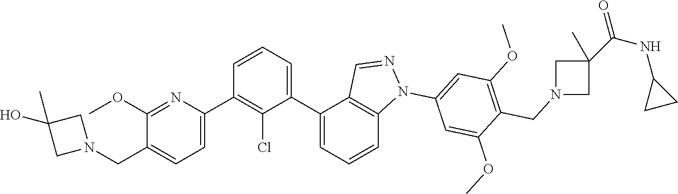 [SMILES: COc1cc(-n2ncc3c(-c4cccc(-c5ccc(CN6CC(C)(O)C6)c(OC)n5)c4Cl)cccc32)cc(OC)c1CN1CC(C)(C(=O)NC2CC2)C1] |
| | 1-(4-(4-(2-chloro-3-(5-((3-hydroxy-3-methylazetidin-1-yl)methyl)-6- |
| | methoxypyridin-2-yl)phenyl)-1H-indazol-1-yl)-2,6- |
| | dimethoxybenzyl)-N-cyclopropyl-3-methylazetidine-3-carboxamide |
| 95 | 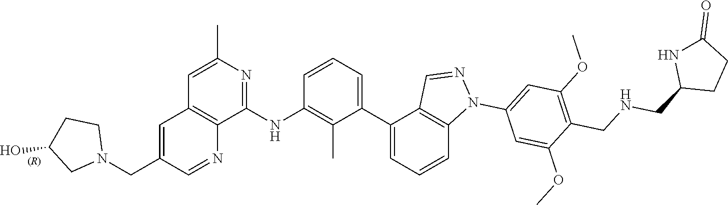 |
| | (S)-5-(((4-(4-(3-((3-(((R)-3-hydroxypyrrolidin-1-yl)methyl)-6-methyl- |
| | 1,7-naphthyridin-8-yl)amino)-2-methylphenyl)-1H-indazol-1-yl)-2,6- |
| | dimethoxybenzyl)amino)methyl)pyrrolidin-2-one |
| 96 | 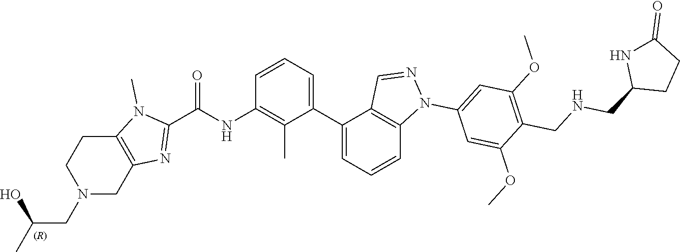 |
| | N-(3-(1-(3,5-dimethoxy-4-(((((S)-5-oxopyrrolidin-2- |
| | yl)methyl)amino)methyl)phenyl)-1H-indazol-4-yl)-2-methylphenyl)- |
| | 5-((R)-2-hydroxypropyl)-1-methyl-4,5,6,7-tetrahydro-1H- |
| | imidazo[4,5-c]pyridine-2-carboxamide |
| 97 | 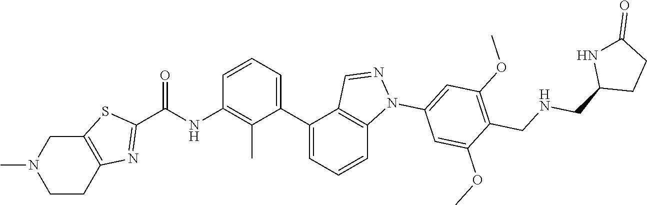 [SMILES: COc1cc(-n2ncc3c(-c4cccc(NC(=O)c5nc6c(s5)CN(C)CC6)c4C)cccc32)cc(OC)c1CNC[C@@H]1CCC(=O)N1] |
| | (S)-N-(3-(1-(3,5-dimethoxy-4-((((5-oxopyrrolidin-2- |
| | yl)methyl)amino)methyl)phenyl)-1H-indazol-4-yl)-2-methylphenyl)- |
| | 5-methyl-4,5,6,7-tetrahydrothiazolo[5,4-c]pyridine-2-carboxamide |
| 98 | 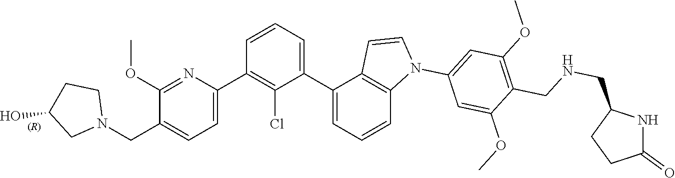 |
| | (S)-5-(((4-(4-(2-chloro-3-(5-(((R)-3-hydroxypyrrolidin-1-yl)methyl)- |
| | 6-methoxypyridin-2-yl)phenyl)-1H-indol-1-yl)-2,6- |
| | dimethoxybenzyl)amino)methyl)pyrrolidin-2-one |
| 99 | 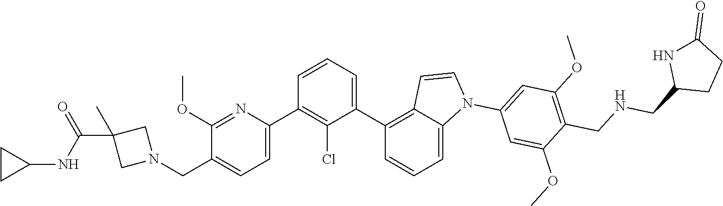 |
| | (S)-1-((6-(2-chloro-3-(1-(3,5-dimethoxy-4-((((5-oxopyrrolidin-2- |
| | yl)methyl)amino)methyl)phenyl)-1H-indol-4-yl)phenyl)-2- |
| | methoxypyridin-3-yl)methyl)-N-cyclopropyl-3-methylazetidine-3- |
| | carboxamide |
| 100 | 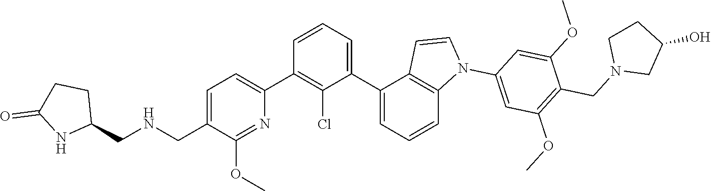 [SMILES: COc1cc(-n2ccc3c(-c4cccc(-c5ccc(CNC[C@@H]6CCC(=O)N6)c(OC)n5)c4Cl)cccc32)cc(OC)c1CN1CC[C@H](O)C1] |
| | (S)-5-((((6-(2-chloro-3-(1-(4-(((S)-3-hydroxypyrrolidin-1-yl)methyl)- |
| | 3,5-dimethoxyphenyl)-1H-indol-4-yl)phenyl)-2-methoxypyridin-3- |
| | yl)methyl)amino)methyl)pyrrolidin-2-one |
| 101 | 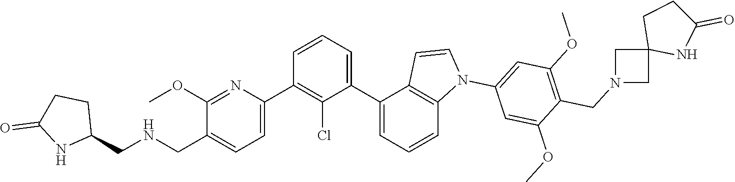 [SMILES: COc1cc(-n2ccc3c(-c4cccc(-c5ccc(CNC[C@@H]6CCC(=O)N6)c(OC)n5)c4Cl)cccc32)cc(OC)c1CN1CC2(CCC(=O)N2)C1] |
| | (S)-2-(4-(4-(2-chloro-3-(6-methoxy-5-((((5-oxopyrrolidin-2- |
| | yl)methyl)amino)methyl)pyridin-2-yl)phenyl)-1H-indol-1-yl)-2,6- |
| | dimethoxybenzyl)-2,5-diazaspiro[3.4]octan-6-one |
| 102 | 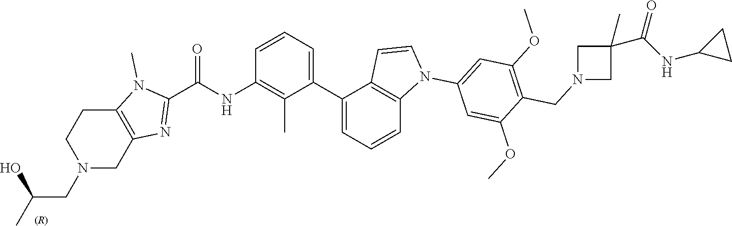 |
| | (R)-N-(3-(1-(4-((3-(cyclopropylcarbamoyl)-3-methylazetidin-1- |
| | yl)methyl)-3,5-dimethoxyphenyl)-1H-indol-4-yl)-2-methylphenyl)-5- |
| | (2-hydroxypropyl)-1-methyl-4,5,6,7-tetrahydro-1H-imidazo[4,5- |
| | clpyridine-2-carboxamide |
| 103 | 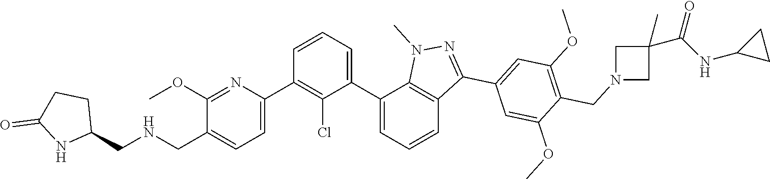 [SMILES: COc1cc(-c2nn(C)c3c(-c4cccc(-c5ccc(CNC[C@@H]6CCC(=O)N6)c(OC)n5)c4Cl)cccc23)cc(OC)c1CN1CC(C)(C(=O)NC2CC2)C1] |
| | (S)-1-(4-(7-(2-chloro-3-(6-methoxy-5-((((5-oxopyrrolidin-2- |
| | yl)methyl)amino)methyl)pyridin-2-yl)phenyl)-1-methyl-1H-indazol- |
| | 3-yl)-2,6-dimethoxybenzyl)-N-cyclopropyl-3-methylazetidine-3- |
| | carboxamide |
| 104 | 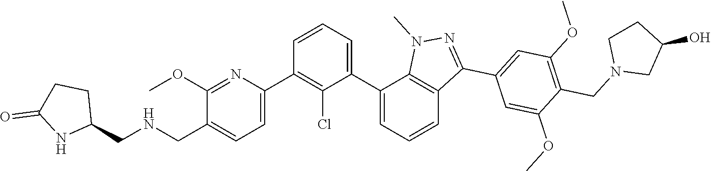 [SMILES: COc1cc(-c2nn(C)c3c(-c4cccc(-c5ccc(CNC[C@@H]6CCC(=O)N6)c(OC)n5)c4Cl)cccc23)cc(OC)c1CN1CC[C@@H](O)C1] |
| | (S)-5-((((6-(2-chloro-3-(3-(4-(((R)-3-hydroxypyrrolidin-1-yl)methyl)- |
| | 3,5-dimethoxyphenyl)-1-methyl-1H-indazol-7-yl)phenyl)-2- |
| | methoxypyridin-3-yl)methyl)amino)methyl)pyrrolidin-2-one |
| 105 | 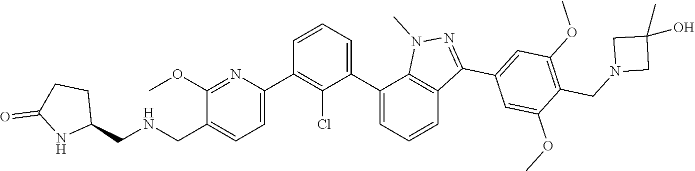 [SMILES: COc1cc(-c2nn(C)c3c(-c4cccc(-c5ccc(CNC[C@@H]6CCC(=O)N6)c(OC)n5)c4Cl)cccc23)cc(OC)c1CN1CC(C)(O)C1] |
| | (S)-5-((((6-(2-chloro-3-(3-(4-((3-hydroxy-3-methylazetidin-1- |
| | yl)methyl)-3,5-dimethoxyphenyl)-1-methyl-1H-indazol-7-yl)phenyl)- |
| | 2-methoxypyridin-3-yl)methyl)amino)methyl)pyrrolidin-2-one |
| 106 | 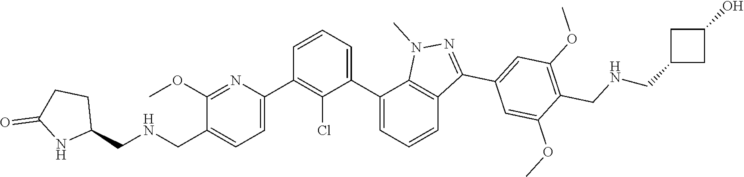 [SMILES: COc1cc(-c2nn(C)c3c(-c4cccc(-c5ccc(CNC[C@@H]6CCC(=O)N6)c(OC)n5)c4Cl)cccc23)cc(OC)c1CNC[C@H]1C[C@@H](O)C1] |
| | (S)-5-((((6-(2-chloro-3-(3-(4-(((((1s,3s)-3- |
| | hydroxycyclobutyl)methyl)amino)methyl)-3,5-dimethoxyphenyl)-1- |
| | methyl-1H-indazol-7-yl)phenyl)-2-methoxypyridin-3- |
| | yl)methyl)amino)methyl)pyrrolidin-2-one |
| 107 | 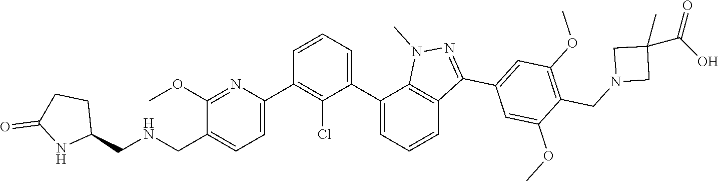 [SMILES: COc1cc(-c2nn(C)c3c(-c4cccc(-c5ccc(CNC[C@@H]6CCC(=O)N6)c(OC)n5)c4Cl)cccc23)cc(OC)c1CN1CC(C)(C(=O)O)C1] |
| | (S)-1-(4-(7-(2-chloro-3-(6-methoxy-5-((((5-oxopyrrolidin-2- |
| | yl)methyl)amino)methyl)pyridin-2-yl)phenyl)-1-methyl-1H-indazol- |
| | 3-yl)-2,6-dimethoxybenzyl)-3-methylazetidine-3-carboxylic acid |
| 108 | 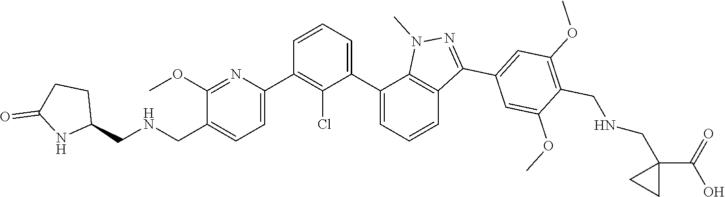 [SMILES: COc1cc(-c2nn(C)c3c(-c4cccc(-c5ccc(CNC[C@@H]6CCC(=O)N6)c(OC)n5)c4Cl)cccc23)cc(OC)c1CNCC1(C(=O)O)CC1] |
| | (S)-1-(((4-(7-(2-chloro-3-(6-methoxy-5-((((5-oxopyrrolidin-2- |
| | yl)methyl)amino)methyl)pyridin-2-yl)phenyl)-1-methyl-1H-indazol- |
| | 3-yl)-2,6-dimethoxybenzyl)amino)methyl)cyclopropane-1-carboxylic |
| | acid |
| 109 | 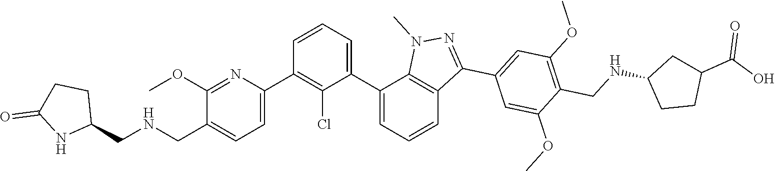 [SMILES: COc1cc(-c2nn(C)c3c(-c4cccc(-c5ccc(CNC[C@@H]6CCC(=O)N6)c(OC)n5)c4Cl)cccc23)cc(OC)c1CN[C@H]1CCC(C(=O)O)C1] |
| | (3S)-3-((4-(7-(2-chloro-3-(6-methoxy-5-(((((S)-5-oxopyrrolidin-2- |
| | yl)methyl)amino)methyl)pyridin-2-yl)phenyl)-1-methyl-1H-indazol- |
| | 3-yl)-2,6-dimethoxybenzyl)amino)cyclopentane-1-carboxylic acid |
| 110 | 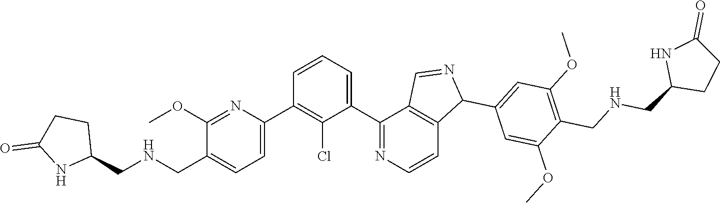 [SMILES: COc1cc(C2N=Cc3c2ccnc3-c2cccc(-c3ccc(CNC[C@@H]4CCC(=O)N4)c(OC)n3)c2Cl)cc(OC)c1CNC[C@@H]1CCC(=O)N1] |
| | (S)-5-((((6-(2-chloro-3-(1-(3,5-dimethoxy-4-(((((S)-5-oxopyrrolidin- |
| | 2-yl)methyl)amino)methyl)phenyl)-1H-pyrrolo[3,2-c]pyridin-4- |
| | yl)phenyl)-2-methoxypyridin-3-yl)methyl)amino)methyl)pyrrolidin-2- |
| | one |
| 111 | 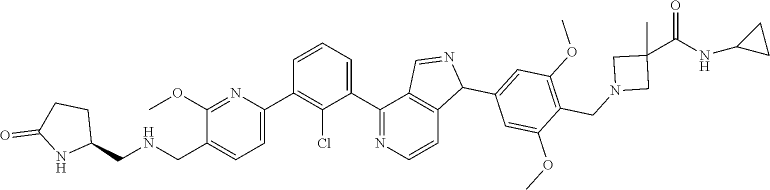 [SMILES: COc1cc(C2N=Cc3c2ccnc3-c2cccc(-c3ccc(CNC[C@@H]4CCC(=O)N4)c(OC)n3)c2Cl)cc(OC)c1CN1CC(C)(C(=O)NC2CC2)C1] |
| | (S)-1-(4-(4-(2-chloro-3-(6-methoxy-5-((((5-oxopyrrolidin-2- |
| | yl)methyl)amino)methyl)pyridin-2-yl)phenyl)-1H-pyrrolo[2,3- |
| | clpyridin-1-yl)-2,6-dimethoxybenzyl)-N-cyclopropyl-3- |
| | methylazetidine-3-carboxamide |
| 112 | 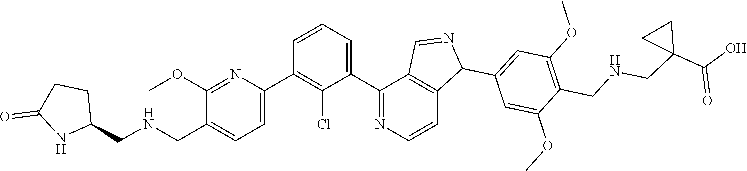 [SMILES: COc1cc(C2N=Cc3c2ccnc3-c2cccc(-c3ccc(CNC[C@@H]4CCC(=O)N4)c(OC)n3)c2Cl)cc(OC)c1CNCC1(C(=O)O)CC1] |
| | (S)-1-(((4-(4-(2-chloro-3-(6-methoxy-5-((((5-oxopyrrolidin-2- |
| | yl)methyl)amino)methyl)pyridin-2-yl)phenyl)-1H-pyrrolo[2,3- |
| | c]pyridin-1-yl)-2,6-dimethoxybenzyl)amino)methyl)cyclopropane-1- |
| | carboxylic acid |
| 113 | 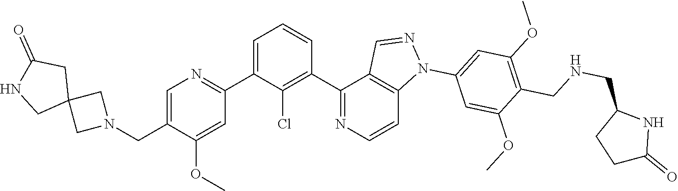 [SMILES: COc1cc(-c2cccc(-c3nccc4c3cnn4-c3cc(OC)c(CNC[C@@H]4CCC(=O)N4)c(OC)c3)c2Cl)ncc1CN1CC2(CNC(=O)C2)C1] |
| | (S)-2-((6-(2-chloro-3-(1-(3,5-dimethoxy-4-((((5-oxopyrrolidin-2- |
| | yl)methyl)amino)methyl)phenyl)-1H-indazol-4-yl)phenyl)-2- |
| | methoxypyridin-3-yl)methyl)-2,6-diazaspiro[3.4]octan-7-one |
| 114 | 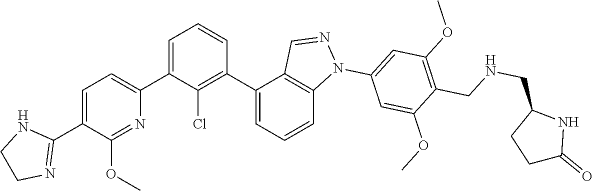 [SMILES: COc1cc(-n2ncc3c(-c4cccc(-c5ccc(C6=NCCN6)c(OC)n5)c4Cl)cccc32)cc(OC)c1CNC[C@@H]1CCC(=O)N1] |
| | (S)-5-(((4-(4-(2-chloro-3-(5-(4,5-dihydro-1H-imidazol-2-yl)-6- |
| | methoxypyridin-2-yl)phenyl)~1H-indazol-1-yl)-2,6- |
| | dimethoxybenzyl)amino)methyl)pyrrolidin-2-one |
| 115 | 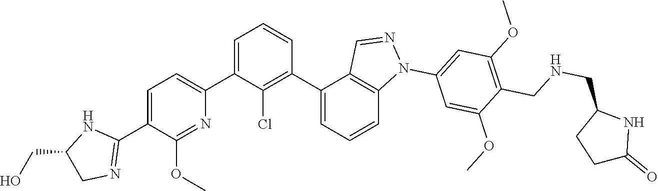 [SMILES: COc1cc(-n2ncc3c(-c4cccc(-c5ccc(C6=NC[C@H](CO)N6)c(OC)n5)c4Cl)cccc32)cc(OC)c1CNC[C@@H]1CCC(=O)N1] |
| | (S)-5-(((4-(4-(2-chloro-3-(5-((R)-5-(hydroxymethyl)-4,5-dihydro-1H- |
| | imidazol-2-yl)-6-methoxypyridin-2-yl)phenyl)-1H-indazol-1-yl)-2,6- |
| | dimethoxybenzyl)amino)methyl)pyrrolidin-2-one |
| 116 | 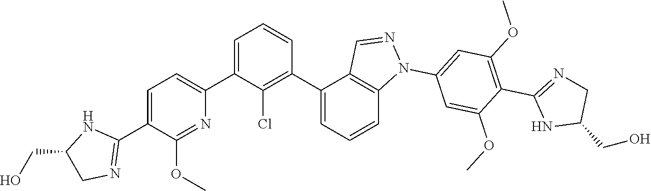 [SMILES: COc1cc(-n2ncc3c(-c4cccc(-c5ccc(C6=NC[C@H](CO)N6)c(OC)n5)c4Cl)cccc32)cc(OC)c1C1=NC[C@H](CO)N1] |
| | ((R)-2-(6-(2-chloro-3-(1-(4-((R)-5-(hydroxymethyl)-4,5-dihydro-1H- |
| | imidazol-2-yl)-3,5-dimethoxyphenyl)-1H-indazol-4-yl)phenyl)-2- |
| | methoxypyridin-3-yl)-4,5-dihydro-1H-imidazol-5-yl)methanol |
| 117 | 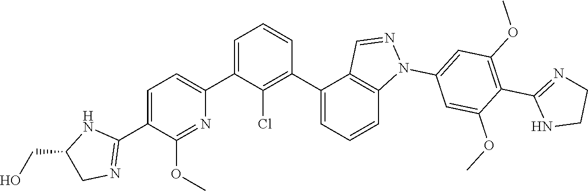 [SMILES: COc1cc(-n2ncc3c(-c4cccc(-c5ccc(C6=NC[C@H](CO)N6)c(OC)n5)c4Cl)cccc32)cc(OC)c1C1=NCCN1] |
| | (R)-(2-(6-(2-chloro-3-(1-(4-(4,5-dihydro-1H-imidazol-2-yl)-3,5- |
| | dimethoxyphenyl)-1H-indazol-4-yl)phenyl)-2-methoxypyridin-3-yl)- |
| | 4,5-dihydro-1H-imidazol-5-yl)methanol |
| 118 | 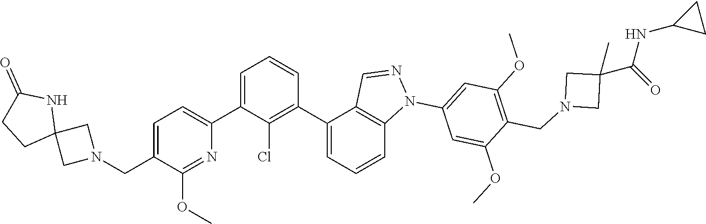 [SMILES: COc1cc(-n2ncc3c(-c4cccc(-c5ccc(CN6CC7(CCC(=O)N7)C6)c(OC)n5)c4Cl)cccc32)cc(OC)c1CN1CC(C)(C(=O)NC2CC2)C1] |
| | 1-(4-(4-(2-chloro-3-(6-methoxy-5-((6-oxo-2,5-diazaspiro[3.4]octan-2- |
| | yl)methyl)pyridin-2-yl)phenyl)-1H-indazol-1-yl)-2,6- |
| | dimethoxybenzyl)-N-cyclopropyl-3-methylazetidine-3-carboxamide |
| 119 | 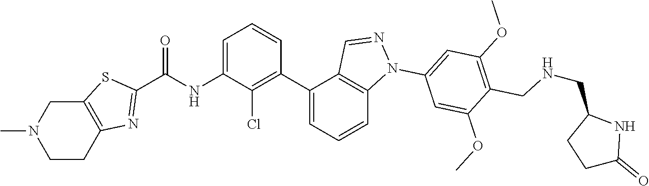 [SMILES: COc1cc(-n2ncc3c(-c4cccc(NC(=O)c5nc6c(s5)CN(C)CC6)c4Cl)cccc32)cc(OC)c1CNC[C@@H]1CCC(=O)N1] |
| | (S)-N-(2-chloro-3-(1-(3,5-dimethoxy-4-((((5-oxopyrrolidin-2- |
| | yl)methyl)amino)methyl)phenyl)-1H-indazol-4-yl)phenyl)-5-methyl- |
| | 4,5,6,7-tetrahydrothiazolo[5,4-c]pyridine-2-carboxamide |
| 120 | 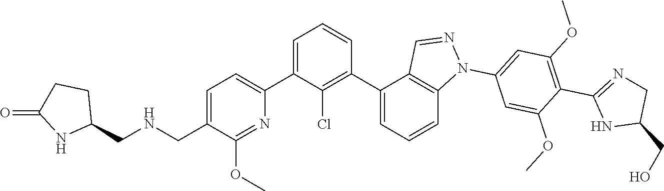 [SMILES: COc1cc(-n2ncc3c(-c4cccc(-c5ccc(CNC[C@@H]6CCC(=O)N6)c(OC)n5)c4Cl)cccc32)cc(OC)c1C1=NC[C@@H](CO)N1] |
| | (S)-5-((((6-(2-chloro-3-(1-(4-((S)-5-(hydroxymethyl)-4,5-dihydro-1H- |
| | imidazol-2-yl)-3,5-dimethoxyphenyl)-1H-indol-4-yl)phenyl)-2- |
| | methoxypyridin-3-yl)methyl)amino)methyl)pyrrolidin-2-one |
| 121 | 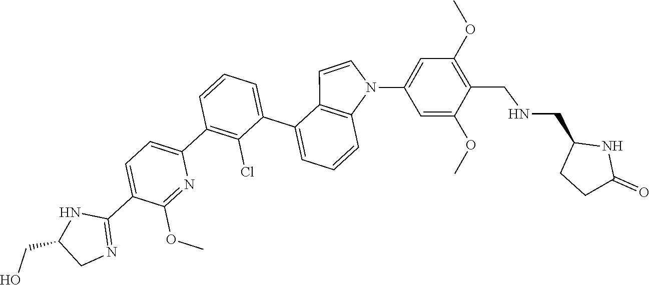 [SMILES: COc1cc(-n2ccc3c(-c4cccc(-c5ccc(C6=NC[C@H](CO)N6)c(OC)n5)c4Cl)cccc32)cc(OC)c1CNC[C@@H]1CCC(=O)N1] |
| | (S)-5-(((4-(4-(2-chloro-3-(5-((R)-5-(hydroxymethyl)-4,5-dihydro-1H- |
| | imidazol-2-yl)-6-methoxypyridin-2-yl)phenyl)-1H-indol-1-yl)-2,6- |
| | dimethoxybenzyl)amino)methyl)pyrrolidin-2-one |
| 122 | 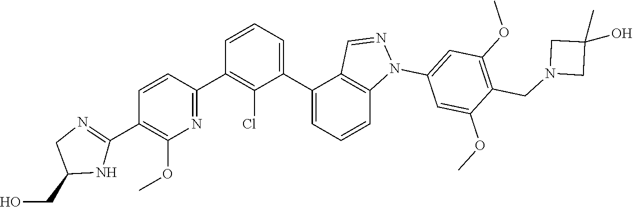 [SMILES: COc1cc(-n2ncc3c(-c4cccc(-c5ccc(C6=NC[C@H](CO)N6)c(OC)n5)c4Cl)cccc32)cc(OC)c1CN1CC(C)(O)C1] |
| | (R)-1-(4-(4-(2-chloro-3-(5-(5-(hydroxymethyl)-4,5-dihydro-1H- |
| | imidazol-2-yl)-6-methoxypyridin-2-yl)phenyl)-1H-indazol-1-yl)-2,6- |
| | dimethoxybenzyl)-3-methylazetidin-3-ol |
| 123 | 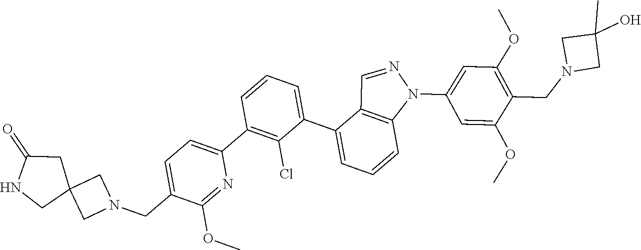 [SMILES: COc1cc(-n2ncc3c(-c4cccc(-c5ccc(CN6CC7(CNC(=O)C7)C6)c(OC)n5)c4Cl)cccc32)cc(OC)c1CN1CC(C)(O)C1] |
| | 2-((6-(2-chloro-3-(1-(4-((3-hydroxy-3-methylazetidin-1-yl)methyl)- |
| | 3,5-dimethoxyphenyl)-1H-indazol-4-yl)phenyl)-2-methoxypyridin-3- |
| | yl)methyl)-2,6-diazaspiro[3.4]octan-7-one |
| 124 | 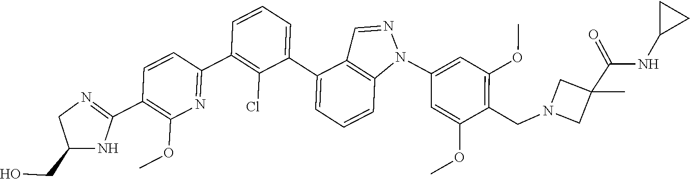 [SMILES: COc1cc(-n2ncc3c(-c4cccc(-c5ccc(C6=NC[C@H](CO)N6)c(OC)n5)c4Cl)cccc32)cc(OC)c1CN1CC(C)(C(=O)NC2CC2)C1] |
| | (R)-1-(4-(4-(2-chloro-3-(5-(5-(hydroxymethyl)-4,5-dihydro-1H- |
| | imidazol-2-yl)-6-methoxypyridin-2-yl)phenyl)-1H-indazol-1-yl)-2,6- |
| | dimethoxybenzyl)-N-cyclopropyl-3-methylazetidine-3-carboxamide |
| 125 | 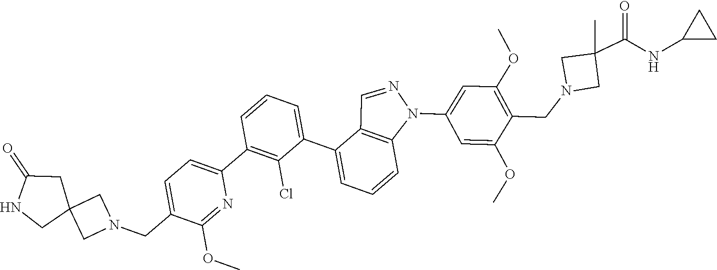 [SMILES: COc1cc(-n2ncc3c(-c4cccc(-c5ccc(CN6CC7(CNC(=O)C7)C6)c(OC)n5)c4Cl)cccc32)cc(OC)c1CN1CC(C)(C(=O)NC2CC2)C1] |
| | 1-(4-(4-(2-chloro-3-(6-methoxy-5-((7-oxo-2,6-diazaspiro[3.4]octan-2- |
| | yl)methyl)pyridin-2-yl)phenyl)-1H-indazol-1-yl)-2,6- |
| | dimethoxybenzyl)-N-cyclopropyl-3-methylazetidine-3-carboxamide |
| 126 | 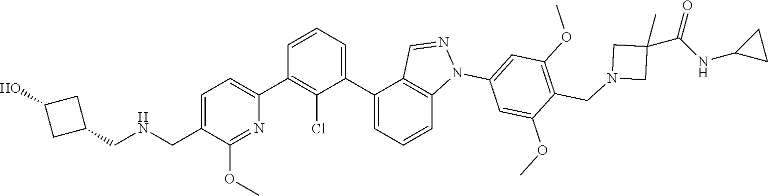 [SMILES: COc1cc(-n2ncc3c(-c4cccc(-c5ccc(CNC[C@H]6C[C@@H](O)C6)c(OC)n5)c4Cl)cccc32)cc(OC)c1CN1CC(C)(C(=O)NC2CC2)C1] |
| | 1-(4-(4-(2-chloro-3-(5-((((cis-3- |
| | hydroxycyclobutyl)methyl)amino)methyl)-6-methoxypyridin-2- |
| | yl)phenyl)-1H-indazol-1-yl)-2,6-dimethoxybenzyl)-N-cyclopropyl-3- |
| | methylazetidine-3-carboxamide |
| 127 | 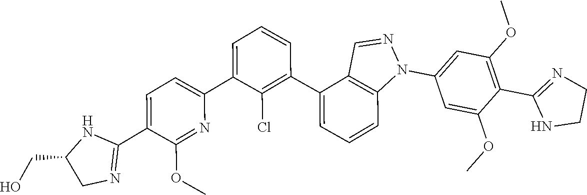 [SMILES: COc1cc(-n2ncc3c(-c4cccc(-c5ccc(C6=NC[C@H](CO)N6)c(OC)n5)c4Cl)cccc32)cc(OC)c1C1=NCCN1] |
| | (R)-(2-(6-(2-chloro-3-(1-(4-(4,5-dihydro-1H-imidazol-2-yl)-3,5- |
| | dimethoxyphenyl)-1H-indazol-4-yl)phenyl)-2-methoxypyridin-3-yl)- |
| | 4,5-dihydro-1H-imidazol-5-yl)methanol |
| 128 | 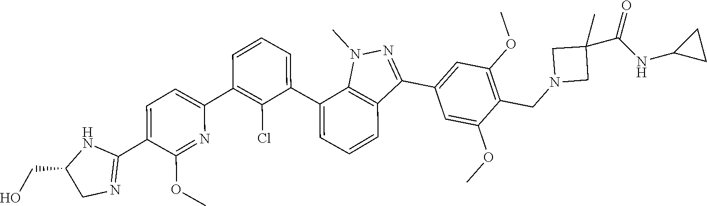 [SMILES: COc1cc(-c2nn(C)c3c(-c4cccc(-c5ccc(C6=NC[C@H](CO)N6)c(OC)n5)c4Cl)cccc23)cc(OC)c1CN1CC(C)(C(=O)NC2CC2)C1] |
| | (R)-1-(4-(7-(2-chloro-3-(5-(5-(hydroxymethyl)-4,5-dihydro-1H- |
| | imidazol-2-yl)-6-methoxypyridin-2-yl)phenyl)-1-methyl-1H-indazol- |
| | 3-yl)-2,6-dimethoxybenzyl)-N-cyclopropyl-3-methylazetidine-3- |
| | carboxamide |
| 129 | 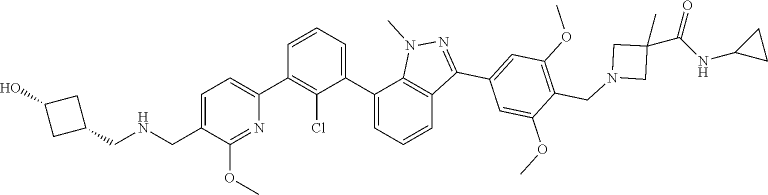 [SMILES: COc1cc(-c2nn(C)c3c(-c4cccc(-c5ccc(CNC[C@H]6C[C@@H](O)C6)c(OC)n5)c4Cl)cccc23)cc(OC)c1CN1CC(C)(C(=O)NC2CC2)C1] |
| | 1-(4-(7-(2-chloro-3-(5-((((cis-3- |
| | hydroxycyclobutyl)methyl)amino)methyl)-6-methoxypyridin-2- |
| | yl)phenyl)-1-methyl-1H-indazol-3-yl)-2,6-dimethoxybenzyl)-N- |
| | cyclopropyl-3-methylazetidine-3-carboxamide |
| 130 | 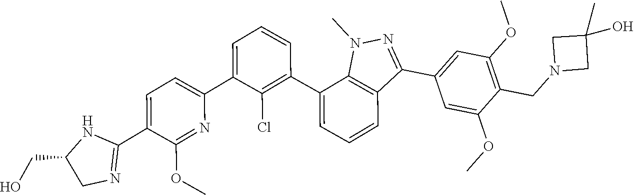 [SMILES: COc1cc(-c2nn(C)c3c(-c4cccc(-c5ccc(C6=NC[C@H](CO)N6)c(OC)n5)c4Cl)cccc23)cc(OC)c1CN1CC(C)(O)C1] |
| | (R)-1-(4-(7-(2-chloro-3-(5-(5-(hydroxymethyl)-4,5-dihydro-1H- |
| | imidazol-2-yl)-6-methoxypyridin-2-yl)phenyl)-1-methyl-1H-indazol- |
| | 3-yl)-2,6-dimethoxybenzyl)-3-methylazetidin-3-ol |
| 131 | 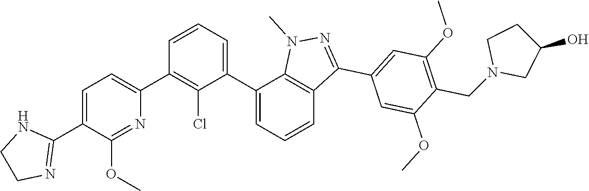 [SMILES: COc1cc(-c2nn(C)c3c(-c4cccc(-c5ccc(C6=NCCN6)c(OC)n5)c4Cl)cccc23)cc(OC)c1CN1CC[C@@H](O)C1] |
| | (R)-1-(4-(7-(2-chloro-3-(5-(4,5-dihydro-1H-imidazol-2-yl)-6- |
| | methoxypyridin-2-yl)phenyl)-1-methyl-1H-indazol-3-yl)-2,6- |
| | dimethoxybenzyl)pyrrolidin-3-ol |
| 132 | 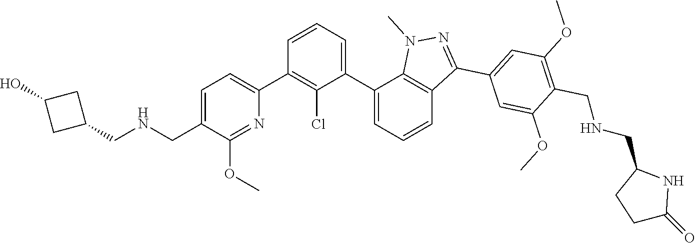 [SMILES: COc1cc(-c2nn(C)c3c(-c4cccc(-c5ccc(CNC[C@H]6C[C@@H](O)C6)c(OC)n5)c4Cl)cccc23)cc(OC)c1CNC[C@@H]1CCC(=O)N1] |
| | (S)-5-(((4-(7-(2-chloro-3-(5-((((cis-3- |
| | hydroxycyclobutyl)methyl)amino)methyl)-6-methoxypyridin-2- |
| | yl)phenyl)-1-methyl-1H-indazol-3-yl)-2,6- |
| | dimethoxybenzyl)amino)methyl)pyrrolidin-2-one |
| 133 | 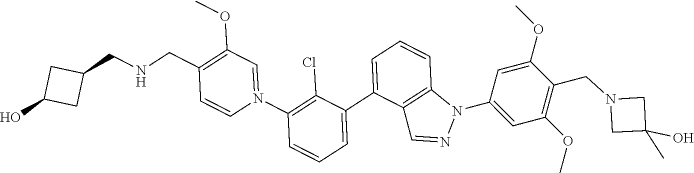 |
| | 1-(4-(4-(2-chloro-3-(5-((((cis-3- |
| | hydroxycyclobutyl)methyl)amino)methyl)-6-methoxypyridin-2- |
| | yl)phenyl)-1H-indazol-1-yl)-2,6-dimethoxybenzyl)-3-methylazetidin- |
| | 3-o1 |
| 134 | 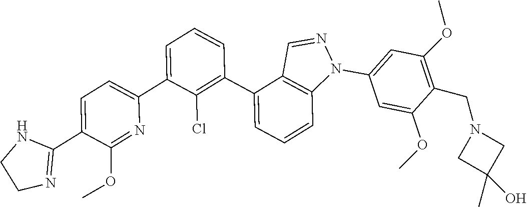 [SMILES: COc1cc(-n2ncc3c(-c4cccc(-c5ccc(C6=NCCN6)c(OC)n5)c4Cl)cccc32)cc(OC)c1CN1CC(C)(O)C1] |
| | 1-(4-(4-(2-chloro-3-(5-(4,5-dihydro-1H-imidazol-2-yl)-6- |
| | methoxypyridin-2-yl)phenyl)-1H-indazol-1-yl)-2,6- |
| | dimethoxybenzyl)-3-methylazetidin-3-ol |
| 135 | 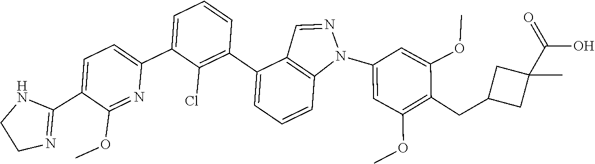 [SMILES: COc1cc(-n2ncc3c(-c4cccc(-c5ccc(C6=NCCN6)c(OC)n5)c4Cl)cccc32)cc(OC)c1CC1CC(C)(C(=O)O)C1] |
| | 1-(4-(4-(2-chloro-3-(5-(4,5-dihydro-1H-imidazol-2-yl)-6- |
| | methoxypyridin-2-yl)phenyl)-1H-indazol-1-yl)-2,6- |
| | dimethoxybenzyl)-3-methylazetidine-3-carboxylic acid |
| 136 | 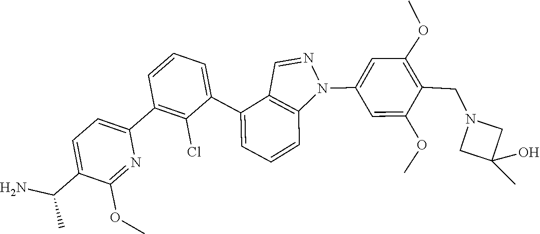 [SMILES: COc1cc(-n2ncc3c(-c4cccc(-c5ccc([C@H](C)N)c(OC)n5)c4Cl)cccc32)cc(OC)c1CN1CC(C)(O)C1] |
| | (S)-1-(4-(4-(3-(5-(1-aminoethyl)-6-methoxypyridin-2-yl)-2- |
| | chlorophenyl)-1H-indazol-1-yl)-2,6-dimethoxybenzyl)-3- |
| | methylazetidin-3-ol |
| 137 | 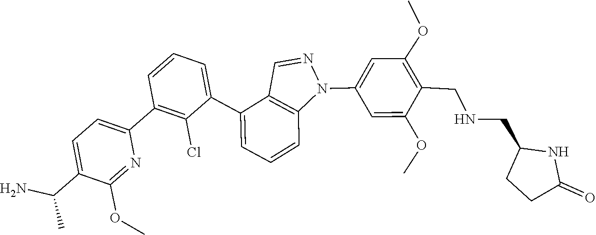 [SMILES: COc1cc(-n2ncc3c(-c4cccc(-c5ccc([C@H](C)N)c(OC)n5)c4Cl)cccc32)cc(OC)c1CNC[C@@H]1CCC(=O)N1] |
| | (S)-5-(((4-(7-(2-chloro-3-(5-(4,5-dihydro-1H-imidazol-2-yl)-6- |
| | methoxypyridin-2-yl)phenyl)-1-methyl-1H-indazol-3-yl)-2,6- |
| | dimethoxybenzyl)amino)methyl)pyrrolidin-2-one |
| 138 | 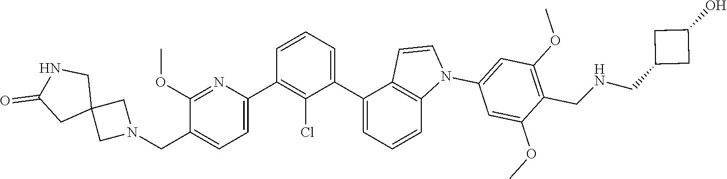 [SMILES: COc1cc(-n2ccc3c(-c4cccc(-c5ccc(CN6CC7(CNC(=O)C7)C6)c(OC)n5)c4Cl)cccc32)cc(OC)c1CNC[C@H]1C[C@@H](O)C1] |
| | 2-((6-(2-chloro-3-(1-(4-((((cis-3- |
| | hydroxycyclobutyl)methyl)amino)methyl)-3,5-dimethoxyphenyl)-1H- |
| | indol-4-yl)phenyl)-2-methoxypyridin-3-yl)methyl)-2,6- |
| | diazaspiro[3.4]octan-7-one |
| 139 | 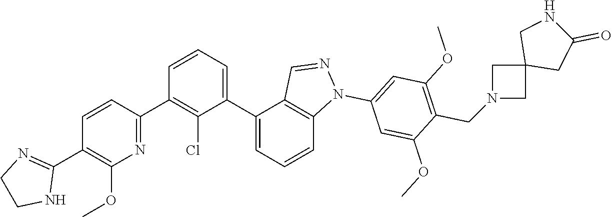 [SMILES: COc1cc(-n2ncc3c(-c4cccc(-c5ccc(C6=NCCN6)c(OC)n5)c4Cl)cccc32)cc(OC)c1CN1CC2(CNC(=O)C2)C1] |
| | 2-(4-(4-(2-chloro-3-(5-(4,5-dihydro-1H-imidazol-2-yl)-6- |
| | methoxypyridin-2-yl)phenyl)-1H-indazol-1-yl)-2,6- |
| | dimethoxybenzyl)-2,6-diazaspiro[3.4]octan-7-one |
| 140 | 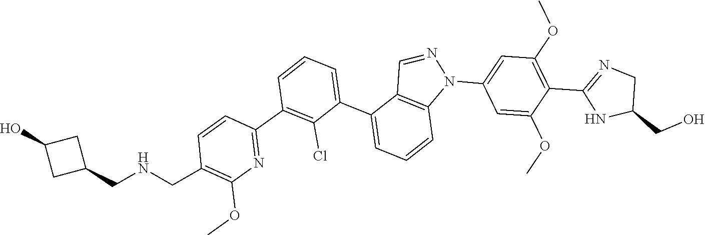 [SMILES: COc1cc(-n2ncc3c(-c4cccc(-c5ccc(CNC[C@H]6C[C@@H](O)C6)c(OC)n5)c4Cl)cccc32)cc(OC)c1C1=NC[C@@H](CO)N1] |
| | cis-3-((((6-(2-chloro-3-(1-(4-((S)-5-(hydroxymethyl)-4,5-dihydro-1H- |
| | imidazol-2-yl)-3,5-dimethoxyphenyl)-1H-indazol-4-yl)phenyl)-2- |
| | methoxypyridin-3-yl)methyl)amino)methyl)cyclobutan-1-ol |
| 141 | 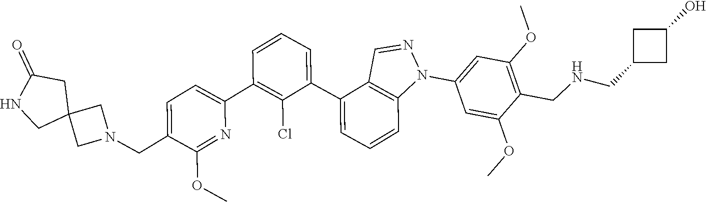 [SMILES: COc1cc(-n2ncc3c(-c4cccc(-c5ccc(CN6CC7(CNC(=O)C7)C6)c(OC)n5)c4Cl)cccc32)cc(OC)c1CNC[C@H]1C[C@@H](O)C1] |
| | 2-((6-(2-chloro-3-(1-(4-((((cis-3- |
| | hydroxycyclobutyl)methyl)amino)methyl)-3,5-dimethoxyphenyl)-1H- |
| | indazol-4-yl)phenyl)-2-methoxypyridin-3-yl)methyl)-2,6- |
| | diazaspiro[3.4]octan-7-one |
| 142 | 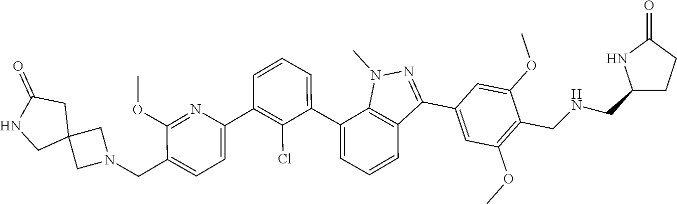 [SMILES: COc1cc(-c2nn(C)c3c(-c4cccc(-c5ccc(CN6CC7(CNC(=O)C7)C6)c(OC)n5)c4Cl)cccc23)cc(OC)c1CNC[C@@H]1CCC(=O)N1] |
| | (S)-2-((6-(2-chloro-3-(3-(3,5-dimethoxy-4-((((5-oxopyrrolidin-2- |
| | yl)methyl)amino)methyl)phenyl)-1-methyl-1H-indazol-7-yl)phenyl)- |
| | 2-methoxypyridin-3-yl)methyl)-2,6-diazaspiro[3.4]octan-7-one |
| 143 | 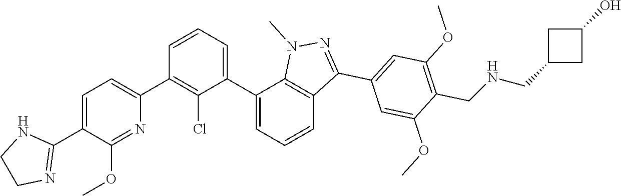 [SMILES: COc1cc(-c2nn(C)c3c(-c4cccc(-c5ccc(C6=NCCN6)c(OC)n5)c4Cl)cccc23)cc(OC)c1CNC[C@H]1C[C@@H](O)C1] |
| | cis-3-(((4-(7-(2-chloro-3-(5-(4,5-dihydro-1H-imidazol-2-yl)-6- |
| | methoxypyridin-2-yl)phenyl)-1-methyl-1H-indazol-3-yl)-2,6- |
| | dimethoxybenzyl)amino)methyl)cyclobutan-1-ol |
| 144 | 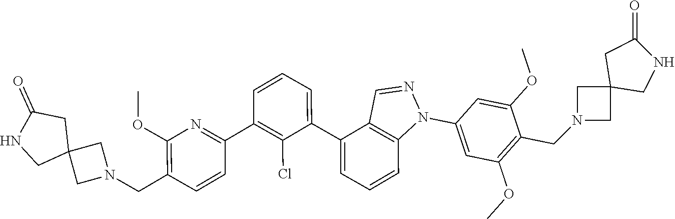 [SMILES: COc1cc(-n2ncc3c(-c4cccc(-c5ccc(CN6CC7(CNC(=O)C7)C6)c(OC)n5)c4Cl)cccc32)cc(OC)c1CN1CC2(CNC(=O)C2)C1] |
| | 2-(4-(4-(2-chloro-3-(6-methoxy-5-((7-oxo-2,6-diazaspiro[3.4]octan-2- |
| | yl)methyl)pyridin-2-yl)phenyl)-1H-indazol-1-yl)-2,6- |
| | dimethoxybenzyl)-2,6-diazaspiro[3.4]octan-7-one |
| 145 | 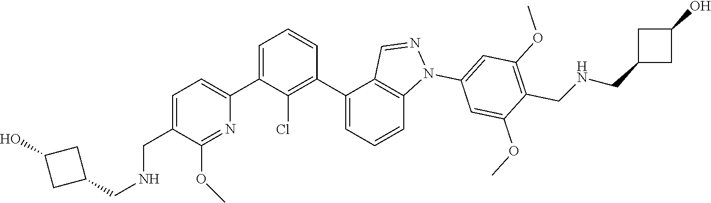 [SMILES: COc1cc(-n2ncc3c(-c4cccc(-c5ccc(CNC[C@H]6C[C@@H](O)C6)c(OC)n5)c4Cl)cccc32)cc(OC)c1CNC[C@H]1C[C@@H](O)C1] |
| | cis-3-((((6-(2-chloro-3-(1-(4-((((cis-3- |
| | hydroxycyclobutyl)methyl)amino)methyl)-3,5-dimethoxyphenyl)-1H- |
| | indazol-4-yl)phenyl)-2-methoxypyridin-3- |
| | yl)methyl)amino)methyl)cyclobutan-1-ol |
| 146 | 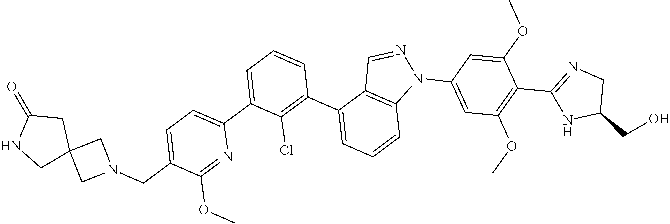 [SMILES: COc1cc(-n2ncc3c(-c4cccc(-c5ccc(CN6CC7(CNC(=O)C7)C6)c(OC)n5)c4Cl)cccc32)cc(OC)c1C1=NC[C@@H](CO)N1] |
| | (S)-2-((6-(2-chloro-3-(1-(4-(5-(hydroxymethyl)-4,5-dihydro-1H- |
| | imidazol-2-yl)-3,5-dimethoxyphenyl)-1H-indazol-4-yl)phenyl)-2- |
| | methoxypyridin-3-yl)methyl)-2,6-diazaspiro[3.4]octan-7-one |
| 147 | 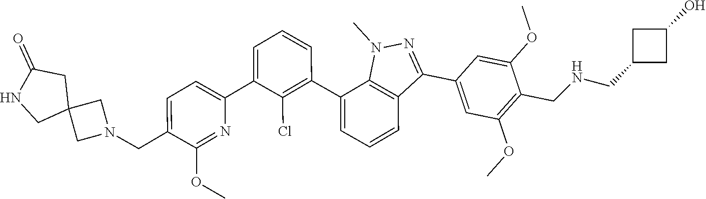 [SMILES: COc1cc(-c2nn(C)c3c(-c4cccc(-c5ccc(CN6CC7(CNC(=O)C7)C6)c(OC)n5)c4Cl)cccc23)cc(OC)c1CNC[C@H]1C[C@@H](O)C1] |
| | 2-((6-(2-chloro-3-(3-(4-((((cis-3- |
| | hydroxycyclobutyl)methyl)amino)methyl)-3,5-dimethoxyphenyl)-1- |
| | methyl-1H-indazol-7-yl)phenyl)-2-methoxypyridin-3-yl)methyl)-2,6- |
| | diazaspiro[3.4]octan-7-one |
| 148 | 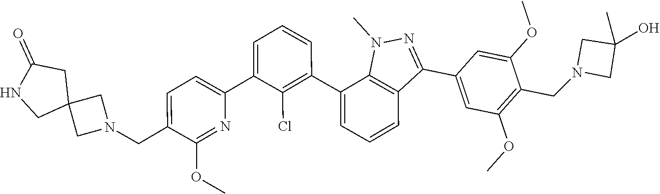 [SMILES: COc1cc(-c2nn(C)c3c(-c4cccc(-c5ccc(CN6CC7(CNC(=O)C7)C6)c(OC)n5)c4Cl)cccc23)cc(OC)c1CN1CC(C)(O)C1] |
| | 2-((6-(2-chloro-3-(3-(4-((3-hydroxy-3-methylazetidin-1-yl)methyl)- |
| | 3,5-dimethoxyphenyl)-1-methyl-1H-indazol-7-yl)phenyl)-2- |
| | methoxypyridin-3-yl)methyl)-2,6-diazaspiro[3.4]octan-7-one |
| 149 | 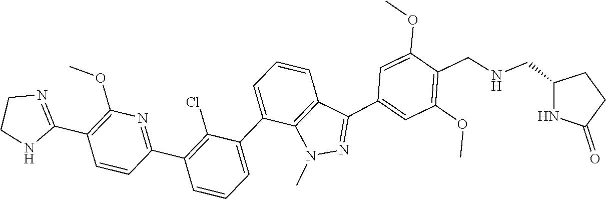 [SMILES: COc1cc(-c2nn(C)c3c(-c4cccc(-c5ccc(C6=NCCN6)c(OC)n5)c4Cl)cccc23)cc(OC)c1CNC[C@@H]1CCC(=O)N1] |
| | (S)-5-(((4-(7-(2-chloro-3-(5-(4,5-dihydro-1H-imidazol-2-yl)-6- |
| | methoxypyridin-2-yl)phenyl)-1-methyl-1H-indazol-3-yl)-2,6- |
| | dimethoxybenzyl)amino)methyl)pyrrolidin-2-one |
| 150 | 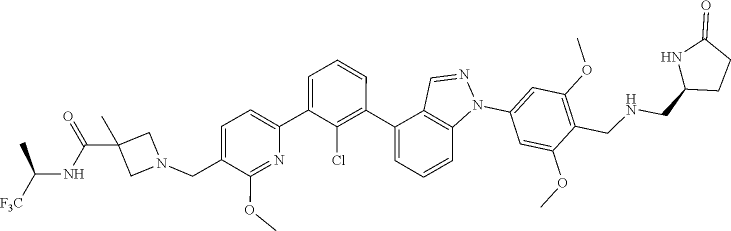 [SMILES: COc1cc(-n2ncc3c(-c4cccc(-c5ccc(CN6CC(C)(C(=O)N[C@H](C)C(F)(F)F)C6)c(OC)n5)c4Cl)cccc32)cc(OC)c1CNC[C@@H]1CCC(=O)N1] |
| | 1-((6-(2-chloro-3-(1-(3,5-dimethoxy-4-(((((S)-5-oxopyrrolidin~2- |
| | yl)methyl)amino)methyl)phenyl)-1H-indazol-4-yl)phenyl)-2- |
| | methoxypyridin-3-yl)methyl)-3-methyl-N-((R)-1,1,1-trifluoropropan- |
| | 2-yl)azetidine-3-carboxamide |
| 151 | 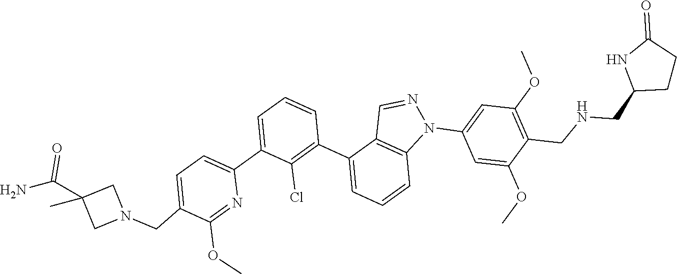 [SMILES: COc1cc(-n2ncc3c(-c4cccc(-c5ccc(CN6CC(C)(C(N)=O)C6)c(OC)n5)c4Cl)cccc32)cc(OC)c1CNC[C@@H]1CCC(=O)N1] |
| | (S)-1-((6-(2-chloro-3-(1-(3,5-dimethoxy-4-((((5-oxopyrrolidin-2- |
| | yl)methyl)amino)methyl)phenyl)-1H-indazol-4-yl)phenyl)-2- |
| | methoxypyridin-3-yl)methyl)-3-methylazetidine-3-carboxamide |
| 152 | 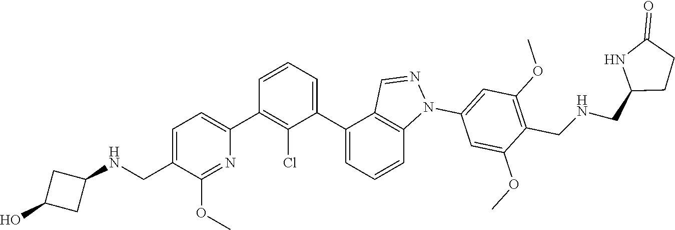 [SMILES: COc1cc(-n2ncc3c(-c4cccc(-c5ccc(CN[C@H]6C[C@@H](O)C6)c(OC)n5)c4Cl)cccc32)cc(OC)c1CNC[C@@H]1CCC(=O)N1] |
| | (S)-5-(((4-(4-(2-chloro-3-(5-(((cis-3- |
| | hydroxycyclobutyl)amino)methyl)-6-methoxypyridin-2-yl)phenyl)- |
| | 1H-indazol-1-yl)-2,6-dimethoxybenzyl)amino)methyl)pyrrolidin-2- |
| | one |
| 153 | 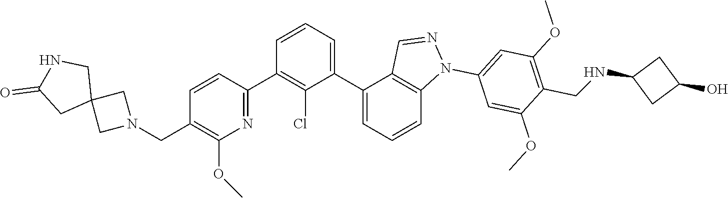 [SMILES: COc1cc(-n2ncc3c(-c4cccc(-c5ccc(CN6CC7(CNC(=O)C7)C6)c(OC)n5)c4Cl)cccc32)cc(OC)c1CN[C@H]1C[C@@H](O)C1] |
| | 2-((6-(2-chloro-3-(1-(4-(((cis-3-hydroxycyclobutyl)amino)methyl)- |
| | 3,5-dimethoxyphenyl)-1H-indazol-4-yl)phenyl)-2-methoxypyridin-3- |
| | yl)methyl)-2,6-diazaspiro[3.4]octan-7-one |
| 154 | 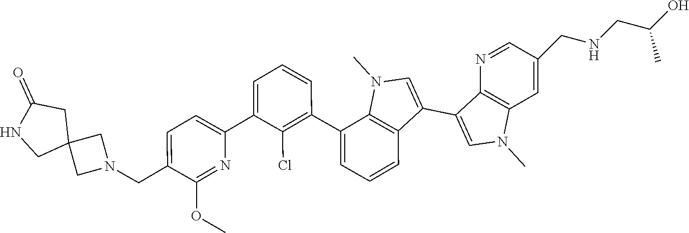 [SMILES: COc1nc(-c2cccc(-c3cccc4c(-c5cn(C)c6cc(CNC[C@@H](C)O)cnc56)cn(C)c34)c2Cl)ccc1CN1CC2(CNC(=O)C2)C1] |
| | (R)-2-((6-(2-chloro-3-(3-(6-(((2-hydroxypropyl)amino)methyl)-1- |
| | methyl-1H-pyrrolo[3,2-b]pyridin-3-yl)-1-methyl-1H-indol-7- |
| | yl)phenyl)-2-methoxypyridin-3-yl)methyl)-2,6-diazaspiro[3.4]octan- |
| | 7-one |
| 155 | 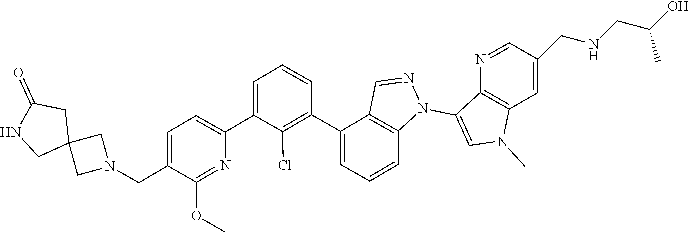 [SMILES: COc1nc(-c2cccc(-c3cccc4c3cnn4-c3cn(C)c4cc(CNC[C@@H](C)O)cnc34)c2Cl)ccc1CN1CC2(CNC(=O)C2)C1] |
| | (R)-2-((6-(2-chloro-3-(1-(6-(((2-hydroxypropyl)amino)methyl)-1- |
| | methyl-1H-pyrrolo[3,2-b]pyridin-3-yl)-1H-indazol-4-yl)phenyl)-2- |
| | methoxypyridin-3-yl)methyl)-2,6-diazaspiro[3.4]octan-7-one |
| 156 | 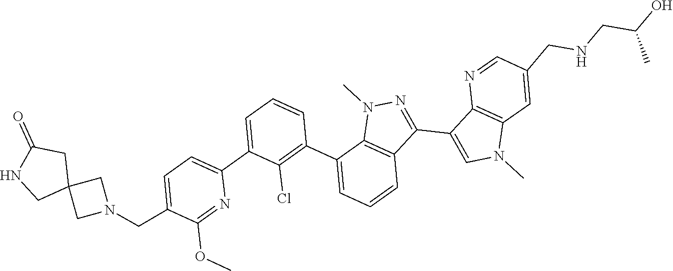 [SMILES: COc1nc(-c2cccc(-c3cccc4c(-c5cn(C)c6cc(CNC[C@@H](C)O)cnc56)nn(C)c34)c2Cl)ccc1CN1CC2(CNC(=O)C2)C1] |
| | (R)-2-((6-(2-chloro-3-(3-(6-(((2-hydroxypropyl)amino)methyl)-1- |
| | methyl-1H-pyrrolo[3,2-b]pyridin-3-yl)-1-methyl-1H-indazol-7- |
| | yl)phenyl)-2-methoxypyridin-3-yl)methyl)-2,6-diazaspiro[3.4]octan- |
| | 7-one |
| 157 | 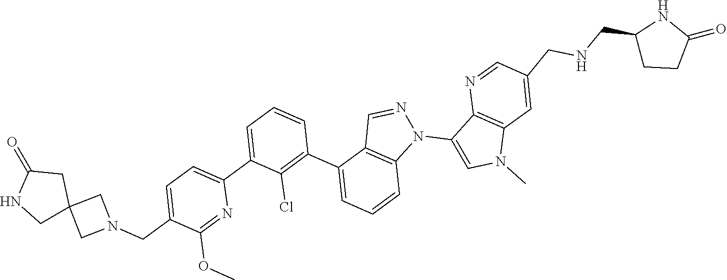 [SMILES: COc1nc(-c2cccc(-c3cccc4c3cnn4-c3cn(C)c4cc(CNC[C@@H]5CCC(=O)N5)cnc34)c2Cl)ccc1CN1CC2(CNC(=O)C2)C1] |
| | (S)-2-((6-(2-chloro-3-(1-(1-methyl-6-((((5-oxopyrrolidin-2- |
| | yl)methyl)amino)methyl)-1H-pyrrolo[3,2-b]pyridin-3-yl)-1H-indazol- |
| | 4-yl)phenyl)-2-methoxypyridin-3-yl)methyl)-2,6- |
| | diazaspiro[3.4]octan-7-one |
| 158 | 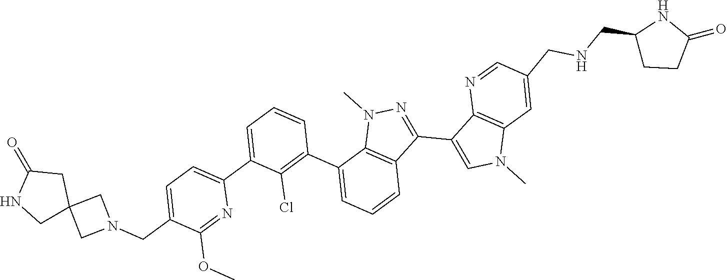 [SMILES: COc1nc(-c2cccc(-c3cccc4c(-c5cn(C)c6cc(CNC[C@@H]7CCC(=O)N7)cnc56)nn(C)c34)c2Cl)ccc1CN1CC2(CNC(=O)C2)C1] |
| | (S)-2-((6-(2-chloro-3-(1-methyl-3-(1-methyl-6-((((5-oxopyrrolidin-2- |
| | yl)methyl)amino)methyl)-1H-pyrrolo[3,2-b]pyridin-3-yl)-1H-indazol- |
| | 7-yl)phenyl)-2-methoxypyridin-3-yl)methyl)-2,6- |
| | diazaspiro[3.4]octan-7-one |
| 159 | 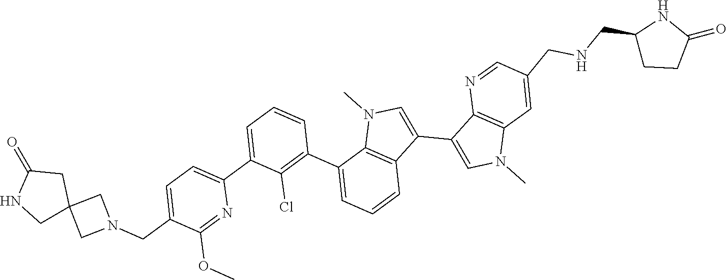 [SMILES: COc1nc(-c2cccc(-c3cccc4c(-c5cn(C)c6cc(CNC[C@@H]7CCC(=O)N7)cnc56)cn(C)c34)c2Cl)ccc1CN1CC2(CNC(=O)C2)C1] |
| | (S)-2-((6-(2-chloro-3-(1-methyl-3-(1-methyl-6-((((5-oxopyrrolidin-2- |
| | yl)methyl)amino)methyl)-1H-pyrrolo[3,2-b]pyridin-3-yl)-1H-indol-7- |
| | yl)phenyl)-2-methoxypyridin-3-yl)methyl)-2,6-diazaspiro[3.4]octan- |
| | 7-one |
| 160 | 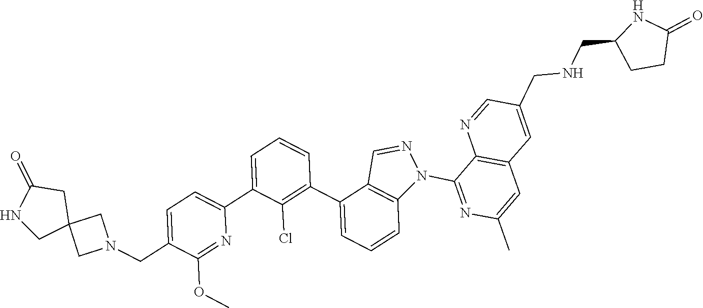 [SMILES: COc1nc(-c2cccc(-c3cccc4c3cnn4-c3nc(C)cc4cc(CNC[C@@H]5CCC(=O)N5)cnc34)c2Cl)ccc1CN1CC2(CNC(=O)C2)C1] |
| | (S)-2-((6-(2-chloro-3-(1-(6-methyl-3-((((5-oxopyrrolidin-2- |
| | yl)methyl)amino)methyl)-1,7-naphthyridin-8-yl)-1H-indazol-4- |
| | yl)phenyl)-2-methoxypyridin-3-yl)methyl)-2,6-diazaspiro[3.4]octan- |
| | 7-one |
| 161 | 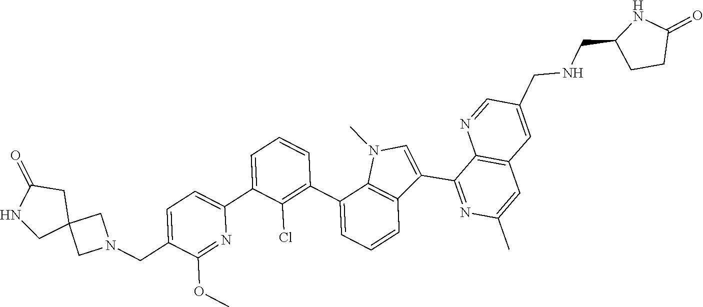 [SMILES: COc1nc(-c2cccc(-c3cccc4c(-c5nc(C)cc6cc(CNC[C@@H]7CCC(=O)N7)cnc56)cn(C)c34)c2Cl)ccc1CN1CC2(CNC(=O)C2)C1] |
| | (S)-2-((6-(2-chloro-3-(1-methyl-3-(6-methyl-3-((((5-oxopyrrolidin-2- |
| | yl)methyl)amino)methyl)-1,7-naphthyridin-8-yl)-1H-indol-7- |
| | yl)phenyl)-2-methoxypyridin-3-yl)methyl)-2,6-diazaspiro[3.4]octan- |
| | 7-one |
| 162 | 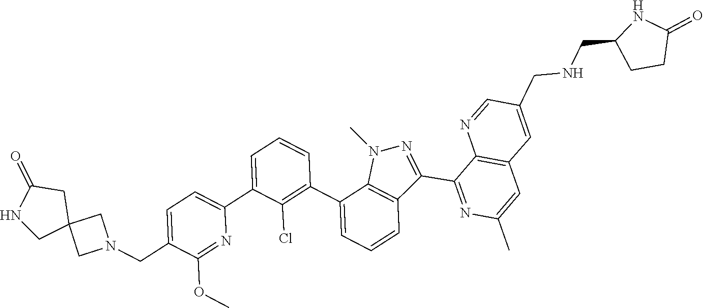 [SMILES: COc1nc(-c2cccc(-c3cccc4c(-c5nc(C)cc6cc(CNC[C@@H]7CCC(=O)N7)cnc56)nn(C)c34)c2Cl)ccc1CN1CC2(CNC(=O)C2)C1] |
| | (S)-2-((6-(2-chloro-3-(1-methyl-3-(6-methyl-3-((((5-oxopyrrolidin-2- |
| | yl)methyl)amino)methyl)-1,7-naphthyridin-8-yl)-1H-indazol-7- |
| | yl)phenyl)-2-methoxypyridin-3-yl)methyl)-2,6-diazaspiro[3.4]octan- |
| | 7-one |
| 163 | 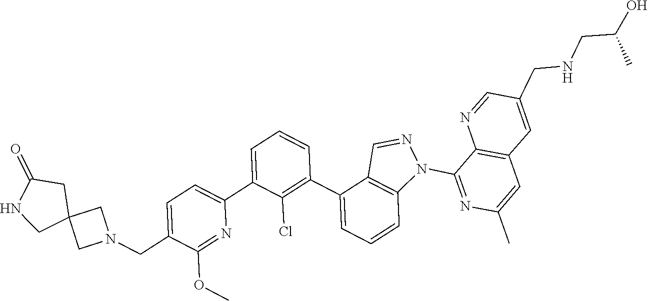 [SMILES: COc1nc(-c2cccc(-c3cccc4c3cnn4-c3nc(C)cc4cc(CNC[C@@H](C)O)cnc34)c2Cl)ccc1CN1CC2(CNC(=O)C2)C1] |
| | (R)-2-((6-(2-chloro-3-(1-(3-(((2-hydroxypropyl)amino)methyl)-6- |
| | methyl-1,7-naphthyridin-8-yl)-1H-indazol-4-yl)phenyl)-2- |
| | methoxypyridin-3-yl)methyl)-2,6-diazaspiro[3.4]octan-7-one |
| 164 | 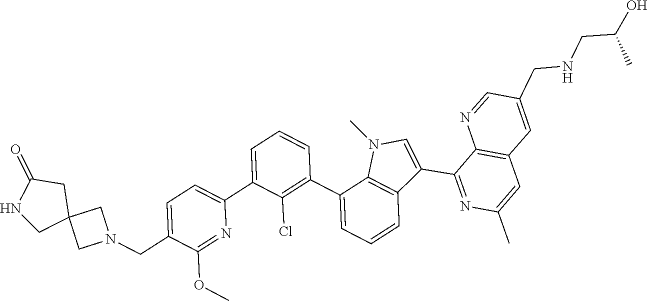 [SMILES: COc1nc(-c2cccc(-c3cccc4c(-c5nc(C)cc6cc(CNC[C@@H](C)O)cnc56)cn(C)c34)c2Cl)ccc1CN1CC2(CNC(=O)C2)C1] |
| | (R)-2-((6-(2-chloro-3-(3-(3-(((2-hydroxypropyl)amino)methyl)-6- |
| | methyl-1,7-naphthyridin-8-yl)-1-methyl-1H-indol-7-yl)phenyl)-2- |
| | methoxypyridin-3-yl)methyl)-2,6-diazaspiro[3.4]octan-7-one |
| 165 | 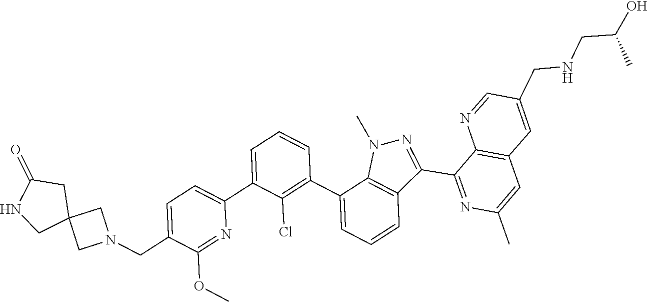 [SMILES: COc1nc(-c2cccc(-c3cccc4c(-c5nc(C)cc6cc(CNC[C@@H](C)O)cnc56)nn(C)c34)c2Cl)ccc1CN1CC2(CNC(=O)C2)C1] |
| | (R)-2-((6-(2-chloro-3-(3-(3-(((2-hydroxypropyl)amino)methyl)-6- |
| | methyl-1,7-naphthyridin-8-yl)-1-methyl-1H-indazol-7-yl)phenyl)-2- |
| | methoxypyridin-3-yl)methyl)-2,6-diazaspiro[3.4]octan-7-one |
| 166 | 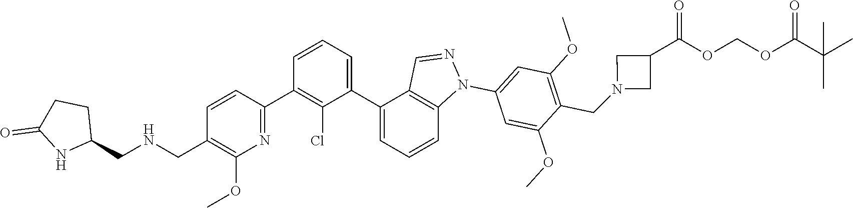 [SMILES: COc1cc(-n2ncc3c(-c4cccc(-c5ccc(CNC[C@@H]6CCC(=O)N6)c(OC)n5)c4Cl)cccc32)cc(OC)c1CN1CC(C(=O)OCOC(=O)C(C)(C)C)C1] |
| | (pivaloyloxy)methyl (S)-1-(4-(4-(2-chloro-3-(6-methoxy-5-((((5- |
| | oxopyrrolidin-2-yl)methyl)amino)methyl)pyridin-2-yl)phenyl)-1H- |
| | indazol-1-yl)-2,6-dimethoxybenzyl)azetidine-3-carboxylate |
| 167 | 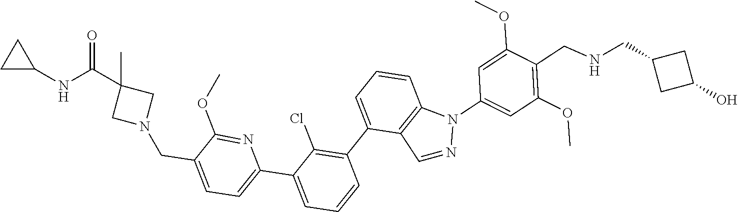 [SMILES: COc1cc(-n2ncc3c(-c4cccc(-c5ccc(CN6CC(C)(C(=O)NC7CC7)C6)c(OC)n5)c4Cl)cccc32)cc(OC)c1CNC[C@H]1C[C@@H](O)C1] |
| | 1-((6-(2-chloro-3-(1-(4-((((cis-3- |
| | hydroxycyclobutyl)methyl)amino)methyl)-3,5-dimethoxyphenyl)-1H- |
| | indazol-4-yl)phenyl)-2-methoxypyridin-3-yl)methyl)-N-cyclopropyl- |
| | 3-methylazetidine-3-carboxamide |
| 168 | 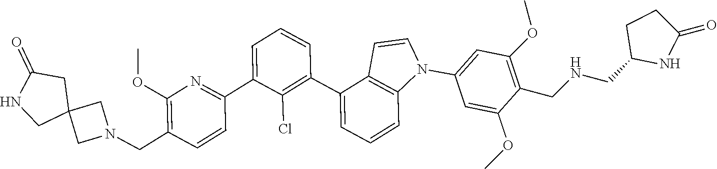 [SMILES: COc1cc(-n2ccc3c(-c4cccc(-c5ccc(CN6CC7(CNC(=O)C7)C6)c(OC)n5)c4Cl)cccc32)cc(OC)c1CNC[C@@H]1CCC(=O)N1] |
| | (S)-2-((6-(2-chloro-3-(1-(3,5-dimethoxy-4-((((5-oxopyrrolidin-2- |
| | yl)methyl)amino)methyl)phenyl)-1H-indol-4-yl)phenyl)-2- |
| | methoxypyridin-3-yl)methyl)-2,6-diazaspiro[3.4]octan-7-one |
| 169 | 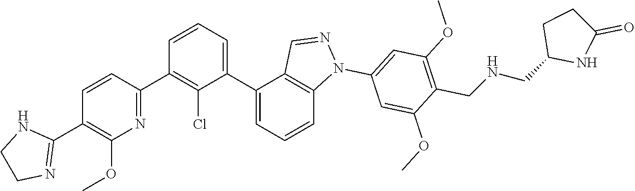 [SMILES: COc1cc(-n2ncc3c(-c4cccc(-c5ccc(C6=NCCN6)c(OC)n5)c4Cl)cccc32)cc(OC)c1CNC[C@@H]1CCC(=O)N1] |
| | (S)-5-(((4-(4-(2-chloro-3-(5-(4,5-dihydro-1H-imidazol-2-yl)-6- |
| | methoxypyridin-2-yl)phenyl)-1H-indol-1-yl)-2,6- |
| | dimethoxybenzyl)amino)methyl)pyrrolidin-2-one |
| 170 | 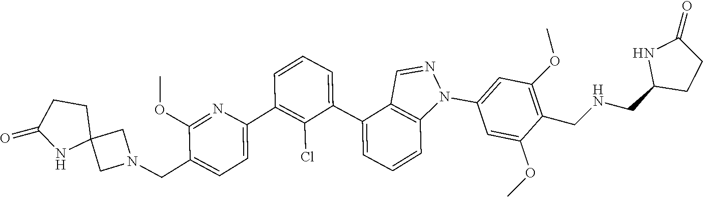 [SMILES: COc1cc(-n2ncc3c(-c4cccc(-c5ccc(CN6CC7(CCC(=O)N7)C6)c(OC)n5)c4Cl)cccc32)cc(OC)c1CNC[C@@H]1CCC(=O)N1] |
| | (S)-2-((6-(2-chloro-3-(1-(3,5-dimethoxy-4-((((5-oxopyrrolidin-2- |
| | yl)methyl)amino)methyl)phenyl)-1H-indazol-4-yl)phenyl)-2- |
| | methoxypyridin-3-yl)methyl)-2,5-diazaspiro[3.4]octan-6-one |
| 171 | 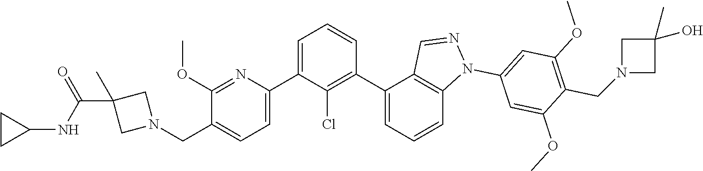 [SMILES: COc1cc(-n2ncc3c(-c4cccc(-c5ccc(CN6CC(C)(C(=O)NC7CC7)C6)c(OC)n5)c4Cl)cccc32)cc(OC)c1CN1CC(C)(O)C1] |
| | 1-((6-(2-chloro-3-(1-(4-((3-hydroxy-3-methylazetidin-1-yl)methyl)- |
| | 3,5-dimethoxyphenyl)-1H-indazol-4-yl)phenyl)-2-methoxypyridin-3- |
| | yl)methyl)-N-cyclopropyl-3-methylazetidine-3-carboxamide |
| 172 | 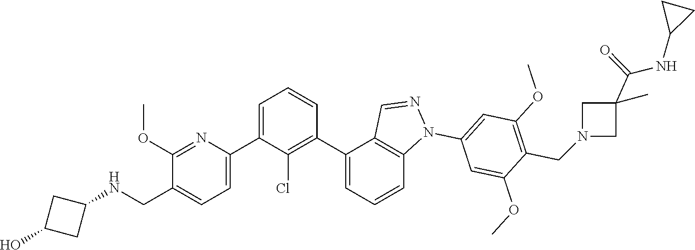 [SMILES: COc1cc(-n2ncc3c(-c4cccc(-c5ccc(CN[C@H]6C[C@@H](O)C6)c(OC)n5)c4Cl)cccc32)cc(OC)c1CN1CC(C)(C(=O)NC2CC2)C1] |
| | 1-(4-(4-(2-chloro-3-(5-(((cis-3-hydroxycyclobutyl)amino)methyl)-6- |
| | methoxypyridin-2-yl)phenyl)-1H-indazol-1-yl)-2,6- |
| | dimethoxybenzyl)-N-cyclopropyl-3-methylazetidine-3-carboxamide |
| 173 | 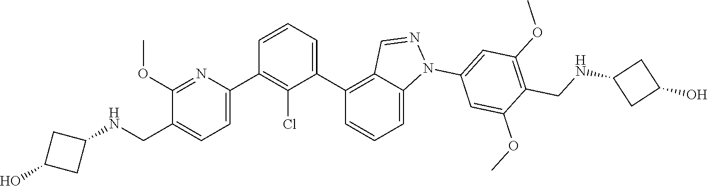 [SMILES: COc1cc(-n2ncc3c(-c4cccc(-c5ccc(CN[C@H]6C[C@@H](O)C6)c(OC)n5)c4Cl)cccc32)cc(OC)c1CN[C@H]1C[C@@H](O)C1] |
| | cis-3-(((6-(2-chloro-3-(1-(4-(((cis-3- |
| | hydroxycyclobutyl)amino)methyl)-3,5-dimethoxyphenyl)-1H-indazol- |
| | 4-yl)phenyl)-2-methoxypyridin-3-yl)methyl)amino)cyclobutanol |
| 174 | 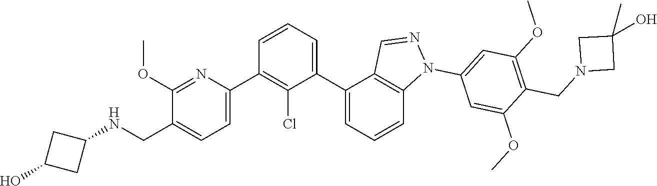 [SMILES: COc1cc(-n2ncc3c(-c4cccc(-c5ccc(CN[C@H]6C[C@@H](O)C6)c(OC)n5)c4Cl)cccc32)cc(OC)c1CN1CC(C)(O)C1] |
| | 1-(4-(4-(2-chloro-3-(5-(((cis-3-hydroxycyclobutyl)amino)methyl)-6- |
| | methoxypyridin-2-yl)phenyl)-1H-indazol-1-yl)-2,6- |
| | dimethoxybenzyl)-3-methylazetidin-3-ol |
| 175 | 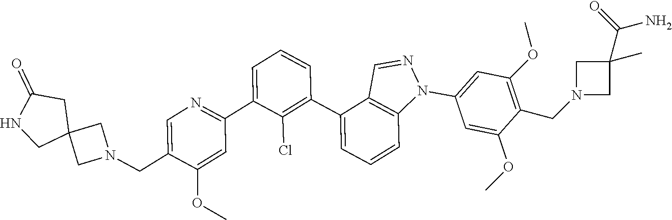 [SMILES: COc1cc(-c2cccc(-c3cccc4c3cnn4-c3cc(OC)c(CN4CC(C)(C(N)=O)C4)c(OC)c3)c2Cl)ncc1CN1CC2(CNC(=O)C2)C1] |
| | 1-(4-(4-(2-chloro-3-(6-methoxy-5-((7-oxo-2,6-diazaspiro[3.4]octan-2- |
| | yl)methyl)pyridin-2-yl)phenyl)-1H-indazol-1-yl)-2,6- |
| | dimethoxybenzyl)-3-methylazetidine-3-carboxamide |
| 176 | 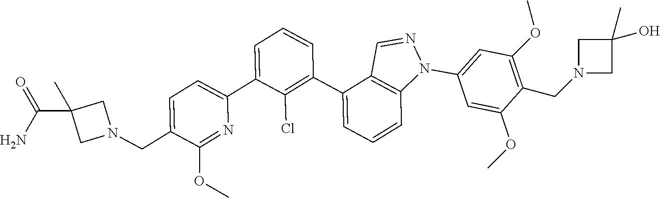 [SMILES: COc1cc(-n2ncc3c(-c4cccc(-c5ccc(CN6CC(C)(C(N)=O)C6)c(OC)n5)c4Cl)cccc32)cc(OC)c1CN1CC(C)(O)C1] |
| | 1-((6-(2-chloro-3-(1-(4-((3-hydroxy-3-methylazetidin-1-yl)methyl)- |
| | 3,5-dimethoxyphenyl)-1H-indazol-4-yl)phenyl)-2-methoxypyridin-3- |
| | yl)methyl)-3-methylazetidine-3-carboxamide |
| 177 | 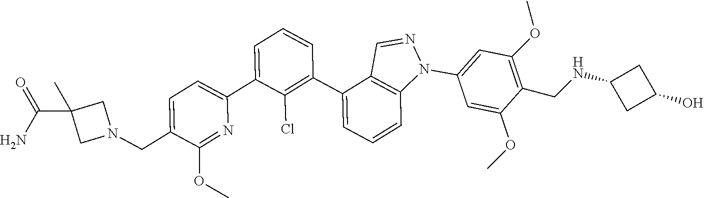 [SMILES: COc1cc(-n2ncc3c(-c4cccc(-c5ccc(CN6CC(C)(C(N)=O)C6)c(OC)n5)c4Cl)cccc32)cc(OC)c1CN[C@H]1C[C@@H](O)C1] |
| | 1-((6-(2-chloro-3-(1-(4-(((cis-3-hydroxycyclobutyl)amino)methyl)- |
| | 3,5-dimethoxyphenyl)-1H-indazol-4-yl)phenyl)-2-methoxypyridin-3- |
| | yl)methyl)-3-methylazetidine-3-carboxamide |
| 178 | 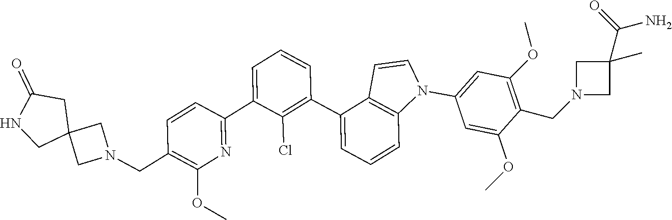 [SMILES: COc1cc(-n2ccc3c(-c4cccc(-c5ccc(CN6CC7(CNC(=O)C7)C6)c(OC)n5)c4Cl)cccc32)cc(OC)c1CN1CC(C)(C(N)=O)C1] |
| | 1-(4-(4-(2-chloro-3-(6-methoxy-5-((7-oxo-2,6-diazaspiro[3.4]octan-2- |
| | yl)methyl)pyridin-2-yl)phenyl)-1H-indol-1-yl)-2,6-dimethoxybenzyl)- |
| | 3-methylazetidine-3-carboxamide |
| 179 | 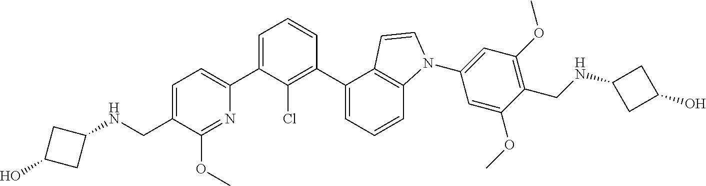 [SMILES: COc1cc(-n2ccc3c(-c4cccc(-c5ccc(CN[C@H]6C[C@@H](O)C6)c(OC)n5)c4Cl)cccc32)cc(OC)c1CN[C@H]1C[C@@H](O)C1] |
| | cis-3-(((6-(2-chloro-3-(1-(4-(((cis-3- |
| | hydroxycyclobutyl)amino)methyl)-3,5-dimethoxyphenyl)-1H-indol-4- |
| | yl)phenyl)-2-methoxypyridin-3-yl)methyl)amino)cyclobutanol |
| 180 | 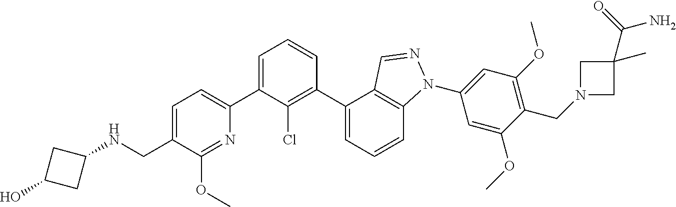 [SMILES: COc1cc(-n2ncc3c(-c4cccc(-c5ccc(CN[C@H]6C[C@@H](O)C6)c(OC)n5)c4Cl)cccc32)cc(OC)c1CN1CC(C)(C(N)=O)C1] |
| | 1-(4-(4-(2-chloro-3-(5-(((cis-3-hydroxycyclobutyl)amino)methyl)-6- |
| | methoxypyridin-2-yl)phenyl)-1H-indazol-1-yl)-2,6- |
| | dimethoxybenzyl)-3-methylazetidine-3-carboxamide |
| 181 | 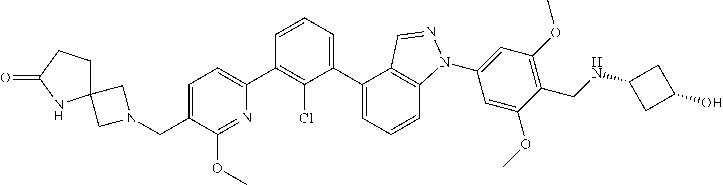 [SMILES: COc1cc(-n2ncc3c(-c4cccc(-c5ccc(CN6CC7(CCC(=O)N7)C6)c(OC)n5)c4Cl)cccc32)cc(OC)c1CN[C@H]1C[C@@H](O)C1] |
| | 2-((6-(2-chloro-3-(1-(4-(((cis-3-hydroxycyclobutyl)amino)methyl)- |
| | 3,5-dimethoxyphenyl)-1H-indazol-4-yl)phenyl)-2-methoxypyridin-3- |
| | yl)methyl)-2,5-diazaspiro[3,4]octan-6-one |
| 182 | 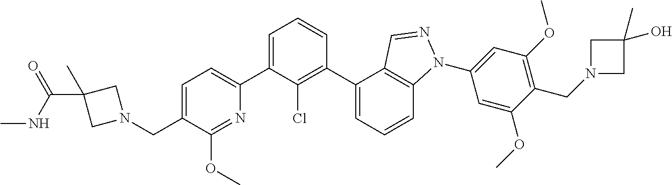 [SMILES: CNC(=O)C1(C)CN(Cc2ccc(-c3cccc(-c4cccc5c4cnn5-c4cc(OC)c(CN5CC(C)(O)C5)c(OC)c4)c3Cl)nc2OC)C1] |
| | 1-((6-(2-chloro-3-(1-(4-((3-hydroxy-3-methylazetidin-1-yl)methyl)- |
| | 3,5-dimethoxyphenyl)-1H-indazol-4-yl)phenyl)-2-methoxypyridin-3- |
| | yl)methyl)-N,3-dimethylazetidine-3-carboxamide |
| 183 | 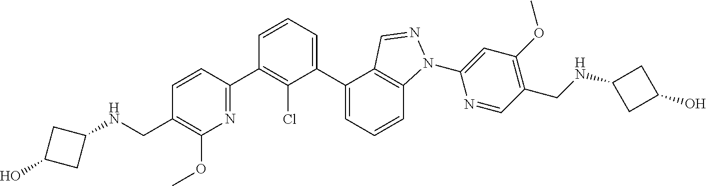 [SMILES: COc1cc(-n2ncc3c(-c4cccc(-c5ccc(CN[C@H]6C[C@@H](O)C6)c(OC)n5)c4Cl)cccc32)ncc1CN[C@H]1C[C@@H](O)C1] |
| | cis-3-(((6-(2-chloro-3-(1-(5-(((cis-3- |
| | hydroxycyclobutyl)amino)methyl)-4-methoxypyridin-2-yl)-1H- |
| | indazol-4-yl)phenyl)-2-methoxypyridin-3- |
| | yl)methyl)amino)cyclobutanol |
| 184 | 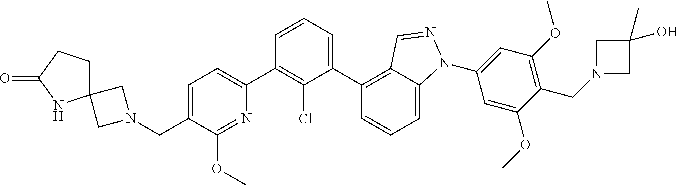 [SMILES: COc1cc(-n2ncc3c(-c4cccc(-c5ccc(CN6CC7(CCC(=O)N7)C6)c(OC)n5)c4Cl)cccc32)cc(OC)c1CN1CC(C)(O)C1] |
| | 2-((6-(2-chloro-3-(1-(4-((3-hydroxy-3-methylazetidin-1-yl)methyl)- |
| | 3,5-dimethoxyphenyl)-1H-indazol-4-yl)phenyl)-2-methoxypyridin-3- |
| | yl)methyl)-2,5-diazaspiro[3.4]octan-6-one |
| 185 | 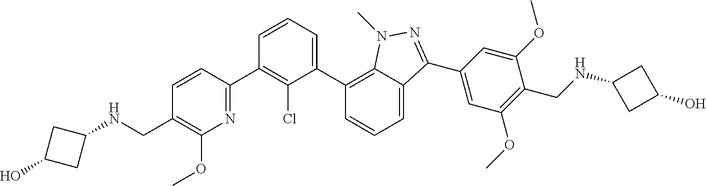 [SMILES: COc1cc(-c2nn(C)c3c(-c4cccc(-c5ccc(CN[C@H]6C[C@@H](O)C6)c(OC)n5)c4Cl)cccc23)cc(OC)c1CN[C@H]1C[C@@H](O)C1] |
| | cis-3-(((6-(2-chloro-3-(3-(4-(((cis-3- |
| | hydroxycyclobutyl)amino)methyl)-3,5-dimethoxyphenyl)-1-methyl- |
| | 1H-indazol-7-yl)phenyl)-2-methoxypyridin-3- |
| | yl)methyl)amino)cyclobutanol |
| 186 | 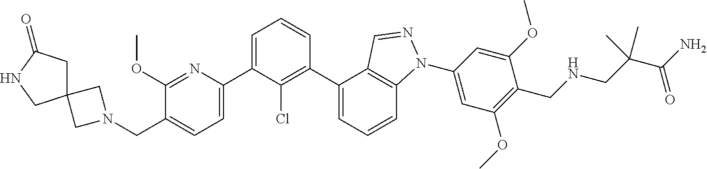 [SMILES: COc1cc(-n2ncc3c(-c4cccc(-c5ccc(CN6CC7(CNC(=O)C7)C6)c(OC)n5)c4Cl)cccc32)cc(OC)c1CNCC(C)(C)C(N)=O] |
| | 3-((4-(4-(2-chloro-3-(6-methoxy-5-((7-oxo-2,6-diazaspiro[3.4]octan- |
| | 2-yl)methyl)pyridin-2-yl)phenyl)-1H-indazol-1-yl)-2,6- |
| | dimethoxybenzyl)amino)-2,2-dimethylpropanamide |
| 187 | 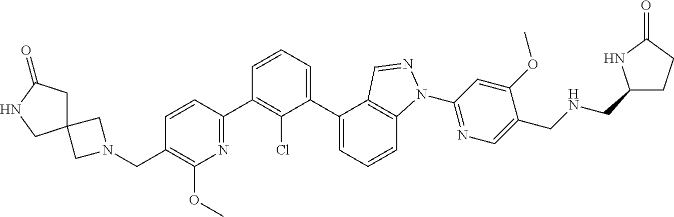 [SMILES: COc1cc(-n2ncc3c(-c4cccc(-c5ccc(CN6CC7(CNC(=O)C7)C6)c(OC)n5)c4Cl)cccc32)ncc1CNC[C@@H]1CCC(=O)N1] |
| | (S)-2-((6-(2-chloro-3-(1-(4-methoxy-5-((((5-oxopyrrolidin-2- |
| | yl)methyl)amino)methyl)pyridin-2-yl)-1H-indazol-4-yl)phenyl)-2- |
| | methoxypyridin-3-yl)methyl)-2,6-diazaspiro[3.4]octan-7-one |
| 188 | 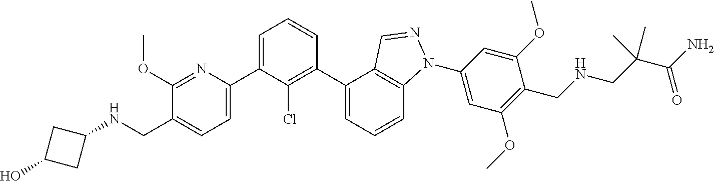 [SMILES: COc1cc(-n2ncc3c(-c4cccc(-c5ccc(CN[C@H]6C[C@@H](O)C6)c(OC)n5)c4Cl)cccc32)cc(OC)c1CNCC(C)(C)C(N)=O] |
| | 3-((4-(4-(2-chloro-3-(5-(((cis-3-hydroxycyclobutyl)amino)methyl)-6- |
| | methoxypyridin-2-yl)phenyl)-1H-indazol-1-yl)-2,6- |
| | dimethoxybenzyl)amino)-2,2-dimethylpropanamide |
| 189 | 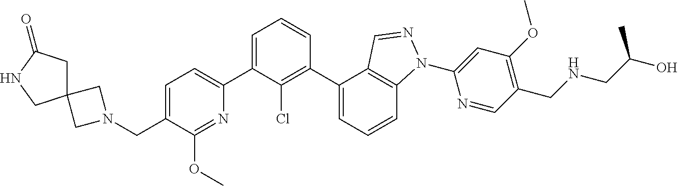 [SMILES: COc1cc(-n2ncc3c(-c4cccc(-c5ccc(CN6CC7(CNC(=O)C7)C6)c(OC)n5)c4Cl)cccc32)ncc1CNC[C@@H](C)O] |
| | (R)-2-((6-(2-chloro-3-(1-(5-(((2-hydroxypropyl)amino)methyl)-4- |
| | methoxypyridin-2-yl)-1H-indazol-4-yl)phenyl)-2-methoxypyridin-3- |
| | yl)methyl)-2,6-diazaspiro[3.4]octan-7-one |
| 190 | 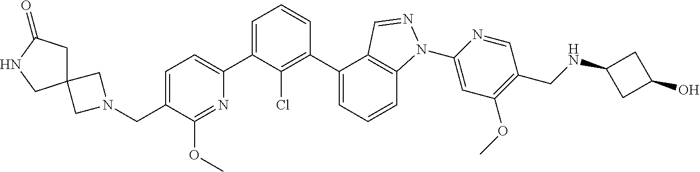 [SMILES: COc1cc(-n2ncc3c(-c4cccc(-c5ccc(CN6CC7(CNC(=O)C7)C6)c(OC)n5)c4Cl)cccc32)ncc1CN[C@H]1C[C@@H](O)C1] |
| | 2-((6-(2-chloro-3-(1-(5-(((cis-3-hydroxycyclobutyl)amino)methyl)-4- |
| | methoxypyridin-2-yl)-1H-indazol-4-yl)phenyl)-2-methoxypyridin-3- |
| | yl)methyl)-2,6-diazaspiro[3.4]octan-7-one |
| 191 | 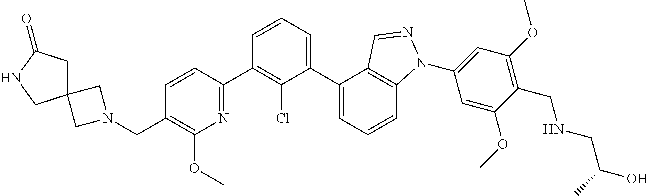 [SMILES: COc1cc(-n2ncc3c(-c4cccc(-c5ccc(CN6CC7(CNC(=O)C7)C6)c(OC)n5)c4Cl)cccc32)cc(OC)c1CNC[C@@H](C)O] |
| | (R)-2-((6-(2-chloro-3-(1-(4-(((2-hydroxypropyl)amino)methyl)-3,5- |
| | dimethoxyphenyl)-1H-indazol-4-yl)phenyl)-2-methoxypyridin-3- |
| | yl)methyl)-2,6-diazaspiro[3.4]octan-7-one |
| 192 | 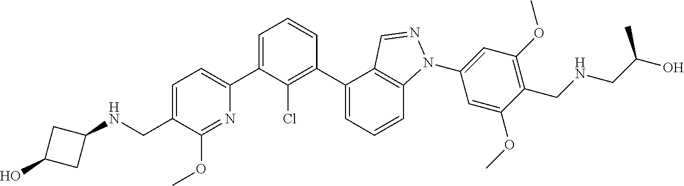 [SMILES: COc1cc(-n2ncc3c(-c4cccc(-c5ccc(CN[C@H]6C[C@@H](O)C6)c(OC)n5)c4Cl)cccc32)cc(OC)c1CNC[C@@H](C)O] |
| | cis-3-(((6-(2-chloro-3-(1-(4-((((R)-2-hydroxypropyl)amino)methyl)- |
| | 3,5-dimethoxyphenyl)-1H-indazol-4-yl)phenyl)-2-methoxypyridin-3- |
| | yl)methyl)amino)cyclobutanol |
| 193 | 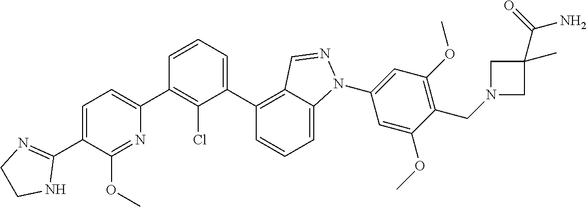 [SMILES: COc1cc(-n2ncc3c(-c4cccc(-c5ccc(C6=NCCN6)c(OC)n5)c4Cl)cccc32)cc(OC)c1CN1CC(C)(C(N)=O)C1] |
| | 1-(4-(4-(2-chloro-3-(5-(4,5-dihydro-1H-imidazol-2-yl)-6- |
| | methoxypyridin-2-yl)phenyl)-1H-indazol-1-yl)-2,6- |
| | dimethoxybenzyl)-3-methylazetidine-3-carboxamide |
| 194 | 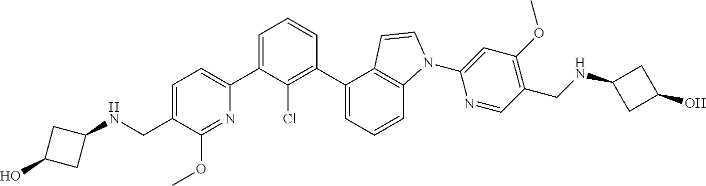 [SMILES: COc1cc(-n2ccc3c(-c4cccc(-c5ccc(CN[C@H]6C[C@@H](O)C6)c(OC)n5)c4Cl)cccc32)ncc1CN[C@H]1C[C@@H](O)C1] |
| | cis-3-(((6-(2-chloro-3-(1-(5-(((cis-3- |
| | hydroxycyclobutyl)amino)methyl)-4-methoxypyridin-2-yl)-1H-indol- |
| | 4-yl)phenyl)-2-methoxypyridin-3-yl)methyl)amino)cyclobutanol |
| 195 | 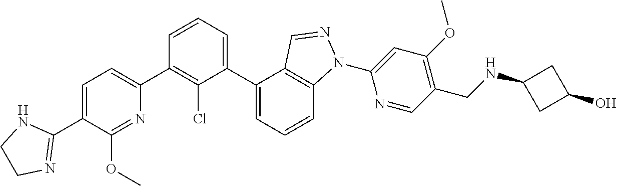 [SMILES: COc1cc(-n2ncc3c(-c4cccc(-c5ccc(C6=NCCN6)c(OC)n5)c4Cl)cccc32)ncc1CN[C@H]1C[C@@H](O)C1] |
| | cis-3-(((6-(4-(2-chloro-3-(5-(4,5-dihydro-1H-imidazol-2-yl)-6- |
| | methoxypyridin-2-yl)phenyl)-1H-indazol-1-yl)-4-methoxypyridin-3- |
| | yl)methyl)amino)cyclobutanol |
| 196 | 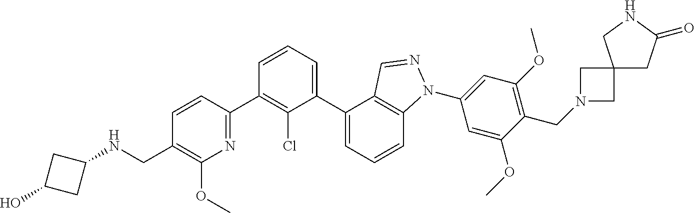 [SMILES: COc1cc(-n2ncc3c(-c4cccc(-c5ccc(CN[C@H]6C[C@@H](O)C6)c(OC)n5)c4Cl)cccc32)cc(OC)c1CN1CC2(CNC(=O)C2)C1] |
| | 2-(4-(4-(2-chloro-3-(5-(((cis-3-hydroxycyclobutyl)amino)methyl)-6- |
| | methoxypyridin-2-yl)phenyl)-1H-indazol-1-yl)-2,6- |
| | dimethoxybenzyl)-2,6-diazaspiro[3.4]octan-7-one |
| 197 | 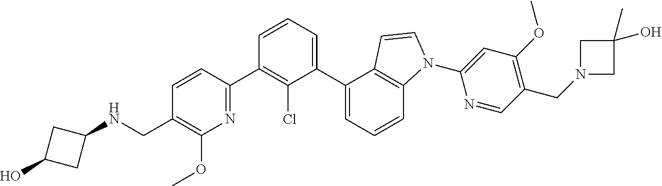 [SMILES: COc1cc(-n2ccc3c(-c4cccc(-c5ccc(CN[C@H]6C[C@@H](O)C6)c(OC)n5)c4Cl)cccc32)ncc1CN1CC(C)(O)C1] |
| | 1-((6-(4-(2-chloro-3-(5-(((cis-3-hydroxycyclobutyl)amino)methyl)-6- |
| | methoxypyridin-2-yl)phenyl)-1H-indol-1-yl)-4-methoxypyridin-3- |
| | yl)methyl)-3-methylazetidin-3-ol |
| 198 | 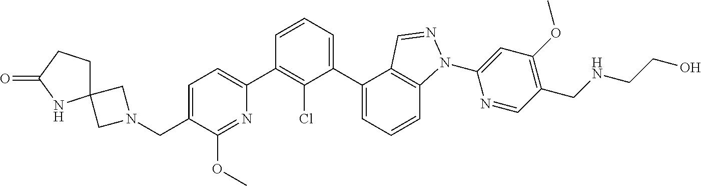 [SMILES: COc1cc(-n2ncc3c(-c4cccc(-c5ccc(CN6CC7(CCC(=O)N7)C6)c(OC)n5)c4Cl)cccc32)ncc1CNCCO] |
| | 2-((6-(2-chloro-3-(1-(5-(((2-hydroxyethyl)amino)methyl)-4- |
| | methoxypyridin-2-yl)-1H-indazol-4-yl)phenyl)-2-methoxypyridin-3- |
| | yl)methyl)-2,5-diazaspiro[3.4]octan-6-one |
| 199 | 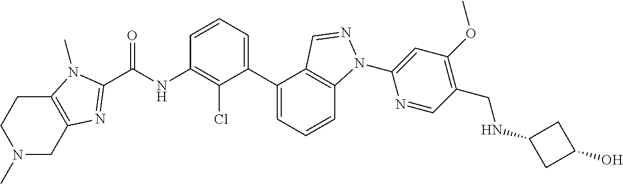 [SMILES: COc1cc(-n2ncc3c(-c4cccc(NC(=O)c5nc6c(n5C)CCN(C)C6)c4Cl)cccc32)ncc1CN[C@H]1C[C@@H](O)C1] |
| | N, N-(2-chloro-3-(1-(5-(((cis-3-hydroxycyclobutyl)amino)methyl)-4- |
| | methoxypyridin-2-yl)-1H-indazol-4-yl)phenyl)-1,5-dimethyl-4,5,6,7- |
| | tetrahydro-1H-imidazo[4,5-c]pyridine-2-carboxamide |
| 200 | 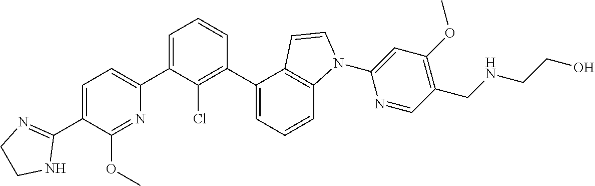 [SMILES: COc1cc(-n2ccc3c(-c4cccc(-c5ccc(C6=NCCN6)c(OC)n5)c4Cl)cccc32)ncc1CNCCO] |
| | 2-(((6-(4-(2-chloro-3-(5-(4,5-dihydro-1H-imidazol-2-yl)-6- |
| | methoxypyridin-2-yl)phenyl)-1H-indol-1-yl)-4-methoxypyridin-3- |
| | yl)methyl)amino)ethanol |
| 201 | 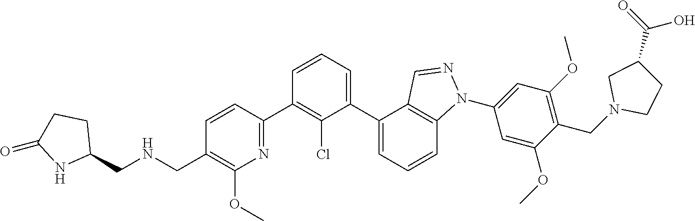 [SMILES: COc1cc(-n2ncc3c(-c4cccc(-c5ccc(CNC[C@@H]6CCC(=O)N6)c(OC)n5)c4Cl)cccc32)cc(OC)c1CN1CC[C@@H](C(=O)O)C1] |
| | (R)-1-(4-(4-(2-chloro-3-(6-methoxy-5-(((((S)-5-oxopyrrolidin-2- |
| | yl)methyl)amino)methyl)pyridin-2-yl)phenyl)-1H-indazol-1-yl)-2,6- |
| | dimethoxybenzyl)pyrrolidine-3-carboxylic acid |
| 202 | 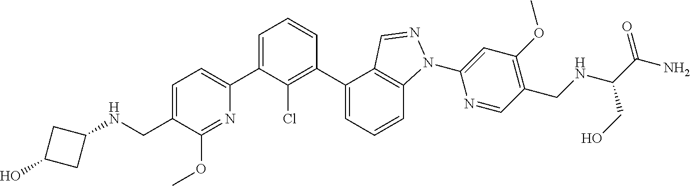 [SMILES: COc1cc(-n2ncc3c(-c4cccc(-c5ccc(CN[C@H]6C[C@@H](O)C6)c(OC)n5)c4Cl)cccc32)ncc1CN[C@@H](CO)C(N)=O] |
| | (S)-2-(((6-(4-(2-chloro-3-(5-(((cis-3- |
| | hydroxycyclobutyl)amino)methyl)-6-methoxypyridin-2-yl)phenyl)- |
| | 1H-indazol-1-yl)-4-methoxypyridin-3-yl)methyl)amino)-3- |
| | hydroxypropanamide |
| 203 | 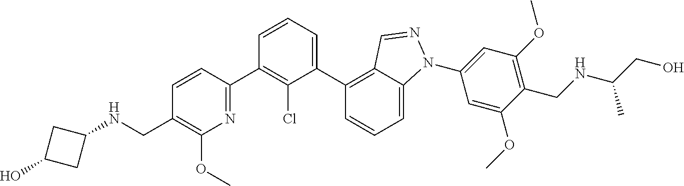 [SMILES: COc1cc(-n2ncc3c(-c4cccc(-c5ccc(CN[C@H]6C[C@@H](O)C6)c(OC)n5)c4Cl)cccc32)cc(OC)c1CN[C@@H](C)CO] |
| | cis-3-(((6-(2-chloro-3-(1-(4-((((S)-1-hydroxypropan-2- |
| | yl)amino)methyl)-3,5-dimethoxyphenyl)-1H-indazol-4-yl)phenyl)-2- |
| | methoxypyridin-3-yl)methyl)amino)cyclobutanol |
| 204 | 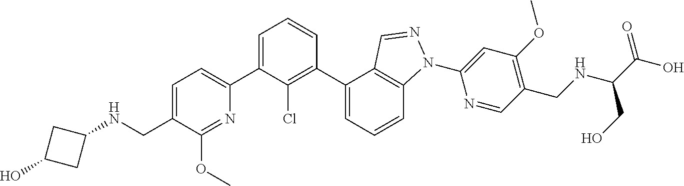 [SMILES: COc1cc(-n2ncc3c(-c4cccc(-c5ccc(CN[C@H]6C[C@@H](O)C6)c(OC)n5)c4Cl)cccc32)ncc1CN[C@H](CO)C(=O)O] |
| | ((6-(4-(2-chloro-3-(5-(((cis-3-hydroxycyclobutyl)amino)methyl)-6- |
| | methoxypyridin-2-yl)phenyl)-1H-indazol-1-yl)-4-methoxypyridin-3- |
| | yl)methyl)-D-serine |
| 205 | 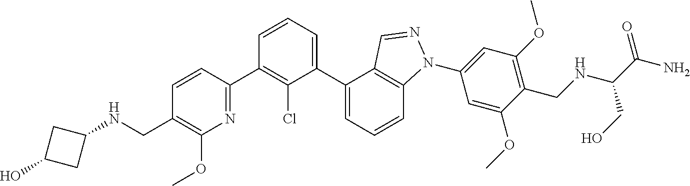 [SMILES: COc1cc(-n2ncc3c(-c4cccc(-c5ccc(CN[C@H]6C[C@@H](O)C6)c(OC)n5)c4Cl)cccc32)cc(OC)c1CN[C@@H](CO)C(N)=O] |
| | (S)-2-((4-(4-(2-chloro-3-(5-(((cis-3- |
| | hydroxycyclobutyl)amino)methyl)-6-methoxypyridin-2-yl)phenyl)- |
| | 1H-indazol-1-yl)-2,6-dimethoxybenzyl)amino)-3- |
| | hydroxypropanamide |
| 206 | 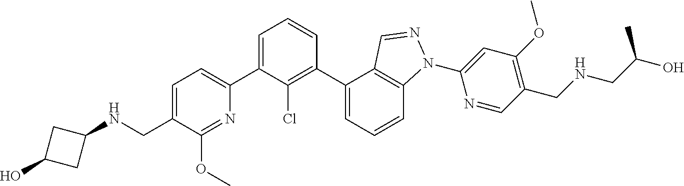 [SMILES: COc1cc(-n2ncc3c(-c4cccc(-c5ccc(CN[C@H]6C[C@@H](O)C6)c(OC)n5)c4Cl)cccc32)ncc1CNC[C@@H](C)O] |
| | cis-3-(((6-(2-chloro-3-(1-(5-((((R)-2-hydroxypropyl)amino)methyl)-4- |
| | methoxypyridin-2-yl)-1H-indazol-4-yl)phenyl)-2-methoxypyridin-3- |
| | yl)methyl)amino)cyclobutanol |
| 207 | 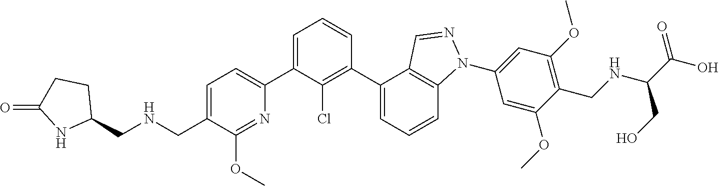 [SMILES: COc1cc(-n2ncc3c(-c4cccc(-c5ccc(CNC[C@@H]6CCC(=O)N6)c(OC)n5)c4Cl)cccc32)cc(OC)c1CN[C@H](CO)C(=O)O] |
| | (4-(4-(2-chloro-3-(6-methoxy-5-(((((S)-5-oxopyrrolidin-2- |
| | yl)methyl)amino)methyl)pyridin-2-yl)phenyl)-1H-indazol-1-yl)-2,6- |
| | dimethoxybenzyl)-D-serine |
| 208 | 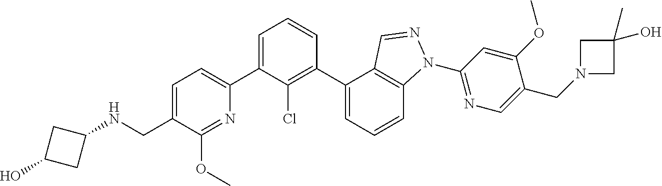 [SMILES: COc1cc(-n2ncc3c(-c4cccc(-c5ccc(CN[C@H]6C[C@@H](O)C6)c(OC)n5)c4Cl)cccc32)ncc1CN1CC(C)(O)C1] |
| | 1-((6-(4-(2-chloro-3-(5-(((cis-3-hydroxycyclobutyl)amino)methyl)-6- |
| | methoxypyridin-2-yl)phenyl)-1H-indazol-1-yl)-4-methoxypyridin-3- |
| | yl)methyl)-3-methylazetidin-3-ol |
| 209 | 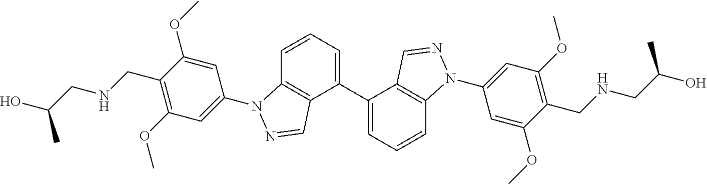 [SMILES: COc1cc(-n2ncc3c(-c4cccc5c4cnn5-c4cc(OC)c(CNC[C@@H](C)O)c(OC)c4)cccc32)cc(OC)c1CNC[C@@H](C)O] |
| | (2R,2′R)-1,l′-(((1H, l′H-[4,4′-biindazole]-1,1′-diylbis(2,6-dimethoxy- |
| | 4,1-phenylene))bis(methylene))bis(azanediyl))bis(propan-2-ol) |
| 210 | 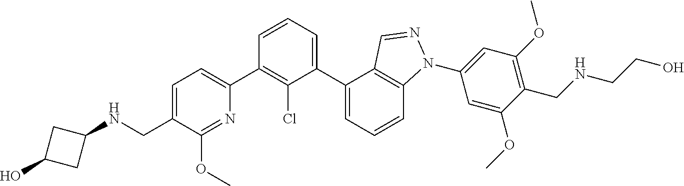 [SMILES: COc1cc(-n2ncc3c(-c4cccc(-c5ccc(CN[C@H]6C[C@@H](O)C6)c(OC)n5)c4Cl)cccc32)cc(OC)c1CNCCO] |
| | cis-3-(((6-(2-chloro-3-(1-(4-(((2-hydroxyethyl)amino)methyl)-3,5- |
| | dimethoxyphenyl)-1H-indazol-4-yl)phenyl)-2-methoxypyridin-3- |
| | yl)methyl)amino)cyclobutanol |
| 211 | 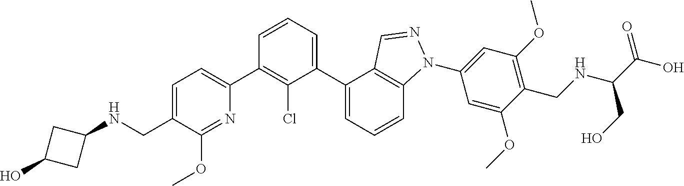 [SMILES: COc1cc(-n2ncc3c(-c4cccc(-c5ccc(CN[C@H]6C[C@@H](O)C6)c(OC)n5)c4Cl)cccc32)cc(OC)c1CN[C@H](CO)C(=O)O] |
| | (4-(4-(2-chloro-3-(5-(((cis-3-hydroxycyclobutyl)amino)methyl)-6- |
| | methoxypyridin-2-yl)phenyl)-1H-indazol-1-yl)-2,6- |
| | dimethoxybenzyl)-D-serine |
| 212 | 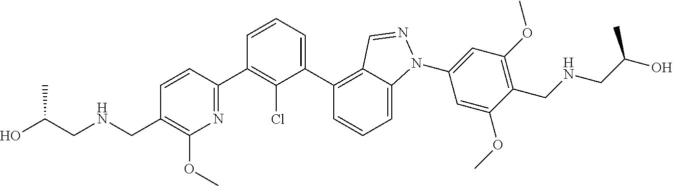 [SMILES: COc1cc(-n2ncc3c(-c4cccc(-c5ccc(CNC[C@@H](C)O)c(OC)n5)c4Cl)cccc32)cc(OC)c1CNC[C@@H](C)O] |
| | (R)-1-(((6-(2-chloro-3-(1-(4-((((R)-2-hydroxypropyl)amino)methyl)- |
| | 3,5-dimethoxyphenyl)-1H-indazol-4-yl)phenyl)-2-methoxypyridin-3- |
| | yl)methyl)amino)propan-2-ol |
| 213 | 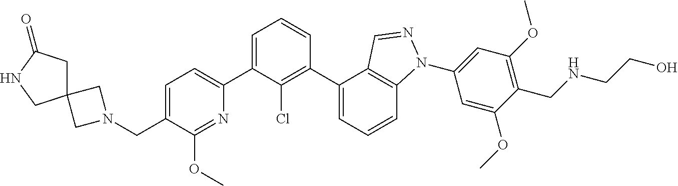 [SMILES: COc1cc(-n2ncc3c(-c4cccc(-c5ccc(CN6CC7(CNC(=O)C7)C6)c(OC)n5)c4Cl)cccc32)cc(OC)c1CNCCO] |
| | 2-((6-(2-chloro-3-(1-(4-(((2-hydroxyethyl)amino)methyl)-3,5- |
| | dimethoxyphenyl)-1H-indazol-4-yl)phenyl)-2-methoxypyridin~3~ |
| | yl)methyl)-2,6-diazaspiro[3.4]octan-7-one |
| 214 | 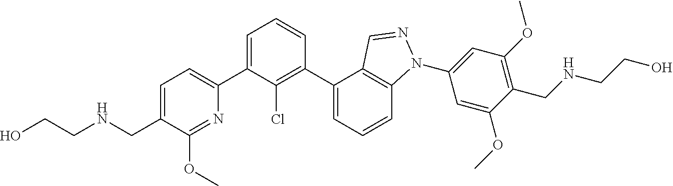 [SMILES: COc1cc(-n2ncc3c(-c4cccc(-c5ccc(CNCCO)c(OC)n5)c4Cl)cccc32)cc(OC)c1CNCCO] |
| | 2-(((6-(2-chloro-3-(1-(4-(((2-hydroxyethyl)amino)methyl)-3,5- |
| | dimethoxyphenyl)-1H-indazol-4-yl)phenyl)-2-methoxypyridin-3- |
| | yl)methyl)amino)ethanol |
| 215 | 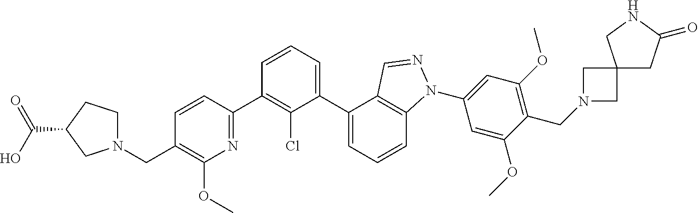 [SMILES: COc1cc(-n2ncc3c(-c4cccc(-c5ccc(CN6CC[C@@H](C(=O)O)C6)c(OC)n5)c4Cl)cccc32)cc(OC)c1CN1CC2(CNC(=O)C2)C1] |
| | (R)-1-((6-(2-chloro-3-(1-(3,5-dimethoxy-4-((7-oxo-2,6- |
| | diazaspiro[3.4]octan-2-yl)methyl)phenyl)-1H-indazol-4-yl)phenyl)-2- |
| | methoxypyridin-3-yl)methyl)pyrrolidine-3-carboxylic acid |
| 216 | 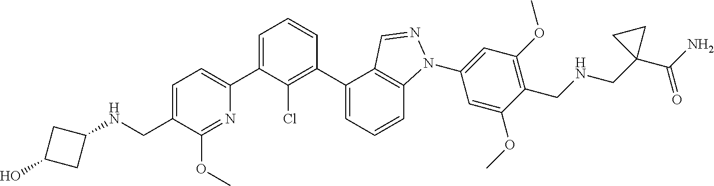 [SMILES: COc1cc(-n2ncc3c(-c4cccc(-c5ccc(CN[C@H]6C[C@@H](O)C6)c(OC)n5)c4Cl)cccc32)cc(OC)c1CNCC1(C(N)=O)CC1] |
| | 1-(((4-(4-(2-chloro-3-(5-(((cis-3-hydroxycyclobutyl)amino)methyl)-6- |
| | methoxypyridin-2-yl)phenyl)-1H-indazol-1-yl)-2,6- |
| | dimethoxybenzyl)amino)methyl)cyclopropane-1-carboxamide |
| 217 | 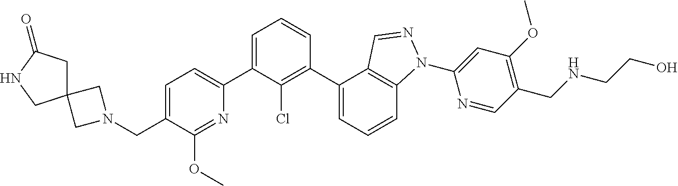 [SMILES: COc1cc(-n2ncc3c(-c4cccc(-c5ccc(CN6CC7(CNC(=O)C7)C6)c(OC)n5)c4Cl)cccc32)ncc1CNCCO] |
| | 2-((6-(2-chloro-3-(1-(5-(((2-hydroxyethyl)amino)methyl)-4- |
| | methoxypyridin-2-yl)-1H-indazol-4-yl)phenyl)-2-methoxypyridin-3- |
| | yl)methyl)-2,6-diazaspiro[3.4]octan-7-one |
| 218 | 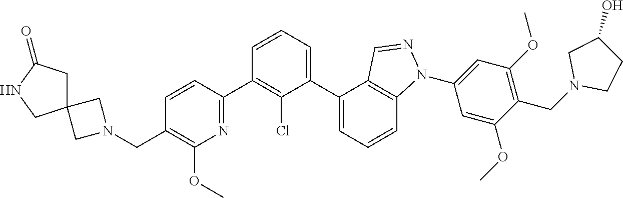 [SMILES: COc1cc(-n2ncc3c(-c4cccc(-c5ccc(CN6CC7(CNC(=O)C7)C6)c(OC)n5)c4Cl)cccc32)cc(OC)c1CN1CC[C@@H](O)C1] |
| | (R)-2-((6-(2-chloro-3-(1-(4-((3-hydroxypyrrolidin-1-yl)methyl)-3,5- |
| | dimethoxyphenyl)-1H-indazol-4-yl)phenyl)-2-methoxypyridin-3- |
| | yl)methyl)-2,6-diazaspiro[3.4]octan-7-one |
| 219 | 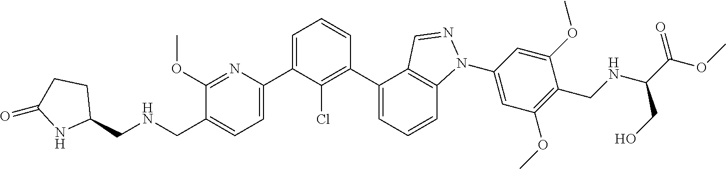 [SMILES: COC(=O)[C@@H](CO)NCc1c(OC)cc(-n2ncc3c(-c4cccc(-c5ccc(CNC[C@@H]6CCC(=O)N6)c(OC)n5)c4Cl)cccc32)cc1OC] |
| | methyl (4-(4-(2-chloro-3-(6-methoxy-5-(((((S)-5-oxopyrrolidin-2- |
| | yl)methyl)amino)methyl)pyridin-2-yl)phenyl)-1H-indazol-1-yl)-2,6- |
| | dimethoxybenzyl)-D-serinate |
| 220 | 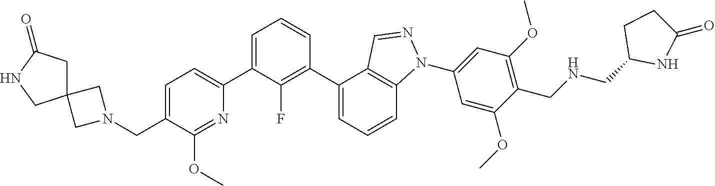 [SMILES: COc1cc(-n2ncc3c(-c4cccc(-c5ccc(CN6CC7(CNC(=O)C7)C6)c(OC)n5)c4F)cccc32)cc(OC)c1CNC[C@@H]1CCC(=O)N1] |
| | (S)-2-((6-(3-(1-(3,5-dimethoxy-4-((((5-oxopyrrolidin-2- |
| | yl)methyl)amino)methyl)phenyl)-1H-indazol-4-yl)-2-fluorophenyl)-2- |
| | methoxypyridin-3-yl)methyl)-2,6-diazaspiro[3.4]octan-7-one |
| 221 | 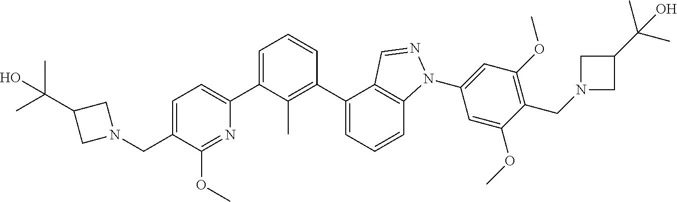 [SMILES: COc1cc(-n2ncc3c(-c4cccc(-c5ccc(CN6CC(C(C)(C)O)C6)c(OC)n5)c4C)cccc32)cc(OC)c1CN1CC(C(C)(C)O)C1] |
| | 2-(1-((6-(3-(1-(4-((3-(2-hydroxypropan-2-yl)azetidin-1-yl)methyl)- |
| | 3,5-dimethoxyphenyl)-1H-indazol-4-yl)-2-methylphenyl)-2- |
| | methoxypyridin-3-yl)methyl)azetidin-3-yl)propan-2-ol |
| 222 | 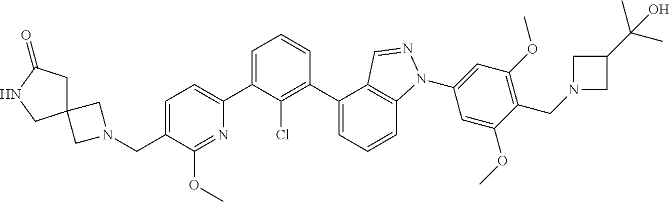 [SMILES: COc1cc(-n2ncc3c(-c4cccc(-c5ccc(CN6CC7(CNC(=O)C7)C6)c(OC)n5)c4Cl)cccc32)cc(OC)c1CN1CC(C(C)(C)O)C1] |
| | 2-((6-(2-chloro-3-(1-(4-((3-(2-hydroxypropan-2-yl)azetidin-1- |
| | yl)methyl)-3,5-dimethoxyphenyl)-1H-indazol-4-yl)phenyl)-2- |
| | methoxypyridin-3-yl)methyl)-2,6-diazaspiro[3.4]octan-7-one |
| 223 | 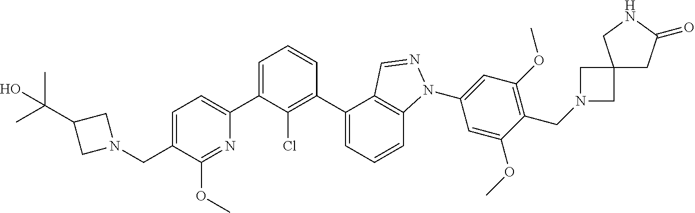 [SMILES: COc1cc(-n2ncc3c(-c4cccc(-c5ccc(CN6CC(C(C)(C)O)C6)c(OC)n5)c4Cl)cccc32)cc(OC)c1CN1CC2(CNC(=O)C2)C1] |
| | 2-(4-(4-(2-chloro-3-(5-((3-(2-hydroxypropan-2-yl)azetidin-1- |
| | yl)methyl)-6-methoxypyridin-2-yl)phenyl)-1H-indazol-1-yl)-2,6- |
| | dimethoxybenzyl)-2,6-diazaspiro[3.4]octan-7-one |
| 224 | 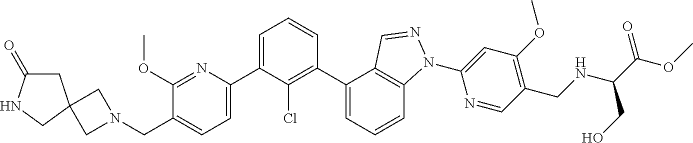 [SMILES: COC(=O)[C@@H](CO)NCc1cnc(-n2ncc3c(-c4cccc(-c5ccc(CN6CC7(CNC(=O)C7)C6)c(OC)n5)c4Cl)cccc32)cc1OC] |
| | (R)-methyl 2-(((6-(4-(2-chloro-3-(6-methoxy-5-((7-oxo-2,6- |
| | diazaspiro[3.4]octan-2-yl)methyl)pyridin-2-yl)phenyl)-1H-indazol-1- |
| | yl)-4-methoxypyridin~3-yl)methyl)amino)-3-hydroxypropanoate |
| 225 | 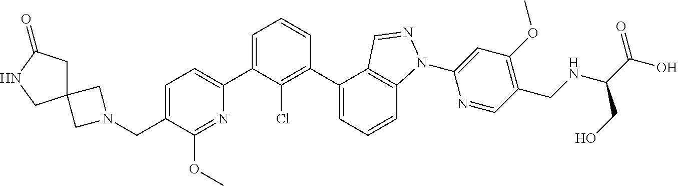 [SMILES: COc1cc(-n2ncc3c(-c4cccc(-c5ccc(CN6CC7(CNC(=O)C7)C6)c(OC)n5)c4Cl)cccc32)ncc1CN[C@H](CO)C(=O)O] |
| | (R)-2-(((6-(4-(2-chloro-3-(6-methoxy-5-((7-oxo-2,6- |
| | diazaspiro[3.4]octan-2-yl)methyl)pyridin-2-yl)phenyl)-1H-indazol-1- |
| | yl)-4-methoxypyridin-3-yl)methyl)amino)-3-hydroxypropanoic acid |
| 226 | 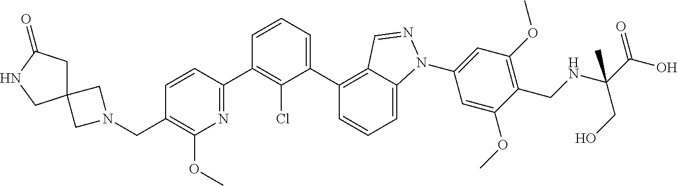 [SMILES: COc1cc(-n2ncc3c(-c4cccc(-c5ccc(CN6CC7(CNC(=O)C7)C6)c(OC)n5)c4Cl)cccc32)cc(OC)c1CN[C@@](C)(CO)C(=O)O] |
| | (S)-2-((4-(4-(2-chloro-3-(6-methoxy-5-((7-oxo-2,6- |
| | diazaspiro[3.4]octan-2-yl)methyl)pyridin-2-yl)phenyl)-1H-indazol-1- |
| | yl)-2,6-dimethoxybenzyl)amino)-3-hydroxy-2-methylpropanoic acid |
| 227 | 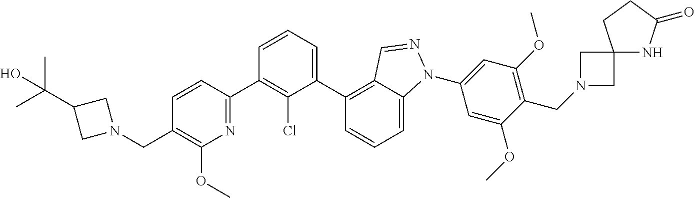 [SMILES: COc1cc(-n2ncc3c(-c4cccc(-c5ccc(CN6CC(C(C)(C)O)C6)c(OC)n5)c4Cl)cccc32)cc(OC)c1CN1CC2(CCC(=O)N2)C1] |
| | 2-(4-(4-(2-chloro-3-(5-((3-(2-hydroxypropan-2-yl)azetidin-1- |
| | yl)methyl)-6-methoxypyridin-2-yl)phenyl)-1H-indazol-1-yl)-2,6- |
| | dimethoxybenzyl)-2,5-diazaspiro[3.4]octan-6-one |
| 228 | 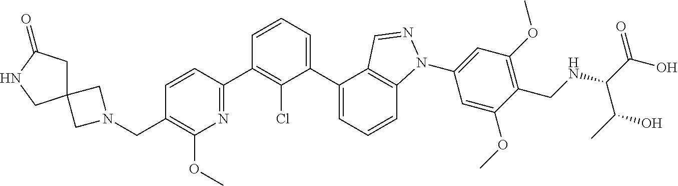 [SMILES: COc1cc(-n2ncc3c(-c4cccc(-c5ccc(CN6CC7(CNC(=O)C7)C6)c(OC)n5)c4Cl)cccc32)cc(OC)c1CN[C@H](C(=O)O)[C@@H](C)O] |
| | (4-(4-(2-chloro-3-(6-methoxy-5-((7-oxo-2,6-diazaspiro[3.4]octan-2- |
| | yl)methyl)pyridin-2-yl)phenyl)-1H-indazol-1-yl)-2,6- |
| | dimethoxybenzyl)-L-threonine |
| 229 | 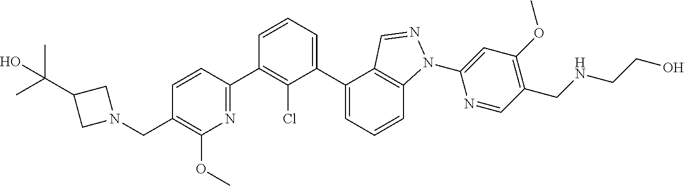 [SMILES: COc1cc(-n2ncc3c(-c4cccc(-c5ccc(CN6CC(C(C)(C)O)C6)c(OC)n5)c4Cl)cccc32)ncc1CNCCO] |
| | 2-(1-((6-(2-chloro-3-(1-(5-(((2-hydroxyethyl)amino)methyl)-4- |
| | methoxypyridin-2-yl)-1H-indazol-4-yl)phenyl)-2-methoxypyridin-3- |
| | yl)methyl)azetidin-3-yl)propan-2-ol |
| 230 | 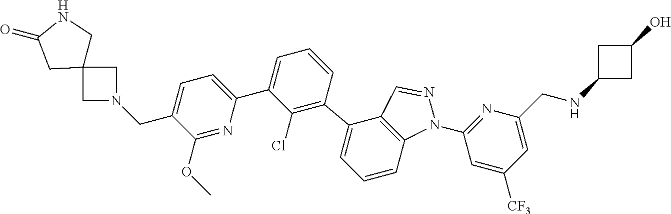 [SMILES: COc1nc(-c2cccc(-c3cccc4c3cnn4-c3cc(C(F)(F)F)cc(CN[C@H]4C[C@@H](O)C4)n3)c2Cl)ccc1CN1CC2(CNC(=O)C2)C1] |
| | 2-((6-(2-chloro-3-(1-(6-((((cis)-3-hydroxycyclobutyl)amino)methyl)- |
| | 4-(trifluoromethyl)pyridin-2-yl)-1H-indazol-4-yl)phenyl)-2- |
| | methoxypyridin-3-yl)methyl)-2,6-diazaspiro[3.4]octan-7-one |
| 231 | 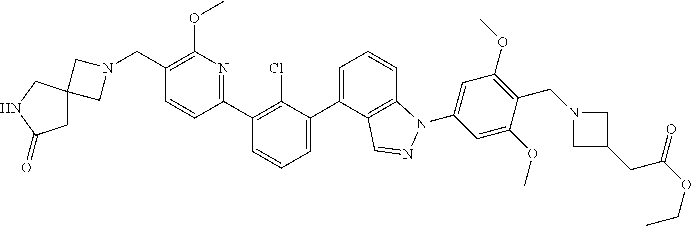 [SMILES: CCOC(=O)CC1CN(Cc2c(OC)cc(-n3ncc4c(-c5cccc(-c6ccc(CN7CC8(CNC(=O)C8)C7)c(OC)n6)c5Cl)cccc43)cc2OC)C1] |
| | ethyl 2-(1-(4-(4-(2-chloro-3-(6-methoxy-5-((7-oxo-2,6- |
| | diazaspiro[3.4]octan-2-yl)methyl)pyridin-2-yl)phenyl)-1H-indazol-1- |
| | yl)-2,6-dimethoxybenzyl)azetidin-3-yl)acetate |
| 232 | 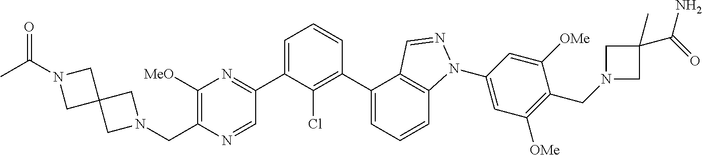 [SMILES: COc1cc(-n2ncc3c(-c4cccc(-c5cnc(CN6CC7(C6)CN(C(C)=O)C7)c(OC)n5)c4Cl)cccc32)cc(OC)c1CN1CC(C)(C(N)=O)C1] |
| | 1-(4-(4-(3-(5-((6-acetyl-2,6-diazaspiro[3.3]heptan-2-y])methyl)-6- |
| | methoxypyrazin-2-yl)-2-chlorophenyl)-1H-indazol-1-yl)-2,6- |
| | dimethoxybenzyl)-3-methylazetidine-3-carboxamide |
| 233 | 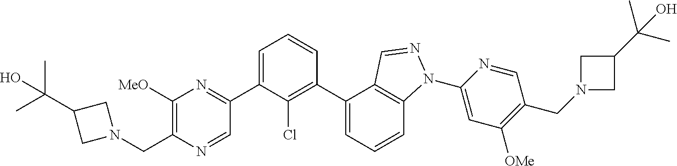 [SMILES: COc1cc(-n2ncc3c(-c4cccc(-c5cnc(CN6CC(C(C)(C)O)C6)c(OC)n5)c4Cl)cccc32)ncc1CN1CC(C(C)(C)O)C1] |
| | 2-(1-((5-(2-chloro-3-(1-(5-((3-(2-hydroxypropan-2-yl)azetidin-1- |
| | yl)methyl)-4-methoxypyridin-2-yl)-1H-indazol-4-yl)phenyl)-3- |
| | methoxypyrazin-2-yl)methyl)azetidin~3-yl)propan-2-ol |
| 234 | 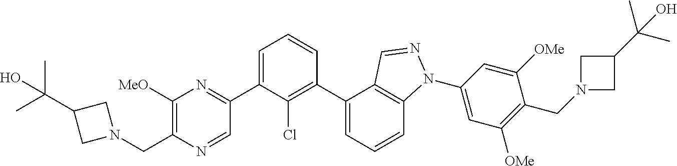 [SMILES: COc1cc(-n2ncc3c(-c4cccc(-c5cnc(CN6CC(C(C)(C)O)C6)c(OC)n5)c4Cl)cccc32)cc(OC)c1CN1CC(C(C)(C)O)C1] |
| | 2-(1-((5-(2-chloro-3-(1-(4-((3-(2-hydroxypropan-2-yl)azetidin-1- |
| | yl)methyl)-3,5-dimethoxyphenyl)-1H-indazol-4-yl)phenyl)-3- |
| | methoxypyrazin-2-yl)methyl)azetidin-3-yl)propan-2-ol |
| 235 | 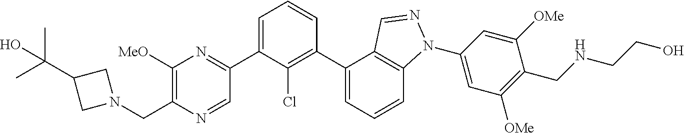 [SMILES: COc1cc(-n2ncc3c(-c4cccc(-c5cnc(CN6CC(C(C)(C)O)C6)c(OC)n5)c4Cl)cccc32)cc(OC)c1CNCCO] |
| | 2-(1-((5-(2-chloro-3-(1-(4-(((2-hydroxyethyl)amino)methyl)-3,5- |
| | dimethoxyphenyl)-1H-indazol-4-yl)phenyl)-3-methoxypyrazin-2- |
| | yl)methyl)azetidin-3-yl)propan-2-ol |
| 236 | 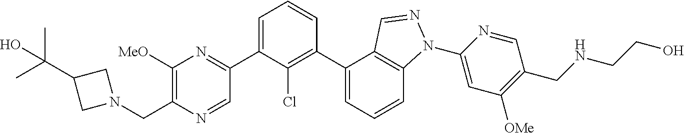 [SMILES: COc1cc(-n2ncc3c(-c4cccc(-c5cnc(CN6CC(C(C)(C)O)C6)c(OC)n5)c4Cl)cccc32)ncc1CNCCO] |
| | 2-(1-((5-(2-chloro-3-(1-(5-(((2-hydroxyethyl)amino)methyl)-4- |
| | methoxypyridin-2-yl)-1H-indazol-4-yl)phenyl)-3-methoxypyrazin-2- |
| | yl)methyl)azetidin-3-yl)propan-2-ol |
| 237 | 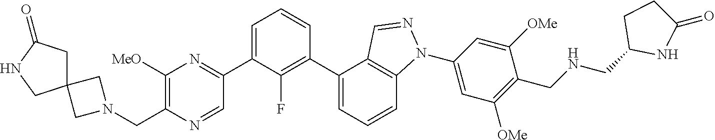 [SMILES: COc1cc(-n2ncc3c(-c4cccc(-c5cnc(CN6CC7(CNC(=O)C7)C6)c(OC)n5)c4F)cccc32)cc(OC)c1CNC[C@@H]1CCC(=O)N1] |
| | (S)-2-((5-(3-(1-(3,5-dimethoxy-4-((((5-oxopyrrolidin-2- |
| | yl)methyl)amino)methyl)phenyl)-1H-indazol-4-yl)-2-fluorophenyl)-3- |
| | methoxypyrazin-2-yl)methyl)-2,6-diazaspiro[3.4]octan-7-one |
| 238 | 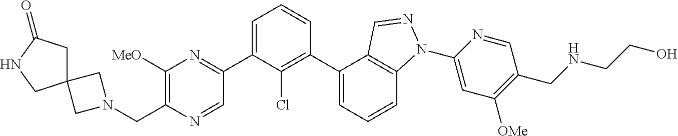 [SMILES: COc1cc(-n2ncc3c(-c4cccc(-c5cnc(CN6CC7(CNC(=O)C7)C6)c(OC)n5)c4Cl)cccc32)ncc1CNCCO] |
| | 2-((5-(2-chloro-3-(1-(5-(((2-hydroxyethyl)amino)methyl)-4- |
| | methoxypyridin-2-yl)-1H-indazol-4-yl)phenyl)-3-methoxypyrazin-2- |
| | yl)methyl)-2,6-diazaspiro[3.4]octan-7-one |
| 239 | 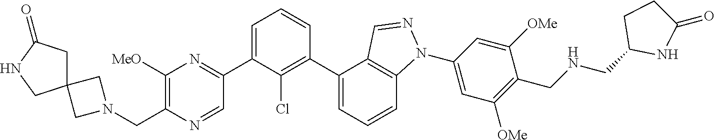 [SMILES: COc1cc(-n2ncc3c(-c4cccc(-c5cnc(CN6CC7(CNC(=O)C7)C6)c(OC)n5)c4Cl)cccc32)cc(OC)c1CNC[C@@H]1CCC(=O)N1] |
| | (S)-2-((5-(2-chloro-3-(1-(3,5-dimethoxy-4-((((5-oxopyrrolidin-2- |
| | yl)methyl)amino)methyl)phenyl)-1H-indazol-4-yl)phenyl)-3- |
| | methoxypyrazin-2-yl)methyl)-2,6-diazaspiro[3.4]octan-7-one |
| 240 | 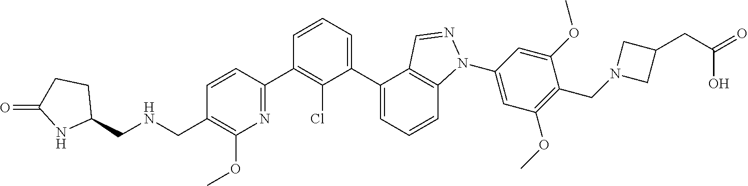 [SMILES: COc1cc(-n2ncc3c(-c4cccc(-c5ccc(CNC[C@@H]6CCC(=O)N6)c(OC)n5)c4Cl)cccc32)cc(OC)c1CN1CC(CC(=O)O)C1] |
| | (S)-2-(1-(4-(4-(2-chloro-3-(6-methoxy-5-((((5-oxopyrrolidin-2- |
| | yl)methyl)amino)methyl)pyridin-2-yl)phenyl)-1H-indazol-1-yl)-2,6- |
| | dimethoxybenzyl)azetidin-3-yl)acetic acid |
| 241 | 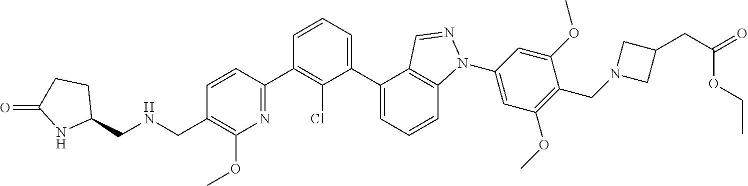 [SMILES: CCOC(=O)CC1CN(Cc2c(OC)cc(-n3ncc4c(-c5cccc(-c6ccc(CNC[C@@H]7CCC(=O)N7)c(OC)n6)c5Cl)cccc43)cc2OC)C1] |
| | ethyl (S)-2-(1-(4-(4-(2-chloro-3-(6-methoxy-5-((((5-oxopyrrolidin-2- |
| | yl)methyl)amino)methyl)pyridin-2-yl)phenyl)-1H-indazol-1-yl)-2,6- |
| | dimethoxybenzyl)azetidin-3-yl)acetate |
| 242 | 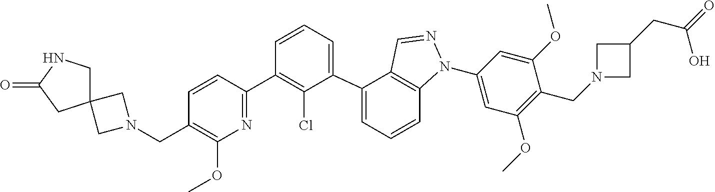 [SMILES: COc1cc(-n2ncc3c(-c4cccc(-c5ccc(CN6CC7(CNC(=O)C7)C6)c(OC)n5)c4Cl)cccc32)cc(OC)c1CN1CC(CC(=O)O)C1] |
| | 2-(1-(4-(4-(2-chloro-3-(6-methoxy-5-((7-oxo-2,6- |
| | diazaspiro[3.4]octan-2-yl)methyl)pyridin-2-yl)phenyl)-1H-indazol-1- |
| | yl)-2,6-dimethoxybenzyl)azetidin-3-yl)acetic acid |
| 243 | 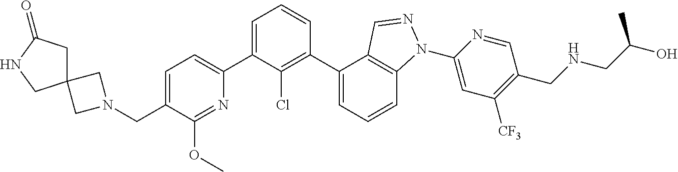 [SMILES: COc1nc(-c2cccc(-c3cccc4c3cnn4-c3cc(C(F)(F)F)c(CNC[C@@H](C)O)cn3)c2Cl)ccc1CN1CC2(CNC(=O)C2)C1] |
| | (R)-2-((6-(2-chloro-3-(1-(5-(((2-hydroxypropyl)amino)methyl)-4- |
| | (trifluoromethyl)pyridin-2-yl)-1H-indazol-4-yl)phenyl)-2- |
| | methoxypyridin-3-yl)methyl)-2,6-diazaspiro[3.4]octan-7-one |
| 244 | 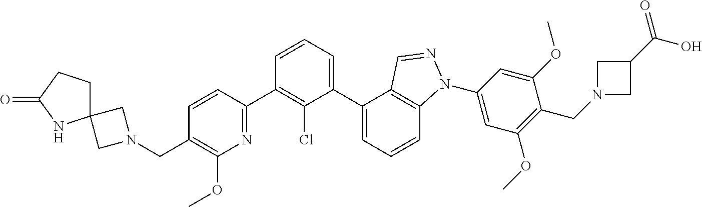 [SMILES: COc1cc(-n2ncc3c(-c4cccc(-c5ccc(CN6CC7(CCC(=O)N7)C6)c(OC)n5)c4Cl)cccc32)cc(OC)c1CN1CC(C(=O)O)C1] |
| | 1-(4-(4-(2-chloro-3-(6-methoxy-5-((6-oxo-2,5-diazaspiro[3.4]octan-2- |
| | yl)methyl)pyridin-2-yl)phenyl)-1H-indazol-1-yl)-2,6- |
| | dimethoxybenzyl)azetidine-3-carboxylic acid |
| 245 | 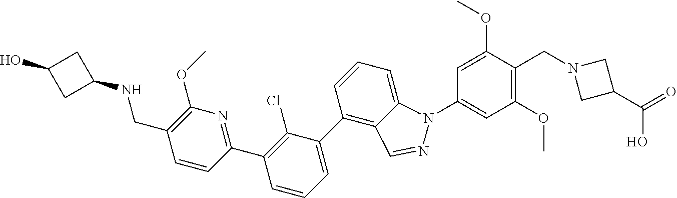 [SMILES: COc1cc(-n2ncc3c(-c4cccc(-c5ccc(CN[C@H]6C[C@@H](O)C6)c(OC)n5)c4Cl)cccc32)cc(OC)c1CN1CC(C(=O)O)C1] |
| | 1-(4-(4-(2-chloro-3-(5-((((cis)-3-hydroxycyclobutyl)amino)methyl)-6- |
| | methoxypyridin-2-yl)phenyl)-1H-indazol-1-yl)-2,6- |
| | dimethoxybenzyl)azetidine-3-carboxylic acid |
| 246 | 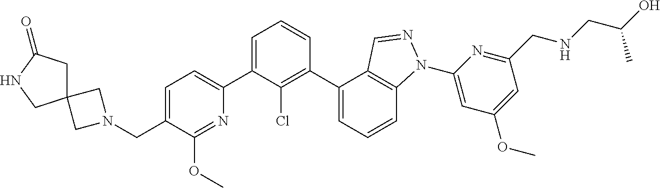 [SMILES: COc1cc(CNC[C@@H](C)O)nc(-n2ncc3c(-c4cccc(-c5ccc(CN6CC7(CNC(=O)C7)C6)c(OC)n5)c4Cl)cccc32)c1] |
| | (R)-2-((6-(2-chloro-3-(1-(6-(((2-hydroxypropyl)amino)methyl)-4- |
| | methoxypyridin-2-yl)-1H-indazol-4-yl)phenyl)-2-methoxypyridin-3- |
| | yl)methyl)-2,6-diazaspiro[3.4]octan-7-one |
| 247 | 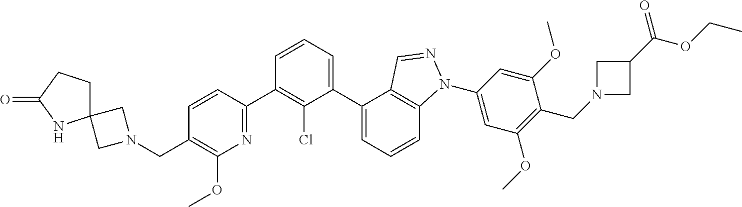 [SMILES: CCOC(=O)C1CN(Cc2c(OC)cc(-n3ncc4c(-c5cccc(-c6ccc(CN7CC8(CCC(=O)N8)C7)c(OC)n6)c5Cl)cccc43)cc2OC)C1] |
| | ethyl 1-(4-(4-(2-chloro-3-(6-methoxy-5-((6-oxo-2,5- |
| | diazaspiro[3.4]octan-2-yl)methyl)pyridin-2-yl)phenyl)-1H-indazol-1- |
| | yl)-2,6-dimethoxybenzyl)azetidine-3-carboxylate |
| 248 | 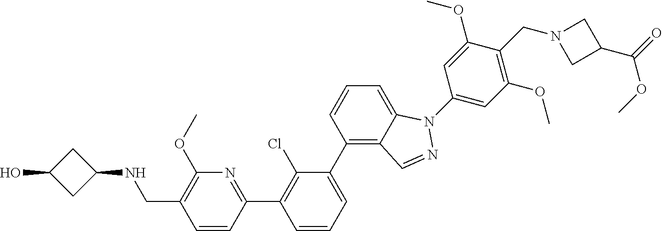 [SMILES: COC(=O)C1CN(Cc2c(OC)cc(-n3ncc4c(-c5cccc(-c6ccc(CN[C@H]7C[C@@H](O)C7)c(OC)n6)c5Cl)cccc43)cc2OC)C1] |
| | methyl 1-(4-(4-(2-chloro-3-(5-((((cis)~3- |
| | hydroxycyclobutyl)amino)methyl)-6-methoxypyridin-2-yl)phenyl)- |
| | 1H-indazol-1-yl)-2,6-dimethoxybenzyl)azetidine-3-carboxylate |
| 249 | 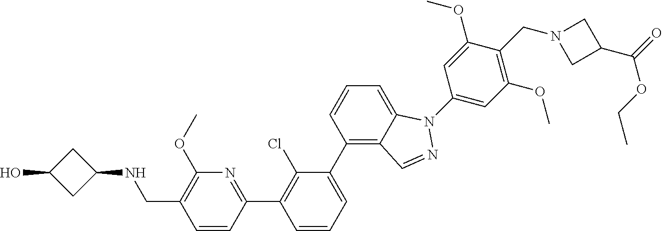 [SMILES: CCOC(=O)C1CN(Cc2c(OC)cc(-n3ncc4c(-c5cccc(-c6ccc(CN[C@H]7C[C@@H](O)C7)c(OC)n6)c5Cl)cccc43)cc2OC)C1] |
| | ethyl 1-(4-(4-(2-chloro-3-(5-((((cis)-3- |
| | hydroxycyclobutyl)amino)methyl)-6-methoxypyridin-2-yl)phenyl)- |
| | 1H-indazol-1-yl)-2,6-dimethoxybenzyl)azetidine-3-carboxylate |
| 250 | 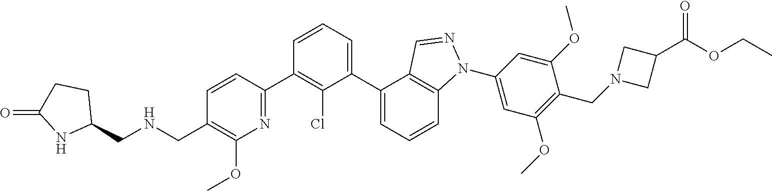 [SMILES: CCOC(=O)C1CN(Cc2c(OC)cc(-n3ncc4c(-c5cccc(-c6ccc(CNC[C@@H]7CCC(=O)N7)c(OC)n6)c5Cl)cccc43)cc2OC)C1] |
| | ethyl (S)-1-(4-(4-(2-chloro-3-(6-methoxy-5-((((5-oxopyrrolidin-2- |
| | yl)methyl)amino)methyl)pyridin-2-yl)phenyl)-1H-indazol-1-yl)-2,6- |
| | dimethoxybenzyl)azetidine-3-carboxylate |
| 251 | 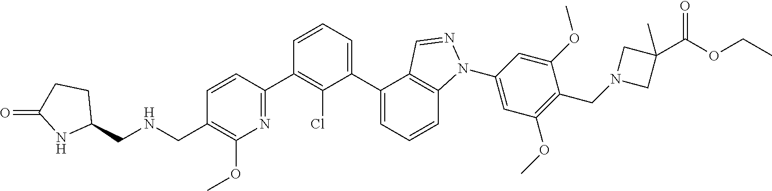 [SMILES: CCOC(=O)C1(C)CN(Cc2c(OC)cc(-n3ncc4c(-c5cccc(-c6ccc(CNC[C@@H]7CCC(=O)N7)c(OC)n6)c5Cl)cccc43)cc2OC)C1] |
| | ethyl (S)-1-(4-(4-(2-chloro-3-(6-methoxy-5-((((5-oxopyrrolidin-2- |
| | yl)methyl)amino)methyl)pyridin-2-yl)phenyl)-1H-indazol-1-yl)-2,6- |
| | dimethoxybenzyl)-3-methylazetidine-3-carboxylate |
| 252 | 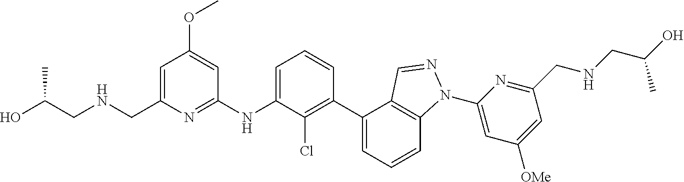 [SMILES: COc1cc(CNC[C@@H](C)O)nc(Nc2cccc(-c3cccc4c3cnn4-c3cc(OC)cc(CNC[C@@H](C)O)n3)c2Cl)c1] |
| | (R)-1-(((6-(4-(2-chloro-3-((6-((((R)-2-hydroxypropyl)amino)methyl)- |
| | 4-methoxypyridin-2-yl)amino)phenyl)-1H-indazol-1-yl)-4- |
| | methoxypyridin-2-yl)methyl)amino)propan-2-ol |
| 253 | 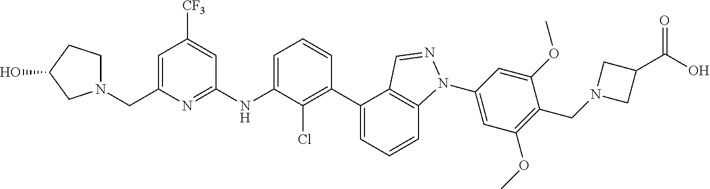 [SMILES: COc1cc(-n2ncc3c(-c4cccc(Nc5cc(C(F)(F)F)cc(CN6CC[C@@H](O)C6)n5)c4Cl)cccc32)cc(OC)c1CN1CC(C(=O)O)C1] |
| | (R)-1-(4-(4-(2-chloro-3-((6-((3-hydroxypyrrolidin-1-yl)methyl)-4- |
| | (trifluoromethyl)pyridin-2-yl)amino)phenyl)-1H-indazol-1-yl)-2,6- |
| | dimethoxybenzyl)azetidine-3-carboxylic acid |
| 254 | 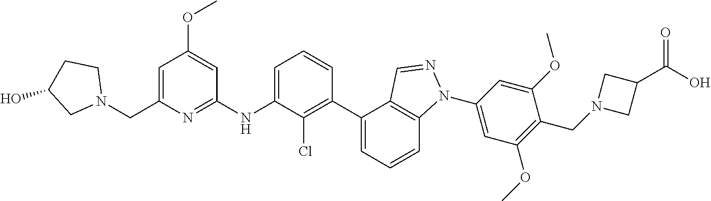 [SMILES: COc1cc(CN2CC[C@@H](O)C2)nc(Nc2cccc(-c3cccc4c3cnn4-c3cc(OC)c(CN4CC(C(=O)O)C4)c(OC)c3)c2Cl)c1] |
| | (R)-1-(4-(4-(2-chloro-3-((6-((3-hydroxypyrrolidin-1-yl)methyl)-4- |
| | methoxypyridin-2-yl)amino)phenyl)-1H-indazol-1-yl)-2,6- |
| | dimethoxybenzyl)azetidine-3-carboxylic acid |
| 255 | 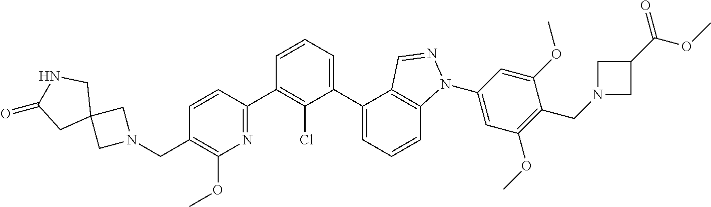 [SMILES: COC(=O)C1CN(Cc2c(OC)cc(-n3ncc4c(-c5cccc(-c6ccc(CN7CC8(CNC(=O)C8)C7)c(OC)n6)c5Cl)cccc43)cc2OC)C1] |
| | methyl 1-(4-(4-(2-chloro-3-(6-methoxy-5-((7-oxo-2,6- |
| | diazaspiro[3.4]octan-2-yl)methyl)pyridin-2-yl)phenyl)-1H-indazol-1- |
| | yl)-2,6-dimethoxybenzyl)azetidine-3-carboxylate |
| 256 | 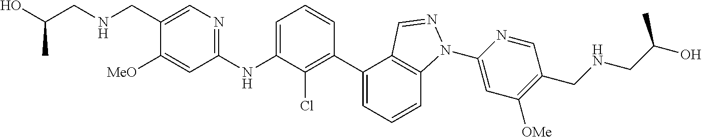 [SMILES: COc1cc(Nc2cccc(-c3cccc4c3cnn4-c3cc(OC)c(CNC[C@@H](C)O)cn3)c2Cl)ncc1CNC[C@@H](C)O] |
| | (R)-1-(((6-(4-(2-chloro-3-((5-((((R)-2-hydroxypropyl)amino)methyl)- |
| | 4-methoxypyridin-2-yl)amino)phenyl)-1H-indazol-1-yl)-4- |
| | methoxypyridin-3-yl)methyl)amino)propan-2-ol |
| 257 | 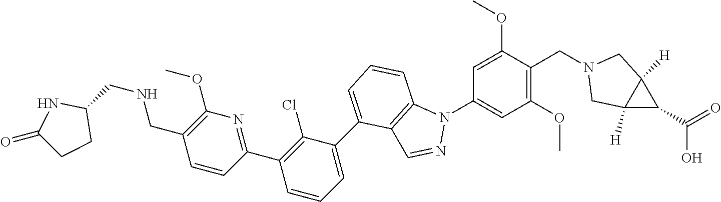 [SMILES: COc1cc(-n2ncc3c(-c4cccc(-c5ccc(CNC[C@@H]6CCC(=O)N6)c(OC)n5)c4Cl)cccc32)cc(OC)c1CN1C[C@@H]2[C@H](C1)[C@H]2C(=O)O] |
| | (1R,5S,6r)-3-(4-(4-(2-chloro-3-(6-methoxy-5-(((((S)-5-oxopyrrolidin- |
| | 2-yl)methyl)amino)methyl)pyridin-2-yl)phenyl)-1H-indazol-1-yl)-2,6- |
| | dimethoxybenzyl)-3-azabicyclo[3.1.0]hexane-6-carboxylic acid |
| 258 | 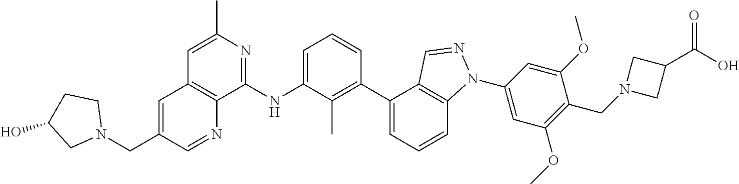 [SMILES: COc1cc(-n2ncc3c(-c4cccc(Nc5nc(C)cc6cc(CN7CC[C@@H](O)C7)cnc56)c4C)cccc32)cc(OC)c1CN1CC(C(=O)O)C1] |
| | (R)-1-(4-(4-(3-((3-((3-hydroxypyrrolidin-1-yl)methyl)-6-methyl-1,7- |
| | naphthyridin-8-yl)amino)-2-methylphenyl)-1H-indazol-1-yl)-2,6- |
| | dimethoxybenzyl)azetidine-3-carboxylic acid |
| 259 | 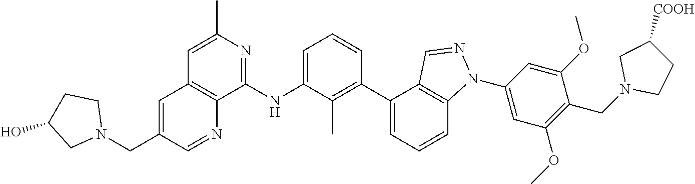 [SMILES: COc1cc(-n2ncc3c(-c4cccc(Nc5nc(C)cc6cc(CN7CC[C@@H](O)C7)cnc56)c4C)cccc32)cc(OC)c1CN1CC[C@@H](C(=O)O)C1] |
| | (R)-1-(4-(4-(3-((3-(((R)-3-hydroxypyrrolidin-1-yl)methyl)-6-methyl- |
| | 1,7-naphthyridin-8-yl)amino)-2-methylphenyl)-1H-indazol-1-yl)-2,6- |
| | dimethoxybenzyl)pyrrolidine-3-carboxylic acid |
| 260 | 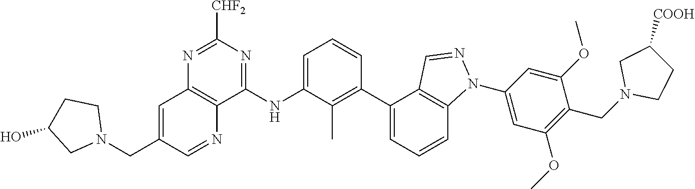 [SMILES: COc1cc(-n2ncc3c(-c4cccc(Nc5nc(C(F)F)nc6cc(CN7CC[C@@H](O)C7)cnc56)c4C)cccc32)cc(OC)c1CN1CC[C@@H](C(=O)O)C1] |
| | (R)-1-(4-(4-(3-((2-(difluoromethyl)-7-(((R)-3-hydroxypyrrolidin-1- |
| | yl)methyl)pyrido[3,2-d]pyrimidin-4-yl)amino)-2-methylphenyl)-1H- |
| | indazol-1-yl)-2,6-dimethoxybenzyl)pyrrolidine-3-carboxylic acid |
| 261 | 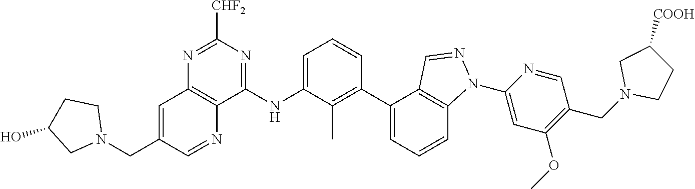 [SMILES: COc1cc(-n2ncc3c(-c4cccc(Nc5nc(C(F)F)nc6cc(CN7CC[C@@H](O)C7)cnc56)c4C)cccc32)ncc1CN1CC[C@@H](C(=O)O)C1] |
| | (R)-1-((6-(4-(3-((2-(difluoromethyl)-7-(((R)-3-hydroxypyrrolidin-1- |
| | yl)methyl)pyrido[3,2-d]pyrimidin-4-yl)amino)-2-methylphenyl)-1H- |
| | indazol-1-yl)-4-methoxypyridin-3-yl)methyl)pyrrolidine-3-carboxylic |
| | acid |
| 262 | 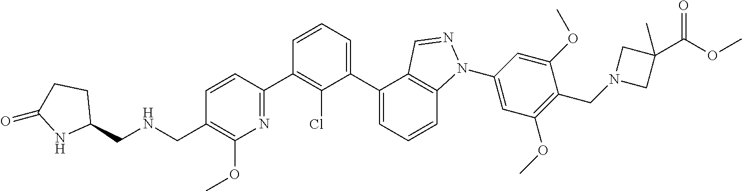 [SMILES: COC(=O)C1(C)CN(Cc2c(OC)cc(-n3ncc4c(-c5cccc(-c6ccc(CNC[C@@H]7CCC(=O)N7)c(OC)n6)c5Cl)cccc43)cc2OC)C1] |
| | methyl (S)-1-(4-(4-(2-chloro-3-(6-methoxy-5-((((5-oxopyrrolidin-2- |
| | yl)methyl)amino)methyl)pyridin-2-yl)phenyl)-1H-indazol-1-yl)-2,6- |
| | dimethoxybenzyl)-3-methylazetidine-3-carboxylate |
| 263 | 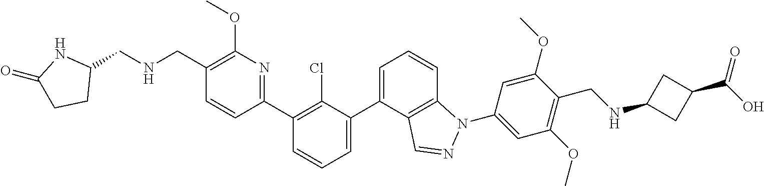 |
| | (cis)-3-((4-(4-(2-chloro-3-(6-methoxy-5-(((((S)-5-oxopyrrolidin-2- |
| | yl)methyl)amino)methyl)pyridin-2-yl)phenyl)-1H-indazol-1-yl)-2,6- |
| | dimethoxybenzyl)amino)cyclobutane-1-carboxylic acid |
| 264 | 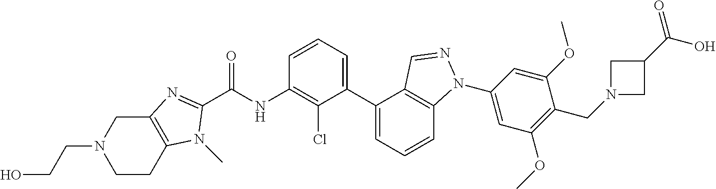 [SMILES: COc1cc(-n2ncc3c(-c4cccc(NC(=O)c5nc6c(n5C)CCN(CCO)C6)c4Cl)cccc32)cc(OC)c1CN1CC(C(=O)O)C1] |
| | 1-(4-(4-(2-chloro-3-(5-(2-hydroxyethyl)-1-methyl-4,5,6,7-tetrahydro- |
| | 1H-imidazo[4,5-c]pyridine-2-carboxamido)phenyl)-1H-indazol-1-yl)- |
| | 2,6-dimethoxybenzyl)azetidine-3-carboxylic acid |
| 265 | 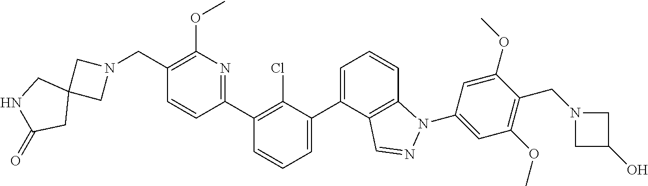 [SMILES: COc1cc(-n2ncc3c(-c4cccc(-c5ccc(CN6CC7(CNC(=O)C7)C6)c(OC)n5)c4Cl)cccc32)cc(OC)c1CN1CC(O)C1] |
| | 2-((6-(2-chloro-3-(1-(4-((3-hydroxyazetidin-1-yl)methyl)-3,5- |
| | dimethoxyphenyl)-1H-indazol-4-yl)phenyl)-2-methoxypyridin-3- |
| | yl)methyl)-2,6-diazaspiro[3.4]octan-7-one |
| 266 | 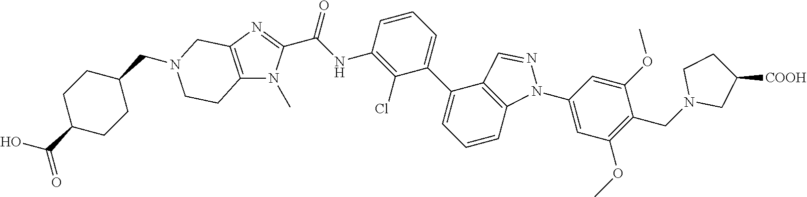 [SMILES: COc1cc(-n2ncc3c(-c4cccc(NC(=O)c5nc6c(n5C)CCN(C[C@H]5CC[C@@H](C(=O)O)CC5)C6)c4Cl)cccc32)cc(OC)c1CN1CC[C@@H](C(=O)O)C1] |
| | (R)-1-(4-(4-(3-(5-((cis-4-carboxycyclohexyl)methyl)-1-methyl- |
| | 4,5,6,7-tetrahydro-1H-imidazo[4,5-c]pyridine-2-carboxamido)-2- |
| | chlorophenyl)-1H-indazol-1-yl)-2,6-dimethoxybenzyl)pyrrolidine-3- |
| | carboxylic acid |
| 267 | 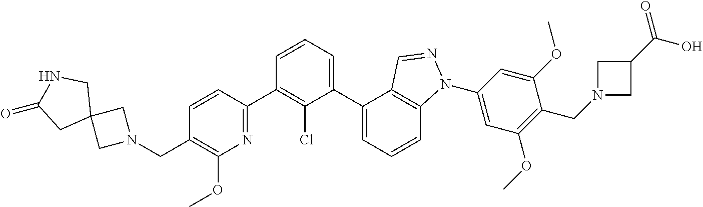 [SMILES: COc1cc(-n2ncc3c(-c4cccc(-c5ccc(CN6CC7(CNC(=O)C7)C6)c(OC)n5)c4Cl)cccc32)cc(OC)c1CN1CC(C(=O)O)C1] |
| | 1-(4-(4-(2-chloro-3-(6-methoxy-5-((7-oxo-2,6-diazaspiro[3.4]octan~2~ |
| | yl)methyl)pyridin-2-yl)phenyl)-1H-indazol-1-yl)-2,6- |
| | dimethoxybenzyl)azetidine-3-carboxylic acid |
| 268 | 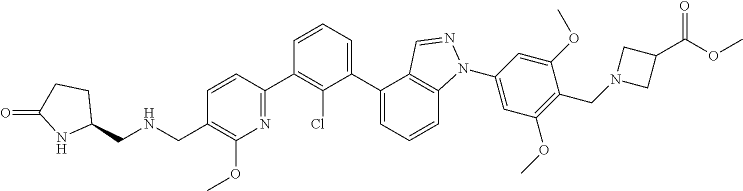 [SMILES: COC(=O)C1CN(Cc2c(OC)cc(-n3ncc4c(-c5cccc(-c6ccc(CNC[C@@H]7CCC(=O)N7)c(OC)n6)c5Cl)cccc43)cc2OC)C1] |
| | methyl (S)-1-(4-(4-(2-chloro-3-(6-methoxy-5-((((5-oxopyrrolidin-2- |
| | yl)methyl)amino)methyl)pyridin-2-yl)phenyl)-1H-indazol-1-yl)-2,6- |
| | dimethoxybenzyl)azetidine-3-carboxylate |
| 269 | 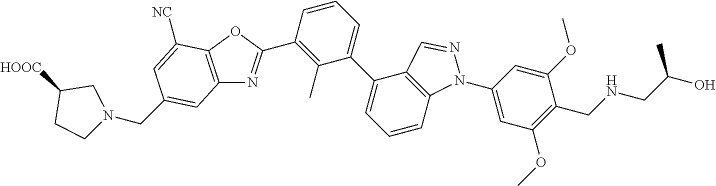 [SMILES: COc1cc(-n2ncc3c(-c4cccc(-c5nc6cc(CN7CC[C@@H](C(=O)O)C7)cc(C#N)c6o5)c4C)cccc32)cc(OC)c1CNC[C@@H](C)O] |
| | (R)-1-((7-cyano-2-(3-(1-(4-((((R)-2-hydroxypropyl)amino)methyl)- |
| | 3,5-dimethoxyphenyl)-1H-indazol-4-yl)-2- |
| | methylphenyl)benzo[d]oxazol-5-yl)methyl)pyrrolidine-3-carboxylic |
| | acid |
| 270 | 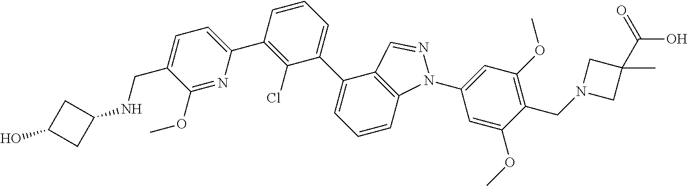 [SMILES: COc1cc(-n2ncc3c(-c4cccc(-c5ccc(CN[C@H]6C[C@@H](O)C6)c(OC)n5)c4Cl)cccc32)cc(OC)c1CN1CC(C)(C(=O)O)C1] |
| | 1-(4-(4-(2-chloro-3-(5-((((cis)-3-hydroxycyclobutyl)amino)methyl)-6- |
| | methoxypyridin-2-yl)phenyl)-1H-indazol-1-yl)-2,6- |
| | dimethoxybenzyl)-3-methylazetidine-3-carboxylic acid |
| 271 | 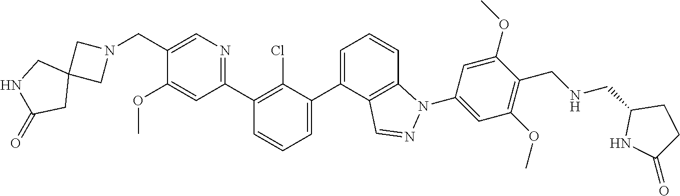 [SMILES: COc1cc(-c2cccc(-c3cccc4c3cnn4-c3cc(OC)c(CNC[C@@H]4CCC(=O)N4)c(OC)c3)c2Cl)ncc1CN1CC2(CNC(=O)C2)C1] |
| | (S)-2-((6-(2-chloro-3-(1-(3,5-dimethoxy-4-((((5-oxopyrrolidin-2- |
| | yl)methyl)amino)methyl)phenyl)-1H-indazol-4-yl)phenyl)-4- |
| | methoxypyridin-3-yl)methyl)-2,6-diazaspiro[3.4]octan-7-one |
| 272 | 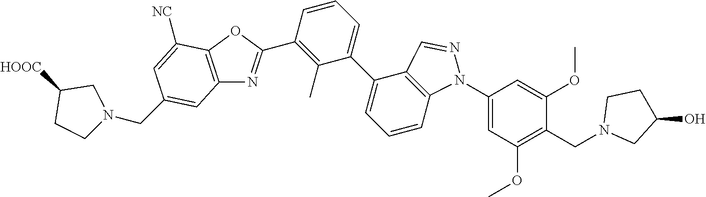 [SMILES: COc1cc(-n2ncc3c(-c4cccc(-c5nc6cc(CN7CC[C@@H](C(=O)O)C7)cc(C#N)c6o5)c4C)cccc32)cc(OC)c1CN1CC[C@@H](O)C1] |
| | (R)-1-((7-cyano-2-(3-(1-(4-(((R)-3-hydroxypyrrolidin-1-yl)methyl)- |
| | 3,5-dimethoxyphenyl)-1H-indazol-4-yl)-2- |
| | methylphenyl)benzo[d]oxazol-5-yl)methyl)pyrrolidine-3-carboxylic |
| | acid |
| 273 | 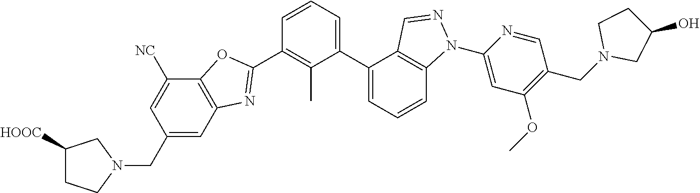 [SMILES: COc1cc(-n2ncc3c(-c4cccc(-c5nc6cc(CN7CC[C@@H](C(=O)O)C7)cc(C#N)c6o5)c4C)cccc32)ncc1CN1CC[C@@H](O)C1] |
| | (R)-1-((7-cyano-2-(3-(1-(5-(((R)-3-hydroxypyrrolidin-1-yl)methyl)-4- |
| | methoxypyridin-2-yl)-1H-indazol-4-yl)-2- |
| | methylphenyl)benzo[d]oxazol-5-yl)methyl)pyrrolidine-3-carboxylic |
| | acid |
| 274 | 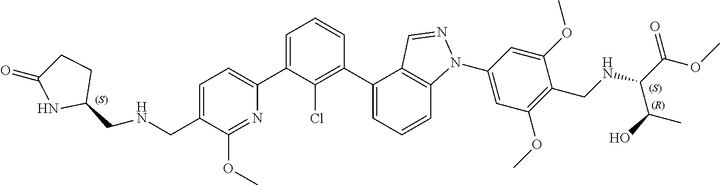 |
| | methyl (4-(4-(2-chloro-3-(6-methoxy-5-(((((S)-5-oxopyrrolidin-2- |
| | yl)methyl)amino)methyl)pyridin-2-yl)phenyl)-1H-indazol-1-yl)-2,6- |
| | dimethoxybenzyl)-L-threoninate |
| 275 | 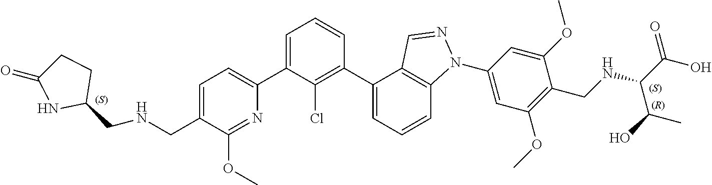 |
| | (4-(4-(2-chloro-3-(6-methoxy-5-(((((S)-5-oxopyrrolidin-2- |
| | yl)methyl)amino)methyl)pyridin-2-yl)phenyl)-1H-indazol-1-yl)-2,6- |
| | dimethoxybenzyl)-L-threonine |
| 276 | 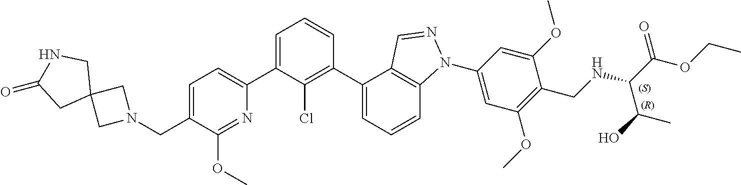 |
| | ethyl (4-(4-(2-chloro-3-(6-methoxy-5-((7-oxo-2,6- |
| | diazaspiro[3.4]octan-2-yl)methyl)pyridin-2-yl)phenyl)-1H-indazol-1- |
| | yl)-2,6-dimethoxybenzyl)-L-threoninate |
| 277 | 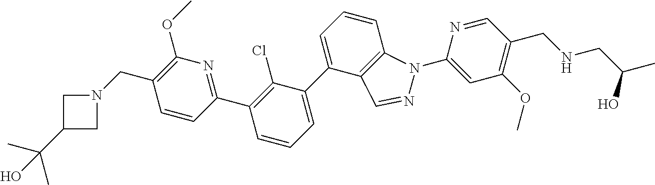 [SMILES: COc1cc(-n2ncc3c(-c4cccc(-c5ccc(CN6CC(C(C)(C)O)C6)c(OC)n5)c4Cl)cccc32)ncc1CNC[C@@H](C)O] |
| | (R)-1-(((6-(4-(2-chloro-3-(5-((3-(2-hydroxypropan-2-yl)azetidin-1- |
| | yl)methyl)-6-methoxypyridin-2-yl)phenyl)-1H-indazol-1-yl)-4- |
| | methoxypyridin-3-yl)methyl)amino)propan-2-ol |
| 278 | 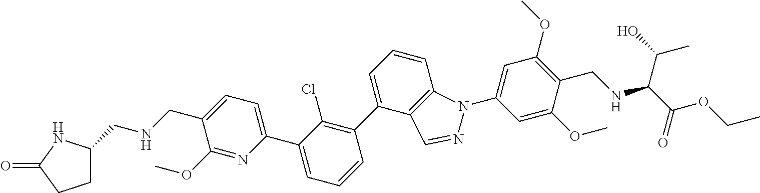 [SMILES: CCOC(=O)[C@@H](NCc1c(OC)cc(-n2ncc3c(-c4cccc(-c5ccc(CNC[C@@H]6CCC(=O)N6)c(OC)n5)c4Cl)cccc32)cc1OC)[C@@H](C)O] |
| | ethyl (4-(4-(2-chloro-3-(6-methoxy-5-(((((S)-5-oxopyrrolidin-2- |
| | yl)methyl)amino)methyl)pyridin-2-yl)phenyl)-1H-indazol-1-yl)-2,6- |
| | dimethoxybenzyl)-L-threoninate |
| 279 | 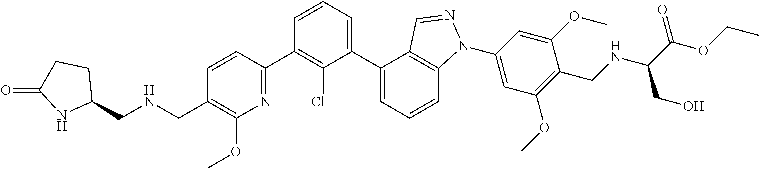 [SMILES: CCOC(=O)[C@@H](CO)NCc1c(OC)cc(-n2ncc3c(-c4cccc(-c5ccc(CNC[C@@H]6CCC(=O)N6)c(OC)n5)c4Cl)cccc32)cc1OC] |
| | ethyl (4-(4-(2-chloro-3-(6-methoxy-5-(((((S)-5-oxopyrrolidin-2- |
| | yl)methyl)amino)methyl)pyridin-2-yl)phenyl)-1H-indazol-1-yl)-2,6- |
| | dimethoxybenzyl)-D-serinate |
| 280 | 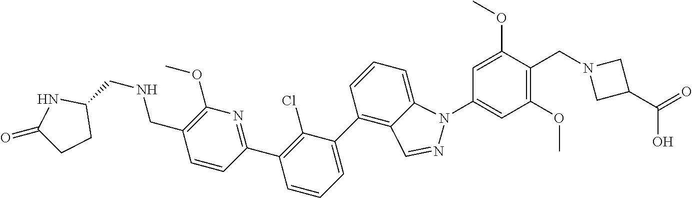 [SMILES: COc1cc(-n2ncc3c(-c4cccc(-c5ccc(CNC[C@@H]6CCC(=O)N6)c(OC)n5)c4Cl)cccc32)cc(OC)c1CN1CC(C(=O)O)C1] |
| | (S)-1-(4-(4-(2-chloro-3-(6-methoxy-5-((((5-oxopyrrolidin-2- |
| | yl)methyl)amino)methyl)pyridin-2-yl)phenyl)-1H-indazol-1-yl)-2,6- |
| | dimethoxybenzyl)azetidine-3-carboxylic acid |
| 281 | 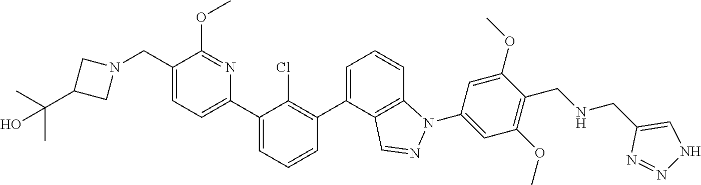 [SMILES: COc1cc(-n2ncc3c(-c4cccc(-c5ccc(CN6CC(C(C)(C)O)C6)c(OC)n5)c4Cl)cccc32)cc(OC)c1CNCc1c[nH]nn1] |
| | 2-(1-((6-(3-(1-(4-((((1H-1,2,3-triazol-4-yl)methyl)amino)methyl)-3,5~ |
| | dimethoxyphenyl)-1H-indazol-4-yl)-2-chlorophenyl)-2- |
| | methoxypyridin-3-yl)methyl)azetidin-3-yl)propan-2-ol |
| 282 | 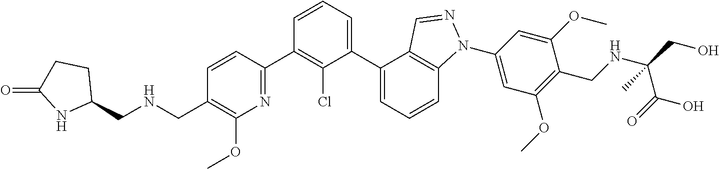 |
| | (S)-2-((4-(4-(2-chloro-3-(6-methoxy-5-(((((S)-5-oxopyrrolidin-2- |
| | yl)methyl)amino)methyl)pyridin-2-yl)phenyl)-1H-indazol-1-yl)-2,6- |
| | dimethoxybenzyl)amino)-3-hydroxy-2-methylpropanoic acid |
| 283 | 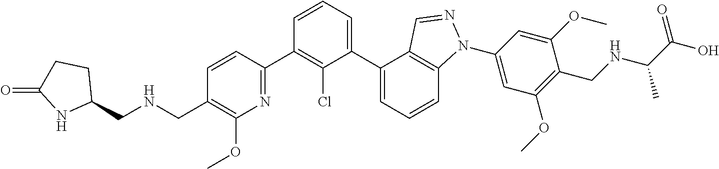 [SMILES: COc1cc(-n2ncc3c(-c4cccc(-c5ccc(CNC[C@@H]6CCC(=O)N6)c(OC)n5)c4Cl)cccc32)cc(OC)c1CN[C@@H](C)C(=O)O] |
| | (4-(4-(2-chloro-3-(6-methoxy-5-(((((S)-5-oxopyrrolidin-2- |
| | yl)methyl)amino)methyl)pyridin-2-yl)phenyl)-1H-indazol-1-yl)-2,6- |
| | dimethoxybenzyl)-L-alanine |
| 284 | 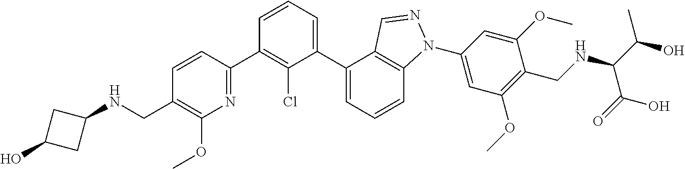 [SMILES: COc1cc(-n2ncc3c(-c4cccc(-c5ccc(CN[C@H]6C[C@@H](O)C6)c(OC)n5)c4Cl)cccc32)cc(OC)c1CN[C@H](C(=O)O)[C@@H](C)O] |
| | (4-(4-(2-chloro-3-(5-((((cis)-3-hydroxycyclobutyl)amino)methyl)-6- |
| | methoxypyridin-2-yl)phenyl)-1H-indazol-1-yl)-2,6- |
| | dimethoxybenzyl)-L-threonine |
| 285 | 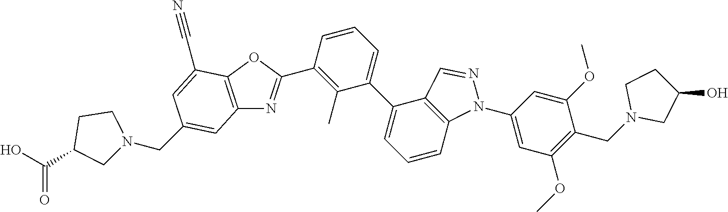 [SMILES: COc1cc(-n2ncc3c(-c4cccc(-c5nc6cc(CN7CC[C@@H](C(=O)O)C7)cc(C#N)c6o5)c4C)cccc32)cc(OC)c1CN1CC[C@@H](O)C1] |
| | (R)-1-((7-cyano-2-(3-(1-(4-(((R)-3-hydroxypyrrolidin-1-yl)methyl)- |
| | 3,5-dimethoxyphenyl)-1H-indazol-4-yl)-2- |
| | methylphenyl)benzo[d]oxazol-5-yl)methyl)pyrrolidine-3-carboxylic |
| | acid |
| 286 | 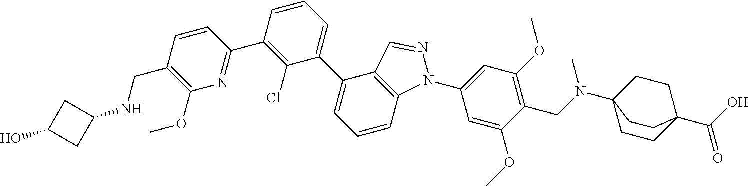 [SMILES: COc1cc(-n2ncc3c(-c4cccc(-c5ccc(CN[C@H]6C[C@@H](O)C6)c(OC)n5)c4Cl)cccc32)cc(OC)c1CN(C)C12CCC(C(=O)O)(CC1)CC2] |
| | 4-((4-(4-(2-chloro-3-(5-((((cis)-3-hydroxycyclobutyl)amino)methyl)- |
| | 6-methoxypyridin-2-yl)phenyl)-1/-indazol-1-yl)-2,6- |
| | dimethoxybenzyl)(methyl)amino)bicyclo[2.2.2]octane-1-carboxylic |
| | acid |
| 287 | 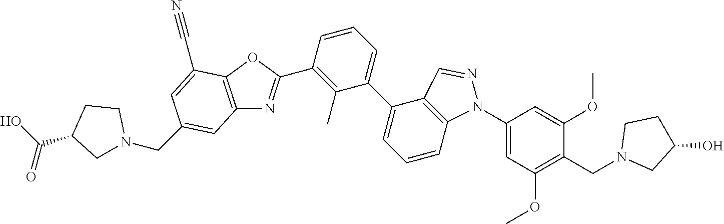 [SMILES: COc1cc(-n2ncc3c(-c4cccc(-c5nc6cc(CN7CC[C@@H](C(=O)O)C7)cc(C#N)c6o5)c4C)cccc32)cc(OC)c1CN1CC[C@H](O)C1] |
| | (R)-1-((7-cyano-2-(3-(1-(4-(((S)-3-hydroxypyrrolidin-1-yl)methyl)- |
| | 3,5-dimethoxyphenyl)-1H-indazol-4-yl)-2- |
| | methylphenyl)benzo[d]oxazol-5-yl)methyl)pyrrolidine-3-carboxylic |
| | acid |
| 288 | 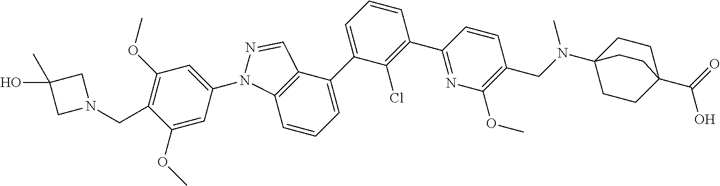 [SMILES: COc1cc(-n2ncc3c(-c4cccc(-c5ccc(CN(C)C67CCC(C(=O)O)(CC6)CC7)c(OC)n5)c4Cl)cccc32)cc(OC)c1CN1CC(C)(O)C1] |
| | 4-(((6-(2-chloro-3-(1-(4-((3-hydroxy-3-methylazetidin-1-yl)methyl)- |
| | 3,5-dimethoxyphenyl)-1H-indazol-4-yl)phenyl)-2-methoxypyridin-3- |
| | yl)methyl)(methyl)amino)bicyclo[2.2.2]octane-1-carboxylic acid |
| 289 | 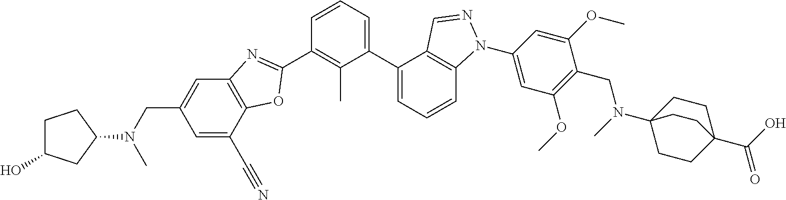 [SMILES: COc1cc(-n2ncc3c(-c4cccc(-c5nc6cc(CN(C)[C@H]7CC[C@@H](O)C7)cc(C#N)c6o5)c4C)cccc32)cc(OC)c1CN(C)C12CCC(C(=O)O)(CC1)CC2] |
| | 4-((4-(4-(3-(7-cyano-5-((((1S,3R)-3- |
| | hydroxycyclopentyl)(methyl)amino)methyl)benzo[d]oxazol-2-yl)-2- |
| | methylphenyl)-1H-indazol-1-yl)-2,6- |
| | dimethoxybenzyl)(methyl)amino)bicyclo[2.2.2]octane-1-carboxylic |
| | acid |
| 290 | 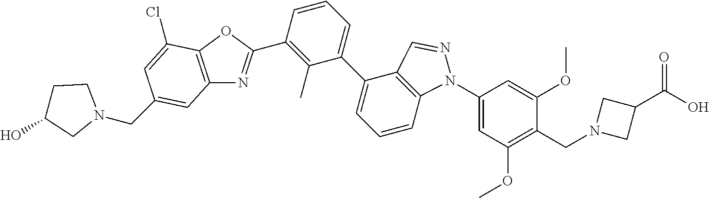 [SMILES: COc1cc(-n2ncc3c(-c4cccc(-c5nc6cc(CN7CC[C@@H](O)C7)cc(Cl)c6o5)c4C)cccc32)cc(OC)c1CN1CC(C(=O)O)C1] |
| | (R)-1-(4-(4-(3-(7-chloro-5-((3-hydroxypyrrolidin-1- |
| | yl)methyl)benzo[d]oxazol-2-yl)-2-methylphenyl)-1H-indazol-1-yl)- |
| | 2,6-dimethoxybenzyl)azetidine-3-carboxylic acid |
| 291 | 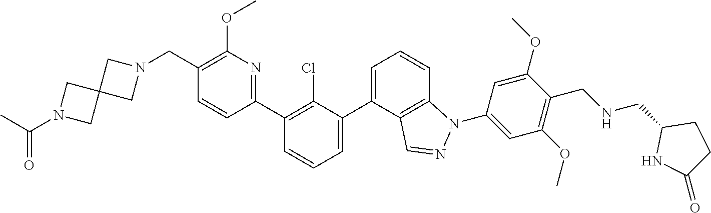 [SMILES: COc1cc(-n2ncc3c(-c4cccc(-c5ccc(CN6CC7(C6)CN(C(C)=O)C7)c(OC)n5)c4Cl)cccc32)cc(OC)c1CNC[C@@H]1CCC(=O)N1] |
| | (S)-5-(((4-(4-(3-(5-((6-acetyl-2,6-diazaspiro[3.3]heptan-2-yl)methyl)- |
| | 6-methoxypyridin-2-yl)-2-chlorophenyl)-1H-indazol-1-yl)-2,6- |
| | dimethoxybenzyl)amino)methyl)pyrrolidin-2-one |
| 292 | 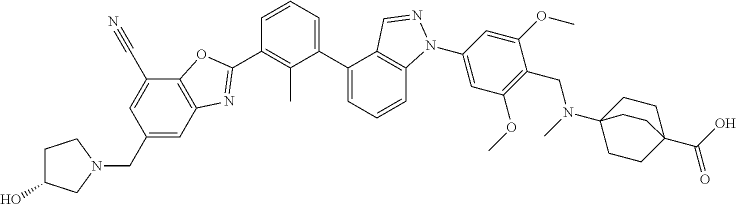 [SMILES: COc1cc(-n2ncc3c(-c4cccc(-c5nc6cc(CN7CC[C@@H](O)C7)cc(C#N)c6o5)c4C)cccc32)cc(OC)c1CN(C)C12CCC(C(=O)O)(CC1)CC2] |
| | (R)-4-((4-(4-(3-(7-cyano-5-((3-hydroxypyrrolidin-1- |
| | yl)methyl)benzo[d]oxazol-2-yl)-2-methylphenyl)-1H-indazol-1-yl)- |
| | 2,6-dimethoxybenzyl)(methyl)amino)bicyclo[2.2.2]octane-1- |
| | carboxylic acid |
| 293 | 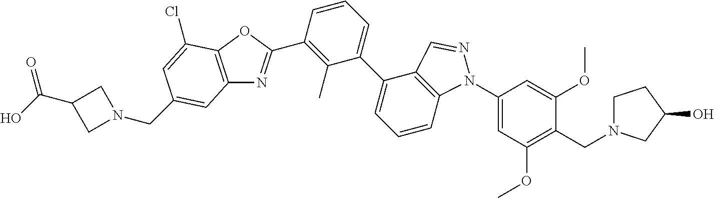 [SMILES: COc1cc(-n2ncc3c(-c4cccc(-c5nc6cc(CN7CC(C(=O)O)C7)cc(Cl)c6o5)c4C)cccc32)cc(OC)c1CN1CC[C@@H](O)C1] |
| | (R)-1-((7-chloro-2-(3-(1-(4-((3-hydroxypyrrolidin-1-yl)methyl)-3,5- |
| | dimethoxyphenyl)-1H-indazol-4-yl)-2-methylphenyl)benzo[d]oxazol- |
| | 5-yl)methyl)azetidine-3-carboxylic acid |
| 294 | 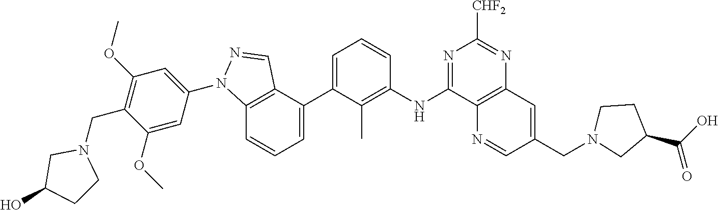 [SMILES: COc1cc(-n2ncc3c(-c4cccc(Nc5nc(C(F)F)nc6cc(CN7CC[C@@H](C(=O)O)C7)cnc56)c4C)cccc32)cc(OC)c1CN1CC[C@@H](O)C1] |
| | (R)-1-((2-(difluoromethyl)-4-((3-(1-(4-(((R)-3-hydroxypyrrolidin-1- |
| | yl)methyl)-3,5-dimethoxyphenyl)-1H-indazol-4-yl)-2- |
| | methylphenyl)amino)pyrido[3,2-d]pyrimidin-7-yl)methyl)pyrrolidine- |
| | 3-carboxylic acid |
| 295 | 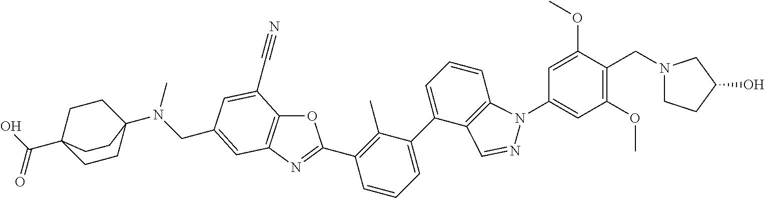 [SMILES: COc1cc(-n2ncc3c(-c4cccc(-c5nc6cc(CN(C)C78CCC(C(=O)O)(CC7)CC8)cc(C#N)c6o5)c4C)cccc32)cc(OC)c1CN1CC[C@@H](O)C1] |
| | (R)-4-(((7-cyano-2-(3-(1-(4-((3-hydroxypyrrolidin-1-yl)methyl)-3,5- |
| | dimethoxyphenyl)-1H-indazol-4-yl)-2-methylphenyl)benzo[d]oxazol- |
| | 5-yl)methyl)(methyl)amino)bicyclo[2.2.2]octane-1-carboxylic acid |
| 296 | 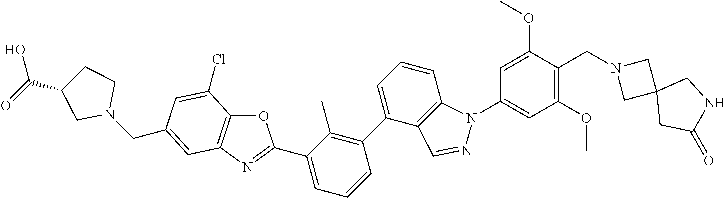 [SMILES: COc1cc(-n2ncc3c(-c4cccc(-c5nc6cc(CN7CC[C@@H](C(=O)O)C7)cc(Cl)c6o5)c4C)cccc32)cc(OC)c1CN1CC2(CNC(=O)C2)C1] |
| | (R)-1-((7-chloro-2-(3-(1-(3,5-dimethoxy-4-((7-oxo-2,6- |
| | diazaspiro[3.4]octan-2-yl)methyl)phenyl)-1H-indazol-4-yl)-2- |
| | methylphenyl)benzo[d]oxazol-5-yl)methyl)pyrrolidine-3-carboxylic |
| | acid |
| 297 | 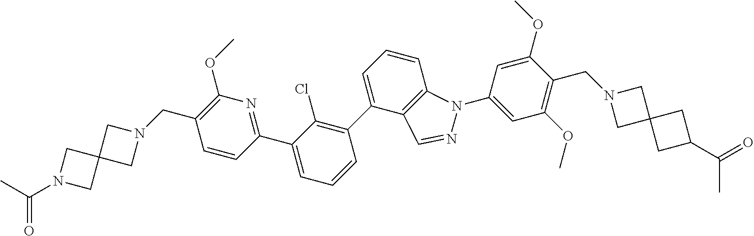 [SMILES: COc1cc(-n2ncc3c(-c4cccc(-c5ccc(CN6CC7(C6)CN(C(C)=O)C7)c(OC)n5)c4Cl)cccc32)cc(OC)c1CN1CC2(CC(C(C)=O)C2)C1] |
| | 1-(6-((6-(3-(1-(4-((6-acetyl-2,6-diazaspiro[3.3]heptan-2-yl)methyl)- |
| | 3,5-dimethoxyphenyl)-1H-indazol-4-yl)-2-chlorophenyl)-2- |
| | methoxypyridin-3-yl)methyl)-2,6-diazaspiro[3.3]heptan-2-yl)ethan-1- |
| | one |
| 298 | 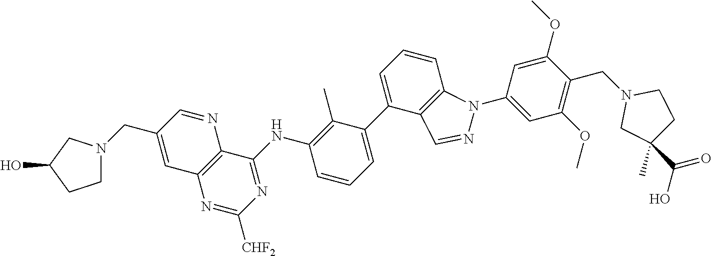 [SMILES: COc1cc(-n2ncc3c(-c4cccc(Nc5nc(C(F)F)nc6cc(CN7CC[C@@H](O)C7)cnc56)c4C)cccc32)cc(OC)c1CN1CC[C@@](C)(C(=O)O)C1] |
| | (R)-1-(4-(4-(3-((2-(difluoromethyl)-7-(((R)-3-hydroxypyrrolidin-1- |
| | yl)methyl)pyrido[3,2-d]pyrimidin-4-yl)amino)-2-methylphenyl)-1H- |
| | indazol-1-yl)-2,6-dimethoxybenzyl)-3-methylpyrrolidine-3-carboxylic |
| | acid |
| 299 | 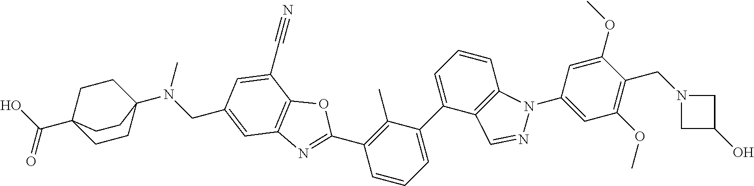 [SMILES: COc1cc(-n2ncc3c(-c4cccc(-c5nc6cc(CN(C)C78CCC(C(=O)O)(CC7)CC8)cc(C#N)c6o5)c4C)cccc32)cc(OC)c1CN1CC(O)C1] |
| | 4-(((7-cyano-2-(3-(1-(4-((3-hydroxyazetidin-1-yl)methyl)-3,5- |
| | dimethoxyphenyl)-1H-indazol-4-yl)-2-methylphenyl)benzo[d]oxazol- |
| | 5-yl)methyl)(methyl)amino)bicyclo[2.2.2]octane-1-carboxylic acid |
| 300 | 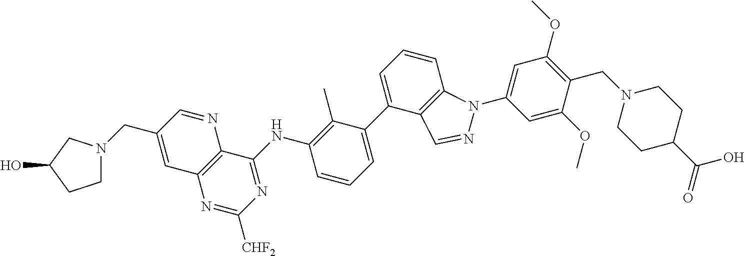 [SMILES: COc1cc(-n2ncc3c(-c4cccc(Nc5nc(C(F)F)nc6cc(CN7CC[C@@H](O)C7)cnc56)c4C)cccc32)cc(OC)c1CN1CCC(C(=O)O)CC1] |
| | (R)-1-(4-(4-(3-((2-(difluoromethyl)-7-((3-hydroxypyrrolidin-1- |
| | yl)methyl)pyrido[3,2-d]pyrimidin-4-yl)amino)-2-methylphenyl)-1H- |
| | indazol-1-yl)-2,6-dimethoxybenzyl)piperidine-4-carboxylic acid |
| 301 | 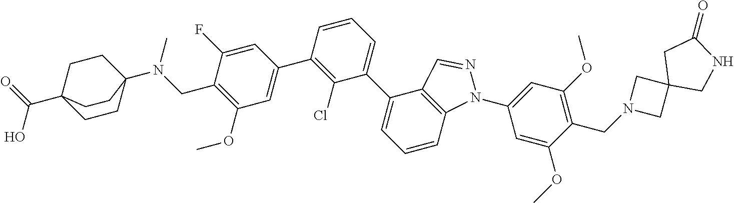 [SMILES: COc1cc(-c2cccc(-c3cccc4c3cnn4-c3cc(OC)c(CN4CC5(CNC(=O)C5)C4)c(OC)c3)c2Cl)cc(F)c1CN(C)C12CCC(C(=O)O)(CC1)CC2] |
| | 4-(((2′-chloro-3′-(1-(3,5-dimethoxy-4-((7-oxo-2,6- |
| | diazaspiro[3.4]octan-2-yl)methyl)phenyl)-1H-indazol-4-yl)-3-fluoro- |
| | 5-methoxy-[1,1′-biphenyl]-4- |
| | yl)methyl)(methyl)amino)bicyclo[2.2.2]octane-1-carboxylic acid |
| 302 | 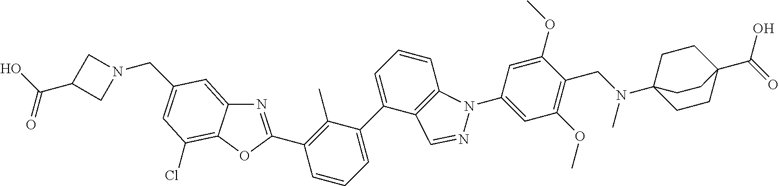 [SMILES: COc1cc(-n2ncc3c(-c4cccc(-c5nc6cc(CN7CC(C(=O)O)C7)cc(Cl)c6o5)c4C)cccc32)cc(OC)c1CN(C)C12CCC(C(=O)O)(CC1)CC2] |
| | 1-((2-(3-(1-(4-(((4-carboxybicyclo[2.2.2]octan-1- |
| | yl)(methyl)amino)methyl)-3,5-dimethoxyphenyl)-1H-indazol-4-yl)-2- |
| | methylphenyl)-7-chlorobenzo[d]oxazol-5-yl)methyl)azetidine-3- |
| | carboxylic acid |
| 303 | 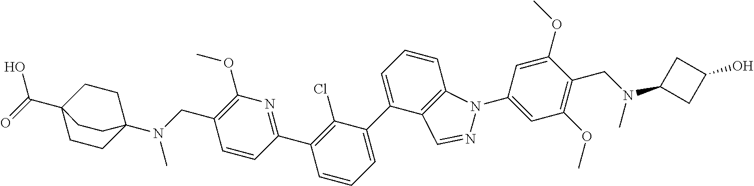 [SMILES: COc1cc(-n2ncc3c(-c4cccc(-c5ccc(CN(C)C67CCC(C(=O)O)(CC6)CC7)c(OC)n5)c4Cl)cccc32)cc(OC)c1CN(C)[C@H]1C[C@H](O)C1] |
| | 4-(((6-(2-chloro-3-(1-(4-((((trans)-3- |
| | hydroxycyclobutyl)(methyl)amino)methyl)-3,5-dimethoxyphenyl)- |
| | 1H-indazol-4-yl)phenyl)-2-methoxypyridin-3- |
| | yl)methyl)(methyl)amino)bicyclo[2.2.2]octane-1-carboxylic acid |
| 304 | 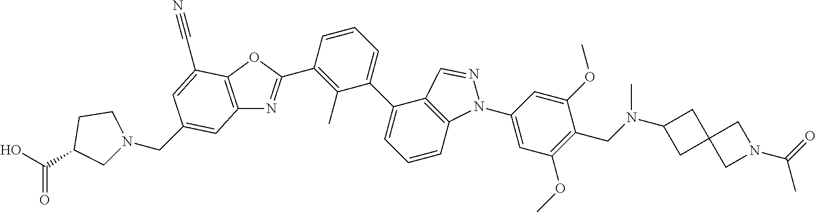 [SMILES: COc1cc(-n2ncc3c(-c4cccc(-c5nc6cc(CN7CC[C@@H](C(=O)O)C7)cc(C#N)c6o5)c4C)cccc32)cc(OC)c1CN(C)C1CC2(C1)CN(C(C)=O)C2] |
| | (R)-1-((2-(3-(1-(4-(((2-acetyl-2-azaspiro[3.3]heptan-6- |
| | yl)(methyl)amino)methyl)-3,5-dimethoxyphenyl)-1H-indazol-4-yl)-2- |
| | methylphenyl)-7-cyanobenzo[d]oxazol-5-yl)methyl)pyrrolidine-3- |
| | carboxylic acid |
| 305 | 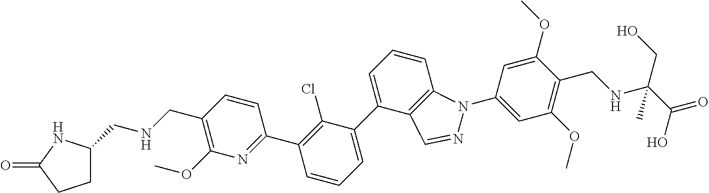 [SMILES: COc1cc(-n2ncc3c(-c4cccc(-c5ccc(CNC[C@@H]6CCC(=O)N6)c(OC)n5)c4Cl)cccc32)cc(OC)c1CN[C@@](C)(CO)C(=O)O] |
| | (S)-2-((4-(4-(2-chloro-3-(6-methoxy-5-(((((S)-5-oxopyrrolidin-2- |
| | yl)methyl)amino)methyl)pyridin-2-yl)phenyl)-1H-indazol-1-yl)-2,6- |
| | dimethoxybenzyl)amino)-3-hydroxy-2-methylpropanoic acid |
| 306 | 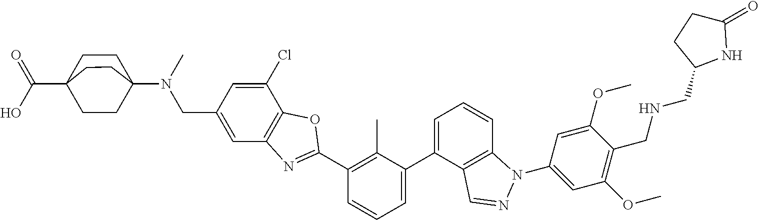 [SMILES: COc1cc(-n2ncc3c(-c4cccc(-c5nc6cc(CN(C)C78CCC(C(=O)O)(CC7)CC8)cc(Cl)c6o5)c4C)cccc32)cc(OC)c1CNC[C@@H]1CCC(=O)N1] |
| | (S)-4-(((7-chloro-2-(3-(1-(3,5-dimethoxy-4-((((5-oxopyrrolidin-2- |
| | yl)methyl)amino)methyl)phenyl)-1H-indazol-4-yl)-2- |
| | methylphenyl)benzo[d]oxazol-5- |
| | yl)methyl)(methyl)amino)bicyclo[2.2.2]octane-1-carboxylic acid |
| 307 | 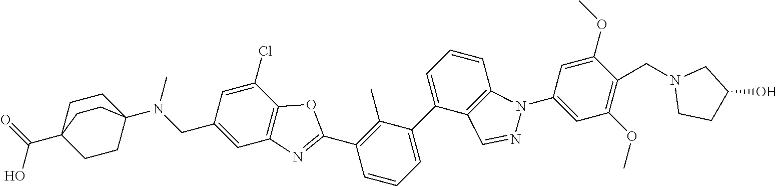 [SMILES: COc1cc(-n2ncc3c(-c4cccc(-c5nc6cc(CN(C)C78CCC(C(=O)O)(CC7)CC8)cc(Cl)c6o5)c4C)cccc32)cc(OC)c1CN1CC[C@@H](O)C1] |
| | (R)-4-(((7-chloro-2-(3-(1-(4-((3-hydroxypyrrolidin-1-yl)methyl)-3,5- |
| | dimethoxyphenyl)-1/-indazol-4-yl)-2-methylphenyl)benzo[d]oxazol- |
| | 5-yl)methyl)(methyl)amino)bicyclo[2.2.2]octane-1-carboxylic acid |
| 308 | 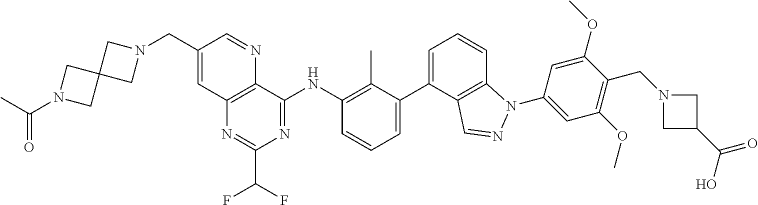 [SMILES: COc1cc(-n2ncc3c(-c4cccc(Nc5nc(C(F)F)nc6cc(CN7CC8(C7)CN(C(C)=O)C8)cnc56)c4C)cccc32)cc(OC)c1CN1CC(C(=O)O)C1] |
| | 1-(4-(4-(3-((7-((6-acetyl-2,6-diazaspiro[3.3]heptan-2-yl)methyl)-2- |
| | (difluoromethyl)pyrido[3,2-d]pyrimidin-4-yl)amino)-2- |
| | methylphenyl)-1H-indazol-1-yl)-2,6-dimethoxybenzyl)azetidine-3- |
| | carboxylic acid |
| 309 | 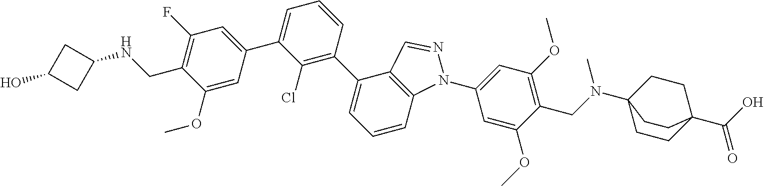 [SMILES: COc1cc(-c2cccc(-c3cccc4c3cnn4-c3cc(OC)c(CN(C)C45CCC(C(=O)O)(CC4)CC5)c(OC)c3)c2Cl)cc(F)c1CN[C@H]1C[C@@H](O)C1] |
| | 4-((4-(4-(2-chloro-3′-fluoro-4′-((((cis)-3- |
| | hydroxycyclobutyl)amino)methyl)-5′-methoxy-[1,1′-biphenyl]-3-yl)- |
| | 1H-indazol-1-yl)-2,6- |
| | dimethoxybenzyl)(methyl)amino)bicyclo[2.2.2]octane-1-carboxylic |
| | acid |
| 310 | 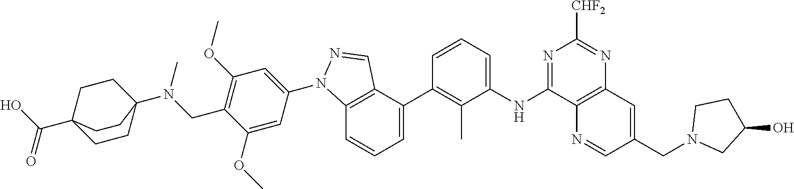 [SMILES: COc1cc(-n2ncc3c(-c4cccc(Nc5nc(C(F)F)nc6cc(CN7CC[C@@H](O)C7)cnc56)c4C)cccc32)cc(OC)c1CN(C)C12CCC(C(=O)O)(CC1)CC2] |
| | (R)-4-((4-(4-(3-((2-(difluoromethyl)-7-((3-hydroxypyrrolidin-1- |
| | yl)methyl)pyrido[3,2-d]pyrimidin-4-yl)amino)-2-methylphenyl)-1H- |
| | indazol-1-yl)-2,6- |
| | dimethoxybenzyl)(methyl)amino)bicyclo[2.2.2]octane-1-carboxylic |
| | acid |
| 311 | 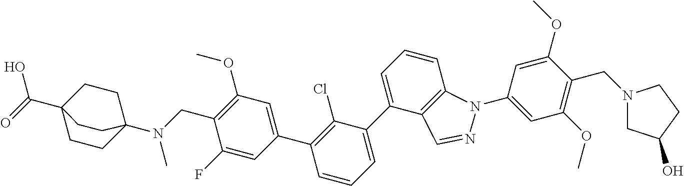 [SMILES: COc1cc(-c2cccc(-c3cccc4c3cnn4-c3cc(OC)c(CN4CC[C@@H](O)C4)c(OC)c3)c2Cl)cc(F)c1CN(C)C12CCC(C(=O)O)(CC1)CC2] |
| | (R)-4-(((2′-chloro-3-fluoro-3′-(1-(4-((3-hydroxypyrrolidin-1- |
| | yl)methyl)-3,5-dimethoxyphenyl)-1H-indazol-4-yl)-5-methoxy-[1,1′- |
| | biphenyl]-4-yl)methyl)(methyl)amino)bicyclo[2.2.2]octane-1- |
| | carboxylic acid |
| 312 | 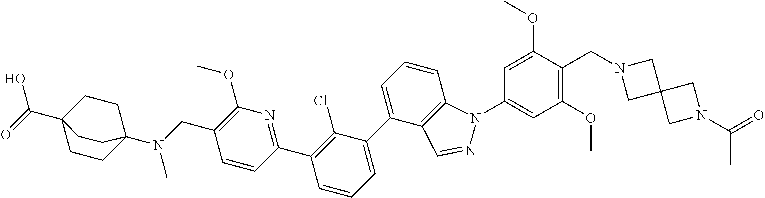 [SMILES: COc1cc(-n2ncc3c(-c4cccc(-c5ccc(CN(C)C67CCC(C(=O)O)(CC6)CC7)c(OC)n5)c4Cl)cccc32)cc(OC)c1CN1CC2(C1)CN(C(C)=O)C2] |
| | 4-(((6-(3-(1-(4-((6-acetyl-2,6-diazaspiro[3.3]heptan-2-yl)methyl)-3,5- |
| | dimethoxyphenyl)-1H-indazol-4-yl)-2-chlorophenyl)-2- |
| | methoxypyridin-3-yl)methyl)(methyl)amino)bicyclo[2.2.2]octane-1- |
| | carboxylic acid |
| 313 | 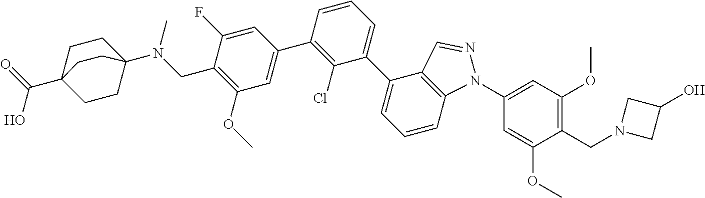 [SMILES: COc1cc(-c2cccc(-c3cccc4c3cnn4-c3cc(OC)c(CN4CC(O)C4)c(OC)c3)c2Cl)cc(F)c1CN(C)C12CCC(C(=O)O)(CC1)CC2] |
| | 4-(((2′-chloro-3-fluoro-3′-(1-(4-((3-hydroxyazetidin-1-yl)methyl)-3,5- |
| | dimethoxyphenyl)-1H-indazol-4-yl)-5-methoxy-[1,1′-bipheny]]-4- |
| | yl)methyl)(methyl)amino)bicyclo[2.2.2]octane-1-carboxylic acid |
| 314 | 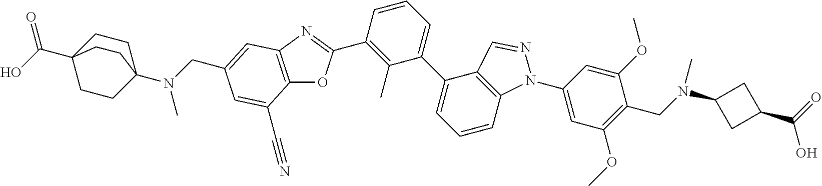 [SMILES: COc1cc(-n2ncc3c(-c4cccc(-c5nc6cc(CN(C)C78CCC(C(=O)O)(CC7)CC8)cc(C#N)c6o5)c4C)cccc32)cc(OC)c1CN(C)[C@H]1C[C@@H](C(=O)O)C1] |
| | 4-(((2-(3-(1-(4-((((cis)-3-carboxycyclobutyl)(methyl)amino)methyl)- |
| | 3,5-dimethoxyphenyl)-1H-indazol-4-yl)-2-methylphenyl)-7- |
| | cyanobenzo[d]oxazol-5- |
| | yl)methyl)(methyl)amino)bicyclo[2.2.2]octane-1-carboxylic acid |
| 315 | 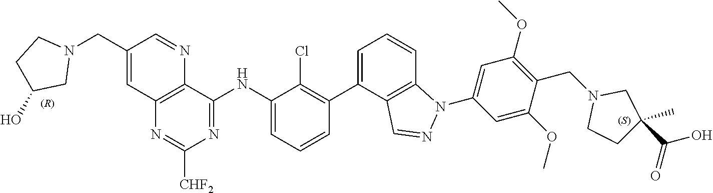 |
| | (S)-1-(4-(4-(2-chloro-3-((2-(difluoromethyl)-7-(((R)-3- |
| | hydroxypyrrolidin-1-yl)methyl)pyrido[3,2-d]pyrimidin-4- |
| | yl)amino)phenyl)-1H-indazol-1-yl)-2,6-dimethoxybenzyl)-3- |
| | methylpyrrolidine-3-carboxylic acid |
| 316 | 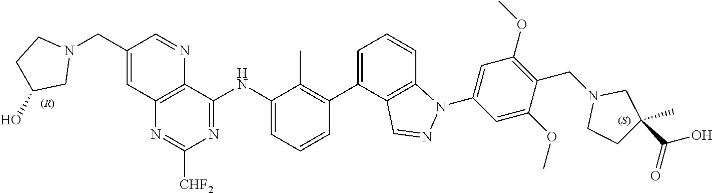 |
| | (S)-1-(4-(4-(3-((2- |
| | (difluoromethyl)-7-(((R)-3-hydroxypyrrolidin-1-yl)methyl)pyrido[3,2- |
| | d]pyrimidin-4-yl)amino)-2-methylphenyl)-1H-indazol-1-yl)-2,6- |
| | dimethoxybenzyl)-3-methylpyrrolidine-3-carboxylic acid |
| 317 | 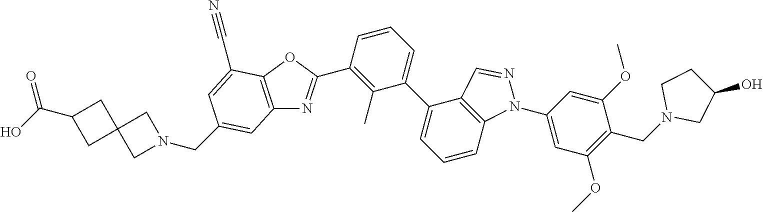 [SMILES: COc1cc(-n2ncc3c(-c4cccc(-c5nc6cc(CN7CC8(CC(C(=O)O)C8)C7)cc(C#N)c6o5)c4C)cccc32)cc(OC)c1CN1CC[C@@H](O)C1] |
| | (R)-2-((7-cyano-2-(3-(1-(4-((3-hydroxypyrrolidin-1-yl)methyl)-3,5- |
| | dimethoxyphenyl)-1H-indazol-4-yl)-2-methylphenyl)benzo[d]oxazol- |
| | 5-yl)methyl)-2-azaspiro[3.3]heptane-6-carboxylic acid |
| 318 | 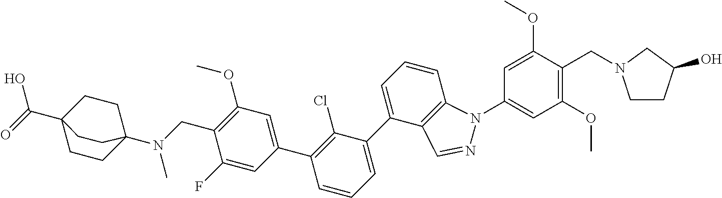 [SMILES: COc1cc(-c2cccc(-c3cccc4c3cnn4-c3cc(OC)c(CN4CC[C@H](O)C4)c(OC)c3)c2Cl)cc(F)c1CN(C)C12CCC(C(=O)O)(CC1)CC2] |
| | (S)-4-(((2′-chloro-3-fluoro-3′-(1-(4-((3-hydroxypyrrolidin-1- |
| | yl)methyl)-3,5-dimethoxyphenyl)-1H-indazol-4-yl)-5-methoxy-[1,l′- |
| | biphenyl]-4-yl)methyl)(methyl)amino)bicyclo[2.2.2]octane-1- |
| | carboxylic acid |
| 319 | 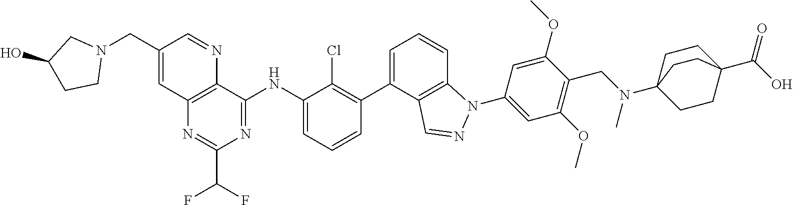 [SMILES: COc1cc(-n2ncc3c(-c4cccc(Nc5nc(C(F)F)nc6cc(CN7CC[C@@H](O)C7)cnc56)c4Cl)cccc32)cc(OC)c1CN(C)C12CCC(C(=O)O)(CC1)CC2] |
| | (R)-4-((4-(4-(2-chloro-3-((2-(difluoromethyl)-7-((3- |
| | hydroxypyrrolidin-1-yl)methyl)pyrido[3,2-d]pyrimidin-4- |
| | yl)amino)phenyl)-1H-indazol-1-yl)-2,6- |
| | dimethoxybenzyl)(methyl)amino)bicyclo[2.2.2]octane-1-carboxylic |
| | acid |
| 320 | 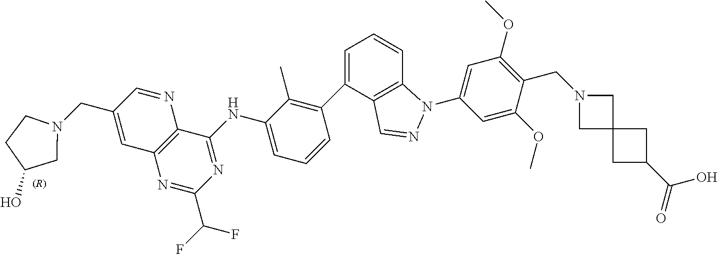 |
| | (R)-2-(4-(4-(3-((2-(difluoromethyl)-7-((3-hydroxypyrrolidin-1- |
| | yl)methyl)pyrido[3,2-d]pyrimidin-4-yl)amino)-2-methylphenyl)-1H- |
| | indazol-1-yl)-2,6-dimethoxybenzyl)-2-azaspiro[3.3]heptane-6- |
| | carboxylic acid |
| 321 | 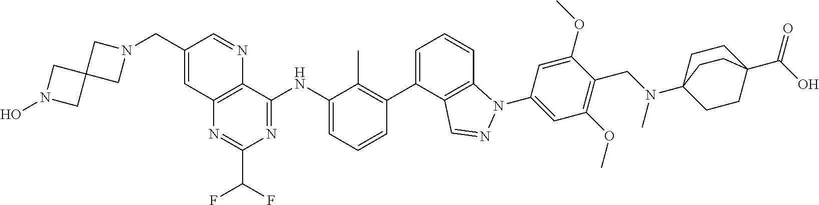 [SMILES: COc1cc(-n2ncc3c(-c4cccc(Nc5nc(C(F)F)nc6cc(CN7CC8(CN(O)C8)C7)cnc56)c4C)cccc32)cc(OC)c1CN(C)C12CCC(C(=O)O)(CC1)CC2] |
| | 4-((4-(4-(3-((2-(difluoromethyl)-7-((6-hydroxy-2-azaspiro[3.3]heptan- |
| | 2-yl)methyl)pyrido[3,2-d]pyrimidin-4-yl)amino)-2-methylphenyl)- |
| | 1H-indazol-1-yl)-2,6- |
| | dimethoxybenzyl)(methyl)amino)bicyclo[2.2.2]octane-1-carboxylic |
| | acid |
| 322 | 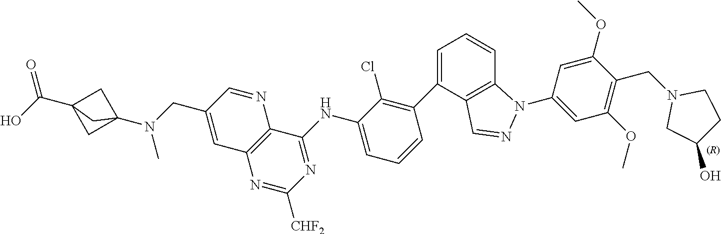 |
| | (R)-3-(((4-((2-chloro-3-(1-(4-((3-hydroxypyrrolidin-1-yl)methyl)-3,5- |
| | dimethoxyphenyl)-1H-indazol-4-yl)phenyl)amino)-2- |
| | (difluoromethyl)pyrido[3,2-d]pyrimidin-7- |
| | yl)methyl)(methyl)amino)bicyclo[1.1.1 ]pentane-1-carboxylic acid |
| 323 | 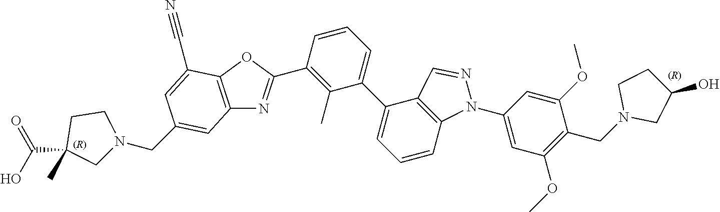 |
| | (R)-1-((7-cyano-2-(3-(1-(4-(((R)-3-hydroxypyrrolidin-1-yl)methyl)- |
| | 3,5-dimethoxyphenyl)-1H-indazol-4-yl)-2- |
| | methylphenyl)benzo[d]oxazol-5-yl)methyl)-3-methylpyrrolidine-3- |
| | carboxylic acid |
| 324 | 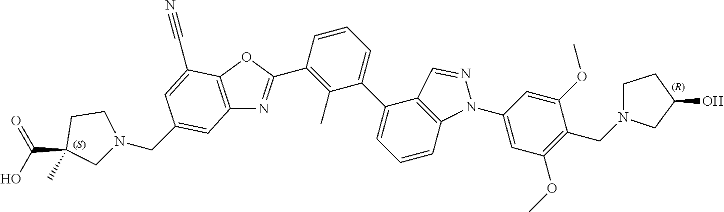 |
| | (S)-1-((7-cyano-2-(3-(1-(4-(((R)-3-hydroxypyrrolidin-1-yl)methyl)- |
| | 3,5-dimethoxyphenyl)-1H-indazol-4-yl)-2- |
| | methylphenyl)benzo[d]oxazol-5-yl)methyl)-3-methylpyrrolidine-3- |
| | carboxylic acid |
| 325 | 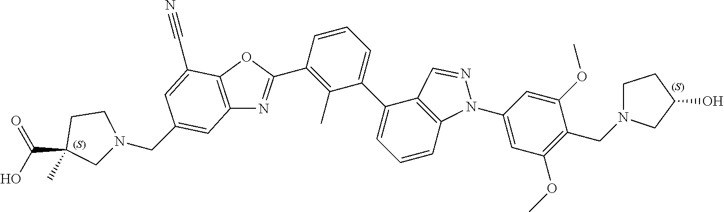 |
| | (S)-1-((7-cyano-2-(3-(1-(4-(((S)-3-hydroxypyrrolidin-1-yl)methyl)- |
| | 3,5-dimethoxyphenyl)-1H-indazol-4-yl)-2- |
| | methylphenyl)benzo[d]oxazol-5-yl)methyl)-3-methylpyrrolidine-3- |
| | carboxylic acid |
| 326 | 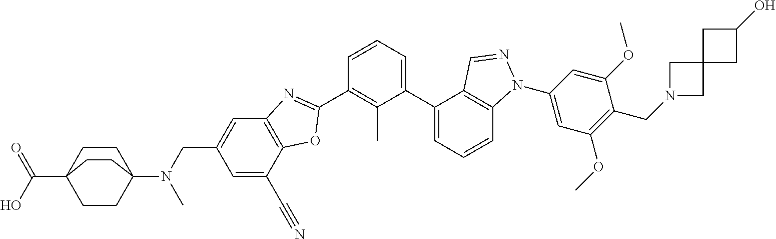 [SMILES: COc1cc(-n2ncc3c(-c4cccc(-c5nc6cc(CN(C)C78CCC(C(=O)O)(CC7)CC8)cc(C#N)c6o5)c4C)cccc32)cc(OC)c1CN1CC2(CC(O)C2)C1] |
| | 4-(((7-cyano-2-(3-(1-(4-((6-hydroxy-2-azaspiro[3.3]heptan-2- |
| | yl)methyl)-3,5-dimethoxyphenyl)-1H-indazol-4-yl)-2- |
| | methylphenyl)benzo[d]oxazol-5- |
| | yl)methyl)(methyl)amino)bicyclo[2.2.2]octane-1-carboxylic acid |
| 327 | 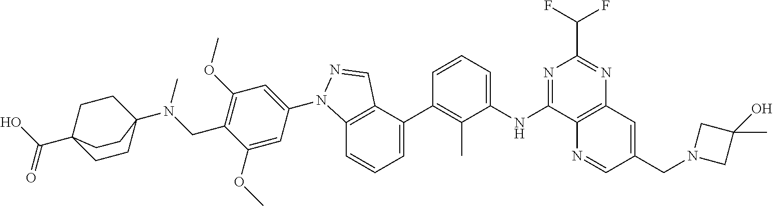 [SMILES: COc1cc(-n2ncc3c(-c4cccc(Nc5nc(C(F)F)nc6cc(CN7CC(C)(O)C7)cnc56)c4C)cccc32)cc(OC)c1CN(C)C12CCC(C(=O)O)(CC1)CC2] |
| | 4-((4-(4-(3-((2-(difluoromethyl)-7-((3-hydroxy-3-methylazetidin-1- |
| | yl)methyl)pyrido[3,2-d]pyrimidin-4-yl)amino)-2-methylphenyl)-1H- |
| | indazol-1-yl)-2,6- |
| | dimethoxybenzyl)(methyl)amino)bicyclo[2.2.2]octane-1-carboxylic |
| | acid |
| 328 | 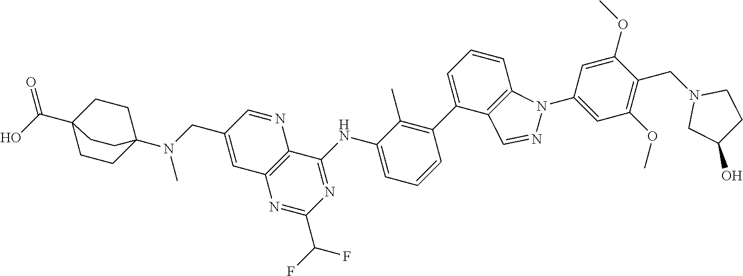 [SMILES: COc1cc(-n2ncc3c(-c4cccc(Nc5nc(C(F)F)nc6cc(CN(C)C78CCC(C(=O)O)(CC7)CC8)cnc56)c4C)cccc32)cc(OC)c1CN1CC[C@@H](O)C1] |
| | (R)-4-(((2-(difluoromethyl)-4-((3-(1-(4-((3-hydroxypyrrolidin-1- |
| | yl)methyl)-3,5-dimethoxyphenyl)-1H-indazol-4-yl)-2- |
| | methylphenyl)amino)pyrido[3,2-d]pyrimidin-7- |
| | yl)methyl)(methyl)amino)bicyclo[2.2.2]octane-1-carboxylic acid |

In some embodiments, the compound is a pharmaceutically acceptable salt of any one of the compounds described in Table 1.

In some aspects, the present disclosure provides a compound being an isotopic derivative (e.g., isotopically labeled compound) of any one of the compounds of the Formulae disclosed herein.

In some embodiments, the compound is an isotopic derivative of any one of the compounds described in Table 1 and prodrugs and pharmaceutically acceptable salts thereof.

In some embodiments, the compound is an isotopic derivative of any one of the compounds described in Table 1 and pharmaceutically acceptable salts thereof.

In some embodiments, the compound is an isotopic derivative of any one of prodrugs of the compounds described in Table 1 and pharmaceutically acceptable salts thereof.

In some embodiments, the compound is an isotopic derivative of any one of the compounds described in Table 1.

It is understood that the isotopic derivative can be prepared using any of a variety of art-recognized techniques. For example, the isotopic derivative can generally be prepared by carrying out the procedures disclosed in the Schemes and/or in the Examples described herein, by substituting an isotopically labeled reagent for a non-isotopically labeled reagent.

In some embodiments, the isotopic derivative is a deuterium labeled compound.

In some embodiments, the isotopic derivative is a deuterium labeled compound of any one of the compounds of the Formulae disclosed herein.

The term “isotopic derivative”, as used herein, refers to a derivative of a compound in which one or more atoms are isotopically enriched or labelled. For example, an isotopic derivative of a compound of Formula (I) is isotopically enriched with regard to, or labelled with, one or more isotopes as compared to the corresponding compound of Formula (I). In some embodiments, the isotopic derivative is enriched with regard to, or labelled with, one or more atoms selected from ²H, ¹³C, ¹⁴C, ¹⁵N, ¹⁸O, ²⁹Si, ³¹P, and ¹⁴S. In some embodiments, the isotopic derivative is a deuterium labeled compound (i.e., being enriched with ²H with regard to one or more atoms thereof). In some embodiments, the compound is a ¹⁸F labeled compound. In some embodiments, the compound is a ¹²³I labeled compound, a ¹²¹I labeled compound, a ¹²⁵I labeled compound, a ¹²⁹I labeled compound, a ¹³¹I labeled compound, a ¹³⁵I labeled compound, or any combination thereof. In some embodiments, the compound is a ³³S labeled compound, a ³⁴S labeled compound, a ³⁵S labeled compound, a ³⁶S labeled compound, or any combination thereof.

It is understood that the ¹⁸F, ¹²³I, ¹²⁴I, ¹²⁵I, ¹²⁹I, ¹³¹I, ¹³⁵I, ³S, ³⁴S, ³⁵S, and/or ³⁶S labeled compound, can be prepared using any of a variety of art-recognized techniques. For example, the deuterium labeled compound can generally be prepared by carrying out the procedures disclosed in the Schemes and/or in the Examples described herein, by substituting a ¹⁸F, ¹²³I, ¹²⁴I, ¹²⁵I, ¹²⁹I, ¹³¹I, ¹³⁵I, ³S, ³⁴S, ³⁵S, and/or ³⁶S labeled reagent for a non-isotope labeled reagent.

A compound of the invention or a pharmaceutically acceptable salt or solvate thereof that contains one or more of the aforementioned ¹⁸F, ¹²³I, ¹²⁴I, ¹²⁵I, ¹²⁹I, ¹³¹I, ¹³⁵I, ³²S, ³⁴S, ³⁵S, and ³⁶S atom(s) is within the scope of the invention. Further, substitution with isotope (e.g., ¹⁸F, ¹²³I, ¹²⁴I, ¹²⁵I, ¹²⁹I, ¹³¹I, ¹³⁵I, ³S, ³⁴S, ³⁵S, and/or ³⁶S) may afford certain therapeutic advantages resulting from greater metabolic stability, e.g., increased in vivo half-life or reduced dosage requirements.

For the avoidance of doubt it is to be understood that, where in this specification a group is qualified by “described herein”, the said group encompasses the first occurring and broadest definition as well as each and all of the particular definitions for that group.

The various functional groups and substituents making up the compounds of the Formula (I) are typically chosen such that the molecular weight of the compound does not exceed 1000 daltons. More usually, the molecular weight of the compound will be less than 900, for example less than 800, or less than 750, or less than 700, or less than 650 daltons. More conveniently, the molecular weight is less than 600 and, for example, is 550 daltons or less.

A suitable pharmaceutically acceptable salt of a compound of the disclosure is, for example, an acid-addition salt of a compound of the disclosure which is sufficiently basic, for example, an acid-addition salt with, for example, an inorganic or organic acid, for example hydrochloric, hydrobromic, sulfuric, phosphoric, trifluoroacetic, formic, citric methane sulfonate or maleic acid. In addition, a suitable pharmaceutically acceptable salt of a compound of the disclosure which is sufficiently acidic is an alkali metal salt, for example a sodium or potassium salt, an alkaline earth metal salt, for example a calcium or magnesium salt, an aminonium salt or a salt with an organic base which affords a pharmaceutically acceptable cation, for example a salt with methylamine, dimethylamine, diethylamine, trimethylamine, piperidine, morpholine or tris-(2-hydroxyethyl)amine.

It will be understood that the compounds of any one of the Formulae disclosed herein and any pharmaceutically acceptable salts thereof, comprise stereoisomers, mixtures of stereoisomers, polymorphs of all isomeric forms of said compounds.

As used herein, the term “isomerism” means compounds that have identical molecular formulae but differ in the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images of each other are termed “enantiomers” or sometimes optical isomers. A mixture containing equal amounts of individual enantiomeric forms of opposite chirality is termed a “racemic mixture.”

As used herein, the term “chiral center” refers to a carbon atom bonded to four nonidentical substituents.

As used herein, the term “chiral isomer” means a compound with at least one chiral center. Compounds with more than one chiral center may exist either as an individual diastereomer or as a mixture of diastereomers, termed “diastereomeric mixture.” When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al., Angew. Chen. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116).

As used herein, the term “geometric isomer” means the diastereomers that owe their existence to hindered rotation about double bonds or a cycloalkyl linker (e.g., 1,3-cyclobutyl).

These configurations are differentiated in their names by the prefixes cis and trans, or Z and E, which indicate that the groups are on the same or opposite side of the double bond in the molecule according to the Cahn-Ingold-Prelog rules.

It is to be understood that the compounds of the present disclosure may be depicted as different chiral isomers or geometric isomers. It is also to be understood that when compounds have chiral isomeric or geometric isomeric forms, all isomeric forms are intended to be included in the scope of the present disclosure, and the naming of the compounds does not exclude any isomeric forms, it being understood that not all isomers may have the same level of activity.

It is to be understood that the structures and other compounds discussed in this disclosure include all atropic isomers thereof. It is also to be understood that not all atropic isomers may have the same level of activity.

As used herein, the term “atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques, it has been possible to separate mixtures of two atropic isomers in select cases.

As used herein, the term “tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solutions where tautomerisation is possible, a chemical equilibrium of the tautomers will be reached. The exact ratio of the tautomers depends on several factors, including temperature, solvent and pH. The concept of tautomers that are interconvertible by tautomerisations is called tautomerism. Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

It is to be understood that the compounds of the present disclosure may be depicted as different tautomers. It should also be understood that when compounds have tautomeric forms, all tautomeric forms are intended to be included in the scope of the present disclosure, and the naming of the compounds does not exclude any tautomer form. It will be understood that certain tautomers may have a higher level of activity than others.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterised by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this disclosure may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the disclosure may have geometric isomeric centers (E- and Z-isomers). It is to be understood that the present disclosure encompasses all optical, diastereoisomers and geometric isomers and mixtures thereof that possess inflammasome inhibitory activity.

The present disclosure also encompasses compounds of the disclosure as defined herein which comprise one or more isotopic substitutions.

It is to be understood that the compounds of any Formula described herein include the compounds themselves, as well as their salts, and their solvates, if applicable. A salt, for example, can be formed between an anion and a positively charged group (e.g., amino) on a substituted compound disclosed herein. Suitable anions include chloride, bromide, iodide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate, and acetate (e.g., trifluoroacetate).

As used herein, the term “pharmaceutically acceptable anion” refers to an anion suitable for forming a pharmaceutically acceptable salt. Likewise, a salt can also be formed between a cation and a negatively charged group (e.g., carboxylate) on a substituted compound disclosed herein. Suitable cations include sodium ion, potassium ion, magnesium ion, calcium ion, and an aminonium cation such as tetramethylaminonium ion or diethylamine ion. The substituted compounds disclosed herein also include those salts containing quaternary nitrogen atoms.

It is to be understood that the compounds of the present disclosure, for example, the salts of the compounds, can exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.

As used herein, the term “solvate” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O.

As used herein, the term “analog” refers to a chemical compound that is structurally similar to another but differs slightly in composition (as in the replacement of one atom by an atom of a different element or in the presence of a particular functional group, or the replacement of one functional group by another functional group). Thus, an analog is a compound that is similar or comparable in function and appearance, but not in structure or origin to the reference compound.

As used herein, the term “derivative” refers to compounds that have a common core structure and are substituted with various groups as described herein.

As used herein, the term “bioisostere” refers to a compound resulting from the exchange of an atom or of a group of atoms with another, broadly similar, atom or group of atoms. The objective of a bioisosteric replacement is to create a new compound with similar biological properties to the parent compound. The bioisosteric replacement may be physicochemically or topologically based. Examples of carboxylic acid bioisosteres include, but are not limited to, acyl sulfonamides, tetrazoles, sulfonates and phosphonates. See, e.g., Patani and LaVoie, Chem. Rev. 96, 3147-3176, 1996.

It is also to be understood that certain compounds of any one of the Formulae disclosed herein may exist in solvated as well as unsolvated forms such as, for example, hydrated forms. A suitable pharmaceutically acceptable solvate is, for example, a hydrate such as hemi-hydrate, a mono-hydrate, a di-hydrate or a tri-hydrate. It is to be understood that the disclosure encompasses all such solvated forms that possess inflammasome inhibitory activity.

It is also to be understood that certain compounds of any one of the Formulae disclosed herein may exhibit polymorphism, and that the disclosure encompasses all such forms, or mixtures thereof, which possess inflammasome inhibitory activity. It is generally known that crystalline materials may be analysed using conventional techniques such as X-Ray Powder Diffraction analysis, Differential Scanning Calorimetry, Thermal Gravimetric Analysis, Diffuse Reflectance Infrared Fourier Transform (DRIFT) spectroscopy, Near Infrared (NIR) spectroscopy, solution and/or solid state nuclear magnetic resonance spectroscopy. The water content of such crystalline materials may be determined by Karl Fischer analysis.

Compounds of any one of the Formulae disclosed herein may exist in a number of different tautomeric forms and references to compounds of Formula (I) include all such forms. For the avoidance of doubt, where a compound can exist in one of several tautomeric forms, and only one is specifically described or shown, all others are nevertheless embraced by Formula (I). Examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

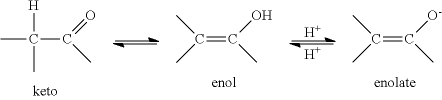

Compounds of any one of the Formulae disclosed herein containing an amine function may also form N-oxides. A reference herein to a compound of Formula (I) that contains an amine function also includes the N-oxide. Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle. N-oxides can be formed by treatment of the corresponding amine with an oxidizing agent such as hydrogen peroxide or a peracid (e.g. a peroxycarboxylic acid), see for example Advanced Organic Chemistry, by Jerry March, 4th Edition, Wiley Interscience, pages. More particularly, N-oxides can be made by the procedure of L. W. Deady (Syn. Comm. 1977, 7, 509-514) in which the amine compound is reacted with meta-chloroperoxybenzoic acid (mCPBA), for example, in an inert solvent such as dichloromethane.

The compounds of any one of the Formulae disclosed herein may be administered in the form of a prodrug which is broken down in the human or animal body to release a compound of the disclosure. A prodrug may be used to alter the physical properties and/or the pharmacokinetic properties of a compound of the disclosure. A prodrug can be formed when the compound of the disclosure contains a suitable group or substituent to which a property-modifying group can be attached. Examples of prodrugs include derivatives containing in vivo cleavable alkyl or acyl substituents at the ester or amide group in any one of the Formulae disclosed herein.

Accordingly, the present disclosure includes those compounds of any one of the Formulae disclosed herein as defined hereinbefore when made available by organic synthesis and when made available within the human or animal body by way of cleavage of a prodrug thereof. Accordingly, the present disclosure includes those compounds of any one of the Formulae disclosed herein that are produced by organic synthetic means and also such compounds that are produced in the human or animal body by way of metabolism of a precursor compound, that is a compound of any one of the Formulae disclosed herein may be a synthetically-produced compound or a metabolically-produced compound.

A suitable pharmaceutically acceptable prodrug of a compound of any one of the Formulae disclosed herein is one that is based on reasonable medical judgment as being suitable for administration to the human or animal body without undesirable pharmacological activities and without undue toxicity. Various forms of prodrug have been described, for example in the following documents: a) Methods in Enzymology, Vol. 42, p. 309-396, edited by K. Widder, et al. (Academic Press, 1985); b) Design of Pro-drugs, edited by H. Bundgaard, (Elsevier, 1985); c) A Textbook of Drug Design and Development, edited by Krogsgaard-Larsen and H. Bundgaard, Chapter 5 “Design and Application of Pro-drugs”, by H. Bundgaard p. 113-191 (1991); d) H. Bundgaard, Advanced Drug Delivery Reviews, 8, 1-38 (1992); e) H. Bundgaard, et al., Journal of Pharmaceutical Sciences, 77, 285 (1988); f) N. Kakeya, et al., Chem. Pharm. Bull., 32, 692 (1984); g) T. Higuchi and V. Stella, “Pro-Drugs as Novel Delivery Systems”, A.C.S. Symposium Series, Volume 14; and h) E. Roche (editor), “Bioreversible Carriers in Drug Design”, Pergamon Press, 1987.

A suitable pharmaceutically acceptable prodrug of a compound of any one of the Formulae disclosed herein that possesses a hydroxy group is, for example, an in vivo cleavable ester or ether thereof. An in vivo cleavable ester or ether of a compound of any one of the Formulae disclosed herein containing a hydroxy group is, for example, a pharmaceutically acceptable ester or ether which is cleaved in the human or animal body to produce the parent hydroxy compound. Suitable pharmaceutically acceptable ester forming groups for a hydroxy group include inorganic esters such as phosphate esters (including phosphoramidic cyclic esters). Further suitable pharmaceutically acceptable ester forming groups for a hydroxy group include C₁-C₁₀ alkanoyl groups such as acetyl, benzoyl, phenylacetyl and substituted benzoyl and phenylacetyl groups, C₁-C₁₀ alkoxycarbonyl groups such as ethoxycarbonyl, N,N—(C₁-C₆ alkyl)₂ carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁-C₄ alkyl)piperazin-1-ylmethyl. Suitable pharmaceutically acceptable ether forming groups for a hydroxy group include α-acyloxyalkyl groups such as acetoxymethyl and pivaloyloxymethyl groups.

A suitable pharmaceutically acceptable prodrug of a compound of any one of the Formulae disclosed herein that possesses a carboxy group is, for example, an in vivo cleavable amide thereof, for example an amide formed with an amine such as aminonia, a C₁₋₄alkylamine such as methylamine, a (C₁-C₄ alkyl)₂ amine such as dimethylamine, N-ethyl-N-methylamine or diethylamine, a C₁-C₄ alkoxy-C₂-C₄ alkylamine such as 2-methoxyethylamine, a phenyl-C₁-C₄ alkylamine such as benzylamine and amino acids such as glycine or an ester thereof.

A suitable pharmaceutically acceptable prodrug of a compound of any one of the Formulae disclosed herein that possesses an amino group is, for example, an in vivo cleavable amide derivative thereof. Suitable pharmaceutically acceptable amides from an amino group include, for example an amide formed with C₁-C₁₀ alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl,morpholinomethyl,piperazin-1-ylmethyl and 4-(C₁-C₄ alkyl)piperazin-1-ylmethyl.

The in vivo effects of a compound of any one of the Formulae disclosed herein may be exerted in part by one or more metabolites that are formed within the human or animal body after administration of a compound of any one of the Formulae disclosed herein. As stated hereinbefore, the in vivo effects of a compound of any one of the Formulae disclosed herein may also be exerted by way of metabolism of a precursor compound (a prodrug).

Suitably, the present disclosure excludes any individual compounds not possessing the biological activity defined herein.

### Methods of Synthesis

In some aspects, the present disclosure provides a method of preparing a compound of the present disclosure.

In some aspects, the present disclosure provides a method of a compound, comprising one or more steps as described herein.

In some aspects, the present disclosure provides a compound obtainable by, or obtained by, or directly obtained by a method for preparing a compound as described herein.

In some aspects, the present disclosure provides an intermediate as described herein, being suitable for use in a method for preparing a compound as described herein.

The compounds of the present disclosure can be prepared by any suitable technique known in the art. Particular processes for the preparation of these compounds are described further in the accompanying examples.

In the description of the synthetic methods described herein and in any referenced synthetic methods that are used to prepare the starting materials, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions utilized.

It will be appreciated that during the synthesis of the compounds of the disclosure in the processes defined herein, or during the synthesis of certain starting materials, it may be desirable to protect certain substituent groups to prevent their undesired reaction. The skilled chemist will appreciate when such protection is required, and how such protecting groups may be put in place, and later removed. For examples of protecting groups see one of the many general texts on the subject, for example, ‘Protective Groups in Organic Synthesis’ by Theodora Green (publisher. John Wiley & Sons). Protecting groups may be removed by any convenient method described in the literature or known to the skilled chemist as appropriate for the removal of the protecting group in question, such methods being chosen so as to effect removal of the protecting group with the minimum disturbance of groups elsewhere in the molecule. Thus, if reactants include, for example, groups such as amino, carboxy or hydroxy it may be desirable to protect the group in some of the reactions mentioned herein.

By way of example, a suitable protecting group for an amino or alkylamino group is, for example, an acyl group, for example an alkanoyl group such as acetyl, an alkoxycarbonyl group, for example a methoxycarbonyl, ethoxycarbonyl, or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed by, for example, hydrolysis with a suitable base such as an alkali metal hydroxide, for example lithium or sodium hydroxide. Alternatively an acyl group such as a tert-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium on carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium, sodium hydroxide or aminonia. Alternatively an arylmethyl group such as a benzyl group may be removed, for example, by hydrogenation over a catalyst such as palladium on carbon.

A suitable protecting group for a carboxy group is, for example, an esterifying group, for example a methyl or an ethyl group which may be removed, for example, by hydrolysis with a base such as sodium hydroxide, or for example a tert-butyl group which may be removed, for example, by treatment with an acid, for example an organic acid such as trifluoroacetic acid, or for example a benzyl group which may be removed, for example, by hydrogenation over a catalyst such as palladium on carbon.

Once a compound of Formula (I) has been synthesized by any one of the processes defined herein, the processes may then further comprise the additional steps of; (i) removing any protecting groups present; (ii) converting the compound Formula (I) into another compound of Formula (I); (iii) forming a pharmaceutically acceptable salt, hydrate or solvate thereof; and/or (iv) forming a prodrug thereof.

The resultant compounds of Formula (I) can be isolated and purified using techniques well known in the art.

Conveniently, the reaction of the compounds is carried out in the presence of a suitable solvent, which is preferably inert under the respective reaction conditions. Examples of suitable solvents comprise but are not limited to hydrocarbons, such as hexane, petroleum ether, benzene, toluene or xylene; chlorinated hydrocarbons, such as trichlorethylene, 1,2-dichloroethane, tetrachloromethane, chloroform or dichloromethane; alcohols, such as methanol, ethanol, isopropanol, n-propanol, n-butanol or tert-butanol; ethers, such as diethyl ether, diisopropyl ether, tetrahydrofuran (THF), 2-methyltetrahydrofuran, cyclopentylmethyl ether (CPME), methyl tert-butyl ether (MTBE) or dioxane; glycol ethers, such as ethylene glycol monomethyl or monoethyl ether or ethylene glycol dimethyl ether (diglyme); ketones, such as acetone, methylisobutylketone (MIBK) or butanone; amides, such as acetamide, dimethylacetamide, dimethylformamide (DMF) or N-methylpyrrolidinone (NMP); nitriles, such as acetonitrile; sulfoxides, such as dimethyl sulfoxide (DMSO); nitro compounds, such as nitromethane or nitrobenzene; esters, such as ethyl acetate or methyl acetate, or mixtures of the said solvents or mixtures with water.

The reaction temperature is suitably between about −100° C. and 300° C., depending on the reaction step and the conditions used.

Reaction times are generally in the range between a fraction of a minute and several days, depending on the reactivity of the respective compounds and the respective reaction conditions.

Suitable reaction times are readily determinable by methods known in the art, for example reaction monitoring. Based on the reaction temperatures given above, suitable reaction times generally lie in the range between 10 minutes and 48 hours.

Moreover, by utilizing the procedures described herein, in conjunction with ordinary skills in the art, additional compounds of the present disclosure can be readily prepared. Those skilled in the art will readily understand that known variations of the conditions and processes of the following preparative procedures can be used to prepare these compounds.

As will be understood by the person skilled in the art of organic synthesis, compounds of the present disclosure are readily accessible by various synthetic routes, some of which are exemplified in the accompanying examples. The skilled person will easily recognise which kind of reagents and reactions conditions are to be used and how they are to be applied and adapted in any particular instance—wherever necessary or useful—in order to obtain the compounds of the present disclosure. Furthermore, some of the compounds of the present disclosure can readily be synthesized by reacting other compounds of the present disclosure under suitable conditions, for instance, by converting one particular functional group being present in a compound of the present disclosure, or a suitable precursor molecule thereof, into another one by applying standard synthetic methods, like reduction, oxidation, addition or substitution reactions; those methods are well known to the skilled person. Likewise, the skilled person will apply—whenever necessary or useful—synthetic protecting (or protective) groups; suitable protecting groups as well as methods for introducing and removing them are well-known to the person skilled in the art of chemical synthesis and are described, in more detail, in, e.g., P. G. M. Wuts, T. W. Greene, “Greene's Protective Groups in Organic Synthesis”, 4th edition (2006) (John Wiley & Sons).

General routes for the preparation of a compound of the application are described in Schemes 1-6 herein.

The compounds of the present disclosure may be synthesized by known organic synthesis reactions according to any of the synthetic routes in Schemes 1-6. Moreover, the starting materials, reagents, catalysts, and solvents for the Schemes of the instant disclosure are commonly known compounds or can be prepared by known procedures. Additionally, further functionalization (e.g. esterification, acetylation, or alkylation) may be implemented with known reaction conditions.

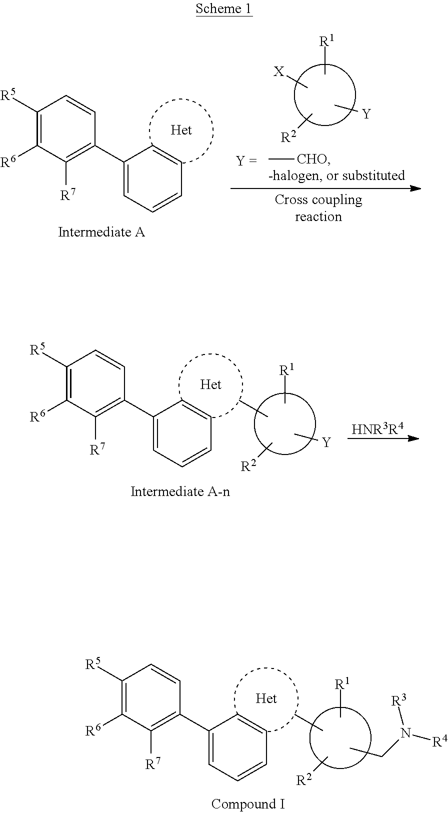

Compound I may be prepared according to Scheme 1. Biaryl Intermediate A may react with aryl halides (X=Cl, Br, or I) or boronates (X=B(OR)₂) under cross coupling conditions (e.g., in the presence of a metal catalyst and a suitable base) in an appropriate solvent (e.g., toluene or DMSO) or by an S_{N}Ar reaction to provide the triaryl Intermediate A-n. Reductive amination of aldehyde Intermediate A-n and amines of formula HNR³R⁴ may occur in a mixture of MeOH and DCM by using a reducing reagent (e.g., sodium cyanoborohydride) to prepare Compound 1. The addition of amine HNR³R⁴ may occur by using other appropriate methods. The remaining protecting group may be removed with known deprotection methods. Compound I may be purified by column chromatography (e.g., using silica gel (230-400 mesh) and/or Sephadex LH-20).

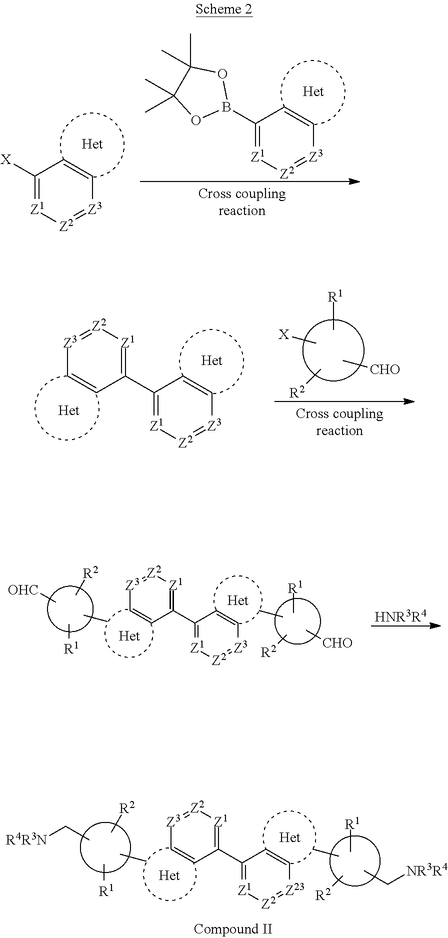

Compound H may be prepared according to Scheme 2. Suzuki cross coupling of biaryl halide (X=Cl, Br, or I) and biaryl boronate in a mixture of 1,4-dioxane and H₂O may result in dimeric intermediates which may undergo reductive amination with amines of formula HNR³R⁴ in a mixture of MeOH and DCM to provide Compound H. The remaining protecting group may be removed with known deprotection methods. Compound II may be purified by column chromatography (e.g., using silica gel (230-400 mesh) and/or Sephadex LH-20).

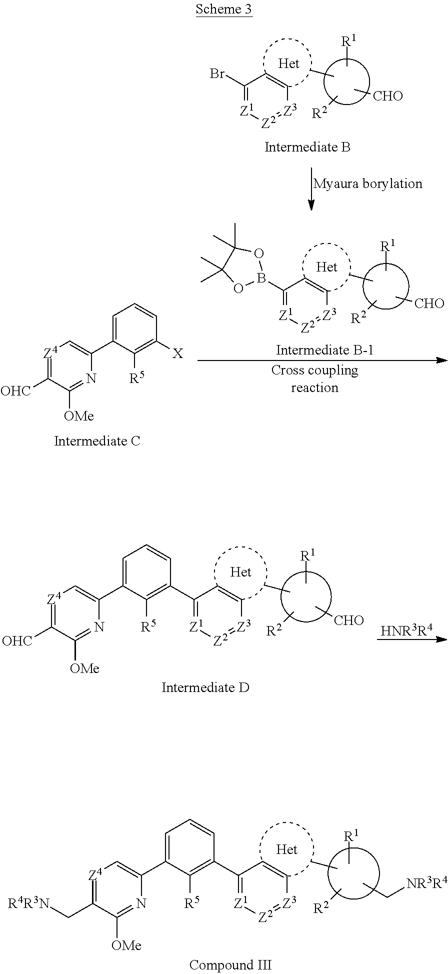

Compound III may be prepared according to Scheme 3. The preparation of dimeric Intermediate D may occur via a Suzuki cross coupling reaction in a mixture of 1,4-dioxane and H₂O of biaryl halide Intermediate C (X=Cl, Br, or I) and biaryl boronate Intermediate B-1 which may be synthesized via Miyaura borylation of Intermediate B in DMF. Reductive amination of both aldehyde groups of Intermediate D with amines of formula HNR³R⁴ in a mixture of MeOH and DCM may provide Compound III. The remaining protecting group may be removed with known deprotection methods. Compound III may be purified by column chromatography (e.g., using silica gel (230-400 mesh) and/or Sephadex LH-20).

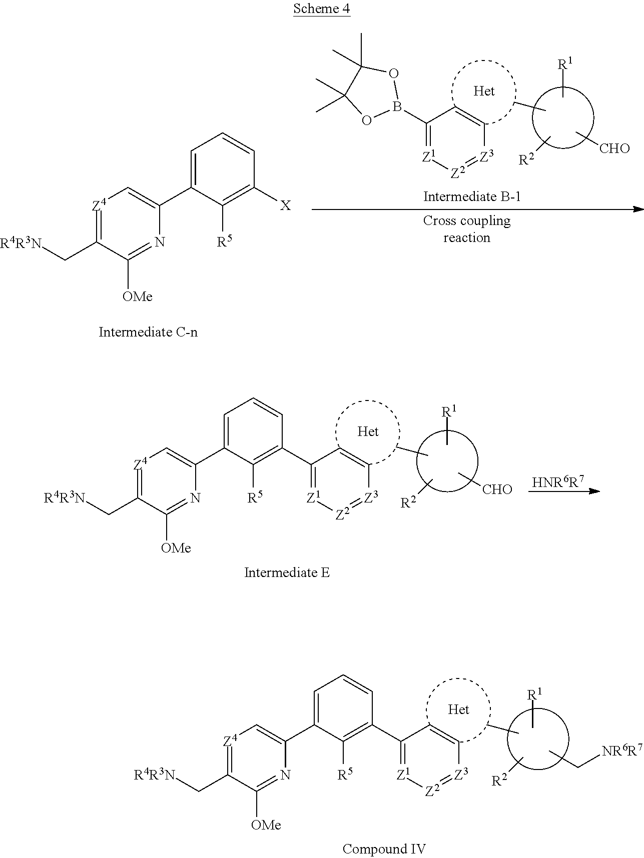

Compound IV may be prepared via the synthetic pathways depicted in Scheme 4 and Scheme 5. In Scheme 4 amine biaryl halide Intermediate C-n (X=Cl, Br, or I) reacts with biaryl boronate Intermediate B-1 under Suzuki cross coupling conditions with a mixture of 1,4-dioxane and H₂O to afford intermediate E. Intermediates E may be converted to Compound IV via a reductive amination with an amine of formula HWR⁶R⁷ in a mixture of MeOH and DCM. The remaining protecting group may be removed with known deprotection methods. Compound IV may be purified by column chromatography (e.g., using silica gel (230-400 mesh) and/or Sephadex LH-20).

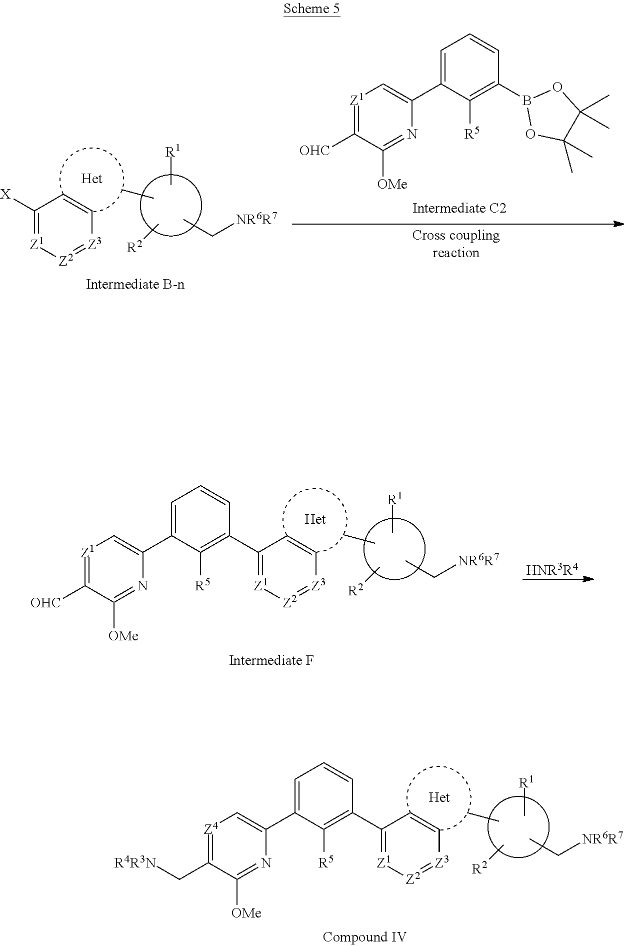

In Scheme 5, the Suzuki cross coupling reaction of amine biaryl halide Intermediate B-n (X=Cl, Br, or 1) and biaryl boronate Intermediate C2 in mixture of 1,4-dioxane and H₂O may afford Intermediate F. Intermediates F may be converted to Compound 1V via a reductive amination with an amine of formula HNR³R⁴ in a mixture of MeOH and DCM. The remaining protecting group may be removed with known deprotection methods. Compound IV may be purified by column chromatography (e.g., using silica gel (230-400 mesh) and/or Sephadex LH-20).

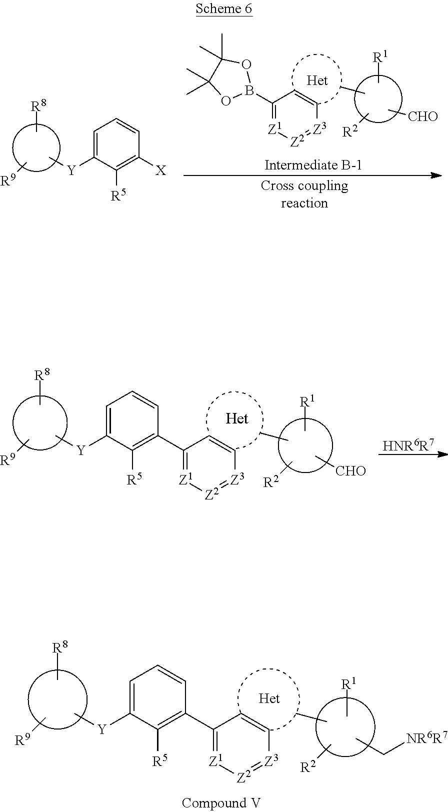

Compound V may be prepared according to Scheme 6. Suzuki cross coupling of aryl halides (X=Cl, Br, or I; Y=bond, amine, or amide) and biaryl boronate Intermediate B-1 in a mixture of 1,4-dioxane and H₂O may result in an asymmetric dimer intermediate, which can be converted to Compound V via the reductive amination with amines of formula HNR⁶R⁷ in a mixture of MeOH and DCM. The remaining protecting group may be removed with known deprotection methods. Compound V may be purified by column chromatography (e.g., using silica gel (230-400 mesh) and/or Sephadex LH-20).

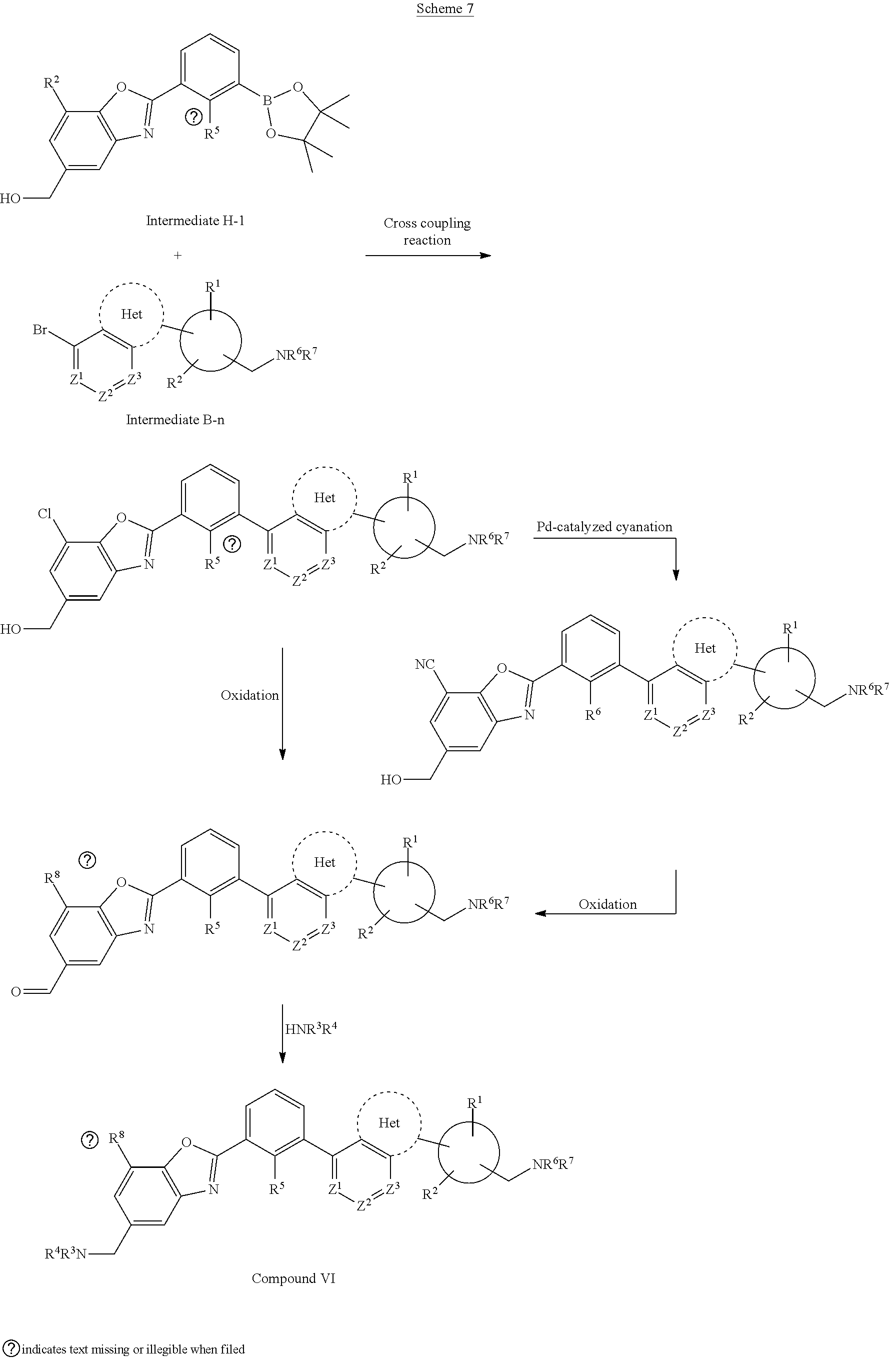

Compound VI may be prepared according to Scheme 7. The Suzuki cross coupling reaction of amine biaryl halide Intermediate B-n (X=Cl, Br, or I) and biaryl benzoxazole boronate Intermediate G-1 in mixture of 1,4-dioxane and H₂O may afford an asymmetric dimer intermediate. This intermediate may be converted to cyano intermediate via a Pd-catalyzed cyanation before both alcohol intermediates undergo oxidation to provide aldehyde intermediates (R⁸=Cl or CN). Aldehyde group of these intermediates may be converted to Compound VI via a reductive amination with an amine of formula HNR³R⁴ in a mixture of MeOH and DCM. The resulting compound may be purified by column chromatography (e.g., using silica gel (230-400 mesh) and/or Sephadex LH-20).

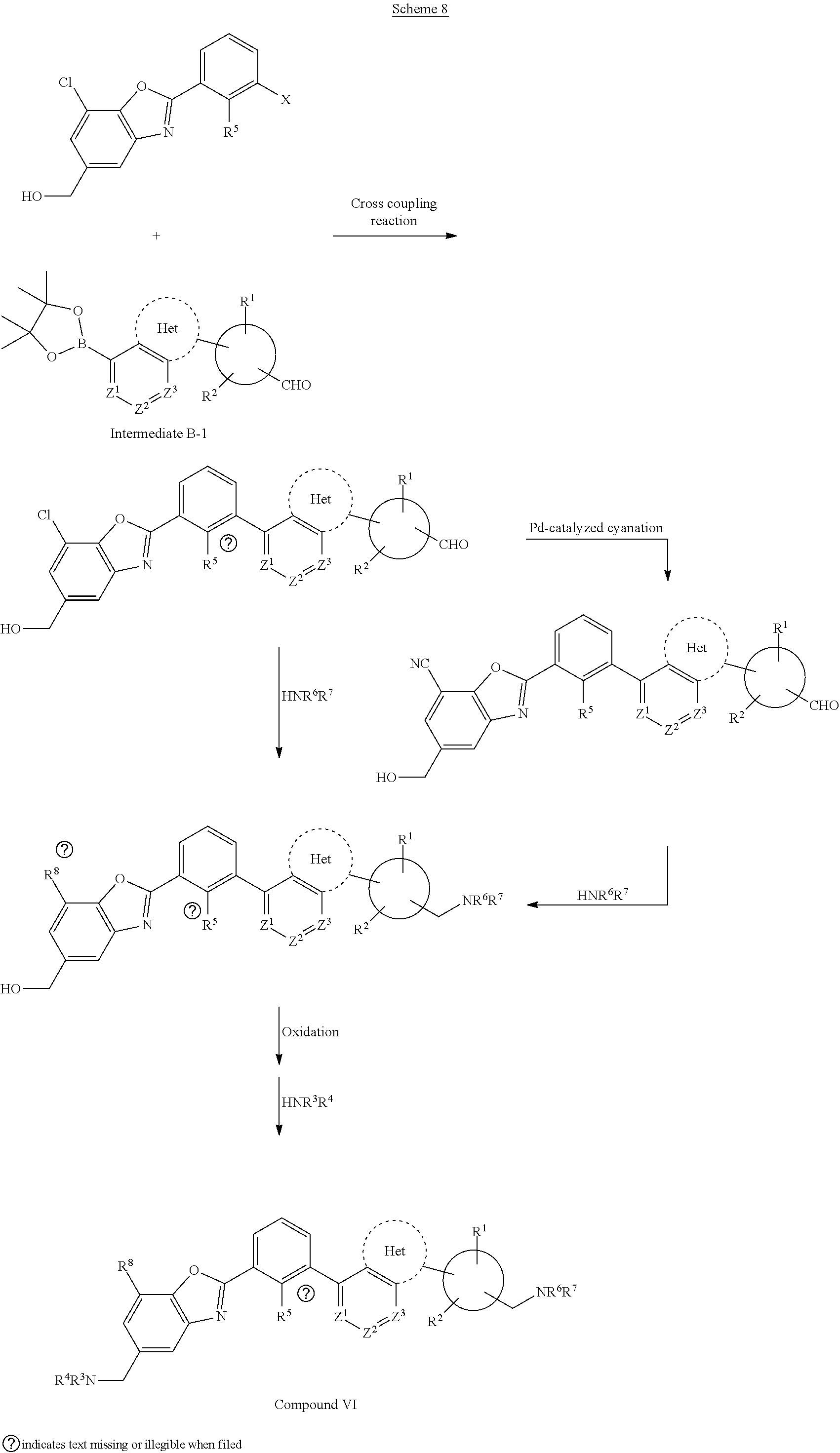

Compound VI may be prepared via another synthetic pathway as depicted in Scheme 8. In this synthetic route, biaryl boronate Intermediate B-1 reacts with biaryl benzoxazole halide (X=Br or I) via Suzuki cross coupling conditions with a mixture of 1,4-dioxane and H₂O to afford an asymmetric dimer intermediate that may be converted to cyano intermediate via a Pd-catalyzed cyanation. A reductive amination with an amine of formula HNR⁶R⁷ in a mixture of MeOH and DCM of both formyl-alcohol intermediates (R⁸=Cl or CN) may afford amine dimer alcohol intermediates. The oxidation of primary alcohol of these intermediates may result in aldehyde intermediates which can be converted to Compound VI via a reductive amination with an amine of formula HNR³R⁴ in a mixture of MeOH and DCM. The resulting compound may be purified by column chromatography (e.g., using silica gel (230-400 mesh) and/or Sephadex LH-20).

### Biological Assays

Compounds designed, selected and/or optimized by methods described above, once produced, can be characterized using a variety of assays known to those skilled in the art to determine whether the compounds have biological activity. For example, the molecules can be characterized by conventional assays, including but not limited to those assays described below, to determine whether they have a predicted activity, binding activity and/or binding specificity.

Furthermore, high-throughput screening can be used to speed up analysis using such assays. As a result, it can be possible to rapidly screen the molecules described herein for activity, using techniques known in the art. General methodologies for performing high-throughput screening are described, for example, in Devlin (1998) High Throughput Screening, Marcel Dekker; and U.S. Pat. No. 5,763,263. High-throughput assays can use one or more different assay techniques including, but not limited to, those described below.

Various in vitro or in vivo biological assays may be suitable for detecting the effect of the compounds of the present disclosure. These in vitro or in vivo biological assays can include, but are not limited to, enzymatic activity assays, electrophoretic mobility shift assays, reporter gene assays, in vitro cell viability assays, and the assays described herein.

In some embodiments, the compounds are analyzed with a protein-protein interaction assay. In some embodiments, the protein-protein interaction assay determines the interaction between PD-1 and PD-L1 of compounds of the present disclosure.

The compounds of the present disclosure can be analyzed using Amplified Luminescent Proximity Homogenous Assay to evaluate the blocking interaction between PD-1 and PD-L1 for the compounds of the present disclosure. The assay may be performed by incubating the compounds at various concentrations with tagged recombinant human PD-L1 protein and recombinant PD-1 protein. After the preincubation, chelate donor beads and Protein A acceptor beads may be added to the incubation mixture (e.g., under reduced light at room temperature for about 120 minutes). The signal may be measured with a plate reader and the IC₅₀ value calculated from the four-parameter logistic curve fit.

In some embodiments, the compounds are analyzed with a blockage cell-based bioassay. In some embodiments, the blocking interaction assay determines the blocking interaction between PD-1 and PD-L1 of compounds of the present disclosure.

The compounds of the present disclosure may be tested in a bioluminescent cell-based assay for PD-1/PD-L1 interaction blocking. Cells expressing PD-1 and luciferase gene reporter may be co-cultured with cells expressing human PD-L1 and surface-bound TCR activator.

Blocking of PD-1/PD-L1 interaction may prevent the inhibitory signal from PD-1 and increase NFAT-mediated luminescence. PD-L1 expressing cells may be seeded for a period of time (e.g., overnight). Compounds of the present disclosure may be added and incubated for a period of time at a temperature (e.g., for about 2 hours at about 37° C.). PD-1 expressing cells may be diluted in an assay medium added to each well. After co-incubation, luminescence may be determined by adding reagent followed by measurement with a luminescence plate reader. EC₅₀ values may be calculated from the four-parameter concentration-response curves.

In some embodiments, the biological assay is described in the Examples herein.

### Pharmaceutical Compositions

In some aspects, the present disclosure provides a pharmaceutical composition comprising a compound of the present disclosure as an active ingredient. In some embodiments, the present disclosure provides a pharmaceutical composition comprising at least one compound of each of the formulae described herein, or a pharmaceutically acceptable salt or solvate thereof, and one or more pharmaceutically acceptable carriers or excipients. In some embodiments, the present disclosure provides a pharmaceutical composition comprising at least one compound selected from Table 1.

As used herein, the term “composition” is intended to encompass a product comprising the specified ingredients in the specified amounts, as well as any product which results, directly or indirectly, from combination of the specified ingredients in the specified amounts.

The compounds of present disclosure can be formulated for oral administration in forms such as tablets, capsules (each of which includes sustained release or timed release formulations), pills, powders, granules, elixirs, tinctures, suspensions, syrups and emulsions. The compounds of present disclosure on can also be formulated for intravenous (bolus or in-fusion), intraperitoneal, topical, subcutaneous, intramuscular or transdermal (e.g., patch) administration, all using forms well known to those of ordinary skill in the pharmaceutical arts.

The formulation of the present disclosure may be in the form of an aqueous solution comprising an aqueous vehicle. The aqueous vehicle component may comprise water and at least one pharmaceutically acceptable excipient. Suitable acceptable excipients include those selected from the group consisting of a solubility enhancing agent, chelating agent, preservative, tonicity agent, viscosity/suspending agent, buffer, and pH modifying agent, and a mixture thereof.

Any suitable solubility enhancing agent can be used. Examples of a solubility enhancing agent include cyclodextrin, such as those selected from the group consisting of hydroxypropyl-3-cyclodextrin, methyl-β-cyclodextrin, randomly methylated-β-cyclodextrin, ethylated-β-cyclodextrin, triacetyl-β-cyclodextrin, peracetylated-β-cyclodextrin, carboxymethyl-β-cyclodextrin, hydroxyethyl-β-cyclodextrin, 2-hydroxy-3-(trimethylaminonio)propyl-3-cyclodextrin, glucosyl-β-cyclodextrin, sulfated β-cyclodextrin (S-β-CD), maltosyl-β-cyclodextrin, β-cyclodextrin sulfobutyl ether, branched-3-cyclodextrin, hydroxypropyl-γ-cyclodextrin, randomly methylated-γ-cyclodextrin, and trimethyl-γ-cyclodextrin, and mixtures thereof.

Any suitable chelating agent can be used. Examples of a suitable chelating agent include those selected from the group consisting of ethylenediaminetetraacetic acid and metal salts thereof, disodium edetate, trisodium edetate, and tetrasodium edetate, and mixtures thereof.

Any suitable preservative can be used. Examples of a preservative include those selected from the group consisting of quaternary ammonium salts such as benzalkonium halides (preferably benzalkonium chloride), chlorhexidine gluconate, benzethonium chloride, cetyl pyridinium chloride, benzyl bromide, phenylmercury nitrate, phenylmercury acetate, phenylmercury neodecanoate, merthiolate, methylparaben, propylparaben, sorbic acid, potassium sorbate, sodium benzoate, sodium propionate, ethyl p-hydroxybenzoate, propylaminopropyl biguanide, and butyl-p-hydroxybenzoate, and sorbic acid, and mixtures thereof.

The aqueous vehicle may also include a tonicity agent to adjust the tonicity (osmotic pressure). The tonicity agent can be selected from the group consisting of a glycol (such as propylene glycol, diethylene glycol, triethylene glycol), glycerol, dextrose, glycerin, mannitol, potassium chloride, and sodium chloride, and a mixture thereof.

The aqueous vehicle may also contain a viscosity/suspending agent. Suitable viscosity/suspending agents include those selected from the group consisting of cellulose derivatives, such as methyl cellulose, ethyl cellulose, hydroxyethylcellulose, polyethylene glycols (such as polyethylene glycol 300, polyethylene glycol 400), carboxymethyl cellulose, hydroxypropylmethyl cellulose, and cross-linked acrylic acid polymers (carbomers), such as polymers of acrylic acid cross-linked with polyalkenyl ethers or divinyl glycol (Carbopols—such as Carbopol 934, Carbopol 934P, Carbopol 971, Carbopol 974 and Carbopol 974P), and a mixture thereof.

In order to adjust the formulation to an acceptable pH (typically a pH range of about 5.0 to about 9.0, more preferably about 5.5 to about 8.5, particularly about 6.0 to about 8.5, about 7.0 to about 8.5, about 7.2 to about 7.7, about 7.1 to about 7.9, or about 7.5 to about 8.0), the formulation may contain a pH modifying agent. The pH modifying agent is typically a mineral acid or metal hydroxide base, selected from the group of potassium hydroxide, sodium hydroxide, and hydrochloric acid, and mixtures thereof, and preferably sodium hydroxide and/or hydrochloric acid. These acidic and/or basic pH modifying agents are added to adjust the formulation to the target acceptable pH range. Hence it may not be necessary to use both acid and base—depending on the formulation, the addition of one of the acid or base may be sufficient to bring the mixture to the desired pH range.

The aqueous vehicle may also contain a buffering agent to stabilize the pH. When used, the buffer is selected from the group consisting of a phosphate buffer (such as sodium dihydrogen phosphate and disodium hydrogen phosphate), a borate buffer (such as boric acid, or salts thereof including disodium tetraborate), a citrate buffer (such as citric acid, or salts thereof including sodium citrate), and ε-aminocaproic acid, and mixtures thereof.

The formulation may further comprise a wetting agent. Suitable classes of wetting agents include those selected from the group consisting of polyoxypropylene-polyoxyethylene block copolymers (poloxamers), polyethoxylated ethers of castor oils, polyoxyethylenated sorbitan esters (polysorbates), polymers of oxyethylated octyl phenol (Tyloxapol), polyoxyl 40 stearate, fatty acid glycol esters, fatty acid glyceryl esters, sucrose fatty esters, and polyoxyethylene fatty esters, and mixtures thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

According to a further aspect of the disclosure there is provided a pharmaceutical composition which comprises a compound of the disclosure as defined hereinbefore, or a pharmaceutically acceptable salt, hydrate or solvate thereof, in association with a pharmaceutically acceptable diluent or carrier.

The compositions of the disclosure may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

The compositions of the disclosure may be obtained by conventional procedures using conventional pharmaceutical excipients, well known in the art. Thus, compositions intended for oral use may contain, for example, one or more coloring, sweetening, flavoring and/or preservative agents.

An effective amount of a compound of the present disclosure for use in therapy is an amount sufficient to treat or prevent an inflammasome related condition referred to herein, slow its progression and/or reduce the symptoms associated with the condition.

An effective amount of a compound of the present disclosure for use in therapy is an amount sufficient to treat an inflammasome related condition referred to herein, slow its progression and/or reduce the symptoms associated with the condition.

The size of the dose for therapeutic or prophylactic purposes of a compound of Formula (I) will naturally vary according to the nature and severity of the conditions, the age and sex of the animal or patient and the route of administration, according to well-known principles of medicine.

### Methods of Use

In some aspects, the present disclosure provides a method of modulating PD-1 activity, PD-L1 activity, and/or the PD-1/PD-L1 interaction (e.g., in vitro or in vivo), comprising contacting a cell or protein with an effective amount of a compound of the present disclosure or a pharmaceutically acceptable salt thereof.

In some aspects, the present disclosure provides a method of modulating PD-1 activity (e.g., in vitro or in vivo), comprising contacting a cell or protein with an effective amount of a compound of the present disclosure or a pharmaceutically acceptable salt thereof.

In some aspects, the present disclosure provides a method of modulating PD-L1 activity (e.g., in vitro or in vivo), comprising contacting a cell or protein with an effective amount of a compound of the present disclosure or a pharmaceutically acceptable salt thereof.

In some aspects, the present disclosure provides a method of modulating PD-1 and PD-L1 interaction (e.g., in vitro or in vivo), comprising contacting a cell or protein with an effective amount of a compound of the present disclosure or a pharmaceutically acceptable salt thereof.

In some aspects, the present disclosure provides a method of treating or preventing a disease or disorder disclosed herein in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating a disease or disorder disclosed herein in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating or preventing a disease or disorder disclosed herein in a subject in need thereof, comprising administering to the subject a compound of the present disclosure or a pharmaceutically acceptable salt thereof, or a pharmaceutical composition of the present disclosure.

In some aspects, the present disclosure provides a method of treating a disease or disorder disclosed herein in a subject in need thereof, comprising administering to the subject a compound of the present disclosure or a pharmaceutically acceptable salt thereof, or a pharmaceutical composition of the present disclosure.

In some embodiments, the disease or disorder is associated with an implicated PD-1 activity, PD-L1 activity, and/or the PD-1/PD-L1 interaction. In some embodiments, the disease or disorder is a disease or disorder in which PD-1 activity, PD-L1 activity, and/or the PD-1/PD-L1 interaction is implicated.

In some embodiments, the disease or disorder is cancer.

In some aspects, the present disclosure provides a method of treating or preventing cancer in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating cancer in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating or preventing cancer in a subject in need thereof, comprising administering to the subject a compound of the present disclosure or a pharmaceutically acceptable salt thereof, or a pharmaceutical composition of the present disclosure.

In some aspects, the present disclosure provides a method of treating cancer in a subject in need thereof, comprising administering to the subject a compound of the present disclosure or a pharmaceutically acceptable salt thereof, or a pharmaceutical composition of the present disclosure.

In some aspects, the present disclosure provides a compound of the present disclosure or a pharmaceutically acceptable salt thereof for use in modulating PD-1 activity, PD-L1 activity, and/or PD-1/PD-L1 interaction (e.g., in vitro or in vivo).

In some aspects, the present disclosure provides a compound of the present disclosure or a pharmaceutically acceptable salt thereof for use in modulating PD-1 activity (e.g., in vitro or in vivo).

In some aspects, the present disclosure provides a compound of the present disclosure or a pharmaceutically acceptable salt thereof for use in modulating PD-L1 activity (e.g., in vitro or in vivo).

In some aspects, the present disclosure provides a compound of the present disclosure or a pharmaceutically acceptable salt thereof for use in modulating PD-1 and PD-L1 interaction (e.g., in vitro or in vivo).

In some aspects, the present disclosure provides a compound of the present disclosure or a pharmaceutically acceptable salt thereof for use in treating or preventing a disease or disorder disclosed herein.

In some aspects, the present disclosure provides a compound of the present disclosure or a pharmaceutically acceptable salt thereof for use in treating a disease or disorder disclosed herein.

In some aspects, the present disclosure provides a compound of the present disclosure or a pharmaceutically acceptable salt thereof for use in treating or preventing cancer in a subject in need thereof.

In some aspects, the present disclosure provides a compound of the present disclosure or a pharmaceutically acceptable salt thereof for use in treating cancer in a subject in need thereof.

In some aspects, the present disclosure provides use of a compound of the present disclosure or a pharmaceutically acceptable salt thereof in the manufacture of a medicament for modulating PD-1 activity, PD-L1 activity, and/or the PD-1/PD-L1 interaction (e.g., in vitro or in vivo).

In some aspects, the present disclosure provides use of a compound of the present disclosure or a pharmaceutically acceptable salt thereof in the manufacture of a medicament for modulating PD-1 activity (e.g., in vitro or in vivo).

In some aspects, the present disclosure provides use of a compound of the present disclosure or a pharmaceutically acceptable salt thereof in the manufacture of a medicament for modulating PD-L1 activity (e.g., in vitro or in vivo).

In some aspects, the present disclosure provides use of a compound of the present disclosure or a pharmaceutically acceptable salt thereof in the manufacture of a medicament for modulating PD-1 and PD-L1 interaction (e.g., in vitro or in vivo).

In some aspects, the present disclosure provides use of a compound of the present disclosure or a pharmaceutically acceptable salt thereof in the manufacture of a medicament for treating or preventing a disease or disorder disclosed herein.

In some aspects, the present disclosure provides use of a compound of the present disclosure or a pharmaceutically acceptable salt thereof in the manufacture of a medicament for treating a disease or disorder disclosed herein.

In some aspects, the present disclosure provides use of a compound of the present disclosure or a pharmaceutically acceptable salt thereof in the manufacture of a medicament for treating or preventing cancer in a subject in need thereof.

In some aspects, the present disclosure provides use of a compound of the present disclosure or a pharmaceutically acceptable salt thereof in the manufacture of a medicament for treating cancer in a subject in need thereof.

The present disclosure provides compounds that function as modulators of PD-1 activity, PD-L1 activity, and/or the PD-1/PD-L1 interaction.

In some embodiments, modulation is inhibition.

Effectiveness of compounds of the disclosure can be determined by industry-accepted assays/disease models according to standard practices of elucidating the same as described in the art and are found in the current general knowledge.

The present disclosure also provides a method of treating a disease or disorder in which PD-1 activity, PD-L1 activity, and/or the PD-1/PD-L1 interaction is implicated in a patient in need of such treatment, said method comprising administering to said patient a therapeutically effective amount of a compound, or a pharmaceutically acceptable salt thereof, or a pharmaceutical composition as defined herein.

The present disclosure also provides a method of treating a disease or disorder in which PD-1 activity, PD-L1 activity, and/or the PD-1/PD-L1 interaction is implicated in a patient in need of such treatment, said method comprising administering to said patient a compound, or a pharmaceutically acceptable salt thereof, or a pharmaceutical composition as defined herein.

In some embodiments, the disease or disorder is cancer.

In some embodiments, the cancer is selected from brain and spinal cancers, cancers of the head and neck, leukemia and cancers of the blood, skin cancers, cancers of the reproductive system, cancers of the gastrointestinal system, liver and bile duct cancers, kidney and bladder cancers, bone cancers, lung cancers, malignant mesothelioma, sarcomas, lymphomas, glandular cancers, thyroid cancers, heart tumors, germ cell tumors, malignant neuroendocrine (carcinoid) tumors, midline tract cancers, and cancers of unknown primary (i.e., cancers in which a metastasized cancer is found but the original cancer site is not known). In some embodiments, the cancer is anaplastic astrocytomas, glioblastomas, astrocytomas, or estheosioneuroblastomas (also known as olfactory blastomas). In some embodiments, the cancer is head or neck cancer, including, but not limited to, nasopharyngeal cancers, nasal cavity and paranasal sinus cancers, hypopharyngeal cancers, oral cavity cancers (e.g., squamous cell carcinomas, lymphomas, and sarcomas), lip cancers, oropharyngeal cancers, salivary gland tumors, cancers of the larynx (e.g., laryngeal squamous cell carcinomas, rhabdomyosarcomas), or cancers of the eye or ocular cancers.

In some embodiments, the cancer is leukemia or cancers of the blood. In some embodiments, the cancer is myeloproliferative neoplasms, myelodysplastic syndromes, myelodysplastic/myeloproliferative neoplasms, acute myeloid leukemia (AML), myelodysplastic syndrome (MDS), chronic myelogenous leukemia (CML), myeloproliferative neoplasm (MPN), post-MPN AML, post-MDS AML, del(5q)-associated high risk MDS or AML, blast-phase chronic myelogenous leukemia, angioimmunoblastic lymphoma, acute lymphoblastic leukemia, Langerans cell histiocytosis, hairy cell leukemia, or plasma cell neoplasms, including, but not limited to plasmacytomas or multiple myelomas. In some embodiments, the leukemia is acute or chronic. In some embodiments, the skin cancer is melanoma, squamous cell cancers, or basal cell cancers.

In some embodiments, the cancer is cancers of the reproductive system. In some embodiments, the cancer is breast cancers, cervical cancers, vaginal cancers, ovarian cancers, prostate cancers, penile cancers, or testicular cancers.

In some embodiments, the cancer is cancers of the gastro-intestinal system. In some embodiments, the cancer is esophageal cancers, gastric cancers (also known as stomach cancers), gastrointestinal carcinoid tumors, pancreatic cancers, gallbladder cancers, colorectal cancers, or anal cancer. In some embodiments, the cancer is esophageal squamous cell carcinomas, esophageal adenocarcinomas, gastric adenocarcinomas, gastrointestinal carcinoid tumors, gastrointestinal stromal tumors, gastric lymphomas, gastrointestinal lymphomas, solid pseudopapillary tumors of the pancreas, pancreatoblastoma, islet cell tumors, pancreatic carcinomas including acinar cell carcinomas and ductal adenocarcinomas, gallbladder adenocarcinomas, colorectal adenocarcinomas, or anal squamous cell carcinomas. In some embodiments, the cancer is liver or bile duct cancer. In some embodiments, the cancer is kidney or bladder cancers.

In some embodiments, the cancer is bone cancer. In some embodiments, the bone cancer is osteosarcoma, malignant fibrous histiocytoma of bone, Ewing sarcoma, or chordoma (cancer of the bone along the spine).

In some embodiments, the cancer is lung cancer. In some embodiments, the lung cancer is non-small cell lung cancer (NSCLC), small cell lung cancers, bronchial tumors, or pleuropulmonary blastomas. In some embodiments, the cancer is malignant mesothelioma.

In some embodiments, the cancer is sarcoma. In some embodiments, the sarcoma is central chondrosarcoma, central and periosteal chondroma, fibrosarcoma, clear cell sarcoma of tendon sheaths, or Kaposi's sarcoma. In some embodiments, the cancer is lymphoma. In some embodiments, the cancer is Hodgkin lymphoma (e.g., Reed-Sternberg cells), non-Hodgkin lymphoma (e.g., diffuse large B-cell lymphoma, follicular lymphoma, mycosis fungoides, Sezary syndrome, primary central nervous system lymphoma), cutaneous T-cell lymphomas, or primary central nervous system lymphomas.

In some embodiments, the present disclosure provides use of a compound of the present disclosure, or a pharmaceutically acceptable salt thereof, in combination with at least one additional anti-cancer agent or therapy.

In some embodiments, the present disclosure provides use of a compound of the present disclosure, or a pharmaceutically acceptable salt thereof, in combination with one additional anti-cancer agent or therapy.

In some embodiments, the present disclosure provides use of a compound of the present disclosure, or a pharmaceutically acceptable salt thereof, in combination with two additional anti-cancer agent or therapy.

In some embodiments, the anti-cancer agent or therapy is rituxan, chemotherapy (e.g., doxorubicin, gemcitabine), a check-point inhibitor (e.g., nivolumab, pembrolizumab, atezolizumab, and ipilimumab), radiation therapy, or resection therapy.

In some embodiments, the present disclosure provides a pharmaceutical composition comprising a compound of the present disclosure or a pharmaceutically acceptable salt thereof, at least one additional anti-cancer agent or therapy, and at least one pharmaceutically acceptable carrier or excipient.

### Routes of Administration

Compounds of the present disclosure, or pharmaceutically acceptable salts thereof, may be administered alone as a sole therapy or can be administered in addition with one or more other substances and/or treatments. Such conjoint treatment may be achieved by way of the simultaneous, sequential or separate administration of the individual components of the treatment.

For example, therapeutic effectiveness may be enhanced by administration of an adjuvant (i.e. by itself the adjuvant may only have minimal therapeutic benefit, but in combination with another therapeutic agent, the overall therapeutic benefit to the individual is enhanced).

Alternatively, by way of example only, the benefit experienced by an individual may be increased by administering the compound of Formula (I) with another therapeutic agent (which also includes a therapeutic regimen) that also has therapeutic benefit.

In the instances where the compound of the present disclosure is administered in combination with other therapeutic agents, the compound of the disclosure need not be administered via the same route as other therapeutic agents, and may, because of different physical and chemical characteristics, be administered by a different route. For example, the compound of the disclosure may be administered orally to generate and maintain good blood levels thereof, while the other therapeutic agent may be administered intravenously. The initial administration may be made according to established protocols known in the art, and then, based upon the observed effects, the dosage, modes of administration and times of administration can be modified by the skilled clinician.

The particular choice of other therapeutic agent will depend upon the diagnosis of the attending physicians and their judgment of the condition of the individual and the appropriate treatment protocol. According to this aspect of the disclosure there is provided a combination for use in the treatment of a disease in which inflammasome activity is implicated comprising a compound of the disclosure as defined hereinbefore, or a pharmaceutically acceptable salt thereof, and another suitable agent.

According to a further aspect of the disclosure there is provided a pharmaceutical composition which comprises a compound of the disclosure, or a pharmaceutically acceptable salt thereof, in combination with a suitable, in association with a pharmaceutically acceptable diluent or carrier.

In addition to its use in therapeutic medicine, compounds of Formula (I) and pharmaceutically acceptable salts thereof are also useful as pharmacological tools in the development and standardization of in vitro and in vivo test systems for the evaluation of the effects of modulators of PD-1 activity, PD-L1 activity, and/or the PD-1/PD-L1 interaction in laboratory animals such as dogs, rabbits, monkeys, rats and mice, as part of the search for new therapeutic agents.

In any of the above-mentioned pharmaceutical composition, process, method, use, medicament, and manufacturing features of the instant disclosure, any of the alternate embodiments of macromolecules of the present disclosure described herein also apply.

The compounds of the disclosure or pharmaceutical compositions comprising these compounds may be administered to a subject by any convenient route of administration, whether systemically/peripherally or topically (i.e., at the site of desired action).

Routes of administration include, but are not limited to, oral (e.g. by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eye drops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intra-arterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

### EXAMPLES

For exemplary purpose, neutral compounds of Formula (I) are synthesized and tested in the examples. It is understood that the neutral compounds of Formula (I) may be converted to the corresponding pharmaceutically acceptable salts of the compounds using routine techniques in the art (e.g., by saponification of an ester to the carboxylic acid salt, or by hydrolyzing an amide to form a corresponding carboxylic acid and then converting the carboxylic acid to a carboxylic acid salt).

Nuclear magnetic resonance (NMR) spectra were recorded at 400 MHz or 300 MHz as stated and at 300.3 K unless otherwise stated; the chemical shifts (S) are reported in parts per million (ppm).

The High Performance Liquid Chromatography (HPLC) measurement was performed using a LC pump, a diode-array (DAD) or a UV detector and a column as specified in the respective methods. If necessary additional detectors were included (see table of methods below).

Flow from the column was brought to the Mass Spectrometer (MS) which was configured with an atmospheric pressure ion source. The tune parameters (e.g. scanning range, dwell time, collision energy . . . ) was set within the knowledge of the skilled person in order to obtain ions allowing the identification of the compounds monoisotopic molecular weight (MW). Data acquisition was performed with appropriate software.

The compound characterization is presented by experimental retention times (RT) and ions. The reported molecular ion corresponds to the [M+H]⁺ (protonated molecule) and/or [M−H]⁻ (deprotonated molecule). In case the compound was not directly ionizable the type of adduct is specified (i.e. [M+NH₄⁺]⁺, [M+Na]⁺, [M+HCOO]⁻, etc.). All results were obtained with experimental uncertainties that are commonly associated with the method used.

Hereinafter, “SQD” means Single Quadrupole Detector, “Q-Tof” Quadrupole Time-of-flight mass spectrometers, “DAD” Diode Array Detector, “rt” room temperature,

### LCMS Methods

Mobile Phase A=0.1% HCOOH+H₂O, Mobile Phase B=0.1% HCOOH+CH₃CN

| | | | Flow | Run | |
|---|---|---|---|---|---|
| | | | (mL/min) | time | |
| Method | Column | Gradient | Col T (° C.) | (min) | Instrument |
| A | Eclipse XDB- | From 90% A to 30% A in 1.0 min, | 0.75 | 8 | Agilent: |
| | Phenyl, 3.5 μm, | From 30% A to 15% A in 3.0 min, | 45 | | 1260 |
| | 100 × 3.0 mm | held for 1.0 min, From 15% A to | | | Infinity II, - |
| | (Agilent) | 90% A in 1.0 min, held for 2.0 min. | | | DAD, SQD |
| | | | | | (LC/MSD) |
| B | Eclipse XDB- | 90% A held for 0.5 min, From 90% | 0.6 | 9 | Agilent: |
| | Phenyl, 3.5 μm, | A to 30% A in 0.5 min, From 30% | 45 | | 1260 |
| | 100 × 3.0 mm | A to 15% A in 3.0 min, held for | | | Infinity II, - |
| | (Agilent) | 1.0 min, From 15% A to 30% A in | | | DAD, SQD |
| | | 1.5 min, From 30% A to 90% A in | | | (LC/MSD) |
| | | 1.5 min, held for 1.0 min. | | | |
| C | Eclipse XDB- | 90% A held for 1.0 min, From 90% | 0.6 | 12 | Agilent: |
| | Phenyl, 3.5 μm, | A to 30% A in 1.0 min, From 30% | 45 | | 1260 |
| | 100 × 3.0 mm | A to 15% A in 4.0 min, held for | | | Infinity II, - |
| | (Agilent) | 1.0 min, From 15% A to 30% A in | | | DAD, SQD |
| | | 2.0 min, From 30% A to 90% A in | | | (LC/MSD) |
| | | 1.0 min, held for 2.0 min. | | | |
| D | Kinetic C-18, | 98% A held for 0.5 min, From 98% | 0.3 | 7 | Sciex: |
| | 1.3 or 1.7 μm, | A to 35% A in 4.0 min, From 35% | 45 | | ExionLC ™ |
| | 50 × 2.1 mm | A to 5% A in 0.5 min, held for | | | and Q-Tof |
| | (Phenonenex) | 1.0 min, From 5% A to 98% A in | | | (X500B) |
| | | 0.1 min, held for 0.9 min. | | | |
| E | Kinetic C-18, | 80% A held for 0.5 min. From 80% | 0.2 | 10 | Sciex: |
| | 1.3 μm, | A to 10% A in 6.0 min, held for | 45 | | ExionLC ™ |
| | 50 × 2.1 mm | 1.0 min. From 10% A to 80% A in | | | and SQD |
| | (Phenonenex) | 0.5 min, held for 2.0 min, | | | (QTRAP |
| | | | | | 6500+) |
| F | Eclipse XDB- | 90% A held for 1.0 min, From 90% | 0.6 | 17 | Agilent: |
| | Phenyl, 3.5 μm, | A to 70% A in 1.0 min, held for | 45 | | 1260 |
| | 100 × 3.0 mm | 2.0 min. From 70% A to 50% A in | | | Infinity II, - |
| | (Agilent) | 1.0 min, held for 4.0 min, From | | | DAD, SQD |
| | | 50% A to 30% A in 1.0 min, held | | | (LC/MSD) |
| | | for 1.0 min. From 30% A to 15% A | | | |
| | | in 1.0 min, held for 1.0 min. | | | |
| | | From 15% A to 30% A in 1.0 min. | | | |
| | | From 30% A to 90% A in 1.0 min, | | | |
| | | held for 2.0 min. | | | |
| G | Eclipse XDB- | 90% A held for 1.0 min, From 90% | 0.6 | 17 | Agilent: |
| | Phenyl, 3.5 μm, | A to 70% A in 2.0 min, held for | 45 | | 1260 |
| | 100 × 3.0 mm | 3.0 min. From 70% A to 50% A in | | | Infinity II, - |
| | (Agilent) | 4.0 min, beld for 1.0 min, From | | | DAD, SQD |
| | | 50% A to 15% A in 1.0 min, held | | | (LC/MSD) |
| | | for 1.0 min. From 15% A to 30% A | | | |
| | | in 1.0 min. From 30% A to 90% A | | | |
| | | in 1.0 min, held for 2.0 min. | | | |

*Col T = Column temperature

### Abbreviations

ACN acetonitrile
AcOH acetic acid
B₂Pin₂ Bis(pinacolato)diboron
DCE 1,2-dichloroethane
DCM dichloromethane
DIAD diisopropyl azodicarboxylate
DIPEA N,N-diisopropylethylamine
DMAP N,N-dimethylaminopyridine
DMF N,N-dimethylformamide
DPPA diphenylphosphoryl azide
dppf 1,1′-bis(diphenylphosphino)ferrocene
EDCI N-(3-dimethylaminopropyl)-N′-ethylcarbodiimide hydrochloride
ESI electrospray ionisation
EtOAc ethyl acetate
EtOH ethanol
h hour(s)
HATU N-[(dimethylamino)-1H-1,2,3-triazolo-[4,5-b]pyridin-1-ylmethylene]-N-methylmethanaminium hexafluorophosphate N-oxide
HMPA hexamethylphosphoramide
HOBt hydroxybenzotriazole
Jones reagent CrO₃ in aqueous H₂SO₄
K₂CO₃ potassium carbonate
KOAc potassium acetate
Lawesson's reagent 2,4-Bis(4-methoxyphenyl)-1,3,2,4-dithiadiphosphetane-2,4 dithione
LCMS Liquid Chromatography-Mass Spectrometry
MeCN acetonitrile
MeOD methanol-d₄
MeOH methanol
Me₂S dimethyl sulfide
min minute(s)
m/z mass/charge
NBS N-bromosuccinimide
NCS N-chlorosuccinimide
Pd/C palladium on carbon
Pd₂(dba)₃ tris(dibenzylideneacetone)dipalladium(0)
Pd(PPh₃)₄ tetrakis(triphenylphosphine)palladium(0)
PPh₃ triphenylphosphine
prep-HPLC preparative high-performance liquid chromatography
prep-TLC preparative thin-layer chromatography
psi pound-force per square inch
Pt/C platinum on carbon
RM reaction mixture
rt room temperature
RT retention time
TFA trifluoroacetic acid
THF tetrahydrofuran
XPhos 2-dicyclohexylphosphino-2′,4′,6′-triisopropylbiphenyl
Y yield

### Synthesis of Intermediates

### Intermediate A1: 4-(2,3-dihydrobenzo[b] [1,4]dioxin-6-yl)-1H-indole

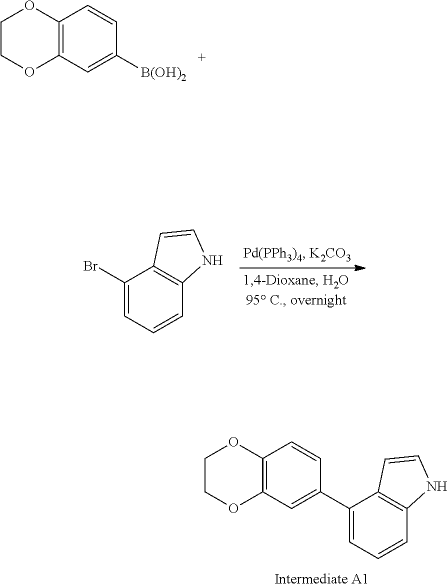 [SMILES: OB(O)c1ccc2c(c1)OCCO2 ; Brc1cccc2[nH]ccc12 ; c1cc(-c2ccc3c(c2)OCCO3)c2cc[nH]c2c1]

Pd(PPh₃)₄ (0.60 g, 0.52 mmol) was added to a solution of 4-bromoindole (1.01 g, 5.16 mmol) and 1,4-benzodioxane-6-boronic acid (1.12 g, 6.24 mmol) in 1,4-dioxane 16 mL. A solution of K₂CO₃ (1.72 g, 12.49 mmol) in H₂O 4 mL was added to the reaction mixture before it was sparged with argon for 10 min. After the reaction mixture was stirred at 95° C. overnight under argon atmosphere, it was cooled down and diluted with brine. The resulting mixture was extracted 3 times with EtOAc and the organic layer was collected, dried over anhydrous Na₂SO₄, filtered, and evaporated to dryness. The crude mixture was purified by column chromatography (silica gel, gradient elution, 0 to 20% EtOAc/hexanes) to provide Intermediate A1.

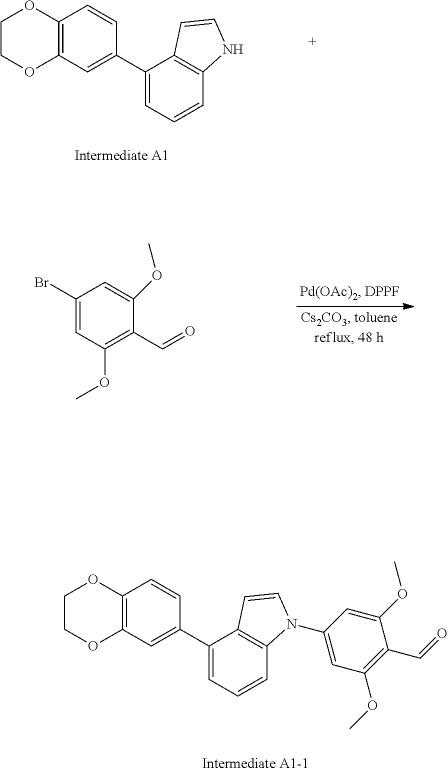 [SMILES: c1cc(-c2ccc3c(c2)OCCO3)c2cc[nH]c2c1 ; COc1cc(Br)cc(OC)c1C=O ; COc1cc(-n2ccc3c(-c4ccc5c(c4)OCCO5)cccc32)cc(OC)c1C=O]

### Intermediate A-1: 4-(4-(2,3-dihydrobenzo[b] [1,4]dioxin-6-yl)-1H-indol-1-yl)-2,6-dimethoxybenzaldehyde

A mixture of Intermediate A1 (0.77 g, 3.06 mmol), 4-bromo-2,6-dimethoxybenzaldehyde (1.12 g, 4.59 mmol), Pd(OAc)₂ (0.0347 g, 0.15 mmol), DPPF (0.11 g, 0.19 mmol) and Cs₂CO₃ (1.50 g, 4.60 mmol) in toluene 15 mL was sparged with argon for 10 min before it was refluxed under argon atmosphere for 48 h. The resulting mixture was cooled down, filtered, and evaporated to dryness. The crude mixture was purified by column chromatography (silica gel, gradient elution, 20 to 60% EtOAc/hexanes) to provide Intermediate A1-1.

### Intermediate A1-2: 3-((5-(4-(2,3-dihydrobenzo[b][1,4]dioxin-6-yl)-1H-indol-1-yl)-2-formylphenoxy)methyl)benzonitrile

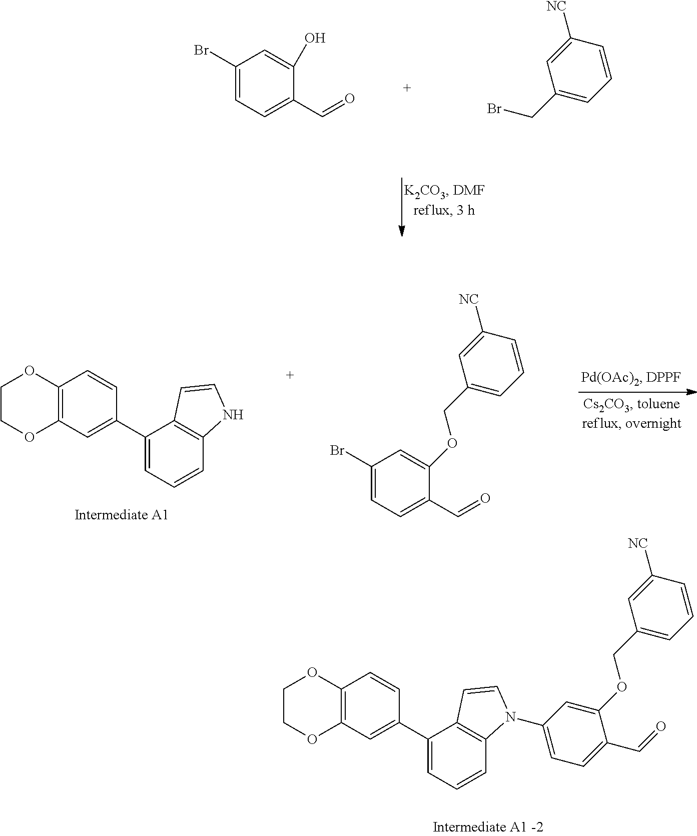 [SMILES: O=Cc1ccc(Br)cc1O ; N#Cc1cccc(CBr)c1 ; c1cc(-c2ccc3c(c2)OCCO3)c2cc[nH]c2c1 ; N#Cc1cccc(COc2cc(Br)ccc2C=O)c1 ; N#Cc1cccc(COc2cc(-n3ccc4c(-c5ccc6c(c5)OCCO6)cccc43)ccc2C=O)c1]

3-(Bromomethyl)benzonitrile (1.30 g, 6.63 mmol) was added to a mixture of 4-bromo-2-hydroxybenzaldehyde (1.20 g, 5.97 mmol) and K₂CO₃ (1.33 g, 9.62 mmol) in DMF 6 mL and the reaction mixture was refluxed for 3 h. Then, the mixture was cooled down and diluted with water. The resulting mixture was extracted 3 times with EtOAc and the organic layer was washed with brine, dried over anhydrous Na₂SO₄, filtered, and evaporated to dryness. The crude mixture was precipitated by DCM/hexanes to provide 3-((5-bromo-2-formylphenoxy)methyl)benzonitrile.

A mixture of Intermediate A1 (0.18 g, 0.72 mmol), 3-((5-bromo-2-formylphenoxy)methyl)benzonitrile (0.34 g, 1.08 mmol), Pd(OAc)₂ (9.00 mg, 0.04 mmol), DPPF (25.00 g, 0.05 mmol) and Cs₂CO₃ (361.00 g, 1.11 mmol) in toluene 7 mL was sparged with argon for 10 min before it was refluxed under argon atmosphere for overnight. The resulting mixture was cooled down, filtered, and evaporated to dryness. The crude mixture was purified by column chromatography (silica gel, gradient elution, 20 to 60% EtOAc/hexanes) to provide Intermediate A1-2.

### Intermediate A1-3: 6-(4-(2,3-dihydrobenzo[b][1,4]dioxin-6-yl)-1H-indol-1-yl)nicotinaldelhyde

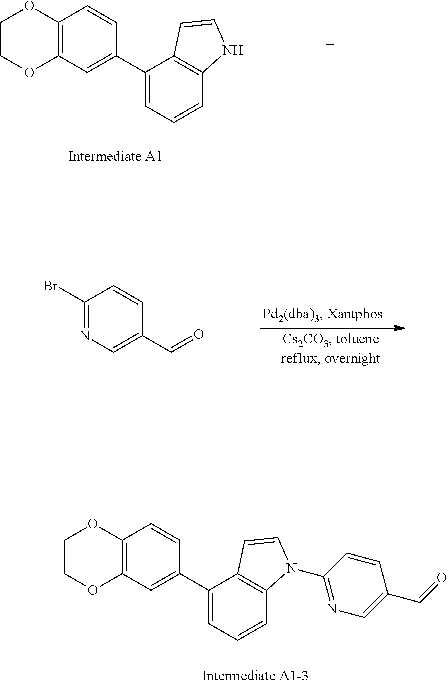 [SMILES: c1cc(-c2ccc3c(c2)OCCO3)c2cc[nH]c2c1 ; O=Cc1ccc(Br)nc1 ; O=Cc1ccc(-n2ccc3c(-c4ccc5c(c4)OCCO5)cccc32)nc1]

A mixture of Intermediate A1 (137.00 g, 0.55 mmol), 6-bromonicotinaldehyde (0.17 g, 0.92 mmol), Pd₂(dba)₃ (15.00 mg, 0.02 mmol), Xantphos (38.00 mg, 0.07 mmol) and Cs₂CO₃ (0.38 g, 1.16 mmol) in toluene 12 mL was sparged with argon for 10 min before it was refluxed under argon atmosphere for overnight. Then, the mixture was cooled down and diluted with water. The resulting mixture was extracted 3 times with EtOAc and the organic layer was collected, dried over anhydrous Na₂SO₄, filtered, and evaporated to dryness. The crude mixture was purified by column chromatography (silica gel, gradient elution, 0 to 60% EtOAc/hexanes) to provide Intermediate A1-3.

### Intermediate A1-4: 6-(4-(2,3-dilhydrobenzo[b] [1,4]dioxin-6-yl)-1H-indol-1-yl)-2-methoxynicodnaldelhyde

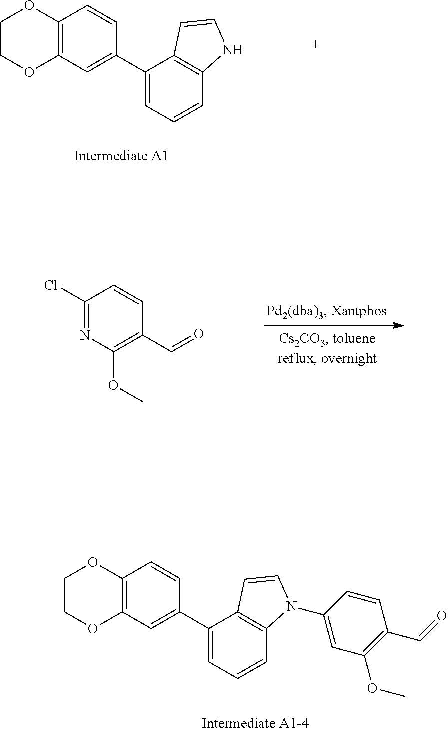 [SMILES: c1cc(-c2ccc3c(c2)OCCO3)c2cc[nH]c2c1 ; COc1nc(Cl)ccc1C=O ; COc1cc(-n2ccc3c(-c4ccc5c(c4)OCCO5)cccc32)ccc1C=O]

A mixture of Intermediate A1 (169.20 mg, 0.67 mmol), 6-chloro-2-methoxynicotinaldehyde (0.12 g, 0.68 mmol), Pd₂(dba)₃ (19.80 mg, 0.02 mmol), Xantphos (40.40 g, 0.07 mmol) and Cs₂CO₃ (43.92 mg, 1.35 mmol) in toluene 15 mL was sparged with argon for 10 min before it was refluxed under argon atmosphere for overnight. Then, the mixture was cooled down and diluted with water. The resulting mixture was extracted 3 times with EtOAc and the organic layer was collected, dried over anhydrous Na₂SO₄, filtered, and evaporated to dryness. The crude mixture was purified by column chromatography (silica gel, gradient elution, 0 to 60% EtOAc/hexanes) to provide Intermediate A1-4.

### Intermediate A1-5: 2-(4-(2,3-dihydrobenzo[b] [1,4]dioxin-6-yl)-1H-indol-1-yl)-6-methylisonicotinaldehyde

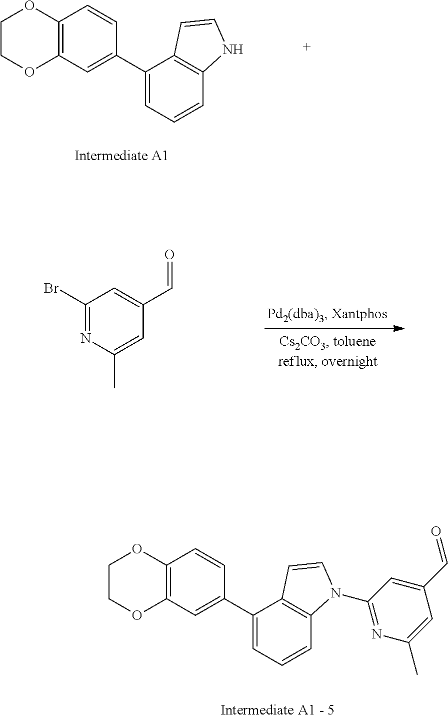

A mixture of Intermediate A1 (0.15 g, 0.59 mmol), 2-bromo-6-methylisonicotinaldehyde (0.12 g, 0.65 mmol), Pd₂(dba)₃ (0.03 g, 0.03 mmol), Xantphos (0.03 g, 0.06 mmol) and Cs₂CO₃ (0.68 g, 2.09 mmol) in toluene 10 mL was sparged with argon for 10 min before it was refluxed under argon atmosphere for overnight. Then, the mixture was cooled down and diluted with water. The resulting mixture was extracted 3 times with EtOAc and the organic layer was collected, dried over anhydrous Na₂SO₄, filtered, and evaporated to dryness. The crude mixture was purified by column chromatography (silica gel, gradient elution, 0 to 60% EtOAc/hexanes) to provide Intermediate A1-5.

### Intermediate A2: 4-(2,3-dihydrobenzo[b] [1,4]dioxin-6-yl)indoline

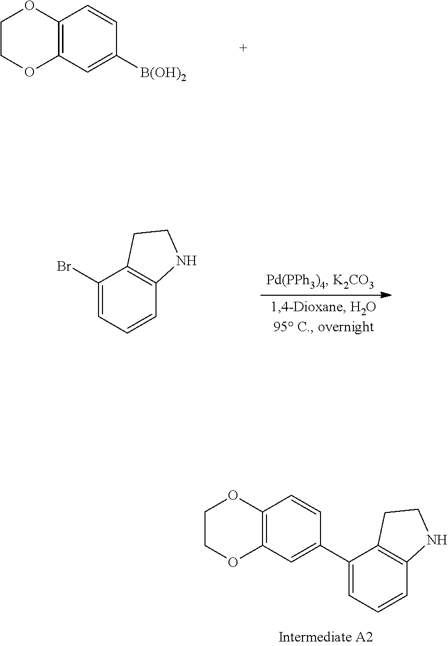 [SMILES: OB(O)c1ccc2c(c1)OCCO2 ; Brc1cccc2c1CCN2 ; c1cc2c(c(-c3ccc4c(c3)OCCO4)c1)CCN2]

Pd(PPh₃)₄ (0.59 g, 0.51 mmol) and K₂CO₃ (2.16 g, 15.63 mmol) were added to a solution of 4-bromoindoline (1.00 g, 5.05 mmol) and 1,4-benzodioxane-6-boronic acid (1.37 g, 7.61 mmol) in a mixture of 1,4-dioxane and H₂O (4:1) 20 mL. After the reaction mixture was sparged with argon for 10 min, it was stirred at 95° C. overnight under argon atmosphere. Then, the mixture was cooled down and diluted with brine. The resulting mixture was extracted 3 times with EtOAc and the organic layer was collected, dried over anhydrous Na₂SO₄, filtered, and evaporated to dryness. The crude mixture was purified by column chromatography (silica gel, gradient elution, 0 to 20% EtOAc/hexanes) to provide Intermediate A2.

### Intermediate A2-1: 4-(4-(2,3-dihydrobenzo[b] [1,4]dioxin-6-yl)indolin-1-yl)-2,6-dimethoxybenzaldehyde

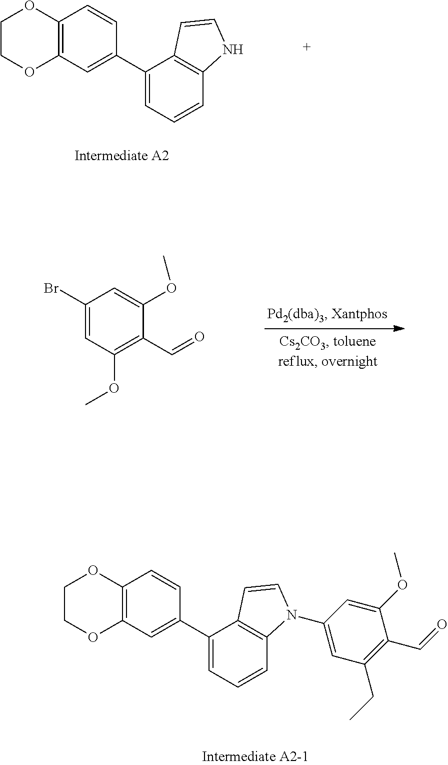 [SMILES: c1cc(-c2ccc3c(c2)OCCO3)c2cc[nH]c2c1 ; COc1cc(Br)cc(OC)c1C=O ; CCc1cc(-n2ccc3c(-c4ccc5c(c4)OCCO5)cccc32)cc(OC)c1C=O]

A mixture of Intermediate A2 (0.30 g, 1.19 mmol), 4-bromo-2,6-dimethoxybenzaldehyde (0.32 g, 1.31 mmol), Pd₂(dba)₃ (0.06 g, 0.06 mmol), Xantphos (0.07 g, 0.12 mmol) and Cs₂CO₃ (1.36 g, 4.19 mmol) in toluene 10 mL was sparged with argon for 10 min before it was refluxed under argon atmosphere for overnight. Then, the mixture was cooled down and diluted with water. The resulting mixture was extracted 3 times with EtOAc and the organic layer was collected, dried over anhydrous Na₂SO₄, filtered, and evaporated to dryness. The crude mixture was purified by column chromatography (silica gel, gradient elution, 0 to 60% EtOAc/hexanes) to provide Intermediate A2-1.

### Intermediate B1: 4-(4-bromo-1H-indol-1-yl)-2,6-dimethoxybenzaldehyde

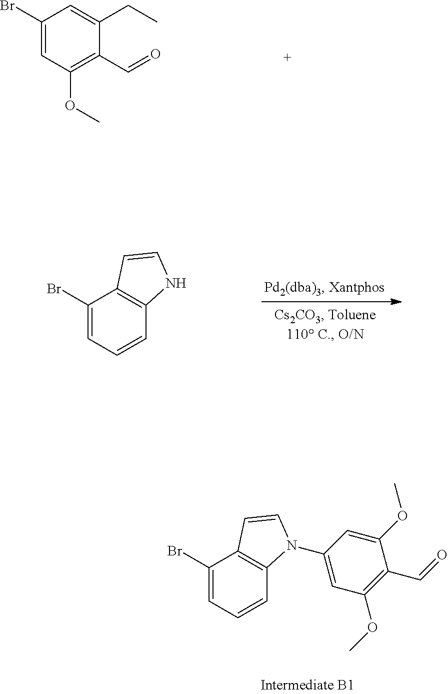 [SMILES: CCc1cc(Br)cc(OC)c1C=O ; Brc1cccc2[nH]ccc12 ; COc1cc(-n2ccc3c(Br)cccc32)cc(OC)c1C=O]

A mixture of 4-bromo-2,6-dimethoxybenzaldehyde (750 mg, 3.1 mmol), 4-bromo-1H-indole (500 mg, 2.6 mmol), Xantphos (150.00 mg, 0.26 mmol), Pd₂(dba)₃ (119.00 mg, 0.13 mmol) and Cs₂CO₃ (2.96 g, 9.1 mmol) was suspended in toluene. The resulting suspension was sparged with argon for 10 min. The reaction was sealed and stirred at 110° C. overnight under argon atmosphere, it was cooled down and diluted with brine. The resulting mixture was extracted 3 times with EtOAc and the organic layer was collected, dried over anhydrous Na₂SO₄, filtered, and evaporated to dryness. The crude mixture was purified by column chromatography (silica gel, gradient elution, 0 to 20% EtOAc/hexanes) to provide Intermediate B1.

### Intermediate B1-1: 2,6-dimethoxy-4-(4-(3,3,4,4-tetramethyl-13,2,5-bromodioxolane-1-yl)-1H-indol-1-yl)benzaldehyde

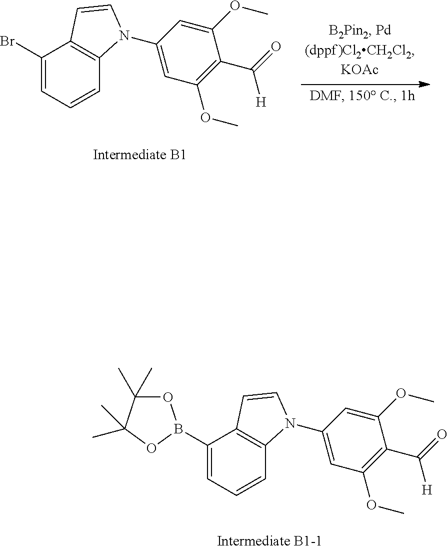 [SMILES: COc1cc(-n2ccc3c(Br)cccc32)cc(OC)c1C=O ; COc1cc(-n2ccc3c(B4OC(C)(C)C(C)(C)O4)cccc32)cc(OC)c1C=O]

Pd(dppf)Cl₂—CH₂Cl₂ (33 mg, 0.04 mmol) was added to a solution of Intermediate B1 (300.00 mg, 0.82 mmol), B₂Pin₂ (250.00 mg, 0.98 mmol), and KOAc (555.00 mg, 5.60 mmol) in DMF 25 mL. The resulting suspension was sparged with argon for 10 min. After the reaction was sealed and stirred at 150° C. for 1 hour under argon atmosphere, it was cooled down and diluted with water. The resulting mixture was extracted 3 times with EtOAc and the organic layer was collected, dried over anhydrous Na₂SO₄, filtered, and evaporated to dryness. The crude mixture was purified by column chromatography (silica gel, gradient elution, 10 to 20% EtOAc/hexanes) to provide Intermediate B1-1.

### Intermediate B1-2: tert-butyl-(R)-(4-(4-bromo-1H-indol-1-yl)-2,6-dimethoxybenzyl)((5-oxopyrrolidin-2-yl)methyl)carbamate

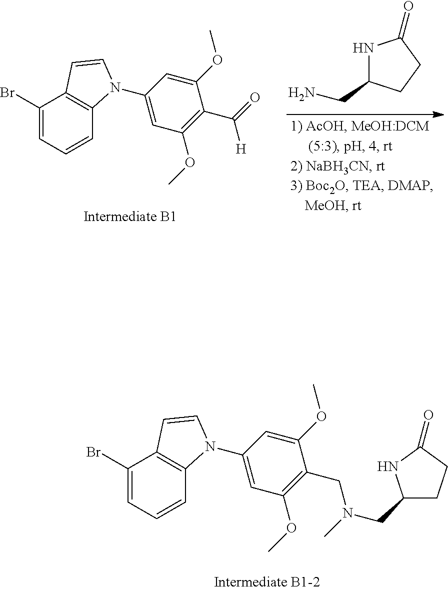 [SMILES: COc1cc(-n2ccc3c(Br)cccc32)cc(OC)c1C=O ; NC[C@@H]1CCC(=O)N1 ; COc1cc(-n2ccc3c(Br)cccc32)cc(OC)c1CN(C)C[C@@H]1CCC(=O)N1]

A mixture of Intermediate B1 (360.00 g, 1.00 mmol) and (S)-5-(aminomethyl)pyrrolidine-2-one (193.00 mg, 1.70 mmol) in MeOH:DCM (5:3, 50 mL) was stirred at room temperature and acetic acid was added until the pH was around 4, before sodium cyanoborohydride (565.00 mg, 5.00 mmol) was added. The reaction was stirred at room temperature for an additional 1 hour, quenched by adding a small amount of MeOH and evaporated to dryness. Then, the residue was dissolved in MeOH and di-tert-butyldicarbonate (261.00 mg, 1.20 mmol), triethylamine (180 mL, 1.3 mmol), and 4-dimethylaminopyridine (12.00 mg, 0.10 mmol) were added. After the reaction was stirred at room temperature for 2 h, water was added into the solution. The resulting mixture was extracted 3 times with EtOAc and the organic layer was collected, washed with brine, dried over anhydrous Na₂SO₄, filtered, and evaporated to dryness. The crude mixture was purified by column chromatography (silica gel, gradient elution, 0 to 10% Y, MeOH/DCM) to provide Intermediate B1-2.

### Intermediate B2: 4-(4-bromoindolin-1-yl)-2,6-dimethoxybenzaldehyde

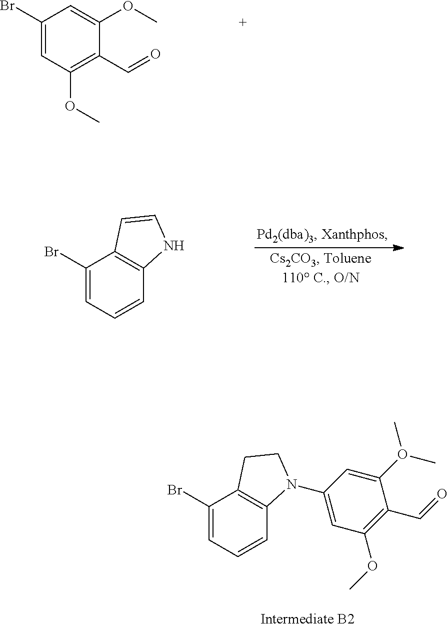

A mixture of 4-bromo-2,6-dimethoxybenzaldehyde (741.00 mg, 3.02 mmol), 4-bromoindoline (500.00 mg, 2.52 mmol), Xantphos (146.00 mg, 0.25 mmol), Pd₂(dba)₃ (115.00 mg, 0.13 mmol) and Cs₂CO₃ (2.87 g, 8.82 mmol) was processed till purification using similar procedures as described in Intermediate B1 resulting in Intermediate B2.

### Intermediate B3: 4-(4-bromo-1H-pyrrolo[2,3-b]pyridin-1-yl)-2,6-dimethoxybenzaldehyde

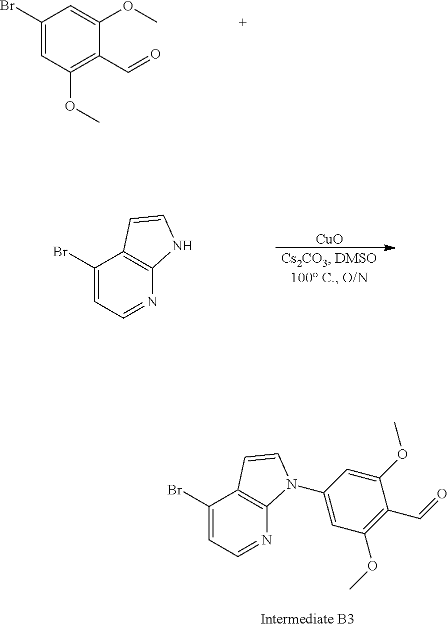 [SMILES: COc1cc(Br)cc(OC)c1C=O ; Brc1ccnc2[nH]ccc12 ; "CuO" ; COc1cc(-n2ccc3c(Br)ccnc32)cc(OC)c1C=O]

A mixture of 4-bromo-2,6-dimethoxybenzaldehyde (747.00 mg, 3.10 mmol), 4-Bromo-1H-indazole (500.00 mg, 2.54 mmol), CuO (10.00 mg, 0.127 mmol), and Cs₂CO₃ (1.66 g, 9.10 mmol) was suspended in DMSO. The resulting suspension was processed using similar procedures as described in Intermediate B1 to yield a crude mixture, which was purified by column chromatography (silica gel, gradient elution, 0 to 20% EtOAc/hexanes) to provide Intermediate B3.

### Intermediate B3-1: 2,6-dimethoxy-4-(4-(3,3,4,4-tetramethyl-1l3,2,5-bromodioxolan-1-yl)-2,3-dihydro-1H-pyrrolo[2,3-b]pyridin-1-yl)benzaldehyde

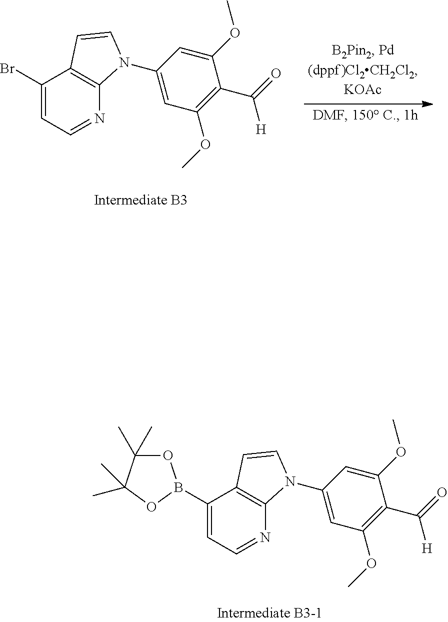 [SMILES: COc1cc(-n2ccc3c(Br)ccnc32)cc(OC)c1C=O ; COc1cc(-n2ccc3c(B4OC(C)(C)C(C)(C)O4)ccnc32)cc(OC)c1C=O]

Pd(dppf)Cl₂ CH₂C₂ (33 mg, 0.04 mmol) was added to a solution of Intermediate B3 (300.00 mg, 0.82 mmol), B₂Pin₂ (250.00 mg, 0.98 mmol), and KOAc (555.00 mg, 5.60 mmol) in

### Intermediate B4: 4-(4-bromo-1H-indazol-1-yl)-2,6-dimethoxybenzaldehyde

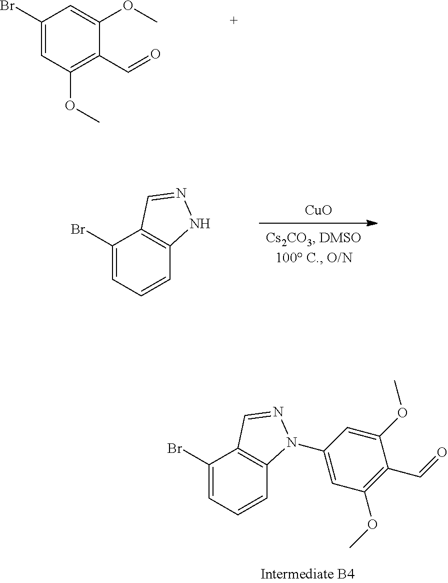 [SMILES: COc1cc(Br)cc(OC)c1C=O ; Brc1cccc2[nH]ncc12 ; "CuO" ; COc1cc(-n2ncc3c(Br)cccc32)cc(OC)c1C=O]

A mixture of 4-bromo-2,6-dimethoxybenzaldehyde (747.00 mg, 3.10 mmol), 4-Bromo-1H-indazole (500.00 mg, 2.54 mmol), CuO (10 mg, 0.13 mmol), and Cs₂CO₃ (1.66 g, 9.10 mmol) was processed till purification using similar procedures as described in Intermediate B1 to result in Intermediate B4.

### Intermediate B4-1: 2,6-dimethoxy-4-(4-(3,3,4,4-tetramethyl-1l3,2,5-bromadioxolan-1-yl)-1H-indazol-1-yl)benzaldehyde

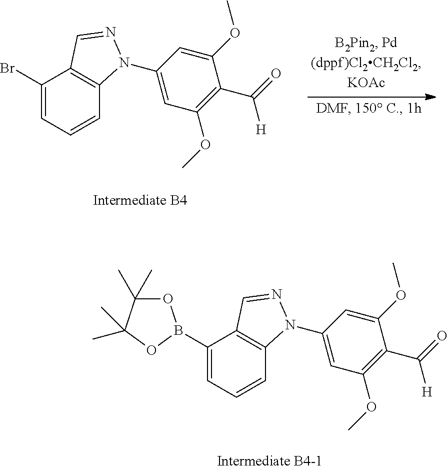 [SMILES: COc1cc(-n2ncc3c(Br)cccc32)cc(OC)c1C=O ; COc1cc(-n2ncc3c(B4OC(C)(C)C(C)(C)O4)cccc32)cc(OC)c1C=O]

Pd(dppf)Cl₂—CH₂Cl₂ (33 mg, 0.04 mmol) was added to a solution of Intermediate B4 (300.00 mg, 0.82 mmol), B₂Pin₂ (250.00 mg, 0.98 mmol), and KOAc (555.00 mg, 5.60 mmol) in DMF 25 mL. The resulting suspension was processed till purification using similar procedures as described in Intermediate B1-1 to result in Intermediate B4-1.

### Intermediate B4-2: tert-butyl (S)-(4-(4-bromo-1H-indazol-1-yl)-2,6-dimethoxybenzyl)((5-oxopyrrolidin-2-yl)methyl)carbamate

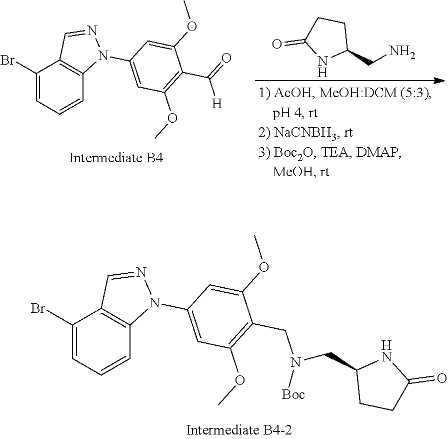 [SMILES: COc1cc(-n2ncc3c(Br)cccc32)cc(OC)c1C=O ; NC[C@@H]1CCC(=O)N1 ; COc1cc(-n2ncc3c(Br)cccc32)cc(OC)c1CN(C[C@@H]1CCC(=O)N1)C(=O)OC(C)(C)C]

A mixture of Intermediate B4 (360.00 g, 1.00 mmol) and (S)-5-(aminomethyl)pyrrolidine-2-one (193.00 mg, 1.70 mmol) in MeOH:DCM (5:3, 50 mL) was processed till purification using similar procedures as described in Intermediate B1-2 to result in Intermediate B4-2.

### Intermediate B4-β: (R)-1-(4-(4-bromo-1H-indazol-1-yl)-2,6-dimethoxybenzyl)pyrrolidin-3-ol

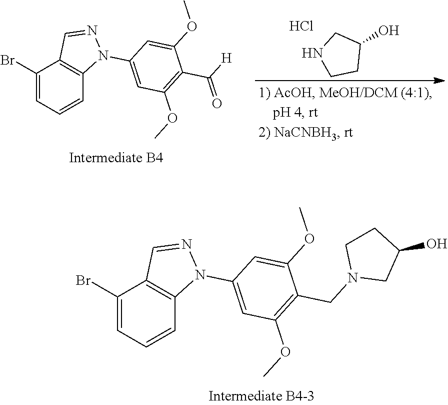 [SMILES: COc1cc(-n2ncc3c(Br)cccc32)cc(OC)c1C=O ; Cl ; O[C@@H]1CCNC1 ; COc1cc(-n2ncc3c(Br)cccc32)cc(OC)c1CN1CC[C@@H](O)C1]

NEt₃ was added to a solution of (3R)-pyrrolidin-3-ol hydrochloride (410.00 mg, 3.32 mmol) in 10 mL MeOH/DCM (1:1) until the pH was around 7. Then, the mixture was transferred to a mixture of Intermediate B4 (600.00 mg, 1.66 mmol) in 15 mL MeOH/DCM (1:1) at room temperature. After that, acetic acid was added until the pH was around 4 before sodium cyanoborohydride (314.00 mg, 4.98 mmol) was added. The reaction was stirred at room temperature for an additional 1 hour, quenched by a small amount of MeOH and evaporated to dryness. The crude mixture was purified by column chromatography (silica gel, gradient elution, 0 to 20% MeOH/DCM) to provide Intermediate B4-3.

### Intermediate B4-4: 4-((4-(4-bromo-1H-indazol-1-yl)-2,6-dimethoxybenzyl)(methyl)amino)bicyclo[2.2.2]octane-1-carboxylic acid

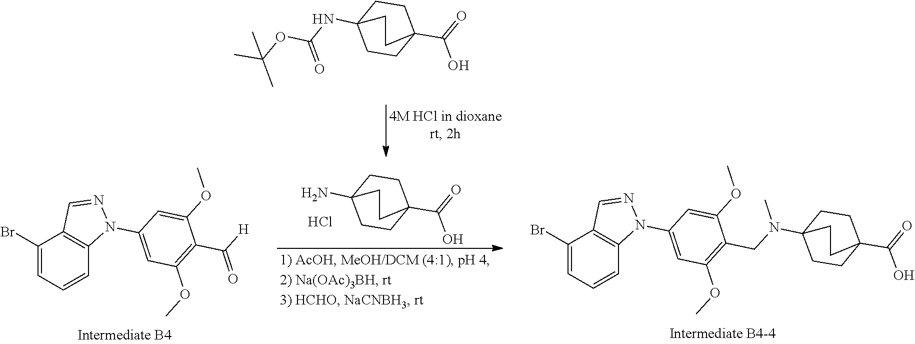 [SMILES: CC(C)(C)OC(=O)NC12CCC(C(=O)O)(CC1)CC2 ; COc1cc(-n2ncc3c(Br)cccc32)cc(OC)c1C=O ; Cl ; NC12CCC(C(=O)O)(CC1)CC2 ; COc1cc(-n2ncc3c(Br)cccc32)cc(OC)c1CN(C)C12CCC(C(=O)O)(CC1)CC2]

4-((tert-Butoxycarbonyl)amino)bicyclo[2.2.2]octane-1-carboxylic acid (500 mg, 1.86 mmol)) was added with 4M HCl in dioxane (5 mL). The mixture solution was stirred for 2 hours and evaporated to dryness. The crude mixture was used in the next step without purification. NEt₃ was added to a solution of 4-aminobicyclo[2.2.2]octane-1-carboxylic acid hydrochloride (342.00 mg, 1.66 mmol) in 5 mL of MeOH/DCM (1:1) until the pH was around 7. Then, the mixture was transferred to a mixture of Intermediate B4 (300 mg, 0.83 mmol) in 15 mL MeOH/DCM (1:1) at room temperature. After that, acetic acid was added until the pH was around 4 before sodium triacetoxyborohydride (880 mg, 4.15 mmol) was added. After the reaction was completed, 37% formaldehyde solution (1 mL) and sodium cyanoborohydride (51 mg, 0.82 mmol) were added, respectively. The complete reaction was quenched by adding a small amount of MeOH and evaporated to dryness. The crude mixture was purified by column chromatography (silica gel, gradient elution, 15 to 30% MeOH/DCM) to provide Intermediate B₄-4.

### Intermediate B4-5: trans-3-((4-(4-bromo-1H-indazol-1-yl)-2,6-dimethoxybenzyl)(methyl) amino)cyclobutan-1-ol

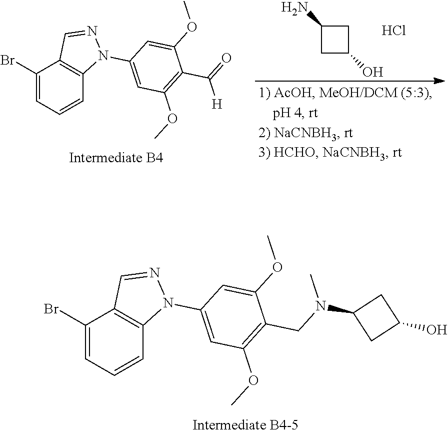 [SMILES: COc1cc(-n2ncc3c(Br)cccc32)cc(OC)c1C=O ; N[C@H]1C[C@H](O)C1 ; Cl ; COc1cc(-n2ncc3c(Br)cccc32)cc(OC)c1CN(C)[C@H]1C[C@H](O)C1]

NEt₃ was added to a solution of trans-aminocyclobutan-1-ol hydrochloride (236.0 mg, 1.91 mmol) in 5 mL MeOH/DCM (1:1) until the pH was around 7. Then, the mixture was transferred to a mixture of Intermediate B4 (0.370 g, 1.02 mmol) in 5 mL MeOH/DCM (1:1) at room temperature. After that, acetic acid was added until the pH was around 4, before sodium cyanoborohydride (200.00 mg, 3.18 mmol) was added. After the reaction was completed, 37% formaldehyde solution (I mL) and sodium cyanoborohydride (200.00 mg, 3.18 mmol) were added, respectively. The complete reaction was quenched by adding a small amount of MeOH and evaporated to dryness. The crude mixture was purified by column chromatography (silica gel, gradient elution, 0 to 30% MeOH/DCM) to provide Intermediate B4-5.

### Intermediate B5: 6-(4-bromo-1H-indazol-1-yl)-4-methoxynicotinaldehyde

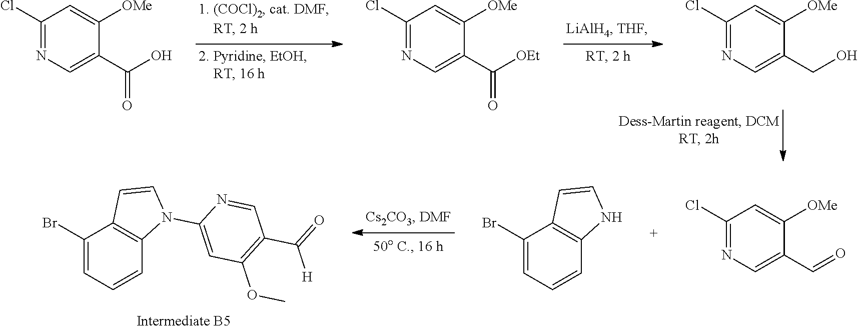 [SMILES: COc1cc(Cl)ncc1C(=O)O ; CCOC(=O)c1cnc(Cl)cc1OC ; COc1cc(Cl)ncc1CO ; COc1cc(-n2ccc3c(Br)cccc32)ncc1C=O ; Brc1cccc2[nH]ccc12 ; COc1cc(Cl)ncc1C=O]

6-chloro-4-methoxynicotinic acid (3.00 g, 16.0 mmol) was placed in a round-bottomed flask. The flask was cooled to 0° C. Oxalyl chloride (12.0 mL, 0.140 mol) was added to the reaction. Then, a few drops of DMF were added. The reaction mixture was stirred at room temperature for 2 h and dried by rotavapor to remove excess oxalyl chloride. The pyridine solution in EtOH (16% v/v) was slowly added to the crude acyl chloride from the previous step at 0° C. Then the reaction was left to reach room temperature and further stirred for 2 h, concentrated by rotavapor, diluted by EtOAc, and washed with saturated NaHCO₃ solution. The organic phase was dried by anhydrous Na₂SO₄. The solvent was removed to obtain the crude ester product for use in the next step without purification.

The crude product of ethyl 6-chloro-4-methoxynicotinate from the previous step (3.2 g, 14.9 mmol) was dissolved in THF. The solution was cooled to 0° C. Then, 2M lithium aluminum hydride in THF (15.2 mL, 30.4 mmol) was added dropwise to a cooled solution. The reaction was stirred at room temperature for 2 h before 2M NaOH solution was added, filtered and washed by EtOAc. The filtrate was collected and the solvent was removed to yield a crude alcohol product for use in the next step without purification.

The crude product of (6-chloro-4-methoxypyridin-3-yl)methanol (2.8 g, 16.2 mmol) from the previous step was dissolved in DCM. Dess-Martin reagent (7.5 g, 17.7 mmol) was added and the reaction was stirred at room temperature for 2 h. Saturated NaHCO₃ solution was added and the mixture was stirred at room temperature for 10 min. The crude mixture was extracted by DCM and purified by column chromatography (silica gel, gradient elution, 15 to 30% EtOAc/hexanes) to provide an aldehyde product for the next step.

6-chloro-4-methoxynicotinaldehyde (1.50 g, 8.77 mmol) was dissolved in DMF. Then, 4-bromo-1H-indazole (2.58 g, 13.1 mmol) and Cs₂CO₃ (5.71 g, 17.5 mmol) were added. The reaction mixture was heated to 50° C., stirred for 16 h, and diluted with water before extracted by EtOAc. The organic phase was washed with saturated NaCl, dried over anhydrous Na₂SO₄, and subsequently by rotavapor. The resulting crude mixture was purified by column chromatography (silica gel, gradient elution, 60 to 100% hexanes/DCM) to provide Intermediate B5

### Intermediate B5-1: 4-methoxy-6-(4-(4,4,5,5-tetramethyl-1,3,2-dioxaborolan-2-yl)-1H-indazol-1-yl)nicotinaldehyde

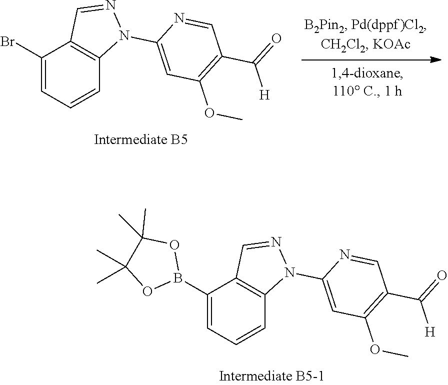 [SMILES: COc1cc(-n2ncc3c(Br)cccc32)ncc1C=O ; COc1cc(-n2ncc3c(B4OC(C)(C)C(C)(C)O4)cccc32)ncc1C=O]

Pd(dppf)Cl₂—CH₂Cl₂ (36.6 mg, 0.045 mmol) was added to a solution of Intermediate B5 (149.00 mg, 0.449 mmol), B₂Pin₂ (125.00 mg, 0.492 mmol), and potassium acetate (132.00 mg, 1.35 mmol) in 1,4-dioxane (10.0 ml). The resulting suspension was processed by using similar procedures as described in Intermediate B1-1 to yield a crude mixture, which was used in the next step without purification.

### Intermediate B6: 6-(4-bromo-1H-indol-1-yl)-4-methoxynicotinaldehyde

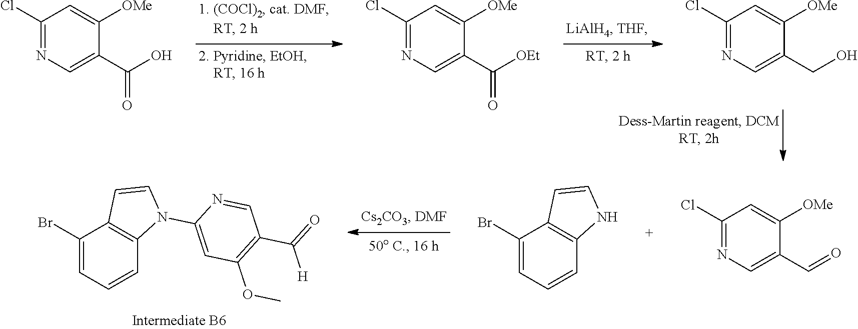 [SMILES: COc1cc(Cl)ncc1C(=O)O ; CCOC(=O)c1cnc(Cl)cc1OC ; COc1cc(Cl)ncc1CO ; COc1cc(-n2ccc3c(Br)cccc32)ncc1C=O ; Brc1cccc2[nH]ccc12 ; COc1cc(Cl)ncc1C=O]

This compound was prepared and purified by using similar procedures as described in the Intermediate B5 replacing 4-bromo-1H-indazole with 4-bromo-1H-indole, to obtain Intermediate B6.

### Intermediate B6-1: 4-methoxy-6-(4-(4,4,5,5-tetramethyl-1,3,2-dioxaborolan-2-yl)-1H-indol-1-yl)nicotinaldehyde

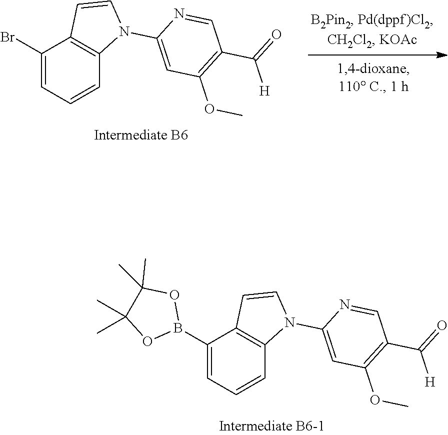 [SMILES: COc1cc(-n2ccc3c(Br)cccc32)ncc1C=O ; COc1cc(-n2ccc3c(B4OC(C)(C)C(C)(C)O4)cccc32)ncc1C=O]

Pd(dppf)Cl₂—CH₂Cl₂ (36.6 mg, 0.045 mmol) was added to a solution of Intermediate B6 (149.00 mg, 0.450 mmol), B₂Pin₂ (125.00 mg, 0.492 mmol), and potassium acetate (132.00 mg, 1.35 mmol) in 1,4-dioxane (10 mL). The resulting suspension was processed using similar procedures as described in Intermediate B1-1 to yield a crude mixture, which was used in the next step without purification.

### Intermediate B7: 4-(7-bromo-1-methyl-1H-indazol-3-yl)-2,6-dimethoxybenzaldehyde

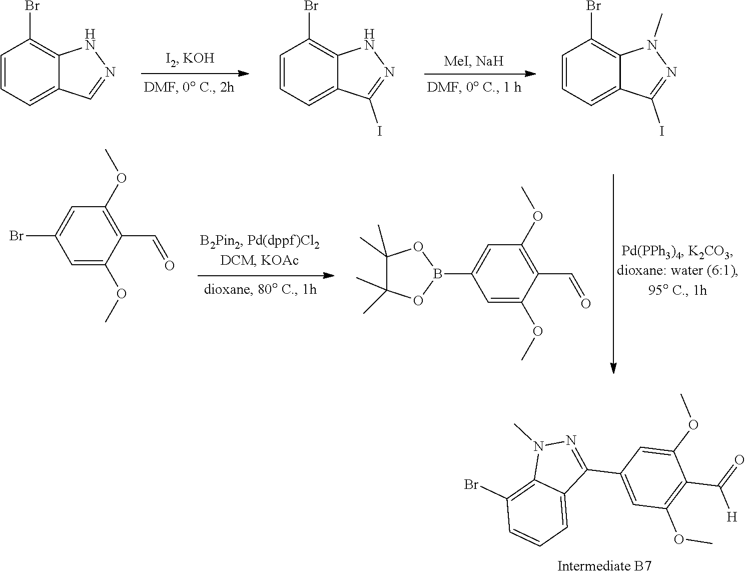 [SMILES: Brc1cccc2cn[nH]c12 ; Brc1cccc2c(I)n[nH]c12 ; Cn1nc(I)c2cccc(Br)c21 ; COc1cc(Br)cc(OC)c1C=O ; COc1cc(B2OC(C)(C)C(C)(C)O2)cc(OC)c1C=O ; COc1cc(-c2nn(C)c3c(Br)cccc23)cc(OC)c1C=O]

Potassium hydroxide (0.27 g, 4.84 mmol) was added to a solution of 7-bromo-1H-indazole (0.50 g, 2.42 mmol) in DMF (5 mL). After the solution was clear, the reaction was cooled to 0° C. and an iodine solution in DMF (2 mL) (368 mg, 2.90 mmol) was added to the solution. After stirring for 2 h, the reaction was quenched with sodium thiosulfate. The resulting mixture was extracted three times with EtOAc and the organic layer was collected, dried over anhydrous Na₂SO₄, filtered, and evaporated to dryness to provide 7-bromo-3-iodo-1H-indazole as the product. The product was further used without purification.

NaH (0.16 mg, 6.5 mmol) was added into a crude solution of 7-bromo-3-iodo-1H-indazole in DMF (4 mL). After stirring at room temperature for one hour, the solution was cooled in an ice bath. Methyl iodide (0.20 mL, 3.2 mmol) was added into the solution and the reaction was stirred for 30 min. The reaction was quenched by water and extracted with EtOAc. The organic layer was collected, dried over anhydrous Na₂SO₄, filtered, and evaporated to dryness. The product was further used without purification. The crude mixture was purified by column chromatography (silica gel, gradient elution, 10 to 20% EtOAc/hexanes) to provide 7-bromo-3-iodo-1-methyl-1H-indazole as the product.

A mixture of 4-bromo-2,6-dimethoxybenzaldehyde (0.91 g, 3.70 mmol), bis(pinacolato)diboron (1.13 g, 4.45 mmol), KOAc (2.51 g, 25.56 mmol), Pd(dppf)Cl₂·DCM (0.15 g, 0.19 mmol) was suspended in 50 mL of dioxane. The resulting suspension was purged with argon for 10 min. After the reaction was sealed and stirred at 80° C. for 1 hour under argon atmosphere, it was cooled down and diluted with water. The resulting mixture was extracted three times with EtOAc and the organic layer was collected, dried over anhydrous Na₂SO₄, filtered, and evaporated to dryness to provide 2,6-dimethoxy-4-(4,4,5,5-tetramethyl-1,3,2-dioxaborolan-2-yl)benzaldehyde as the product. The product was further used without purification.

A mixture of 7-bromo-3-iodo-1-methyl-1H-indazole (0.25 g, 0.74 mmol), 2,6-dimethoxy-4-(4,4,5,5-tetramethyl-1,3,2-dioxaborolan-2-yl)benzaldehyde (0.432 g, 1.48 mmol), Pd(PPh₃)₄ (0.08 g, 0.07 mmol) and K₂CO₃ (1.15 g, 8.2 mmol) in a mixture of 1,4-dioxane and H₂O (6:1) 10 mL was purged with argon for 10 min before stirring at 95° C. for 30 min under argon atmosphere. Then, the mixture was cooled and diluted with brine. The resulting mixture was extracted three times with EtOAc and the organic layer was collected, dried over anhydrous Na₂SO₄, filtered, and evaporated to dryness. The crude mixture was purified by column chromatography (silica gel, gradient elution, 20 to 50% EtOAc/hexanes) to provide the Intermediate B7.

### Intermediate C1: 6-(3-bromo-2-chlorophenyl)-2-methoxynicotinaldelhyde

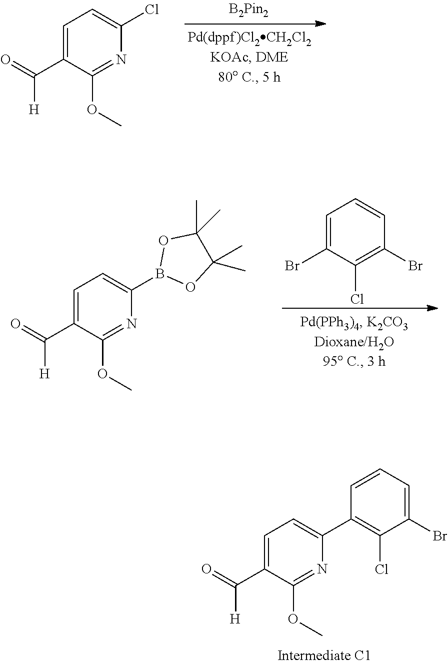 [SMILES: COc1nc(Cl)ccc1C=O ; COc1nc(B2OC(C)(C)C(C)(C)O2)ccc1C=O ; Clc1c(Br)cccc1Br ; COc1nc(-c2cccc(Br)c2Cl)ccc1C=O]

Pd(dppf)Cl₂·CH₂Cl₂ (0.14 g, 0.18 mmol) was added to a solution of 6-Chloro-2-methoxynicotinaldehyde (0.3 g, 1.75 mmol), B₂Pin₂ (0.49 g, 1.92 mmol), and KOAc (0.52 g, 5.25 mmol) in 1,2-dimethoxyethane 20 mL. The resulting suspension was sparged with argon for 10 min. After the reaction was sealed and stirred at 80° C. for 5 h under argon atmosphere, it was cooled down and diluted with water. The resulting mixture was extracted 3 times with EtOAc and the organic layer was collected, dried over anhydrous Na₂SO₄, filtered, and evaporated to dryness. The desired product was further used without purification.

Pd(PPh₃)₄ (0.20 g, 0.18 mmol) was added to a solution of 1,3-dibromo-2-chlorobenzene (0.71 g, 2.63 mmol) and 2-methoxy-6-(4,4,5,5-tetramethyl-1,3,2-dioxaborolan-2-yl)nicotinaldehyde (0.46 g, 1.75 mmol) in 1,4-dioxane 12 mL. A solution of K₂CO₃ (0.72 g, 5.25 mmol) in H₂O 2 mL was added to the reaction mixture before it was sparged with argon for 10 min. After the reaction mixture was stirred at 110° C. for 3 h under argon atmosphere, it was cooled down and diluted with brine. The resulting mixture was extracted 3 times with EtOAc and the organic layer was collected, dried over anhydrous Na₂SO₄, filtered, and evaporated to dryness. The crude mixture was purified by column chromatography (silica gel, gradient elution, 0 to 20% EtOAc/hexanes) to provide Intermediate C1.

### Intermediate C1-1: tert-butyl (S)-((6-(3-bromo-2-chlorophenyl)-2-methoxypyridin-3-yl)methyl)((5-oxopyrrolidin-2-yl)methyl)carbamate

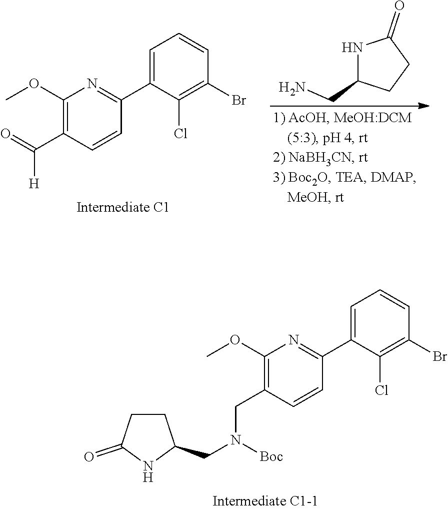 [SMILES: COc1nc(-c2cccc(Br)c2Cl)ccc1C=O ; NC[C@@H]1CCC(=O)N1 ; COc1nc(-c2cccc(Br)c2Cl)ccc1CN(C[C@@H]1CCC(=O)N1)C(=O)OC(C)(C)C]

A mixture of Intermediate C1 (1 g, 3.06 mmol) and (S)-5-(aminomethyl)pyrrolidine-2-one (0.42 g, 3.67 mmol) in MeOH:DCM (5:3, 10 mL) was stirred at room temperature and acetic acid was added until the pH was around 4, before sodium cyanoborohydride (0.96 g, 15.32 mmol) was added. The reaction was stirred at room temperature for an additional 1 hour, quenched by a small amount of MeOH and evaporated to dryness. Then, the residue was dissolved in MeOH and di-tert-butyldicarbonate (0.80 g, 3.67 mmol), triethylamine (0.55 mL, 3.98 mmol), and 4-dimethylaminopyridine (0.04 g, 0.31 mmol) were added. After the reaction was stirred at room temperature for 2 h, water was added into the solution. The resulting mixture was extracted 3 times with EtOAc and the organic layer was collected, washed with brine, dried over anhydrous Na₂SO₄, filtered, and evaporated to dryness. The crude mixture was purified by column chromatography (silica gel, gradient elution, 0 to 10% MeOH/DCM) to provide Intermediate C1-1.

### Intermediate C1-2: 1-((6-(3-bromo-2-chlorophenyl)-2-methoxypyridin-3-yl)methyl)-3-methylazetidin-3-ol

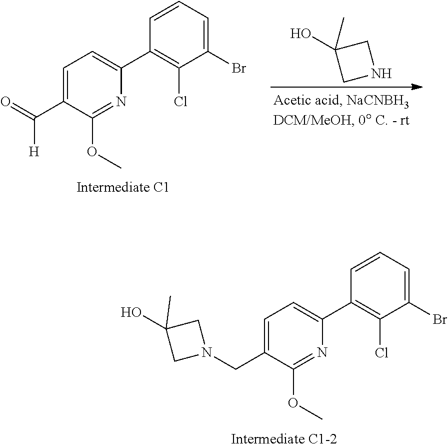

A mixture of Intermediate C1 (0.15 g, 0.46 mmol) and 3-methylazetidin-3-ol hydrochloride (0.11 g, 0.92 mmol) in MeOH:DCM (5:3, 10 mL) was stirred at room temperature and acetic acid was added until the pH was around 4. After the reaction was stirred for 1 hour, sodium cyanoborohydride (0.15 g, 2.30 mmol) was added. The reaction was stirred at room temperature for an additional 1 hour, quenched by a small amount of MeOH and evaporated to dryness. The crude mixture was purified by Sephadex column chromatography (100% MeOH) to provide Intermediate C1-2.

### Intermediate C1-3: 1-((6-(3-bromo-2-chlorophenyl)-2-methoxypyridin-3-yl)methyl)-3-methylazetidine-3-carboxylic acid

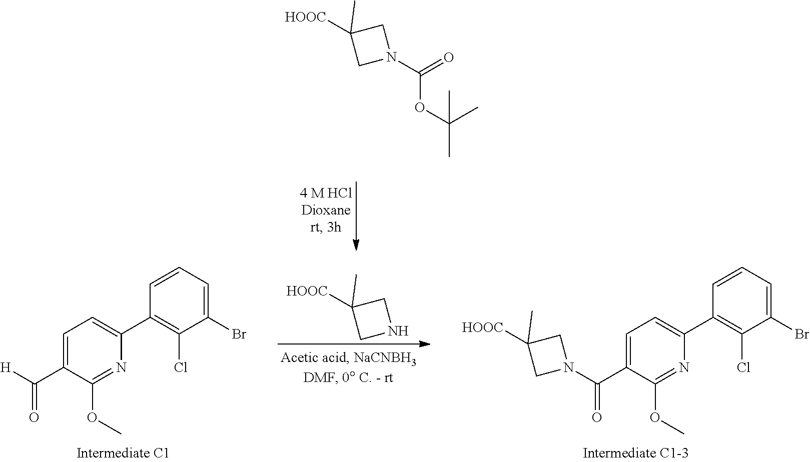

A solution of 1-(tert-butoxycarbonyl)azetidine-3-carboxylic acid in dioxane was added with 4M HCl and stirred at room temperature for 3 hours. The solution was concentrated under reduced pressure to afford azetidine-3-carboxylic acid hydrochloride.

A mixture of Intermediate C1 (0.15 g, 0.46 mmol) and 3-methylazetidine-3-carboxylic acid hydrochloride (0.14 g, 0.92 mmol) in DMF (10 mL) was stirred at room temperature and acetic acid was added until the pH was around 4. After the reaction was stirred for 1 hour, sodium cyanoborohydride (0.15 g, 2.30 mmol) was added. The reaction was stirred at room temperature for an additional 1 hour, quenched by a small amount of MeOH and evaporated to dryness. The crude mixture was purified by Sephadex column chromatography (100% MeOH) to provide Intermediate C1-3.

### Intermediate C1-4: (R)-1-((6-(3-bromo-2-chlorophenyl)-2-methoxypyridin-3-yl)methyl)pyrrolidin-3-ol

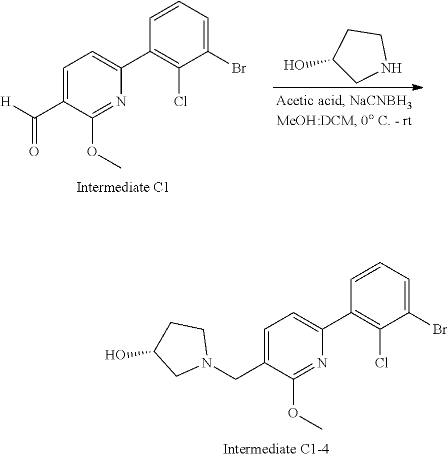 [SMILES: COc1nc(-c2cccc(Br)c2Cl)ccc1C=O ; O[C@@H]1CCNC1 ; COc1nc(-c2cccc(Br)c2Cl)ccc1CN1CC[C@@H](O)C1]

A mixture of Intermediate C1 (0.15 g, 0.46 mmol) and (R)-pyrrolidin-3-ol hydrochloride (0.12 g, 0.92 mmol) in MeOH:DCM (5:3, 10 mL) was stirred at room temperature and acetic acid was added until the pH was around 4. After the reaction was stirred for 1 hour, sodium cyanoborohydride (0.15 g, 2.30 mmol) was added. The reaction was stirred at room temperature for an additional 1 hour, quenched by a small amount of MeOH and evaporated to dryness. The crude mixture was purified by Sephadex column chromatography (100% MeOH) to provide Intermediate C1-4.

### Intermediate C1-5: tert-butyl ((6-(3-bromo-2-chlorophenyl)-2-methoxypyridin-3-yl)methyl)((cis-3-hydroxycyclobutyl)methyl)carbamate

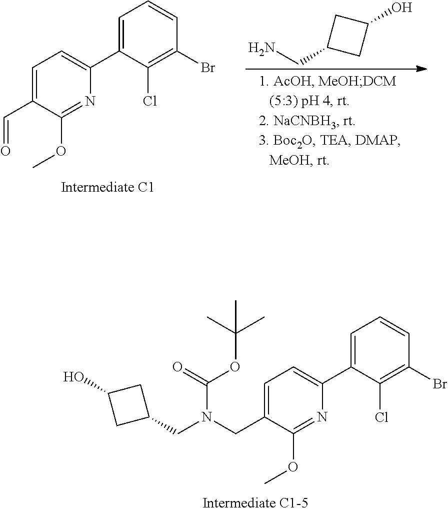 [SMILES: COc1nc(-c2cccc(Br)c2Cl)ccc1C=O ; NC[C@H]1C[C@@H](O)C1 ; COc1nc(-c2cccc(Br)c2Cl)ccc1CN(C[C@H]1C[C@@H](O)C1)C(=O)OC(C)(C)C]

A mixture of Intermediate C1 (1 g, 3.06 mmol) and cis-3-(aminomethyl)cyclobutanolone (0.37 g, 3.67 mmol) in MeOH:DCM (5:3, 10 mL) was stirred at room temperature and acetic acid was added until the pH was around 4. After the reaction was stirred for 1 hour, sodium cyanoborohydride (0.96 g, 15.32 mmol) was added. The reaction was stirred at room temperature for an additional 1 hour, quenched by a small amount of MeOH and evaporated to dryness. Then, the residue was dissolved in MeOH and di-tert-butyldicarbonate (0.80 g, 3.67 mmol), triethylamine (0.55 mL, 3.98 mmol), and 4-dimethylaminopyridine (0.04 g, 0.31 mmol) were added. After the reaction was stirred at room temperature for 2 h, water was added into the solution. The resulting mixture was extracted 3 times with EtOAc and the organic layer was collected, washed with brine, dried over anhydrous Na₂SO₄, filtered, and evaporated to dryness. The crude mixture was purified by column chromatography (silica gel, gradient elution, 0 to 10% MeOH/DCM) to provide Intermediate C1-5.

### Intermediate C1-6: 6-(3-bromo-2-chlorophenyl)-3-(4,5-dihydro-1H-imidazol-2-yl)-2-methoxypyridine

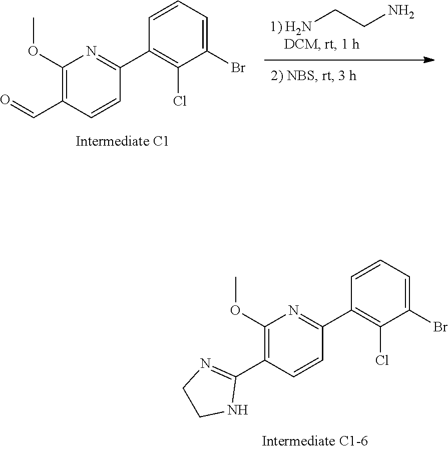

A solution of Intermediate C1 (0.20 g, 0.61 mmol) and ethylenediamine (0.10 mL, 1.50 mmol) in DCM 8 mL was stirred at rt for 1 hour before NBS (0.18 g, 1.01 mmol) was added in portion to this solution. After 3 hours, the resulting mixture was evaporated to dryness and purified by silica gel chromatography (0 to 30% MeOH/DCM). The fractions containing the product were collected and the solvent was removed under reduced pressure to provide Intermediate C1-6 (0.21 g). LCMS Method B, RT=3.375 min, m/z=366.0 [M+H]⁺, exact mass: 364.9931.

### Intermediate C1-7: 2-((6-(3-bromo-2-chlorophenyl)-2-methoxypyridin-3-yl)methyl)-2,6-diazaspiro[3,4]octan-7-one

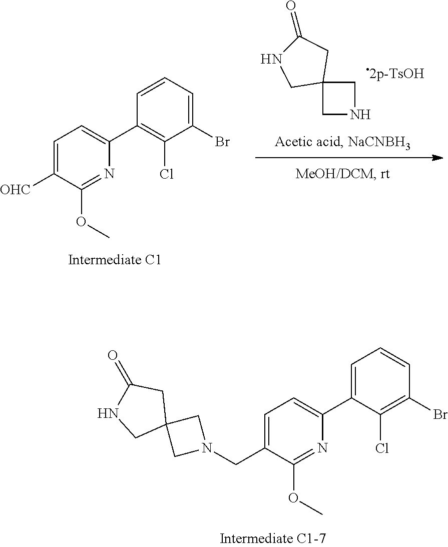 [SMILES: COc1nc(-c2cccc(Br)c2Cl)ccc1C=O ; O=C1CC2(CNC2)CN1 ; COc1nc(-c2cccc(Br)c2Cl)ccc1CN1CC2(CNC(=O)C2)C1]

A mixture of Intermediate C1 (0.60 g, 1.85 mmol) and 2,6-diazaspiro[3.4]octan-7-one bis(4-methylbenzenesulfonate) (1.04 g, 2.22 mmol) in 15 mL MeOH/DCM (1:1) was stirred at room temperature and acetic acid was added until the pH was around 4. After the reaction was stirred for 1 hour, sodium cyanoborohydride (0.58 g, 9.25 mmol) was added. The reaction was stirred at room temperature for an additional 2.5 hour, quenched by a small amount of MeOH and evaporated to dryness. The crude mixture was purified by column chromatography (silica gel, gradient elution, 3 to 15% MeOH/DCM) to provide Intermediate C1-7.

### Intermediate C1-8-1: cis-3-(((6-(3-bromo-2-chlorophenyl)-2-methoxypyridin-3-yl)methyl)amino)cyclobutanol

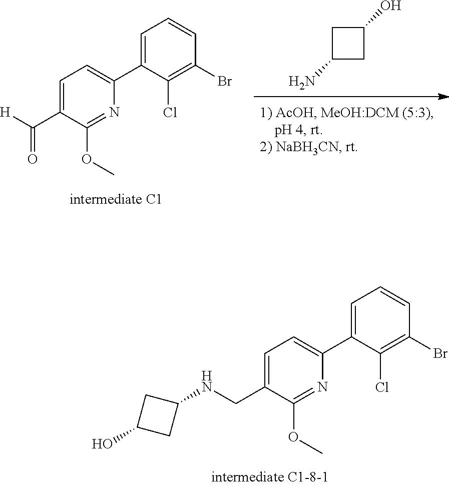 [SMILES: COc1nc(-c2cccc(Br)c2Cl)ccc1C=O ; N[C@H]1C[C@@H](O)C1 ; COc1nc(-c2cccc(Br)c2Cl)ccc1CN[C@H]1C[C@@H](O)C1]

A mixture of Intermediate C1 (1 g, 3.06 mmol) and cis-3-aminocyclobutanol (0.32 g, 3.67 mmol) in MeOH:DCM (5:3, 10 mL) was stirred at room temperature and acetic acid was added until the pH was ˜4. After the reaction was stirred for 1 hour, sodium cyanoborohydride (0.96 g, 15.32 mmol) was added. The reaction was stirred at room temperature for an additional 1 hour, quenched by a small amount of MeOH and evaporated to dryness. The crude mixture was purified by column chromatography (silica gel, gradient elution, 0 to 10% MeOH/DCM) to provide Intermediate C1-8-1.

### Intermediate C1-8: tert-butyl ((6-(3-bromo-2-chlorophenyl)-2-methoxypyridin-3-yl)methyl)((cis-3-hydroxycyclobutyl)carbamate

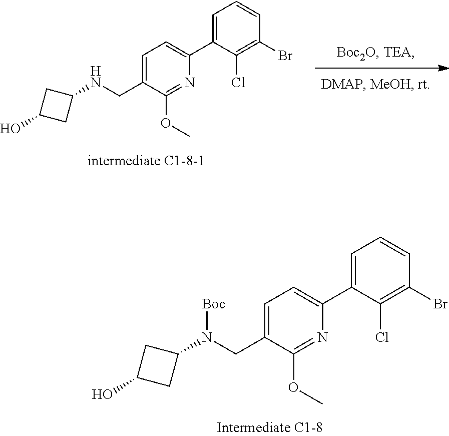 [SMILES: COc1nc(-c2cccc(Br)c2Cl)ccc1CN[C@H]1C[C@@H](O)C1 ; COc1nc(-c2cccc(Br)c2Cl)ccc1CN(C(=O)OC(C)(C)C)[C@H]1C[C@@H](O)C1]

Intermediate C1-8-1 (1.21 g, 3.06 mmol) was dissolved in MeOH and di-tert-butyldicarbonate (0.80 g, 3.67 mmol), triethylamine (0.55 mL, 3.98 mmol), and 4-dimethylaminopyridine (0.04 g, 0.31 mmol) were added. After the reaction was stirred at room temperature for 2 h, water was added into the solution. The resulting mixture was extracted three times with EtOAc and the organic layer was collected, washed with brine, dried over anhydrous Na₂SO₄, filtered, and evaporated to dryness. The crude mixture was purified by column chromatography (silica gel, gradient elution, 0 to 10% MeOH/DCM) to provide Intermediate C₁₋₈.

### Intermediate C1-9: 1-((6-(3-bromo-2-chlorophenyl)-2-methoxypyridin-3-yl)methyl)-N-cyclopropyl-3-methylazetidine-3-carboxamide

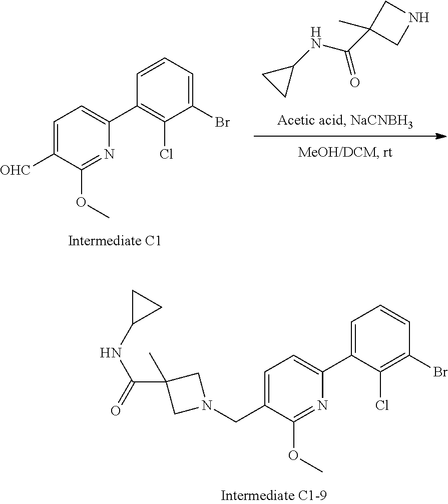 [SMILES: COc1nc(-c2cccc(Br)c2Cl)ccc1C=O ; CC1(C(=O)NC2CC2)CNC1 ; COc1nc(-c2cccc(Br)c2Cl)ccc1CN1CC(C)(C(=O)NC2CC2)C1]

### Step 1: N-cyclopropyl-3-methylazetidine-3-carboxamide

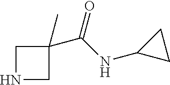

N, N-Diisopropylethylamine (2.0 mL, 11.6 mmol) was added into a mixture of 1-(tert-butoxycarbonyl)-3-methylazetidine-3-carboxylic acid (0.50 g, 2.30 mmol) and HATU (1.11 g, 2.91 mmol) in DMF. Then cyclopropanamine (0.11 g, 1.94 mmol) was added into a reaction and stirred at room temperature. After 16 h, the resulting mixture was extracted 3 times with EtOAc and the organic layer was collected, dried over anhydrous Na₂SO₄, filtered, and evaporated to dryness. The resulting oil was treated with 4M HCl in dioxane and stirred at room temperature for 30 min. After evaporation to dryness, the product was further used without purification.

### Step 2: 1-((6-(3-bromo-2-chlorophenyl)-2-methoxypyridin-3-yl)methyl)-N-cyclopropyl-3-methylazetidine-3-carboxamide

A mixture of Intermediate C1 (0.18 g, 0.55 mmol) and N-cyclopropyl-3-methylazetidine-3-carboxamide (0.10 g, 0.66 mmol) in 4 mL MeOH/DCM (1:1) was stirred at room temperature and acetic acid was added until the pH was ˜4. After the reaction was stirred for 1 hour, sodium cyanoborohydride (0.15 g, 2.30 mmol) was added. The reaction was stirred at room temperature for an additional 2.5 hours, quenched by a small amount of MeOH and evaporated to dryness. The crude mixture was purified by Sephadex column chromatography (100% MeOH) to provide Intermediate C1-9.

### Intermediate C1-10: 1-((6-(3-bromo-2-chlorophenyl)-2-methoxypyridin-3-yl)methyl)-3-methylazetidine-3-carboxamide

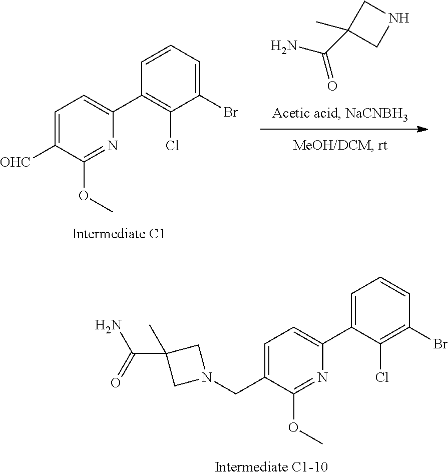

A mixture of Intermediate C1 (0.18 g, 0.55 mmol) and 3-methylazetidine-3-carboxamide (0.08 g, 0.66 mmol) in 4 mL MeOH/DCM (1:1) was stirred at room temperature and acetic acid was added until the pH was ˜4. After the reaction was stirred for 1 hour, sodium cyanoborohydride (0.15 g, 2.30 mmol) was added. The reaction was stirred at room temperature for an additional 2.5 hour, quenched by a small amount of MeOH and evaporated to dryness. The crude mixture was purified by Sephadex column chromatography (100% MeOH) to provide Intermediate C1-10.

### Intermediate C1-11: 2-((6-(3-bromo-2-chlorophenyl)-2-methoxypyridin-3-yl)methyl)-2,5-diazaspiro[3,4]octan-6-one

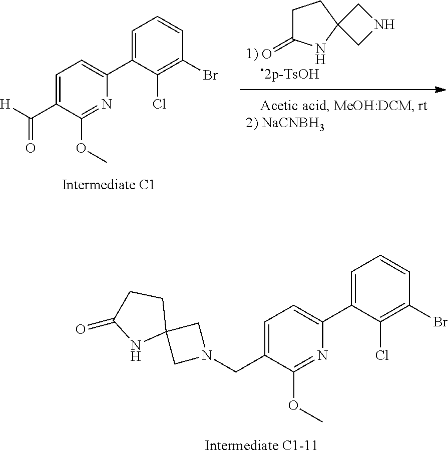 [SMILES: COc1nc(-c2cccc(Br)c2Cl)ccc1C=O ; O=C1CCC2(CNC2)N1 ; COc1nc(-c2cccc(Br)c2Cl)ccc1CN1CC2(CCC(=O)N2)C1]

NEt₃ was added to a mixture of 2,5-diazaspiro[3.4]octan-6-one bis(4-methylbenzenesulfonate) (0.61 g, 1.30 mmol) in 7 mL MeOH/DCM (1:1) until the pH was ˜7. Then, the mixture was transferred to Intermediate C1 (0.36 g, 1.10 mmol) at room temperature. Acetic acid was added until the pH was ˜4. After the reaction was stirred for 2 hours, sodium cyanoborohydride (0.33 g, 5.25 mmol) was added and the reaction was stirred at room temperature for overnight. The reaction mixture was purified by column chromatography (silica gel, gradient elution, 0 to 20% MeOH/DCM). The fractions containing the product were collected and the solvent was removed under reduced pressure to provide Intermediate C1-11.

### Intermediate C2: 6-(2-chloro-3-(4,4,5,5-tetramethyl-1,3,2-dioxaborolan-2-yl)phenyl)-2-methoxynicotinaldehyde

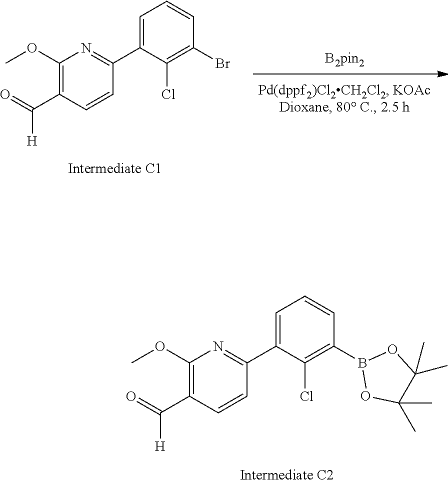 [SMILES: COc1nc(-c2cccc(Br)c2Cl)ccc1C=O ; COc1nc(-c2cccc(B3OC(C)(C)C(C)(C)O3)c2Cl)ccc1C=O]

Pd(dppf)Cl₂—CH₂Cl₂ (0.05 g, 0.06 mmol) was added to a solution of Intermediate C1 (0.2 g, 0.62 mmol), B₂Pin₂ (0.19 g, 0.74 mmol), and KOAc (0.18 g, 1.85 mmol) in 1,4-dioxane 10 mL. The resulting suspension was sparged with argon for 10 min. After the reaction was sealed and stirred at 80° C. for 2.5 h under argon atmosphere, it was cooled down and diluted with water. The resulting mixture was extracted 3 times with EtOAc and the organic layer was collected, dried over anhydrous Na₂SO₄, filtered, and evaporated to dryness. The Intermediate C₂ product was further used without purification.

### Intermediate D1: 6-(2-chloro-3-(1-(4-formyl-3,5-dimethoxyphenyl)-1H-indol-4-yl)phenyl)-2-methoxynicotinaldehyde

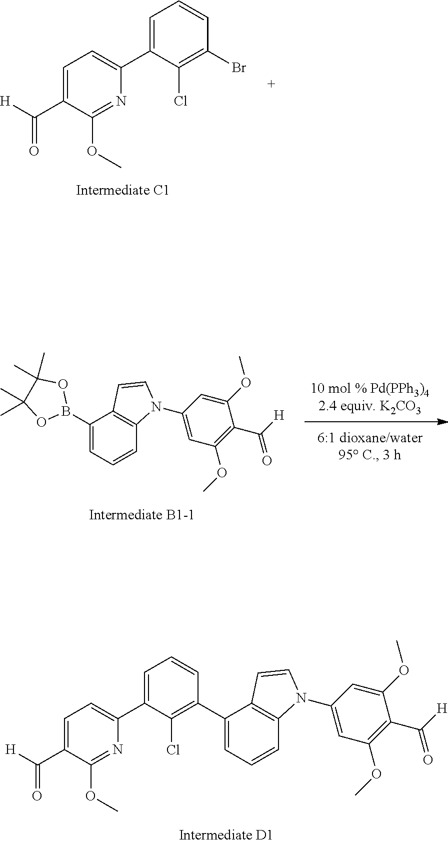 [SMILES: COc1nc(-c2cccc(Br)c2Cl)ccc1C=O ; COc1cc(-n2ccc3c(B4OC(C)(C)C(C)(C)O4)cccc32)cc(OC)c1C=O ; COc1cc(-n2ccc3c(-c4cccc(-c5ccc(C=O)c(OC)n5)c4Cl)cccc32)cc(OC)c1C=O]

A mixture of Intermediate C1 (126.00 mg, 0.39 mmol), Intermediate B1-1 (188.00 mg, 0.46 mmol), Pd(PPh₃)₄ (45.10 mg, 0.04 mmol) and K₂CO₃ (129.40 mg, 0.94 mmol) in a mixture of 1,4-dioxane and H₂O (6:1) 14 mL was sparged with argon for 10 min before stirring at 95° C. for 3 h under argon atmosphere. Then, the mixture was cooled down and diluted with brine. The resulting mixture was extracted 3 times with EtOAc and the organic layer was collected, dried over anhydrous Na₂SO₄, filtered, and evaporated to dryness. The crude product was purified by column chromatography (silica gel, gradient elution, 20 to 50% EtOAc/hexanes) to provide Intermediate D1.

### Intermediate D2: 6-(2-chloro-3-(1-(4-formyl-3,5-dimethoxyphenyl)-1H-indazol-4-yl)phenyl)-2-methoxynicotinaldehyde

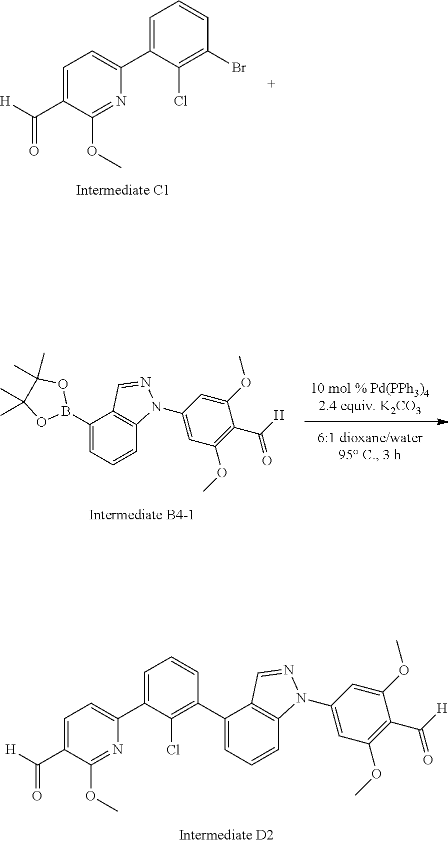 [SMILES: COc1nc(-c2cccc(Br)c2Cl)ccc1C=O ; COc1cc(-n2ncc3c(B4OC(C)(C)C(C)(C)O4)cccc32)cc(OC)c1C=O ; COc1cc(-n2ncc3c(-c4cccc(-c5ccc(C=O)c(OC)n5)c4Cl)cccc32)cc(OC)c1C=O]

Intermediate D2 was prepared by using similar procedures as described in Intermediate D1, replacing Intermediate B1-1 with Intermediate B4-1, to yield a crude product which was purified by column chromatography (silica gel, gradient elution, 20 to 80% EtOAc/hexanes). LCMS Method B, RT=4.747 min, m/z=528.2 [M+H]⁺, exact mass: 527.1248.

### Intermediate D3: 6-(2-chloro-3-(1-(5-formyl-4-methoxypyridin-2-yl)-1H-indazol-4-yl)phenyl)-2-methoxynicotinaldehyde

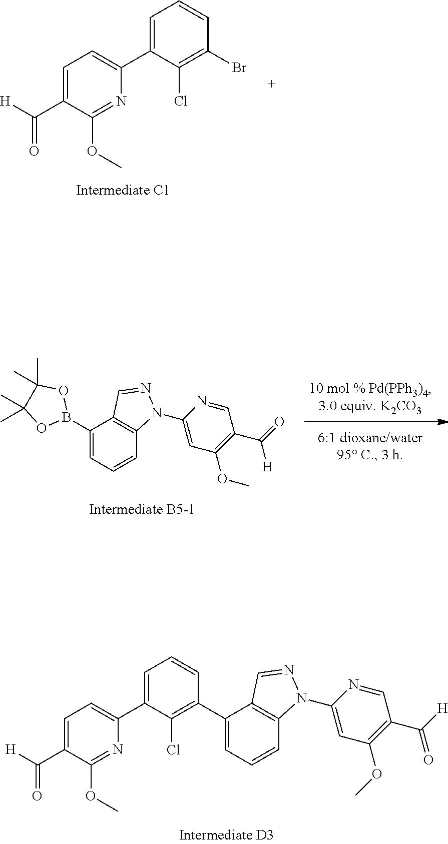 [SMILES: COc1nc(-c2cccc(Br)c2Cl)ccc1C=O ; COc1cc(-n2ncc3c(B4OC(C)(C)C(C)(C)O4)cccc32)ncc1C=O ; COc1cc(-n2ncc3c(-c4cccc(-c5ccc(C=O)c(OC)n5)c4Cl)cccc32)ncc1C=O]

Intermediate D3 was prepared by using similar procedures as described in Intermediate D1, replacing Intermediate B1-1 with Intermediate B5-1, to yield a crude product which was purified by column chromatography (silica gel, gradient elution, 10 to 60% EtOAc/hexanes).

### Intermediate D4: 6-(2-chloro-3-(1-(5-formyl-4-methoxypyridin-2-yl)-1H-indol-4-yl)phenyl)-2-methoxynicotinaldehyde

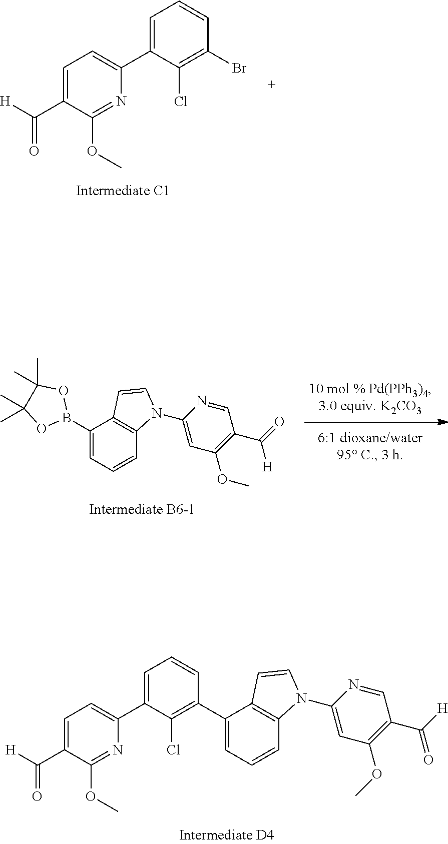 [SMILES: COc1nc(-c2cccc(Br)c2Cl)ccc1C=O ; COc1cc(-n2ccc3c(B4OC(C)(C)C(C)(C)O4)cccc32)ncc1C=O ; COc1cc(-n2ccc3c(-c4cccc(-c5ccc(C=O)c(OC)n5)c4Cl)cccc32)ncc1C=O]

Intermediate D4 was prepared by using similar procedures as described in Intermediate D1, replacing Intermediate B1-1 with Intermediate B6-1, to yield a crude product which was purified by column chromatography (silica gel, gradient elution, 10 to 60% EtOAc/hexanes).

### Intermediate E1: (S)-tert-butyl ((6-(2-chloro-3-(1-(4-formyl-3,5-dimethoxyphenyl)-1H-indol-4-yl)phenyl)-2-methoxypyridin-3-yl)methyl)((5-oxopyrrolidin-2-yl)methyl)carbamate

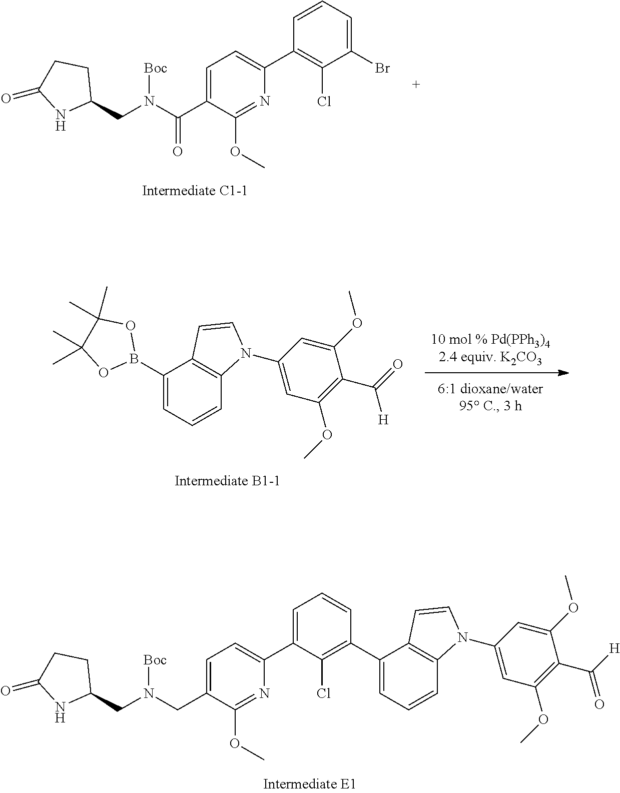 [SMILES: COc1nc(-c2cccc(Br)c2Cl)ccc1C(=O)N(C[C@@H]1CCC(=O)N1)C(=O)OC(C)(C)C ; COc1cc(-n2ccc3c(B4OC(C)(C)C(C)(C)O4)cccc32)cc(OC)c1C=O ; COc1cc(-n2ccc3c(-c4cccc(-c5ccc(CN(C[C@@H]6CCC(=O)N6)C(=O)OC(C)(C)C)c(OC)n5)c4Cl)cccc32)cc(OC)c1C=O]

A reaction mixture of Intermediate C1-1 and Intermediate B1-1 was processed by using similar procedures as described in Intermediate D1 to yield a crude mixture, which was then purified by column chromatography (silica gel, gradient elution, 50 to 100% EtOAc/hexanes) to provide Intermediate E1.

### Intermediate E2: (S)-tert-butyl ((6-(2-chloro-3-(1-(4-formyl-3,5-dimethoxyphenyl)-1H-pyrrolo[2,3-b]pyridin-4-yl)phenyl)-2-methoxypyridin-3-yl)methyl)((5-oxopyrrolidin-2-yl)methyl)carbamate

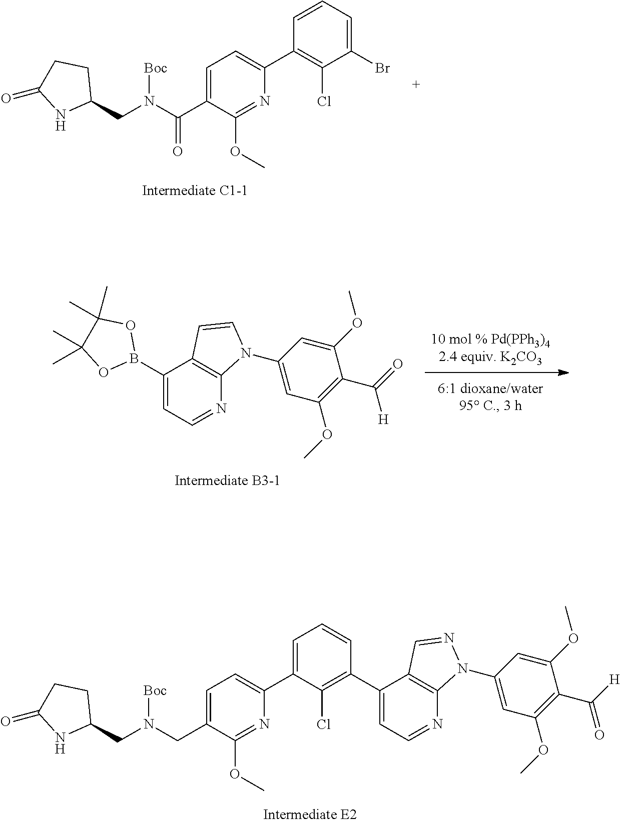 [SMILES: COc1nc(-c2cccc(Br)c2Cl)ccc1C(=O)N(C[C@@H]1CCC(=O)N1)C(=O)OC(C)(C)C ; COc1cc(-n2ccc3c(B4OC(C)(C)C(C)(C)O4)ccnc32)cc(OC)c1C=O ; COc1cc(-n2ncc3c(-c4cccc(-c5ccc(CN(C[C@@H]6CCC(=O)N6)C(=O)OC(C)(C)C)c(OC)n5)c4Cl)ccnc32)cc(OC)c1C=O]

A reaction mixture of Intermediate C1-1 and Intermediate B3-1 was processed by using similar procedures as described in Intermediate D1 to yield a crude mixture, which was then purified by column chromatography (silica gel, gradient elution, 50 to 100% EtOAc/hexanes) to provide Intermediate E2.

### Intermediate E3: (S)-tert-butyl ((6-(2-chloro-3-(1-(4-formyl-3,5-dimethoxyphenyl)-1H-indazol-4-yl)phenyl)-2-methoxypyridin-3-yl)methyl)((5-oxopyrrolidin-2-yl)methyl)carbamate

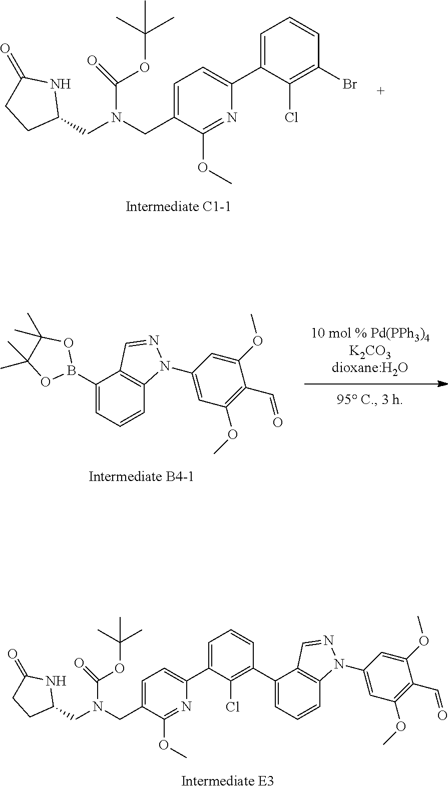 [SMILES: COc1nc(-c2cccc(Br)c2Cl)ccc1CN(C[C@@H]1CCC(=O)N1)C(=O)OC(C)(C)C ; COc1cc(-n2ncc3c(B4OC(C)(C)C(C)(C)O4)cccc32)cc(OC)c1C=O ; COc1cc(-n2ncc3c(-c4cccc(-c5ccc(CN(C[C@@H]6CCC(=O)N6)C(=O)OC(C)(C)C)c(OC)n5)c4Cl)cccc32)cc(OC)c1C=O]

A reaction mixture of Intermediate C1-1 and Intermediate B4-1 was processed by using similar procedures as described in Intermediate D1 to yield a crude mixture, which was then purified by column chromatography (silica gel, gradient elution, 50 to 100% EtOAc/hexanes) to provide Intermediate E3.

### Intermediate E4: tert-butyl ((6-(2-chloro-3-(1-(4-formyl-3,5-dimethoxyphenyl)-1H-indazol-4-yl)phenyl)-2-methoxypyridin-3-yl)methyl)((cis-3-hydroxycyclobutyl)methyl)carbamate

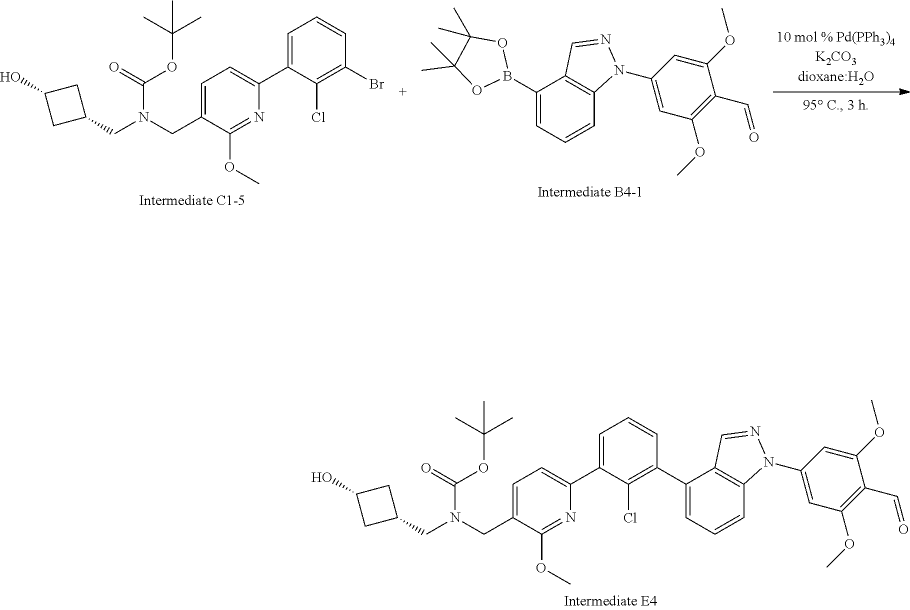 [SMILES: COc1nc(-c2cccc(Br)c2Cl)ccc1CN(C[C@H]1C[C@@H](O)C1)C(=O)OC(C)(C)C ; COc1cc(-n2ncc3c(B4OC(C)(C)C(C)(C)O4)cccc32)cc(OC)c1C=O ; COc1cc(-n2ncc3c(-c4cccc(-c5ccc(CN(C[C@H]6C[C@@H](O)C6)C(=O)OC(C)(C)C)c(OC)n5)c4Cl)cccc32)cc(OC)c1C=O]

A reaction mixture of Intermediate C1-5 and Intermediate B4-1 was processed by using similar procedures as described in Intermediate D1 to yield a crude mixture, which was then purified by column chromatography (silica gel, gradient elution, 50 to 100% EtOAc/hexanes) to provide Intermediate E4.

### Intermediates: 4-(4-(2-chloro-3-(5-(4,5-dihydro-1H-imidazol-2-yl)-6-methoxypyridin-2-yl)phenyl)-1H-indazol-1-yl)-2,6-dimethoxybenzaldehyde

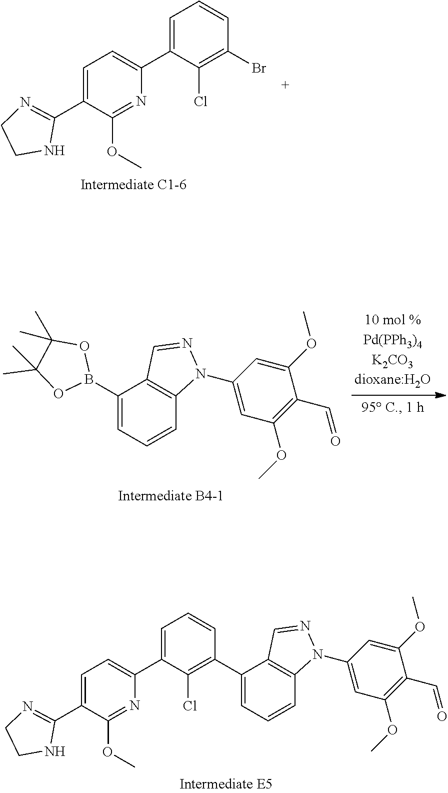 [SMILES: COc1nc(-c2cccc(Br)c2Cl)ccc1C1=NCCN1 ; COc1cc(-n2ncc3c(B4OC(C)(C)C(C)(C)O4)cccc32)cc(OC)c1C=O ; COc1cc(-n2ncc3c(-c4cccc(-c5ccc(C6=NCCN6)c(OC)n5)c4Cl)cccc32)cc(OC)c1C=O]

A reaction mixture of Intermediate C1-6 and Intermediate B4-1 was processed by using similar procedures as described in Intermediate D1 to yield a crude mixture, which was then purified by column chromatography (silica gel, gradient elution, 0 to 100% MeOH/DCM) to provide Intermediate E5. LCMS Method B, RT=3.740 min, m/z=568.2 [M+H]⁺, exact mass. 567.1673.

### Intermediate E6: 4-(4-(2-chloro-3-(6-methoxy-5-((7-oxo-2,6-diazaspiro[3,4]octan-2-yl)methyl)pyridin-2-yl)phenyl)-1H-indazol-1-yl)-2,6-dimethoxybenzaldehyde

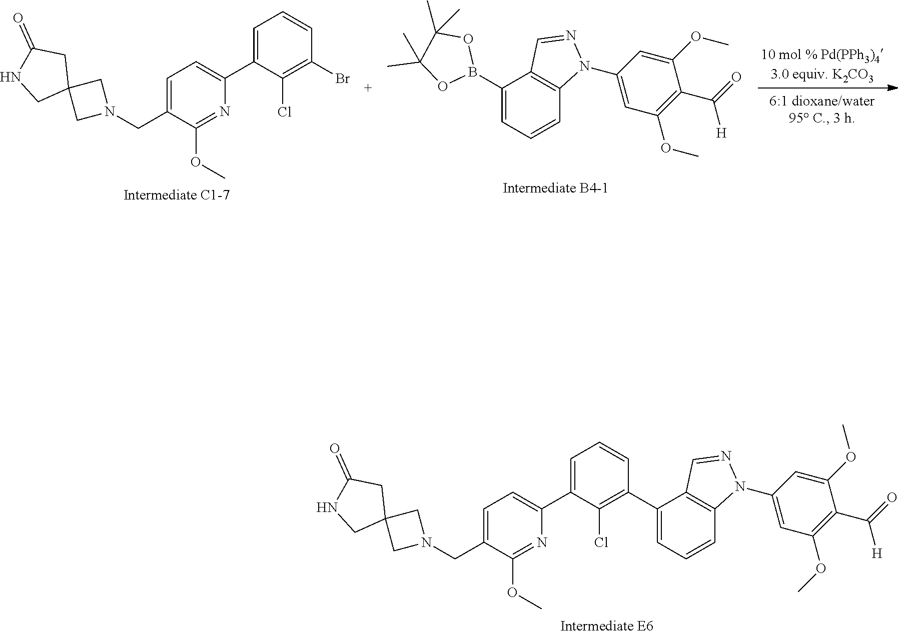 [SMILES: COc1nc(-c2cccc(Br)c2Cl)ccc1CN1CC2(CNC(=O)C2)C1 ; COc1cc(-n2ncc3c(B4OC(C)(C)C(C)(C)O4)cccc32)cc(OC)c1C=O ; COc1cc(-n2ncc3c(-c4cccc(-c5ccc(CN6CC7(CNC(=O)C7)C6)c(OC)n5)c4Cl)cccc32)cc(OC)c1C=O]

A reaction mixture of Intermediate C1-7 and Intermediate B4-1 was processed by using similar procedures as described in Intermediate D1 to yield a crude mixture, which was then purified by column chromatography (silica gel, gradient elution, 0 to 15% MeOH/DCM) to provide Intermediate E6.

### Intermediate E7-1: 4-(4-(2-chloro-3-(5-(((cis-3-hydroxycyclobutyl)amino)methyl-6-methoxypyridin-2-yl)phenyl-1H-indazol-1-γ)-2,6-dimethoxybenzaldehyde

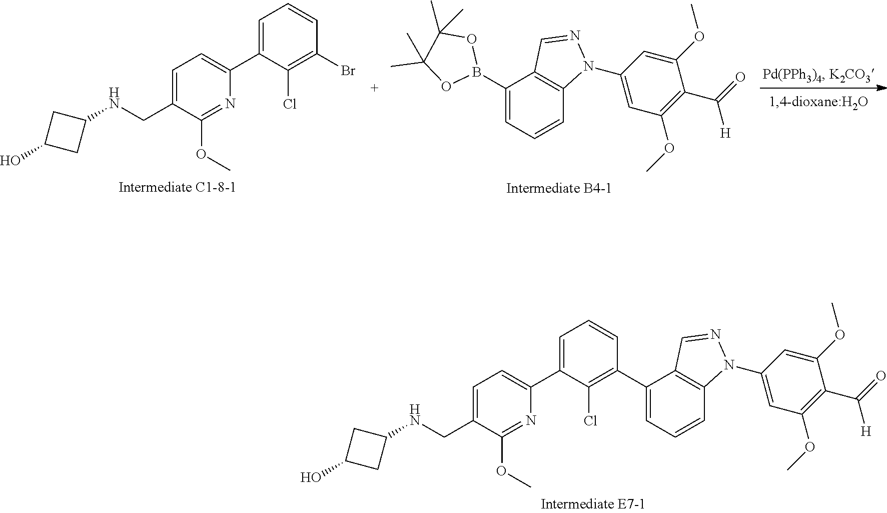

A reaction mixture of Intermediate C1-8-1 and Intermediate B4-1 was processed by using similar procedures as described in Intermediate D1 to yield a crude mixture, which was then purified by column chromatography (silica gel, gradient elution, 0 to 20% MeOH/DCM) to provide Intermediate E7-1.

### Intermediate E7. tert-butyl ((6-(2-chloro-3-(1-(4-formyl-3,5-dimethyloxyphenyl)-1H-indazol-4-yl)phenyl)-2-methoxypyridin-3-yl)methyl)(cis-3-hydroxycyclobutyl)carbamate

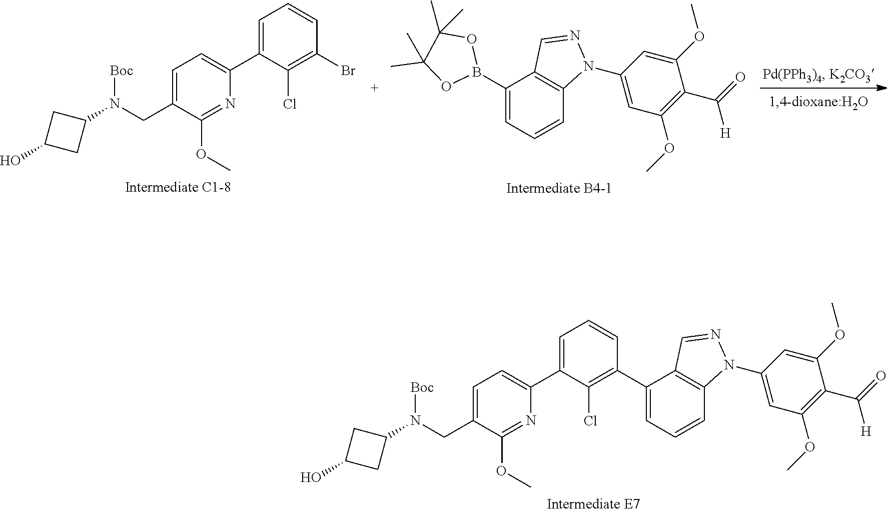

A reaction mixture of Intermediate C1-8 and Intermediate 1B₄-1 was processed by using similar procedures as described in Intermediate D1 to yield a crude mixture, which was then purified by column chromatography (silica gel, gradient elution, 0 to 15% MeOH/DCM) to provide Intermediate E7.

### Intermediate E8: 1-((6-(2-chloro-3-(1-(4-formyl-3,5-dimethoxyphenyl)-1H-indazol-4-yl)phenyl)-2-methoxypyridin-3-yl)methyl)-N-cyclopropyl-3-methylazetidine-3-carboxamide

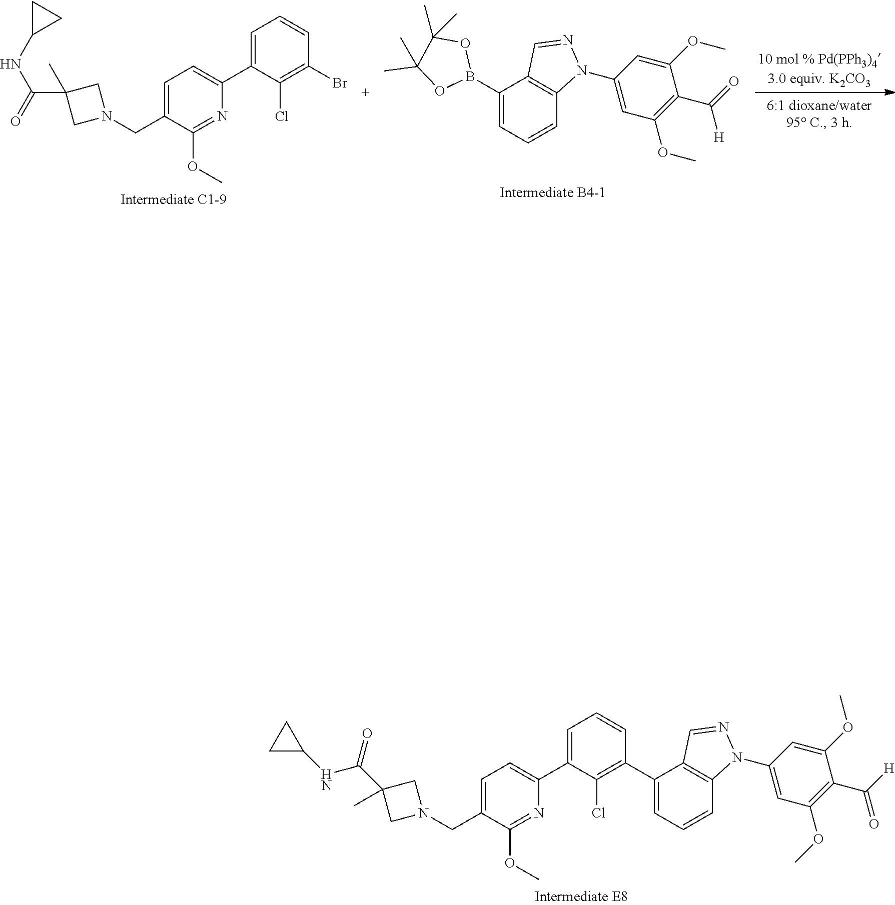 [SMILES: COc1nc(-c2cccc(Br)c2Cl)ccc1CN1CC(C)(C(=O)NC2CC2)C1 ; COc1cc(-n2ncc3c(B4OC(C)(C)C(C)(C)O4)cccc32)cc(OC)c1C=O ; COc1cc(-n2ncc3c(-c4cccc(-c5ccc(CN6CC(C)(C(=O)NC7CC7)C6)c(OC)n5)c4Cl)cccc32)cc(OC)c1C=O]

A reaction mixture of Intermediate C1-9 and Intermediate 1B₄-1 was processed by using similar procedures as described in Intermediate D1 to yield a crude mixture, which was then purified by column chromatography (silica gel, gradient elution, 0 to 10% MeOH/DCM) to provide Intermediate E8.

### Intermediate E9: 1-((6-(2-chloro-3-(1-(4 formyl-3,5-dimethoxyphenyl)-1H-indazol-4-yl)phenyl)-2-methoxypyridin-3-yl)methyl)-3-methylazetidine-3-carboxamide

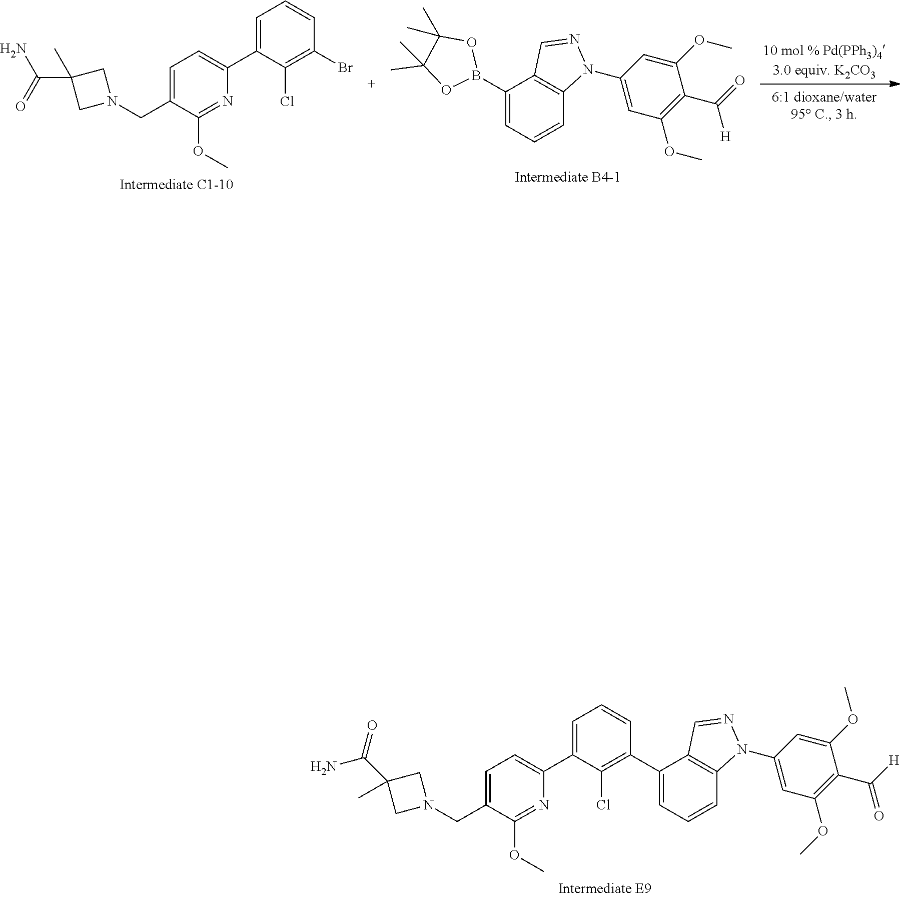 [SMILES: COc1nc(-c2cccc(Br)c2Cl)ccc1CN1CC(C)(C(N)=O)C1 ; COc1cc(-n2ncc3c(B4OC(C)(C)C(C)(C)O4)cccc32)cc(OC)c1C=O ; COc1cc(-n2ncc3c(-c4cccc(-c5ccc(CN6CC(C)(C(N)=O)C6)c(OC)n5)c4Cl)cccc32)cc(OC)c1C=O]

A reaction mixture of Intermediate C1-10 and Intermediate B4-1 was processed by using similar procedures as described in Intermediate D1 to yield a crude mixture, which was then purified by column chromatography (silica gel, gradient elution, 0 to 15% MeOH/DCM) to provide Intermediate E9.

### Intermediate E10: 4-(4-(2-chloro-3-(6-methoxy-5-((6-oxo-2,5-diazaspiro[3,4]octan-2-yl)methyl)pyridin-2-yl)phenyl)-1H-indazol-1-yl)-2,6-dimethoxybenzaldehyde

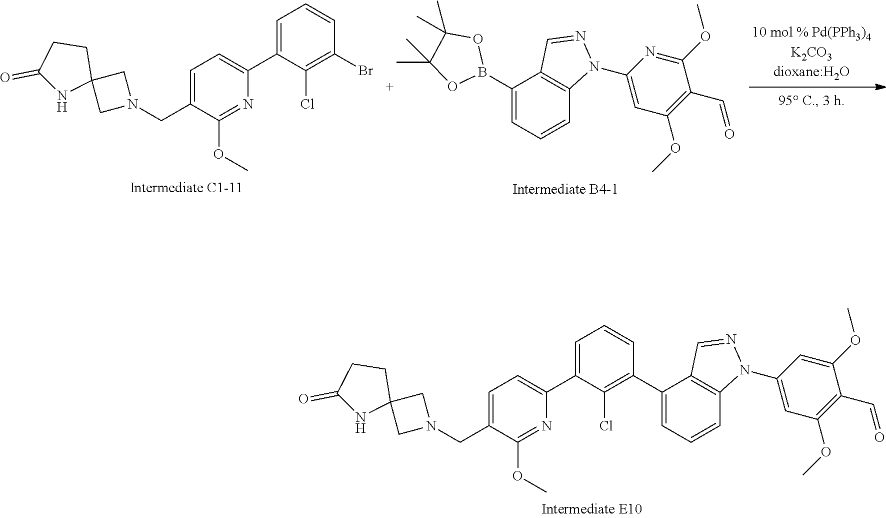

A reaction mixture of Intermediate C1-11 and Intermediate B4-1 was processed by using similar procedures as described in Intermediate D1 to yield a crude mixture, which was then purified by column chromatography (silica gel, gradient elution, 0 to 20% MeOH/DCM) to provide Intermediate E10.

### Intermediate E11: 4-(4-(2-chloro-3-(6-methoxy-S-((7-oxo-2,6-diazaspiro[3,4]octan-2-yl)methyl)pyridin-2-yl)phenyl)-1H-indol-1-yl)-2,6-dimethoxybenzaldehyde

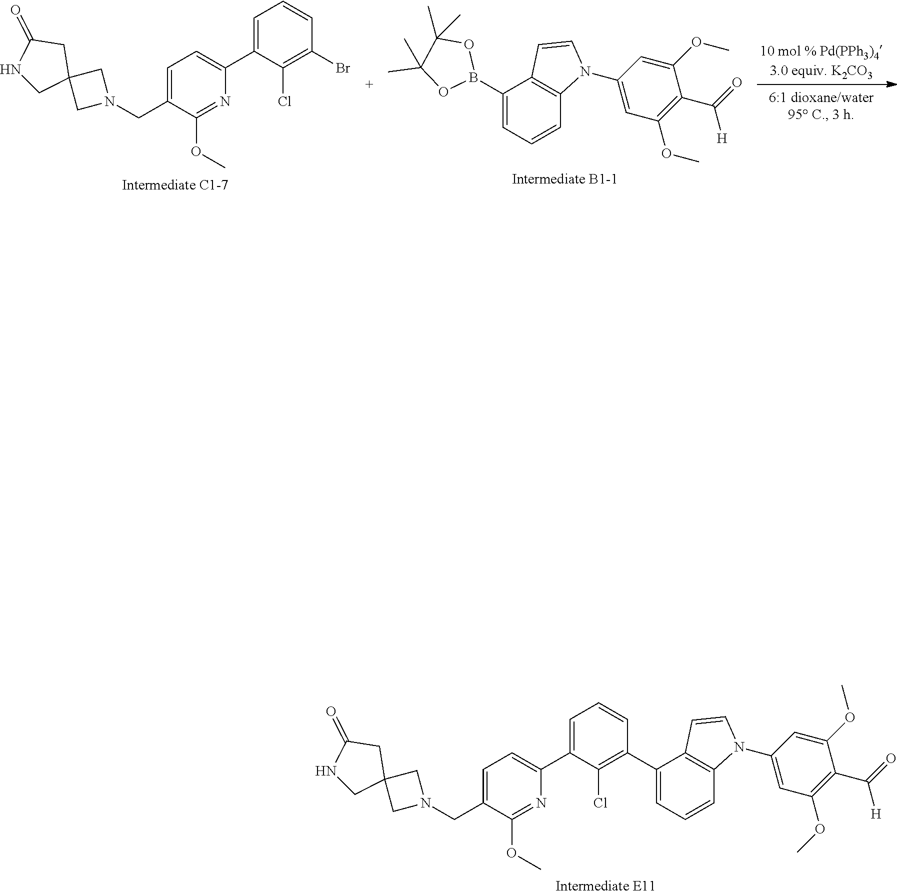 [SMILES: COc1nc(-c2cccc(Br)c2Cl)ccc1CN1CC2(CNC(=O)C2)C1 ; COc1cc(-n2ccc3c(B4OC(C)(C)C(C)(C)O4)cccc32)cc(OC)c1C=O ; COc1cc(-n2ccc3c(-c4cccc(-c5ccc(CN6CC7(CNC(=O)C7)C6)c(OC)n5)c4Cl)cccc32)cc(OC)c1C=O]

A reaction mixture of Intermediate C1-7 and Intermediate B1-1 was processed by using similar procedures as described in Intermediate D1 to yield a crude mixture, which was then purified by column chromatography (silica gel, gradient elution, 5 to 15% MeOH/DCM) to provide Intermediate E11.

### Intermediate E12: 6-(4-(2-chloro-3-(6-methoxy-5-((7-oxo-2,6-diazaspiro[3,4]octan-2-yl)methyl)pyridin-2-yl)phenyl)-1H-indazol-1-yl)-4-methoxynicotinaldehyde

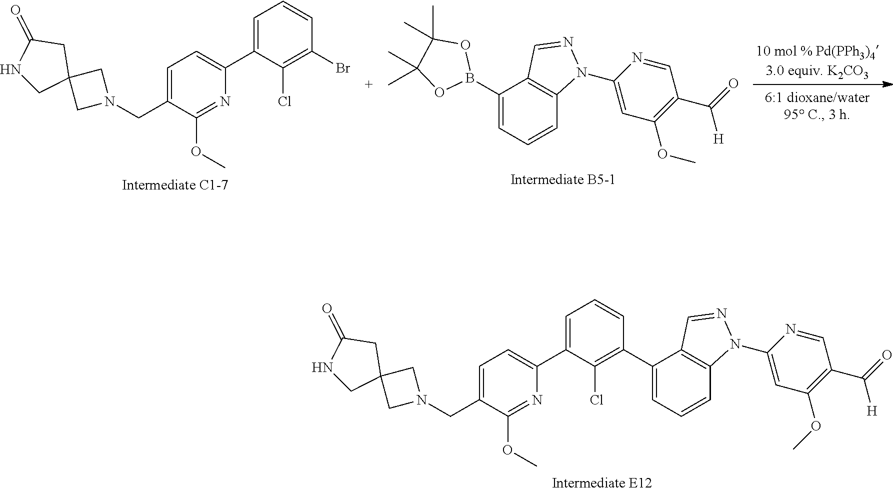 [SMILES: COc1nc(-c2cccc(Br)c2Cl)ccc1CN1CC2(CNC(=O)C2)C1 ; COc1cc(-n2ncc3c(B4OC(C)(C)C(C)(C)O4)cccc32)ncc1C=O ; COc1cc(-n2ncc3c(-c4cccc(-c5ccc(CN6CC7(CNC(=O)C7)C6)c(OC)n5)c4Cl)cccc32)ncc1C=O]

A reaction mixture of Intermediate C1-7 and Intermediate B5-1 was processed by using similar procedures as described in Intermediate D1 to yield a crude mixture, which was then purified by column chromatography (silica gel, gradient elution, 0 to 15% MeOH/DCM) to provide Intermediate E12.

### Intermediate E13: tert-butyl ((6-(2-chloro-3-(1-(5-formyl-4-methoxypyridin-2-yl)-1H-indazol-4-yl)phenyl)-2-methoxypyridin-3-yl)methyl)(cis-3-hydroxycyclobutyl)carbamate

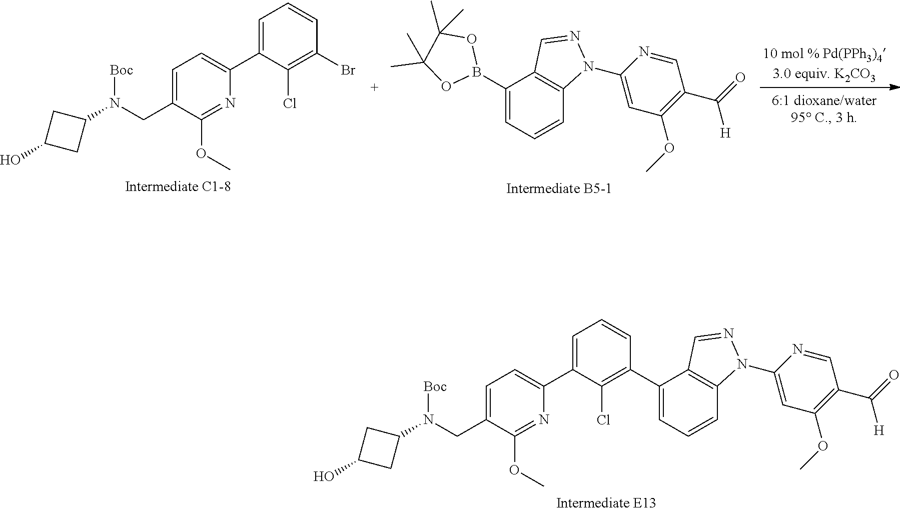

A reaction mixture of Intermediate C1-8 and Intermediate B5-1 was processed by using similar procedures as described in Intermediate D1 to yield a crude mixture, which was then purified by column chromatography (silica gel, gradient elution, 0 to 10% MeOH/DCM) to provide Intermediate E13.

### Intermediate E14: tert-butyl ((6-(2-chloro-3-(1-(5-formyl-4-methoxypyridin-2-yl)-1H-indol-4-yl)phenyl)-2-methoxypyridin-3-yl)methyl)(cis-3-hydroxycyclobutyl)carbamate

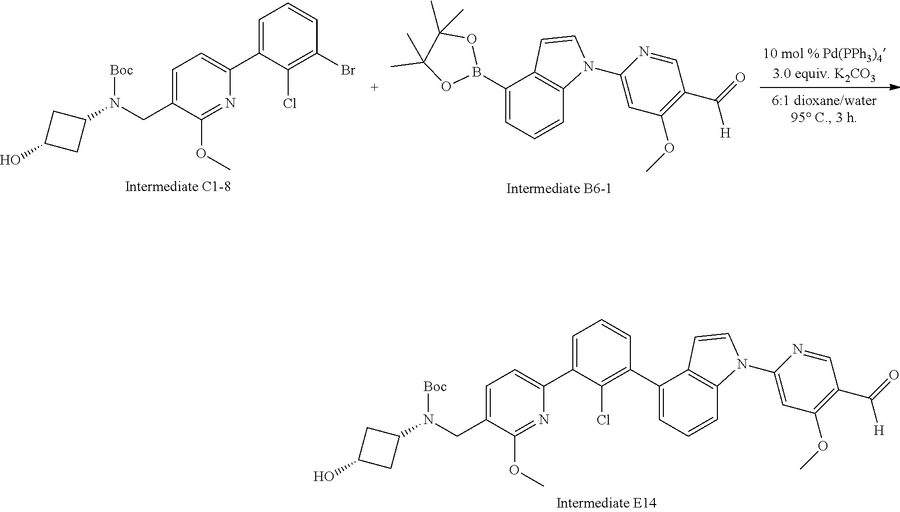

A reaction mixture of Intermediate C1-8 and Intermediate B6-1 was processed by using similar procedures as described in Intermediate D1 to yield a crude mixture, which was then purified by column chromatography (silica gel, gradient elution, 0 to 10% MeOH/DCM) to provide Intermediate E14.

### Intermediate F1 (S)-tert-butyl 4-(4-(2-chloro-3-(5-formyl-6-methoxypyridin-2-yl)phenyl)-1H-indol-1-yl)-2,6-dimethoxybenzyl((5-oxopyrrolidin-2-yl)methyl)carbamate

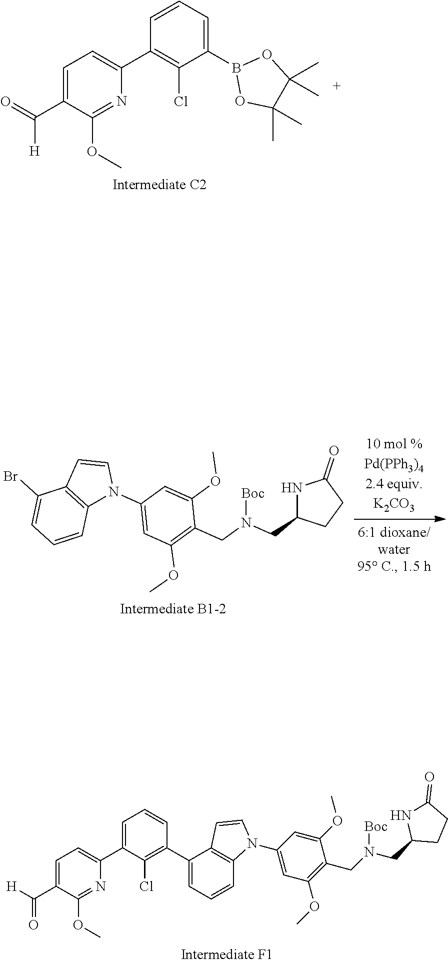 [SMILES: COc1nc(-c2cccc(B3OC(C)(C)C(C)(C)O3)c2Cl)ccc1C=O ; COc1cc(-n2ccc3c(Br)cccc32)cc(OC)c1CN(C[C@@H]1CCC(=O)N1)C(=O)OC(C)(C)C ; COc1cc(-n2ccc3c(-c4cccc(-c5ccc(C=O)c(OC)n5)c4Cl)cccc32)cc(OC)c1CN(C[C@@H]1CCC(=O)N1)C(=O)OC(C)(C)C]

A reaction mixture of Intermediate C2 and Intermediate B1-2 was processed by using similar procedures as described in Intermediate D1 to yield a crude mixture, which was then purified by column chromatography (silica gel, gradient elution, 0 to 3% MeOH/DCM) to provide Intermediate F1.

### Intermediate F2: (S)-tert-butyl 4-(4-(2-chloro-3-(5-formyl-6-methoxypyridin-2-yl)phenyl)-1H-indazol-1-yl)-2,6-dimethoxybenzyl((5-oxopyrrolidin-2-yl)methyl)carbamate

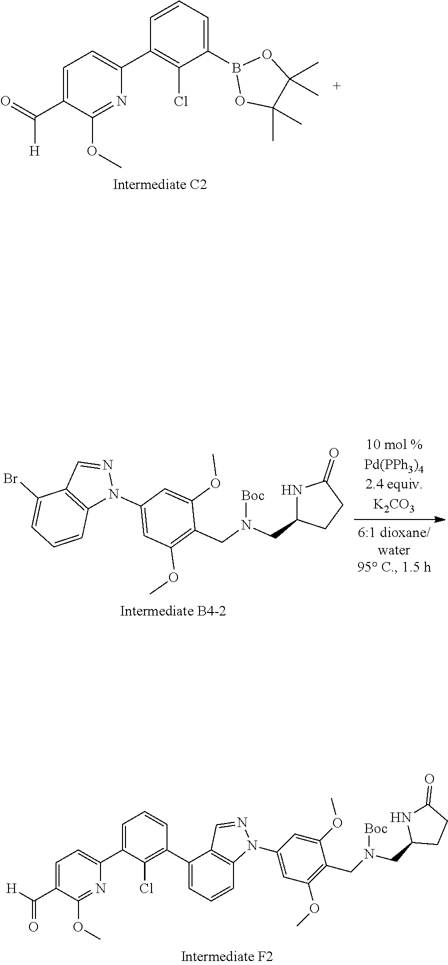 [SMILES: COc1nc(-c2cccc(B3OC(C)(C)C(C)(C)O3)c2Cl)ccc1C=O ; COc1cc(-n2ncc3c(Br)cccc32)cc(OC)c1CN(C[C@@H]1CCC(=O)N1)C(=O)OC(C)(C)C ; COc1cc(-n2ncc3c(-c4cccc(-c5ccc(C=O)c(OC)n5)c4Cl)cccc32)cc(OC)c1CN(C[C@@H]1CCC(=O)N1)C(=O)OC(C)(C)C]

A reaction mixture of Intermediate C2 and Intermediate B4-2 was processed by using similar procedures as described in Intermediate D1 to yield a crude mixture, which was then purified by column chromatography (silica gel, gradient elution, 0 to 3% MeOH/DCM) to provide Intermediate F2.

### Intermediate G: (2-(3-bromo-2-methylphenyl)-7-chlorobenzo[d]oxazol-5-yl) methanol

### Step 1: methyl 3-chloro-4-hydroxy-5-nitrobenzoate

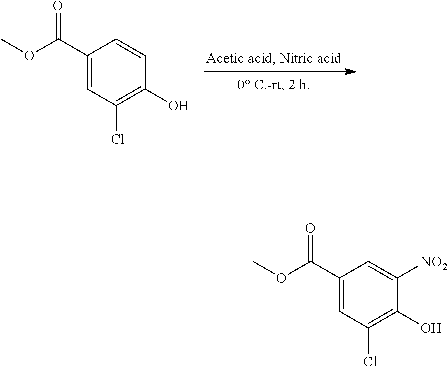

A mixture of acetic acid (20.0 mL, 352 mmol) and nitric acid (4.72 mL, 112 mmol) was added by dropwise to a stirred solution of methyl 3-chloro-4-hydroxybenzoate (10.00 g, 53.6 mmol) in acetic acid (20.0 mL, 352 mmol) at 0° C. The reaction mixture was stirred at room temperature for 2 hours before cold water was added. The mixture was then filtered to obtain the desired product without further purification.

### Step 2: methyl 3-chloro-4-hydroxy-5-nitrobenzoate

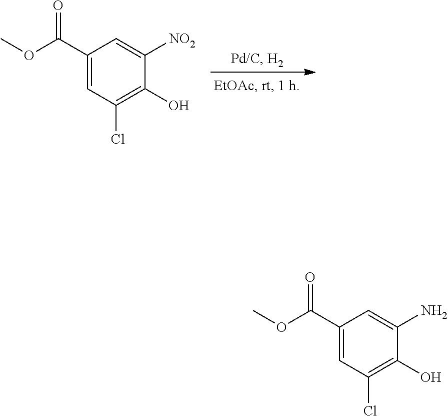

To a solution of methyl 3-chloro-4-hydroxy-5-nitrobenzoate (10.0 g, 43.18 mmol) and palladium on carbon (10 wt %, 2.28 g, 2.16 mmol) in ethyl acetate (100 mL) was stirred at room temperature under hydrogen gas atmosphere for 1 hour. The resulting mixture was filtered and concentrated under reduced pressure to obtain the desired product without further purification.

### Step 3: methyl 2-(3-bromo-2-methylphenyl)-7-chlorobenzo d oxazole-S-carboxylate

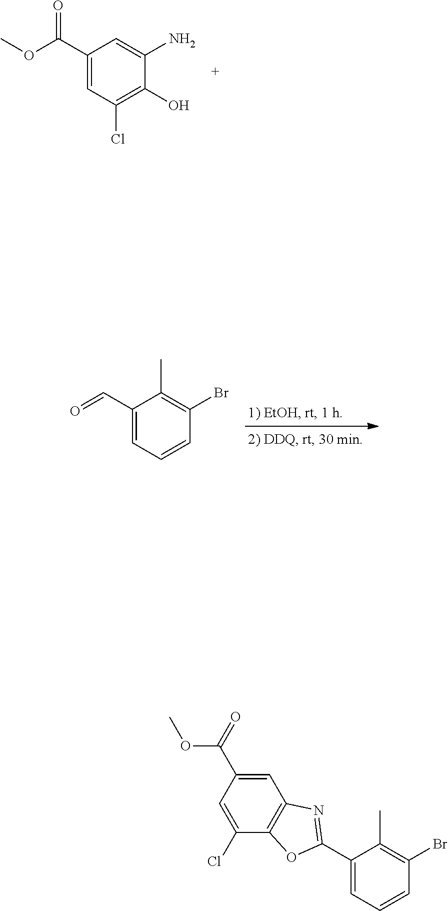

A mixture of methyl 3-chloro-4-hydroxy-5-nitrobenzoate (1.04 g, 5.16 mmol) and 3-bromo-2-methylbenzaldehyde (0.98 g, 4.92 mmol) in ethanol (25 mL) was stirred at room temperature for 1 hour. The mixture was concentrated. The residue was redissolved in dichloromethane (25 mL) and dichlorodicyanoquinone (1.12 g, 4.92 mmol) was added. The mixture was stirred at room temperature for 30 minutes and concentrated. The residue solid was filtered and was washed with methanol to yield the desired product without further purification.

### Step 4: (2-(3-bromo-2-methylphenyl)-7-chlorobenzo[d]oxazol-5-yl)methanol

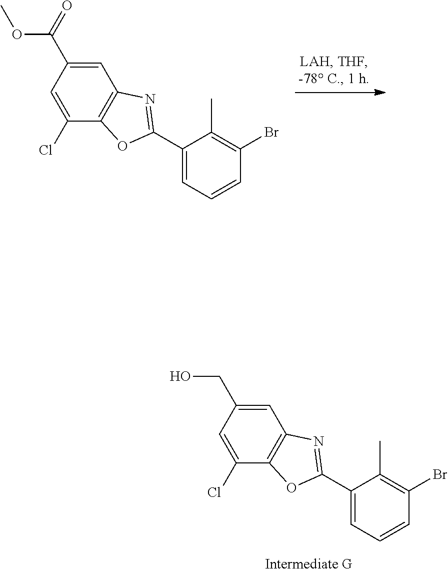 [SMILES: COC(=O)c1cc(Cl)c2oc(-c3cccc(Br)c3C)nc2c1 ; Cc1c(Br)cccc1-c1nc2cc(CO)cc(Cl)c2o1]

Lithium aluminum hydride in THF (14.3 mL, 28.6 mmol) was added to a solution of methyl 2-(3-bromo-2-methylphenyl)-7-chlorobenzo[d]oxazole-5-carboxylate (9.91 g, 26.0 mmol) in THF at −78° C. and stirred for 1 hour before methanol (at 0° C.) was added. The reaction was quenched by a small amount of water and concentrated under reduced pressure. The residue solid was filtered by using EtOAc and concentrated to give the desired product.

### Intermediate G-1: (7-chloro-2-(2-methyl-3-(4,4,5,5-tetramethyl-1,3,2-dioxaborolan-2-yl)phenyl)benzo[d]oxazol-5-yl)methanol

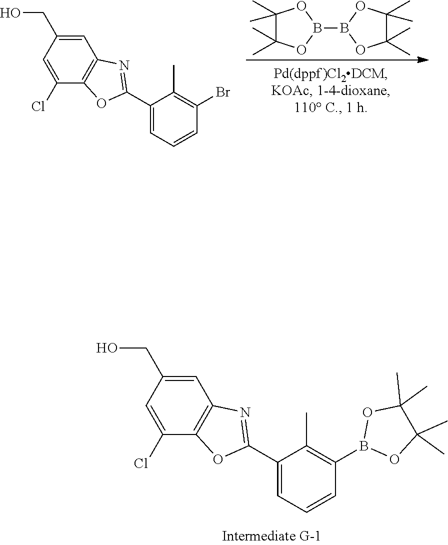

Pd(dppf)Cl₂′CH₂Cl₂ (0.1 g, 0.14 mmol) was added to a mixture of Intermediate G (1.0 g, 2.84 mmol), B₂Pin₂ (0.8 g, 3.40 mmol), and K₂CO₃ (0.8 g, 8.51 mmol) in 1,4-dioxane 50 mL. The resulting suspension was sparged with argon for 10 minutes. After, the reaction was sealed and stirred at 110° C. for 1 hour under argon atmosphere, cooled down and diluted with water. The resulting mixture was extracted 3 times with EtOAc and the organic layer was collected, dried over anhydrous Na₂SO₄, filtered, and evaporated to dryness. The crude mixture was purified by column chromatography (silica gel, 25% EtOAc/Hexanes) to provide Intermediate G-1.

### Intermediate H: 2-(3-(1-(4-formyl-3,5-dimethoxyphenyl)-1H-indazol-4-γ)-2-methylphenyl)-5-(hydroxymethyl)benzo[d]oxazole-7-carbonitrile

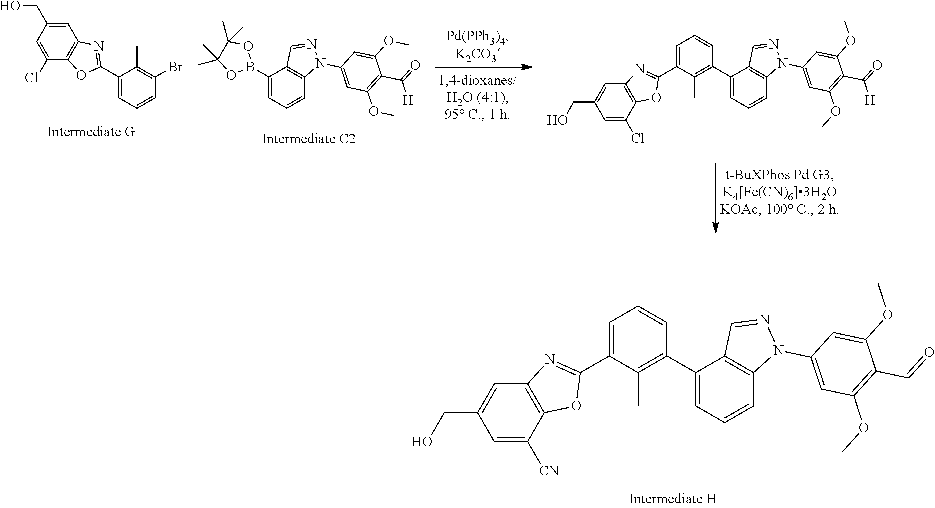

A mixture of Intermediate G (276.0 mg, 0.78 mmol), Intermediate C2 (384.0 mg, 0.94 mmol), Pd(PPh₃)₄ (90.6 mg, 0.08 mmol) and K₂CO₃ (379.0 mg, 2.74 mmol) in a mixture of 1,4-dioxane and H₂O (4:1) 10 mL was purged with argon for 10 mins before stirring at 95° C. for 1.0 hour under argon atmosphere. Then, the mixture was cooled and diluted with H₂O. The resulting mixture was extracted three times with DCM and the organic layer was collected, dried over anhydrous Na₂SO₄, filtered, and evaporated to dryness. The crude mixture was purified by column chromatography (silica gel, gradient elution, 0 to 50% MeOH/DCM) to provide 4-(4-(3-(7-chloro-5-(hydroxymethyl)benzo[d]oxazol-2-yl)-2-methylphenyl)-1H-indazol-1-yl)-2,6-dimethoxybenzaldehyde.

A mixture of 4-(4-(3-(7-chloro-5-(hydroxymethyl)benzo[d]oxazol-2-yl)-2-methylphenyl)-1H-indazol-1-yl)-2,6-dimethoxybenzaldehyde (120 mg, 0.22 mmol), K₄[Fe(CN)₆]·3H₂O (183 mg, 0.43 mmol), tBuXPhos Pd G3 (15.88 mg, 0.02 mmol) and KOAc (74.40 mg, 0.76 mmol) in a mixture of 1-4-dioxane and H₂O (1:1) 10 mL was purged with nitrogen for 10 minutes before stirring at 100° C. for 2 hours under nitrogen atmosphere. Then, the mixture was cooled and diluted with brine. The resulting mixture was extracted three times with EtOAc and the organic layer was collected, dried over anhydrous Na₂SO₄, filtered, and evaporated to dryness. The crude mixture was purified by column chromatography (silica gel, gradient elution, 20 to 40% MeOH/DCM) to provide Intermediate H.

### Intermediate 1: 4-((3-bromo-2-methylphenyl)amino)-2-(difluoromethyl)pyrido[3,2-d]pyrimidine-7-carbaldehyde

### Step 1: 3-amino-5-bromopicolinamide

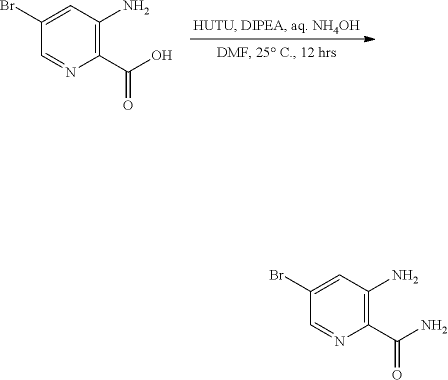 [SMILES: Nc1cc(Br)cnc1C(=O)O ; NC(=O)c1ncc(Br)cc1N]

A mixture of 3-amino-5-bromopicolinic acid (150 g, 691 mmol, 1.00 eq), HATU (289 g, 760 mmol, 1.10 eq), DIPEA (178 g, 1.38 mol, 240 mL, 2.00 eq) in DMF (1.70 L), and aq·NH₄OH (346 g, 2.76 mol, 380 mL, 28.0% purity, 4.00 eq) was stirred at 25° C. for 12 hrs under N₂ atmosphere. H₂O (2.50 L) was added and the reaction mixture extracted with EtOAc (500 mL×6). The combined organic layers were washed with aq·NaCl 2.50 L, dried over Na₂SO₄, filtered and concentrated under reduced pressure, to give the desired product which was used in the next step without further purification. ¹H NMR: (400 MHz, DMSO-d₆) δ 7.91 (br s, 1H), 7.78-7.82 (m, 1H), 7.37-7.44 (m, 2H), 7.04 (br s, 2H).

### Step 2: 5-bromo-3-(2,2-difluoroacetamido)picolinamide

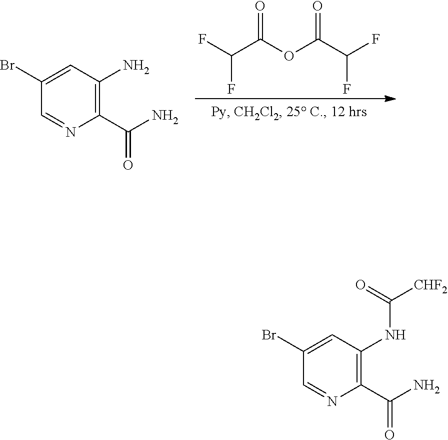 [SMILES: NC(=O)c1ncc(Br)cc1N ; O=C(OC(=O)C(F)F)C(F)F ; NC(=O)c1ncc(Br)cc1NC(=O)C(F)F]

2,2-difluoroacetic anhydride (145 g, 833 mmol, 1.50 eq) was added to a solution of 3-amino-5-bromopicolinamide (120 g, 555 mmol, 1.00 eq) and pyridine (79.0 g, 1.67 mol, 3.00 eq) in CH₂Cl₂ (1.30 L) under N₂ atmosphere at 5-10° C. The mixture was stirred at 25° C. for 12 hours under N₂ atmosphere. The reaction mixture was filtered and concentrated under reduced pressure to give the desired product which was used in the next step without further purification. ¹H NMR: (400 MHz, DMSO-d₆) δ 13.29-13.35 (m, 1H), 9.12 (d, J=2.00 Hz, 1H), 8.64 (br s, 1H), 8.56 (d, J=2.00 Hz, 1H), 8.24 (br s, 1H), 6.36-6.75 (m, 1H).

### Step 3: 7-bromo-2-(difluoromethyl)pyrido[3,2-d]pyrimidin-4-ol

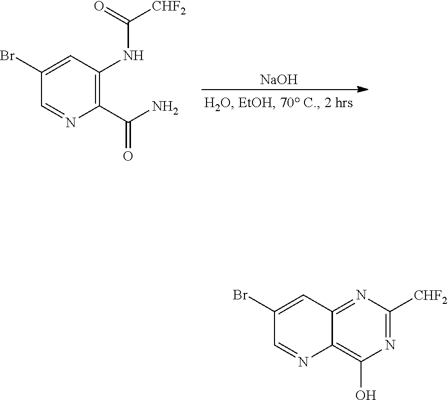 [SMILES: NC(=O)c1ncc(Br)cc1NC(=O)C(F)F ; "NaOH" ; Oc1nc(C(F)F)nc2cc(Br)cnc12]

The mixture of NaOH (17.4 g, 435 mmol, 1.00 eq) and H₂O (100 mL) was slowly added to the mixture of 5-bromo-3-(2,2-difluoroacetamido)picolinamide (128 g, 435 mmol, 1.00 eq) and EtOH (800 mL) H₂O (100 mL) at 70° C. and stirred for 2 hours. The reaction mixture was filtered and concentrated under reduced pressure to give a residue. The desired product was used in the next step without further purification. ¹HNMR: (400 MHz, DMSO-d₆) δ 8.55 (d, J=2.13 Hz, 1H), 8.18 (s, 1H), 6.23-6.57 (m, 1H), 1.05 (t, J=7.00 Hz, 1H).

### Step 4: 2-(difluoromethyl)-7-vinylpyrido[3,2-d]pyrimidin-4-ol

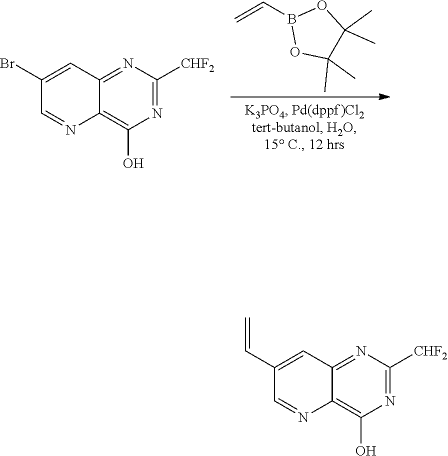 [SMILES: Oc1nc(C(F)F)nc2cc(Br)cnc12 ; C=CB1OC(C)(C)C(C)(C)O1 ; C=Cc1cnc2c(O)nc(C(F)F)nc2c1]

A mixture of 7-bromo-2-(difluoromethyl)pyrido[3,2-d]pyrimidin-4-ol (115 g, 416 mmol, 1.00 eq), 4,4,5,5-tetramethyl-2-vinyl-1,3,2-dioxaborolane (96.2 g, 624 mmol, 106 mL, 1.50 eq), K₃PO₄ (221 g, 1.04 mol, 2.50 eq), Pd(dppf)Cl₂ (15.2 g, 20.8 mmol, 0.05 eq) in tert-butanol (600 mL) and Water (600 mL) was degassed and purged with N₂, and then the mixture was stirred at 15° C. for 12 hrs under N₂ atmosphere. The reaction mixture was filtered and the filtrate was extracted with CH₂Cl₂. The aqueous phase was collected and the pH was adjusted with 0.2 M HCl to around 1, before extraction with CH₂Cl₂ (500 mL×7). The combined organic layers were washed with aq·NaCl (1000 mL), dried over Na₂SO₄, filtered and concentrated under reduced pressure. The residue was stirred with EtOH at 25° C. for 1 hr, filtered to give the desired product. ¹HNMR: (400 MHz, DMSO-d₆) δ 13.18-13.44 (m, 1H). 8.97-9.03 (m, 1H), 8.27 (d, J=1.88 Hz, 1H), 6.93-7.00 (m, 1H). 6.67-6.92 (m, 1H), 6.32 (d, J=17.76 Hz, 1H), 5.65 (d, J=11.26 Hz, 1H).

### Step 5: 4-chloro-2-(difluoromethyl)-7-vinylpyrido[3,2-d]pyrimidine

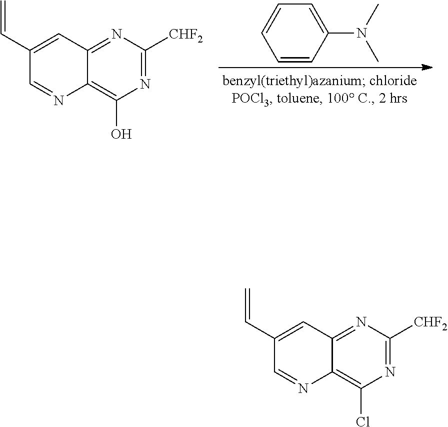

A mixture of 2-(difluoromethyl)-7-vinylpyrido[3,2-d]pyrimidin-4-ol (30.0 g, 134 mmol, 1.00 eq), POCl₃ (41.2 g, 268 mmol, 24.9 mL, 2.00 eq), benzyl(triethyl)aminonium; chloride (61.2 g, 268 mmol, 2.00 eq) and N,N-dimethylaniline (32.5 g, 268 mmol, 34.0 mL, 2.00 eq) in toluene (300 mL) was degassed, purged with N₂, then was stirred at 100° C. for 2 hrs under N₂ atmosphere, and was used for the next reaction.

### Step 6: N-(3-bromo-2-methylphenyl)-2-(difluoromethyl)-7-vinylpyrido[3,2-d]pyrimidin-4-anine

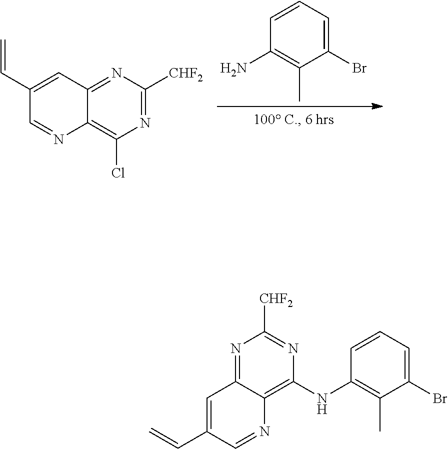

A mixture of 4-chloro-2-(difluoromethyl)-7-vinylpyrido[3,2-d]pyrimidine (30.0 g, 124 mmol, 1.00 eq), 3-bromo-2-methylaniline (30.0 g, 161 mmol, 19.8 mL, 1.30 eq) was degassed, purged with N₂, and then was stirred at 100° C. for 6 hours under N₂ atmosphere. The reaction mixture was concentrated under reduced pressure. The residue was diluted with CH₂Cl₂ 500 mL, then the solution was added dropwise in H₂O 500 mL at under 10° C. (adjust pH to slightly alkaline with NaHCO₃) and extracted with CH₂Cl₂ (500 mL×2). The combined organic layers were washed with aq·NaCl 250 mL, dried over Na₂SO₄, filtered and concentrated under reduced pressure. The residue was purified by flash silica gel chromatography (ISCO®; 330 g SepaFlash® Silica Flash Column, Eluent of 0˜100% Ethyl acetate/Petroleum ether gradient @100 mL/min), stirred in EtOH (50 mL) at 15° C. for 1 hour, and filtered to give the desired product. ¹HNMR: (400 MHz, DMSO-d₆) δ 10.47-10.55 (m, 1H), 9.13-9.20 (m, 1H), 8.35-8.43 (m, 1H), 7.53-7.61 (m, 2H), 7.18-7.27 (m, 1H), 6.96-7.09 (m, 1H), 6.52-6.85 (m, 1H), 6.34-6.43 (m, 1H), 5.64-5.72 (m, 1H), 2.26-2.31 (m, 3H).

### Step 7: (4-((3-bromo-2-methylphenyl)amino)-2-(difluoromethyl)pyrido[3,2-d]pyrimidin-7-yl)methanol

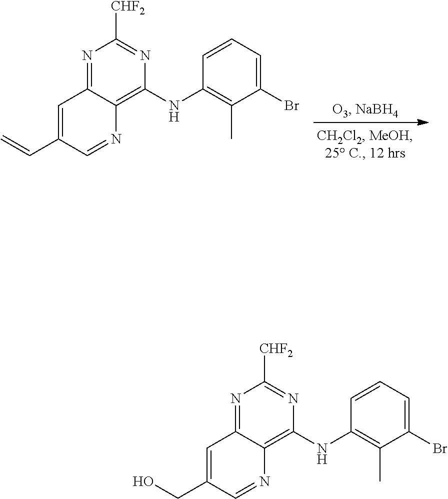 [SMILES: C=Cc1cnc2c(Nc3cccc(Br)c3C)nc(C(F)F)nc2c1 ; Cc1c(Br)cccc1Nc1nc(C(F)F)nc2cc(CO)cnc12]

Ozone was bubbled into a solution of N-(3-bromo-2-methylphenyl)-2-(difluoromethyl)-7-vinylpyrido[3,2-d]pyrimidin-4-amine (17.0 g, 43.4 mmol, 1.00 eq) in MeOH (500 mL) and CH₂Cl₂ (500 mL) 15 minutes. After excess 03 was purged by N₂, NaBH₄ (3.29 g, 86.9 mmol, 2.00 eq) was added at 0° C., then at 20° C. for 4 hrs. The reaction mixture was quenched by addition of H₂O 200 mL at 5° C., and extracted with EtOAc (500 mL 2). The combined organic layers were washed with aq·NaCl 150 mL, dried over Na₂SO₄, filtered and concentrated under reduced pressure. The residue was purified by flash silica gel chromatography (ISCO®; 180 g SepaFlash® Silica Flash Column, Eluent of 0˜100% Ethyl acetate/Petroleum ether gradient @ 100 mL/min) providing the desired product as a white solid.

### Step 8: 4-((3-bromo-2-methylphenyl)amino)-2-(difluoromethyl)pyrido[3,2-d]pyrimidine-7-carbaldehyde

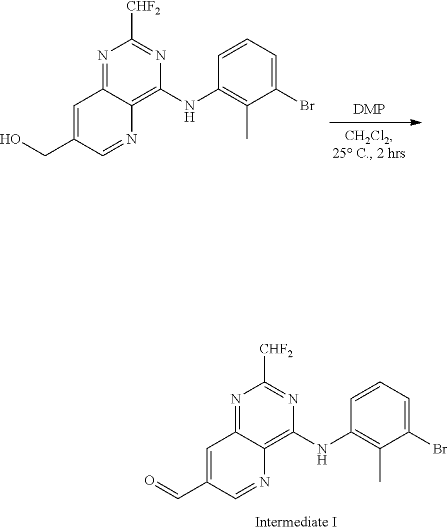

A mixture of (4-((3-bromo-2-methylphenyl)amino)-2-(difluoromethyl)pyrido[3,2-d]pyrimidin-7-yl)methanol (15.0 g, 37.9 mmol, 1.00 eq), DMP (19.3 g, 45.5 mmol, 14.1 mL, 1.20 eq) in CH₂Cl₂ (250 mL) was degassed, purged with N₂, and then stirred at 25° C. for 2 hours under N₂ atmosphere. The reaction solution was added to aq. NaHCO₃, and extracted with CH₂Cl₂ (250 mL×2). The combined organic layers were washed with aq. NaCl 50 mL, dried over Na₂SO₄ filtered and concentrated under reduced pressure. The residue was purified by flash silica gel chromatography (ISCO®; 80 g SepaFlash® Silica Flash Column, Eluent of 0-100% Ethyl acetate/Petroleum ether gradient@100 mL/min) to provide Intermediate 1 as a yellow solid. ¹HNMR: (400 MHz, DMSO-d₆) δ 10.77-10.84 (m, 1H), 10.31-10.37 (m, 1H), 9.31-9.39 (m, 1H), 8.75-8.82 (m, 1H), 7.56-7.63 (m, 1H), 7.50-7.54 (m, 1H), 7.19-7.28 (m, 1H), 6.58-6.90 (m, 1H), 2.27-2.33 (m, 3H).

### Intermediate J: (R)-1-((4-((3-bromo-2-methylphenyl)amino)-2-(difluoromethyl)pyrido[3,2-d]pyrimidin-7-yl)methyl)pyrrolidin-3-ol

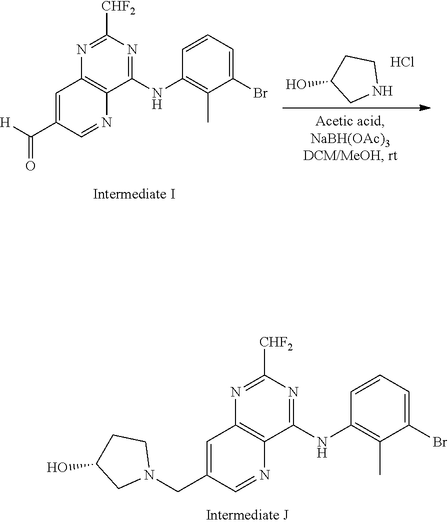 [SMILES: Cc1c(Br)cccc1Nc1nc(C(F)F)nc2cc(C=O)cnc12 ; O[C@@H]1CCNC1 ; Cl ; Cc1c(Br)cccc1Nc1nc(C(F)F)nc2cc(CN3CC[C@@H](O)C3)cnc12]

NEt₃ was added to a solution of (R)-pyrrolidin-3-ol hydrochloride (0.11 g, 0.92 mmol) in 6 mL MeOH/DCM (1:1) until the pH was around 7. Then, the mixture was transferred to a mixture of Intermediate 1 (0.15 g, 0.46 mmol) in 6 mL MeOH/DCM (1:1) at room temperature. The resulting mixture was acidified with acetic acid (pH around 4), stirred for 1 hour, before sodium triacetoxyborohydride (0.49 g, 2.30 mmol) was added. The reaction was stirred at room temperature for an additional 2.5 hours, quenched with a small amount of MeOH and evaporated to dryness. The crude mixture was purified by column chromatography (silica gel, gradient elution, 0 to 20% MeOH/DCM) to provide Intermediate J.

### Intermediate K: (R)-4-(4-(3-((2-(difluoromethyl)-7-((3-hydroxypyrrolidin-1-yl)methyl)pyrido[3,2-d]pyrimidin-4-yl)amino)-2-methylphenyl)-1H-indazol-1-yl)-2,6-dimethoxybenzaldehyde

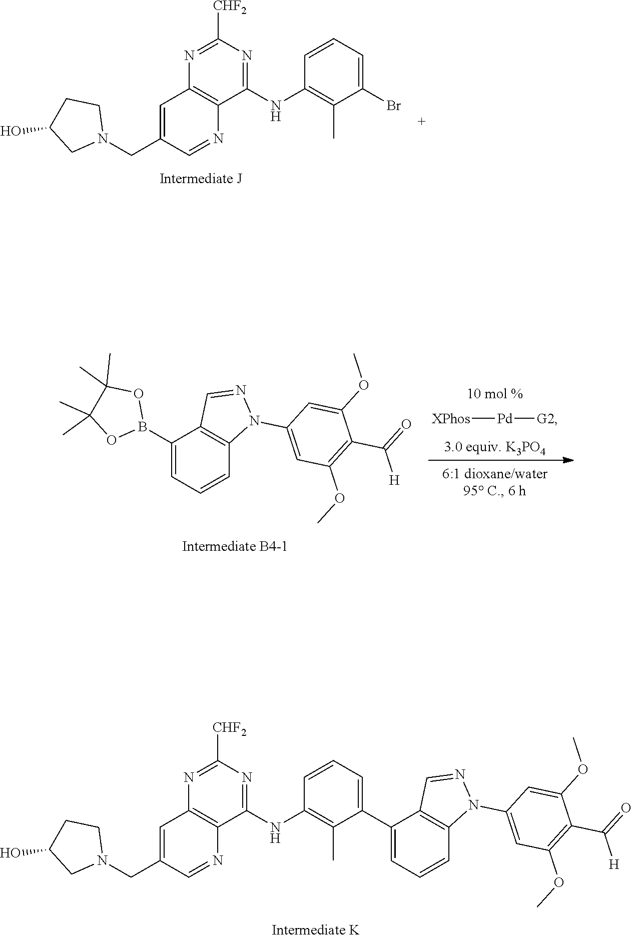

A mixture of Intermediate 1 (0.22 g, 0.48 mmol), Intermediate B4-1 (0.24 g, 0.58 mmol), XPhos-Pd-G2 (0.038 g, 0.048 mmol) and K₃PO₄ (0.31 g, 1.44 mmol) in a mixture of 1,4-dioxane and H₂O (6:1) 14 mL was purged with argon for 10 minutes before it was stirred at 95° C. for 6 h under argon atmosphere. Then, the mixture was cooled and diluted with brine. The resulting mixture was extracted three times with EtOAc and the organic layer was collected, dried over anhydrous Na₂SO₄, filtered, and evaporated to dryness. The crude mixture was purified by column chromatography (silica gel, gradient elution, 0 to 15% MeOH/DCM) to provide Intermediate J.

### Example 1: 2-((4-(4-(2,3-dihydrobenzo[b][1,4]dioxin-6-yl)-1H-indol-1-yl)-2,6-dimethoxybenzyl)amino)-3-hydroxy-2-methylpropanoic acid

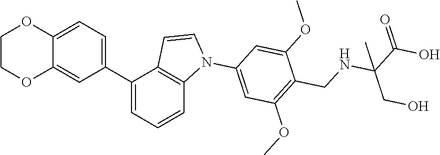 [SMILES: COc1cc(-n2ccc3c(-c4ccc5c(c4)OCCO5)cccc32)cc(OC)c1CNC(C)(CO)C(=O)O]

A mixture of Intermediate A1-1 (59.3 mg, 0.14 mmol) and 2-amino-3-hydroxy-2-methylpropanoic acid (25.5 mg, 0.21 mmol) in 12 mL MeOH/DCM (1:1) was stirred at room temperature and acetic acid was added until the pH was around 4. After the reaction was stirred for 1 hour, sodium cyanoborohydride (31.52 mg, 0.50 mmol) was added and the reaction was stirred at room temperature for overnight. The crude mixture was purified by column chromatography (silica gel, 15% MeOH/DCM). The fractions containing the product were collected and the solvent was removed under reduced pressure to provide the title compound. LCMS Method E, RT=4.30 min, m/z=517.3 [M−H]⁻, exact mass: 518.2053.

### Example 2: (2R,4R)-1-(4-(4-(2,3-dihydrobenzo[b] [1,4]dioxin-6-yl)-1H-indol-1-yl)-2,6-dimethoxybenzyl)-4-hydroxypyrrolidine-2-carboxylic acid

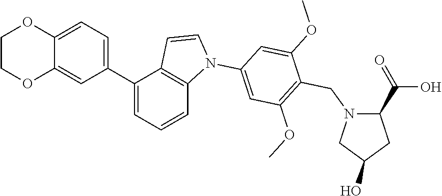 [SMILES: COc1cc(-n2ccc3c(-c4ccc5c(c4)OCCO5)cccc32)cc(OC)c1CN1C[C@H](O)C[C@@H]1C(=O)O]

A mixture of Intermediate A1-1 (81.0 mg, 0.20 mmol) and cis-4-hydroxy-D-proline (38.7 mg, 0.30 mmol) in 16 mL MeOH/DCM (1:1) was stirred at room temperature and acetic acid was added until the pH was around 4. After the reaction was stirred for 1 hour, sodium cyanoborohydride (47.2 mg, 0.75 mmol) was added and the reaction was stirred at room temperature for overnight. The crude mixture was purified by column chromatography (silica gel, 15% MeOH/DCM). The fractions containing the product were collected and the solvent was removed under reduced pressure to provide the title compound. LCMS Method E, RT=3.98 min, m/z=531.3 [M+H]⁺, exact mass: 530.2053.

### Example 3: N-(2-((4-(4-(2,3-dihydrobenzo[b][1,4]dioxin-6-yl)-1H-indol-1-yl)-2,6-dimethoxybenzyl)amino)ethyl)acetamide

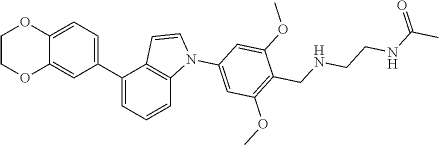 [SMILES: COc1cc(-n2ccc3c(-c4ccc5c(c4)OCCO5)cccc32)cc(OC)c1CNCCNC(C)=O]

A mixture of Intermediate A1-1 (79.7 mg, 0.19 mmol) and N-(2-aminoethyl)acetamide (29.8 mg, 0.29 mmol) in 16 mL MeOH/DCM (1:1) was stirred at room temperature and acetic acid was added until the pH was around 4. After the reaction was stirred for 1 hour, sodium cyanoborohydride (36.5 mg, 0.58 mmol) was added and the reaction was stirred at room temperature for overnight. The crude mixture was purified by column chromatography (silica gel, 15% MeOH/DCM). The fractions containing the product were collected and the solvent was removed under reduced pressure to provide the title compound. LCMS Method E, RT=4.23 min, m/z=502.2 [M+H]⁺, exact mass: 501.2264.

### Example 4: (S)-2-((4-(4-(2,3-dihydrobenzo[b] [1,4]dioxin-6-yl)-1H-indol-1-yl)-2,6-dimethoxybenzyl)amino)-3-hydroxypropanoic acid

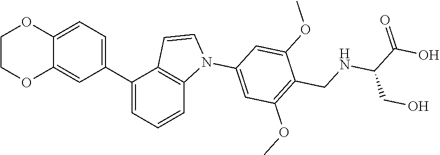 [SMILES: COc1cc(-n2ccc3c(-c4ccc5c(c4)OCCO5)cccc32)cc(OC)c1CN[C@@H](CO)C(=O)O]

A mixture of Intermediate A1-1 (84.0 mg, 0.20 mmol) and L-serine (31.9 mg, 0.30 mmol) in 16 mL MeOH/DCM (1:1) was stirred at room temperature and acetic acid was added until the pH was around 4. After the reaction was stirred for 1 hour, sodium cyanoborohydride (38.1 mg, 0.61 mmol) was added and the reaction was stirred at room temperature for overnight. The reaction mixture was purified by column chromatography (silica gel, 15% MeOH/DCM). The fractions containing the product were collected and the solvent was removed under reduced pressure to provide the title compound. LCMS Method C, RT=4.212 min, m/z=503.2 [M−H]⁻, exact mass: 504.1897.

### Example 5: 2-((2-((3-cyanobenzyl)oxy)-4-(4-(2,3-dihydrobenzo[b][1,4]dioxin-6-yl)-1H-indol-1-yl)benzyl)amino)-3-hydroxy-2-methylpropanoic acid

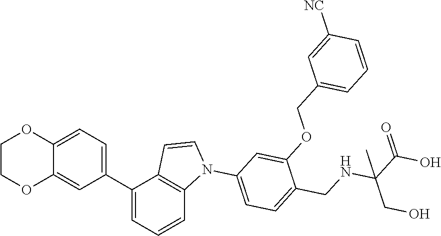 [SMILES: CC(CO)(NCc1ccc(-n2ccc3c(-c4ccc5c(c4)OCCO5)cccc32)cc1OCc1cccc(C#N)c1)C(=O)O]

A mixture of Intermediate A1-2 (50.6 mg, 0.10 mmol) and 2-amino-3-hydroxy-2-methylpropanoic acid (18.0 mg, 0.17 mmol) in 8 mL MeOH/DCM (1:1) was stirred at room temperature and acetic acid was added until the pH was around 4. After the reaction was stirred for 1 hour, sodium cyanoborohydride (30.2 mg, 0.48 mmol) was added and the reaction was stirred at room temperature for overnight. The crude mixture was purified by column chromatography (silica gel, 15% MeOH/DCM). The fractions containing the product were collected and the solvent was removed under reduced pressure to provide the title compound. LCMS Method C, RT=4.367 min, m/z=588.3 [M−H]⁻, exact mass: 589.2213.

### Example 6: 3-((5-(4-(2,3-dihydrobenzo[b][1,4]dioxin-6-yl)-1H-indol-1-yl)-2-(((2-hydroxyethyl)amino)methyl)phenoxy)methyl)benzonitrile

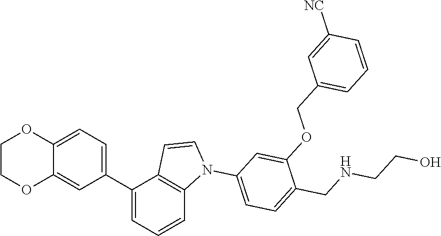 [SMILES: N#Cc1cccc(COc2cc(-n3ccc4c(-c5ccc6c(c5)OCCO6)cccc43)ccc2CNCCO)c1]

A mixture of Intermediate A1-2 (39.8 mg, 0.08 mmol) and 2-aminoethanol (0.01 mL, 0.17 mmol) in 6 mL MeOH/DCM (1:1) was stirred at room temperature and acetic acid was added until the pH was around 4. After the reaction was stirred for 1 hour, sodium cyanoborohydride (27.6 mg, 0.44 mmol) was added and the reaction was stirred at room temperature for overnight. The reaction mixture was purified by column chromatography (silica gel, 15% MeOH/DCM). The fractions containing the product were collected and the solvent was removed under reduced pressure to provide the title compound. LCMS Method C, RT=4.440 min, m/z=532.2 [M+H]⁺, exact mass: 531.2158.

### Example 7: 2-((4-(4-(2,3-dihydrobenzo[b][1,4]dioxin-6-yl)-1H-indole-1-carbonyl)-2,6-dimethoxybenzyl)amino)-3-hydroxy-2-methylpropanoic acid

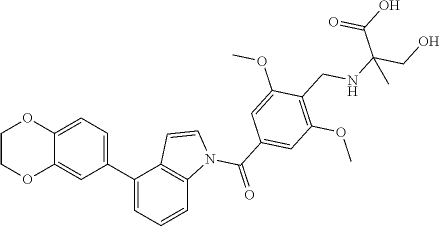 [SMILES: COc1cc(C(=O)n2ccc3c(-c4ccc5c(c4)OCCO5)cccc32)cc(OC)c1CNC(C)(CO)C(=O)O]

Step 1: 4-(4-(2,3-dihydrobenzo[b][1,4]dioxin-6-yl)-1H-indole-1-carbonyl)-2,6-dimethoxybenzaldehyde

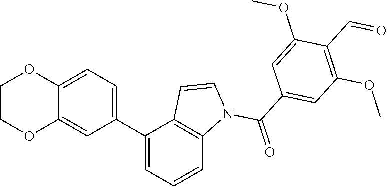 [SMILES: COc1cc(C(=O)n2ccc3c(-c4ccc5c(c4)OCCO5)cccc32)cc(OC)c1C=O]

A mixture of Intermediate A1 (150 mg, 0.60 mmol), 4-bromo-2,6-dimethoxybenzaldehyde (163 mg, 0.67 mmol), Pd(OAc)₂ (4.0 mg, 0.02 mmol), Xantphos (35.5 mg, 0.06 mmol), CsOH·H₂O (897 mg, 5.34 mmol) and CHCl₃ (0.14 mL, 1.75 mmol) in toluene 3 mL was stirred at 80° C. overnight. Then, the mixture was cooled down and diluted with water. The resulting mixture was extracted 3 times with EtOAc and the organic layer was collected, dried over anh. Na₂SO₄, filtered, and evaporated to dryness. The crude mixture was purified by column chromatography (silica gel, gradient elution, 0 to 50% EtOAc/hexanes) to provide 4-(4-(2,3-dihydrobenzo[b][1,4]dioxin-6-yl)-1H-indole-1-carbonyl)-2,6-dimethoxybenzaldehyde.

### Step 2: 2-((4-(4-(2,3-dihydrobenzo[b] [1,4]dioxin-6-yl)-1H-indole-1-carbonyl)-2,6-dimethoxybenzyl)amino)-3-hydroxy-2-methylpropanoic acid

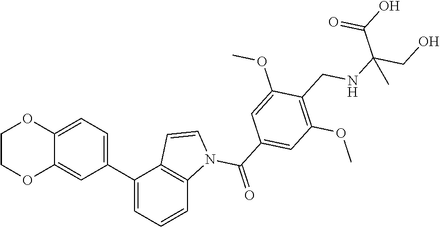 [SMILES: COc1cc(C(=O)n2ccc3c(-c4ccc5c(c4)OCCO5)cccc32)cc(OC)c1CNC(C)(CO)C(=O)O]

A mixture of 4-(4-(2,3-dihydrobenzo[b][1,4]dioxin-6-yl)-1H-indole-1-carbonyl)-2,6-dimethoxybenzaldehyde (33.9 mg, 0.08 mmol) and 2-amino-3-hydroxy-2-methylpropanoic acid (13.0 mg, 0.11 mmol) in 5 mL MeOH/DCM (1:1) was stirred at room temperature and acetic acid was added until the pH was around 4. After the reaction was stirred for 1 hour, sodium cyanoborohydride (21.0 mg, 0.33 mmol) was added and the reaction was stirred at room temperature for overnight. The crude mixture was purified by column chromatography (silica gel, 15% MeOH/DCM). The fractions containing the product were collected and the solvent was removed under reduced pressure to provide the title compound. LCMS Method C, RT=4.430 min, m/z=547.3 [M+H]⁺, exact mass: 546.2002.

### Example 8: 2-(((6-(4-(2,3-dihydrobenzo[b][1,4]dioxin-6-yl)-1H-indol-1-yl)pyridin-3-yl)methyl)amino)ethanol

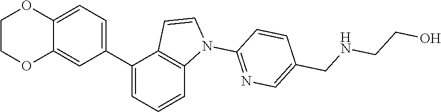 [SMILES: OCCNCc1ccc(-n2ccc3c(-c4ccc5c(c4)OCCO5)cccc32)nc1]

A mixture of Intermediate A1-3 (11.6 mg, 0.03 mmol) and 2-aminoethanol (4 μl, 0.07 mmol) in 2 mL MeOH/DCM (1:1) was stirred at room temperature and acetic acid was added until the pH was around 4. After the reaction was stirred for 1 hour, sodium cyanoborohydride (10.0 mg, 0.15 mmol) was added and the reaction was stirred at room temperature for overnight. The reaction mixture was purified by column chromatography (silica gel, 15% MeOH/DCM). The fractions containing the product were collected and the solvent was removed under reduced pressure to provide the title compound. LCMS Method C, RT=4.229 min, m/z=402.2 [M+H]⁺, LCMS Method D, RT=3.859 min, m/z=402.1781 [M+H]⁺, exact mass: 401.1739.

### Example 9: (S)-2-(((6-(4-(2,3-dihydrobenzo[b] [1,4]dioxin-6-yl)-1H-indol-1-yl)pyridin-3-yl)methyl)amino)-3-hydroxypropanoic acid

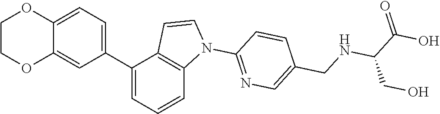 [SMILES: O=C(O)[C@H](CO)NCc1ccc(-n2ccc3c(-c4ccc5c(c4)OCCO5)cccc32)nc1]

A mixture of Intermediate A1-3 (11.6 mg, 0.03 mmol) and L-serine (5.6 mg, 0.05 mmol) in 2 mL MeOH/DCM (1:1) was stirred at room temperature and acetic acid was added until the pH was around 4. After the reaction was stirred for 1 hour, sodium cyanoborohydride (9.6 mg, 0.14 mmol) was added and the reaction was stirred at room temperature for overnight. The reaction mixture was purified by column chromatography (silica gel, 15% MeOH/DCM). The fractions containing the product were collected and the solvent was removed under reduced pressure to provide the title compound. LCMS Method C, RT=4.162 min, m/z=444.2 [M−H]⁻, exact mass: 445.1638.

### Example 10: (S)-2-(((6-(4-(2,3-dihydrobenzo[b][1,4]dioxin-6-yl)-1H-indol-1-yl)-2-methoxypyridin-3-yl)methyl)amino)-3-hydroxypropanoic acid

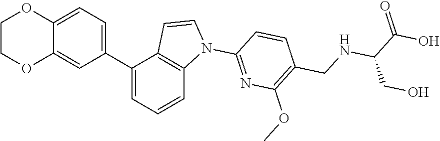 [SMILES: COc1nc(-n2ccc3c(-c4ccc5c(c4)OCCO5)cccc32)ccc1CN[C@@H](CO)C(=O)O]

A mixture of Intermediate A1-4 (50 mg, 0.13 mmol) and L-serine (19.8 mg, 0.19 mmol) in 4 mL MeOH/DCM (1:1) was stirred at room temperature and acetic acid was added until the pH was around 4. After the reaction was stirred for 1 hour, sodium cyanoborohydride (44.2 mg, 0.64 mmol) was added and the reaction was stirred at room temperature for an additional 3 hours. The crude mixture was purified by column chromatography (silica gel, 15% MeOH/DCM). The fractions containing the product were collected and the solvent was removed under reduced pressure to provide the title compound. LCMS Method C, RT=4.283 min, m/z=474.2 [M−H]⁻, LCMS Method D, RT=4.134 min, m/z=476.1174 [M+H]⁺, exact mass: 475.1743.

### Example 11: (4-(4-(2,3-dihydrobenzo[b][1,4]dioxin-6-yl)indolin-1-yl)-2,6-dimethoxybenzyl)-L-serine

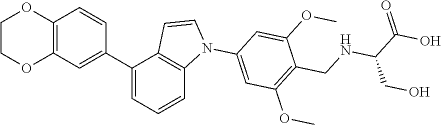 [SMILES: COc1cc(-n2ccc3c(-c4ccc5c(c4)OCCO5)cccc32)cc(OC)c1CN[C@@H](CO)C(=O)O]

A mixture of Intermediate A2-1 (100 mg, 0.24 mmol) and L-serine (29.42 mg, 0.28 mmol) in 6 mL MeOH/DCM (1:1) was stirred at room temperature and acetic acid was added until the pH was around 4. After the reaction was stirred for 1 hour, sodium cyanoborohydride (75.40 mg, 1.20 mmol) was added and the reaction was stirred at room temperature for an additional 1 hour. The crude mixture was purified by column chromatography (silica gel, 10% MeOH/DCM). The fractions containing the product were collected and the solvent was removed under reduced pressure, resulting in the title compound as product. LCMS Method C, RT=4.352 min, m/z=505.3 [M−H]⁻, LCMS Method D, RT=4.263 min, m/z=507.2059 [M+H]⁺, exact mass: 506.2053.

### Example 12: 1-((6-(4-(2,3-dihydrobenzo[b][1,4]dioxin-6-yl)-1H-indol-1-yl)-2-methoxypyridin-3-yl)methyl)-3-methylazetidin-3-ol

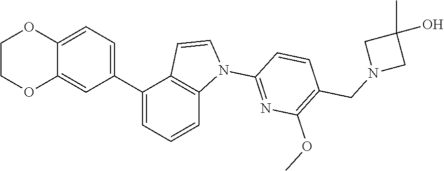 [SMILES: COc1nc(-n2ccc3c(-c4ccc5c(c4)OCCO5)cccc32)ccc1CN1CC(C)(O)C1]

A mixture of Intermediate A1-4 (51.8 mg, 0.13 mmol) and 3-methylazetidin-3-ol hydrochloride (21.3 mg, 0.17 mmol) in 4 mL MeOH/DCM (1:1) was stirred at room temperature and acetic acid was added until the pH was around 4. After the reaction was stirred for 1 hour, sodium cyanoborohydride (37.3 mg, 0.54 mmol) was added and the reaction was stirred at room temperature for overnight. The reaction mixture was purified by column chromatography (silica gel, 15% MeOH/DCM). The fractions containing the product were collected and the solvent was removed under reduced pressure to provide the title compound. LCMS Method C, RT=4.474 min, m/z=458.2 [M+H]⁺, LCMS Method D, RT=4.198 min, m/z=458.2038 [M+H]⁺, exact mass: 457.2002.

### Example 13: (S)-5-(((4-(4-(2,3-dihydrobenzo[b][1,4]dioxin-6-yl)indolin-1-yl)-2,6 dimethoxybenzyl)amino)methyl) pyrrolidin-2-one

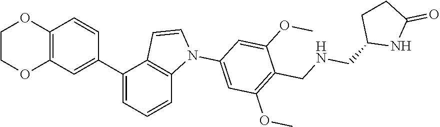 [SMILES: COc1cc(-n2ccc3c(-c4ccc5c(c4)OCCO5)cccc32)cc(OC)c1CNC[C@@H]1CCC(=O)N1]

A mixture of Intermediate A2-1 (100 mg, 0.24 mmol) and (S)-5-(aminomethyl)pyrrolidin-2-one (31.96 mg, 0.28 mmol) in 6 mL MeOH/DCM (1:1) was stirred at room temperature and acetic acid was added until the pH was around 4. After the reaction was stirred for 1 hour, sodium cyanoborohydride (75.40 mg, 1.20 mmol) was added and the reaction was stirred at room temperature for an additional 1 hour. The crude mixture was purified by column chromatography (silica gel, 10% MeOH/DCM). The fractions containing the product were collected and the solvent was removed under reduced pressure, resulting in the title compound as product. LCMS Method C, RT=4.403 min, m/z=538.3 [M+Na]⁺, LCMS Method D, RT=4.192 min, m/z=516.2420 [M+H]⁺, exact mass: 515.2420.

### Example 14: 4-(2,3-dihydrobenzo[b][1,4]dioxin-6-yl)-1-(3,5-dimethoxy-4-(5-methyl-4,5-dihydro-1H-imidazol-2-yl)phenyl)indoline

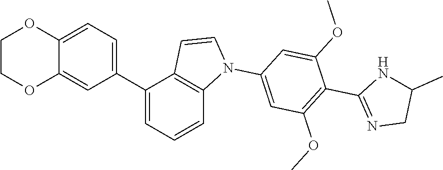 [SMILES: COc1cc(-n2ccc3c(-c4ccc5c(c4)OCCO5)cccc32)cc(OC)c1C1=NCC(C)N1]

A mixture of Intermediate A2-1 (100 mg, 0.24 mmol) and propane-1,2-diamine (20.75 mg, 0.28 mmol) in DCM 4 mL was stirred at room temperature. After 1 hour, NBS (79.84 mg, 0.28 mmol) was added and the reaction was stirred at room temperature for overnight. The reaction mixture was purified by column chromatography (silica gel, 10% MeOH/DCM). The fractions containing the product were collected and the solvent was removed under reduced pressure, resulting in the title compound as product. LCMS Method C, RT=5.0210 min, m/z=472.2 [M+H]⁺, LCMS Method D, RT=4.670 min, m/z=472.2212 [M+H]⁺, exact mass: 471.2158.

### Example 15: (S)-5-(((4-(4-(2,3-dihydrobenzo[b][1,4]dioxin-6-yl)-1H-indol-1-yl)-2,6-dimethoxybenzyl)amino)methoxy)pyrrolidin-2-one

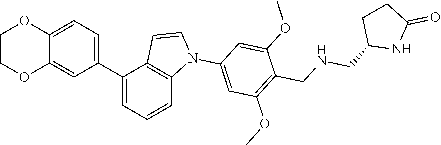 [SMILES: COc1cc(-n2ccc3c(-c4ccc5c(c4)OCCO5)cccc32)cc(OC)c1CNC[C@@H]1CCC(=O)N1]

A mixture of Intermediate A1-1 (85.6 mg, 0.21 mmol) and (S)-5-(aminomethyl)pyrrolidin-2-one (34.0 mg, 0.30 mmol) in 5 mL MeOH/DCM (1:1) was stirred at room temperature and acetic acid was added until the pH was around 4. After the reaction was stirred for 1 hour, sodium cyanoborohydride (63.0 mg, 0.92 mmol) was added and the reaction was stirred at room temperature for overnight. The reaction mixture was purified by column chromatography (silica gel, 15% MeOH/DCM). The fractions containing the product were collected and the solvent was removed under reduced pressure to provide the title compound. LCMS Method C, RT=4.414 min, m/z=536.3 [M+Na]⁺, LCMS Method D, RT=4.181 min, m/z=514.2332 [M+H]⁺, exact mass: 513.2264.

### Example 16: 4-((4-(4-(2,3-dihydrobenzo[b][1,4]dioxin-6-yl)-1H-indol-1-yl)-2,6-dimethoxybenzyl)amino)pyrrolidin-2-one

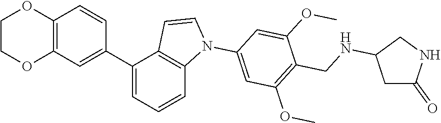 [SMILES: COc1cc(-n2ccc3c(-c4ccc5c(c4)OCCO5)cccc32)cc(OC)c1CNC1CNC(=O)C1]

A mixture of Intermediate A1-1 (100 mg, 0.24 mmol) and 4-aminopyrrolidin-2-one (28.03 mg, 0.28 mmol) in 6 mL MeOH/DCM (1:1) was stirred at room temperature and acetic acid was added until the pH was around 4. After the reaction was stirred for 1 hour, sodium cyanoborohydride (75.40 mg, 1.20 mmol) was added and the reaction was stirred at room temperature for an additional 1 hour. The crude mixture was purified by column chromatography (silica gel, 10% MeOH/DCM). The fractions containing the product were collected and the solvent was removed under reduced pressure, resulting in the title compound as product. LCMS Method C, RT=4.300 min, m/z=522.2 [M+Na]⁺, exact mass: 499.2107.

### Example 17: (S)-5-(((4-(4-(2,3-dihydrobenzo[b] [1,4]dioxin-6-yl)-1H-indol-1-yl)benzyl)amino)methyl)pyrrolidin-2-one

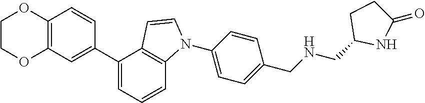 [SMILES: O=C1CC[C@@H](CNCc2ccc(-n3ccc4c(-c5ccc6c(c5)OCCO6)cccc43)cc2)N1]

A mixture of Intermediate A1 (138.20 mg, 0.55 mmol), 4-bromobenzaldehyde (170.21 mg, 0.92 mmol), Pd₂(dba)₃ (18.31 mg, 0.02 mmol), Xantphos (40.50 mg, 0.07 mmol) and Cs₂CO₃ (377.95 mg, 1.16 mmol) in toluene 12 mL was sparged with argon for 10 min before it was refluxed under argon atmosphere for overnight. Then, the mixture was cooled down and diluted with water. The resulting mixture was extracted 3 times with EtOAc and the organic layer was collected, dried over anhydrous Na₂SO₄, filtered, and evaporated to dryness. The crude mixture was purified by column chromatography (silica gel, gradient elution, 0 to 60% EtOAc/hexanes) to provide 4-(4-(2,3-dihydrobenzo[b][1,4]dioxin-6-yl)-1H-indol-1-yl)benzaldehyde.

A mixture of 4-(4-(2,3-dihydrobenzo[b][1,4]dioxin-6-yl)-1H-indol-1-yl)benzaldehyde (85 mg, 0.24 mmol) and (S)-5-(aminomethyl)pyrrolidin-2-one (31.96 mg, 0.28 mmol) in 6 mL MeOH/DCM (1:1) was stirred at room temperature and acetic acid was added until the pH was around 4. After the reaction was stirred for 1 hour, sodium cyanoborohydride (75.40 mg, 1.20 mmol) was added and the reaction was stirred at room temperature for an additional 1 hour. The crude mixture was purified by column chromatography (silica gel, 10% MeOH/DCM). The fractions containing the product were collected and the solvent was removed under reduced pressure, resulting in the title compound. LCMS Method C, RT=4.200 min, m/z=454.2 [M+H]⁺, LCMS Method D, RT=3.959 min, m/z=454.2103 [M+H]⁺, exact mass: 453.2052.

### Example 18: (S)-1-(4-(4-(2,3-dihydrobenzo[b][1,4]dioxin-6-yl)-1H-indol-1-yl)-2,6-dimethoxybenzyl)pyrrolidin-3-ol

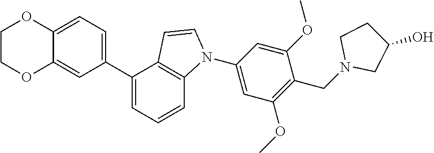 [SMILES: COc1cc(-n2ccc3c(-c4ccc5c(c4)OCCO5)cccc32)cc(OC)c1CN1CC[C@H](O)C1]

A mixture of Intermediate A1-1 (49.0 mg, 0.12 mmol) and (S)-pyrrolidin-3-ol hydrochloride (19.5 mg, 0.16 mmol) in 2 mL MeOH/DCM (1:1) was stirred at room temperature and acetic acid was added until the pH was around 4. After the reaction was stirred for 1 hour, sodium cyanoborohydride (37.5 mg, 0.54 mmol) was added and the reaction was stirred at room temperature for overnight. The reaction mixture was purified by column chromatography (silica gel, 15% MeOH/DCM). The fractions containing the product were collected and the solvent was removed under reduced pressure to provide the title compound. LCMS Method C, RT=4.433 min, m/z=487.2 [M+H]⁺, LCMS Method D, RT=4.287 min, m/z=487.2190 [M+H]⁺, exact mass: 486.2155.

### Example 19: (S)-5-(((3-(4-(2,3-dihydrobenzo[b][1,4]dioxin-6-yl)-1H-indol-1-yl)-5-methoxybenzyl)amino)methyl) pyrrolidin-2-one

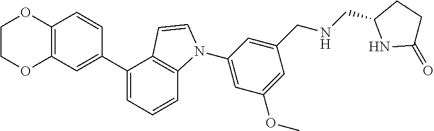 [SMILES: COc1cc(CNC[C@@H]2CCC(=O)N2)cc(-n2ccc3c(-c4ccc5c(c4)OCCO5)cccc32)c1]

This compound was prepared and purified using similar procedures as described in Example 17 replacing 4-bromobenzaldehyde to 3-bromo-5-methoxybenzaldehyde in order to obtain the title compound. LCMS Method C, RT=4.294 min, m/z=484.3 [M+H]⁺, LCMS Method D, RT=4.071 min, m/z=484.2192 [M+H]⁺, exact mass: 483.2158.

### Example 20: (S)-5-((((5-(4-(2,3-dihydrobenzo[b][1,4]dioxin-6-yl)-1H-indol-1-yl)pyridin-3-yl)methyl)amino) methyl)pyrrolidin-2-one

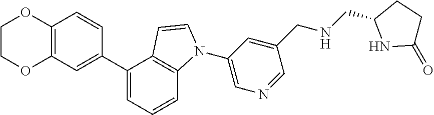 [SMILES: O=C1CC[C@@H](CNCc2cncc(-n3ccc4c(-c5ccc6c(c5)OCCO6)cccc43)c2)N1]

This compound was prepared and purified using similar procedures as described in Example 17 replacing 4-bromobenzaldehyde to 5-bromonicotinaldehyde in order to obtain the title compound. LCMS Method C, RT=4.163 min, m/z=455.2 [M+H]⁺, LCMS Method D, RT=3.710 min, m/z=455.2037 [M+H]⁺, exact mass: 454.2005.

### Example 21: 1-(6-((2-(4-(2,3-dihydrobenzo[b][1,4]dioxin-6-yl)-1H-indol-1-yl)-6-methylpyridin-4-yl)methyl)-2,6-diazaspiro[3.3]heptan-2-yl)ethan-1-one

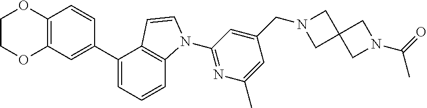 [SMILES: CC(=O)N1CC2(CN(Cc3cc(C)nc(-n4ccc5c(-c6ccc7c(c6)OCCO7)cccc54)c3)C2)C1]

### Step 1: 1-(4-((2,6-diazaspiro[3.3]heptan-2-yl)methyl)-6-methylpyridin-2-yl)-4-(2,3-dihydrobenzo[b][1,4]dioxin-6-yl-1H-indole

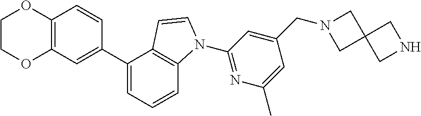 [SMILES: Cc1cc(CN2CC3(CNC3)C2)cc(-n2ccc3c(-c4ccc5c(c4)OCCO5)cccc32)n1]

A mixture of Intermediate A1-5 (80 mg, 0.22 mmol) and tert-butyl 2,6-diazaspiro[3.3]heptane-2-carboxylate (107.2 mg, 0.24 mmol) in MeOH:DCM (5:3, 5 mL) was stirred at room temperature and acetic acid was added until the pH was around 4. After the reaction was stirred for 1 hour, sodium cyanoborohydride (69.12 mg, 1.10 mmol) was added. The reaction was stirred at room temperature for an additional 1 hour, quenched by a small amount of MeOH and evaporated to dryness. Then, the residue was dissolved in dioxane and 4M HCl (1 mL) was added. After the reaction was stirred at room temperature for 30 minutes, the residual solvent was removed under reduced pressure and the residue was washed with dioxane. The crude mixture was purified by column chromatography (silica gel, gradient elution, 0 to 10/MeOH/EtOAc) to provide 1-(4-((2,6-diazaspiro[3.3]heptan-2-yl)methyl)-6-methylpyridin-2-yl)-4-(2,3-dihydrobenzo[b][1,4]dioxin-6-yl)-1H-indole.

### Step 2: 1-(6-((2-(4-(2,3-dihydrobenzo[b][1,4]dioxin-6-yl)-1H-indol-1-yl)-6-methylpyridin-4-yl)methyl)-2,6-diazaspiro[3.3]heptan-2-yl)ethan-1-one

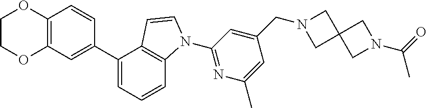 [SMILES: CC(=O)N1CC2(CN(Cc3cc(C)nc(-n4ccc5c(-c6ccc7c(c6)OCCO7)cccc54)c3)C2)C1]

A mixture of 1-(4-((2,6-diazaspiro[3.3]heptan-2-yl)methyl)-6-methylpyridin-2-yl)-4-(2,3-dihydrobenzo[b][1,4]dioxin-6-yl)-1H-indole (24 mg, 0.05 mmol) and acetic anhydride (5.12 μl, 0.05 mmol) in THF (3 mL) was stirred at room temperature for 30 minutes. Then, the resulting mixture was extracted 3 times with EtOAc and the organic layer was collected, dried over anhydrous Na₂SO₄, filtered, and evaporated to dryness. The crude mixture was purified by column chromatography (silica gel, gradient elution, 0 to 5% MeOH/EtOAc) to provide the title compound. LCMS method C, RT=4.335 min, m/z=495.2 [M+H]⁺, LCMS Method D, RT=4.053 min, m/z=495.2352 [M+H]⁺, exact mass. 494.2300.

### Example 22: (S)-5-((((2-(4-(2,3-dihydrobenzo[b][1,4]dioxin-6-yl)-1H-indol-1-yl)-6-methylpyridin-4-yl)methyl)amino)methyl)pyrrolidin-2-one

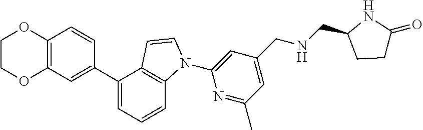 [SMILES: Cc1cc(CNC[C@@H]2CCC(=O)N2)cc(-n2ccc3c(-c4ccc5c(c4)OCCO5)cccc32)n1]

A mixture of Intermediate A1-5 (50 mg, 0.14 mmol) and (S)-5-(aminomethyl)pyrrolidin-2-one (60.8 mg, 0.27 mmol) in MeOH:DCM (5:3, 8 mL) was stirred at room temperature and acetic acid was added until the pH was around 4. After the reaction was stirred for 1 hour, sodium cyanoborohydride (44 mg, 0.70 mmol) was added. The reaction was stirred at room temperature for an additional 1 hour, quenched by a small amount of MeOH and evaporated to dryness. The crude mixture was purified by column chromatography (silica gel, gradient elution, 80% to 100% EtOAc/hexanes) to provide the title compound. LCMS method A, RT=2.844 min, m/z=469.2 [M+H]⁺, 491.2 [M+Na]⁺, LCMS Method D, RT=4.005 min, m/z=469.2201 [M+H]⁺, exact mass: 468.2161.

### Example 23: (S)-5-(((4-(7-(2,3-dihydrobenzo[b][1,4]dioxin-6-yl)-1-methyl-1H-indol-3-yl)-2,6-dimethoxybenzyl)amino)methyl)pyrrolidin-2-one

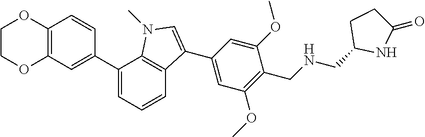 [SMILES: COc1cc(-c2cn(C)c3c(-c4ccc5c(c4)OCCO5)cccc23)cc(OC)c1CNC[C@@H]1CCC(=O)N1]

### Step 1: 7-(2,3-dihydrobenzo[b][1,4]dioxin-6-yl)-1H-indole

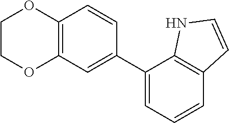 [SMILES: c1cc(-c2ccc3c(c2)OCCO3)c2[nH]ccc2c1]

Pd(PPh₃)₄ (0.27 g, 0.23 mmol) was added to a solution of 7-bromoindole (0.45 g, 2.32 mmol) and 1,4-benzodioxane-6-boronic acid (0.50 g, 2.78 mmol) in 1,4-dioxane 24 mL. A solution of K₂CO₃ (0.77 g, 5.57 mmol) in H₂O 4 mL was added to the reaction mixture before it was sparged with argon for 10 min. After the reaction mixture was stirred at 95° C. for 3 h under argon atmosphere, it was cooled down and diluted with brine. The resulting mixture was extracted 3 times with EtOAc and the organic layer was collected, dried over anhydrous Na₂SO₄, filtered, and evaporated to dryness. The crude mixture was purified by column chromatography (silica gel, gradient elution, 0 to 25% EtOAc/hexanes) to provide 7-(2,3-dihydrobenzo[b][1,4]dioxin-6-yl)-1H-indole.

### Step 2: 7-(2,3-dihydrobenzo[b][1,4]dioxin-6-yl)-3-iodo-1H-indole

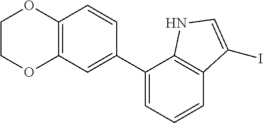 [SMILES: Ic1c[nH]c2c(-c3ccc4c(c3)OCCO4)cccc12]

Fresh KOH (0.35 g, 2.47 mmol) was added into a solution of 7-(2,3-dihydrobenzo[b][1,4]dioxin-6-yl)-1H-indole (0.62 g, 2.47 mmol) in 1 mL of DMF at 0° C. After 30 min, a solution of 12 (0.66 g. 2.59 mmol) in DMF (3M) was added and stirred for 15 min. Then the resulting mixture was extracted 3 times with EtOAc and the organic layer was collected, dried over anhydrous Na₂SO₄, filtered, and evaporated to dryness. The crude mixture was purified by column chromatography (silica gel, gradient elution, 0 to 60% EtOAc/hexanes) to provide 7-(2,3-dihydrobenzo[b][1,4]dioxin-6-yl)-3-iodo-1H-indole.

### Step 3: 7-(2,3-dihydrobenzo[b][1,4]dioxin-6-yl)-3-iodo-1-methyl-1H-indole

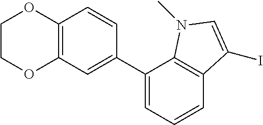 [SMILES: Cn1cc(I)c2cccc(-c3ccc4c(c3)OCCO4)c21]

7-(2,3-dihydrobenzo[b][1,4]dioxin-6-yl)-3-iodo-1H-indole (100 mg, 0.26 mmol) was dissolved in THE (10 mL) before adding NaH (7.00 mg, 0.29 mmol) at 0° C. After the reaction was stirred for 15 minutes, MeI (18 μl, 0.29 mmol) was added and the mixture was stirred at room temperature for 3 hours. The reaction was quenched by adding a small amount of MeOH and evaporated to dryness. The resulting mixture was extracted 3 times with EtOAc and the organic layer was collected, dried over anhydrous Na₂SO₄, filtered, and evaporated to dryness. The crude mixture was purified by column chromatography (silica gel, gradient elution, 20% EtOAc/hexanes) to provide 7-(2,3-dihydrobenzo[b][1,4]dioxin-6-yl)-3-iodo-1-methyl-1H-indole.

### Step 4: 4-(7-(2,3-dihydrobenzo[b][1,4]dioxin-6-yl)-1-methyl-1H-indol-3-yl)-2,6-dimethoxybenzaldehyde

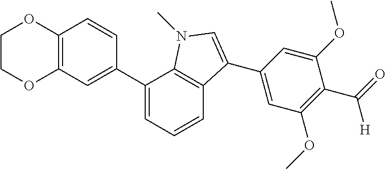 [SMILES: COc1cc(-c2cn(C)c3c(-c4ccc5c(c4)OCCO5)cccc23)cc(OC)c1C=O]

A mixture containing Pd(dppf)₂Cl₂ (24.12 g, 0.03 mmol), 7-(2,3-dihydrobenzo[b][1,4]dioxin-6-yl)-3-iodo-1-methyl-1H-indole (0.13 g, 0.33 mmol), 2,6-dimethoxy-4-(4,4,5,5-tetramethyl-1,3,2-dioxaborolan-2-yl)benzaldehyde (0.15 g, 0.50 mmol, prepared using similar as described for Intermediate B7), and cesium carbonate (0.33 g, 0.99 mmol) in 1,4-dioxane 10 mL was sparged with argon for 10 min. After the reaction mixture was stirred at 95° C. for 3 h under argon atmosphere, it was cooled down and diluted with brine. The resulting mixture was extracted 3 times with EtOAc and the organic layer was collected, dried over anhydrous Na₂SO₄, filtered, and evaporated to dryness. The crude mixture was purified by column chromatography (silica gel, gradient elution, 30 to 50% EtOAc/hexanes) to provide 4-(7-(2,3-dihydrobenzo[b][1,4]dioxin-6-yl)-1-methyl-1H-indol-3-yl)-2,6-dimethoxybenzaldehyde.

### Step 6: (S)-5-(((4-(7-(2,3-dihydrobenzo[b][1,4]dioxin-6-yl)-1-methyl-1H-indol-3-yl)-2,6-dimethoxybenzyl)amino)methyl)pyrrolidin-2-one

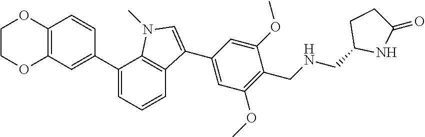 [SMILES: COc1cc(-c2cn(C)c3c(-c4ccc5c(c4)OCCO5)cccc23)cc(OC)c1CNC[C@@H]1CCC(=O)N1]

A mixture of 4-(7-(2,3-dihydrobenzo[b] [1,4]dioxin-6-yl)-1-methyl-JH-indol-3-yl)-2,6-dimethoxybenzaldehyde (60 mg, 0.14 mmol) and (S)-5-(aminomethyl)pyrrolidin-2-one (31.88 mg, 0.28 mmol) in MeOH:DCM (5:3, 14 mL) was stirred at room temperature and acetic acid was added until the pH was around 4. After the reaction was stirred for 1 hour, sodium cyanoborohydride (43.89 mg, 0.70 mmol) was added. The reaction was stirred at room temperature for an additional 1 hour, quenched by a small amount of MeOH and evaporated to dryness. The crude mixture was purified by column chromatography (silica gel, gradient elution, 80 to 100% EtOAc/hexanes) to provide the title compound. LCMS method A, RT=2.933 min, m/z=528.2 [M+H]⁺, 414.2 [M+H−114]⁺, LCMS Method D, RT=4.262 min, m/z=528.2502 [M+H]⁺, exact mass: 527.2420.

### Example 24: (S)-5-(((4-(7-(2,3-dihydrobenzo[b][1,4]dioxin-6-yl)-1H-indol-3-yl)-2,6-dimethoxybenzyl)amino)methyl)pyrrolidin-2-one

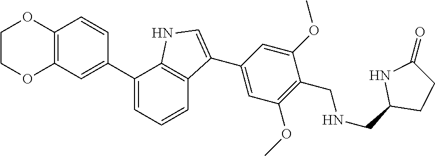 [SMILES: COc1cc(-c2c[nH]c3c(-c4ccc5c(c4)OCCO5)cccc23)cc(OC)c1CNC[C@@H]1CCC(=O)N1]

### Step 1: 4-(7-(2,3-dihydrobenzo[b][1,4]dioxin-6-yl)-1H-indol-3-yl)-2,6-dimethoxybenzaldehyde

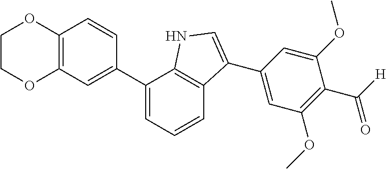 [SMILES: COc1cc(-c2c[nH]c3c(-c4ccc5c(c4)OCCO5)cccc23)cc(OC)c1C=O]

An intermediate 7-(2,3-dihydrobenzo[b][1,4]dioxin-6-yl)-3-iodo-1H-indole was prepared by using the procedures described in Example 23. A mixture of 7-(2,3-dihydrobenzo[b][1,4]dioxin-6-yl)-3-iodo-1H-indole (0.47 g, 1.24 mmol), 4-bromo-2,6-dimethoxybenzaldehyde (1.12 g, 4.59 mmol), Pd(dppf)Cl₂ (0.09 g, 0.13 mmol) and Cs₂CO₃ (1.21 g, 3.72 mmol) in dioxane 15 mL was sparged with argon for 10 min before stirring at 85° C. for 2 h under argon atmosphere. The resulting mixture was cooled down, filtered, and evaporated to dryness. The crude mixture was purified by column chromatography (silica gel, gradient elution, 20 to 60% EtOAc/hexanes) to provide 4-(7-(2,3-dihydrobenzo[b][1,4]dioxin-6-yl)-1H-indol-3-yl)-2,6-dimethoxybenzaldehyde.

### Step 2: (S)-5-(((4-(7-(2,3-dihydrobenzo[b][1,4]dioxin-6-yl)-1H-indol-3-yl)-2,6-dimethoxybenzyl)amino)methyl)pyrrolidin-2-one

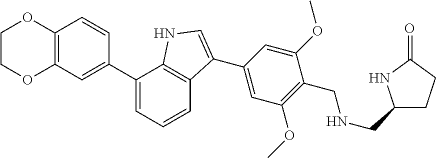 [SMILES: COc1cc(-c2c[nH]c3c(-c4ccc5c(c4)OCCO5)cccc23)cc(OC)c1CNC[C@@H]1CCC(=O)N1]

A mixture of 4-(7-(2,3-dihydrobenzo[b][1,4]dioxin-6-yl)-1H-indol-3-yl)-2,6-dimethoxybenzaldehyde (0.07 g, 0.17 mmol) and (S)-5-(aminomethyl)pyrrolidin-2-one (0.02 g, 0.20 mmol) in 4 mL MeOH/DCM (1:1) was stirred at room temperature and acetic acid was added until the pH was around 4. After the reaction was stirred for 1 hour, sodium cyanoborohydride (0.06 g, 1.00 mmol) was added and the reaction was stirred at room temperature for an additional 1 hour. The crude mixture was purified by column chromatography (silica gel, 10% MeOH/DCM). The fractions containing the product were collected and the solvent was removed under reduced pressure, resulting in the title compound. LCMS Method B, RT=2.868 min, m/z=400.2 [M+H−114]⁺, LCMS Method D, RT=4.091 min, m/z=514.2339 [M+H]⁺, exact mass: 513.2300.

### Example 25: 3-(((3-(4-(2,3-dihydrobenzo[b][1,4]dioxin-6-yl)-1H-indol-1-yl)-1-methyl-1H-pyrrolo[3,2-b]pyridin-6-yl)methyl)amino)-2,2-dimethylpropanoic acid

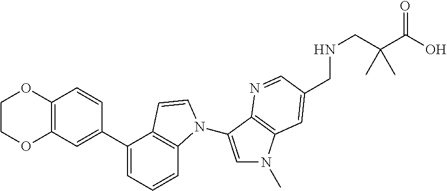 [SMILES: Cn1cc(-n2ccc3c(-c4ccc5c(c4)OCCO5)cccc32)c2ncc(CNCC(C)(C)C(=O)O)cc21]

### Step 1: 1H-pyrrolo[3,2-b]pyridine-6-carbaldehyde

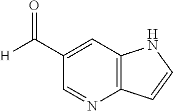 [SMILES: O=Cc1cnc2cc[nH]c2c1]

6-Bromo-1H-pyrrolo[3,2-b]pyridine (0.50 g, 2.54 mmol) was dissolved in dry THF under argon atmosphere then cooled to 10-15° C. Sodium hydride (0.24 g, 10.2 mmol) was added and the reaction was stirred at 10-15° C. for 15 min. After cooling to −70° C., 2M ⁿBuLi (3.20 mL, 6.35 mmol) was added dropwise and the mixture was stirred at −70° C. for 20 min. Dry DMF (1.18 mL, 15.2 mmol) was added into the mixture and additionally stirred for 2 h at −70° C. The reaction was quenched by saturated NH₄C₁, extracted 3 times with EtOAc. The organic layer was collected, dried over anhydrous Na₂SO₄, filtered, and evaporated to provide 1H-pyrrolo[3,2-b]pyridine-6-carbaldehyde.

### Step 2: 3-bromo-1-methyl-1H-pyrrolo[3,2-b]pyridine-6-carbaldehyde

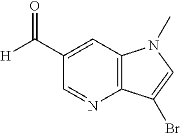

A mixture of 1H-pyrrolo[3,2-b]pyridine-6-carbaldehyde (0.36 g, 2.46 mmol) and N-Bromosuccinimide (0.44 mg, 2.46 mmol) in 24 mL MeOH was stirred at room temperature for overnight. The mixture was extracted 3 times with EtOAc and the organic layer was collected, dried over anhydrous Na₂SO₄, filtered, and evaporated to dryness. The resulting oil was dissolved in DMF and cooled to 0° C. Then sodium hydride (0.08 g, 3.18 mmol) was added and stirred while warm up to room temperature. MeI (0.40 g, 2.80 mmol) was added to the mixture at 0° C. and stirred for 2 h. The reaction was quenched by slowly adding water then extracted 3 times with EtOAc. The organic layer was collected, dried over anhydrous Na₂SO₄, filtered, and evaporated to dryness. The desired product was further used without purification.

### Step 3: 3-(4-(2,3-dihydrobenzo[b][1,4]dioxin-6-yl)-1H-indol-1-yl)-1-methyl-1H-pyrrolo[3,2-b]pyridine-6-carbaldehyde

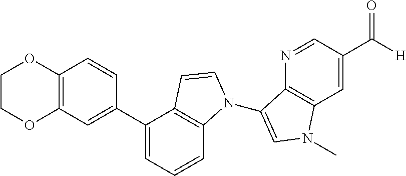 [SMILES: Cn1cc(-n2ccc3c(-c4ccc5c(c4)OCCO5)cccc32)c2ncc(C=O)cc21]

A mixture of Intermediate A1 (0.06 g, 0.23 mmol), 3-bromo-1-methyl-1H-pyrrolo[3,2-b]pyridine-6-carbaldehyde (0.07 g, 0.30 mmol), Pd(dba)₃ (0.01 g, 0.01 mmol), Xantphos (0.01 g, 0.02 mmol) and Cs₂CO₃ (0.26 g, 0.81 mmol) in toluene 15 mL was sparged with argon for 10 min before it was refluxed under argon atmosphere for 16 h. The resulting mixture was cooled down, filtered, and evaporated to dryness. The crude mixture was purified by column chromatography (silica gel, gradient elution, 20 to 80% EtOAc/hexanes).

### Step 4: 3-(((3-(4-(2,3-dihydrobenzo[b][1,4]dioxin-6-yl)-1H-indol-1-yl)-1-methyl-1H-pyrrolo[3,2-b]pyridin-6-yl)methyl)amino)-2,2-dimethylpropanoic acid

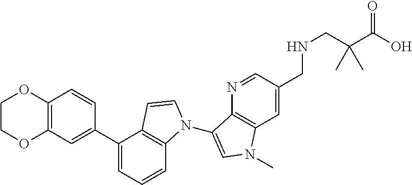 [SMILES: Cn1cc(-n2ccc3c(-c4ccc5c(c4)OCCO5)cccc32)c2ncc(CNCC(C)(C)C(=O)O)cc21]

A mixture of 3-(4-(2,3-dihydrobenzo[b][1,4]dioxin-6-yl)-1H-indol-1-yl)-1-methyl-1H-pyrrolo[3,2-b]pyridine-6-carbaldehyde (0.03 g, 0.07 mmol) and 3-amino-2,2-dimethylpropanoic acid (0.01 g, 0.09 mmol) in 4 mL MeOH/DCM (1:1) was stirred at room temperature and acetic acid was added until the pH was around 4. After the reaction was stirred for 1 hour, sodium cyanoborohydride (0.01 g, 0.22 mmol) was added and the reaction was stirred at room temperature for an additional 1 hour. The crude mixture was purified by column chromatography (silica gel, 10% MeOH/DCM). The fractions containing the product were collected and the solvent was removed under reduced pressure, resulting in the title compound. LCMS Method C, RT=3.7210 min, m/z=511.2 [M+H]⁺, LCMS Method D, RT=4.082 min, m/z=511.2312 [M+H]⁺, exact mass: 510.2267.

### Example 26: (S)-5-(((4-(4-(1-(3,5-dimethoxy-4-(((((S)-5-oxopyrrolidin-2-yl)methyl)amino) methyl)phenyl)-1H-indol-4-yl)indolin-1-yl)-2,6-dimethoxybenzyl)amino)methyl)pyrrolidin-2-one

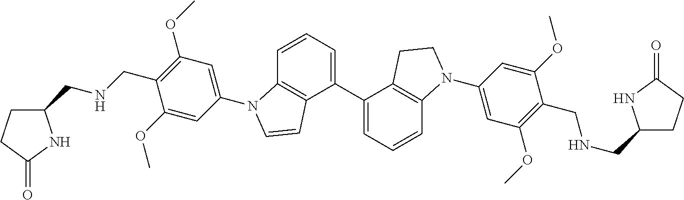 [SMILES: COc1cc(N2CCc3c(-c4cccc5c4ccn5-c4cc(OC)c(CNC[C@@H]5CCC(=O)N5)c(OC)c4)cccc32)cc(OC)c1CNC[C@@H]1CCC(=O)N1]

### Step 1: 4-(4,4,5,5-tetramethyl-1,3,2-dioxaborolan-2-yl)indoline

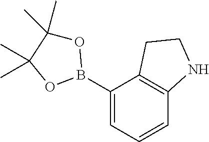 [SMILES: CC1(C)OB(c2cccc3c2CCN3)OC1(C)C]

Pd(dppf)Cl₂·CH₂·Cl₂ (0.20 g, 0.25 mmol) was added to a solution of 4-bromoindoline (1.00 g, 5.05 mmol), B₂Pin₂ (1.54 g, 6.06 mmol), and KOAc (3.42 g, 34.8 mmol) in DMF 15 mL. The resulting suspension was sparged with argon for 10 min. After the reaction was sealed and stirred at 150° C. for 1 hour under argon atmosphere, it was cooled down and diluted with water. The resulting mixture was extracted 3 times with EtOAc and the organic layer was collected, dried over anhydrous Na₂SO₄, filtered, and evaporated to dryness. The product was further used without purification.

### Step 2: 4-(indolin-4-yl)-1H-indole

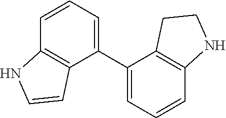 [SMILES: c1cc2c(c(-c3cccc4[nH]ccc34)c1)CCN2]

A mixture of 4-(4,4,5,5-tetramethyl-1,3,2-dioxaborolan-2-yl)indoline (1.24 g, 5.05 mmol), 4-bromo-1H-indole (0.82 g, 4.21 mmol), Pd(PPh₃)₄ (0.58 g, 0.51 mmol) and K₂CO₃ (1.16 g, 8.42 mmol) in a mixture of 1,4-dioxane and H₂O (6:1) 35 mL was sparged with argon for 10 min before stirring at 95° C. for 3 h under argon atmosphere. Then, the mixture was cooled down and diluted with brine. The resulting mixture was extracted 3 times with EtOAc and the organic layer was collected, dried over anhydrous Na₂SO₄, filtered, and evaporated to dryness. The crude mixture was purified by column chromatography (silica gel, gradient elution, 20 to 50% EtOAc/hexanes) to provide the product.

### Step 3: 4-(4-(1-(4-formyl-3,5-dimethoxyphenyl)-1H-indol-4-yl)indolin-1-yl)-2,6-dimethoxybenzaldehyde

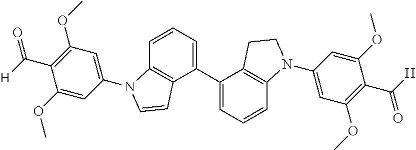 [SMILES: COc1cc(N2CCc3c(-c4cccc5c4ccn5-c4cc(OC)c(C=O)c(OC)c4)cccc32)cc(OC)c1C=O]

A mixture of 4-(indolin-4-yl)-1H-indole (0.28 g, 1.19 mmol), 4-bromo-2,6-dimethoxybenzaldehyde (0.32 g, 1.31 mmol), Pd(dba)₃ (0.06 g, 0.06 mmol), Xantphos (0.07 g, 0.12 mmol) and Cs₂CO₃ (1.36 g, 4.19 mmol) in toluene 15 mL was sparged with argon for 10 min before it was refluxed under argon atmosphere for 16 h. The resulting mixture was cooled down, filtered, and evaporated to dryness. The crude mixture was purified by column chromatography (silica gel, gradient elution, 20 to 80% EtOAc/hexanes).

### Step 4: (S)-5-(((4-(4-(1-(3,5-dimethoxy-4-(((((S)-5-oxopyrrolidin-2-yl)methyl)amino)methyl)phenyl)-1H-indol-4-yl)indolin-1-yl)-2,6-dimethoxybenzyl)amino)methyl)pyrrolidin-2-one

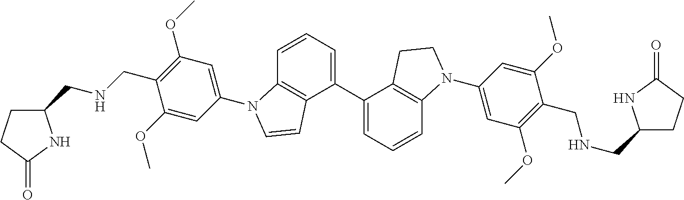 [SMILES: COc1cc(N2CCc3c(-c4cccc5c4ccn5-c4cc(OC)c(CNC[C@@H]5CCC(=O)N5)c(OC)c4)cccc32)cc(OC)c1CNC[C@@H]1CCC(=O)N1]

A mixture of 4-(4-(1-(4-formyl-3,5-dimethoxyphenyl)-1H-indol-4-yl)indolin-1-yl)-2,6-dimethoxybenzaldehyde (0.07 g, 0.17 mmol) and (S)-5-(aminomethyl)pyrrolidin-2-one (0.02 g, 0.20 mmol) in 4 mL MeOH/DCM (1:1) was stirred at room temperature and acetic acid was added until the pH was around 4. After the reaction was stirred for 1 hour, sodium cyanoborohydride (0.06 g, 1.00 mmol) was added and the reaction was stirred at room temperature for an additional 1 hour. The crude mixture was purified by column chromatography (silica gel, 10% MeOH/DCM). The fractions containing the product were collected and the solvent was removed under reduced pressure, resulting in the title compound. LCMS Method C, RT=3.8570 min, m/z=759.4 [M+H]⁺, LCMS Method D, RT=3.572 min, m/z=759.3754 [M+H]⁺, exact mass: 758.3800.

### Example 27: (S)-5-((((6-(2-chloro-3-(1-(3,5-dimethoxy-4-(((((S)-5-oxopyrrolidin-2-yl)methyl)amino) methyl)phenyl)-1H-indol-4-yl)phenyl)-2-methoxypyridin-3-yl)methyl)amino)methyl) pyrrolidin-2-one

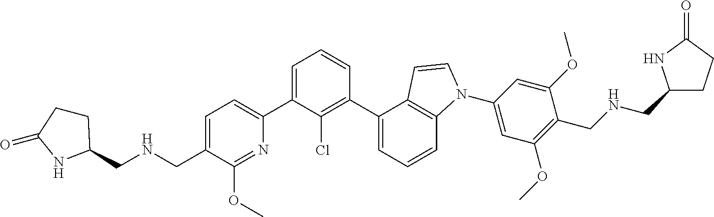 [SMILES: COc1cc(-n2ccc3c(-c4cccc(-c5ccc(CNC[C@@H]6CCC(=O)N6)c(OC)n5)c4Cl)cccc32)cc(OC)c1CNC[C@@H]1CCC(=O)N1]

A mixture of Intermediate D1 (96.0 mg, 0.18 mmol) and (S)-5-(aminomethyl)pyrrolidin-2-one (104 mg, 0.91 mmol) in 6 mL MeOH/DCM (1:1) was stirred at room temperature and acetic acid was added until the pH was around 4. After the reaction was stirred for 1 hour, sodium cyanoborohydride (57.20 mg, 0.91 mmol) was added and the reaction was stirred at room temperature for an additional 1 hour. The crude mixture was purified by column chromatography (silica gel, 10% MeOH/DCM). The fractions containing the product were collected and the solvent was removed under reduced pressure, resulting the title compound. LCMS Method C, RT=3.828 min, m/z=723.4 [M+H]⁺, LCMS Method D, RT=3.575 min, m/z=723.3053 [M+H]⁺, exact mass: 722.3000. ¹H NMR (500 MHz, MeOD-d₄) δ ppm 7.77 (d, J=7.5 Hz, 1H), 7.66 (d, J=8.4 Hz, 1H), 7.56 (dd, J=7.3, 2.0 Hz, 1H), 7.49 (d, J=3.4 Hz, 1H), 7.48-7.35 (m, 2H), 7.24 (m, 2H), 7.06 (d, J=7.2 Hz, 2H), 6.35 (d, J=3.3 Hz, 1H), 3.53 (s, 3H), 3.88 (s, 8H), 3.47 (q, 4H), 3.29-3.25 (m, 1H), 2.96-2.83 (m, 2H), 2.42-2.21 (m, 7H), 1.89-1.78 (m, 2H).

### Example 28: 1-((6-(2-chloro-3-(1-(4-((3-hydroxy-3-methylazetidin-1-yl)methyl)-3,5-dimethoxyphenyl)-1H-indol-4-yl)phenyl)-2-methoxypyridin-3-yl)methyl)-3-methylazetidin-3-ol

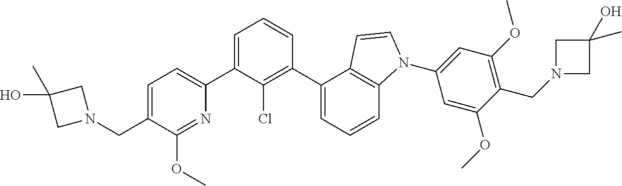 [SMILES: COc1cc(-n2ccc3c(-c4cccc(-c5ccc(CN6CC(C)(O)C6)c(OC)n5)c4Cl)cccc32)cc(OC)c1CN1CC(C)(O)C1]

A mixture of Intermediate D1 (52.70 mg, 0.1 mmol) and 3-methylazetidin-3-ol hydrochloride (14.80 mg, 0.12 mmol) in 4 mL MeOH/DCM (1:1) was stirred at room temperature and acetic acid was added until the pH was around 4. After the reaction was stirred for 1 hour, sodium cyanoborohydride (25.20 mg, 0.4 mmol) was added and the reaction was stirred at room temperature for an additional 1 hour. The crude mixture was purified by column chromatography (silica gel, gradient elution, 5 to 20% MeOH/DCM). The fractions containing the product were collected and the solvent was removed under reduced pressure, resulting in the title compound. LCMS Method C, RT=4.054 min, m/z=669.3 [M+H]⁺, LCMS Method D, RT=3.754 min, m/z=669.2828 [M+H]⁺, exact mass: 668.2800.

### Example 29: (S)-5-((((6-(2-chloro-3-(1-(3,5-dimethoxy-4-(((((S)-5-oxopyrrolidin-2-yl)methyl)amino)methyl)phenyl)-1H-indazol-4-yl)phenyl)-2-methoxypyridin-3-yl)methyl)amino)methyl)pyrrolidin-2-one

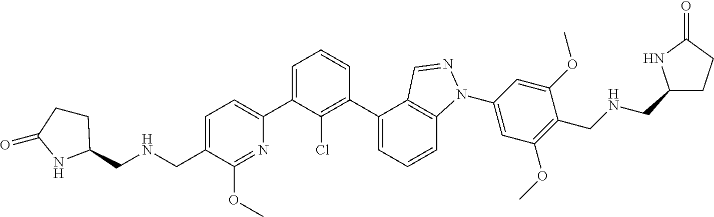 [SMILES: COc1cc(-n2ncc3c(-c4cccc(-c5ccc(CNC[C@@H]6CCC(=O)N6)c(OC)n5)c4Cl)cccc32)cc(OC)c1CNC[C@@H]1CCC(=O)N1]

A mixture of Intermediate D2 (29.4 mg, 0.06 mmol) and (S)-5-(aminomethyl)pyrrolidin-2-one (19.5 mg, 0.17 mmol) in 2 mL MeOH/DCM (1:1) was stirred at room temperature and acetic acid was added until the pH was around 4. After the reaction was stirred for 1 hour, sodium cyanoborohydride (40.6 mg, 0.59 mmol) was added and the reaction was stirred at room temperature for an additional 1 hour. The crude mixture was purified by column chromatography (silica gel, gradient elution, 0 to 50% MeOH/DCM). The fractions containing the product were collected and the solvent was removed under reduced pressure, resulting in the title compound. LCMS Method B, RT=3.065 min, m/z=724.4 [M+H]⁺, LCMS Method D, RT=3.469 min, m/z=724.2971 [M+H]⁺, exact mass: 723.2936.

Example 30: (S)-5-((((6-(2-chloro-3-(1-(3,5-dimethoxy-4-(((((S)-5-oxopyrrolidin-2-yl)methyl)amino)methyl)phenyl)-1H-pyrrolo[2,3-c]pyridin-4-yl)phenyl)-2-methoxypyridin-3-yl)methyl)amino)methyl)pyrrolidin-2-one

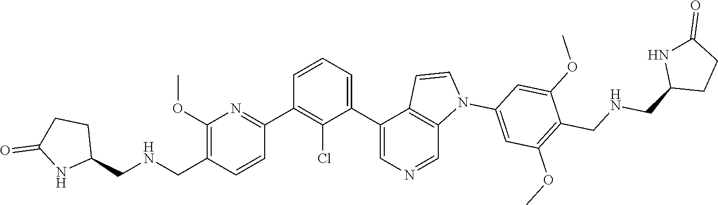 [SMILES: COc1cc(-n2ccc3c(-c4cccc(-c5ccc(CNC[C@@H]6CCC(=O)N6)c(OC)n5)c4Cl)cncc32)cc(OC)c1CNC[C@@H]1CCC(=O)N1]

### Step 1: 4-(4-bromo-1H-pyrrolo[2,3-c]pyridin-1-yl)-2,6-dimethoxybenzaldehyde

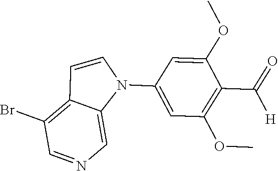 [SMILES: COc1cc(-n2ccc3c(Br)cncc32)cc(OC)c1C=O]

A mixture of 4-bromo-2,6-dimethoxybenzaldehyde (0.74 g, 3.1 mmol), 4-Bromo-1H-pyrrolo[2,3-c]pyridine (0.50 g, 2.54 mmol), CuO (10 mg, 0.13 mmol), and Cs₂CO₃ (1.66 g, 9.10 mmol) was suspended in DMSO 10 mL and sparged with argon for 10 min before stirring at 100° C. for overnight. The mixture was cooled and diluted with EtOAc. The reaction was extracted with EtOAc/H₂O. The organic layer was washed with water and brine. The organic layers were collected, dried over anhydrous Na₂SO₄, filtered, and evaporated to dryness. The crude mixture was purified by column chromatography (silica gel, gradient elution, 20 to 50% EtOAc/hexanes) to provide the product.

### Step 2: 2,6-dimethoxy-4-(4-(4,4,5,5-tetramethyl-1,3,2-dioxaborolan-2-yl)-1H-pyrrolo[2,3-c]pyridin-1-yl)benzaldehyde

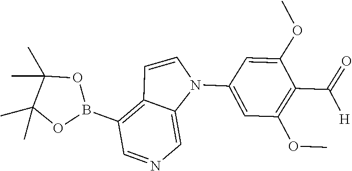 [SMILES: COc1cc(-n2ccc3c(B4OC(C)(C)C(C)(C)O4)cncc32)cc(OC)c1C=O]

Pd(dppf)Cl₂·CH₂CO₂ (8.0 mg, 0.01 mmol) was added to a solution of 4-(4-bromo-1H-pyrrolo[2,3-c]pyridin-1-yl)-2,6-dimethoxybenzaldehyde (80 mg, 0.20 mmol), bis(pinacolato)diboron (60 mg, 0.25 mmol), and KOAc (0.14 g, 1.4 mmol) in DMF 3 mL. The resulting suspension was sparged with argon for 10 min. After the reaction was sealed and stirred at 150° C. for 1 hour under argon atmosphere, it was cooled down and diluted with water. The resulting mixture was extracted 3 times with EtOAc and the organic layer was collected, dried over anhydrous Na₂SO₄, filtered, and evaporated to dryness. The product was further used without purification.

### Step 3: 6-(2-chloro-3-(1-(4-formyl-3,5-dimethoxyphenyl)-1H-pyrrolo[2,3-c]pyridin-4-yl)phenyl)-2-methoxynicotinaldehyde

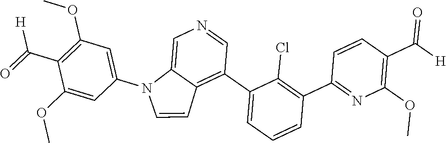 [SMILES: COc1cc(-n2ccc3c(-c4cccc(-c5ccc(C=O)c(OC)n5)c4Cl)cncc32)cc(OC)c1C=O]

A mixture of 2,6-dimethoxy-4-(4-(4,4,5,5-tetramethyl-1,3,2-dioxaborolan-2-yl)-1H-pyrrolo[2,3-c]pyridin-1-yl)benzaldehyde (0.27 mmol of crude mixture), Intermediate C1 (55 mg, 0.17 mmol), Pd(PPh₃)₄ (0.06 g, 0.03 mmol) and K₂CO₃ (70 mg, 0.7 mmol) in a mixture of 1,4-dioxane and H₂O (6:1) 5 mL was sparged with argon for 10 min before stirring at 95° C. for 30 min under argon atmosphere. Then, the mixture was cooled down and diluted with brine. The resulting mixture was extracted 3 times with EtOAc and the organic layer was collected, dried over anhydrous Na₂SO₄, filtered, and evaporated to dryness. The crude mixture was purified by column chromatography (silica gel, gradient elution, 50 to 100% EtOAc/hexanes) to provide the product.

Step 4: (S)-5-((((6-(2-chloro-3-(1-(3,5-dimethoxy-4-(((((S)-5-oxopyrrolidin-2-yl)methyl)amino)methyl)phenyl)-1H-pyrrolo[2,3-c]pyridin-4-yl)phenyl)-2-methoxypyridin-3-yl)methyl)amino)methyl)pyrrolidin-2-one

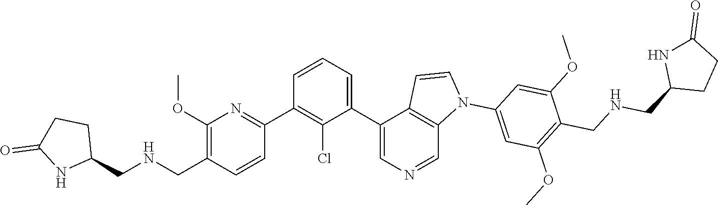 [SMILES: COc1cc(-n2ccc3c(-c4cccc(-c5ccc(CNC[C@@H]6CCC(=O)N6)c(OC)n5)c4Cl)cncc32)cc(OC)c1CNC[C@@H]1CCC(=O)N1]

A mixture of 6-(2-chloro-3-(1-(4-formyl-3,5-dimethoxyphenyl)-1H-pyrrolo[2,3-c]pyridin-4-yl)phenyl)-2-methoxynicotinaldehyde (60 mg, 0.11 mmol) and (S)-5-(aminomethyl)pyrrolidin-2-one (27 mg, 0.24 mmol) in 6 mL MeOH/DCM (1:1) was stirred at room temperature and acetic acid was added until the pH was around 4. After the reaction was stirred for 1 hour, sodium cyanoborohydride (63 mg, 0.55 mmol) was added and the reaction was stirred at room temperature for an additional 1 hour. The crude mixture was purified by column chromatography (silica gel, gradient elution, 10 to 15% MeOH/DCM). The fractions containing the product were collected and the solvent was removed under reduced pressure, resulting in the title compound. LCMS Method A, RT=2.943 min, m/z=724.3 [M+H]⁺, LCMS Method D, RT=2.571 min, m/z=724.3043 [M+H]⁺, exact mass: 723.2900.

Example 31: (S)-5-((((6-(2-chloro-3-(3-(3,5-dimethoxy-4-(((((S)-5-oxopyrrolidin-2-yl)methyl)amino)methyl)phenyl)-1-methyl-1H-indazol-7-yl)phenyl)-2-methoxypyridin-3-yl)methyl)amino)methyl)pyrrolidin-2-one

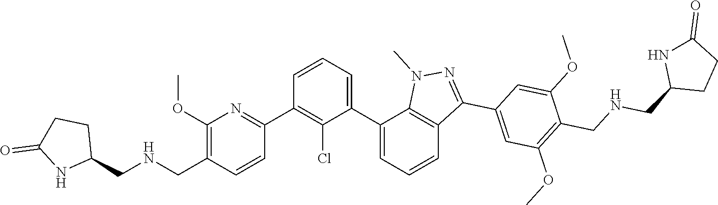 [SMILES: COc1cc(-c2nn(C)c3c(-c4cccc(-c5ccc(CNC[C@@H]6CCC(=O)N6)c(OC)n5)c4Cl)cccc23)cc(OC)c1CNC[C@@H]1CCC(=O)N1]

### Step 1: 6-(2-chloro-3-(3-(4-formyl-3,5-dimethoxyphenyl)-1-methyl-1H-indazol-7-yl)phenyl)-2-methoxynicotinaldehyde

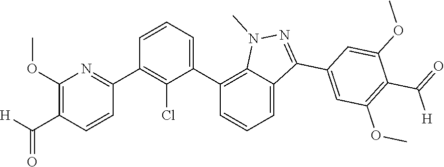 [SMILES: COc1cc(-c2nn(C)c3c(-c4cccc(-c5ccc(C=O)c(OC)n5)c4Cl)cccc23)cc(OC)c1C=O]

A mixture of Intermediate B7 (0.15 g, 0.27 mmol), Intermediate C2 (0.432 g, 0.32 mmol), Pd(PPh₃)₄ (0.08 g, 0.04 mmol) and K₂CO₃ (0.20 g, 2.1 mmol) in a mixture of 1,4-dioxane and H₂O (6:1) 5 mL was sparged with argon for 10 min before stirring at 95° C. for 30 min under argon atmosphere. Then, the mixture was cooled down and diluted with brine. The resulting mixture was extracted 3 times with EtOAc and the organic layer was collected, dried over anhydrous Na₂SO₄, filtered, and evaporated to dryness. The crude mixture was purified by column chromatography (silica gel, gradient elution, 50 to 100% EtOAc/hexanes) to provide the product.

Step 2: (S)-5-((((6-(2-chloro-3-(3-(3,5-dimethoxy-4-(((((S)-5-oxopyrrolidin-2-yl)methyl)amino)methyl)phenyl)-1-methyl-1H-indazol-7-yl)phenyl)-2-methoxypyridin-3-yl)methyl)amino)methyl)pyrrolidin-2-one

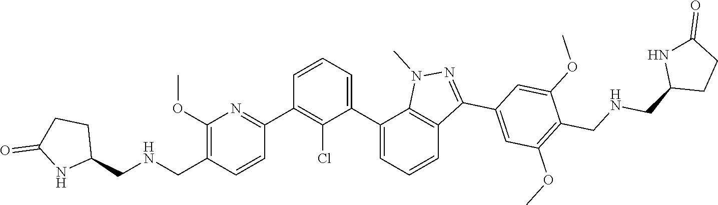 [SMILES: COc1cc(-c2nn(C)c3c(-c4cccc(-c5ccc(CNC[C@@H]6CCC(=O)N6)c(OC)n5)c4Cl)cccc23)cc(OC)c1CNC[C@@H]1CCC(=O)N1]

A mixture of 6-(2-chloro-3-(3-(4-formyl-3,5-dimethoxyphenyl)-1-methyl-1H-indazol-7-yl)phenyl)-2-methoxynicotinaldehyde (30 mg, 0.06 mmol) and (S)-5-(aminomethyl)pyrrolidin-2-one (16 mg, 0.14 mmol) in 4 mL MeOH/DCM (1:1) was stirred at room temperature and acetic acid was added until the pH was around 4. After the reaction was stirred for 1 hour, sodium cyanoborohydride (33 mg, 0.30 mmol) was added and the reaction was stirred at room temperature for an additional 1 hour. The crude mixture was purified by column chromatography (silica gel, gradient elution, 10 to 15% MeOH/DCM). The fractions containing the product were collected and the solvent was removed under reduced pressure, resulting in the title compound. LCMS Method A, RT=3.047 min, m/z=738.4 [M+H]⁺, LCMS Method D, RT=3.525 min, m/z=738.3164 [M+H]⁺, exact mass: 737.3000.

### Example 32: (S)-5-(((4-(4-(3-(5-((6-acetyl-2,6-diazaspiro[3.3]heptan-2-yl)methyl)-6-methoxypyridin-2-yl)-2-chlorophenyl)-1H-indol-1-yl)-2,6-dimethoxybenzyl)amino)methyl) pyrrolidin-2-one

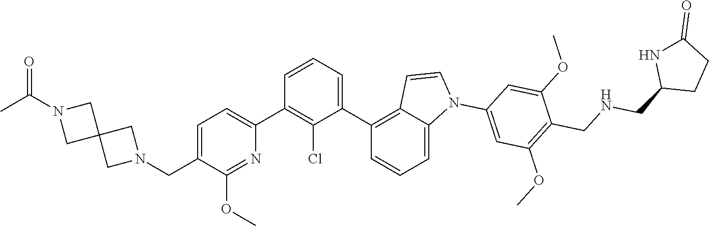 [SMILES: COc1cc(-n2ccc3c(-c4cccc(-c5ccc(CN6CC7(C6)CN(C(C)=O)C7)c(OC)n5)c4Cl)cccc32)cc(OC)c1CNC[C@@H]1CCC(=O)N1]

### Step 1: 1-(6-((6-(3-bromo-2-chlorophenyl)-2-methoxypyridin-3-yl)methyl)-2,6-diazaspiro[3.3]heptan-2-yl)ethan-1-one

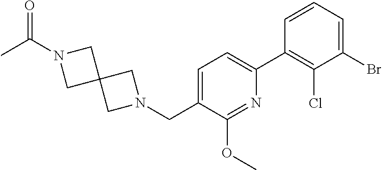 [SMILES: COc1nc(-c2cccc(Br)c2Cl)ccc1CN1CC2(C1)CN(C(C)=O)C2]

A mixture of Intermediate C1 (0.15 g, 0.50 mmol) and ter-butyl 2,6-diazaspiro[3.3]heptane-2-carboxylate (0.22 g, 0.50 mmol) in 10 mL MeOH/DCM (1:1) was stirred at room temperature and acetic acid was added until the pH was around 4. After the reaction was stirred for 1 hour, sodium cyanoborohydride (0.16 g, 2.50 mmol) was added and the reaction was stirred at room temperature for an additional 1 hour. The crude mixture was purified by column chromatography (silica gel, 10% MeOH/DCM). The fractions containing the product were collected and the solvent was removed under reduced pressure. The resulting oil was treated with 50% trifluoroacetic acid in DCM and stirred at room temperature for 2 h. After evaporation to dryness, the resulting crude mixture was stirred at room temperature in the presence of acetic anhydride (0.14 mL, 1.50 mmol) and triethylamine (0.21 mL, 1.50 mmol). After 16 h, the resulting mixture was extracted 3 times with EtOAc and the organic layer was collected, dried over anhydrous Na₂SO₄, filtered, and evaporated to dryness. The desired product was further used without purification.

### Step 2: 4-(4-(3-(5-((6-acetyl-2,6-diazaspiro[3.3]heptan-2-yl)methyl)-6-methoxypyridin-2-yl)-2-chlorophenyl)-1H-indol-1-yl)-2,6-dimethoxybenzaldehyde

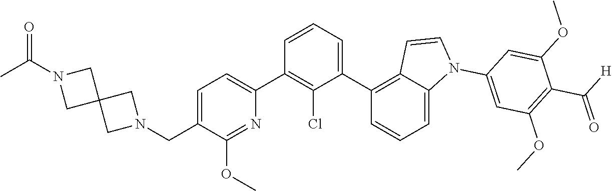 [SMILES: COc1cc(-n2ccc3c(-c4cccc(-c5ccc(CN6CC7(C6)CN(C(C)=O)C7)c(OC)n5)c4Cl)cccc32)cc(OC)c1C=O]

A mixture of 1-(6-((6-(3-bromo-2-chlorophenyl)-2-methoxypyridin-3-yl)methyl)-2,6-diazaspiro[3.3]heptan-2-yl)ethan-1-one (0.23 g, 0.50 mmol), Intermediate B1-1 (0.24 g, 0.60 mmol), Pd(PPh₇)₄ (0.06 g, 0.05 mmol) and K₂CO₃ (0.17 g, 1.20 mmol) in a mixture of 1,4-dioxane and H₂O (6:1) 14 mL was sparged with argon for 10 min before stirring at 95° C. for 3 h under argon atmosphere. Then, the mixture was cooled down and diluted with brine. The resulting mixture was extracted 3 times with EtOAc and the organic layer was collected, dried over anhydrous Na₂SO₄, filtered, and evaporated to dryness. The crude mixture was purified by column chromatography (silica gel, gradient elution, 50 to 100% EtOAc/hexanes).

### Step 3: (S)-5-(((4-(4-(3-(5-((6-acetyl-2,6-diazaspiro[3.3]heptan-2-yl)methyl)-6-methoxypyridin-2-yl)-2-chlorophenyl)-1H-indol-1-yl)-2,6-dimethoxybenzyl)amino)methyl) pyrrolidin-2-one

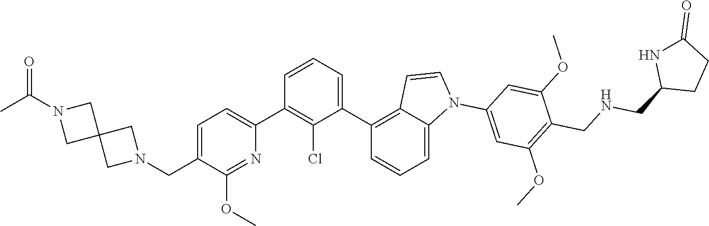 [SMILES: COc1cc(-n2ccc3c(-c4cccc(-c5ccc(CN6CC7(C6)CN(C(C)=O)C7)c(OC)n5)c4Cl)cccc32)cc(OC)c1CNC[C@@H]1CCC(=O)N1]

A mixture of 4-(4-(3-(5-((6-acetyl-2,6-diazaspiro[3.3]heptan-2-yl)methyl)-6-methoxypyridin-2-yl)-2-chlorophenyl)-1H-indol-1-yl)-2,6-dimethoxybenzaldehyde (0.13 g, 0.20 mmol) and (S)-5-(aminomethyl)pyrrolidin-2-one (0.07 g, 0.60 mmol) in 4 mL MeOH/DCM (1:1) was stirred at room temperature and acetic acid was added until the pH was around 4. After the reaction was stirred for 1 hour, sodium cyanoborohydride (0.06 g, 1.00 mmol) was added and the reaction was stirred at room temperature for an additional 1 hour. The crude mixture was purified by column chromatography (silica gel, gradient elution, 10 to 15% MeOH/DCM). The fractions containing the product were collected and the solvent was removed under reduced pressure, resulting the title compound. LCMS Method C, RT=3.913 min, m/z=749.4 [M+H]⁺771.4 [M+Na]⁺, LCMS Method D, RT=3.632 min, m/z=749.3207 [M+H]⁺, exact mass: 748.3100.

### Example 33: (S)-5-(((4-(4-(3-(5-(((azetidin-3-ylmethyl)amino)methyl)-6-methoxypyridin-2-yl)-2-chlorophenyl)-1H-indol-1-yl)-2,6-dimethoxybenzyl)amino)methyl)pyrrolidin-2-one

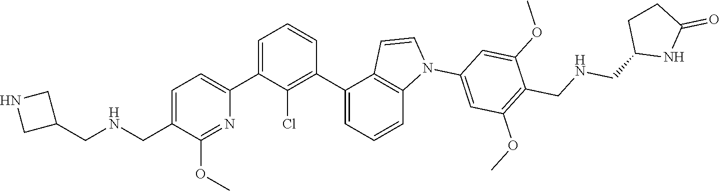 [SMILES: COc1cc(-n2ccc3c(-c4cccc(-c5ccc(CNCC6CNC6)c(OC)n5)c4Cl)cccc32)cc(OC)c1CNC[C@@H]1CCC(=O)N1]

A mixture of Intermediate F1 (50 mg, 0.07 mmol) and tert-butyl 3-(aminomethyl)azetidine-1-carboxylate (14.90 mg, 0.08 mmol) in 6 mL MeOH/DCM (1:1) was stirred at room temperature and acetic acid was added until the pH was around 4. After the reaction was stirred for 1 hour, sodium cyanoborohydride (21.99 mg, 0.35 mmol) was added and the reaction was stirred at room temperature for an additional 1 hour. The crude mixture was further used without purification.

The residue was added with 4M HCl in dioxane (1 mL) and stirred at room temperature for 30 minutes. The residual solvent was removed under reduced pressure and the residue was washed with dioxane. The crude mixture was purified by Sephadex column chromatography (100% MeOH), affording the title compound. LCMS method C, RT=3.820 min, m/z=695.4 [M+H]⁺, LCMS Method D, RT=3.289 min, m/z=695.3108 [M+H]⁺, exact mass. 694.3034.

### Example 34: (S)-5-(((4-(4-(2-chloro-3-(5-(((R)-3-hydroxypyrrolidin-1-yl)methyl)-6-methoxypyridin-2-yl)phenyl)-1H-pyrrolo[2,3-b]pyridin-1-yl)-2,6-dimethoxybenzyl)amino) methyl)pyrrolidin-2-one

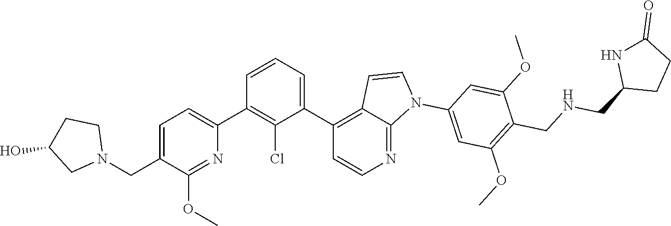 [SMILES: COc1cc(-n2ccc3c(-c4cccc(-c5ccc(CN6CC[C@@H](O)C6)c(OC)n5)c4Cl)ccnc32)cc(OC)c1CNC[C@@H]1CCC(=O)N1]

### Step 1: (R)-1-((6-(3-bromo-2-chlorophenyl)-2-methoxypyridin-3-yl)methyl)pyrrolidin-3-ol

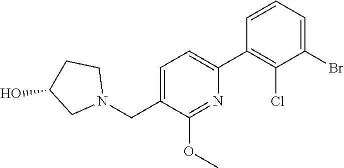 [SMILES: COc1nc(-c2cccc(Br)c2Cl)ccc1CN1CC[C@@H](O)C1]

A mixture of Intermediate C1 (0.30 g, 0.92 mmol) and (R)-pyrrolidin-3-ol (0.34 g, 2.76 mmol) in 8 mL MeOH/DCM (1:1) was stirred at room temperature and acetic acid was added until the pH was around 4. After the reaction was stirred for 1 hour, sodium cyanoborohydride (0.29 g, 4.60 mmol) was added and the reaction was stirred at room temperature for an additional 1 hour. The crude mixture was purified by column chromatography (silica gel, gradient elution, 10 to 15% MeOH/DCM). The fractions containing the product were collected and the solvent was removed under reduced pressure.

### Step 2: (R)-4-(4-(2-chloro-3-(5-((3-hydroxypyrrolidin-1-yl)methyl)-6-methoxypyridin-2-yl)phenyl)-1H-pyrrolo[2,3-b]pyridin-1-yl)-2,6-dimethoxybenzaldehyde

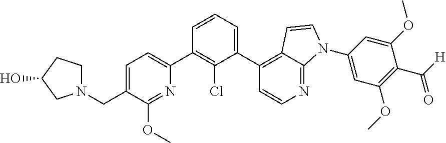 [SMILES: COc1cc(-n2ccc3c(-c4cccc(-c5ccc(CN6CC[C@@H](O)C6)c(OC)n5)c4Cl)ccnc32)cc(OC)c1C=O]

A mixture of (R)-1-((6-(3-bromo-2-chlorophenyl)-2-methoxypyridin-3-yl)methyl)pyrrolidin-3-ol (0.28 g, 0.70 mmol), Intermediate B3-1 (0.33 g, 0.80 mmol), Pd(PPh₃)₄ (0.16 g, 0.14 mmol) and K₂CO₃ (2.32 g, 16.8 mmol) in a mixture of 1,4-dioxane and H₂O (6:1) 14 mL was sparged with argon for 10 min before stirring at 95° C. for 3 h under argon atmosphere. Then, the mixture was cooled down and diluted with brine. The resulting mixture was extracted 3 times with EtOAc and the organic layer was collected, dried over anhydrous Na₂SO₄, filtered, and evaporated to dryness. The crude mixture was purified by column chromatography (silica gel, gradient elution, 5 to 10% MeOH/DCM).

### Step 3: (S)-5-(((4-(4-(2-chloro-3-(5-(((R)-3-hydroxypyrrolidin-1-yl)methyl)-6-methoxypyridin-2-yl)phenyl)-1H-pyrrolo[2,3-b]pyridin-1-yl)-2,6-dimethoxybenzyl)amino) methyl)pyrrolidin-2-one

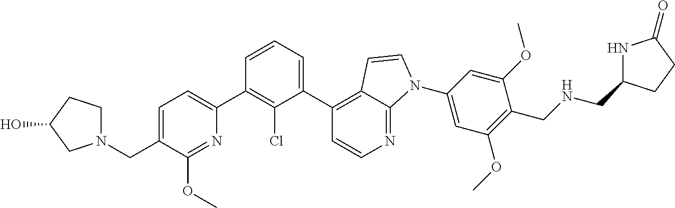 [SMILES: COc1cc(-n2ccc3c(-c4cccc(-c5ccc(CN6CC[C@@H](O)C6)c(OC)n5)c4Cl)ccnc32)cc(OC)c1CNC[C@@H]1CCC(=O)N1]

A mixture of (R)-4-(4-(2-chloro-3-(5-((3-hydroxypyrrolidin-1-yl)methyl)-6-methoxypyridin-2-yl)phenyl)-1H-pyrrolo[2,3-b]pyridin-1-yl)-2,6-dimethoxybenzaldehyde (0.12 g, 0.20 mmol) and (S)-5-(aminomethyl)pyrrolidin-2-one (0.03 g, 0.24 mmol) in 4 mL MeOH/DCM (1:1) was stirred at room temperature and acetic acid was added until the pH was around 4. After the reaction was stirred for 1 hour, sodium cyanoborohydride (0.06 g, 1.00 mmol) was added and the reaction was stirred at room temperature for an additional 1 hour. The crude mixture was purified by column chromatography (silica gel, gradient elution, 10 to 15% MeOH/DCM). The fractions containing the product were collected and the solvent was removed under reduced pressure, resulting the title compound. LCMS Method C, RT=3.903 min, m/z=697.3 [M+H]⁺, LCMS Method D, RT=3.484 min, m/z=697.2897 [M+H]⁺, exact mass: 696.2800.

### Example 35: (S)-5-(((4-(4-(2-chloro-3-(5-((3-hydroxy-3-methylazetidin-1-yl)methyl)-6-methoxypyridin-2-yl)phenyl)-1H-indol-1-yl)-2,6-dimethoxybenzyl)amino)methyl)pyrrolidin-2-one

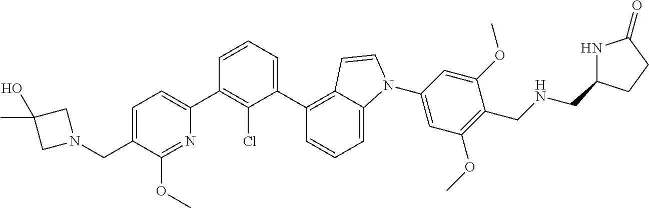 [SMILES: COc1cc(-n2ccc3c(-c4cccc(-c5ccc(CN6CC(C)(O)C6)c(OC)n5)c4Cl)cccc32)cc(OC)c1CNC[C@@H]1CCC(=O)N1]

A mixture of Intermediate F1 (0.12 g, 0.17 mmol) and 3-methylazetidin-3-ol (0.02 g, 0.20 mmol) in 4 ml, MeOH/DCM (1.1) was stirred at room temperature and acetic acid was added until the pH was around 4. After the reaction was stirred for 1 hour, sodium cyanoborohydride (0.03 g, 0.51 mmol) was added and the reaction was stirred at room temperature for an additional 1 hour. After evaporation to dryness, the resulting crude mixture was stirred at room temperature in the presence of 4M HCl in dioxane for 30 min. All solvent was evaporated under vacuum and the crude mixture was purified by column chromatography (silica gel, gradient elution, 5 to 10% MeOH/DCM). The fractions containing the product were collected and the solvent was removed under reduced pressure, resulting in the title compound. LCMS Method C, RT=3.980 min, m/z=696.4 [M+H]⁺, LCMS Method D, RT=3.662 min, m/z=696.2939 [M+H]⁺, exact mass: 695.2874.

Example 36: (S)-5-((((6-(2-chloro-3-(1-(4-((6-hydroxy-6-methyl-2-azaspiro[3.3]heptan-2-yl)methyl)-3,5-dimethoxyphenyl)-1H-pyrrolo[2,3-b]pyridin-4-yl)phenyl)-2-methoxypyridin-3-yl)methyl)amino)methyl)pyrrolidin-2-one

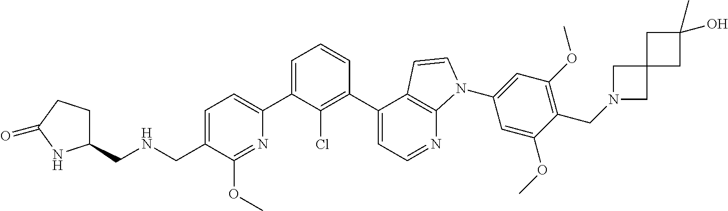 [SMILES: COc1cc(-n2ccc3c(-c4cccc(-c5ccc(CNC[C@@H]6CCC(=O)N6)c(OC)n5)c4Cl)ccnc32)cc(OC)c1CN1CC2(C1)CC(C)(O)C2]

A mixture of Intermediate E2 (72.50 mg, 0.10 mmol) and 6-Methyl-2-azaspiro[3.3]heptan-6-ol hydrochloride (32.70 mg, 0.20 mmol) in 4 mL MeOH/DCM (1:1) was stirred at room temperature and acetic acid was added until the pH was around 4. After the reaction was stirred for 1 hour, sodium cyanoborohydride (50.4 mg, 0.80 mmol) was added and the reaction was stirred at room temperature for an additional 1 hour. After evaporation to dryness, the resulting crude mixture was stirred at room temperature in the presence of 4M HCl in dioxane for 30 min. All solvent was evaporated under vacuum and the crude mixture was purified by column chromatography (silica gel, gradient elution, 5 to 25% MeOH/DCM). The fractions containing the product were collected and the solvent was removed under reduced pressure, resulting in the title compound. LCMS Method C, RT=3.967 min, m/z=737.3 [M+H]⁺, LCMS Method D, RT=3.596 min, m/z=737.3201 [M+H]⁺, exact mass: 736.3140.

### Example 37: (S)-1-((6-(2-chloro-3-(1-(3,5-dimethoxy-4-((((5-oxopyrrolidin-2-yl)methyl)amino)methyl)phenyl)-1H-indol-4-yl)phenyl)-2-methoxypyridin-3-yl)methyl)-3-methylazetidine-3-carboxylic acid

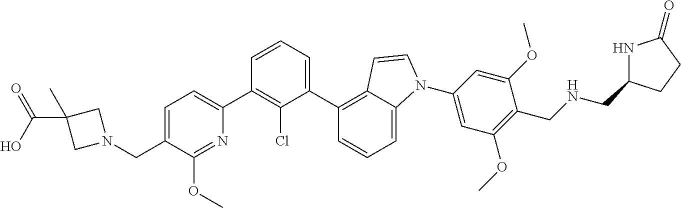 [SMILES: COc1cc(-n2ccc3c(-c4cccc(-c5ccc(CN6CC(C)(C(=O)O)C6)c(OC)n5)c4Cl)cccc32)cc(OC)c1CNC[C@@H]1CCC(=O)N1]

A mixture of Intermediate F1 (0.12 g, 0.17 mmol) and 3-methylazetidine-3-carboxylic acid (0.02 g, 0.20 mmol) in 4 mL MeOH/DCM (1:1) was stirred at room temperature and acetic acid was added until the pH was around 4. After the reaction was stirred for 1 hour, sodium cyanoborohydride (0.03 g, 0.51 mmol) was added and the reaction was stirred at room temperature for an additional 1 hour. After evaporation to dryness, the resulting crude mixture was stirred at room temperature in the presence of 4M HCl in dioxane for 30 min. All solvent was evaporated under vacuum and the crude mixture was purified by column chromatography (silica gel, gradient elution, 5 to 20% MeOH/DCM). The fractions containing the product were collected and the solvent was removed under reduced pressure, resulting in the title compound. LCMS Method C, RT=4.036 min, m/z=724.3 [M+H]⁺, LCMS Method D, RT=3.742 min, m/z=724.2897 [M+H]⁺, exact mass: 723.2800.

### Example 38: N—((R)-1-(4-(4-(2-chloro-3-(6-methoxy-5-(((((S)-5-oxopyrrolidin-2-yl)methyl)amino)methyl)pyridin-2-yl)phenyl)-1H-indol-1-yl)-2,6-dimethoxybenzyl)pyrrolidin-3-yl)acetamide

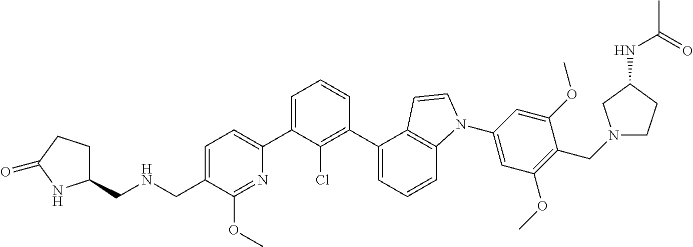 [SMILES: COc1cc(-n2ccc3c(-c4cccc(-c5ccc(CNC[C@@H]6CCC(=O)N6)c(OC)n5)c4Cl)cccc32)cc(OC)c1CN1CC[C@@H](NC(C)=O)C1]

### Step 1: (R)—N-(pyrrolidin-3-yl)acetamide hydrochloride

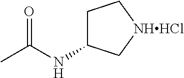

A mixture of tert-butyl (R)-3-aminopyrrolidine-1-carboxylate (0.50 g, 2.68 mmol), acetic anhydride (278 μL, 2.95 mmol) and triethylamine (1.12 mL, 8.04 mmol) in THF (20 mL) was stirred at room temperature for 30 minutes. Then, the residue was dissolved in dioxane and 4M HCl (1 mL) was added. After the reaction was stirred at 60° C. for 1 hour, the solvent was removed under reduced pressure and the residue was washed with dioxane to provide (R)—N-(pyrrolidin-3-yl)acetamide hydrochloride.

### Step 2: N—((R)-1-(4-(4-(2-chloro-3-(6-methoxy-5-(((((S)-5-oxopyrrolidin-2-yl)methyl)amino)methyl)pyridin-2-yl)phenyl)-1H-indol-1-yl)-2,6-dimethoxybenzyl)pyrrolidin-3-yl)acetamide

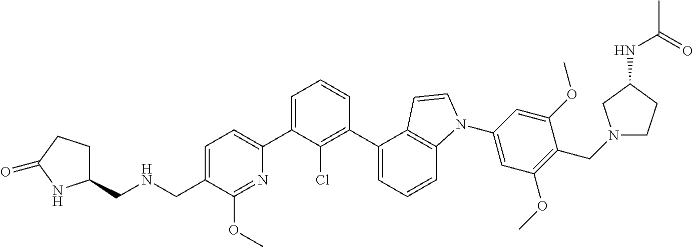 [SMILES: COc1cc(-n2ccc3c(-c4cccc(-c5ccc(CNC[C@@H]6CCC(=O)N6)c(OC)n5)c4Cl)cccc32)cc(OC)c1CN1CC[C@@H](NC(C)=O)C1]

A mixture of Intermediate E1 (156 mg, 0.21 mmol) and (R)—N-(pyrrolidin-3-yl)acetamide hydrochloride (55.57 mg, 0.43 mmol) in MeOH:DCM (5:3, 5 mL) was stirred at room temperature and acetic acid was added until the pH was around 4. After the reaction was stirred for 1 hour, sodium cyanoborohydride (67.56 mg, 1.08 mmol) was added. The reaction was stirred at room temperature for an additional 1 hour, quenched by a small amount of MeOH and evaporated to dryness. Then, the residue was dissolved in dioxane and 4M HCl (1 mL) was added. After the reaction was stirred at room temperature for 30 minutes, the residual solvent was removed under reduced pressure and the residue was washed with dioxane. The crude mixture was purified by Sephadex column chromatography (100% MeOH) to provide the title compound. LCMS method C, RT=4.019 min, m/z=737.3 [M+H]⁺, LCMS Method D, RT=3.674 min, m/z=737.3215 [M+H]⁺, exact mass: 736.3140.

### Example 39: (S)-3-((4-(4-(2-chloro-3-(6-methoxy-5-((((5-oxopyrrolidin-2-yl)methyl)amino)methyl)pyridin-2-yl)phenyl)-1H-indol-1-yl)-2,6-dimethoxybenzyl)amino)-2,2-dimethylpropanamide

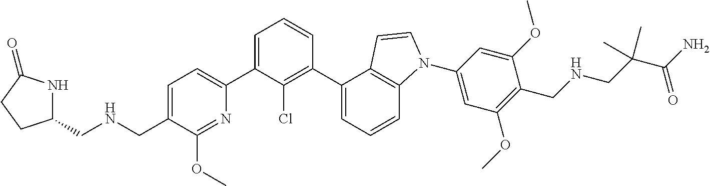 [SMILES: COc1cc(-n2ccc3c(-c4cccc(-c5ccc(CNC[C@@H]6CCC(=O)N6)c(OC)n5)c4Cl)cccc32)cc(OC)c1CNCC(C)(C)C(N)=O]

A mixture of Intermediate E1 (50 mg, 0.07 mmol) and 3-amino-2,2-dimethylpropanamide (9.29 mg, 0.08 mmol) in 6 mL MeOH/DCM (1:1) was stirred at room temperature and acetic acid was added until the pH was around 4. After the reaction was stirred for 1 hour, sodium cyanoborohydride (21.99 mg, 0.35 mmol) was added and the reaction was stirred at room temperature for an additional 1 hour. The crude mixture was further used without purification.

The residue was added with 4M HCl in dioxane (1 mL) and stirred at room temperature for 30 minutes. The residual solvent was removed under reduced pressure and the residue was washed with dioxane. The crude mixture was purified by Sephadex column chromatography (100% MeOH), affording the title compound. LCMS method C, RT=3.960 min, m/z=725.4 [M+H]⁺, LCMS Method D, RT=3.707 min, m/z=725.3209 [M+H]⁺, exact mass: 724.3140.

### Example 40: (S)-1-(4-(4-(2-chloro-3-(6-methoxy-5-((((5-oxopyrrolidin-2-yl)methyl)amino)methyl)pyridin-2-yl)phenyl)-1H-indol-1-yl)-2,6-dimethoxybenzyl)-3-methylazetidine-3-carboxylic acid

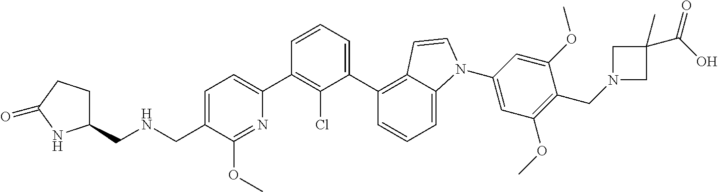 [SMILES: COc1cc(-n2ccc3c(-c4cccc(-c5ccc(CNC[C@@H]6CCC(=O)N6)c(OC)n5)c4Cl)cccc32)cc(OC)c1CN1CC(C)(C(=O)O)C1]

A mixture of Intermediate E1 (0.12 g, 0.17 mmol) and 3-methylazetidine-3-carboxylic acid (0.02 g, 0.20 mmol) in 4 mL MeOH/DCM (1:1) was stirred at room temperature and acetic acid was added until the pH was around 4. After the reaction was stirred for 1 hour, sodium cyanoborohydride (0.03 g, 0.51 mmol) was added and the reaction was stirred at room temperature for an additional 1 hour. After evaporation to dryness, the resulting crude mixture was stirred at room temperature in the presence of 4M HCl in dioxane for 30 min. All solvent was evaporated under vacuum and the crude mixture was purified by column chromatography (silica gel, gradient elution, 5 to 20% MeOH/DCM). The fractions containing the product were collected and the solvent was removed under reduced pressure, resulting in the title compound. LCMS Method A, RT=2.664 min, m/z=724.3 [M+H]⁺, LCMS Method D, RT=3.645 min, m/z=724.2957 [M+H]⁺, exact mass: 723.2824.

Example 41: (S)-1-(4-(4-(2-chloro-3-(6-methoxy-5-((((5-oxopyrrolidin-2-yl)methyl)amino)methyl)pyridin-2-yl)phenyl)-1H-pyrrolo[2,3-b]pyridin-1-yl)-2,6-dimethoxybenzyl)-3-methylazetidine-3-carboxylic acid

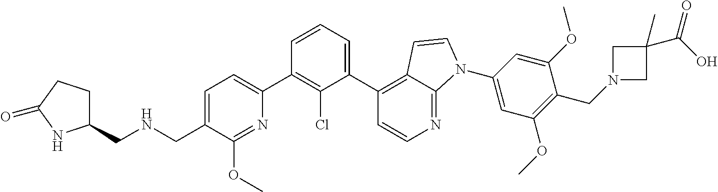 [SMILES: COc1cc(-n2ccc3c(-c4cccc(-c5ccc(CNC[C@@H]6CCC(=O)N6)c(OC)n5)c4Cl)ccnc32)cc(OC)c1CN1CC(C)(C(=O)O)C1]

A mixture of Intermediate E2 (0.12 g, 0.17 mmol) and 3-methylazetidine-3-carboxylic acid (0.02 g, 0.20 mmol) in 4 mL MeOH/DCM (1:1) was stirred at room temperature and acetic acid was added until the pH was around 4. After the reaction was stirred for 1 hour, sodium cyanoborohydride (0.03 g, 0.51 mmol) was added and the reaction was stirred at room temperature for an additional 1 hour. After evaporation to dryness, the resulting crude mixture was stirred at room temperature in the presence of 4M HCl in dioxane for 30 min. All solvent was evaporated under vacuum and the crude mixture was purified by column chromatography (silica gel, gradient elution, 5 to 25% MeOH/DCM). The fractions containing the product were collected and the solvent was removed under reduced pressure, resulting in the title compound. LCMS Method A, RT=2.537 min, m/z=725.3 [M+H]⁺, LCMS Method D, RT=3.566 min, m/z=725.2863 [M+H]⁺, exact mass: 724.2800.

### Example 42: (S)-3-((4-(4-(2-chloro-3-(6-methoxy-5-((((5-oxopyrrolidin-2-yl)methyl)amino)methyl)pyridin-2-yl)phenyl)-1H-indol-1-yl)-2,6-dimethoxybenzyl)amino)-2,2-dimethylpropanoic acid

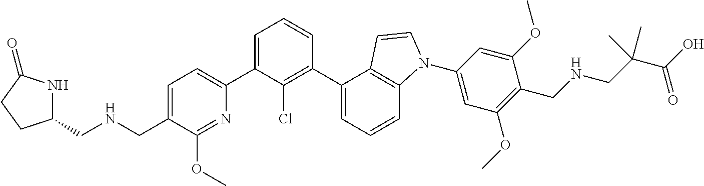 [SMILES: COc1cc(-n2ccc3c(-c4cccc(-c5ccc(CNC[C@@H]6CCC(=O)N6)c(OC)n5)c4Cl)cccc32)cc(OC)c1CNCC(C)(C)C(=O)O]

A mixture of Intermediate E1 (50 mg, 0.07 mmol) and 3-amino-2,2-dimethylpropanoic acid (9.37 mg, 0.08 mmol) in 6 mL MeOH/DCM (1:1) was stirred at room temperature and acetic acid was added until the pH was around 4. After the reaction was stirred for 1 hour, sodium cyanoborohydride (21.99 mg, 0.35 mmol) was added and the reaction was stirred at room temperature for an additional 1 hour. The crude mixture was further used without purification.

The residue was added with 4M HCl in dioxane (1 mL) and stirred at room temperature for 30 minutes. The residual solvent was removed under reduced pressure and the residue was washed with dioxane. The crude mixture was purified by Sephadex column chromatography (100% MeOH), affording the title compound. LCMS method A, RT=2.628 min, m/z=726.3 [M+H]⁺, LCMS Method D, RT=3.800 min, m/z=726.3046 [M+H]⁺, exact mass: 725.2980.

### Example 43: (S)-1-(4-(4-(2-chloro-3-(6-methoxy-5-((((5-oxopyrrolidin-2-yl)methyl)amino)methyl)pyridin-2-yl)phenyl)-1H-indol-1-yl)-2,6-dimethoxybenzyl)-N-cyclopropyl-3-methylazetidine-3-carboxamide

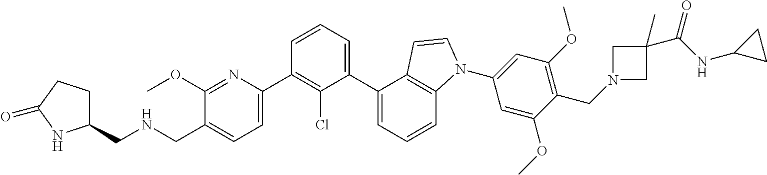 [SMILES: COc1cc(-n2ccc3c(-c4cccc(-c5ccc(CNC[C@@H]6CCC(=O)N6)c(OC)n5)c4Cl)cccc32)cc(OC)c1CN1CC(C)(C(=O)NC2CC2)C1]

### Step 1: (S)-J-(4-(4-(2-chloro-3-(6-methoxy-5-((((5-oxopyrrolidin-2-yl)methyl)amino)methyl)pyridin-2-yl)phenyl)-1H-indol-1-yl)-2,6-dimethoxybenzyl)-3-methylazetidine-3-carboxylicacid

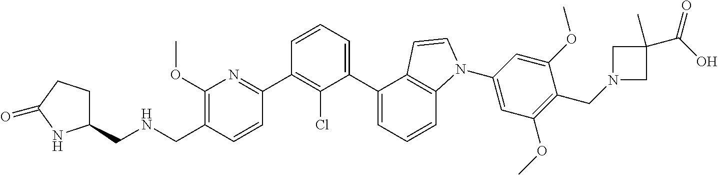 [SMILES: COc1cc(-n2ccc3c(-c4cccc(-c5ccc(CNC[C@@H]6CCC(=O)N6)c(OC)n5)c4Cl)cccc32)cc(OC)c1CN1CC(C)(C(=O)O)C1]

A mixture of Intermediate E1 (100 mg, 0.14 mmol) and 3-methylazetidine-3-carboxylic acid hydrochloride (41.7 mg, 0.27 mmol) in DMF was stirred at room temperature and acetic acid was added until the pH was around 4. After the reaction was stirred for 1 hour, sodium cyanoborohydride (43.36 mg, 0.69 mmol) was added. The reaction was stirred at room temperature for an additional 1 hour, quenched by a small amount of DCM and evaporated to dryness. Then, the residue was dissolved in dioxane and 4M HCl (1 mL) was added. After the reaction was stirred at room temperature for 30 minutes, the residual solvent was removed under reduced pressure and the residue was washed with dioxane. The crude mixture was purified by Sephadex column chromatography (100% MeOH) to provide (S)-1-(4-(4-(2-chloro-3-(6-methoxy-5-((((5-oxopyrrolidin-2-yl)methyl)amino)methyl)pyridin-2-yl)phenyl)-1H-indol-1-yl)-2,6-dimethoxybenzyl)-3-methylazetidine-3-carboxylic acid.

### Step 2: (S)-1-(4-(4-(2-chloro-3-(6-methoxy-5-((((5-oxopyrrolidin-2-yl)methyl)amino)methyl)pyridin-2-yl)phenyl)-1H-indol-1-yl)-2,6-dimethoxybenzyl)-N-cyclopropyl-3-methylazetidine-3-carboxamide

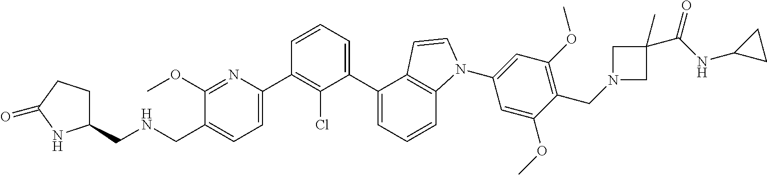 [SMILES: COc1cc(-n2ccc3c(-c4cccc(-c5ccc(CNC[C@@H]6CCC(=O)N6)c(OC)n5)c4Cl)cccc32)cc(OC)c1CN1CC(C)(C(=O)NC2CC2)C1]

A mixture of(S)-1-(4-(4-(2-chloro-3-(6-methoxy-5-((((5-oxopyrrolidin-2-yl)methyl)amino) methyl)pyridin-2-yl)phenyl)-1H-indol-1-yl)-2,6-dimethoxybenzyl)-3-methylazetidine-3-carboxylic acid (100 mg, 0.12 mmol), cyclopropylamine (12.6 μL, 0.18 mmol), 0-(1H-6-Chlorobenzotriazole-1-yl)-1,1,3,3-tetramethyluronium hexafluorophosphate (HCTU) (99.29 mg, 0.24 mmol) and N,N-Diisopropylethylamine (104.51 μL, 0.60 mmol) in DMF (4 mL) was stirred at room temperature for 3 hours. The resulting mixture was extracted 3 times with EtOAc and the organic layer was collected, dried over anhydrous Na₂SO₄, filtered, and evaporated to dryness. The crude mixture was purified by column chromatography (silica gel, gradient elution, 0 to 30% MeOH/DCM) to provide the title compound. LCMS method A, RT=2.619 min, m/z=763.3 [M+H]⁺, LCMS Method D, RT=3.742 min, m/z=763.3361 [M+H]⁺, exact mass: 762.3300.

### Example 44: (S)-1-(((4-(4-(2-chloro-3-(6-methoxy-5-((((5-oxopyrrolidin-2-yl)methyl)amino)methyl)pyridin-2-yl)phenyl)-1H-indol-1-yl)-2,6-dimethoxybenzyl)amino)methyl)cyclopropane-1-carboxylic acid

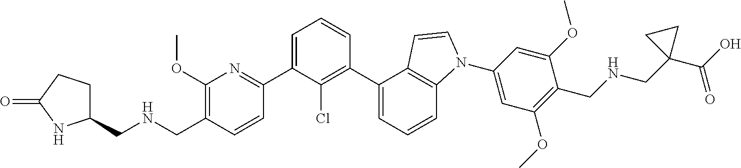 [SMILES: COc1cc(-n2ccc3c(-c4cccc(-c5ccc(CNC[C@@H]6CCC(=O)N6)c(OC)n5)c4Cl)cccc32)cc(OC)c1CNCC1(C(=O)O)CC1]

A mixture of Intermediate E1 (86 mg, 0.12 mmol) and 1-(aminomethyl)cyclopropane-1-carboxylic acid (26.97 mg, 0.18 mmol) in DMF was stirred at room temperature and acetic acid was added until the pH was around 4. After the reaction was stirred for 1 hour, sodium cyanoborohydride (37.70 mg, 0.60 mmol) was added. The reaction was stirred at room temperature for an additional 1 hour, quenched by a small amount of DCM and evaporated to dryness. Then, the residue was dissolved in dioxane and 4M HCl (1 mL) was added. After the reaction was stirred at room temperature for 30 minutes, the residual solvent was removed under reduced pressure and the residue was washed with dioxane. The crude mixture was purified by column chromatography (silica gel, gradient elution, 10% to 50% MeOH/EtOAc) to provide the title compound. LCMS method A, RT=2.69 min, m/z=724.3 [M+H]⁺, LCMS Method D, RT=3.762 min, m/z=724.2914 [M+H]⁺, exact mass: 723.2824.

Example 45: (S)-1-(((4-(4-(2-chloro-3-(6-methoxy-5-((((5-oxopyrrolidin-2-yl)methyl)amino)methyl)pyridin-2-yl)phenyl)-1H-pyrrolo[2,3-b]pyridin-1-yl)-2,6-dimethoxybenzyl)amino)methyl)cyclopropane-1-carboxylic acid

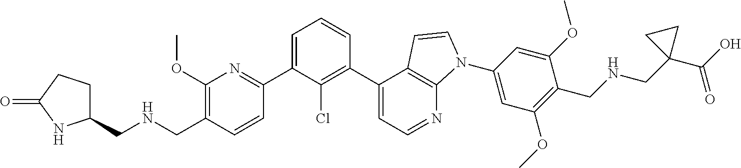 [SMILES: COc1cc(-n2ccc3c(-c4cccc(-c5ccc(CNC[C@@H]6CCC(=O)N6)c(OC)n5)c4Cl)ccnc32)cc(OC)c1CNCC1(C(=O)O)CC1]

A mixture of Intermediate E2 (120 mg, 0.16 mmol) and 1-(aminomethyl)cyclopropane-1-carboxylic acid (30.06 mg, 0.19 mmol) in DMF was stirred at room temperature and acetic acid was added until the pH was around 4. After the reaction was stirred for 1 hour, sodium cyanoborohydride (51.84 mg, 0.82 mmol) was added. The reaction was stirred at room temperature for an additional 1 hour, quenched by a small amount of DCM and evaporated to dryness. Then, the residue was dissolved in dioxane and 4M HCl (1 mL) was added. After the reaction was stirred at room temperature for 30 minutes, the residual solvent was removed under reduced pressure and the residue was washed with dioxane. The crude mixture was purified by column chromatography (silica gel, gradient elution, 10% to 50% MeOH/EtOAc) to provide the title compound. LCMS method B, RT=3.192 min, m/z=725.2 [M+H]⁺, LCMS Method D, RT=3.569 min, m/z=725.2857 [M+H]⁺, exact mass: 724.2800.

Example 46: (S)-1-(4-(4-(2-chloro-3-(6-methoxy-5-((((5-oxopyrrolidin-2-yl)methyl)amino)methyl)pyridin-2-yl)phenyl)-1H-pyrrolo[2,3-b]pyridin-1-yl)-2,6-dimethoxybenzyl)-N-cyclopropyl-3-methylazetidine-3-carboxamide

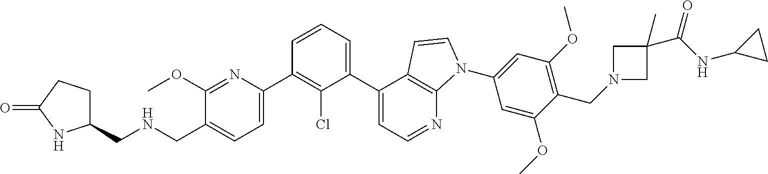 [SMILES: COc1cc(-n2ccc3c(-c4cccc(-c5ccc(CNC[C@@H]6CCC(=O)N6)c(OC)n5)c4Cl)ccnc32)cc(OC)c1CN1CC(C)(C(=O)NC2CC2)C1]

### Step 1: (S)-1-(4-(4-(2-chloro-3-(6-methoxy-5-((((5-oxopyrrolidin-2-yl)methyl)amino)methyl)pyridin-2-yl)phenyl)-1H-pyrrolo[2,3-b]pyridin-1-yl)-2,6-dimethoxybenzyl)-3-methylazetidine-3-carboxylic acid

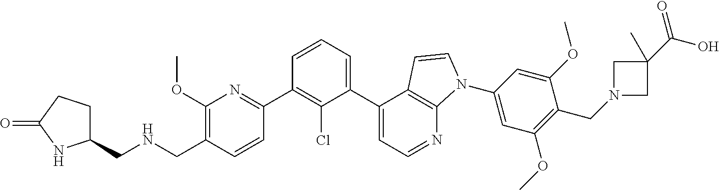 [SMILES: COc1cc(-n2ccc3c(-c4cccc(-c5ccc(CNC[C@@H]6CCC(=O)N6)c(OC)n5)c4Cl)ccnc32)cc(OC)c1CN1CC(C)(C(=O)O)C1]

A mixture of Intermediate E2 (130 mg, 0.18 mmol) and 3-methylazetidine-3-carboxylic acid hydrochloride (54.13 mg, 0.36 mmol) in DMF was stirred at room temperature and acetic acid was added until the pH was around 4. After the reaction was stirred for 1 hour, sodium cyanoborohydride (56.60 mg, 0.90 mmol) was added. The reaction was stirred at room temperature for an additional 1 hour, quenched by a small amount of DCM and evaporated to dryness. Then, the residue was dissolved in dioxane and 4M HCl (1 mL) was added. After the reaction was stirred at room temperature for 30 minutes, the residual solvent was removed under reduced pressure and the residue was washed with dioxane. The crude mixture was purified by Sephadex column chromatography (100% MeOH) to provide (S)-1-(4-(4-(2-chloro-3-(6-methoxy-5-((((5-oxopyrrolidin-2-yl)methyl)amino)methyl)pyridin-2-yl)phenyl)-1H-pyrrolo[2,3-b]pyridin-1-yl)-2,6-dimethoxybenzyl)-3-methylazetidine-3-carboxylic acid.

Step 2: (S)-1-(4-(4-(2-chloro-3-(6-methoxy-5-((((5-oxopyrrolidin-2-yl)methyl)amino)methyl)pyridin-2-yl)phenyl)-1H-pyrrolo[2,3-b]pyridin-1-yl)-2,6-dimethoxybenzyl)-N-cyclopropyl-3-methylazetidine-3-carboxamide

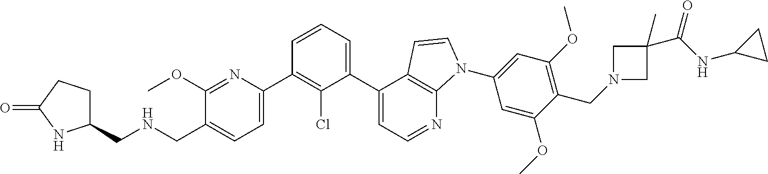 [SMILES: COc1cc(-n2ccc3c(-c4cccc(-c5ccc(CNC[C@@H]6CCC(=O)N6)c(OC)n5)c4Cl)ccnc32)cc(OC)c1CN1CC(C)(C(=O)NC2CC2)C1]

A mixture of (S)-1-(4-(4-(2-chloro-3-(6-methoxy-5-((((5-oxopyrrolidin-2-yl)methyl)amino)methyl)pyridin-2-yl)phenyl)-1H-pyrrolo[2,3-b]pyridin-1-yl)-2,6-dimethoxybenzyl)-3-methylazetidine-3-carboxylic acid (40 mg, 0.05 mmol), cyclopropylamine (5.74 μL, 0.08 mmol), 0-(1H-6-Chlorobenzotriazole-1-yl)-1,1,3,3-tetramethyluronium hexafluorophosphate (HCTU) (45.50 mg, 0.11 mmol) and N,N-Diisopropylethylamine (47.98 μL, 0.27 mmol) in DMF (4 mL) was stirred at room temperature for 3 hours. The resulting mixture was extracted 3 times with EtOAc and the organic layer was collected, dried over anhydrous Na₂SO₄, filtered, and evaporated to dryness. The crude mixture was purified by column chromatography (silica gel, gradient elution, 0 to 30% MeOH/DCM) to provide the title compound. LCMS method B, RT=3.153 min, m/z=764.3 [M+H]⁺, LCMS Method D, RT=3.602 min, m/z=764.3314 [M+H]⁺, exact mass: 763.3200.

### Example 47: 1-(4-(4-(2-chloro-3-(5-((((cis-3-hydroxycyclobutyl)methyl)amino)methyl)-6-methoxypyridin-2-yl)phenyl)-1H-indazol-1-yl)-2,6-dimethoxybenzyl)-3-methylazetidine-3-carboxylic acid

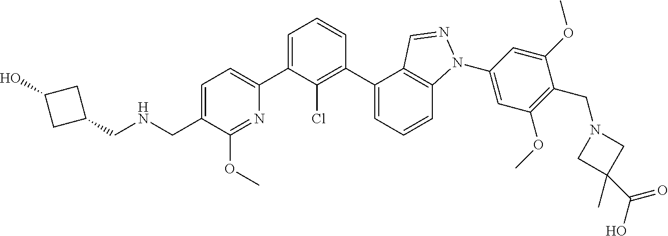 [SMILES: COc1cc(-n2ncc3c(-c4cccc(-c5ccc(CNC[C@H]6C[C@@H](O)C6)c(OC)n5)c4Cl)cccc32)cc(OC)c1CN1CC(C)(C(=O)O)C1]

A mixture of Intermediate E4 (50 mg, 0.07 mmol) and 3-methylazetidine-3-carboxylic acid hydrochloride (12.12 mg, 0.08 mmol) in 6 mL MeOH/DCM (1:1) was stirred at room temperature and acetic acid was added until the pH was around 4. After the reaction was stirred for 1 hour, sodium cyanoborohydride (21.99 mg, 0.35 mmol) was added and the reaction was stirred at room temperature for an additional 1 hour. The crude mixture was further used without purification.

The residue was added with 4M HCl in dioxane (1 mL) and stirred at room temperature for 30 minutes. The residual solvent was removed under reduced pressure and the residue was washed with dioxane. The crude mixture was purified by Sephadex column chromatography (100% MeOH), affording the title compound. LCMS method B, RT=3.135 min, m/z=712.3 [M+H]⁺, LCMS Method D, RT=3.689 min, m/z=712.2878 [M+H]⁺, exact mass: 711.2824.

### Example 48: (S)-3-(((6-(2-chloro-3-(1-(3,5-dimethoxy-4-((((5-oxopyrrolidin-2-yl)methyl)amino)methyl)phenyl)-1H-indol-4-yl)phenyl)-2-methoxypyridin-3-yl)methyl)amino)-2,2-dimethylpropanoic acid

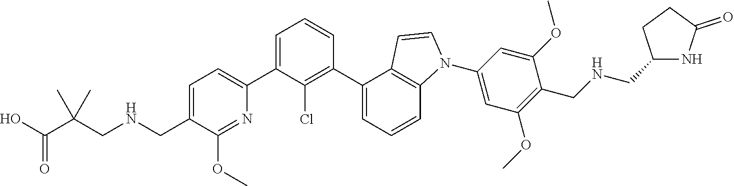 [SMILES: COc1cc(-n2ccc3c(-c4cccc(-c5ccc(CNCC(C)(C)C(=O)O)c(OC)n5)c4Cl)cccc32)cc(OC)c1CNC[C@@H]1CCC(=O)N1]

A mixture of Intermediate F1 (50 mg, 0.07 mmol) and 3-amino-2,2-dimethylpropanoic acid (9.37 mg, 0.08 mmol) in 6 mL MeOH/DCM (1:1) was stirred at room temperature and acetic acid was added until the pH was around 4. After the reaction was stirred for 1 hour, sodium cyanoborohydride (21.99 mg, 0.35 mmol) was added and the reaction was stirred at room temperature for an additional 1 hour. The crude mixture was further used without purification.

The residue was added with 4M HCl in dioxane (1 mL) and stirred at room temperature for 30 minutes. The residual solvent was removed under reduced pressure and the residue was washed with dioxane. The crude mixture was purified by Sephadex column chromatography (100% MeOH), affording the title compound. LCMS Method B, RT=3.960 min, m/z=726.3 [M+H]⁺, LCMS Method D, RT=3.540 min, m/z=726.3052 [M+H]⁺, exact mass: 725.2980.

Example 49: (3S)-3-(((6-(2-chloro-3-(1-(3,5-dimethoxy-4-(((((S)-5-oxopyrrolidin-2-yl)methyl)amino)methyl)phenyl)-1H-indol-4-yl)phenyl)-2-methoxypyridin-3-yl)methyl)amino)cyclopentane-1-carboxylic acid

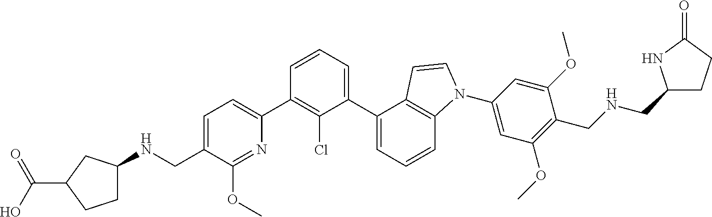 [SMILES: COc1cc(-n2ccc3c(-c4cccc(-c5ccc(CN[C@H]6CCC(C(=O)O)C6)c(OC)n5)c4Cl)cccc32)cc(OC)c1CNC[C@@H]1CCC(=O)N1]

A mixture of Intermediate F1 (60 mg, 0.08 mmol) and (3S)-3-aminocyclopentane-1-carboxylic acid hydrochloride (11.76 mg, 0.09 mmol) in DMF was stirred at room temperature and acetic acid was added until the pH was around 4. After the reaction was stirred for 1 hour, sodium cyanoborohydride (25.13 mg, 0.4 mmol) was added. The reaction was stirred at room temperature for an additional 1 hour, quenched by a small amount of DCM and evaporated to dryness. Then, the residue was dissolved in dioxane and 4M HCl (1 mL) was added. After the reaction was stirred at room temperature for 30 minutes, the residual solvent was removed under reduced pressure and the residue was washed with dioxane. The reaction mixture was purified by Sephadex column chromatography (100% MeOH) to provide the title compound. LCMS method A, RT=2.63 min, m/z=738.4 [M+H]⁺, LCMS Method D, RT=3.749 min, m/z=738.3057 [M+H]⁺, exact mass: 737.2980.

Example 50: Methyl (3S)-3-(((6-(2-chloro-3-(1-(3,5-dimethoxy-4-(((((S)-5-oxopyrrolidin-2-yl)methyl)amino)methyl)phenyl)-1H-indol-4-yl)phenyl)-2-methoxypyridin-3-yl)methyl)amino)cyclopentane-1-carboxylate

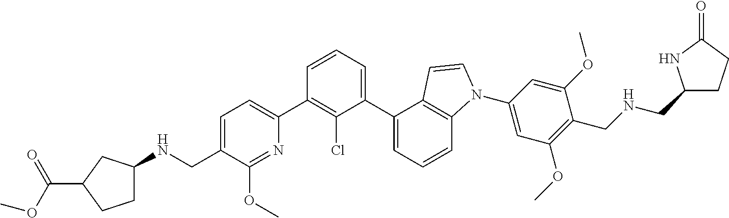 [SMILES: COC(=O)C1CC[C@H](NCc2ccc(-c3cccc(-c4cccc5c4ccn5-c4cc(OC)c(CNC[C@@H]5CCC(=O)N5)c(OC)c4)c3Cl)nc2OC)C1]

A mixture of Example 49 (30 mg, 0.04 mmol) and 4M HCl in dioxane (1 mL) in MeOH was stirred at room temperature for 30 minutes. The residual solvent was removed under reduced pressure and the residue was washed with dioxane. The crude mixture was purified by 0-10% MeOH/DCM, resulting in the title compound. LCMS method C, RT=4.159 min, m/z=752.3 [M+H]⁺, LCMS Method D, RT=3.870 min, m/z=752.3193 [M+H]⁺, exact mass: 751.3137.

### Example 51: (S)-5-((((6-(2-chloro-3-(1-(4-((3-hydroxy-3-methylazetidin-1-yl)methyl)-3,5-dimethoxyphenyl)-1H-indazol-4-yl)phenyl)-2-methoxypyridin-3-yl)methyl)amino)methyl)pyrrolidin-2-one

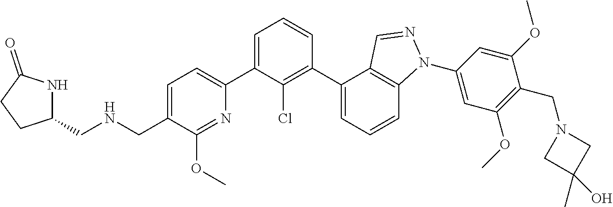 [SMILES: COc1cc(-n2ncc3c(-c4cccc(-c5ccc(CNC[C@@H]6CCC(=O)N6)c(OC)n5)c4Cl)cccc32)cc(OC)c1CN1CC(C)(O)C1]

A mixture of Intermediate E3 (50 mg, 0.07 mmol) and 3-methylazetidin-3-ol hydrochloride (9.88 mg, 0.08 mmol) in 6 mL MeOH/DCM (1:1) was stirred at room temperature and acetic acid was added until the pH was around 4. After the reaction was stirred for 1 hour, sodium cyanoborohydride (21.99 mg, 0.35 mmol) was added and the reaction was stirred at room temperature for an additional 1 hour. The crude mixture was further used without purification.

The residue was added with 4M HCl in dioxane (1 mL) and stirred at room temperature for 30 minutes. The residual solvent was removed under reduced pressure and the residue was washed with dioxane. The crude mixture was purified by Sephadex column chromatography (100% MeOH), affording the title compound. LCMS method B, RT=3.102 min, m/z=697.3 [M+H]⁺, LCMS Method D, RT=3.473 min, m/z=697.2949 [M+H]⁺, exact mass: 696.2827.

### Example 52: (S)-1-(4-(4-(2-chloro-3-(6-methoxy-5-((((5-oxopyrrolidin-2-yl)methyl)amino)methyl)pyridin-2-yl)phenyl)-1H-indazol-1-γ)-2,6-dimethoxybenzyl)-3-methylazetidine-3-carboxylic acid

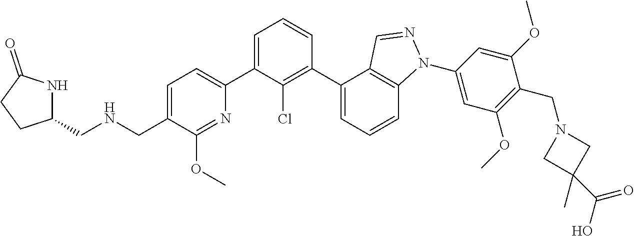 [SMILES: COc1cc(-n2ncc3c(-c4cccc(-c5ccc(CNC[C@@H]6CCC(=O)N6)c(OC)n5)c4Cl)cccc32)cc(OC)c1CN1CC(C)(C(=O)O)C1]

A mixture of Intermediate E3 (50 mg, 0.07 mmol) and 3-methylazetidine-3-carboxylic acid hydrochloride (12.12 mg, 0.08 mmol) in 6 mL MeOH/DCM (1:1) was stirred at room temperature and acetic acid was added until the pH was around 4. After the reaction was stirred for 1 hour, sodium cyanoborohydride (21.99 mg, 0.35 mmol) was added and the reaction was stirred at room temperature for an additional 1 hour. The crude mixture was further used without purification.

The residue was added with 4M HCl in dioxane (1 mL) and stirred at room temperature for 30 minutes. The residual solvent was removed under reduced pressure and the residue was washed with dioxane. The crude mixture was purified by Sephadex column chromatography (100% MeOH), affording the title compound. LCMS method B, RT=3.123 min, m/z=725.3 [M+H]⁺, LCMS Method D, RT=3.566 min, m/z=725.2898 [M+H]⁺, exact mass: 724.2776. NMR (500 MHz, MeOD-d₄) δ 8.05 (d, J=2.7 Hz, 1H), 7.95 (d, J=7.8 Hz, 1H), 7.75 (d, J=6.5 Hz, 1H), 7.69-7.60 (m, 3H), 7.58-7.50 (m, 3H), 7.33-7.25 (m, 2H), 7.14 (s, 1H), 4.46 (s, 2H), 4.39 (s, 2H), 4.03 (s, 3H), 4.01 (s, 3H), 3.91 (d, J=10.8 Hz, 2H), 3.85 (d, J=5.9 Hz, 2H), 2.43-2.24 (m, 6H), 1.53 (d, J=8.7 Hz, 3H).

### Example 53: (S)-5-((((6-(2-chloro-3-(1-(4-((3-hydroxy-3-methylazetidin-1-yl)methyl)-3,5-dimethoxyphenyl)-1H-indol-4-yl)phenyl)-2-methoxypyridin-3-yl)methyl)amino)methyl)pyrrolidin-2-one

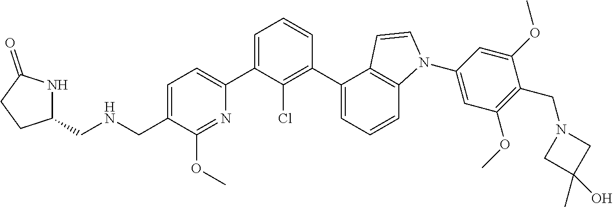 [SMILES: COc1cc(-n2ccc3c(-c4cccc(-c5ccc(CNC[C@@H]6CCC(=O)N6)c(OC)n5)c4Cl)cccc32)cc(OC)c1CN1CC(C)(O)C1]

A mixture of Intermediate E1 (50 mg, 0.07 mmol) and 3-methylazetidin-3-ol hydrochloride (9.88 mg, 0.08 mmol) in 6 mL MeOH/DCM (1:1) was stirred at room temperature and acetic acid was added until the pH was around 4. After the reaction was stirred for 1 hour, sodium cyanoborohydride (21.99 mg, 0.35 mmol) was added and the reaction was stirred at room temperature for an additional 1 hour. The crude mixture was further used without purification.

The residue was added with 4M HCl in dioxane (1 mL) and stirred at room temperature for 30 minutes. The residual solvent was removed under reduced pressure and the residue was washed with dioxane. The crude mixture was purified by Sephadex column chromatography (100% MeOH), affording the title compound. LCMS method B, RT=3.163 min, m/z=696.3 [M+H]⁺, LCMS Method D, RT=3.691 min, m/z=696.2949 [M+H]⁺, exact mass: 695.2874.

### Example 54: (S)-5-(((4-(4-(2-chloro-3-(5-((3-hydroxy-3-methylazetidin-1-yl)methyl)-6-methoxypyridin-2-yl)phenyl)-1H-indazol-1-yl)-2,6-dimethoxybenzyl)amino)methyl)pyrrolidin-2-one

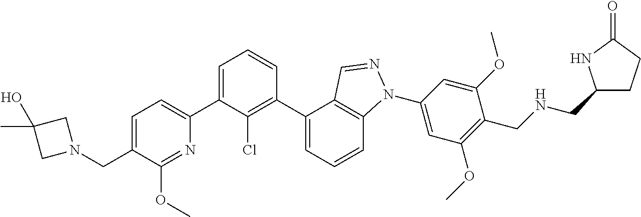 [SMILES: COc1cc(-n2ncc3c(-c4cccc(-c5ccc(CN6CC(C)(O)C6)c(OC)n5)c4Cl)cccc32)cc(OC)c1CNC[C@@H]1CCC(=O)N1]

A mixture of Intermediate F2 (0.12 g, 0.17 mmol) and 3-methylazetidin-3-ol (0.02 g, 0.20 mmol) in 4 mL MeOH/DCM (1:1) was stirred at room temperature and acetic acid was added until the pH was around 4. After the reaction was stirred for 1 hour, sodium cyanoborohydride (0.03 g, 0.51 mmol) was added and the reaction was stirred at room temperature for an additional 1 hour. After evaporation to dryness, the resulting mixture was stirred at room temperature in the presence of 4M HCl in dioxane for 30 min. The solvent was evaporated under vacuum and the crude mixture was purified by column chromatography (silica gel, gradient elution, 5 to 25% MeOH/DCM). The fractions containing the product were collected and the solvent was removed under reduced pressure, resulting in the title compound. LCMS Method B, RT=3.0470 min, m/z=697.3 [M+H]⁺, LCMS Method D, RT=3.593 min, m/z=697.2895 [M+H]⁺, exact mass: 696.2800.

### Example 55: (S)-5-(((4-(4-(2-chloro-3-(5-((((cis-3-hydroxycyclobutyl)methyl)amino)methyl)-6-methoxypyridin-2-yl)phenyl)-1H-indazol-1-yl)-2,6-dimethoxybenzyl)amino)methyl)pyrrolidin-2-one

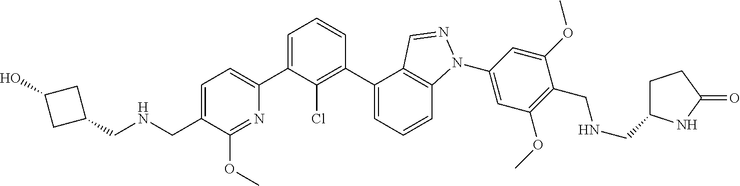 [SMILES: COc1cc(-n2ncc3c(-c4cccc(-c5ccc(CNC[C@H]6C[C@@H](O)C6)c(OC)n5)c4Cl)cccc32)cc(OC)c1CNC[C@@H]1CCC(=O)N1]

A mixture of Intermediate E4 (50 mg, 0.07 mmol) and (S)-5-(aminomethyl)pyrrolidin-2-one (9.13 mg, 0.08 mmol) in 6 mL MeOH/DCM (1:1) was stirred at room temperature and acetic acid was added until the pH around 4. After leaving for 1 hour, sodium cyanoborohydride (21.99 mg, 0.35 mmol) was added and the reaction was stirred at room temperature for an additional 1 hour before 4M HCl in dioxane (1 mL) was added. After 30 minutes, the solvent was removed under reduced pressure and the residue was washed with dioxane. The crude mixture was purified by Sephadex column chromatography (100% MeOH), affording the title compound. LCMS method B, RT=3.055 min, m/z=711.4 [M+H]⁺, LCMS Method D, RT=3.544 min, m/z=711.3062 [M+H]⁺, exact mass: 710.2983.

### Example 56: 2-((4-((3-(1-(4-(((2-carboxy-1-hydroxypropan-2-yl)amino)methyl)-3,5-dimethoxyphenyl)-1H-indol-4-yl)-2-methylphenyl)carbamoyl)benzyl)amino)-3-hydroxy-2-methylpropanoic acid

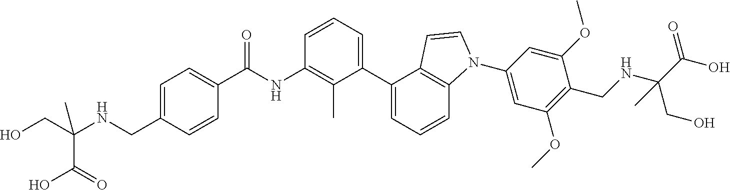 [SMILES: COc1cc(-n2ccc3c(-c4cccc(NC(=O)c5ccc(CNC(C)(CO)C(=O)O)cc5)c4C)cccc32)cc(OC)c1CNC(C)(CO)C(=O)O]

### Step 1: methyl 4-(dimethoxymethyl)benzoate

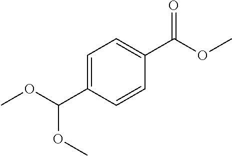

A mixture of methyl 4-formylbenzoate (3.01 g, 18.3 mmol), trimethyl orthoformate (3.0 mL, 27.4 mmol) and pyridinium p-toluenesulfonate (0.15 g, 0.60 mmol) in MeOH 75 mL was stirred at refluxing temperature for 4.5 hours. Then, the mixture was cooled down and diluted with sat·NaHCO₃. The resulting mixture was extracted 3 times with EtOAc and the organic layer was collected, dried over anhydrous Na₂SO₄, filtered, and evaporated to dryness. The crude mixture was purified by column chromatography (silica gel, gradient elution, 50 to 100% DCM/hexanes) to provide methyl 4-(dimethoxymethyl)benzoate (3.47 g). LCMS Method C, RT=4.443 min, m/z=179.1 [M-OMe]⁺, exact mass: 210.0892.

### Step 2: N-(3-bromo-2-methylphenyl)-4-formylbenzamide

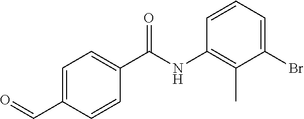 [SMILES: Cc1c(Br)cccc1NC(=O)c1ccc(C=O)cc1]

Potassium tert-butoxide (4.0 mL, 1M in THF) was added to a mixture of methyl 4-(dimethoxymethyl)benzoate (0.92 g, 4.38 mmol) and 3-bromo-2-methylaniline (0.68 g, 3.65 mmol) in THF 10 mL at rt under argon atmosphere. Then, the mixture was stirred for 6.5 hours and was quenched with water. The resulting mixture was extracted 3 times with EtOAc and the organic layer was collected, dried over anhydrous Na₂SO₄, filtered, and evaporated to dryness.

The crude residue was dissolved in DCM 10 mL before TFA (0.28 mL, 3.66 mmol) was added to this solution at rt. After the reaction was stirred for 4 hours, evaporated to dryness, water was transferred to this residue. The resulting mixture was extracted 3 times with DCM and the organic layer was collected, dried over anhydrous Na₂SO₄, filtered, and evaporated to dryness. The crude mixture was purified by precipitation in DCM/hexanes to provide N-(3-bromo-2-methylphenyl)-4-formylbenzamide (0.74 g). LCMS Method C, RT=4.483 min, m/z=318.0 [M+H]⁺, exact mass: 317.0051.

### Step 3: 4-formyl-N-(3-(1-(4-formyl-3,5-dimethoxyphenyl)-1H-indol-4-yl)-2-methylphenyl)benzamide

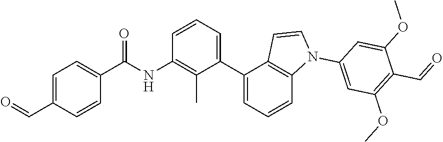 [SMILES: COc1cc(-n2ccc3c(-c4cccc(NC(=O)c5ccc(C=O)cc5)c4C)cccc32)cc(OC)c1C=O]

A mixture of Intermediate B1-1 (0.64 g, 1.57 mmol), N-(3-bromo-2-methylphenyl)-4-formylbenzamide (0.50 g, 1.57 mmol), Pd(PPh₃)₄ (0.18 g, 0.16 mmol) and K₂CO₃ (0.57 g, 4.12 mmol) in a mixture of 1,4-dioxane and H₂O (6:1) 14 mL was sparged with argon for 10 min before stirring at 100° C. for overnight under argon atmosphere. Then, the mixture was cooled down and diluted with brine. The resulting mixture was extracted 3 times with EtOAc and the organic layer was collected, dried over anhydrous Na₂SO₄, filtered, and evaporated to dryness. The crude mixture was purified by column chromatography (silica gel, gradient elution, 0 to 100% EtOAc/hexanes) to provide 4-formyl-N-(3-(I-(4-formyl-3,5-dimethoxyphenyl)-1H-indol-4-yl)-2-methylphenyl)benzamide (0.56 g). LCMS Method C, RT=4.882 min, m/z=519.2 [M+H]⁺, exact mass: 518.1842.

### Step 4: 2-((4-((3-(1-(4-(((2-carboxy-1-hydroxypropan-2-yl)amino)methyl)-3,5-dimethoxyphenyl)-1H-indol-4-yl)-2-methylphenyl)carbamoyl)benzyl)amino)-3-hydroxy-2-methylpropanoic acid

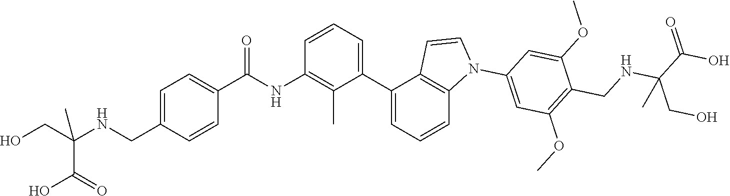 [SMILES: COc1cc(-n2ccc3c(-c4cccc(NC(=O)c5ccc(CNC(C)(CO)C(=O)O)cc5)c4C)cccc32)cc(OC)c1CNC(C)(CO)C(=O)O]

A mixture of 4-formyl-N-(3-(1-(4-formyl-3,5-dimethoxyphenyl)-1H-indol-4-yl)-2-methylphenyl)benzamide (77.6 mg, 0.15 mmol) and 2-amino-3-hydroxy-2-methylpropanoic acid (79.6 mg, 0.76 mmol) in 6 mL MeOH/DCM (1:1) was stirred at room temperature and acetic acid was added until the pH was around 4. After the reaction was stirred for 1 hour, sodium cyanoborohydride (58.0 mg, 0.92 mmol) was added and the reaction was stirred at room temperature for overnight. The reaction mixture was purified by column chromatography (silica gel, gradient elution, 0 to 100% MeOH/DCM). The fractions containing the product were collected and the solvent was removed under reduced pressure to provide the title compound. LCMS Method C, RT=3.999 min, m/z=723.4 [M+H]⁺, exact mass: 724.3108.

### Example 57: (S)-2-(((4-(2,3-dihydrobenzo[b][1,4]dioxin-6-yl)-1′-methyl-1′H-[1,6′-biindol]-2′-yl)methyl)amino)-3-hydroxypropanoic acid

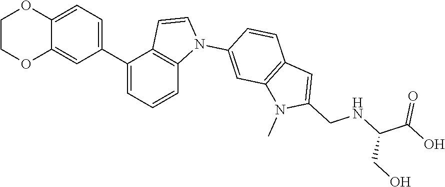 [SMILES: Cn1c(CN[C@@H](CO)C(=O)O)cc2ccc(-n3ccc4c(-c5ccc6c(c5)OCCO6)cccc43)cc21]

### Step 1: methyl 6-bromo-1-methyl-JH-indole-2-carboxylate

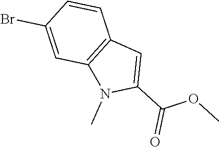

MeI (0.58 mL, 9.32 mmol) was added to a mixture of 6-bromo-1H-indole-2-carboxylic acid (1.00 g, 4.16 mmol) and K₂CO₃ (1.48 g, 10.7 mmol) in DMF 14 mL at rt. After the reaction mixture was stirred at rt for 1 hour, it was stirred at 60° C. for 24 hours. Then another portion of MeI (0.59 mL, 9.48 mmol) was added to the reaction mixture at rt. The resulting mixture was stirred at 60° C. for 24 hours before water was poured to this reaction at rt. The resulting solid was filtered and washed with water. The pink solid was purified by column chromatography (silica gel, gradient elution, 0 to 20% EtOAc/hexanes) to provide methyl 6-bromo-1-methyl-1H-indole-2-carboxylate (256.5 mg). LCMS Method C, RT=5.296 min, m/z=268.0 [M+H]⁺, exact mass: 266.9895.

### Step 2: 6-bromo-1-methyl-1H-indole-2-carbaldehyde

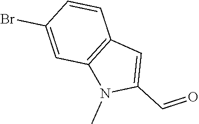

LiAlH₄ (0.34 mL, 2.5M in THF) was added to a solution of methyl 6-bromo-1-methyl-1H-indole-2-carboxylate (0.23 g, 0.86 mmol) in THF 4 mL at 0° C. under argon atmosphere. Then, the mixture was stirred at rt for 30 min and was quenched with sat·NH₄Cl at 0° C. The resulting mixture was extracted 3 times with EtOAc and the organic layer was collected, dried over anhydrous Na₂SO₄, filtered, and evaporated to dryness.

A solution of Dess-Martin periodinane (0.44 g, 1.04 mmol) and pyridine (0.20 mL, 2.47 mmol) in DCM 5 mL was added to a solution of crude yellow solid in DCM 5 mL at rt. After the reaction was stirred for 4 hours, evaporated to dryness, the crude mixture was purified by column chromatography (silica gel, gradient elution, 0 to 30% EtOAc/hexanes) to provide 6-bromo-1-methyl-1H-indole-2-carbaldehyde (147.4 mg). LCMS Method C, RT=4.967 min, m/z=238.0 [M+H]⁺, exact mass. 236.9789.

### Step 3: 4-(2,3-dihydrobenzo[b][1,4]dioxin-6-yl)-1′-methyl-1′H-[1,6′-biindole]-2′-carbaldehyde

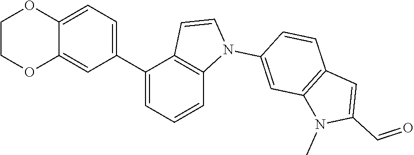 [SMILES: Cn1c(C=O)cc2ccc(-n3ccc4c(-c5ccc6c(c5)OCCO6)cccc43)cc21]

A mixture of Intermediate A1 (0.13 g, 0.52 mmol), 6-bromo-1-methyl-1H-indole-2-carbaldehyde (0.15 g, 0.63 mmol), Pd₂(dba)₃ (0.02 g, 0.02 mmol), Xantphos (0.03 g, 0.05 mmol) and Cs₂CO₃ (0.35 g, 1.07 mmol) in toluene 13 mL was sparged with argon for 10 min before it was refluxed for overnight under argon atmosphere. Then, the mixture was cooled down and diluted with water. The resulting mixture was extracted 3 times with EtOAc and the organic layer was collected, dried over anhydrous Na₂SO₄, filtered, and evaporated to dryness. The crude mixture was purified by column chromatography (silica gel, gradient elution, 0 to 100% EtOAc/hexanes) to provide 4-(2,3-dihydrobenzo[b][1,4]dioxin-6-yl)-1′-methyl-1′H-[1,6′-biindole]-2′-carbaldehyde (0.14 g). LCMS Method C, RT=6.075 min, m/z=409.2 [M+H]⁺, exact mass: 408.1474.

### Step 4: (S)-2-(((4-(2,3-dihydrobenzo[b][1,4]dioxin-6-yl)-1′-methyl-1′H-[1,6′-biindol]-2′-yl)methyl)amino)-3-hydroxypropanoic acid

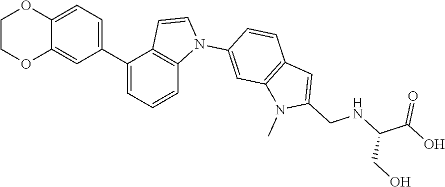 [SMILES: Cn1c(CN[C@@H](CO)C(=O)O)cc2ccc(-n3ccc4c(-c5ccc6c(c5)OCCO6)cccc43)cc21]

A mixture of 4-(2,3-dihydrobenzo[b][1,4]dioxin-6-yl)-1′-methyl-1′H-[1,6′-biindole]-2′-carbaldehyde (60.4 mg, 0.15 mmol) and L-serine (20.8 mg, 0.19 mmol) in 4 mL MeOH/DCM (1:1) was stirred at room temperature and acetic acid was added until the pH was around 4. After 12 hours, sodium cyanoborohydride (44.6 mg, 0.65 mmol) was added and the reaction was stirred at room temperature for 3 hours. The crude mixture was purified by column chromatography (silica gel, gradient elution, 0 to 100% MeOH/DCM). The fractions containing the product were collected and the solvent was removed under reduced pressure to provide the title compound. LCMS Method C, RT=4.406 min, m/z=496.3 [M−H]⁻, LCMS Method D, RT=4.353 min, m/z=498.2001 [M+H]⁺, exact mass: 497.1951.

### Example 58: 1-(6-((6-(4-(2,3-dihydrobenzo[b][1,4]dioxin-6-yl)-1H-indol-1-yl)-2-methoxypyridin-3-yl)methyl)-2,6-diazaspiro[3.3]heptan-2-yl)ethanone

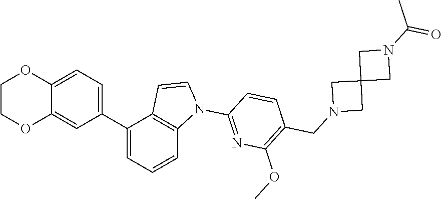 [SMILES: COc1nc(-n2ccc3c(-c4ccc5c(c4)OCCO5)cccc32)ccc1CN1CC2(C1)CN(C(C)=O)C2]

### Step 1: tert-butyl 6-((6-(4-(2,3-dihydrobenzo[b][1,4]dioxin-6-yl)-1H-indol-1-yl)-2-methoxypyridin-3-yl)methyl)-2,6-diazaspiro[3.3]heptane-2-carboxylate

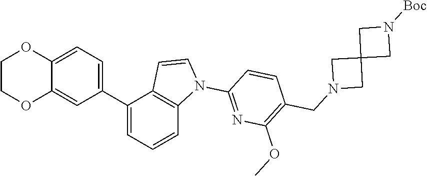 [SMILES: COc1nc(-n2ccc3c(-c4ccc5c(c4)OCCO5)cccc32)ccc1CN1CC2(C1)CN(C(=O)OC(C)(C)C)C2]

A mixture of Intermediate A-4 (77.2 mg, 0.20 mmol) and 2,6-diazaspiro[3.3]heptane-2-carboxylic acid tert-butyl ester hemioxalate (59.4 mg, 0.12 mmol) in 5 mL MeOH/DCM (1:1) was stirred at room temperature and acetic acid was added until the pH was around 4. After the reaction was stirred for 1 hour, sodium cyanoborohydride (56.7 mg, 0.82 mmol) was added and the reaction was stirred at room temperature for overnight. The reaction mixture was purified by column chromatography (silica gel, gradient elution, 0 to 100% MeOH/DCM). The fractions containing the product were collected and the solvent was removed under reduced pressure to provide tert-butyl 6-((6-(4-(2,3-dihydrobenzo[b][1,4]dioxin-6-yl)-1H-indol-1-yl)-2-methoxypyridin-3-yl)methyl)-2,6-diazaspiro[3.3]heptane-2-carboxylate (28.9 mg). LCMS Method C, RT=4.604 min, m/z=569.3 [M+H]⁺, exact mass: 568.2686.

### Step 2: 1-(6-((6-(4-(2,3-dihydrobenzo[b][1,4]dioxin-6-yl)-1H-indol-1-yl)-2-methoxypyridin-3-yl)methyl)-2,6-diazaspiro[3.3]heptan-2-yl)ethanone

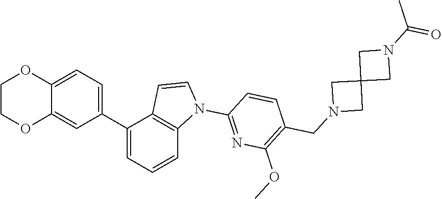 [SMILES: COc1nc(-n2ccc3c(-c4ccc5c(c4)OCCO5)cccc32)ccc1CN1CC2(C1)CN(C(C)=O)C2]

After tert-butyl 6-((6-(4-(2,3-dihydrobenzo[b][1,4]dioxin-6-yl)-1H-indol-1-yl)-2-methoxypyridin-3-yl)methyl)-2,6-diazaspiro[3.3]heptane-2-carboxylate (28.9 mg, 0.05 mmol) was dissolved in TFA/DCM (1:1) 1 mL, the solution was stirred for 1 hour at room temperature. The resulting mixture was quenched with sat·NaHCO₃, extracted 3 times with EtOAc and the organic layer was collected, dried over anhydrous Na₂SO₄, filtered, and evaporated to dryness.

NEt₃ (0.6 mL, 4.30 mmol) and AcOH (0.6 mL, 6.36 mmol) were added to a solution of the crude residue in THF 2 mL before stirring at rt for 7 hours. After the resulting mixture was quenched with water and evaporated to dryness, the mixture was purified by silica gel chromatography (0 to 100% MeOH/DCM). The fractions containing the product were collected and the solvent was removed under reduced pressure to provide the title compound. LCMS Method C, RT=4.385 min, m/z=511.3 [M+H]⁺, LCMS Method D, RT=4.154 min, m/z=511.2301 [M+H]⁺, exact mass: 510.2267.

### Example 59: 2-(4-(2,3-dihydrobenzo[b] [1,4]dioxin-6-yl)-1H-indol-1-yl)-4,5,6,7-tetrahydrothiazolo[5,4-c]pyridine

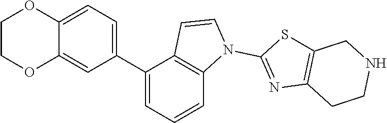 [SMILES: c1cc(-c2ccc3c(c2)OCCO3)c2ccn(-c3nc4c(s3)CNCC4)c2c1]

A mixture of Intermediate A1 (146.4 mg, 0.58 mmol), tert-butyl 2-bromo-6,7-dihydrothiazole[5,4-c]pyridine-5 (4H)-carboxylate (187.4 mg, 0.59 mmol), Pd₂(dba)₃ (17.0 mg, 0.02 mmol), tBuXPhos (28.4 mg, 0.07 mmol) and NaO^{t}Bu (196.4 mg, 2.04 mmol) in toluene 9 mL was sparged with argon for 10 min. Then, the mixture was refluxed overnight under argon atmosphere. The mixture was cooled down, filtered, and evaporated to dryness. The crude mixture was purified by column chromatography (silica gel, gradient elution, 0 to 100% EtOAc/hexanes) to provide tert-butyl 2-(4-(2,3-dihydrobenzo[b][1,4]dioxin-6-yl)-1H-indol-1-yl)-6,7-dihydrothiazolo[5,4-c]pyridine-5 (4H)-carboxylate (20.6 mg). LCMS Method C, RT=5.690 min, m/z=490.2 [M+H]⁺, exact mass: 489.1722.

Then, the compound was dissolved in TFA/DCM (1:1) 1 mL and stirred for 1 hour at room temperature. The resulting mixture was quenched with sat. NaHCO₃, extracted 3 times with EtOAc and the organic layer was collected, dried over anhydrous Na₂SO₄, filtered, and evaporated to dryness.

The crude residue was purified by silica gel chromatography (0 to 100% MeOH/DCM). The fractions containing the product were collected and the solvent was removed under reduced pressure to provide the title compound. LCMS Method C, RT=4.542 min, m/z=390.1 [M+H]⁺, LCMS Method D, RT=4.189 min, m/z=390.1276 [M+H]⁺, exact mass: 389.1198.

### Example 60: (S)-5-((((5-(4-(2,3-dihydrobenzo[b][1,4]dioxin-6-yl)-1H-indol-1-yl)-3-methoxypyrazin-2-yl)methyl)amino)methylpyrrolidin-2-one

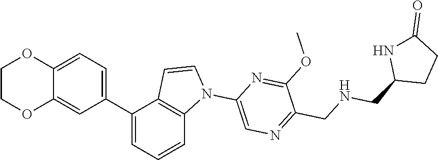 [SMILES: COc1nc(-n2ccc3c(-c4ccc5c(c4)OCCO5)cccc32)cnc1CNC[C@@H]1CCC(=O)N1]

### Step 1: 5-bromo-2-iodo-3-methoxypyrazine

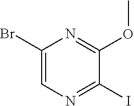

Isoamyl nitrite (10.6 mL, 78.9 mmol) was added to a mixture of 5-bromo-3-methoxypyrazin-2-amine (4.00 g, 19.6 mmol), CuI (1.18 g, 6.20 mmol), 12 (2.53 g, 9.97 mmol) and KI (3.38 g, 20.4 mmol) in DME 75 mL at rt before the reaction mixture was stirred at 60° C. for 1 hour. Then, the mixture was cooled down, evaporated to dryness and purified by column chromatography (silica gel, gradient elution, 0 to 20% EtOAc/hexanes) to provide 5-bromo-2-iodo-3-methoxypyrazine (1.90 g). LCMS Method C, RT=5.138 min, m/z=314.9 [M+H]⁺, exact mass: 313.8552.

### Step 2: 5-bromo-3-methoxypyrazine-2-carbaldehyde

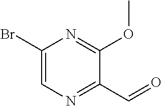

n-BuLi (2.0 mL, 2M in THF) was slowly added to a solution of 5-bromo-2-iodo-3-methoxypyrazine (1.01 g, 3.21 mmol) in THF 30 mL at −78° C. under argon atmosphere. After it was stirred at −78° C. for 10 min, DMF (2.6 mL, 33.6 mmol) was added. The reaction mixture was stirred at −78° C. for 90 min before stirring at 0° C. for 30 min. Then sat·NaHCO₂ was slowly added to the reaction mixture at 0° C. The resulting mixture was extracted 3 times with EtOAc and the organic layer was collected, dried over anhydrous Na₂SO₄, filtered, and evaporated to dryness. The crude mixture was purified by column chromatography (silica gel, gradient elution, 0 to 50% EtOAc/hexanes) to provide 5-bromo-3-methoxypyrazine-2-carbaldehyde (135.2 mg). LCMS Method C, RT=4.270 min, m/z=217.0 [M+H]⁺, exact mass: 215.9534.

### Step 3: 5-(4-(2,3-dihydrobenzo[b][1,4]dioxin-6-yl)-1H-indol-1-yl)-3-methoxypyrazine-2-carbaldehyde

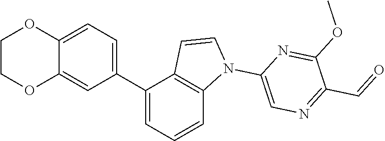 [SMILES: COc1nc(-n2ccc3c(-c4ccc5c(c4)OCCO5)cccc32)cnc1C=O]

A mixture of Intermediate A1 (294.4 mg, 1.17 mmol), 5-bromo-3-methoxypyrazine-2-carbaldehyde (165.1 mg, 0.76 mmol), Pd₂(dba)₃ (21.9 mg, 0.02 mmol), Xantphos (46.8 mg, 0.08 mmol) and Cs₂CO₃ (769.8 mg, 2.36 mmol) in toluene 8 mL was sparged with argon for 10 min before it was refluxed for overnight under argon atmosphere. Then, the mixture was cooled down and diluted with water. The resulting mixture was extracted 3 times with EtOAc and the organic layer was collected, dried over anhydrous Na₂SO₄, filtered, and evaporated to dryness. The crude mixture was purified by column chromatography (silica gel, gradient elution, 20 to 100% EtOAc/hexanes) to 5-(4-(2,3-dihydrobenzo[b][1,4]dioxin-6-yl)-1H-indol-1-yl)-3-methoxypyrazine-2-carbaldehyde (35.9 mg). LCMS Method C, RT=5.335 min, m/z=388.1 [M+H]⁺, exact mass: 387.1219.

### Step 4: (S)-5-((((5-(4-(2,3-dihydrobenzo[b][1,4]dioxin-6-yl)-1H-indol-1-yl)-3-methoxypyrazin-2-yl)methyl)amino)methyl)pyrrolidin-2-one

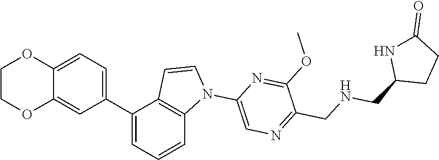 [SMILES: COc1nc(-n2ccc3c(-c4ccc5c(c4)OCCO5)cccc32)cnc1CNC[C@@H]1CCC(=O)N1]

A mixture of 4-(2,3-dihydrobenzo[b][1,4]dioxin-6-yl)-1′-methyl-1′H-[1,6′-biindole]-2′-carbaldehyde (35.9 mg, 0.09 mmol) and (S)-5-(aminomethyl)pyrrolidin-2-one (15.0 mg, 0.13 mmol) in 10 mL MeOH/DCM (1:1) was stirred at room temperature. Then, acetic acid was added until the pH value around 4 before the mixture was left for 1 hour. Sodium cyanoborohydride (30.0 mg, 0.44 mmol) was added and the reaction was stirred at room temperature for overnight. The reaction mixture was purified by column chromatography (silica gel, gradient elution, 0 to 100% MeOH/DCM). The fractions containing the product were collected and the solvent was removed under reduced pressure to provide the title compound. LCMS Method C, RT=4.286 min, m/z=486.2 [M+H]⁺, LCMS Method D, RT=3.989 min, m/z=486.2103 [M+H]⁺, exact mass: 485.2063.

### Example 61: (S)-5-((((5-(4-(2,3-dihydrobenzo[b][1,4]dioxin-6-yl)-1H-indol-1-yl)-3-methoxypyridin-2-yl)methyl)amino)methyl)pyrrolidin-2-one

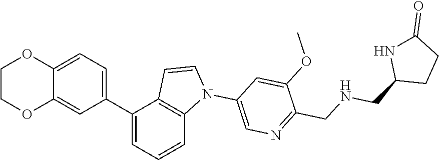 [SMILES: COc1cc(-n2ccc3c(-c4ccc5c(c4)OCCO5)cccc32)cnc1CNC[C@@H]1CCC(=O)N1]

This compound was prepared and purified by using similar procedures as describe in Step 3 of Example 60 replacing 5-bromo-3-methoxypyrazine-2-carbaldehyde to -bromo-3-methoxypicolinaldehydein to provide an intermediate 5-(4-(2,3-dihydrobenzo[b] [1,4]dioxin-6-yl)-1H-indol-1-yl)-3-methoxypicolinaldehyde. LCMS Method C, RT=5.091 min, m/z=387.1 [M+H]⁺, exact mass: 386.1267. Then, the intermediate was processed till purification by following Step 4 of Example 60 resulting in the title compound. LCMS Method C, RT=4.206 min, m/z=485.3 [M+H]⁺, LCMS Method D, RT=3.897 min, m/z=485.2154 [M+H]⁺, exact mass: 484.2111.

### Example 62: (S)-5-(((2-(5-(4-(2,3-dihydrobenzo[b] [1,4]dioxin-6-yl)-1H-indol-1-yl)-3-methyl-1H-indazol-1-yl)ethyl)amino)methyl)pyrrolidin-2-one

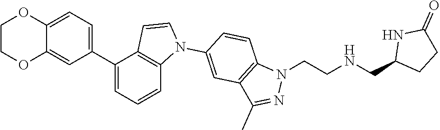 [SMILES: Cc1nn(CCNC[C@@H]2CCC(=O)N2)c2ccc(-n3ccc4c(-c5ccc6c(c5)OCCO6)cccc43)cc12]

### Step 1: 5-(4-(2,3-dihydrobenzo[b][1,4]dioxin-6-yl)-1H-indol-1-yl)-3-methyl-1H-indazole

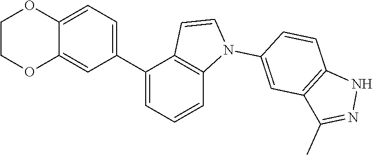 [SMILES: Cc1n[nH]c2ccc(-n3ccc4c(-c5ccc6c(c5)OCCO6)cccc43)cc12]

A mixture of Intermediate A1 (0.40 g, 1.86 mmol), tert-butyl 5-bromo-3-methyl-1H-indazole-1-carboxylate (0.69 g, 2.22 mmol), Pd₂(dba)₃ (0.09 g, 0.09 mmol), Xantphos (0.11 g, 0.19 mmol) and Cs₂CO₃ (2.12 g, 6.51 mmol) in toluene 15 mL was sparged with argon for 10 min before it was refluxed under argon atmosphere for overnight. Then, the mixture was cooled down and diluted with water. The resulting mixture was extracted 3 times with EtOAc and the organic layer was collected, dried over anhydrous Na₂SO₄, filtered, and evaporated to dryness. The crude mixture was purified by column chromatography (silica gel, gradient elution, 0 to 60% EtOAc/hexanes) to provide tert-butyl 5-(4-(2,3-dihydrobenzo[b][1,4]dioxin-6-yl)-1H-indol-1-yl)-3-methyl-1H-indazole-1-carboxylate.

Then, 4M HCl in dioxane (1 mL) was added to this compound and stirred at room temperature for 30 minutes. Then the reaction mixture was evaporated to dryness. The crude mixture was purified by column chromatography (silica gel, gradient elution, 0 to 30% MeOH/DCM) to provide 5-(4-(2,3-dihydrobenzo[b][1,4]dioxin-6-yl)-1H-indol-1-yl)-3-methyl-1H-indazole (0.07 g)

### Step 2: (S)-5-(((2-(5-(4-(2,3-dihydrobenzo[b][1,4]dioxin-6-yl)-1H-indol-1-yl)-3-methyl-1H-indazol-1-yl)ethyl)amino)methyl)pyrrolidin-2-one

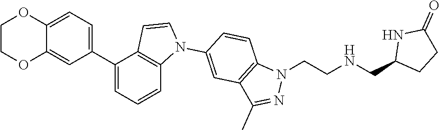 [SMILES: Cc1nn(CCNC[C@@H]2CCC(=O)N2)c2ccc(-n3ccc4c(-c5ccc6c(c5)OCCO6)cccc43)cc12]

1,2-Dibromoethane (0.02 mL, 0.23 mmol) was added to a mixture of 5-(4-(2,3-dihydrobenzo[b][1,4]dioxin-6-yl)-1H-indol-1-yl)-3-methyl-1H-indazole (73.0 mg, 0.19 mmol) and K₂CO₃ (331.6 mg, 2.40 mmol) in DMF 2.5 mL before stirring at 60° C. for overnight. Then a solution of (S)-5-(aminomethyl)pyrrolidin-2-one (36.4 mg, 0.32 mmol) in DMF 1.2 mL was transferred to the reaction mixture at rt. The mixture was stirred at 60° C. for 9 hours. Then, the reaction solution was cooled down and diluted with water. The resulting mixture was extracted 3 times with EtOAc and the organic layer was collected, dried over anhydrous Na₂SO₄, filtered, and evaporated to dryness. The crude mixture was purified by column chromatography (silica gel, gradient elution, 0 to 200% MeOH/DCM) to provide the title compound. LCMS Method C, RT=4.393 min, m/z=522.3 [M+H]⁺, LCMS Method D, RT=4.223 min, m/z=522.2474 [M+H]⁺, exact mass: 521.2427.

### Example 63: (S)-5-((((8-(4-(2,3-dihydrobenzo[b][1,4]dioxin-6-yl)-1H-indol-1-yl)-6-methyl-1,7-naphthyridin-3-yl)methyl)amino)methyl)pyrrolidin-2-one

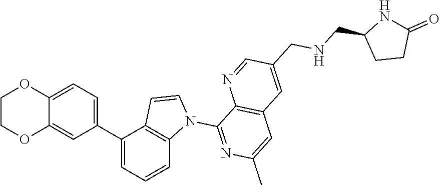 [SMILES: Cc1cc2cc(CNC[C@@H]3CCC(=O)N3)cnc2c(-n2ccc3c(-c4ccc5c(c4)OCCO5)cccc32)n1]

### Step 1: 5-bromo-N-(tert-butyl)-3-methylpicolinamide

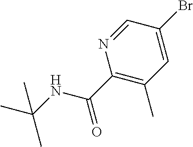

Conc. H₂SO₄ 1 mL was slowly added to a mixture of 5-bromo-3-methylpyridine-2-carbonitrile (2.01 g, 10.2 mmol) and AcOH 1.6 mL in t-BuOH 2 mL at 0° C. The reaction mixture was stirred at room temperature for overnight. Then, the mixture was poured into water and extracted 3 times with EtOAc. The organic layer was collected, dried over anhydrous Na₂SO₄, filtered, and evaporated to dryness. The crude residue was purified by column chromatography (silica gel, gradient elution, 0 to 20% EtOAc/hexanes) to provide 5-bromo-N-(tert-butyl)-3-methylpicolinamide (2.70 g). LCMS Method C, RT=4.856 min, m/z=271.0 [M+H]⁺, exact mass: 270.0368.

### Step 2: 5-bromo-N-(tert-butyl)-3-(2-oxopropyl)picolinamide

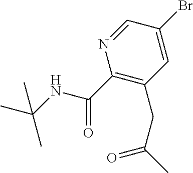 [SMILES: CC(=O)Cc1cc(Br)cnc1C(=O)NC(C)(C)C]

A solution of 5-bromo-N-(tert-butyl)-3-methylpicolinamide (2.59 g, 9.55 mmol) in THF 3 mL was slowly added to LDA solution (23.0 mL of 1M LDA in THF/hexanes dissolved in 10 mL THF) at −40° C. under argon atmosphere. After it was stirred at −40° C. for 15 min, the reaction temperature was warmed up to −10° C. The reaction mixture was transferred to a solution of EtOAc (1.2 mL, 12.3 mmol) in THF 6 mL at −40° C. before stirring at −10° C. for 15 min. Then, the mixture was quenched with sat·NH₄Cl at 0° C. The resulting mixture was extracted 3 times with DCM and the organic layer was collected, dried over anhydrous Na₂SO₄, filtered, and evaporated to dryness. The crude mixture was purified by column chromatography (silica gel, gradient elution, 0 to 30% EtOAc/hexanes) to provide 5-bromo-N-(tert-butyl)-3-(2-oxopropyl)picolinamide (1.39 g). LCMS Method A, RT=3.217 min, m/z=313.0 [M+H]⁺, exact mass: 312.0473.

### Step 3: 3-bromo-6-methyl-1,7-naphthyridin-8-ol

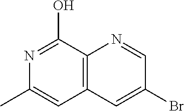

A mixture of 5-bromo-N-(tert-butyl)-3-(2-oxopropyl)picolinamide (1.39 g, 4.44 mmol), AcONH₄ (3.47 g, 45.0 mmol) in AcOH 5 mL was stirred at 108° C. for overnight before it was cooled down and diluted with water. The resulting solid was collected by filtration to provide 3-bromo-6-methyl-1,7-naphthyridin-8-ol (1.44 g). LCMS Method B, RT=2.592 min, m/z=239.0 [M+H]⁺, exact mass: 237.9742.

### Step 4: 3-bromo-8-chloro-6-methyl-1,7-naphthyridine

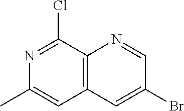

(COCl)₂ (0.62 mL, 7.33 mmol) was added to a solution of 3-bromo-6-methyl-1,7-naphthyridin-8-ol (0.39 g, 1.63 mmol) and DMF (2-3 drops) in 1,2-DCE 10 mL at 0° C. After it was stirred at 80° C. for 3 hours, sat·NaHCO₃ was slowly added to the reaction mixture at rt. The resulting mixture was extracted 3 times with DCM and the organic layer was collected, dried over anhydrous Na₂SO₄, filtered, and evaporated to dryness. The crude mixture was precipitated by using DCM/hexanes to provide 3-bromo-8-chloro-6-methyl-1,7-naphthyridine (0.34 g). LCMS Method B, RT=3.729 min, m/z=256.9 [M+H]⁺, exact mass: 255.9403.

### Step 5: 3-bromo-8-(4-(2,3-dihydrobenzo[b][1,4]dioxin-6-yl)-1H-indol-1-yl)-6-methyl-1,7-naphthyridine

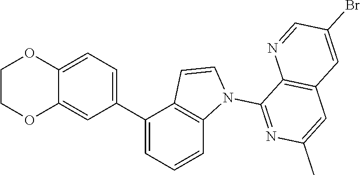 [SMILES: Cc1cc2cc(Br)cnc2c(-n2ccc3c(-c4ccc5c(c4)OCCO5)cccc32)n1]

A mixture of Intermediate A1 (0.20 g, 0.80 mmol), 3-bromo-8-chloro-6-methyl-1,7-naphthyridine (0.20 g, 0.78 mmol) and Cs₂CO₃ (0.79 g, 2.42 mmol) in DMF 8 mL was stirred at 80° C. for overnight. Then, the mixture was cooled down and diluted with water. The resulting mixture was extracted 3 times with EtOAc and the organic layer was collected, dried over anhydrous Na₂SO₄, filtered, and evaporated to dryness. The crude mixture was purified by column chromatography (silica gel, gradient elution, 0 to 50% EtOAc/hexanes) to provide 3-bromo-8-(4-(2,3-dihydrobenzo[b] [1,4]dioxin-6-yl)-1H-indol-1-yl)-6-methyl-1,7-naphthyridine (0.13 g). LCMS Method B, RT=5.323 min, m/z=472.1 [M+H]⁺, exact mass: 471.0582.

### Step 6: 8-(4-(2,3-dihydrobenzo[b][1,4]dioxin-6-yl)-1H-indol-1-yl)-6-methyl-1,7-naphthyridine-3-carbaldehyde

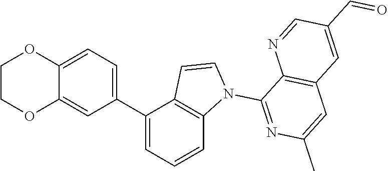 [SMILES: Cc1cc2cc(C=O)cnc2c(-n2ccc3c(-c4ccc5c(c4)OCCO5)cccc32)n1]

n-BuLi (0.34 mL, 2M in THF) was slowly added to a solution of 3-bromo-8-(4-(2,3-dihydrobenzo[b] [1,4]dioxin-6-yl)-1H-indol-1-yl)-6-methyl-1,7-naphthyridine (0.13 g, 0.28 mmol) in THF 3 mL at −78° C. under argon atmosphere. After it was stirred at −78° C. for 15 min, DMF (0.24 mL, 3.10 mmol) was added. The reaction mixture was stirred at −78° C. for 1 hour and quenched with sat·NH₄Cl at 0° C. The resulting mixture was extracted 3 times with EtOAc and the organic layer was collected, dried over anhydrous Na₂SO₄, filtered, and evaporated to dryness. The crude mixture was purified by column chromatography (silica gel, gradient elution, 0 to 30% EtOAc/hexanes) to provide 8-(4-(2,3-dihydrobenzo[b] [1,4]dioxin-6-yl)-1H-indol-1-yl)-6-methyl-1,7-naphthyridine-3-carbaldehyde (0.03 g). LCMS Method B, RT=4.654 min, m/z=422.2 [M+H]⁺, exact mass: 421.1426.

### Step 7. (S)-5-((((8-(4-(2,3-dihydrobenzo[b][1,4]dioxin-6-yl)-1H-indol-1-yl)-6-methyl-1,7-naphthyridin-3-yl)methyl)amino)methyl)pyrrolidin-2-one

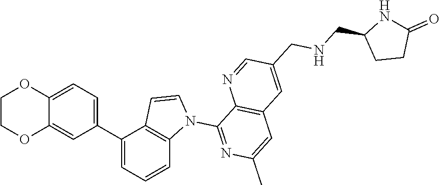 [SMILES: Cc1cc2cc(CNC[C@@H]3CCC(=O)N3)cnc2c(-n2ccc3c(-c4ccc5c(c4)OCCO5)cccc32)n1]

A mixture of 8-(4-(2,3-dihydrobenzo[b][1,4]dioxin-6-yl)-1H-indol-1-yl)-6-methyl-1,7-naphthyridine-3-carbaldehyde (32.0 mg, 0.08 mmol) and (S)-5-(aminomethyl)pyrrolidin-2-one (11.1 mg, 0.10 mmol) in 3 mL MeOH/DCM (1:1) was stirred at room temperature and acetic acid was added until the pH was around 4. After the reaction was stirred for 1 hour, sodium cyanoborohydride (25.9 mg, 0.38 mmol) was added and the reaction was stirred at room temperature for overnight. The reaction mixture was purified by column chromatography (silica gel, gradient elution, 0 to 30% MeOH/DCM). The fractions containing the product were collected and the solvent was removed under reduced pressure to provide the title compound. LCMS Method B, RT=3.553 min, m/z=520.2 [M+H]⁺, LCMS Method D, RT=4.105 min, m/z=520.2326 [M+H]⁺, exact mass: 519.2270.

### Example 64: (5S,5′S)-5,5′-((((1H,1′H-[4,4′-biindole]-1,1′-diylbis(2,6-dimethoxy-4,1-phenylene))bis(methylene))bis(azanediyl))bis(methylene))bis(pyrrolidin-2-one)

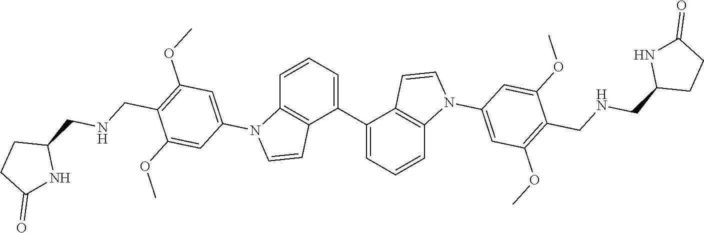 [SMILES: COc1cc(-n2ccc3c(-c4cccc5c4ccn5-c4cc(OC)c(CNC[C@@H]5CCC(=O)N5)c(OC)c4)cccc32)cc(OC)c1CNC[C@@H]1CCC(=O)N1]

### Step 1: 4-(4,4,5,5-tetramethyl-1,3,2-dioxaborolan-2-yl)-1H-indole

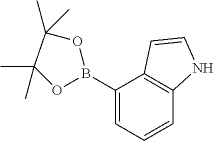 [SMILES: CC1(C)OB(c2cccc3[nH]ccc23)OC1(C)C]

A mixture of 4-bromoindole (4.01 g, 20.5 mmol), B₂Pin₂ (6.22 g, 24.5 mmol), Pd₂(dba)₃ (0.38 g, 0.41 mmol), X-Phos (0.39 g, 0.82 mmol) and KOAc (6.01 g, 61.2 mmol) in 1,4-dioxane 40 mL was sparged with argon for 10 min before stirring at 110° C. for overnight under argon atmosphere. Then, the mixture was cooled down and diluted with brine. The resulting mixture was extracted 3 times with EtOAc and the organic layer was collected, dried over anhydrous Na₂SO₄, filtered, and evaporated to dryness. The crude residue was precipitated by DCM/hexanes to provide 4-(4,4,5,5-tetramethyl-1,3,2-dioxaborolan-2-yl)-1H-indole (3.58 g). LCMS Method C, RT=4.771 min, m/z=244.1 [M+H]⁺, exact mass: 243.1431.

### Step 2: 1H,1′H-4,4′-biindole

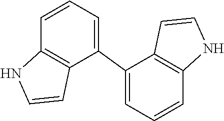 [SMILES: c1cc(-c2cccc3[nH]ccc23)c2cc[nH]c2c1]

A mixture of 4-(4,4,5,5-tetramethyl-1,3,2-dioxaborolan-2-yl)-1H-indole (0.80 g, 3.29 mmol), 4-bromoindole (0.65 g, 3.31 mmol), Pd(PPh₃)₄ (0.38 g, 0.33 mmol) and K₂CO₇ (1.15 g, 8.32 mmol) in a mixture of 1,4-dioxane and H₂O (6:1) 14 mL was sparged with argon for 10 min before stirring at 100° C. for 24 hours under argon atmosphere. Then, the mixture was cooled down, filtered and evaporated to dryness. The crude residue was precipitated by DCM/acetone/hexanes to provide 1H,1′H-4,4′-biindole (0.18 g). LCMS Method C, RT=4.812 min, m/z=233.1 [M+H]⁺, exact mass: 232.1000.

### Step 3: 4,4′-(1H,1′H-[4,4′-biindole]-1,1′-diyl)bis(2,6-dimethoxybenzaldehyde)

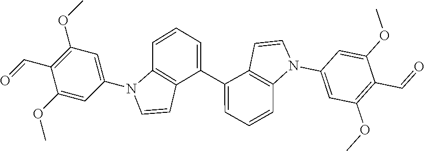 [SMILES: COc1cc(-n2ccc3c(-c4cccc5c4ccn5-c4cc(OC)c(C=O)c(OC)c4)cccc32)cc(OC)c1C=O]

A mixture of 1H,1′H-4,4′-biindole (0.18 g, 0.77 mmol), 4-bromo-2,6-dimethoxybenzaldehyde (0.57 g, 2.33 mmol), Pd(dba)₃ (0.04 g, 0.04 mmol), Xantphos (0.09 g, 0.16 mmol) and Cs₂CO₃ (1.05 g, 3.22 mmol) in toluene 7 mL was sparged with argon for 10 min before getting reflux under argon atmosphere for overnight. The resulting mixture was cooled down, filtered, and evaporated to dryness. The crude mixture was purified by column chromatography (silica gel, gradient elution, 20 to 80% EtOAc/hexanes) to provide 4,4′-(1H,1′H-[4,4′-biindole]-1,1′-diyl)bis(2,6-dimethoxybenzaldehyde) (0.09 g). LCMS Method C, RT=5.906 min, m/z=561.3 [M+H]⁺, exact mass: 560.1947.

### Step 4: (5S,5'S)-5,5′-((((1H,1′H-[4,4′-biindole]-1,1′-diylbis(2,6-dimethoxy-4,1-phenylene))bis(methylene))bis(azanediyl))bis(methylene))bis(pyrrolidin-2-one)

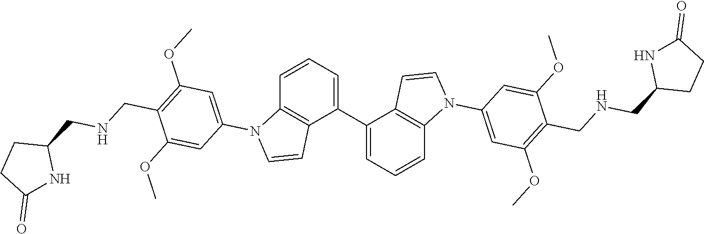 [SMILES: COc1cc(-n2ccc3c(-c4cccc5c4ccn5-c4cc(OC)c(CNC[C@@H]5CCC(=O)N5)c(OC)c4)cccc32)cc(OC)c1CNC[C@@H]1CCC(=O)N1]

A mixture of 4,4′-(1H,1′H-[4,4′-biindole]-1,1′-diyl)bis(2,6-dimethoxybenzaldehyde) (93.7 mg, 0.17 mmol) and (S)-5-(aminomethyl)pyrrolidin-2-one (61.5 mg, 0.54 mmol) in 8 mL MeOH/DCM (1:1) was stirred at room temperature and acetic acid was added until the pH was around 4. After the reaction was stirred for 1 hour, sodium cyanoborohydride (101.0 mg, 1.47 mmol) was added and the reaction was stirred at room temperature for overnight. The solvent was evaporated and the mixture was purified by column chromatography (silica gel, gradient elution, 0 to 20% MeOH/DCM). The fractions containing the product were collected and the solvent was removed under reduced pressure, resulting in the title compound. LCMS Method C, RT=3.970 min, m/z=757.5 [M+H]⁺, LCMS Method D, RT=3.739 min, m/z=757.3709 [M+H]⁺, exact mass: 756.3635.

### Example 65: (S)-5-((((5-(2-chloro-3-(1-(3,5-dimethoxy-4-(((((S)-5-oxopyrrolidin-2-yl)methyl)amino)methyl)phenyl)-1H-indol-4-yl)phenyl)-3-methoxypyrazin-2-yl)methyl)amino)methyl)pyrrolidin-2-one

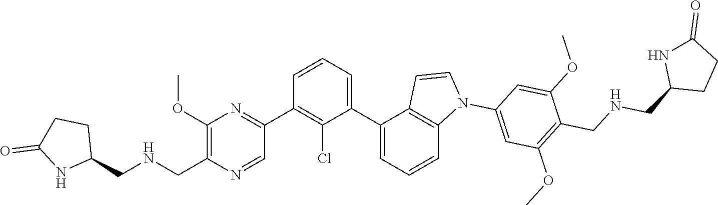 [SMILES: COc1cc(-n2ccc3c(-c4cccc(-c5cnc(CNC[C@@H]6CCC(=O)N6)c(OC)n5)c4Cl)cccc32)cc(OC)c1CNC[C@@H]1CCC(=O)N1]

### Step 1: 5-(2-chloro-3-(I-(4-formyl-3,5-dimethoxyphenyl)-1H-indol-4-yl)phenyl)-3-methoxypyrazine-2-carbaldehyde

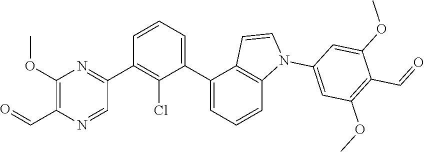 [SMILES: COc1cc(-n2ccc3c(-c4cccc(-c5cnc(C=O)c(OC)n5)c4Cl)cccc32)cc(OC)c1C=O]

A mixture of 2-chloro-1,3-dibromobenzene (0.66 g, 2.44 mmol), 4-(4,4,5,5-tetramethyl-1,3,2-dioxaborolan-2-yl)indoline (0.49 g, 2.02 mmol), Pd(dppf)Cl₂·CH₂Cl₂ (0.08 g, 0.10 mmol) and K₂CO₃ (0.84 g, 6.08 mmol) in a mixture of 1,4-dioxane and H₂O (3:1) 8 mL was sparged with argon for 10 min before stirring at 100° C. for overnight under argon atmosphere. Then, the mixture was cooled down, filtered, and evaporated to dryness. The crude residue was purified by column chromatography (silica gel, gradient elution, 0 to 30% EtOAc/hexanes) to provide 4-(3-bromo-2-chlorophenyl)-1H-indole (0.42 g). LCMS Method C, RT=5.404 min, m/z=306.0 [M+H]⁺, exact mass: 304.9607.

Next, a mixture of 4-(3-bromo-2-chlorophenyl)-1H-indole (0.34 g, 1.11 mmol), B₂Pin₂ (0.34 g, 1.34 mmol), Pd(dppf)Cl₂·CH₂CO₂ (0.05 g, 0.06 mmol) and KOAc (0.76 g, 7.74 mmol) in DMF 10 mL was sparged with argon for 10 min before stirring at 110° C. for 2 hours under argon atmosphere. Then, the mixture was cooled down and diluted with sat. NaHCO₃. The resulting mixture was extracted 3 times with EtOAc and the organic layer was collected, dried over anhydrous Na₂SO₄, filtered, and evaporated to dryness. The crude residue was purified by column chromatography (silica gel, gradient elution, 0 to 30% EtOAc/hexanes) to provide 4-(2-chloro-3-(4,4,5,5-tetramethyl-1,3,2-dioxaborolan-2-yl)phenyl)-1H-indole (0.22 g). LCMS Method C, RT=5.574 min, m/z=354.1 [M+H]⁺, exact mass: 353.1354.

A mixture of 4-(2-chloro-3-(4,4,5,5-tetramethyl-1,3,2-dioxaborolan-2-yl)phenyl)-1H-indole (0.22 g, 0.62 mmol), 5-bromo-3-methoxypyrazine-2-carbaldehyde (0.15 g, 0.69 mmol), Pd(dppf)Cl₂·CH₂Cl₂ (0.03 g, 0.04 mmol) and K₂CO₃ (0.31 g, 2.24 mmol) in a mixture of 1,4-dioxane and H₂O (3:1) 8 mL was sparged with argon for 10 min before stirring at 100° C. for overnight under argon atmosphere. Then, the mixture was cooled down and diluted with sat·NaHCO₃. The resulting mixture was extracted 3 times with EtOAc and the organic layer was collected, dried over anhydrous Na₂SO₄, filtered, and evaporated to dryness.

A mixture of this crude mixture, 4-bromo-2,6-dimethoxybenzaldehyde (0.19 g, 0.78 mmol), Pd₂(dba)₃ (0.02 g, 0.02 mmol), Xantphos (0.04 g, 0.07 mmol) and Cs₂CO₃ (0.45 g, 1.38 mmol) in toluene 10 mL was sparged with argon for 10 min before it was refluxed for 2 days under argon atmosphere. Then, the mixture was cooled down, filtered, and evaporated to dryness. The crude residue was purified by column chromatography (silica gel, gradient elution, 0 to 50% EtOAc/hexanes) to provide 5-(2-chloro-3-(1-(4-formyl-3,5-dimethoxyphenyl)-1H-indol-4-yl)phenyl)-3-methoxypyrazine-2-carbaldehyde (0.04 g). LCMS Method C, RT=5.615 min, m/z=528.2 [M+H]⁺, exact mass: 527.1248.

### Step 2: (S)-5-((((5-(2-chloro-3-(1-(3,5-dimethoxy-4-(((((S)-5-oxopyrrolidin-2-yl)methyl)amino)methyl)phenyl)-1H-indol-4-yl)phenyl)-3-methoxypyrazin-2-yl)methyl)amino) methyl)pyrrolidin-2-one

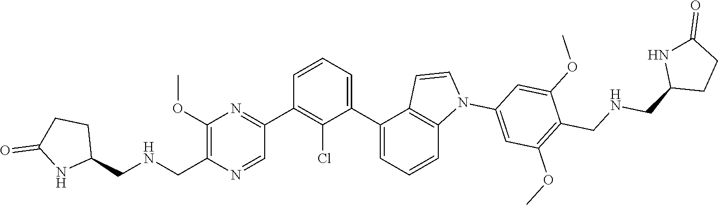 [SMILES: COc1cc(-n2ccc3c(-c4cccc(-c5cnc(CNC[C@@H]6CCC(=O)N6)c(OC)n5)c4Cl)cccc32)cc(OC)c1CNC[C@@H]1CCC(=O)N1]

A mixture of 5-(2-chloro-3-(1-(4-formyl-3,5-dimethoxyphenyl)-1H-indol-4-yl)phenyl)-3-methoxypyrazine-2-carbaldehyde (37.2 mg, 0.07 mmol) and (S)-5-(aminomethyl)pyrrolidin-2-one (24.6 mg, 0.22 mmol) in 4 mL MeOH/DCM (1:1) was stirred at room temperature and acetic acid was added until the pH was around 4. After the reaction was stirred for 1 hour, sodium cyanoborohydride (40.2 mg, 0.58 mmol) was added and the reaction was stirred at room temperature for overnight. The reaction mixture was purified by column chromatography (silica gel, gradient elution, 0 to 30% MeOH/DCM). The fractions containing the product were collected and the solvent was removed under reduced pressure to provide the title compound. LCMS Method C, RT=3.971 min, m/z=724.4 [M+H]⁺, LCMS Method D, RT=3.535 min, m/z=724.3002 [M+H]⁺, exact mass: 723.2936.

### Example 66: (S)-1-(((4-(4-(2-chloro-3-(6-methoxy-5-((((5-oxopyrrolidin-2-yl)methyl)amino)methyl)pyridin-2-yl)phenyl)-1H-indazol-1-yl)-2,6-dimethoxybenzyl)amino)methyl)cyclopropanecarboxylic acid

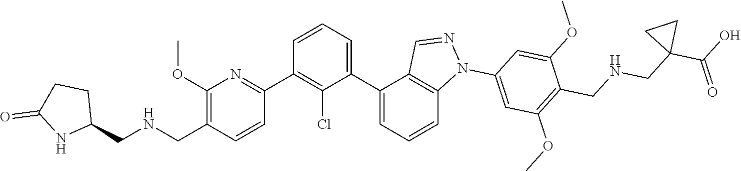 [SMILES: COc1cc(-n2ncc3c(-c4cccc(-c5ccc(CNC[C@@H]6CCC(=O)N6)c(OC)n5)c4Cl)cccc32)cc(OC)c1CNCC1(C(=O)O)CC1]

### Step 1: 1-(((4-(4-bromo-1H-indazol-1-yl)-2,6-dimethoxybenzyl)amino)methyl)cyclopropanecarboxylic acid

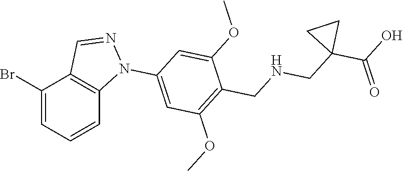 [SMILES: COc1cc(-n2ncc3c(Br)cccc32)cc(OC)c1CNCC1(C(=O)O)CC1]

A mixture of Intermediate B4 (0.10 g, 0.28 mmol) and 1-(aminomethyl)cyclopropane carboxylic acid hydrochloride (0.06 g, 0.40 mmol) in 8 mL MeOH/DCM (1:1) was stirred at room temperature and acetic acid was added until the pH was around 4. After the reaction was stirred for 1 hour, sodium cyanoborohydride (0.08 g, 1.16 mmol) was added and the reaction was stirred at room temperature for overnight. The reaction mixture was purified by column chromatography (silica gel, gradient elution, 0 to 40% MeOH/DCM). The fractions containing the product were collected and the solvent was removed under reduced pressure to provide 1-(((4-(4-bromo-1H-indazol-1-yl)-2,6-dimethoxybenzyl)amino)methyl)cyclopropanecarboxylic acid (0.07 g).

### Step 2: 1-(((4-(4-(2-chloro-3-(5-formyl-6-methoxypyridin-2-yl)phenyl)-1H-indazol-1-yl)-2,6-dimethoxybenzyl)amino)methyl)cyclopropanecarboxylic acid

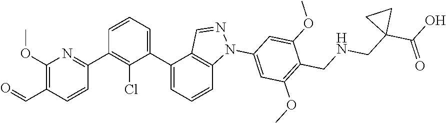 [SMILES: COc1cc(-n2ncc3c(-c4cccc(-c5ccc(C=O)c(OC)n5)c4Cl)cccc32)cc(OC)c1CNCC1(C(=O)O)CC1]

A mixture of Intermediate C2 (0.07 g, 0.19 mmol), 1-(((4-(4-bromo-1H-indazol-1-yl)-2,6-dimethoxybenzyl)amino)methyl)cyclopropanecarboxylic acid (0.07 g, 0.15 mmol), Pd(PPh₃)₄ (0.02 g, 0.02 mmol) and K₂CO₃ (0.07 g, 0.51 mmol) in a mixture of 1,4-dioxane and H₂O (6:1) 10 mL was sparged with argon for 10 min before stirring at 95° C. for 2 hours under argon atmosphere. Then, the mixture was cooled down and diluted with brine. The resulting mixture was extracted 3 times with EtOAc and the organic layer was collected, dried over anhydrous Na₂SO₄, filtered, and evaporated to dryness. The reaction mixture was purified by column chromatography (silica gel, gradient elution, 0 to 100% MeOH/DCM) to provide 1-(((4-(4-(2-chloro-3-(5-formyl-6-methoxypyridin-2-yl)phenyl)-1H-indazol-1-yl)-2,6-dimethoxybenzyl)amino)methyl)cyclopropanecarboxylic acid (0.05 g). LCMS Method B, RT=4.015 min, m/z=627.3 [M+H]⁺, exact mass: 626.1932.

### Step 3: (S)-1-(((4-(4-(2-chloro-3-(6-methoxy-5-((((5-oxopyrrolidin-2-yl)methyl)amino)methyl)pyridin-2-yl)phenyl)-1H-indazol-1-yl)-2,6-dimethoxybenzyl)amino) methyl)cyclopropanecarboxylic acid

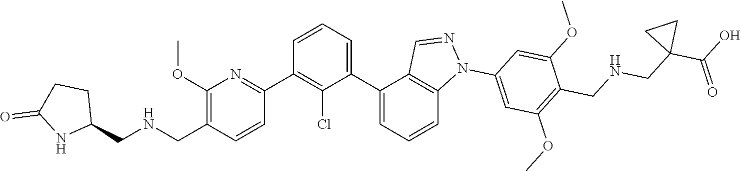 [SMILES: COc1cc(-n2ncc3c(-c4cccc(-c5ccc(CNC[C@@H]6CCC(=O)N6)c(OC)n5)c4Cl)cccc32)cc(OC)c1CNCC1(C(=O)O)CC1]

A mixture of 1-(((4-(4-(2-chloro-3-(5-formyl-6-methoxypyridin-2-yl)phenyl)-1H-indazol-1-yl)-2,6-dimethoxybenzyl)amino)methyl)cyclopropanecarboxylic acid (50.0 mg, 0.08 mmol) and (S)-5-(aminomethyl)pyrrolidin-2-one (13.5 mg, 0.12 mmol) in 4 mL MeOH/DCM (1:1) was stirred at room temperature and acetic acid was added until the pH was around 4. After the reaction was stirred for 1 hour, sodium cyanoborohydride (25.5 mg, 0.37 mmol) was added and the reaction was stirred at room temperature for 2 hours. The crude mixture was purified by column chromatography (silica gel, gradient elution, 0 to 40% MeOH/DCM). The fractions containing the product were collected and the solvent was removed under reduced pressure to provide the title compound. LCMS Method B, RT=3.107 min, m/z=725.3 [M+H]⁺, LCMS Method D, RT=3.606 min, m/z=725.2830 [M+H]⁺, exact mass: 724.2776.

### Example 67: (S)—N-(3-(1-(3,5-dimethoxy-4-((((5-oxopyrrolidin-2-yl)methyl)amino)methyl)phenyl)-1H-indol-4-yl)-2-methylphenyl)-5-methyl-4,5,6,7-tetrahydrothiazolo[5,4-c]pyridine-2-carboxamide

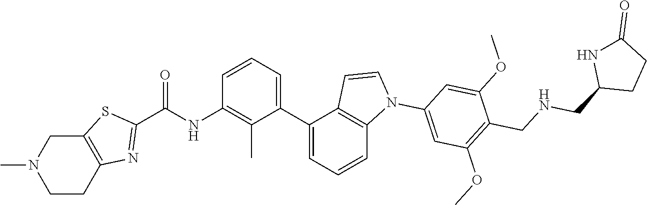 [SMILES: COc1cc(-n2ccc3c(-c4cccc(NC(=O)c5nc6c(s5)CN(C)CC6)c4C)cccc32)cc(OC)c1CNC[C@@H]1CCC(=O)N1]

### Step 1: N-(3-bromo-2-methylphenyl)-5-methyl-4,5,6,7-tetrahydrothiazolo[5,4-c]pyridine-2-carboxamide

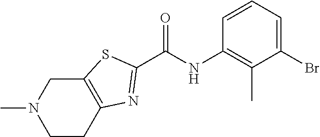 [SMILES: Cc1c(Br)cccc1NC(=O)c1nc2c(s1)CN(C)CC2]

A mixture of 4,5,6,7-tetrahydro-5-methyl-thiazolo[5,4-c]pyridine-2-carboxylic acid hydrochloride (0.82 g, 3.49 mmol) in DCM 25 mL was slowly added to a mixture of 3-bromo-2-methylaniline (0.50 g, 2.68 mmol), HATU (1.84 g, 4.84 mmol) and DIPEA (2.0 mL, 11.5 mmol) in DCM 10 mL before stirring at 40° C. for 4 hours. Then, the mixture was stirred at rt overnight. The resulting mixture was evaporated to dryness and purified by column chromatography (silica gel, gradient elution, 0 to 30% MeOH/DCM) to provide N-(3-bromo-2-methylphenyl)-5-methyl-4,5,6,7-tetrahydrothiazolo[5,4-c]pyridine-2-carboxamide (0.92 g). LCMS Method B, RT=3.500 min, m/z=366.0 [M+H]⁺, exact mass: 365.0197.

### Step 2: N-(3-(1-(4-formyl-3,5-dimethoxyphenyl)-1H-indol-4-yl)-2-methylphenyl)-5-methyl-4,5,6,7-tetrahydrothiazolo[5,4-c]pyridine-2-carboxamide

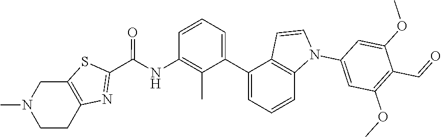 [SMILES: COc1cc(-n2ccc3c(-c4cccc(NC(=O)c5nc6c(s5)CN(C)CC6)c4C)cccc32)cc(OC)c1C=O]

A mixture of N-(3-bromo-2-methylphenyl)-5-methyl-4,5,6,7-tetrahydrothiazolo[5,4-c]pyridine-2-carboxamide (0.24 g, 0.66 mmol), 4-(4,4,5,5-tetramethyl-1,3,2-dioxaborolan-2-yl)indoline (0.22 g, 0.90 mmol), Pd(dppf)Cl₂—CH₂Cl₂ (0.06 g, 0.07 mmol) and K₂CO₃ (0.19 g, 1.37 mmol) in a mixture of 1,4-dioxane and H₂O (10:1) 6.6 mL was sparged with argon for 10 min before it was stirred at 90° C. for 4 hours under argon atmosphere. Then, the mixture was cooled down, filtered, and evaporated to dryness to obtain crude mixture.

4-bromo-2,6-dimethoxybenzaldehyde (0.21 g, 0.86 mmol), Pd₂(dba)₃ (0.03 g, 0.03 mmol), Xantphos (0.04 g, 0.07 mmol) and Cs₂CO₃ (0.45 g, 1.38 mmol) in toluene 12 mL was added to the crude mixture and sparged with argon for 10 min before getting reflux for overnight under argon atmosphere. Then, the solution was cooled down, filtered, and evaporated to dryness. The crude residue was purified by column chromatography (silica gel, gradient elution, 0 to 50% MeOH/DCM) to provide N-(3-(I-(4-formyl-3,5-dimethoxyphenyl)-1H-indol-4-yl)-2-methylphenyl)-5-methyl-4,5,6,7-tetrahydrothiazolo[5,4-c]pyridine-2-carboxamide (0.14 g). LCMS Method C, RT=4.423 min, m/z=567.3 [M+H]⁺, exact mass: 566.1988.

### Step 3: (S)—N-(3-(1-(3,5-dimethoxy-4-((((5-oxopyrrolidin-2-yl)methyl)amino)methyl)phenyl)-1H-indol-4-yl)-2-methylphenyl)-5-methyl-4,5,6,7-tetrahydrothiazolo[5,4-c]pyridine-2-carboxamide

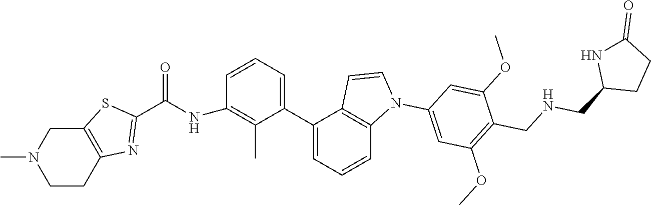 [SMILES: COc1cc(-n2ccc3c(-c4cccc(NC(=O)c5nc6c(s5)CN(C)CC6)c4C)cccc32)cc(OC)c1CNC[C@@H]1CCC(=O)N1]

A mixture of N-(3-(1-(4-formyl-3,5-dimethoxyphenyl)-1H-indol-4-yl)-2-methylphenyl)-5-methyl-4,5,6,7-tetrahydrothiazolo[5,4-c]pyridine-2-carboxamide (138.0 mg, 0.24 mmol) and (S)-5-(aminomethyl)pyrrolidin-2-one (34.2 mg, 0.30 mmol) in 6 mL MeOH/DCM (1:1) was stirred at room temperature and acetic acid was added until the pH was around 4. After the reaction was stirred for 1 hour, sodium cyanoborohydride (69.0 mg, 1.00 mmol) was added and the reaction was stirred at room temperature for 2 hours. The crude mixture was purified by column chromatography (silica gel, gradient elution, 0 to 30% MeOH/DCM). The fractions containing the product were collected and the solvent was removed under reduced pressure to provide the title compound. LCMS Method C, RT=3.890 min, m/z=665.4 [M−H]⁺, LCMS Method D, RT=3.479 min, m/z=665.2904 [M+H]⁺, exact mass: 664.2832.

### Example 68: 1-(4-(4-(2-chloro-3-(5-((((cis-3-hydroxycyclobutyl)methyl)amino)methyl)-6-methoxypyridin-2-yl)phenyl)-1H-indazol-1-yl)-2,6-dimethoxybenzyl)-3-methylazetidin-3-ol

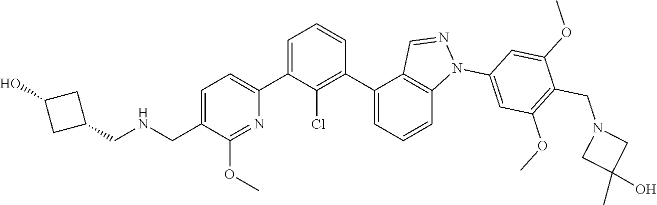 [SMILES: COc1cc(-n2ncc3c(-c4cccc(-c5ccc(CNC[C@H]6C[C@@H](O)C6)c(OC)n5)c4Cl)cccc32)cc(OC)c1CN1CC(C)(O)C1]

A mixture of Intermediate E4 (50 mg, 0.07 mmol) and 3-methylazetidin-3-ol hydrochloride (9.88 mg, 0.08 mmol) in 6 mL MeOH/DCM (1:1) was stirred at room temperature and acetic acid was added until the pH was around 4. After the reaction was stirred for 1 hour, sodium cyanoborohydride (21.99 mg, 0.35 mmol) was added and the reaction was stirred at room temperature for an additional 1 hour. The crude mixture was further used without purification.

The residue was added with 4M HCl in dioxane (1 mL) and stirred at room temperature for 30 minutes. The residual solvent was removed under reduced pressure and the residue was washed with dioxane. The crude mixture was purified by Sephadex column chromatography (100% MeOH), affording the title compound. LCMS Method B, RT=3.088 min, m/z=684.3 [M+H]⁺, LCMS Method D, RT=3.639 min, m/z=684.2916 [M+H]⁺, exact mass: 683.2874. ¹H NMR (500 MHz, MeOD-d4) δ ppm 8.01 (s, 1H), 7.91 (d, J=8.4 Hz, 1H), 7.75 (d, J=7.5 Hz, 1H), 7.65 (dd, J=7.3, 1.4 Hz, 1H), 7.61 (dd, J=8.2, 7.4 Hz, 1H), 7.53 (dd, J=7.6, 7.4 Hz, 1H), 7.50 (dd, J=7.5, 1.4 Hz, 1H), 7.28 (d, J=6.6 Hz, 1H), 7.26 (d, J=6.4 Hz, 1H), 7.08 (s, 2H), 4.17 (s, 2H), 4.10-4.04 (m, 1H), 4.03 (s, 3H), 3.95 (s, 6H), 3.90 (s, 2H), 3.73-3.63 (m, 4H), 3.63 (s, 2H), 2.81-2.77 (m, 1H), 2.47-2.42 (m, 2H), 1.59-1.54 (m, 2H), 1.46 (s, 3H).

### Example 69: 2-(4-(4-(2-chloro-3-(5-((((cis-3-hydroxycyclobutyl)methyl)amino)methyl)-6-methoxypyridin-2-yl)phenyl)-1H-indazol-1-yl)-2,6-dimethoxybenzyl)-2,6-diazaspiro[3.4]octan-7-one

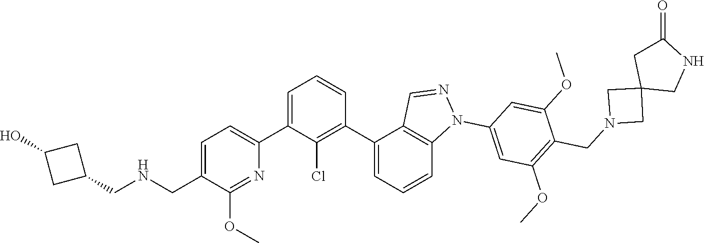 [SMILES: COc1cc(-n2ncc3c(-c4cccc(-c5ccc(CNC[C@H]6C[C@@H](O)C6)c(OC)n5)c4Cl)cccc32)cc(OC)c1CN1CC2(CNC(=O)C2)C1]

A mixture of Intermediate E4 (50 mg, 0.07 mmol) and 2,6-diazaspiro[3.4]octan-7-one (10.09 mg, 0.08 mmol) in 6 mL MeOH/DCM (1:1) was stirred at room temperature and acetic acid was added until the pH was around 4. After the reaction was stirred for 1 hour, sodium cyanoborohydride (21.99 mg, 0.35 mmol) was added and the reaction was stirred at room temperature for an additional 1 hour. The crude mixture was further used without purification.

The residue was added with 4M HCl in dioxane (1 mL) and stirred at room temperature for 30 minutes. The residual solvent was removed under reduced pressure and the residue was washed with dioxane. The crude mixture was purified by Sephadex column chromatography (100% MeOH), affording the title compound. LCMS Method B, RT=3.037 min, m/z=723.4 [M+H]⁺, LCMS Method D, RT=3.580 min, m/z=723.3075 [M+H]⁺, exact mass: 722.2983.

Example 70: (S)-1-((6-(2-chloro-3-(1-(3,5-dimethoxy-4-((((5-oxopyrrolidin-2-yl)methyl)amino)methyl)phenyl)-1H-pyrrolo[2,3-b]pyridin-4-yl)phenyl)-2-methoxypyridin-3-yl)methyl)-3-methylazetidine-3-carboxylic acid

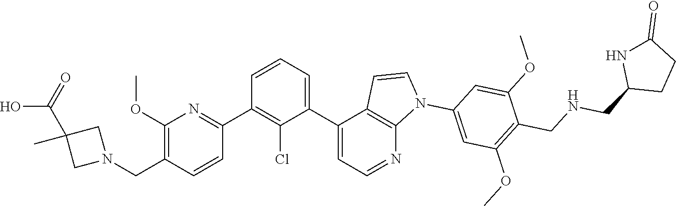 [SMILES: COc1cc(-n2ccc3c(-c4cccc(-c5ccc(CN6CC(C)(C(=O)O)C6)c(OC)n5)c4Cl)ccnc32)cc(OC)c1CNC[C@@H]1CCC(=O)N1]

Step 1: 1-((6-(2-chloro-3-(1-(4-formyl-3,5-dimethoxyphenyl)-1H-pyrrolo[2,3-b]pyridin-4-yl)phenyl)-2-methoxypyridin-3-yl)methyl)-3-methylazetidine-3-carboxylic acid

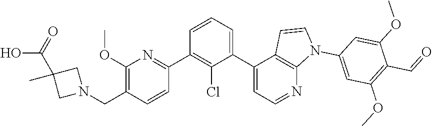 [SMILES: COc1cc(-n2ccc3c(-c4cccc(-c5ccc(CN6CC(C)(C(=O)O)C6)c(OC)n5)c4Cl)ccnc32)cc(OC)c1C=O]

A mixture of Intermediate C1-3 (110.0 mg, 0.26 mmol), Intermediate B3-1 (126.6 mg, 0.31 mmol), Pd(PPh₃)₄ (60.0 mg, 0.05 mmol) and K₂CO₃ (86.2 mg, 0.62 mmol) in a mixture of 1,4-dioxane and H₂O (6:1) 14 mL was sparged with argon for 10 min before stirring at 95° C. for 3 h under argon atmosphere. Then, the mixture was cooled down and diluted with brine. The resulting mixture was extracted 3 times with EtOAc and the organic layer was collected, dried over anhydrous Na₂SO₄, filtered, and evaporated to dryness. The crude mixture was purified by column chromatography (silica gel, gradient elution, 0 to 20% MeOH/DCM) to provide the title compound (10.6 mg). LCMS Method C, RT=4.541 min, m/z=625.2 [M−H]⁺, exact mass: 626.1932.

Step 2: (S)-1-((6-(2-chloro-3-(1-(3,5-dimethoxy-4-((((5-oxopyrrolidin-2-yl)methyl)amino)methyl)phenyl)-1H-pyrrolo[2,3-b]pyridin-4-yl)phenyl)-2-methoxypyridin-3-yl)methyl)-3-methylazetidine-3-carboxylic acid

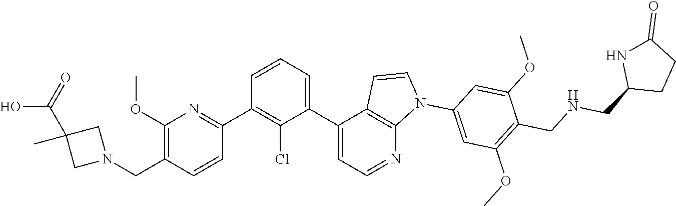 [SMILES: COc1cc(-n2ccc3c(-c4cccc(-c5ccc(CN6CC(C)(C(=O)O)C6)c(OC)n5)c4Cl)ccnc32)cc(OC)c1CNC[C@@H]1CCC(=O)N1]

A mixture of 1-((6-(2-chloro-3-(1-(4-formyl-3,5-dimethoxyphenyl)-1H-pyrrolo[2,3-b]pyridin-4-yl)phenyl)-2-methoxypyridin-3-yl)methyl)-3-methylazetidine-3-carboxylic acid (10.6 mg, 0.02 mmol) and (S)-5-(aminomethyl)pyrrolidin-2-one (5.8 mg, 0.05 mmol) in 4 mL MeOH/DCM (1:1) was stirred at room temperature and added with acetic acid until the pH was around 4. After the reaction was stirred for 1 hour, sodium cyanoborohydride (12.8 mg, 0.19 mmol) was added and the reaction was stirred at room temperature for overnight. The reaction mixture was purified by column chromatography (silica gel, gradient elution, 0 to 50% MeOH/DCM). The fractions containing the product were collected and the solvent was removed under reduced pressure, resulting the title compound. LCMS Method C, RT=4.015 min, m/z=725.3 [M+H]⁺, LCMS Method D, RT=3.569 min, m/z=725.2855 [M+H]⁺, exact mass: 724.2776.

### Example 71: 1-(4-(4-(2-chloro-3-(5-(4,5-dihydro-1H-imidazol-2-yl)-6-methoxypyridin-2-yl)phenyl)-1H-indazol-1-yl)-2,6-dimethoxybenzyl)-3-methylazetidine-3-carboxylic acid

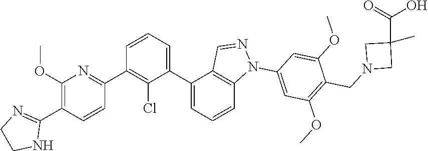 [SMILES: COc1cc(-n2ncc3c(-c4cccc(-c5ccc(C6=NCCN6)c(OC)n5)c4Cl)cccc32)cc(OC)c1CN1CC(C)(C(=O)O)C1]

NEt₃ was added to a mixture of 3-methylazetidine-3-carboxylic acid hydrochloride (20.5 mg, 0.14 mmol) in 6 mL DMF/DCM (1:2) until the pH was around 7. Then, the mixture was transferred to Intermediate E5 (36.0 mg, 0.06 mmol) at room temperature. After that, acetic acid was added until the pH was around 4. After the reaction was stirred for 1 hour, sodium cyanoborohydride (22.0 mg, 0.32 mmol) was added and the reaction was stirred at room temperature for overnight. The reaction mixture was purified by column chromatography (silica gel, gradient elution, 0 to 40% MeOH/DCM). The fractions containing the product were collected and the solvent was removed under reduced pressure to provide the title compound. LCMS Method B, RT=3.172 min, m/z=667.3 [M+H]⁺, LCMS Method D, RT=3.690 min, m/z=667.2436 [M+H]⁺, exact mass: 666.2357.

### Example 72: 1-(4-(4-(2-chloro-3-(5-(4,5-dihydro-1H-imidazol-2-yl)-6-methoxypyridin-2-yl)phenyl)-1H-indazol-1-yl)-2,6-dimethoxybenzyl)-3-methylazetidin-3-ol

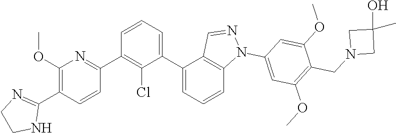 [SMILES: COc1cc(-n2ncc3c(-c4cccc(-c5ccc(C6=NCCN6)c(OC)n5)c4Cl)cccc32)cc(OC)c1CN1CC(C)(O)C1]

A mixture of Intermediate E5 (20.5 mg, 0.04 mmol) and 3-methylazetidin-3-ol hydrochloride (10.5 mg, 0.08 mmol) in 4 mL DMF/DCM (1:3) was stirred at room temperature and added with acetic acid until the pH was around 4. After the reaction was stirred for 1 hour, sodium cyanoborohydride (13.2 mg, 0.19 mmol) was added and the reaction was stirred at room temperature for 2 hours. The crude mixture was purified by column chromatography (silica gel, gradient elution, 0 to 40% MeOH/DCM). The fractions containing the product were collected and the solvent was removed under reduced pressure to provide the title compound. LCMS Method B, RT=3.117 min, m/z=639.3 [M+H]⁺, LCMS Method D, RT=3.681 min, m/z=639.2502 [M+H]⁺, exact mass: 638.2408.

### Example 73: (S)-5-(((4-(7-(2-chloro-3-(5-((3-hydroxy-3-methylazetidin-1-yl)methyl)-6-methoxypyridin-2-yl)phenyl)-1-methyl-1H-indazol-3-yl)-2,6-dimethoxybenzyl)amino)methyl)pyrrolidin-2-one

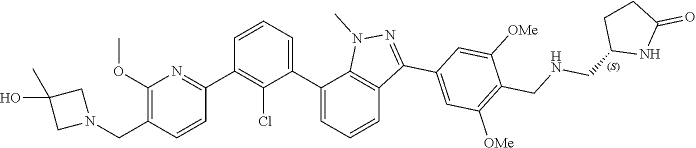

### Step 1: 7-(4,4,5,5-tetramethyl-1,3,2-dioxaborolan-2-yl)-1H-indazole

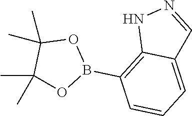 [SMILES: CC1(C)OB(c2cccc3cn[nH]c23)OC1(C)C]

Pd(dppf)Cl₂·CH₂Cl₂ (82.0 mg, 0.1 mmol) was added to a solution of 7-bromo-1H-indazole (0.40 g, 2.0 mmol), bis(pinacolato)diboron (600 mg, 2.5 mmol), and KOAc (1.4 g, 14 mmol) in DMF 7 mL. The resulting suspension was sparged with argon for 10 min. After the reaction was sealed and stirred at 150° C. for 1 hour under argon atmosphere, it was cooled down and diluted with water. The resulting mixture was extracted 3 times with EtOAc and the organic layer was collected, dried over anhydrous Na₂SO₄, filtered, and evaporated to dryness. The product was further used without purification.

### Step 2: 1-((6-(2-chloro-3-(1H-indazol-7-yl)phenyl)-2-methoxypyridin-3-yl)methyl)-3-methylazetidin-3-ol

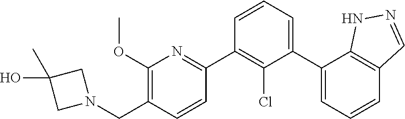 [SMILES: COc1nc(-c2cccc(-c3cccc4cn[nH]c34)c2Cl)ccc1CN1CC(C)(O)C1]

A mixture of 7-(4,4,5,5-tetramethyl-1,3,2-dioxaborolan-2-yl)-1H-indazole (0.1 mmol of crude mixture), Intermediate C1-2 (510 mg, 1.0 mmol), Pd(PPh₃)₄ (115 mg, 0.1 mmol) and K₂CO₃ (415 mg, 3.0 mmol) in a mixture of 1,4-dioxane and H₂O (6:1) 15 mL was sparged with argon for 10 min before it was stirred at 95° C. for 60 min under argon atmosphere. Then, the mixture was cooled down and diluted with brine. The resulting mixture was extracted 3 times with EtOAc and the organic layer was collected, dried over anhydrous Na₂SO₄, filtered, and evaporated to dryness. The crude mixture was purified by column chromatography (silica gel, gradient elution, 0 to 15% MeOH/DCM) to provide the product.

### Step 3: 1-((6-(2-chloro-3-(3-iodo-1H-indazol-7-yl)phenyl)-2-methoxypyridin-3-yl)methyl)-3-methylazetidin-3-ol

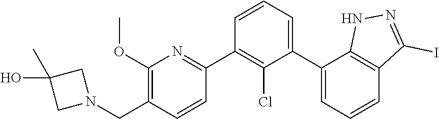 [SMILES: COc1nc(-c2cccc(-c3cccc4c(I)n[nH]c34)c2Cl)ccc1CN1CC(C)(O)C1]

Potassium hydroxide (35 mg, 0.63 mmol) was added to a solution 1-((6-(2-chloro-3-(3-iodo-1H-indazol-7-yl)phenyl)-2-methoxypyridin-3-yl)methyl)-3-methylazetidin-3-ol (109 mg, 0.25 mmol) in DMF (3 mL) After the reaction was stirred at 0° C. for 1 hour, iodine (66 mg, 0.26 mmol) was added and stirred for 2.5 h. Then, the reaction was quenched with sodium thiosulfate. The resulting mixture was extracted 3 times with EtOAc and the organic layer was collected, dried over anhydrous Na₂SO₄, filtered, and evaporated to dryness to provide the product. The product was further used without purification.

### Step 4: 1-((6-(2-chloro-3-(3-iodo-1-methyl-1H-indazol-7-yl)phenyl)-2-methoxypyridin-3-yl)methyl)-3-methylazetidin-3-ol

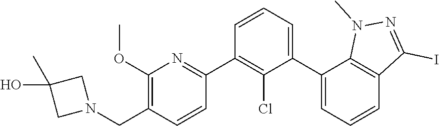 [SMILES: COc1nc(-c2cccc(-c3cccc4c(I)nn(C)c34)c2Cl)ccc1CN1CC(C)(O)C1]

NaH (12 mg, 0.50 mmol) was added into a solution of 1-((6-(2-chloro-3-(3-iodo-1H-indazol-7-yl)phenyl)-2-methoxypyridin-3-yl)methyl)-3-methylazetidin-3-ol (0.25 mmol) in DMF (2 mL). After stirring at room temperature for 1 hour, the solution was cooled down in an ice bath. Methyl iodide (0.02 mL, 0.26 mmol) was added into the solution and the reaction was stirred for 60 min. The reaction was quenched by water and extracted with EtOAc. The organic layer was collected, dried over anhydrous Na₂SO₄, filtered, and evaporated to dryness. The product was further used without purification. The crude mixture was purified by column chromatography (silica gel, gradient elution, 0 to 20% MeOH/DCM) to provide the product.

### Step 5: 4-(7-(2-chloro-3-(5-((3-hydroxy-3-methylazetidin-1-yl)methyl)-6-methoxypyridin-2-yl)phenyl)-1-methyl-1H-indazol-3-yl)-2,6-dimethoxybenzaldehyde

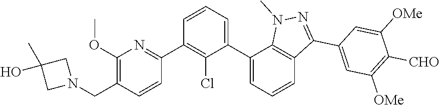 [SMILES: COc1cc(-c2nn(C)c3c(-c4cccc(-c5ccc(CN6CC(C)(O)C6)c(OC)n5)c4Cl)cccc23)cc(OC)c1C=O]

A mixture of 1-((6-(2-chloro-3-(3-iodo-1-methyl-1H-indazol-7-yl)phenyl)-2-methoxypyridin-3-yl)methyl)-3-methylazetidin-3-ol (103 mg, 0.18 mmol), 2,6-dimethoxy-4-(4,4,5,5-tetramethyl-1,3,2-dioxaborolan-2-yl)benzaldehyde (0.30 mmol), Pd(PPh₃)₄ (21 mg, 0.018 mmol) and K₂CO₃ (74 mg, 0.54 mmol) in a mixture of 1,4-dioxane and H₂O (6:1) 10 mL was sparged with argon for 10 min before it was stirred at 75° C. for 30 min under argon atmosphere. Then, the mixture was cooled down and diluted with brine. The resulting mixture was extracted 3 times with EtOAc and the organic layer was collected, dried over anhydrous Na₂SO₄, filtered, and evaporated to dryness. The crude mixture was purified by column chromatography (silica gel, gradient elution, 0 to 20% MeOH/DCM) to provide the product.

### Step 6: (S)-5-(((4-(7-(2-chloro-3-(5-((3-hydroxy-3-methylazetidin-1-yl)methyl)-6-methoxypyridin-2-yl)phenyl)-1-methyl-1H-indazol-3-yl)-2,6-dimethoxybenzyl)amino)methyl)pyrrolidin-2-one

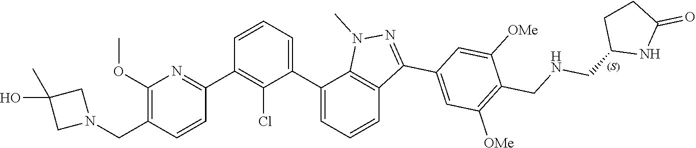

A mixture 4-(7-(2-chloro-3-(5-((3-hydroxy-3-methylazetidin-1-yl)methyl)-6-methoxypyridin-2-yl)phenyl)-1-methyl-1H-indazol-3-yl)-2,6-dimethoxybenzaldehyde (61 mg, 0.1 mmol) and (S)-5-(aminomethyl)pyrrolidin-2-one (12 mg, 0.105 mmol) in 4 mL MeOH/DCM (1:1) was stirred at room temperature and added with acetic acid until the pH value around 4. After 30 min, sodium cyanoborohydride (19 mg, 0.30 mmol) was added and the reaction was stirred at room temperature for an additional 1 hour. The crude mixture was purified by Sephadex column chromatography (100% MeOH) to provide the title compound. LCMS Method B, RT=3.059 min, m/z=711.2 [M+H]⁺, LCMS Method D, RT=3.618 min, m/z=711.3007 [M+H]⁺, exact mass: 710.2984.

### Example 74: (S)-5-(((4-(4-(2-chloro-3-(5-(4,5-dihydro-1H-imidazol-2-yl)-6-methoxypyridin-2-yl)phenyl)-1H-indazol-1-yl)-2,6-dimethoxybenzyl)amino)methyl)pyrrolidin-2-one

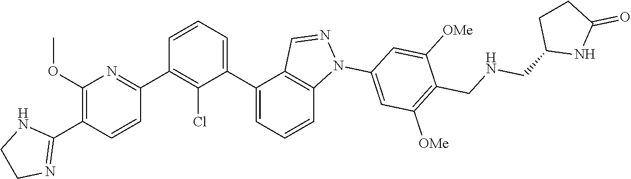 [SMILES: COc1cc(-n2ncc3c(-c4cccc(-c5ccc(C6=NCCN6)c(OC)n5)c4Cl)cccc32)cc(OC)c1CNC[C@@H]1CCC(=O)N1]

A mixture of Intermediate E5 (34.1 mg, 0.06 mmol) and (S)-5-(aminomethyl)pyrrolidin-2-one (7.2 mg, 0.063 mmol) in 4 mL DMF/DCM (1:3) was stirred at room temperature and added with acetic acid until the pH was around 4. After leaving for 30 min, sodium cyanoborohydride (11.4 mg, 0.18 mmol) was added and the reaction was stirred at room temperature for 2 hours. The crude mixture was purified by Sephadex column chromatography (100% MeOH). The fractions containing the product were collected and the solvent was removed under reduced pressure to provide the title compound. LCMS Method B, RT=3.038 min, m/z=666.2 [M+H]⁺, LCMS Method D, RT=3.559 min, m/z=666.2576 [M+H]⁺, exact mass: 665.2517.

### Example 75: (S)-5-(((4-(7-(2-chloro-3-(5-((((cis-3-hydroxycyclobutyl)methyl)amino)methyl)-6-methoxypyridin-2-yl)phenyl)-1-methyl-1H-indazol-3-yl)-2,6-dimethoxybenzyl)amino)methyl)pyrrolidin-2-one

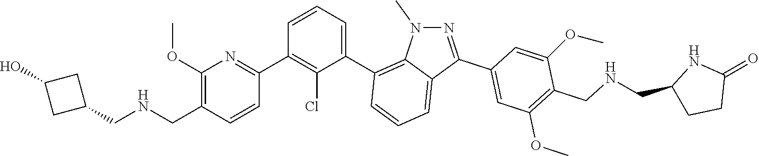 [SMILES: COc1cc(-c2nn(C)c3c(-c4cccc(-c5ccc(CNC[C@H]6C[C@@H](O)C6)c(OC)n5)c4Cl)cccc23)cc(OC)c1CNC[C@@H]1CCC(=O)N1]

### Step 1: tert-butyl ((6-(2-chloro-3-(1H-indazol-7-yl)phenyl)-2-methoxypyridin-3-yl)methyl)((cis-3-hydroxycyclobutyl)methyl)carbamate

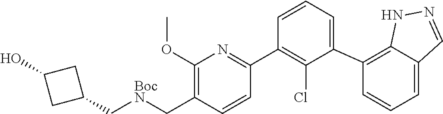 [SMILES: COc1nc(-c2cccc(-c3cccc4cn[nH]c34)c2Cl)ccc1CN(C[C@H]1C[C@@H](O)C1)C(=O)OC(C)(C)C]

A mixture of 7-(4,4,5,5-tetramethyl-1,3,2-dioxaborolan-2-yl)-1H-indazole (0.7 mmol of crude mixture, prepared by using similar procedures described in Step 1 of Example 73), Intermediate C1-5 (510 mg, 1.0 mmol), Pd(PPh₃)₄ (81 mg, 0.07 mmol) and K₂CO₃ (300 mg, 2.1 mmol) in a mixture of 1,4-dioxane and H₂O (6:1) 15 mL, was sparged with argon for 10 min before it was stirred at 95° C. for 30 min under argon atmosphere. Then, the mixture was cooled down and diluted with brine. The resulting mixture was extracted 3 times with EtOAc and the organic layer was collected, dried over anhydrous Na₂SO₄, filtered, and evaporated to dryness. The crude mixture was purified by column chromatography (silica gel, gradient elution, 0 to 10% MeOH/DCM) to provide the product.

### Step 2: tert-butyl ((6-(2-chloro-3-(3-iodo-1H-indazol-7-yl)phenyl)-2-methoxypyridin-3-yl)methyl)((cis-3-hydroxycyclobutyl)methyl)carbamate

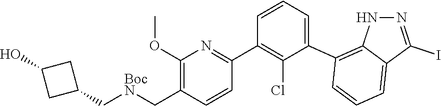 [SMILES: COc1nc(-c2cccc(-c3cccc4c(I)n[nH]c34)c2Cl)ccc1CN(C[C@H]1C[C@@H](O)C1)C(=O)OC(C)(C)C]

Potassium hydroxide (0.02 g, 0.35 mmol) was added to a solution of tert-butyl ((6-(2-chloro-3-(1H-indazol-7-yl)phenyl)-2-methoxypyridin-3-yl)methyl)((cis-3-hydroxycyclobutyl)methyl)carbamate (80 mg, 0.14 mmol) in DMF (3 mL). After the reaction was stirred at 0° C. for 1 hour, iodine (37 mg, 0.15 mmol) was added and stirred for 2 h. Then, the reaction was quenched with sodium thiosulfate. The resulting mixture was extracted 3 times with EtOAc and the organic layer was collected, dried over anhydrous Na₂SO₄, filtered, and evaporated to dryness to provide the product. The product was further used without purification.

### Step 3: tert-butyl ((6-(2-chloro-3-(3-iodo-1-methyl-1H-indazol-7-yl)phenyl)-2-methoxypyridin-3-yl)methyl)((cis-3-hydroxycyclobutyl)methyl)carbamate

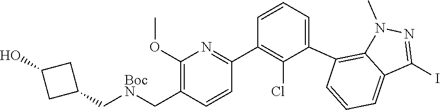 [SMILES: COc1nc(-c2cccc(-c3cccc4c(I)nn(C)c34)c2Cl)ccc1CN(C[C@H]1C[C@@H](O)C1)C(=O)OC(C)(C)C]

NaH (32 mg, 0.65 mmol) was added into a solution of tert-butyl ((6-(2-chloro-3-(3-iodo-1H-indazol-7-yl)phenyl)-2-methoxypyridin-3-yl)methyl)((cis-3-hydroxycyclobutyl)methyl)carbamate (0.24 mmol) in DMF (4 mL). After stirring at room temperature for 1 hour, the solution was cooled down in an ice bath. Methyl iodide (0.04 mL, 0.32 mmol) was added into the solution and the reaction was stirred for 30 min. The reaction was quenched by water and extracted with EtOAc. The organic layer was collected, dried over anhydrous Na₂SO₄, filtered, and evaporated to dryness. The product was further used without purification. The crude mixture was purified by column chromatography (silica gel, gradient elution, 0 to 10% MeOH/DCM) to provide the product.

### Step 4: tert-butyl ((6-(2-chloro-3-(3-(4-formyl-3,5-dimethoxyphenyl)-1-methyl-1H-indazol-7-yl)phenyl)-2-methoxypyridin-3-yl)methyl)((cis-3-hydroxycyclobutyl)methyl)carbamate

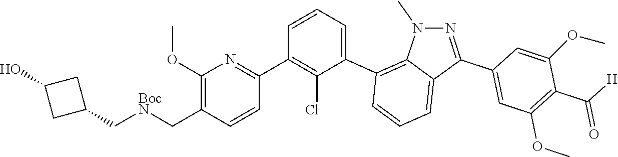 [SMILES: COc1cc(-c2nn(C)c3c(-c4cccc(-c5ccc(CN(C[C@H]6C[C@@H](O)C6)C(=O)OC(C)(C)C)c(OC)n5)c4Cl)cccc23)cc(OC)c1C=O]

A mixture of tert-butyl ((6-(2-chloro-3-(3-iodo-1-methyl-1H-indazol-7-yl)phenyl)-2-methoxypyridin-3-yl)methyl)((cis-3-hydroxycyclobutyl)methyl)carbamate (73 mg, 0.1 mmol), 2,6-dimethoxy-4-(4,4,5,5-tetramethyl-1,3,2-dioxaborolan-2-yl)benzaldehyde (0.2 mmol), Pd(PPh₃)₄ (2.3 mg, 0.002 mmol) and K₂CO₃ (10 mg, 0.07 mmol) in a mixture of 1,4-dioxane and H₂O (6:1) 10 mL was sparged with argon for 10 min before it was stirred at 75° C. for 30 min under argon atmosphere. Then, the mixture was cooled down and diluted with brine. The resulting mixture was extracted 3 times with EtOAc and the organic layer was collected, dried over anhydrous Na₂SO₄, filtered, and evaporated to dryness. The crude mixture was purified by column chromatography (silica gel, gradient elution, 0 to 10% MeOH/EtOAc) to provide the product.

### Step 5: (S)-5-(((4-(7-(2-chloro-3-(5-((((cis-3-hydroxycyclobutyl)methyl)amino)methyl)-6-methoxypyridin-2-yl)phenyl)-1-methyl-1H-indazol-3-yl)-2,6-dimethoxybenzyl)amino)methyl)pyrrolidin-2-one

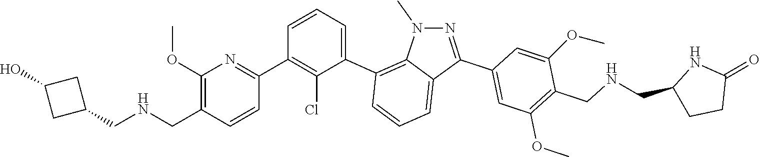 [SMILES: COc1cc(-c2nn(C)c3c(-c4cccc(-c5ccc(CNC[C@H]6C[C@@H](O)C6)c(OC)n5)c4Cl)cccc23)cc(OC)c1CNC[C@@H]1CCC(=O)N1]

A mixture tert-butyl ((6-(2-chloro-3-(3-(4-formyl-3,5-dimethoxyphenyl)-1-methyl-1H-indazol-7-yl)phenyl)-2-methoxypyridin-3-yl)methyl((cis-3-hydroxycyclobutyl)methyl)carbamate (80 mg, 0.11 mmol) and (S)-5-(aminomethyl)pyrrolidin-2-one (18 mg, 0.15 mmol) in 6 mL MeOH/DCM (1:1) was acidified with acetic acid (pH around 4). After the reaction was stirred for 1 hour, sodium cyanoborohydride (63 mg, 0.55 mmol) was added and the reaction was stirred at room temperature for an additional 1 hour. The crude mixture was purified by column chromatography (silica gel, gradient elution, 10 to 15% MeOH/DCM). After evaporation to dryness, the resulting product was stirred at room temperature in the presence of 4M HCl in dioxane for 30 min. All solvent was evaporated under vacuum and the crude mixture was purified by column chromatography (silica gel, gradient elution, to 10% MeOH/DCM). The fractions containing the product were collected and the solvent was removed under reduced pressure, to provide the title compound. LCMS Method B, RT=3.065 min, m/z=725.4 [M+H]⁺, LCMS Method D, RT=3.623 min, m/z=725.3230 [M+H]⁺, exact mass: 724.3100.

### Example 76: 2-(4-(4-(2-chloro-3-(5-(4,5-dihydro-1H-imidazol-2-yl)-6-methoxypyridin-2-yl)phenyl)-1H-indazol-1-yl)-2,6-dimethoxybenzyl)-2,6-diazaspiro[3.4]octan-7-one

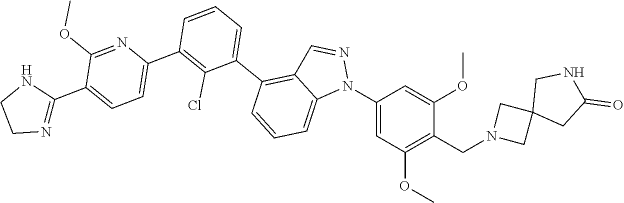 [SMILES: COc1cc(-n2ncc3c(-c4cccc(-c5ccc(C6=NCCN6)c(OC)n5)c4Cl)cccc32)cc(OC)c1CN1CC2(CNC(=O)C2)C1]

NEt₃ was added to a mixture of 2,6-diazaspiro[3.4]octan-7-one hydrochloride (12 mg, 0.07 mmol) in 5 mL DMF/DCM (1:2) until the pH was around 7. Then, the mixture was transferred to Intermediate E5 (36.0 mg, 0.06 mmol) at room temperature. After that, acetic acid was added until the pH was around 4. After the reaction was stirred for 1 hour, sodium cyanoborohydride (22.0 mg, 0.32 mmol) was added and the reaction was stirred at room temperature for overnight. The reaction mixture was purified by Sephadex column chromatography (100% MeOH). The fractions containing the product were collected and the solvent was removed under reduced pressure to provide the title compound. LCMS Method B, RT=3.066 min, m/z=678.3 [M+H]⁺, exact mass: 677.2517.

### Example 77: cis-3-(((4-(4-(2-chloro-3-(5-(4,5-dihydro-1H-imidazol-2-yl)-6-methoxypyridin-2-yl)phenyl)-1H-indazol-1-yl)-2,6-dimethoxybenzyl)amino)methyl)cyclobutanol

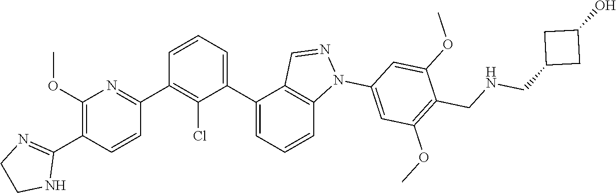 [SMILES: COc1cc(-n2ncc3c(-c4cccc(-c5ccc(C6=NCCN6)c(OC)n5)c4Cl)cccc32)cc(OC)c1CNC[C@H]1C[C@@H](O)C1]

A mixture of Intermediate E5 (20.0 mg, 0.04 mmol) and cis-3-(aminomethyl)cyclobutanol (10.0 mg, 0.10 mmol) in 6 mL DMF/DCM (1:1) was stirred at room temperature and added with acetic acid until the pH was around 4. After the reaction was stirred for 1 hour, sodium cyanoborohydride (12.3 mg, 0.18 mmol) was added and the reaction was stirred at room temperature for 2 hours. The crude mixture was purified by Sephadex column chromatography (100% MeOH) to provide the title compound. LCMS Method B, RT=3.088 min, m/z=653.3 [M+H]⁺, LCMS Method D, RT=3.654 min, m/z=653.2623 [M+H]⁺, LCMS Method D, RT=3.557 min, m/z=678.2580 [M+H]⁺, exact mass: 677.2517.

### Example 78: (S)-5-((((6-(2-chloro-3-(1-(4-((((cis-3-hydroxycyclobutyl)methyl)amino)methyl)-3,5-dimethoxyphenyl)-1H-indazol-4-yl)phenyl)-2-methoxypyridin-3-yl)methyl)amino)methyl)pyrrolidin-2-one

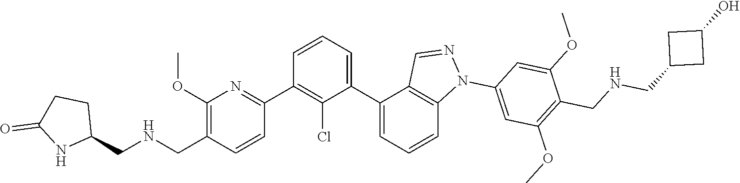 [SMILES: COc1cc(-n2ncc3c(-c4cccc(-c5ccc(CNC[C@@H]6CCC(=O)N6)c(OC)n5)c4Cl)cccc32)cc(OC)c1CNC[C@H]1C[C@@H](O)C1]

### Step 1: cis-3-(((4-(4-bromo-1H-indazol-1-yl)-2,6-dimethoxybenzyl)amino) methyl)cyclobutanol

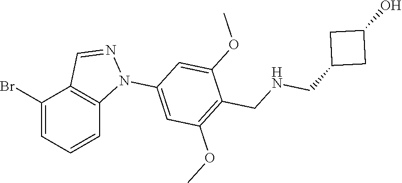 [SMILES: COc1cc(-n2ncc3c(Br)cccc32)cc(OC)c1CNC[C@H]1C[C@@H](O)C1]

A mixture of Intermediate B4 (0.07 g, 0.19 mmol) and cis-3-(aminomethyl)cyclobutanol (0.04 g, 0.40 mmol) in 8 mL MeOH/DCM (1:1) was stirred at room temperature and added with acetic acid until the pH was around 4. After the reaction was stirred for 1 hour, sodium cyanoborohydride (0.06 g, 0.87 mmol) was added and the reaction was stirred at room temperature for overnight. The reaction mixture was purified by column chromatography (silica gel, gradient elution, 0 to 30% MeOH/DCM). The fractions containing the product were collected and the solvent was removed under reduced pressure to provide cis-3-(((4-(4-bromo-1H-indazol-1-yl)-2,6-dimethoxybenzyl)amino)methyl)cyclobutanol (0.09 g). LCMS Method B, RT=3.370 min, m/z=446.1 [M+H]⁺, exact mass: 445.1001.

### Step 2: 6-(2-chloro-3-(1-(4-((((cis-3-hydroxycyclobutyl)methyl)amino)methyl)-3,5-dimethoxyphenyl)-1H-indazol-4-yl)phenyl)-2-methoxynicotinaldehyde

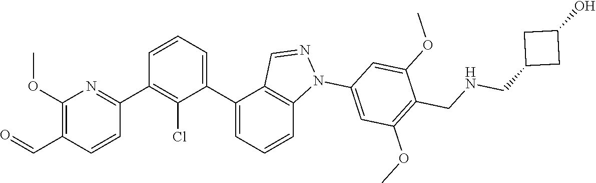 [SMILES: COc1cc(-n2ncc3c(-c4cccc(-c5ccc(C=O)c(OC)n5)c4Cl)cccc32)cc(OC)c1CNC[C@H]1C[C@@H](O)C1]

A mixture of Intermediate C2 (0.14 g, 0.37 mmol), cis-3-(((4-(4-bromo-1H-indazol-1-yl)-2,6-dimethoxybenzyl)amino) methyl)cyclobutanol (0.09 g, 0.20 mmol), Pd(PPh₇)₄ (0.02 g, 0.02 mmol) and K₂CO₃ (0.08 g, 0.58 mmol) in a mixture of 1,4-dioxane and H₂O (6:1) 10 mL was sparged with argon for 10 min before it was stirred at 95° C. for 2 hours under argon atmosphere. Then, the mixture was cooled down and diluted with brine. The resulting mixture was extracted 3 times with EtOAc and the organic layer was collected, dried over anhydrous Na₂SO₄, filtered, and evaporated to dryness. The crude mixture was purified by column chromatography (silica gel, gradient elution, 0 to 30% MeOH/DCM) to provide 6-(2-chloro-3-(1-(4-((((cis-3-hydroxycyclobutyl)methyl)amino)methyl)-3,5-dimethoxyphenyl)-1H-indazol-4-yl)phenyl)-2-methoxynicotinaldehyde (0.07 g). LCMS Method B, RT=3.906 min, m/z=613.3 [M+H]⁺, exact mass: 612.2139.

### Step 3: (S)-5-((((6-(2-chloro-3-(1-(4-((((cis-3-hydroxycyclobutyl)methyl)amino)methyl)-3,5-dimethoxyphenyl)-1H-indazol-4-yl)phenyl)-2-methoxypyridin-3-yl)methyl)amino)methyl)pyrrolidin-2-one

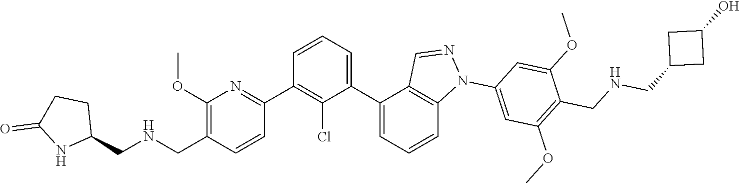 [SMILES: COc1cc(-n2ncc3c(-c4cccc(-c5ccc(CNC[C@@H]6CCC(=O)N6)c(OC)n5)c4Cl)cccc32)cc(OC)c1CNC[C@H]1C[C@@H](O)C1]

A mixture of 6-(2-chloro-3-(1-(4-((((cis-3-hydroxycyclobutyl)methyl)amino)methyl)-3,5-dimethoxyphenyl)-1H-indazol-4-yl)phenyl)-2-methoxynicotinaldehyde (0.07 g, 0.11 mmol) and (S)-5-(aminomethyl)pyrrolidin-2-one (0.02 g, 0.18 mmol) in 4 mL MeOH/DCM (1:1) was stirred at room temperature and added with acetic acid until the pH was around 4. After the reaction was stirred for 1 hour, sodium cyanoborohydride (0.04 g, 0.58 mmol) was added and the reaction was stirred at room temperature for 2 hours. The crude mixture was purified by column chromatography (silica gel, gradient elution, 0 to 40% MeOH/DCM). The fractions containing the product were collected and the solvent was removed under reduced pressure to provide the title compound. LCMS Method B, RT=3.030 min, m/z=711.4 [M+H]⁺, LCMS Method D, RT=3.574 min, m/z=711.3049 [M+H]⁺, exact mass: 710.2983.

### Example 79: 2-((6-(2-chloro-3-(1-(4-((3-hydroxy-3-methylazetidin-1-yl)methyl)-3,5-dimethoxyphenyl)-1H-indazol-4-yl)phenyl)-2-methoxypyridin-3-yl)methyl)-2,6-diazaspiro[3.4]octan-7-one

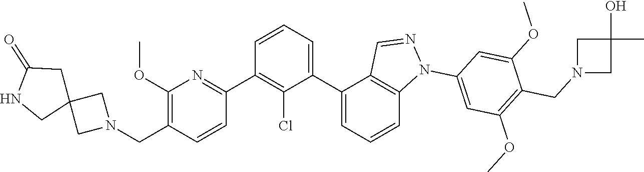 [SMILES: COc1cc(-n2ncc3c(-c4cccc(-c5ccc(CN6CC7(CNC(=O)C7)C6)c(OC)n5)c4Cl)cccc32)cc(OC)c1CN1CC(C)(O)C1]

NEt₃ was added to a mixture of 3-methylazetidin-3-ol hydrochloride (0.04 g, 0.32 mmol) in 3 mL MeOH/DCM (1:1) until the pH was around 7. Then, the mixture was transferred to a mixture of Intermediate E6 (0.12 g, 0.19 mmol) in 5 mL MeOH/DCM (1:1) at room temperature. After that, acetic acid was added until the pH was around 4. After the reaction was stirred for 1 hour, sodium cyanoborohydride (0.05 g, 0.73 mmol) was added and the reaction was stirred at room temperature for 2 hours. The crude mixture was purified by Sephadex column chromatography (100% MeOH). The fractions containing the product were collected and the solvent was removed under reduced pressure, resulting in the title compound. LCMS Method B, RT=3.047 min, m/z=709.3 [M+H]⁺, LCMS Method D, RT=3.590 min, m/z=709.2918 [M+H]⁺ exact mass: 708.2827.

### Example 80: (S)-5-((((6-(4-(2,3-dihydrobenzo[b][1,4]dioxin-6-yl)indolin-1-yl)-2-methoxypyridin-3-yl)methyl)amino)methyl)pyrrolidin-2-one

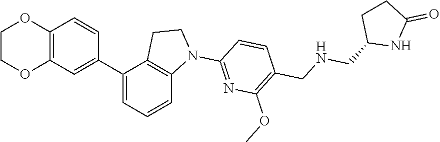 [SMILES: COc1nc(N2CCc3c(-c4ccc5c(c4)OCCO5)cccc32)ccc1CNC[C@@H]1CCC(=O)N1]

### Step 1: 6-(4-(2,3-dihydrobenzo[b][1,4]dioxin-6-yl)indolin-1-yl)-2-methoxynicotinaldehyde

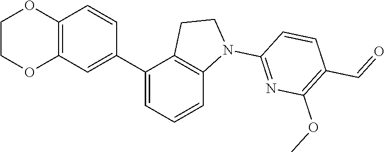 [SMILES: COc1nc(N2CCc3c(-c4ccc5c(c4)OCCO5)cccc32)ccc1C=O]

A mixture of Intermediate A2 (200.00 mg, 0.79 mmol), 6-chloro-2-methoxynicotinaldehyde (162.57 g, 0.95 mmol), Pd₂(dba)₃ (36.17 mg, 0.04 mmol), xantphos (68.57 g, 0.12 mmol) and Cs₂CO₃ (772.20 mg, 2.37 mmol) in toluene 15 mL was sparged with argon for 10 min before it was refluxed under argon atmosphere for overnight. Then, the mixture was cooled down and diluted with water. The resulting mixture was extracted 3 times with EtOAc and the organic layer was collected, dried over anhydrous Na₂SO₄, filtered, and evaporated to dryness. The crude mixture was purified by column chromatography (silica gel, gradient elution, 0 to 60% EtOAc/hexanes) to provide 6-(4-(2,3-dihydrobenzo[b][1,4]dioxin-6-yl)indolin-1-yl)-2-methoxynicotinaldehyde.

### Step 2: (S)-5-((((6-(4-(2,3-dihydrobenzo[b][1,4]dioxin-6-yl)indolin-1-yl)-2-methoxypyridin-3-yl)methyl)amino)methyl)pyrrolidin-2-one

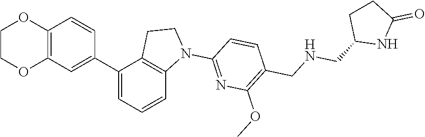 [SMILES: COc1nc(N2CCc3c(-c4ccc5c(c4)OCCO5)cccc32)ccc1CNC[C@@H]1CCC(=O)N1]

A mixture of 6-(4-(2,3-dihydrobenzo[b][1,4]dioxin-6-yl)indolin-1-yl)-2-methoxynicotinaldehyde (100.00 mg, 0.26 mmol) and (S)-5-(aminomethyl)pyrrolidin-2-one (35.22 mg, 0.31 mmol) in 6 mL MeOH/DCM (1:1) was stirred at room temperature and added with acetic acid until the pH was around 4. After the reaction was stirred for 1 hour, sodium cyanoborohydride (81.69 mg, 1.30 mmol) was added and the reaction was stirred at room temperature for an additional 1 hour. The crude mixture was purified by column chromatography (silica gel, gradient elution, 5 to 10% MeOH/DCM). The fractions containing the product were collected and the solvent was removed under reduced pressure, resulting in the title compound as product. LCMS Method C, RT=4.34 min, m/z=487.3 [M+H]⁺, LCMS Method D, RT=4.131 min, m/z=487.2336 [M+H]⁺, exact mass: 486.2267.

Example 81: (S)-5-((((6-(2-chloro-3-(1-(3,5-dimethoxy-4-(((((S)-5-oxopyrrolidin-2-yl)methyl)amino)methyl)phenyl)-1H-pyrrolo[2,3-b]pyridin-4-yl)phenyl)-2-methoxypyridin-3-yl)methyl)amino)methyl)pyrrolidin-2-one

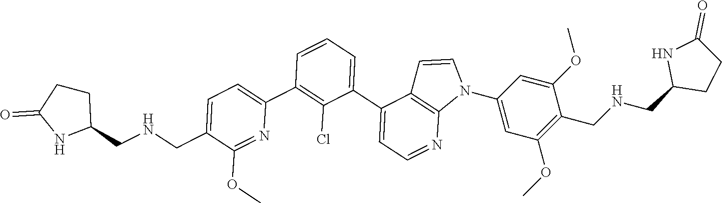 [SMILES: COc1cc(-n2ccc3c(-c4cccc(-c5ccc(CNC[C@@H]6CCC(=O)N6)c(OC)n5)c4Cl)ccnc32)cc(OC)c1CNC[C@@H]1CCC(=O)N1]

### Step 1: 6-(2-chloro-3-(I-(4-formyl-3,5-dimethoxyphenyl)-1H-pyrrolo[2,3-b]pyridin-4-yl)phenyl)-2-methoxynicotinaldehyde

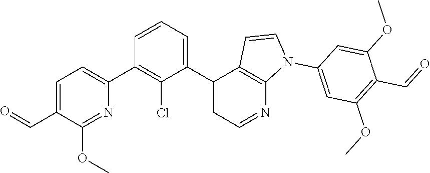 [SMILES: COc1cc(-n2ccc3c(-c4cccc(-c5ccc(C=O)c(OC)n5)c4Cl)ccnc32)cc(OC)c1C=O]

A mixture of Intermediate C1 (200.00 mg, 0.61 mmol), Intermediate B3-1 (300.04 mg, 0.73 mmol), Pd(PPh₃)₄ (70.49 mg, 0.06 mmol) and K₂CO₃ (252.92 mg, 1.83 mmol) in a mixture of 1,4-dioxane and H₂O (6:1) 14 mL was sparged with argon for 10 min before stirring at 95° C. for 1.5 h under argon atmosphere. Then, the mixture was cooled down and diluted with brine. The resulting mixture was extracted 3 times with EtOAc and the organic layer was collected, dried over anhydrous Na₂SO₄, filtered, and evaporated to dryness. The crude mixture was purified by column chromatography (silica gel, gradient elution, 50 to 100% EtOAc/hexanes) to provide 6-(2-chloro-3-(1-(4-formyl-3,5-dimethoxyphenyl)-1H-pyrrolo[2,3-b]pyridin-4-yl)phenyl)-2-methoxynicotinaldehyde.

Step 2: (S)-5-((((6-(2-chloro-3-(1-(3,5-dimethoxy-4-(((((S)-5-oxopyrrolidin-2-yl)methyl)amino)methyl)phenyl)-1H-pyrrolo[2,3-b]pyridin-4-yl)phenyl)-2-methoxypyridin-3-yl)methyl)amino)methyl)pyrrolidin-2-one

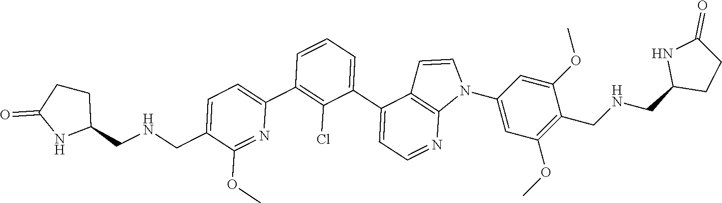 [SMILES: COc1cc(-n2ccc3c(-c4cccc(-c5ccc(CNC[C@@H]6CCC(=O)N6)c(OC)n5)c4Cl)ccnc32)cc(OC)c1CNC[C@@H]1CCC(=O)N1]

A mixture of 6-(2-chloro-3-(1-(4-formyl-3,5-dimethoxyphenyl)-1H-pyrrolo[2,3-b]pyridin-4-yl)phenyl)-2-methoxynicotinaldehyde (60.00 mg, 0.12 mmol) and (S)-5-(aminomethyl)pyrrolidin-2-one (15.55 mg, 0.14 mmol) in 4 mL MeOH/DCM (1:1) was stirred at room temperature and added with acetic acid until the pH was around 4. After the reaction was stirred for 1 hour, sodium cyanoborohydride (37.71 mg, 0.60 mmol) was added and the reaction was stirred at room temperature for an additional 1 hour. The crude mixture was purified by column chromatography (silica gel, gradient elution, 5 to 10% MeOH/DCM). The fractions containing the product were collected and the solvent was removed under reduced pressure, resulting the title compound as product. LCMS Method C, RT=3.87 min, m/z=724.3 [M+H]⁺, 746.3 [M+Na]⁺, LCMS Method D, RT=3.414 min, m/z=724.3001 [M+H]⁺, exact mass: 723.2900.

### Example 82: (S)-5-(((4-(4-(2-chloro-3-(5-((((cis-3-hydroxycyclobutyl)methyl)amino)methyl)-6-methoxypyridin-2-yl)phenyl)-1H-indol-1-yl)-2,6-dimethoxybenzyl)amino)methyl)pyrrolidin-2-one

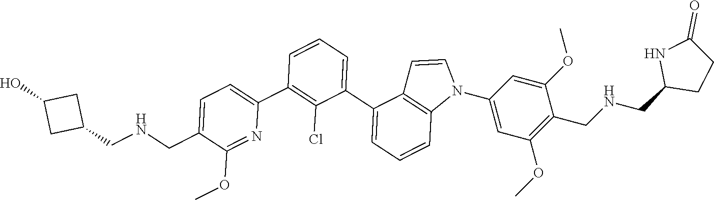 [SMILES: COc1cc(-n2ccc3c(-c4cccc(-c5ccc(CNC[C@H]6C[C@@H](O)C6)c(OC)n5)c4Cl)cccc32)cc(OC)c1CNC[C@@H]1CCC(=O)N1]

A mixture of Intermediate F1 (42.00 mg, 0.06 mmol) and cis-3-(aminomethyl)cyclobutanol (11.72 mg, 0.11 mmol) in 4 mL MeOH/DCM (1:1) was stirred at room temperature and added with acetic acid until the pH was around 4. After the reaction was stirred for 1 hour, sodium cyanoborohydride (18.85 mg, 0.30 mmol) was added. The reaction was stirred at room temperature for an additional 1 hour, quenched by a small amount of MeOH and evaporated to dryness. Then, the residue was dissolved in dioxane and 4M HCl (1 mL) was added. After the reaction was stirred at room temperature for 30 minutes, the residual solvent was removed under reduced pressure and the residue was washed with dioxane. The crude mixture was purified by Sephadex column chromatography (100% MeOH), affording the title compound. LCMS method C, RT=3.971 min, m/z=710.3 [M+H]⁺, LCMS Method D, RT=3.643 min, m/z=710.3113 [M+H]⁺, exact mass: 709.3031.

### Example 83: (S)-5-((((6-(2-chloro-3-(1-(4-(((S)-3-hydroxypyrrolidin-1-yl)methyl)-3,5-dimethoxyphenyl)-1H-indol-4-yl)phenyl)-2-methoxypyridin-3-yl)methyl)amino)methyl)pyrrolidin-2-one

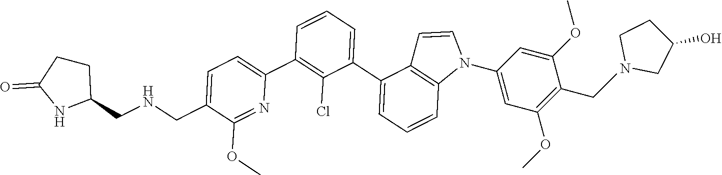

A mixture of Intermediate E1 (58.00 mg, 0.08 mmol) and (S)-pyrrolidin-3-ol hydrochloride (11.72 mg, 0.09 mmol) in 6 mL MeOH/DCM (1:1) was stirred at room temperature and added with acetic acid until the pH was around 4. After the reaction was stirred for 1 hour, sodium cyanoborohydride (25.14 mg, 0.40 mmol) was added. The reaction was stirred at room temperature for an additional 1 hour, quenched by a small amount of MeOH and evaporated to dryness. Then, the residue was dissolved in dioxane and 4M HCl (1 mL) was added. After stirring at room temperature for 30 minutes, the residual solvent was removed under reduced pressure and the residue was washed with dioxane. The crude mixture was purified by Sephadex column chromatography (100% MeOH), affording the title compound. LCMS method B, RT=3.122 min, m/z=696.3 [M+H]⁺, LCMS Method D, RT=3.680 min, m/z=696.2974 [M+H]⁺, exact mass. 695.2900.

### Example 84: 2-(4-(4-(2-chloro-3-(5-((3-hydroxy-3-methylazetidin-1-yl)methyl)-6-methoxypyridin-2-yl)phenyl)-1H-indol-1-yl)-2,6-dimethoxybenzyl)-2,6-diazaspiro[3.4]octan-7-one

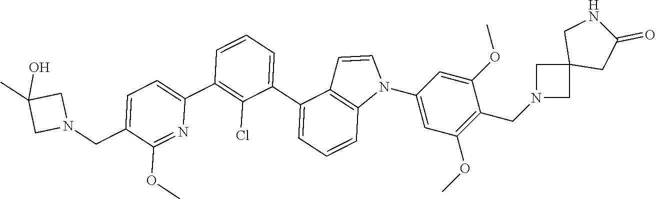 [SMILES: COc1cc(-n2ccc3c(-c4cccc(-c5ccc(CN6CC(C)(O)C6)c(OC)n5)c4Cl)cccc32)cc(OC)c1CN1CC2(CNC(=O)C2)C1]

### Step 1: 2-(4-(4-bromo-1H-indol-1-yl)-2,6-dimethoxybenzyl)-2,6-diazaspiro[3.4]octan-7-one

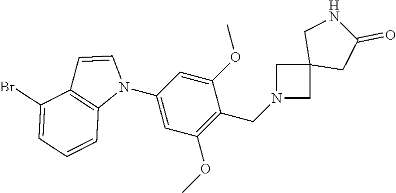 [SMILES: COc1cc(-n2ccc3c(Br)cccc32)cc(OC)c1CN1CC2(CNC(=O)C2)C1]

A solution of tert-butyl 7-oxo-2,6-diazaspiro[3.4]octane-2-carboxylate in dioxane was added with 4M HCl and stirred at room temperature for 1 hour. The solution was concentrated under reduced pressure to afford 2,6-diazaspiro[3.4]octan-7-one hydrochloride.

A mixture of Intermediate B1 (150.00 mg, 0.42 mmol) and 2,6-diazaspiro[3.4]octan-7-one hydrochloride (135.05 mg, 0.83 mmol) in 24 mL MeOH/DCM (1:1) was stirred at room temperature and added with acetic acid until the pH was around 4. After the reaction was stirred for 1 hour, sodium cyanoborohydride (131.96 mg, 2.10 mmol) was added and the reaction was stirred at room temperature for overnight. The reaction mixture was purified by column chromatography (silica gel, gradient elution, 0 to 5% MeOH/DCM). The fractions containing the product were collected and the solvent was removed under reduced pressure to provide 2-(4-(4-bromo-1H-indol-1-yl)-2,6-dimethoxybenzyl)-2,6-diazaspiro[3.4]octan-7-one.

### Step 2: 6-(2-chloro-3-(1-(3,5-dimethoxy-4-((7-oxo-2,6-diazaspiro[3.4]octan-2-yl)methyl)phenyl)-1H-indol-4-yl)phenyl)-2-methoxynicotinaldehyde

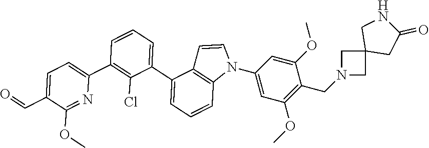 [SMILES: COc1cc(-n2ccc3c(-c4cccc(-c5ccc(C=O)c(OC)n5)c4Cl)cccc32)cc(OC)c1CN1CC2(CNC(=O)C2)C1]

A mixture of 2-(4-(4-bromo-1H-indol-1-yl)-2,6-dimethoxybenzyl)-2,6-diazaspiro[3.4]octan-7-one (100.00 mg, 0.21 mmol), Intermediate C2 (95.33 mg, 0.26 mmol), Pd(PPh₃)₄ (24.27 mg, 0.02 mmol) and K₂CO₃ (87.07 mg, 0.63 mmol) in a mixture of 1,4-dioxane and H₂O (6:1) 20 mL was sparged with argon for 10 min before stirring at 95° C. for 1.5 h under argon atmosphere. Then, the mixture was cooled down and diluted with brine. The resulting mixture was extracted 3 times with EtOAc and the organic layer was collected, dried over anhydrous Na₂SO₄, filtered, and evaporated to dryness. The crude mixture was purified by column chromatography (silica gel, gradient elution, 0 to 10% MeOH/DCM) to provide 6-(2-chloro-3-(1-(3,5-dimethoxy-4-((7-oxo-2,6-diazaspiro[3.4]octan-2-yl)methyl)phenyl)-1H-indol-4-yl)phenyl)-2-methoxynicotinaldehyde.

### Step 3: 2-(4-(4-(2-chloro-3-(5-((3-hydroxy-3-methylazetidin-1-yl)methyl)-6-methoxypyridin-2-yl)phenyl)-1H-indol-1-yl)-2,6-dimethoxybenzyl)-2,6-diazaspiro[3.4]octan-7-one

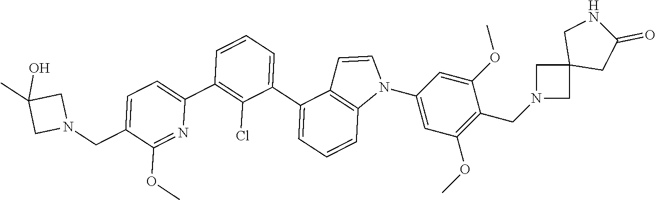 [SMILES: COc1cc(-n2ccc3c(-c4cccc(-c5ccc(CN6CC(C)(O)C6)c(OC)n5)c4Cl)cccc32)cc(OC)c1CN1CC2(CNC(=O)C2)C1]

A mixture of 6-(2-chloro-3-(1-(3,5-dimethoxy-4-((7-oxo-2,6-diazaspiro[3.4]octan-2-yl)methyl)phenyl)-1H-indol-4-yl)phenyl)-2-methoxynicotinaldehyde (35.00 mg, 0.05 mmol) and 3-methylazetidin-3-ol hydrochloride (8.11 mg, 0.07 mmol) in 6 mL MeOH/DCM (1:1) was stirred at room temperature and added with acetic acid until the pH was around 4. After the reaction was stirred for 1 hour, sodium cyanoborohydride (15.71 mg, 0.25 mmol) was added and the reaction was stirred at room temperature for 1 hour. The crude mixture was purified by Sephadex column chromatography (100% MeOH). The fractions containing the product were collected and the solvent was removed under reduced pressure to provide the title compound. LCMS Method B, RT=3.077 min, m/z=708.3 [M+H]⁺, LCMS Method D, RT=3.666 min, m/z=708.2892 [M+H]⁺, exact mass: 707.2874.

### Example 85: (S)-5-(((2,6-dimethoxy-4-(4-(2-methyl-3-(4,5,6,7-tetrahydrothiazolo[5,4-c]pyridin-2-yl)phenyl)indolin-1-yl)benzyl)amino)methyl)pyrrolidin-2-one

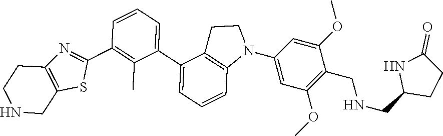 [SMILES: COc1cc(N2CCc3c(-c4cccc(-c5nc6c(s5)CNCC6)c4C)cccc32)cc(OC)c1CNC[C@@H]1CCC(=O)N1]

### Step 1: 2,2′-(2-methyl-1,3-phenylene)bis(4,4,5,5-tetramethyl-1,3,2-dioxaborolane)

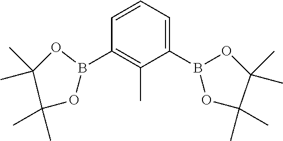

Pd(dppf)Cl₂·CH₂Cl₂ (0.20 g, 0.25 mmol) was added to a solution of 1,3-dibromo-2-methylbenzene (1.26 g, 5.05 mmol), B₂Pin₂ (3.08 g, 12.12 mmol), and KOAc (3.42 g, 34.8 mmol) in DMF 15 mL. The resulting suspension was sparged with argon for 10 min. Then, the reaction was sealed and stirred at 150° C. for 1 hour under argon atmosphere. After that, the mixture was cooled down and diluted with water. The resulting mixture was extracted 3 times with EtOAc and the organic layer was collected, dried over anhydrous Na₂SO₄, filtered, and evaporated to dryness. The product was further used without purification.

### Step 2: 4-(2-methyl-3-(4,4,5,5-tetramethyl-1,3,2-dioxaborolan-2-yl)phenyl)indoline

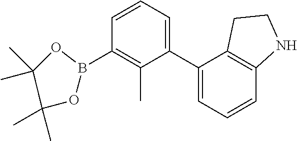 [SMILES: Cc1c(B2OC(C)(C)C(C)(C)O2)cccc1-c1cccc2c1CCN2]

A mixture of 2,2′-(2-methyl-1,3-phenylene)bis(4,4,5,5-tetramethyl-1,3,2-dioxaborolane) (1.74 g, 5.05 mmol), 4-bromoindoline (0.83 g, 4.21 mmol), Pd(PPh₃)₄ (0.58 g, 0.51 mmol) and K₂CO₃ (1.16 g, 8.42 mmol) in a mixture of 1,4-dioxane and H₂O (6:1) 35 mL was sparged with argon for 10 min before it was stirred at 95° C. for 3 h under argon atmosphere. Then, the mixture was cooled down and diluted with brine. The resulting mixture was extracted 3 times with EtOAc and the organic layer was collected, dried over anhydrous Na₂SO₄, filtered, and evaporated to dryness. The crude mixture was purified by column chromatography (silica gel, gradient elution, 20 to 80% EtOAc/hexanes) to provide the product.

### Step 3: tert-butyl 2-(3-(indolin-4-yl)-2-methylphenyl)-3a,6,7,7a-tetrahydrothiazolo[5,4-c]pyridine-5 (4H)-carboxylate

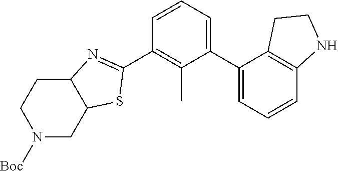 [SMILES: Cc1c(C2=NC3CCN(C(=O)OC(C)(C)C)CC3S2)cccc1-c1cccc2c1CCN2]

A mixture of 4-(2-methyl-3-(4,4,5,5-tetramethyl-1,3,2-dioxaborolan-2-yl)phenyl)indoline (0.24 g, 0.72 mmol), tert-butyl 2-bromo-3a,6,7,7a-tetrahydrothiazolo[5,4-c]pyridine-5 (4H)-carboxylate (0.20 g, 0.60 mmol), Pd(PPh₃)₄ (0.07 g, 0.06 mmol) and K₂CO₃ (0.17 g, 1.2 mmol) in a mixture of 1,4-dioxane and H₂O (6:1) 24 mL was sparged with argon for 10 min before it was stirred at 95° C. for 3 h under argon atmosphere. Then, the mixture was cooled down and diluted with brine. The resulting mixture was extracted 3 times with EtOAc and the organic layer was collected, dried over anhydrous Na₂SO₄, filtered, and evaporated to dryness. The crude mixture was purified by column chromatography (silica gel, gradient elution, 20 to 80% EtOAc/hexanes) to provide the product.

### Step 4: tert-butyl 2-(3-(1-(4-formyl-3,5-dimethoxyphenyl)indolin-4-yl)-2-methylphenyl)-3a,6,7,7a-tetrahydrothiazolo[5,4-c]pyridine-5 (4H)-carboxylate

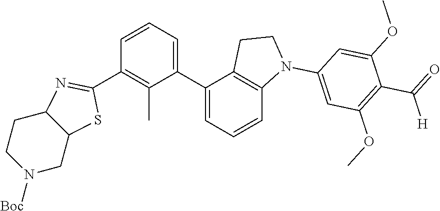 [SMILES: COc1cc(N2CCc3c(-c4cccc(C5=NC6CCN(C(=O)OC(C)(C)C)CC6S5)c4C)cccc32)cc(OC)c1C=O]

A mixture of tert-butyl 2-(3-(indolin-4-yl)-2-methylphenyl)-3a,6,7,7a-tetrahydrothiazolo[5,4-c]pyridine-5 (41)-carboxylate (0.22 g, 0.50 mmol), 4-bromo-2,6-dimethoxybenzaldehyde (0.15 g, 0.60 mmol), Pd(dba) (0.02 g, 0.03 mmol), Xantphos (0.03 g, 0.05 mmol) and Cs₂CO₃ (0.57 g, 1.75 mmol) in toluene 15 mL was sparged with argon for 10 min before it was refluxed under argon atmosphere for 16 h. The resulting mixture was cooled down, filtered, and evaporated to dryness. The crude mixture was purified by column chromatography (silica gel, gradient elution, 20 to 80% EtOAc/hexanes).

### Step 5: (S)-5-(((2,6-dimethoxy-4-(4-(2-methyl-3-(4,5,6,7-tetrahydrothiazolo[5,4-c]pyridin-2-yl)phenyl)indolin-1-yl)benzyl)amino)methyl)pyrrolidin-2-one

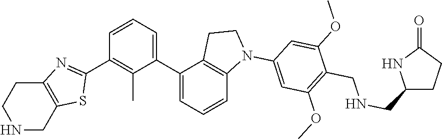 [SMILES: COc1cc(N2CCc3c(-c4cccc(-c5nc6c(s5)CNCC6)c4C)cccc32)cc(OC)c1CNC[C@@H]1CCC(=O)N1]

A mixture of tert-butyl 2-(3-(1-(4-formyl-3,5-dimethoxyphenyl)indolin-4-yl)-2-methylphenyl)-3a,6,7,7a-tetrahydrothiazolo[5,4-c]pyridine-5 (4H)-carboxylate (0.12 g, 0.20 mmol) and (S)-5-(aminomethyl)pyrrolidin-2-one (0.07 g, 0.60 mmol) in 4 mL MeOH/DCM (1:1) was stirred at room temperature and added with acetic acid until the pH was around 4. After the reaction was stirred for 1 hour, sodium cyanoborohydride (0.06 g, 1.00 mmol) was added and the reaction was stirred at room temperature for an additional 1 hour. The crude mixture was purified by column chromatography (silica gel, gradient elution, 10 to 15% MeOH/DCM). The fractions containing the product were collected and the solvent was removed under reduced pressure. The resulting oil was treated with 4M HCl in dioxane and stirred at room temperature for 30 min.

After evaporation to dryness, the crude mixture was purified by column chromatography (100% MeOH). The fractions containing the product were collected and the solvent was removed under reduced pressure, resulting the title compound. LCMS Method C, RT=3.8920 min, m/z=632.3 [M+Na]⁺, LCMS Method D, RT=3.490 min, m/z=610.2829 [M+H]⁺, exact mass: 609.2800.

### Example 86: 1-((2-(4-(2,3-dihydrobenzo[b][1,4]dioxin-6-yl)-1H-indol-1-yl)-6-methylpyridin-4-yl)methyl)-3-methylazetidine-3-carboxylic acid

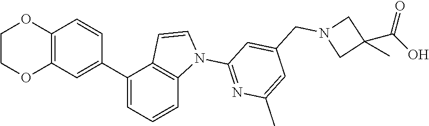 [SMILES: Cc1cc(CN2CC(C)(C(=O)O)C2)cc(-n2ccc3c(-c4ccc5c(c4)OCCO5)cccc32)n1]

### Step 1: 2-(4-(2,3-dihydrobenzo[b][1,4]dioxin-6-yl)-1H-indol-1-yl)-6-methylisonicotinaldehyde

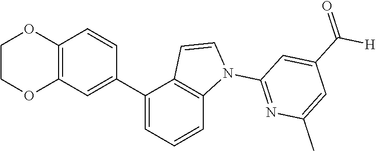 [SMILES: Cc1cc(C=O)cc(-n2ccc3c(-c4ccc5c(c4)OCCO5)cccc32)n1]

A mixture of Intermediate A1 (0.06 g, 0.23 mmol), 2-bromo-6-methylisonicotinaldehyde (0.06 g, 0.30 mmol), Pd(dba); (0.01 g, 0.01 mmol), Xantphos (0.01 g, 0.02 mmol) and Cs₂CO₃ (0.26 g, 0.81 mmol) in toluene 15 mL was sparged with argon for 10 min before it was refluxed under argon atmosphere for 16 h. The resulting mixture was cooled down, filtered, and evaporated to dryness. The crude mixture was purified by column chromatography (silica gel, gradient elution, 20 to 80% EtOAc/hexanes).

### Step 2: 1-((2-(4-(2,3-dihydrobenzo[b][1,4]dioxin-6-yl)-1H-indol-1-yl)-6-methylpyridin-4-yl)methyl)-3-methylazetidine-3-carboxylic acid

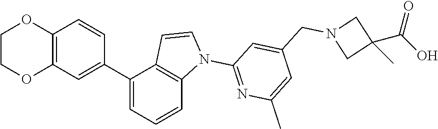 [SMILES: Cc1cc(CN2CC(C)(C(=O)O)C2)cc(-n2ccc3c(-c4ccc5c(c4)OCCO5)cccc32)n1]

A mixture of 2-(4-(2,3-dihydrobenzo[b][1,4]dioxin-6-yl)-1H-indol-1-yl)-6-methylisonicotinaldehyde (0.06 g, 0.17 mmol) and 3-methylazetidine-3-carboxylic acid (0.02 g, 0.20 mmol) in 4 mL MeOH/DCM (1:1) was stirred at room temperature and added with acetic acid until the pH was around 4. After the reaction was stirred for 1 hour, sodium cyanoborohydride (0.03 g, 0.51 mmol) was added and the reaction was stirred at room temperature for an additional 1 hour. After evaporation to dryness, the resulting crude mixture was stirred at room temperature in the presence of 4M HCl in dioxane for 30 min. All solvent was evaporated under vacuum and the crude mixture was purified by column chromatography (silica gel, gradient elution, 5 to 25% MeOH/DCM). The fractions containing the product were collected and the solvent was removed under reduced pressure, resulting in the title compound. LCMS Method A, RT=2.837 min, m/z=470.2 [M+H]⁺, LCMS Method D, RT=4.196 min, m/z=470.2043 [M+H]⁺, exact mass: 469.2001.

### Example 87: (S)-5-(((4-(4-(2-chloro-3-(5-(((R)-3-hydroxypyrrolidin-1-yl)methyl)-6-methoxypyridin-2-yl)phenyl)-1H-indazol-1-yl)-2,6-dimethoxybenzyl)amino)methyl) pyrrolidin-2-one

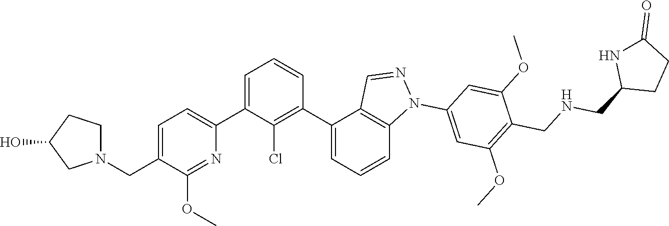 [SMILES: COc1cc(-n2ncc3c(-c4cccc(-c5ccc(CN6CC[C@@H](O)C6)c(OC)n5)c4Cl)cccc32)cc(OC)c1CNC[C@@H]1CCC(=O)N1]

A mixture of Intermediate F2 (0.16 g, 0.20 mmol) and (S)-5-(aminomethyl)pyrrolidin-2-one (0.03 g, 0.24 mmol) in 4 mL MeOH/DCM (1:1) was stirred at room temperature and added with acetic acid until the pH was around 4. After the reaction was stirred for 1 hour, sodium cyanoborohydride (0.06 g, 1.00 mmol) was added and the reaction was stirred at room temperature for an additional 1 hour. The crude mixture was purified by column chromatography (silica gel, gradient elution, 10 to 50% MeOH/DCM). The fractions containing the product were collected and the solvent was removed under reduced pressure. The resulting oil was treated with 4M HCl in dioxane and stirred at room temperature for 30 min. After evaporation to dryness, the crude mixture was purified by Sephadex column chromatography (100% MeOH). The fractions containing the product were collected and the solvent was removed under reduced pressure, resulting in the title compound. LCMS Method B, RT=3.083 min, m/z=697.3 [M+H]⁺, LCMS Method D, RT=3.581 min, m/z=697.2908 [M+H]⁺, exact mass: 696.2800.

### Example 88: (S)-1-(4-(4-(2-chloro-3-(6-methoxy-5-((((5-oxopyrrolidin-2-yl)methyl)amino) methyl)pyridin-2-yl)phenyl)-1H-indazol-1-yl)-2,6-dimethoxybenzyl)-N-cyclopropyl-3-methylazetidine-3-carboxamide

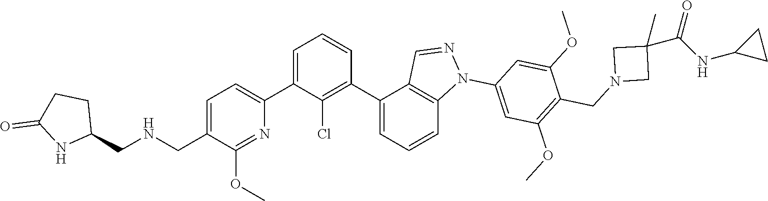 [SMILES: COc1cc(-n2ncc3c(-c4cccc(-c5ccc(CNC[C@@H]6CCC(=O)N6)c(OC)n5)c4Cl)cccc32)cc(OC)c1CN1CC(C)(C(=O)NC2CC2)C1]

### Step 1: 1-(4-(4-bromo-1H-indazol-1-yl)-2,6-dimethoxybenzyl)-N-cyclopropyl-3-methylazetidine-3-carboxamide

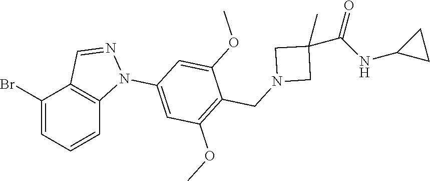 [SMILES: COc1cc(-n2ncc3c(Br)cccc32)cc(OC)c1CN1CC(C)(C(=O)NC2CC2)C1]

N-cyclopropyl-3-methylazetidine-3-carboxamide was prepared as described in Step 1 of Intermediate C1-9. A mixture of Intermediate B4 (0.20 g, 0.55 mmol) and N-cyclopropyl-3-methylazetidine-3-carboxamide (0.17 g, 1.10 mmol) in 4 mL MeOH/DCM (1:1) was stirred at room temperature and added with acetic acid until the pH was around 4. After the reaction was stirred for 1 hour, sodium cyanoborohydride (0.10 g, 1.65 mmol) was added and the reaction was stirred at room temperature for an additional 1 hour. After evaporation to dryness, the resulting crude mixture was stirred at room temperature in the presence of 4M HCl in dioxane for 30 min. All solvent was evaporated under vacuum and the crude mixture was purified by column chromatography (silica gel, gradient elution, 10 to 100% MeOH/DCM).

### Step 2: 1-(4-(4-(2-chloro-3-(5-formyl-6-methoxypyridin-2-yl)phenyl)-1H-indazol-1-yl)-2,6-dimethoxybenzyl)-N-cyclopropyl-3-methylazetidine-3-carboxamide

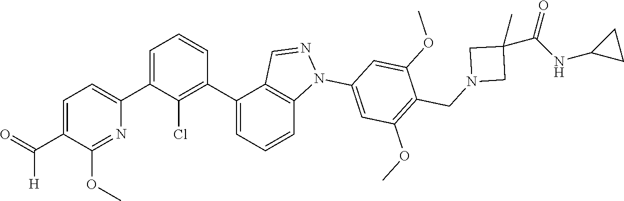 [SMILES: COc1cc(-n2ncc3c(-c4cccc(-c5ccc(C=O)c(OC)n5)c4Cl)cccc32)cc(OC)c1CN1CC(C)(C(=O)NC2CC2)C1]

A mixture of Intermediate C2 (0.26 g, 0.70 mmol), 1-(4-(4-bromo-1H-indazol-1-yl)-2,6-dimethoxybenzyl)-N-cyclopropyl-3-methylazetidine-3-carboxamide (0.40 g, 0.80 mmol), Pd(PPh₃)₄ (0.16 g, 0.14 mmol) and K₂CO₃ (2.32 g, 16.8 mmol) in a mixture of 1,4-dioxane and H₂O (6:1) 14 mL was sparged with argon for 10 min before it was stirred at 95° C. for 3 h under argon atmosphere. Then, the mixture was cooled down and diluted with brine. The resulting mixture was extracted 3 times with EtOAc and the organic layer was collected, dried over anhydrous Na₂SO₄, filtered, and evaporated to dryness. The crude mixture was purified by column chromatography (silica gel, gradient elution, 20 to 100% MeOH/DCM).

### Step 3: (S)-1-(4-(4-(2-chloro-3-(6-methoxy-5-((((5-oxopyrrolidin-2-yl)methyl)amino) methyl)pyridin-2-yl)phenyl)-1H-indazol-1-yl)-2,6-dimethoxybenzyl)-N-cyclopropyl-3-methylazetidine-3-carboxamide

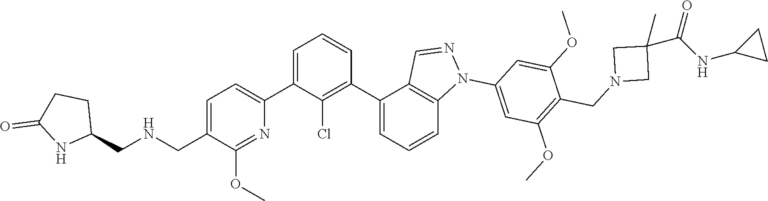 [SMILES: COc1cc(-n2ncc3c(-c4cccc(-c5ccc(CNC[C@@H]6CCC(=O)N6)c(OC)n5)c4Cl)cccc32)cc(OC)c1CN1CC(C)(C(=O)NC2CC2)C1]

A mixture of 1-(4-(4-(2-chloro-3-(5-formyl-6-methoxypyridin-2-yl)phenyl)-1H-indazol-1-yl)-2,6-dimethoxybenzyl)-N-cyclopropyl-3-methylazetidine-3-carboxamide (0.11 g, 0.17 mmol) and (S)-5-(aminomethyl)pyrrolidin-2-one (0.02 g, 0.20 mmol) in 4 mL MeOH/DCM (1:1) was stirred at room temperature and added with acetic acid until the pH was around 4. After the reaction was stirred for 1 hour, sodium cyanoborohydride (0.06 g, 1.00 mmol) was added and the reaction was stirred at room temperature for an additional 1 hour. The crude mixture was purified by column chromatography (silica gel, 100/MeOH/DCM). The fractions containing the product were collected and the solvent was removed under reduced pressure, resulting the title compound. LCMS Method B, RT=3.090 min, m/z=764.4 [M+H]⁺, LCMS Method D, RT=3.669 min, m/z=764.3319 [M+H]⁺, exact mass: 763.3200.

### Example 89: 1-(4-(4-(2-chloro-3-(5-(((((cis)-3-hydroxycyclobutyl)methyl)amino)methyl)-6-methoxypyridin-2-yl)phenyl)-1H-indazol-1-yl)-2,6-dimethoxybenzyl)-N-cyclopropyl-3-methylazetidine-3-carboxamide

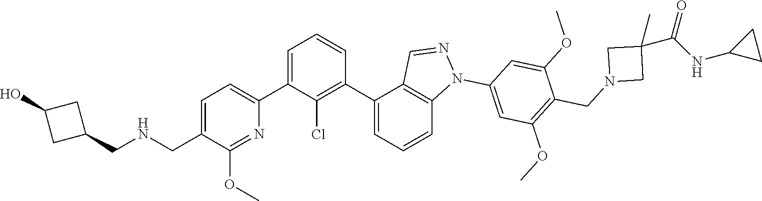 [SMILES: COc1cc(-n2ncc3c(-c4cccc(-c5ccc(CNC[C@H]6C[C@@H](O)C6)c(OC)n5)c4Cl)cccc32)cc(OC)c1CN1CC(C)(C(=O)NC2CC2)C1]

An intermediate 1-(4-(4-(2-chloro-3-(5-formyl-6-methoxypyridin-2-yl)phenyl)-1H-indazol-1-yl)-2,6-dimethoxybenzyl)-N-cyclopropyl-3-methylazetidine-3-carboxamide was prepared by following the procedures as described in Example 88. A mixture of 1-(4-(4-(2-chloro-3-(5-formyl-6-methoxypyridin-2-yl)phenyl)-1H-indazol-1-yl)-2,6-dimethoxybenzyl)-N-cyclopropyl-3-methylazetidine-3-carboxamide (0.11 g, 0.17 mmol) and cis-3-(aminomethyl)cyclobutan-1-ol (0.02 g, 0.20 mmol) in 4 mL MeOH/DCM (1:1) was stirred at room temperature and added with acetic acid until the pH was around 4. After the reaction was stirred for 1 hour, sodium cyanoborohydride (0.06 g, 1.00 mmol) was added and the reaction was stirred at room temperature for an additional 1 hour. The crude mixture was purified by column chromatography (silica gel, gradient elution, 10 to 100% MeOH/DCM). The fractions containing the product were collected and the solvent was removed under reduced pressure, resulting the title compound. LCMS Method B, RT=3.136 min, m/z=751.4 [M+H]⁺, LCMS Method D, RT=3.744 min, m/z=751.3361 [M+H]⁺, exact mass: 750.3300.

### Example 90: 2-(4-(4-(2-chloro-3-(6-methoxy-5-((7-oxo-2,6-diazaspiro[3.4]octan-2-yl)methyl)pyridin-2-yl)phenyl)-1H-indazol-1-yl)-2,6-dimethoxybenzyl)-2,6-diazaspiro[3.4]octan-7-one

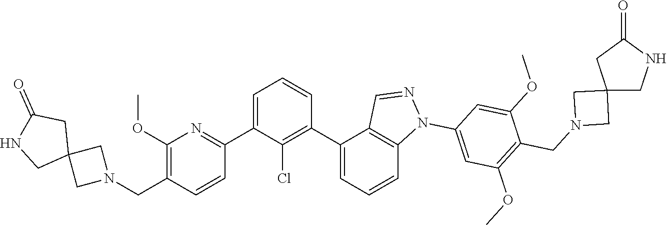 [SMILES: COc1cc(-n2ncc3c(-c4cccc(-c5ccc(CN6CC7(CNC(=O)C7)C6)c(OC)n5)c4Cl)cccc32)cc(OC)c1CN1CC2(CNC(=O)C2)C1]

NEt₃ was added to a mixture of 2,6-diazaspiro[3.4]octan-7-one bis(4-methylbenzenesulfonate) (0.06 g, 0.13 mmol) in 3 mL MeOH/DCM (1:1) until the pH was around 7. Then, the mixture was transferred to a mixture of Intermediate D2 (0.03 g, 0.06 mmol) in 1 mL MeOH/DCM (1:1) at room temperature. After that, acetic acid was added until the pH was around 4. After the reaction was stirred for 1 hour, sodium cyanoborohydride (0.03 g, 0.44 mmol) was added and the reaction was stirred at room temperature for 2 hours. The crude mixture was purified by Sephadex column chromatography (100% MeOH). The fractions containing the product were collected and the solvent was removed under reduced pressure, resulting in the title compound. LCMS Method B, RT=3.014 min, m/z=748.4 [M+H]⁺, LCMS Method D, RT=3.472 min, m/z=748.3015 [M+H]⁺, exact mass: 747.2936.

### Example 91: 2-((6-(2-chloro-3-(1-(4-(((((cis)-3-hydroxycyclobutyl)methyl)amino)methyl)-3,5-dimethoxyphenyl)-1H-indazol-4-yl)phenyl)-2-methoxypyridin-3-yl)methyl)-2,6-diazaspiro[3.4]octan-7-one

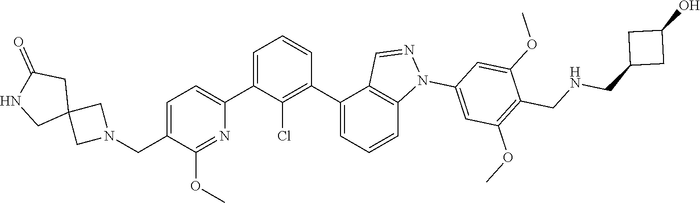 [SMILES: COc1cc(-n2ncc3c(-c4cccc(-c5ccc(CN6CC7(CNC(=O)C7)C6)c(OC)n5)c4Cl)cccc32)cc(OC)c1CNC[C@H]1C[C@@H](O)C1]

A mixture of Intermediate E6 (0.11 g, 0.17 mmol) and cis-3-(aminomethyl)cyclobutan-1-ol (0.02 g, 0.20 mmol) in 4 mL MeOH/DCM (1:1) was stirred at room temperature added with acetic acid until the pH was around 4. After the reaction was stirred for 1 hour, sodium cyanoborohydride (0.06 g, 1.00 mmol) was added and the reaction was stirred at room temperature for an additional 1 hour. The crude mixture was purified by column chromatography (silica gel, gradient elution, 5 to 100% MeOH/DCM). The fractions containing the product were collected and the solvent was removed under reduced pressure, resulting in the title compound.

LCMS Method B, RT=3.037 min, m/z=723.4 [M+H]⁺, LCMS Method D, RT=3.566 min, m/z=723.3040 [M+H]⁺, exact mass: 722.2983.

Example 92: (S)-1-((6-(2-chloro-3-(1-(3,5-dimethoxy-4-((((5-oxopyrrolidin-2-yl)methyl)amino)methyl)phenyl)-1H-indazol-4-yl)phenyl)-2-methoxypyridin-3-yl)methyl)-N-cyclopropyl-3-methylazetidine-3-carboxamide

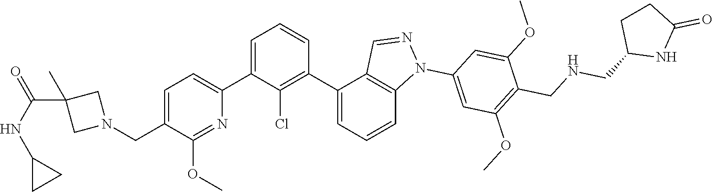 [SMILES: COc1cc(-n2ncc3c(-c4cccc(-c5ccc(CN6CC(C)(C(=O)NC7CC7)C6)c(OC)n5)c4Cl)cccc32)cc(OC)c1CNC[C@@H]1CCC(=O)N1]

N-cyclopropyl-3-methylazetidine-3-carboxamide was prepared as described in Step 1 of Intermediate C1-9. A mixture of Intermediate F2 (0.12 g, 0.17 mmol) and N-cyclopropyl-3-methylazetidine-3-carboxamide (0.03 g, 0.20 mmol) in 4 mL MeOH/DCM (1:1) was stirred at room temperature and acetic acid was added until the pH was around 4. After the reaction was stirred for 1 hour, sodium cyanoborohydride (0.03 g, 0.51 mmol) was added and the reaction was stirred at room temperature for an additional 1 hour. After evaporation to dryness, the resulting crude mixture was stirred at room temperature in the presence of 4M HCl in dioxane for 30 min. All solvent was evaporated under vacuum and the crude mixture was purified by column chromatography (silica gel, gradient elution, 5 to 25% MeOH/DCM). The fractions containing the product were collected and the solvent was removed under reduced pressure, resulting in the title compound. LCMS Method B, RT=3.077 min, m/z=764.4 [M+H]⁺, LCMS Method D, RT=3.593 min, m/z=[M+H]⁺, LCMS Method D, RT=3.645 min, m/z=764.3310 [M+H]⁺, exact mass: 763.3249.

Example 93: (S)-5-((((6-(2-chloro-3-(3-(3,5-dimethoxy-4-(((((S)-5-oxopyrrolidin-2-yl)methyl)amino)methyl)phenyl)-1-methyl-1H-indol-7-yl)phenyl)-2-methoxypyridin-3-yl)methyl)amino)methyl)pyrrolidin-2-one

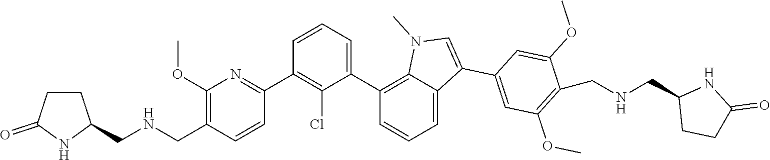 [SMILES: COc1cc(-c2cn(C)c3c(-c4cccc(-c5ccc(CNC[C@@H]6CCC(=O)N6)c(OC)n5)c4Cl)cccc23)cc(OC)c1CNC[C@@H]1CCC(=O)N1]

### Step 1: 7-bromo-1-methyl-1H-indole

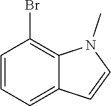

NaH (0.16 g, 6.5 mmol) was added into a solution of 7-bromo-1H-indole (0.50 g, 2.42 mmol) in DMF (4 mL). After stirring at room temperature for 1 hour, the solution was cooled down in an ice bath. Methyl iodide (0.20 mL, 3.2 mmol) was added into the solution and the reaction was stirred for 30 min. The reaction was quenched by water and extracted with EtOAc. The organic layer was collected, dried over anhydrous Na₂SO₄, filtered, and evaporated to dryness. The product was further used without purification. The crude mixture was purified by column chromatography (silica gel, gradient elution, 10 to 20% EtOAc/hexanes) to provide the product.

### Step 2: 7-bromo-3-iodo-1-methyl-1H-indole

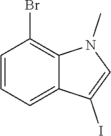

N-iodosuccinimide (1.12 g, 4.98 mmol) was added to a solution of 7-bromo-1-methyl-1H-indole (350 mg, 1.66 mmol) in DMF (5 mL). The resulting suspension was sparged with argon for 10 min. After the reaction was sealed and stirred at 100° C. for 16 h under argon atmosphere, it was cooled down and quenched with sodium thiosulfate. The resulting mixture was extracted 3 times with EtOAc and the organic layer was collected, dried over anhydrous Na₂SO₄, filtered, and evaporated to dryness to provide the product. The product was further used without purification.

### Step 3: (S)-5-((((6-(2-chloro-3-(3-(3,5-dimethoxy-4-(((((S)-5-oxopyrrolidin-2-yl)methyl)amino)methyl)phenyl)-1-methyl-1H-indol-7-yl)phenyl)-2-methoxypyridin-3-yl)methyl)amino)methyl)pyrrolidin-2-one

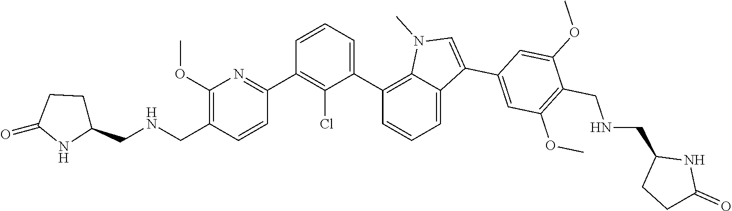 [SMILES: COc1cc(-c2cn(C)c3c(-c4cccc(-c5ccc(CNC[C@@H]6CCC(=O)N6)c(OC)n5)c4Cl)cccc23)cc(OC)c1CNC[C@@H]1CCC(=O)N1]

This step was prepared by using similar procedures as described in the Example 31. An intermediate 7-bromo-3-iodo-1-methyl-1H-indazole was replaced with 7-bromo-3-iodo-1-methyl-1H-indole to obtain the desired product. LCMS Method B, RT=3.087 min, m/z=737.3 [M+H]⁺, LCMS Method D, RT=3.655 min, m/z=737.3222 [M+H]⁺, exact mass: 736.3100.

### Example 94: (S)-2-((6-(2-chloro-3-(1-(3,5-dimethoxy-4-((((5-oxopyrrolidin-2-yl)methyl)amino)methyl)phenyl)-1H-indazol-4-yl)phenyl)-2-methoxypyridin-3-yl)methyl)-2,6-diazaspiro[3.4]octan-7-one

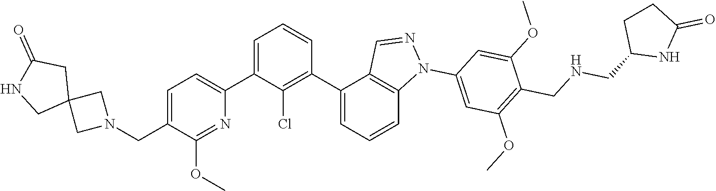 [SMILES: COc1cc(-n2ncc3c(-c4cccc(-c5ccc(CN6CC7(CNC(=O)C7)C6)c(OC)n5)c4Cl)cccc32)cc(OC)c1CNC[C@@H]1CCC(=O)N1]

A mixture of Intermediate E6 (30.0 mg, 0.047 mmol) and (S)-5-(aminomethyl)pyrrolidin-2-one (5.6 mg, 0.049 mmol) in 4 mL MeOH/DCM (1:1) was stirred at room temperature and added with acetic acid until the pH was around 4. After the reaction was stirred for 1.5 hour, sodium cyanoborohydride (15.7 mg, 0.25 mmol) was added and the reaction was stirred at room temperature for an additional 30 min. The crude mixture was purified by column chromatography (silica gel, gradient elution, 5 to 30% MeOH/DCM with addition of 0.5% TEA). The fractions containing the product were collected and the solvent was removed under reduced pressure, resulting the title compound. LCMS Method B, RT=3.013 min, m/z=736.3 [M+H]⁺, 758.2 [M+Na]⁺, LCMS Method D, RT=3.456 min, m/z=736.3032 [M+H]⁺, exact mass: 735.2936. ¹H NMR (500 MHz, MeOD-d4) S ppm 8.02 (s, 1H), 7.93 (d, J=10.0 Hz, 1H), 7.83 (d, J=7.5 Hz, 1H), 7.67 (dd, J=7.5, 2.0 Hz, 1H), 7.62 (dd, J=7.5, 2.0 Hz, 1H), 7.53 (m, 3H) 7.28 (d, J=7.5 Hz, 1H), 7.12 (s, 2H), 4.17 (s, 1H), 4.04 (s, 3H), 3.98 (s, 6H), 3.95 (s, 1H), 3.66 (s, 2H), 3.32 (m, 5H), 3.28 (m, 2H), 3.22-3.10 (m, 4H), 2.45-2.29 (m, 1H), 2.13 (s, 2H), 1.94-1.82 (m, 1H).

Example 95: 1-((6-(2-chloro-3-(1-(4-((((cis-3-hydroxycyclobutyl)methyl) amino)methyl)-3,5-dimethoxyphenyl)-1H-indazol-4-yl)phenyl)-2-methoxypyridin-3-yl)methyl)-N-cyclopropyl-3-methylazetidine-3-carboxamide

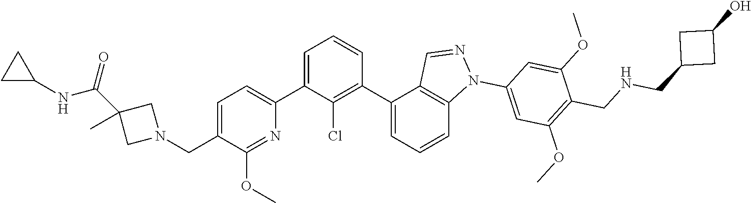 [SMILES: COc1cc(-n2ncc3c(-c4cccc(-c5ccc(CN6CC(C)(C(=O)NC7CC7)C6)c(OC)n5)c4Cl)cccc32)cc(OC)c1CNC[C@H]1C[C@@H](O)C1]

A mixture of Intermediate E8 (0.13 g, 0.17 mmol) and cis-3-(aminomethyl)cyclobutan-1-ol (0.02 g, 0.20 mmol) in 4 mL MeOH/DCM (1:1) was stirred at room temperature and acetic acid was added until the pH was around 4. After the reaction was stirred for 1 hour, sodium cyanoborohydride (0.03 g, 0.51 mmol) was added and the reaction was stirred at room temperature for an additional 1 hour. The crude mixture was purified by column chromatography (silica gel, gradient elution, 5 to 25% MeOH/DCM). The fractions containing the product were collected and the solvent was removed under reduced pressure, resulting in the title compound. LCMS Method B, RT=3.140 min, m/z=751.4 [M+H]⁺, LCMS Method D, RT=3.930 min, m/z=751.3366 [M+H]⁺, exact mass: 750.3296.

### Example 96: (S)-2-((6-(2-chloro-3-(1-(3,5-dimethoxy-4-((((5-oxopyrrolidin-2-yl)methyl)amino)methyl)phenyl)-1H-indol-4-yl)phenyl)-2-methoxypyridin-3-yl)methyl)-2,6-diazaspiro[3.4]octan-7-one

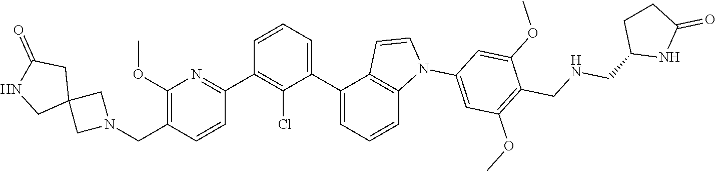 [SMILES: COc1cc(-n2ccc3c(-c4cccc(-c5ccc(CN6CC7(CNC(=O)C7)C6)c(OC)n5)c4Cl)cccc32)cc(OC)c1CNC[C@@H]1CCC(=O)N1]

A mixture of Intermediate E11 (80 mg, 0.13 mmol) and (S)-5-(aminomethyl)pyrrolidin-2-one (17 mg, 0.15 mmol) in 5 mL MeOH/DCM (1:1) was stirred at room temperature and added with acetic acid until the pH was around 4. After the reaction was stirred for 1 hour, sodium cyanoborohydride (40 mg, 0.63 mmol) was added and the reaction was stirred at room temperature for an additional 1 hour. The crude mixture was purified by column chromatography (silica gel, gradient elution, 10 to 50% MeOH/DCM). The fractions containing the product were collected and the solvent was removed under reduced pressure, resulting in the title compound. LCMS Method B, RT=3.037 min, m/z=735.3 [M+H]⁺, m/z=757.3 [M+Na]⁺, LCMS Method D, RT=3.838 min, m/z=735.3157 [M+H]⁺, exact mass: 734.2983.

### Example 97: 2-((6-(2-chloro-3-(l-(4-(((cis-3-hydroxycyclobutyl)amino)methyl)-3,5-dimethoxyphenyl)-1H-indazol-4-yl)phenyl)-2-methoxypyridin-3-yl)methyl)-2,6-diazaspiro[3.4]octan-7-one

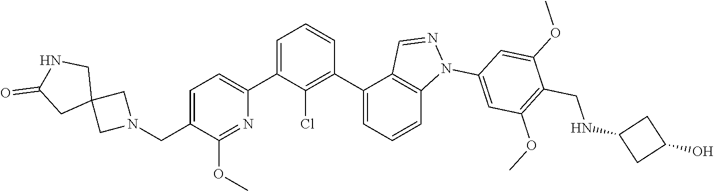 [SMILES: COc1cc(-n2ncc3c(-c4cccc(-c5ccc(CN6CC7(CNC(=O)C7)C6)c(OC)n5)c4Cl)cccc32)cc(OC)c1CN[C@H]1C[C@@H](O)C1]

A mixture of Intermediate E6 (45 mg, 0.07 mmol) and cis-3-aminocyclobutanol hydrochloride (9.88 mg, 0.08 mmol) in 6 mL MeOH/CH₂Cl₂ (1:1) was stirred at room temperature and acetic acid was added until pH value around 4. After leaving for 1 hour, sodium cyanoborohydride (21.99 mg, 0.35 mmol) was added and the reaction was stirred at room temperature for an additional 1 hour. The reaction mixture was purified by Sephadex column chromatography, affording the title compound. LCMS Method F, RT=6.162 min, m/z=709.3 [M+H]⁺, LCMS Method D, RT=3.556 min, m/z=709.2854 [M+H]⁺, exact mass: 708.2827.

### Example 98: (S)-5-(((4-(4-(2-chloro-3-(5-((((cis-3-hydroxycyclobutyl)amino) methyl)-6-methoxypyridin-2-yl)phenyl)-1H-indazol-1-yl)-2,6-dimethoxybenzyl)amino)methyl)pyrrolidin-2-one

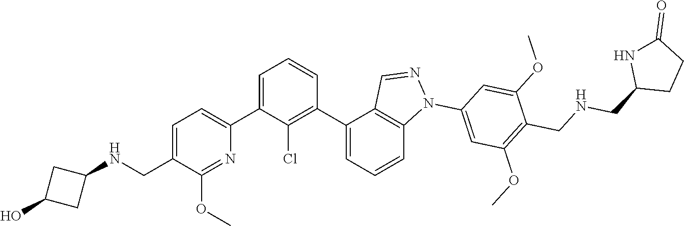 [SMILES: COc1cc(-n2ncc3c(-c4cccc(-c5ccc(CN[C@H]6C[C@@H](O)C6)c(OC)n5)c4Cl)cccc32)cc(OC)c1CNC[C@@H]1CCC(=O)N1]

A mixture of Intermediate E7 (0.12 g, 0.17 mmol) and (S)-5-(aminomethyl)pyrrolidin-2-one (0.02 g, 0.20 mmol) in 4 mL MeOH/DCM (1:1) was stirred at room temperature and acetic acid was added until the pH was around 4. After the reaction was stirred for 1 hour, sodium cyanoborohydride (0.03 g, 0.51 mmol) was added and the reaction was stirred at room temperature for an additional 1 hour. After evaporation to dryness, the resulting crude mixture was stirred at room temperature in the presence of 4M HCl in dioxane for 30 min. All solvent was evaporated under vacuum and the crude mixture was purified by column chromatography (silica gel, gradient elution, 5 to 25% MeOH/DCM). The fractions containing the product were collected and the solvent was removed under reduced pressure, resulting in the title compound. LCMS Method B, RT=3.038 min, m/z=697.4 [M+H]⁺, 719.3 [M+Na]⁺, LCMS Method D, RT=3.518 min, m/z=697.2895 [M+H]⁺, exact mass: 696.2827.

### Example 99: (S)-2-((6-(2-chloro-3-(1-(3,5-dimethoxy-4-((((5-oxopyrrolidin-2-yl)methyl)amino)methyl)phenyl)-1H-indazol-4-yl)phenyl)-2-methoxypyridin-3-yl)methyl)-2,5-diazaspiro[3.4]octan-6-one

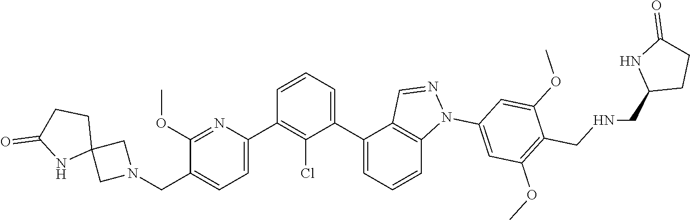 [SMILES: COc1cc(-n2ncc3c(-c4cccc(-c5ccc(CN6CC7(CCC(=O)N7)C6)c(OC)n5)c4Cl)cccc32)cc(OC)c1CNC[C@@H]1CCC(=O)N1]

A mixture of Intermediate E10 (50.0 mg, 0.08 mmol) and (S)-5-(aminomethyl)pyrrolidin-2-one (14.0 mg, 0.12 mmol) in 7 mL MeOH/DCM (1:1) was stirred at room temperature and added with acetic acid until the pH was around 4. After the reaction was stirred for 2 hours, sodium cyanoborohydride (37.0 mg, 0.59 mmol) was added and the reaction was stirred at room temperature for 18 hours. The crude mixture was purified by Sephadex column chromatography (100% MeOH). The fractions containing the product were collected and the solvent was removed under reduced pressure to provide the title compound. LCMS Method B, RT=3.016 min, m/z=736.4 [M+H]⁺, LCMS Method D, RT=3.719 min, m/z=736.3041 [M+H]⁺, exact mass: 735.2936. ¹H NMR (500 MHz, MeOD-d₄) δ ppm 8.00 (s, 1H), 7.88 (d, J=8.59 Hz, 1H), 7.68 (d, J=7.44 Hz, 2H), 7.64 (dd, J=2.02, 7.35 Hz, 1H), 7.59 (dd, J=7.19, 8.50 Hz, 1H), 7.52 (t, J=7.51 Hz, 1H), 7.49 (dd, J=2.04, 7.56 Hz, 1H), 7.25 (dd, J=3.18, 7.47 Hz, 1H), 7.05 (m, 1H), 7.04 (s, 2H), 4.60 (s, 1H), 3.99-3.84 (m, 2H), 3.98 (s, 3H), 3.94 (s, 6H), 3.70 (s, 2H), 3.54 (dd, J=1.66, 7.99 Hz, 2H), 3.35 (dd, J=1.70, 8.02 Hz, 2H), 2.43-2.24 (m, 5H), 1.37-1.17 (m, 2H), 0.89-0.82 (m, 3H).

### Example 100: 1-(4-(4-(2-chloro-3-(5-(((cis-3-hydroxycyclobutyl)amino)methyl)-6-methoxypyridin-2-yl)phenyl)-1H-indazol-1-yl)-2,6-dimethoxybenzyl)-N-cyclopropyl-3-methylazetidine-3-carboxamide

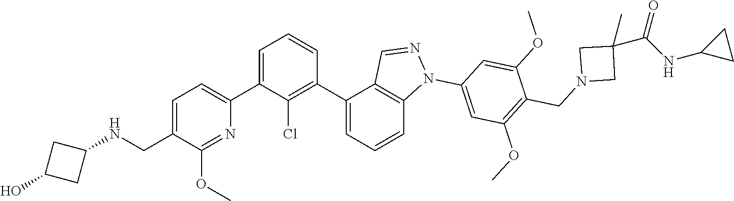 [SMILES: COc1cc(-n2ncc3c(-c4cccc(-c5ccc(CN[C@H]6C[C@@H](O)C6)c(OC)n5)c4Cl)cccc32)cc(OC)c1CN1CC(C)(C(=O)NC2CC2)C1]

N-cyclopropyl-3-methylazetidine-3-carboxamide was prepared as described in Step 1 of Intermediate C1-9. A mixture of Intermediate E7 (40.00 mg, 0.057 mmol) and N-cyclopropyl-3-methylazetidine-3-carboxamide (26.34 mg, 0.171 mmol) in 4 mL MeOH/DCM (1:1) was stirred at room temperature and added with acetic acid until the pH was around 4. After the reaction was stirred for 2 hours, sodium cyanoborohydride (18.27 mg, 0.29 mmol). The reaction was stirred at room temperature for an additional 1 hour, quenched by a small amount of MeOH and evaporated to dryness. Then, the residue was dissolved in DCM and trifluoroacetic acid (0.1 mL) was added. After stirring at room temperature for 30 minutes, the residual solvent was removed under reduced pressure. The crude mixture was purified by Sephadex column chromatography (100% MeOH), affording the title compound. LCMS method B, RT=3.103 min, m/z=737.3 [M+H]⁺, LCMS Method D, RT=3.929 min, m/z=737.3229 [M+H]⁺, exact mass: 736.3140.

### Example 101: cis-3-(((6-(2-chloro-3-(1-(4-(((cis-3-hydroxycyclobutyl)amino)methyl)-3,5-dimethoxyphenyl)-1H-indazol-4-yl)phenyl)-2-methoxypyridin-3-yl)methyl)amino)cylobutanol

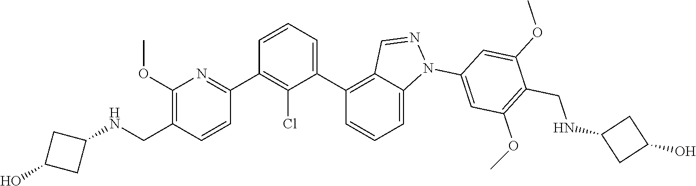 [SMILES: COc1cc(-n2ncc3c(-c4cccc(-c5ccc(CN[C@H]6C[C@@H](O)C6)c(OC)n5)c4Cl)cccc32)cc(OC)c1CN[C@H]1C[C@@H](O)C1]

A mixture of Intermediate D2 (29.4 mg, 0.06 mmol) and cis-3-aminocyclobutan-1-ol hydrochloride (8.8 mg, 0.10 mmol) in 2 mL MeOH/DCM (1:1) was stirred at room temperature and acetic acid was added until the pH was around 4. After the reaction was stirred for 1 hour, sodium cyanoborohydride (17.8 mg, 0.22 mmol) was added and the reaction was stirred at room temperature for an additional 1 hour. The crude mixture was purified by column chromatography (silica gel, gradient elution, 0 to 50% MeOH/DCM). The fractions containing the product were collected and the solvent was removed under reduced pressure, resulting in the title compound. LCMS Method B, RT=3.055 min, m/z=670.3 [M+H]⁺, LCMS Method D, RT=3.661 min, m/z=670.2788 [M+H]⁺, exact mass: 669.2718.

### Example 102: 1-(4-(4-(2-chloro-3-(5-(((cis-3-hydroxycyclobutyl)amino)methyl)-6-methoxypyridin-2-yl)phenyl)-1H-indazol-1-yl)-2,6-dimethoxybenzyl)-3-methylazetidin-3-ol

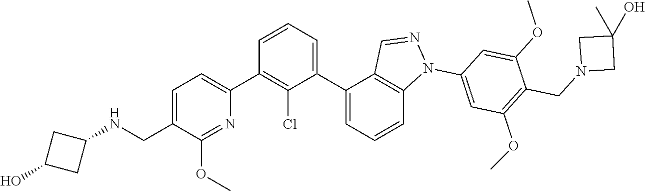 [SMILES: COc1cc(-n2ncc3c(-c4cccc(-c5ccc(CN[C@H]6C[C@@H](O)C6)c(OC)n5)c4Cl)cccc32)cc(OC)c1CN1CC(C)(O)C1]

A mixture of Intermediate E7 (83 mg, 0.12 mmol) and 3-methylazetidin-3-ol (23 mg, 0.14 mmol) in 5 mL MeOH/DCM (1:1) was stirred at room temperature and added with acetic acid until the pH was around 4. After the reaction was stirred for 1 hour, sodium cyanoborohydride (38 mg, 0.6 mmol) was added and the reaction was stirred at room temperature for an additional 1 hour. The crude mixture was purified by column chromatography (silica gel, gradient elution, 0 to 15% MeOH/DCM). The fractions containing the product were collected and the solvent was removed under reduced pressure. The resulting oil was treated with 4M HCl in dioxane and stirred at room temperature for 30 min. After evaporation to dryness, the crude mixture was purified by Sephadex column chromatography (100% MeOH). The fractions containing the product were collected and the solvent was removed under reduced pressure, resulting in the title compound. LCMS Method B, RT=3.063 min, m/z=670.3 [M+H]⁺, LCMS Method D, RT=3.677 min, m/z=670.2802 [M+H]⁺, exact mass: 669.2718.

### Example 103: 1-(4-(4-(2-chloro-3-(6-methoxy-5-((7-oxo-2,6-diazaspiro[3.4]octan-2-yl)methyl)pyridin-2-yl)phenyl)-1H-indazol-1-yl)-2,6-dimethoxybenzyl)-3-methylazetidine-3-carboxamide

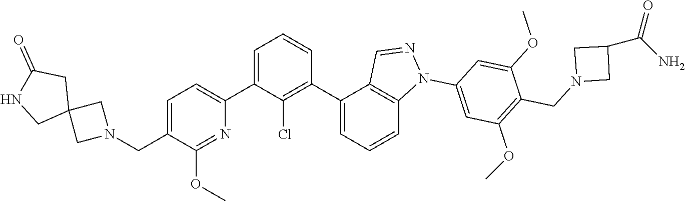 [SMILES: COc1cc(-n2ncc3c(-c4cccc(-c5ccc(CN6CC7(CNC(=O)C7)C6)c(OC)n5)c4Cl)cccc32)cc(OC)c1CN1CC(C(N)=O)C1]

A mixture of Intermediate E6 (40.0 mg, 0.063 mmol) and N-cyclopropyl-3-methylazetidine-3-carboxamide (26.34 mg, 0.189 mmol) in 4 mL MeOH/DCM (1:1) was stirred at room temperature and added with acetic acid until the pH was around 4. After the reaction was stirred for 1.5 hours, sodium cyanoborohydride (19.85 mg, 0.315 mmol) was added and the reaction was stirred at room temperature for an additional 30 min. The crude mixture was purified by column chromatography (silica gel, gradient elution, 5 to 30% MeOH/DCM with addition of 0.5% TEA). The fractions containing the product were collected and the solvent was removed under reduced pressure, resulting in the title compound. LCMS Method B, RT=3.030 min, m/z=736.3 [M+H]⁺, 758.2 [M+Na]⁺, LCMS Method D, RT=3.596 min, m/z=736.3018 [M+H]⁺, exact mass: 735.2936.

### Example 104: 1-((6-(2-chloro-3-(1-(4-((3-hydroxy-3-methylazetidin-1-yl)methyl)-3,5-dimethoxyphenyl)-1H-indazol-4-yl)phenyl)-2-methoxypyridin-3-yl)methyl)-3-methylazetidine-3-carboxamide

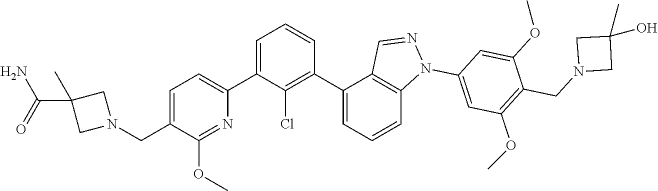 [SMILES: COc1cc(-n2ncc3c(-c4cccc(-c5ccc(CN6CC(C)(C(N)=O)C6)c(OC)n5)c4Cl)cccc32)cc(OC)c1CN1CC(C)(O)C1]

A mixture of Intermediate E9 (45 mg, 0.07 mmol) and 3-methylazetidin-3-ol (6.96 mg, 0.08 mmol) in 6 mL MeOH/CH₂Cl₂ (1:1) was stirred at room temperature and acetic acid was added until pH value around 4. After leaving for 1 hour, sodium cyanoborohydride (21.99 mg, 0.35 mmol) was added and the reaction was stirred at room temperature for an additional 1 hour. The reaction mixture was purified by Sephadex column chromatography, affording the title compound. LCMS Method B, RT=3.048 min, m/z=697.3 [M+H]⁺, LCMS Method D, RT=3.671 min, m/z=697.2890 [M+H]⁺, exact mass: 696.2827.

### Example 105: 1-(4-(4-(2-chloro-3-(5-(((cis-3-hydroxycyclobutyl)amino)methyl)-6-methoxypyridin-2-yl)phenyl)-1H-indazol-1-yl)-2,6-dimethoxybenzyl)-3-methylazetidine-3-carboxamide

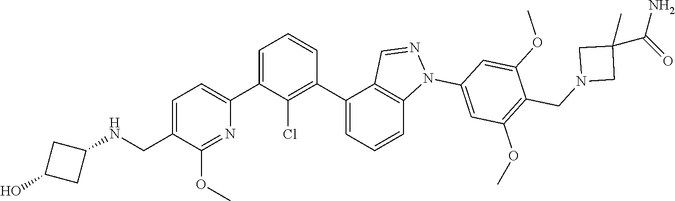 [SMILES: COc1cc(-n2ncc3c(-c4cccc(-c5ccc(CN[C@H]6C[C@@H](O)C6)c(OC)n5)c4Cl)cccc32)cc(OC)c1CN1CC(C)(C(N)=O)C1]

A mixture of Intermediate E7 (49 mg, 0.07 mmol) and 3-methylazetidine-3-carboxamide (9.13 mg, 0.08 mmol) in 6 mL MeOH/CH₂Cl₂ (1:1) was stirred at room temperature and acetic acid was added until pH value around 4. After leaving for 1 hour, sodium cyanoborohydride (21.99 mg, 0.35 mmol) was added and the reaction was stirred at room temperature for an additional 1 hour before 4M HCl in dioxane (1 mL) was added. After 30 minutes, the solvent was removed under reduced pressure and the residue was washed with dioxane. The crude mixture was purified by Sephadex column chromatography, affording the title compound. LCMS Method B, RT=3.032 min, m/z=697.3 [M+H]⁺, LCMS Method D, RT=3.813 min, m/z=697.2931 [M+H]⁺, exact mass: 696.2827.

### Example 106: cis-3-(((6-(2-chloro-3-(1-(5-(((cis-3-hydroxycyclobutyl)amino)methyl)-4-methoxypyridin-2-yl)-1H-indazol-4-yl)phenyl)-2-methoxypyridin-3-yl)methyl)amino)cyclobutanol

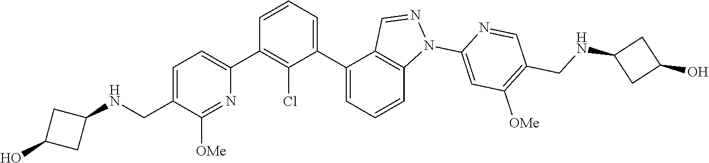 [SMILES: COc1cc(-n2ncc3c(-c4cccc(-c5ccc(CN[C@H]6C[C@@H](O)C6)c(OC)n5)c4Cl)cccc32)ncc1CN[C@H]1C[C@@H](O)C1]

A mixture of Intermediate D3 (54.0 mg, 0.108 mmol) and cis-3-aminocyclobutanol hydrochloride (32.1 mg, 0.260 mmol) in 5 mL MeOH/DCM (1:1) was stirred at room temperature and added with acetic acid until the pH was around 4. After the reaction was stirred for 1 hour, sodium cyanoborohydride (23.8 mg, 0.379 mmol) was added and the reaction was stirred at room temperature for an additional 1 hour. The crude mixture was purified by column chromatography (silica gel, gradient elution, 10 to 50% MeOH/DCM). The fractions containing the product were collected and the solvent was removed under reduced pressure, resulting in the title compound. LCMS Method B, RT=3.065 min, m/z=641.3 [M+H]⁺, LCMS Method D, RT=3.813 min, m/z=641.2617 [M+H]⁺, exact mass: 640.2565.

### Example 107: 3-((4-(4-(2-chloro-3-(6-methoxy-5-((7-oxo-2,6-diazaspiro[3.4]octan-2-yl)methyl)pyridin-2-yl)phenyl)-1H-indazol-1-yl)-2,6-dimethoxybenzyl)amino)-2,2-dimethylpropanamide

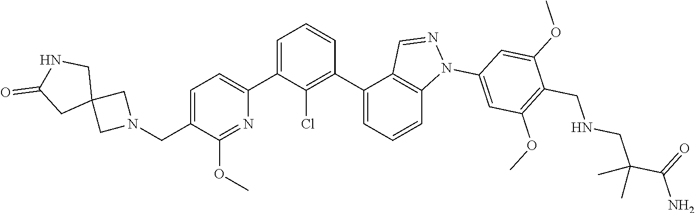 [SMILES: COc1cc(-n2ncc3c(-c4cccc(-c5ccc(CN6CC7(CNC(=O)C7)C6)c(OC)n5)c4Cl)cccc32)cc(OC)c1CNCC(C)(C)C(N)=O]

A mixture of Intermediate E6 (45 mg, 0.07 mmol) and 3-amino-2,2-dimethylpropanamide (9.28 mg, 0.08 mmol) in 6 mL MeOH/CH₂Cl₂ (1:1) was stirred at room temperature and acetic acid was added until pH value around 4. After leaving for 1 hour, sodium cyanoborohydride (21.99 mg, 0.35 mmol) was added and the reaction was stirred at room temperature for an additional 1 hour. The reaction mixture was purified by Sephadex column chromatography, affording the title compound. LCMS Method F, RT=6.677 min, m/z=738.3 [M+H]⁺, LCMS Method D, RT=3.645 min, m/z=738.3201 [M+H]⁺, exact mass: 737.3092.

### Example 108: (S)-2-((6-(2-chloro-3-(1-(4-methoxy-5-((((5-oxopyrrolidin-2-yl)methyl)amino)methyl)pyridin-2-yl)-1H-indazol-4-yl)phenyl)-2-methoxypyridin-3-yl)methyl)-2,6-diazaspiro[3.4]octan-7-one

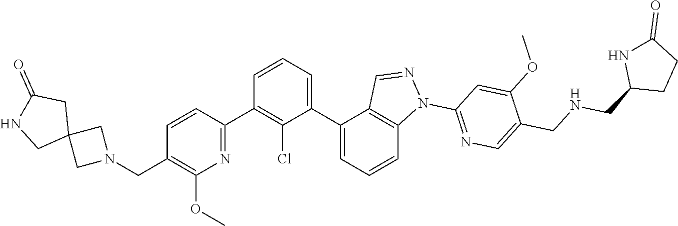 [SMILES: COc1cc(-n2ncc3c(-c4cccc(-c5ccc(CN6CC7(CNC(=O)C7)C6)c(OC)n5)c4Cl)cccc32)ncc1CNC[C@@H]1CCC(=O)N1]

A mixture of Intermediate E12 (35 mg, 0.06 mmol) and (S)-5-(aminomethyl)pyrrolidin-2-one (7 mg, 0.06 mmol) in 3 mL MeOH/DCM (1:1) was stirred at room temperature and added with acetic acid until the pH was around 4. After the reaction was stirred for 1 hour, sodium cyanoborohydride (19 mg, 0.3 mmol) was added and the reaction was stirred at room temperature for an additional 1 hour. The crude mixture was purified by column chromatography (silica gel, gradient elution, 0 to 20% MeOH/DCM). The fractions containing the product were collected and the solvent was removed under reduced pressure, resulting in the title compound. LCMS Method B, RT=2.988 min, m/z=707.3 [M+H]⁺, m/z=729.3 [M+Na]⁺, LCMS Method D, RT=3.429 min, m/z=707.2859 [M+H]⁺, exact mass: 706.2783.

### Example 109: 3-((4-(4-(2-chloro-3-(5-(((cis-3-hydroxycyclobutyl)amino)methyl)-6-methoxypyridin-2-yl)phenyl)-1H-indazol-1-yl)-2,6-dimethoxybenzyl)amino)-2,2-dimethylpropanamide

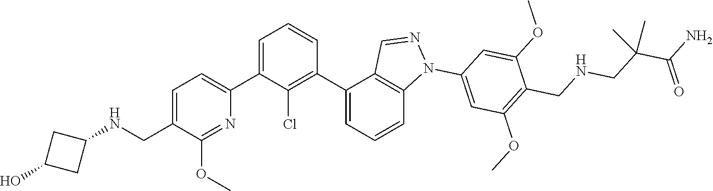 [SMILES: COc1cc(-n2ncc3c(-c4cccc(-c5ccc(CN[C@H]6C[C@@H](O)C6)c(OC)n5)c4Cl)cccc32)cc(OC)c1CNCC(C)(C)C(N)=O]

A mixture of Intermediate E7 (35.00 mg, 0.050 mmol) and 3-amino-2,2-dimethylpropanamide (17.45 mg, 0.150 mmol) in 4 mL MeOH/DCM (1:1) was stirred at room temperature and added with acetic acid until the pH was around 4. After the reaction was stirred for 2 hours, sodium cyanoborohydride (15.80 mg, 0.25 mmol) was added. The reaction was stirred at room temperature for an additional 1 hour, quenched by a small amount of MeOH and evaporated to dryness. Then, the residue was dissolved in DCM and trifluoroacetic acid (0.2 mL) was added. After stirring at room temperature for 30 minutes, the residual solvent was removed under reduced pressure. The crude mixture was purified by Sephadex column chromatography (100% MeOH), affording the title compound. LCMS method F, RT=6.779 min, m/z=699.3 [M+H]⁺, LCMS Method D, RT=3.672 min, m/z=699.3072 [M+H]⁺, exact mass: 698.2984.

### Example 110: (R)-2-((6-(2-chloro-3-(1-(5-(((2-hydroxypropyl)amino)methyl)-4-methoxypyridin-2-yl)-1H-indazol-4-yl)phenyl)-2-methoxypyridin-3-yl)methyl)-2,6-diazaspiro[3.4]octan-7-one

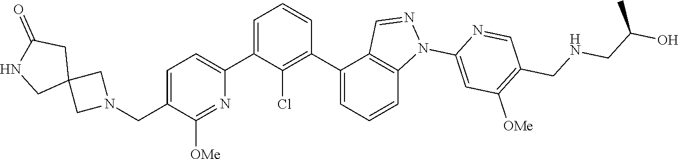 [SMILES: COc1cc(-n2ncc3c(-c4cccc(-c5ccc(CN6CC7(CNC(=O)C7)C6)c(OC)n5)c4Cl)cccc32)ncc1CNC[C@@H](C)O]

A mixture of Intermediate E12 (0.10 g, 0.17 mmol) and (R)-1-aminopropan-2-ol (0.02 g, 0.20 mmol) in 4 mL MeOH/DCM (1:1) was stirred at room temperature and acetic acid was added until the pH was around 4. After the reaction was stirred for 1 hour, sodium cyanoborohydride (0.03 g, 0.51 mmol) was added and the reaction was stirred at room temperature for an additional 1 hour. The crude mixture was purified by column chromatography (silica gel, gradient elution, 5 to 25% MeOH/DCM). The fractions containing the product were collected and the solvent was removed under reduced pressure, resulting in the title compound. LCMS Method F, RT=5.682 min, m/z=668.3 [M+H]⁺, 690.3 [M+Na]˜, LCMS Method D, RT=3.501 min, m/z=668.2784 [M+H]⁺, exact mass: 667.2674.

### Example 111: 2-((6-(2-chloro-3-(1-(5-(((cis-3-hydroxycyclobutyl)amino)methyl)-4-methoxypyridin-2-yl)-1H-indazol-4-yl)phenyl)-2-methoxypyridin-3-yl)methyl)-2,6-diazaspiro[3.4]octan-7-one

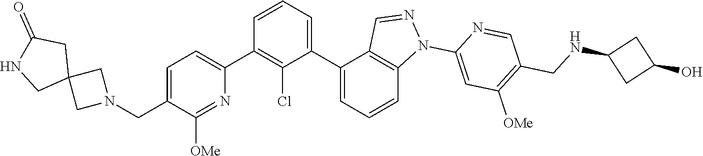 [SMILES: COc1cc(-n2ncc3c(-c4cccc(-c5ccc(CN6CC7(CNC(=O)C7)C6)c(OC)n5)c4Cl)cccc32)ncc1CN[C@H]1C[C@@H](O)C1]

A mixture of Intermediate E12 (42.5 mg, 0.070 mmol) and cis-3-aminocyclobutanol hydrochloride (10.0 mg, 0.084 mmol) in 5 mL MeOH/DCM (1:1) was stirred at room temperature and added with acetic acid until the pH was around 4. After the reaction was stirred for 1 hour, sodium cyanoborohydride (13.2 mg, 0.21 mmol) was added and the reaction was stirred at room temperature for an additional 1 hour. The crude mixture was purified by column chromatography (silica gel, gradient elution, 10 to 50% MeOH/DCM). The fractions containing the product were collected and the solvent was removed under reduced pressure, resulting in the title compound. LCMS Method F, RT=5.617 min, m/z=680.3 [M+H]⁺, LCMS Method D, RT=3.483 min, m/z=680.2797 [M+H]⁺, exact mass: 679.2674.

### Example 112: (R)-2-((6-(2-chloro-3-(1-(4-(((2-hydroxypropyl)amino)methyl)-3,5-dimethoxyphenyl)-1H-indazol-4-yl)phenyl)-2-methoxypyridin-3-yl)methyl)-2,6-diazaspiro[3.4]octan-7-one

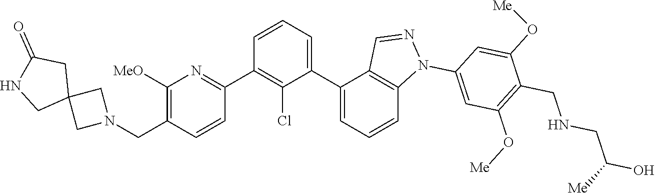 [SMILES: COc1cc(-n2ncc3c(-c4cccc(-c5ccc(CN6CC7(CNC(=O)C7)C6)c(OC)n5)c4Cl)cccc32)cc(OC)c1CNC[C@@H](C)O]

A mixture of Intermediate E6 (30.0 mg, 0.047 mmol) and (R)-1-aminopropan-2-ol (4.2 mg, 0.056 mmol) in 4 mL MeOH/DCM (1:1) was stirred at room temperature and added with acetic acid until the pH was around 4. After the reaction was stirred for 1.5 hour, sodium cyanoborohydride (14.8 mg, 0.24 mmol) was added and the reaction was stirred at room temperature for an additional 30 min. The crude mixture was purified by column chromatography (silica gel, 10% MeOH/DCM). The fractions containing the product were collected and the solvent was removed under reduced pressure, resulting in the title compound. LCMS Method F, RT=6.522 min, m/z=697.3 [M+H]⁺, LCMS Method D, RT=3.617 min, m/z=697.2920 [M+H]⁺, exact mass: 696.2827.

### Example 113: cis-3-(((6-(2-chloro-3-(1-(4-((((R)-2-hydroxypropyl)amino)methyl)-3,5-dimethoxyphenyl)-1H-indazol-4-yl)phenyl)-2-methoxypyridin-3-yl)methyl)amino)cyclobutan-1-ol

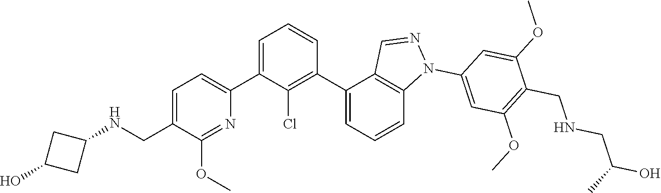 [SMILES: COc1cc(-n2ncc3c(-c4cccc(-c5ccc(CN[C@H]6C[C@@H](O)C6)c(OC)n5)c4Cl)cccc32)cc(OC)c1CNC[C@@H](C)O]

A mixture of Intermediate E7 (40.00 mg, 0.057 mmol) and (R)-(−)-1-amino-2-propanol (8.56 mg, 0.114 mmol) in 3 mL MeOH/DCM (1:1) was stirred at room temperature and added with acetic acid until the pH was around 4. After the reaction was stirred for 1 hour, sodium cyanoborohydride (18.27 mg, 0.29 mmol) was added. The reaction was stirred at room temperature for an additional 1 hour, quenched by a small amount of MeOH and evaporated to dryness. Then, the residue was dissolved in DCM and trifluoroacetic acid (0.1 mL) was added. After stirring at room temperature for 30 minutes, the residual solvent was removed under reduced pressure. The crude mixture was purified by column chromatography (silica gel, gradient elution, 5 to 30% MeOH/DCM with addition of 0.5% TEA). The fractions containing the product were collected and the solvent was removed under reduced pressure, resulting in the title compound. LCMS method B, RT=3.056 min, m/z=658.3 [M+H]⁺, LCMS Method D, RT=3.661 min, m/z=658.2802 [M+H]⁺, exact mass: 657.2718.

### Example 114: 1-(4-(4-(2-chloro-3-(5-(4,5-dihydro-1H-imidazol-2-yl)-6-methoxypyridin-2-yl)phenyl)-1H-indazol-1-yl)-2,6-dimethoxybenzyl)-3-methylazetidine-3-carboxamide

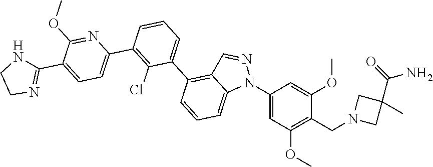 [SMILES: COc1cc(-n2ncc3c(-c4cccc(-c5ccc(C6=NCCN6)c(OC)n5)c4Cl)cccc32)cc(OC)c1CN1CC(C)(C(N)=O)C1]

NEt₃ was added to a mixture of 3-methylazetidine-3-carboxamide (8.9 mg, 0.07 mmol) in 6 mL MeOH/DCM (1:1) until the pH was around 7. Then, the mixture was transferred to Intermediate E5 (36.0 mg, 0.06 mmol) at room temperature. After that, acetic acid was added until the pH was around 4. After the reaction was stirred for 1 hour, sodium cyanoborohydride (20.0 mg, 0.32 mmol) was added and the reaction was stirred at room temperature for overnight. The reaction mixture was purified by column chromatography (silica gel, gradient elution, 0 to 50% MeOH/DCM). The fractions containing the product were collected and the solvent was removed under reduced pressure to provide the title compound. LCMS Method B, RT=3.097 min, m/z=666.3 [M+H]⁺, LCMS Method D, RT=3.665 min, m/z=666.2607 [M+H]⁺, exact mass: 665.2517.

### Example 115: 2-(4-(4-(2-chloro-3-(5-(((cis-3-hydroxycyclobutyl)amino)methyl)-6-methoxypyridin-2-yl)phenyl)-1H-indazol-1-yl)-2,6-dimethoxybenzyl)-2,6-diazaspiro[3.4]octan-7-one

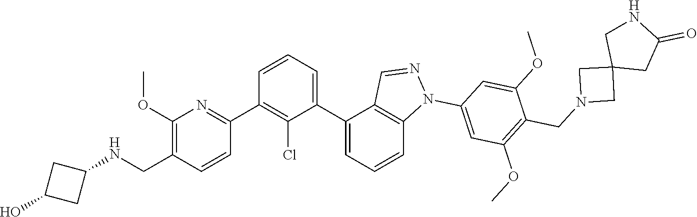 [SMILES: COc1cc(-n2ncc3c(-c4cccc(-c5ccc(CN[C@H]6C[C@@H](O)C6)c(OC)n5)c4Cl)cccc32)cc(OC)c1CN1CC2(CNC(=O)C2)C1]

A mixture of Intermediate E7 (80 mg, 0.11 mmol) and 2,6-diazaspiro[3.4]octan-7-one (19 mg, 0.15 mmol) in 10 mL MeOH/DCM (1:1) was stirred at room temperature and added with acetic acid until the pH was around 4. After the reaction was stirred for 1 hour, sodium cyanoborohydride (35 mg, 0.55 mmol) was added and the reaction was stirred at room temperature for an additional 1 hour. The crude mixture was purified by column chromatography (silica gel, gradient elution, 0 to 10% MeOH/DCM). The fractions containing the product were collected and the solvent was removed under reduced pressure. The resulting oil was treated with 4M HCl in dioxane and stirred at room temperature for 30 min. After evaporation to dryness, the crude mixture was purified by column chromatography (silica gel, gradient elution, 0 to 30% MeOH/DCM). The fractions containing the product were collected and the solvent was removed under reduced pressure, resulting the title compound. LCMS Method F, RT=6.479 min, m/z=709.3 [M+H]⁺, m/z=731.3 [M+Na]⁺, LCMS Method D, RT=3.569 min, m/z=709.2929 [M+H]⁺, exact mass: 708.2827. ¹H NMR (500 MHz, MeOD-d₄) δ ppm 8.03 (s, 1H), 7.91 (d, J=8.6 Hz, 1H), 7.74 (d, J=7.4 Hz, 11H), 7.67 (dd, J=7.3, 2.0 Hz, 1H), 7.64-7.60 (m, 1H), 7.56 (t, J=7.5 Hz, 1H), 7.53 (dd, J=7.6, 2.1 Hz, 1H), 7.28 (dd, J=7.3, 4.5 Hz, 2H), 7.06 (s, 2H), 5.22 (s, 2H), 4.64 (s, 2H), 4.05 (s, 3H), 3.95 (s, 6H), 3.93 (s, 2H), 3.80 (s, 2H), 3.56 (d, J=4.4 Hz, 6H), 2.56 (s, 2H), 1.30-1.27 (s, 4H).

### Example 116: 2-((6-(2-chloro-3-(1-(5-(((2-hydroxyethyl)amino)methyl)-4-methoxypyridin-2-yl)-1H-indazol-4-yl)phenyl)-2-methoxypyridin-3-yl)methyl)-2,5-diazaspiro[3.4]octan-6-one

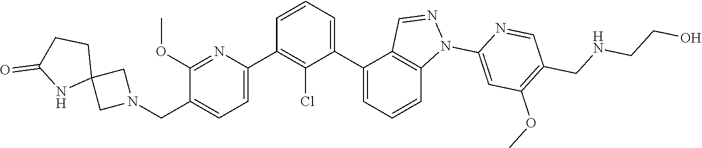 [SMILES: COc1cc(-n2ncc3c(-c4cccc(-c5ccc(CN6CC7(CCC(=O)N7)C6)c(OC)n5)c4Cl)cccc32)ncc1CNCCO]

### Step 1: 6-(4-(2-chloro-3-(6-methoxy-5-((6-oxo-2,5-diazaspiro[3.4]octan-2-yl)methyl)pyridin-2-yl)phenyl)-1H-indazol-1-yl)-4-methoxynicotinaldehyde

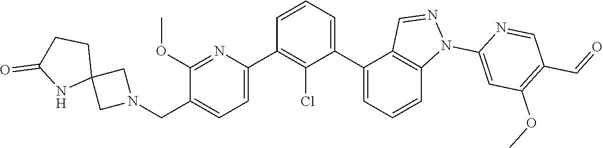 [SMILES: COc1cc(-n2ncc3c(-c4cccc(-c5ccc(CN6CC7(CCC(=O)N7)C6)c(OC)n5)c4Cl)cccc32)ncc1C=O]

A mixture of Intermediate C1-11 (90.0 mg, 0.19 mmol), Intermediate B5-1 (102.8 mg, 0.27 mmol), Pd(PPh₃)₄ (26.5 mg, 0.02 mmol) and K₂CO₇ (93.5 mg, 0.68 mmol) in a mixture of 1,4-dioxane and H₂O (6:1) 7 mL was sparged with argon for 10 min before stirring at 95° C. for 2 h under argon atmosphere. Then, the mixture was cooled down and diluted with brine. The resulting mixture was extracted 3 times with EtOAc and the organic layer was collected, dried over anhydrous Na₂SO₄, filtered, and evaporated to dryness. The crude mixture was purified by column chromatography (silica gel, gradient elution, 0 to 10% MeOH/DCM) to provide the title compound (70.0 mg). LCMS Method B, RT=3.444 min, m/z=609.2 [M+H]⁺, exact mass: 608.1939.

### Step 2: 2-((6-(2-chloro-3-(1-(5-(((2-hydroxyethyl)amino)methyl)-4-methoxypyridin-2-yl)-1H-indazol-4-yl)phenyl)-2-methoxypyridin-3-yl)methyl)-2,5-diazaspiro[3.4]octan-6-one

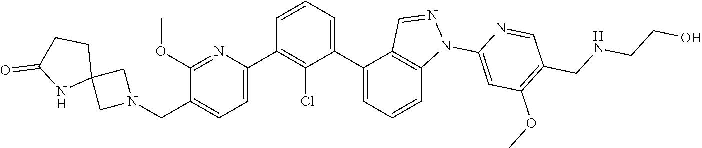 [SMILES: COc1cc(-n2ncc3c(-c4cccc(-c5ccc(CN6CC7(CCC(=O)N7)C6)c(OC)n5)c4Cl)cccc32)ncc1CNCCO]

A mixture of 6-(4-(2-chloro-3-(6-methoxy-5-((6-oxo-2,5-diazaspiro[3.4]octan-2-yl)methyl)pyridin-2-yl)phenyl)-1H-indazol-1-yl)-4-methoxynicotinaldehyde (70.0 mg, 0.11 mmol) and ethanolamine (0.01 mL, 0.17 mmol) in 3 mL MeOH/DCM (1:1) was stirred at room temperature and added with acetic acid until the pH was around 4. After the reaction was stirred for 2 hours, sodium cyanoborohydride (38.0 mg, 0.60 mmol) was added and the reaction was stirred at room temperature for 1.5 hours. The crude mixture was purified by Sephadex column chromatography (100% MeOH). The fractions containing the product were collected and the solvent was removed under reduced pressure to provide the title compound. LCMS Method B, RT=2.964 min, m/z=654.3 [M+H]⁺, LCMS Method D, RT=3.445 min, m/z=654.2558 [M+H]⁺, exact mass: 653.2517.

### Example 117: (R)-1-(4-(4-(2-chloro-3-(6-methoxy-5-(((((S)-5-oxopyrrolidin-2-yl)methyl)amino)methyl)pyridin-2-yl)phenyl)-1H-indazol-1-yl)-2,6-dimethoxybenzyl)pyrrolidine-3-carboxylic acid

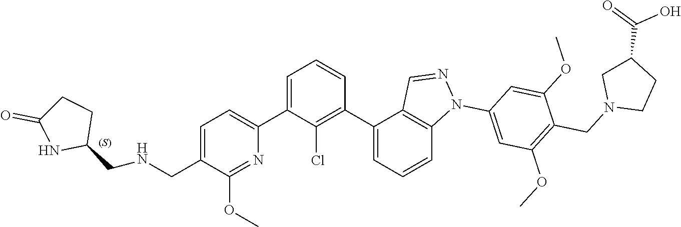

A mixture of Intermediate E3 (35.00 mg, 0.048 mmol) and (R)-pyrrolidine-3-carboxylic acid (11.04 mg, 0.096 mmol) in 4 mL MeOH/DCM (1:1) was stirred at room temperature and added with acetic acid until the pH was around 4. After the reaction was stirred for 1 hour, sodium cyanoborohydride (15.12 mg, 0.24 mmol) was added. The reaction was stirred at room temperature for an additional 1 hour, quenched by a small amount of MeOH and evaporated to dryness. Then, the residue was dissolved in DCM and trifluoroacetic acid (0.2 mL) was added. After stirring at room temperature for 30 minutes, the residual solvent was removed under reduced pressure. The crude mixture was purified by column chromatography (silica gel, gradient elution, 5 to 50% MeOH/DCM with addition of 0.5% TEA). The fractions containing the product were collected and the solvent was removed under reduced pressure, resulting in the title compound. LCMS method B, RT=3.048 min, m/z=725.4 [M+H]⁺, LCMS Method D, RT=3.643 min, m/z=725.2844 [M+H]⁺, exact mass: 724.2776.

### Example 118: (S)-2-(((6-(4-(2-chloro-3-(5-(((cis-3-hydroxycyclobutyl)amino)methyl)-6-methoxypyridin-2-yl)phenyl)-1H-indazol-1-yl)-4-methoxypyridin-3-yl)methyl)amino)-3-hydroxypropanamide

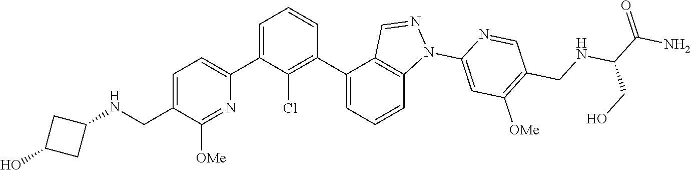 [SMILES: COc1cc(-n2ncc3c(-c4cccc(-c5ccc(CN[C@H]6C[C@@H](O)C6)c(OC)n5)c4Cl)cccc32)ncc1CN[C@@H](CO)C(N)=O]

A mixture of Intermediate E13 (0.11 g, 0.17 mmol) and (S)-2-amino-3-hydroxypropanamide (0.02 g, 0.20 mmol) in 4 mL MeOH/DCM (1:1) was stirred at room temperature and acetic acid was added until the pH was around 4. After the reaction was stirred for 1 hour, sodium cyanoborohydride (0.03 g, 0.51 mmol) was added and the reaction was stirred at room temperature for an additional 1 hour. After evaporation to dryness, the resulting crude mixture was stirred at room temperature in the presence of 4M HCl in dioxane for 30 min. All solvent was evaporated under vacuum and the crude mixture was purified by column chromatography (silica gel, gradient elution, 5 to 25% MeOH/DCM). The fractions containing the product were collected and the solvent was removed under reduced pressure, resulting in the title compound. LCMS Method G, RT=5.916 min, m/z=658.3 [M+H]⁺, LCMS Method D, RT=3.580 min, m/z=658.2526 [M+H]⁺, exact mass: 657.2466.

### Example 119: cis-3-(((6-(2-chloro-3-(1-(4-((((S)-1-hydroxypropan-2-yl)amino)methyl)-3,5-dimethoxyphenyl)-1H-indazol-4-yl)phenyl)-2-methoxypyridin-3-yl)methyl)amino)cyclobutanol

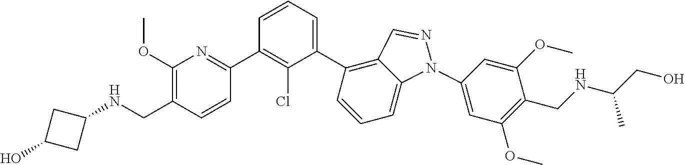 [SMILES: COc1cc(-n2ncc3c(-c4cccc(-c5ccc(CN[C@H]6C[C@@H](O)C6)c(OC)n5)c4Cl)cccc32)cc(OC)c1CN[C@@H](C)CO]

A mixture of Intermediate E7 (0.02 g, 0.03 mmol) and (2S)-2-aminopropan-1-ol (2.58 mg, 0.04 mmol) in 4 mL MeOH/DCM (1:1) was stirred at room temperature and acetic acid was added until the pH was around 4. After the reaction was stirred for 1 hour, sodium cyanoborohydride (9.00 mg, 0.154 mmol) was added and the reaction was stirred at room temperature for an additional 1 hour. After evaporation to dryness, the resulting crude mixture was stirred at room temperature in the presence of 4M HCl in dioxane for 30 min. All solvent was evaporated under vacuum and the crude mixture was purified by column chromatography (silica gel, gradient elution, 5 to 50% MeOH/DCM). The fractions containing the product were collected and the solvent was removed under reduced pressure, resulting the title compound. LCMS Method B, RT=3.055 min, m/z=658.3 [M+H]⁺, LCMS Method D, RT=3.860 min, m/z=658.2800 [M+H]⁺, exact mass: 657.2718. ¹H NMR (500 MHz, MeOD-d₄) S 8.02 (s, 1H), 7.90 (d, J=7.7 Hz, 1H), 7.73 (d, J=7.5 Hz, 1H), 7.66-7.59 (m, 2H), 7.56-7.49 (m, 2H), 7.29-7.23 (m, 2H), 7.09 (s, 2H), 4.25 (s, 2H), 4.02 (s, 3H), 3.98 (s, 6H), 3.94-3.89 (m, 1H), 3.78 (s, 2H), 3.75-3.71 (m, 1H), 3.17-3.07 (m, 2H), 2.93-2.84 (m, 1H), 1.26 (m, 7H).

### Example 120: ((6-(4-(2-chloro-3-(5-(((cis-3-hydroxycyclobutyl)amino)methyl)-6-methoxypyridin-2-yl)phenyl)-1H-indazol-1-yl)-4-methoxypyridin-3-yl)methyl)-D-serine

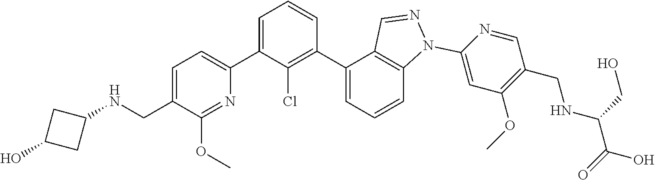

A mixture of Intermediate E13 (30.00 mg, 0.045 mmol) and D-serine (11.9 mg, 0.113 mmol) in 3 mL H₂O/MeOH/DCM (1:1:1) was stirred at room temperature and added with acetic acid until the pH was around 4. After the reaction was stirred for 1 hour, sodium cyanoborohydride (14.17 mg, 0.225 mmol) was added. The reaction was stirred at room temperature for an additional 1 hour, quenched by a small amount of MeOH and evaporated to dryness. Then, the residue was dissolved in DCM and trifluoroacetic acid (0.1 mL) was added. After stirring at room temperature for 30 minutes, the residual solvent was removed under reduced pressure. The crude mixture was purified by column chromatography (silica gel, gradient elution, 5 to 60% MeOH/DCM with addition of 0.5% TEA). The fractions containing the product were collected and the solvent was removed under reduced pressure, resulting in the title compound. LCMS Method F, RT=5.827 min, m/z=659.4 [M+H]⁺, LCMS Method D, RT=3.589 min, m/z=659.2391 [M+H]⁺, exact mass: 658.2307.

### Example 121: (S)-2-((4-(4-(2-chloro-3-(5-(((cis-3-hydroxycyclobutyl)amino)methyl)-6-methoxypyridin-2-yl)phenyl)-1H-indazol-1-yl)-2,6-dimethoxybenzyl)amino)-3-hydroxypropanamide

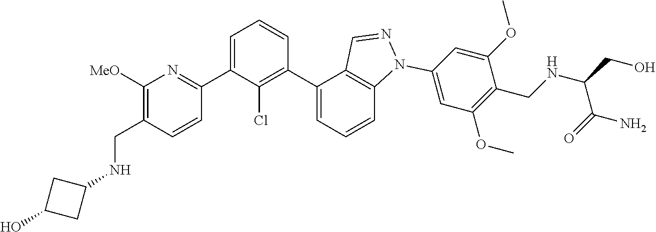

A mixture of Intermediate E7 (50 mg, 0.072 mmol) and 3-methylazetidin-3-ol hydrochloride (9 mg, 0.09 mmol) in 2 mL MeOH/DCM (1:1) was stirred at room temperature and acetic acid was added until the pH was around 4. After the reaction was stirred for 1 hour, sodium cyanoborohydride (23 mg, 0.36 mmol) was added and the reaction was stirred at room temperature for an additional 1 hour. Then, the reaction was quenched by addition of 10 mL MeOH and the solvent was removed under reduced pressure. Then, the residue was dissolved in 1 mL DCM/trifluoroacetic acid (1:1). After stirring at room temperature for 30 minutes, the residual solvent was removed under reduced pressure. The crude mixture was purified by column chromatography (silica gel, gradient elution, 5 to 10% MeOH/DCM with addition of 1% TEA). The fractions containing the product were collected and the solvent was removed under reduced pressure, resulting in the title compound. LCMS Method F, RT=6.607 min, m/z=709.3 [M+Na]⁺, LCMS Method D, RT=3.544 min, m/z=687.2700 [M+H]⁺, exact mass: 686.2620.

### Example 122: cis-3-(((6-(2-chloro-3-(1-(5-((((R)-2-hydroxypropyl)amino)methyl)-4-methoxypyridin-2-yl)-1H-indazol-4-yl)phenyl)-2-methoxypyridin-3-yl)methyl)amino)cyclobutanol

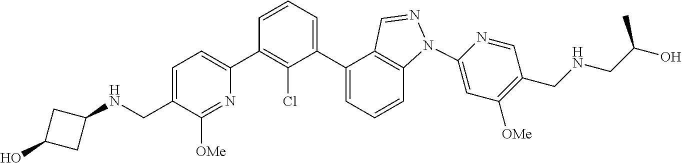 [SMILES: COc1cc(-n2ncc3c(-c4cccc(-c5ccc(CN[C@H]6C[C@@H](O)C6)c(OC)n5)c4Cl)cccc32)ncc1CNC[C@@H](C)O]

A mixture of Intermediate E13 (0.12 g, 0.17 mmol) and N-cyclopropyl-3-methylazetidine-3-carboxamide (0.03 g, 0.20 mmol) in 4 mL MeOH/DCM (1:1) was stirred at room temperature and acetic acid was added until the pH was around 4. After the reaction was stirred for 1 hour, sodium cyanoborohydride (0.03 g, 0.51 mmol) was added and the reaction was stirred at room temperature for an additional 1 hour. After evaporation to dryness, the resulting crude mixture was stirred at room temperature in the presence of 4M HCl in dioxane for 30 min. All solvent was evaporated under vacuum and the crude mixture was purified by column chromatography (silica gel, gradient elution, 5 to 25% MeOH/DCM). The fractions containing the product were collected and the solvent was removed under reduced pressure, resulting in the title compound. LCMS Method F, RT=6.772 min, m/z=629.3 [M+H]⁺, LCMS Method D, RT=3.763 min, m/z=629.2586 [M+H]⁺, exact mass: 628.2565.

### Example 123: (4-(4-(2-chloro-3-(6-methoxy-5-(((((S)-5-oxopyrrolidin-2-yl)methyl)amino)methyl)pyridin-2-yl)phenyl)-1H-indazol-1-yl)-2,6-dimethoxybenzyl)-D-serine

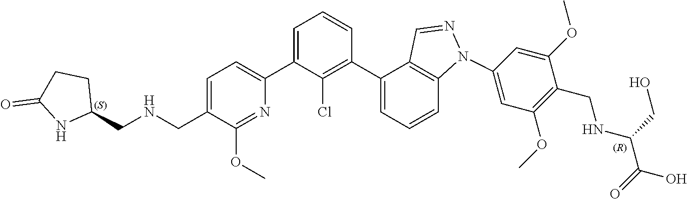

A mixture of Intermediate E3 (50 mg, 0.07 mmol) and D-serine (8.40 mg, 0.08 mmol) in 6 mL H₂O/1,4-dioxane (1:1) was stirred at room temperature and acetic acid was added until getting pH value around 4. After leaving for 1 hour, sodium cyanoborohydride (21.99 mg, 0.35 mmol) was added and the reaction was stirred at room temperature for an additional 1 hour before 4M HCl in dioxane (1 mL) was added. After 30 minutes, the solvent was removed under reduced pressure and the residue was washed with dioxane. The reaction mixture was purified by Sephadex column chromatography, affording the title compound. LCMS Method F, RT=6.145 min, m/z=715.3 [M+H]⁺, LCMS Method D, RT=3.668 min, m/z=715.2600 [M+H]⁺, exact mass: 714.2569.

### Example 124: 1-((6-(4-(2-chloro-(5-(((cis-3-hydroxycyclobutyl)amino)methyl)-6-methoxypyridin-2-yl)phenyl)-1H-indazol-1-yl)-4-methoxypyridin-3-yl)methyl)-3-methylazetidin-3-ol

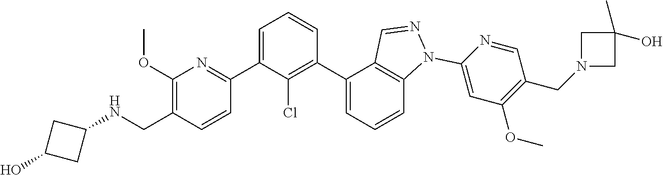 [SMILES: COc1cc(-n2ncc3c(-c4cccc(-c5ccc(CN[C@H]6C[C@@H](O)C6)c(OC)n5)c4Cl)cccc32)ncc1CN1CC(C)(O)C1]

A mixture of Intermediate E13 (83 mg, 0.12 mmol) and 3-methylazetidin-3-ol (13 mg, 0.15 mmol) in 8 mL MeOH/DCM (1:1) was stirred at room temperature and added with acetic acid until the pH was around 4. After the reaction was stirred for 1 hour, sodium cyanoborohydride (39 mg, 0.62 mmol) was added and the reaction was stirred at room temperature for an additional 1 hour. The crude mixture was purified by column chromatography (silica gel, gradient elution, 0 to 10% MeOH/EtOAc). The fractions containing the product were collected and the solvent was removed under reduced pressure. The resulting oil was treated with 4M HCl in dioxane and stirred at room temperature for 30 min. After evaporation to dryness, the crude mixture was purified by Sephadex column chromatography (100% MeOH). The fractions containing the product were collected and the solvent was removed under reduced pressure, resulting in the title compound. LCMS Method F, RT=6.036 min, m/z=641.3 [M+H]⁺, LCMS Method D, RT=3.786 min, m/z=641.2631 [M+H]⁺, exact mass: 640.2565.

### Example 125: cis-3-(((6-(2-chloro-3-(1-(4-(((2-hydroxyethyl)amino)methyl)-3,5-dimethoxyphenyl)-1H-indazol-4-yl)phenyl)-2-methoxypyridin-3-yl)methyl)amino)cyclobutanol

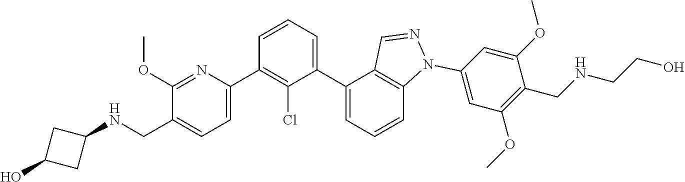 [SMILES: COc1cc(-n2ncc3c(-c4cccc(-c5ccc(CN[C@H]6C[C@@H](O)C6)c(OC)n5)c4Cl)cccc32)cc(OC)c1CNCCO]

A mixture of Intermediate E7 (20.0 mg, 0.03 mmol) and ethanolamine (4.0 μL, 0.07 mmol) in 2 mL MeOH/DCM (1:1) was stirred at room temperature and added with acetic acid until the pH was around 4. After the reaction was stirred for 2 hours, sodium cyanoborohydride (11.0 mg, 0.18 mmol) was added and the reaction was stirred at room temperature for 18 hours. The crude mixture was purified by Sephadex column chromatography (100% MeOH). The fractions containing the product were collected and the solvent was removed under reduced pressure to provide the title compound. LCMS Method B, RT=3.048 min, m/z=644.3 [M+H]⁺, LCMS Method D, RT=3.571 min, m/z=644.2644 [M+H]⁺, exact mass: 643.2561.

### Example 126: (4-(4-(2-chloro-3-(5-(((cis-3-hydroxycyclobutyl)amino)methyl)-6-methoxypyridin-2-yl)phenyl)-1H-indazol-1-yl)-2,6-dimethoxybenzyl)-D-serine

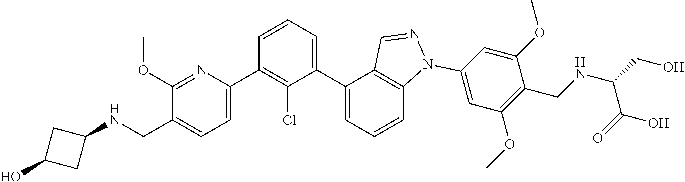 [SMILES: COc1cc(-n2ncc3c(-c4cccc(-c5ccc(CN[C@H]6C[C@@H](O)C6)c(OC)n5)c4Cl)cccc32)cc(OC)c1CN[C@H](CO)C(=O)O]

A mixture of Intermediate E7 (20.0 mg, 0.03 mmol) and (R)-2-amino-3-hydroxypropanoic acid (5.5 mg, 0.05 mmol) in 2 mL MeOH/DCM (1:1) was stirred at room temperature and added with acetic acid until the pH was around 4. After the reaction was stirred for 2 hours, sodium cyanoborohydride (11.0 mg, 0.18 mmol) was added and the reaction was stirred at room temperature for 18 hours. The crude mixture was purified by Sephadex column chromatography (100% MeOH). The fractions containing the product were collected and the solvent was removed under reduced pressure to provide the title compound. LCMS Method B, RT=3.063 min, m/z=688.4 [M+H]⁺, LCMS Method D, RT=3.632 min, m/z=688.2541 [M+H]⁺, exact mass. 687.2460.

### Example 127: (R)-1-(((6-(2-chloro-3-(1-(4-((((R)-2-hydroxypropyl)amino)methyl)-3,5-dimethoxyphenyl)-1H-indazol-4-yl)phenyl)-2-methoxypyridin-3-yl)methyl)amino)propan-2-ol

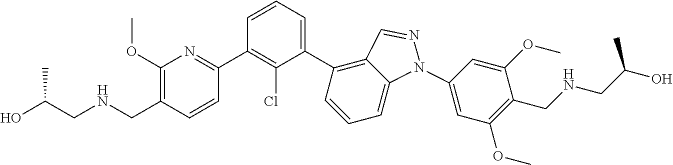 [SMILES: COc1cc(-n2ncc3c(-c4cccc(-c5ccc(CNC[C@@H](C)O)c(OC)n5)c4Cl)cccc32)cc(OC)c1CNC[C@@H](C)O]

A mixture of Intermediate D2 (40.0 mg, 0.07 mmol) and (2R)-1-aminopropan-2-ol (11.4 mg, 0.15 mmol) in 2 mL MeOH/DCM (1:1) was stirred at room temperature and acetic acid was added until the pH was around 4. After the reaction was stirred for 1 hour, sodium cyanoborohydride (47.6 mg, 0.76 mmol) was added and the reaction was stirred at room temperature for an additional 1 hour. The crude mixture was purified by column chromatography (silica gel, gradient elution, 0 to 50% MeOH/DCM). The fractions containing the product were collected and the solvent was removed under reduced pressure, resulting the title compound. LCMS Method G, RT=7.598 min, m/z=646.3 [M+H]⁺, LCMS Method D, RT=3.594 min, m/z=646.2818 [M+H]⁺, exact mass: 645.2718 ¹H NMR (500 MHz, MeOD-d4) δ 8.03 (s, 1H), 7.87 (dd, J=7.7, 3.6 Hz, 1H), 7.70 (dd, J=7.0, 4.4 Hz, 1H), 7.67-7.63 (m, 1H), 7.61-7.57 (m, 1H), 7.53-7.48 (m, 2H), 7.25 (dt, J=9.9, 4.8 Hz, 2H), 7.03 (s, 2H), 4.01 (s, 3H), 3.93 (s, 6H), 3.92 (s, 4H), 3.20-3.05 (m, 2H), 2.70-2.42 (m, 3H), 1.14 (d, J=6.5 Hz, 6H).

### Example 128: 2-((6-(2-chloro-3-(1-(4-(((2-hydroxyethyl)amino)methyl)-3,5-dimethoxyphenyl)-1H-indazol-4-yl)phenyl)-2-methoxypyridin-3-yl)methyl)-2,6-diazaspiro[3.4]octan-7-one

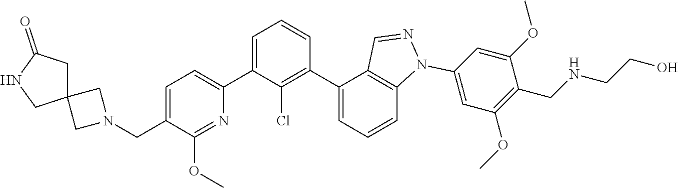 [SMILES: COc1cc(-n2ncc3c(-c4cccc(-c5ccc(CN6CC7(CNC(=O)C7)C6)c(OC)n5)c4Cl)cccc32)cc(OC)c1CNCCO]

A mixture of Intermediate E6 (35.0 mg, 0.055 mmol) and ethanolamine (6.63 mL, 0.110 mmol) in 3 mL MeOH/DCM (1:1) was stirred at room temperature and added with acetic acid until the pH was around 4. After the reaction was stirred for 0.5 hour, sodium cyanoborohydride (17.2 mg, 0.27 mmol) was added and the reaction was stirred at room temperature for an additional 30 min. The crude mixture was purified by column chromatography (silica gel, gradient elution, 5 to 25% MeOH/DCM with addition of 0.5% TEA). The fractions containing the product were collected and the solvent was removed under reduced pressure, resulting in the title compound. LCMS Method F, RT=6.055 min, m/z=683.3 [M+H]⁺, LCMS Method D, RT=3.391 min, m/z=683.2737 [M+H]⁺, exact mass: 682.2671.

### Example 129: 2-(((6-(2-chloro-3-(1-(4-(((2-hydroxyethyl)amino)methyl)-3,5-dimethoxyphenyl)-1H-indazol-4-yl)phenyl)-2-methoxypyridin-3-yl)methyl)amino)ethanol

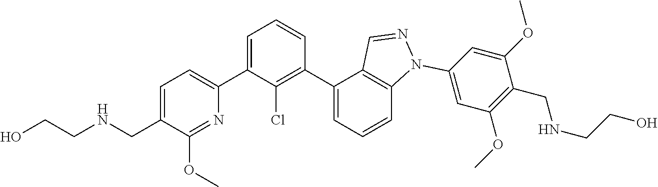 [SMILES: COc1cc(-n2ncc3c(-c4cccc(-c5ccc(CNCCO)c(OC)n5)c4Cl)cccc32)cc(OC)c1CNCCO]

A mixture of Intermediate D2 (37 mg, 0.07 mmol) and ethanolamine (9.77 mg, 0.16 mmol) in 6 mL MeOH/CH₂Cl₂ (1:1) was stirred at room temperature and acetic acid was added until getting pH value around 4. After leaving for 1 hour, sodium cyanoborohydride (21.99 mg, 0.35 mmol) was added and the reaction was stirred at room temperature for an additional 1 hour. The reaction mixture was purified by Sephadex column chromatography, affording the title compound. LCMS Method D, RT=3.500 min, m/z=618.2454 [M+H]⁺, exact mass: 617.2405.

### Example 130: (R)-1-((6-(2-chloro-3-(1-(3,5-dimethoxy-4-((7-oxo-2,6-diazaspiro[3.4]octan-2-yl)methyl)phenyl)-1H-indazol-4-yl)phenyl)-2-methoxypyridin-3-yl)methyl)pyrrolidine-3-carboxylic acid

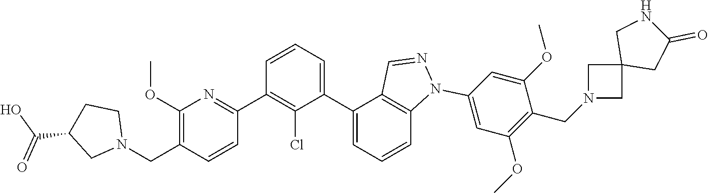 [SMILES: COc1cc(-n2ncc3c(-c4cccc(-c5ccc(CN6CC[C@@H](C(=O)O)C6)c(OC)n5)c4Cl)cccc32)cc(OC)c1CN1CC2(CNC(=O)C2)C1]

### Step J: 2-(4-(4-bromo-1H-indazol-1-yl)-2,6-dimethoxybenzyl)-2,6-diazaspiro[3.4]octan-7-one

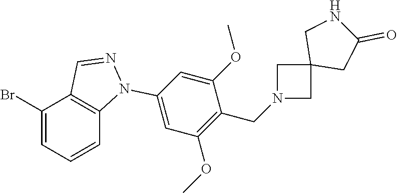 [SMILES: COc1cc(-n2ncc3c(Br)cccc32)cc(OC)c1CN1CC2(CNC(=O)C2)C1]

A mixture of Intermediate B4 (300 mg, 0.83 mmol) and 2,6-diazaspiro[3.4]octan-7-one (136 mg, 1.08 mmol) in 30 mL MeOH/DCM (1:1) was stirred at room temperature and added with acetic acid until the pH was around 4. After the reaction was stirred for 1 hour, sodium cyanoborohydride (261 mg, 4.15 mmol) was added and the reaction was stirred at room temperature for an additional 1 hour. The crude mixture was purified by column chromatography (silica gel, gradient elution, 0 to 5% MeOH/DCM).

### Step 2: 6-(2-chloro-3-(1-(3,5-dimethoxy-4-((7-oxo-2,6-diazaspiro[3.4]octan-2-yl)methyl)phenyl)-1H-indazol-4-yl)phenyl)-2-methoxynicotinaldehyde

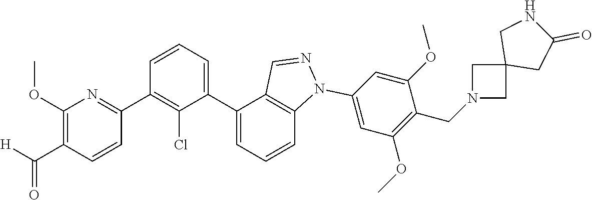 [SMILES: COc1cc(-n2ncc3c(-c4cccc(-c5ccc(C=O)c(OC)n5)c4Cl)cccc32)cc(OC)c1CN1CC2(CNC(=O)C2)C1]

A mixture of Intermediate C2 (227 mg, 0.61 mmol), 2-(4-(4-bromo-1H-indazol-1-yl)-2,6-dimethoxybenzyl)-2,6-diazaspiro[3.4]octan-7-one (220 mg, 0.47 mmol), Pd(PPh₃)₄ (54 mg, 0.05 mmol) and K₂CO₃ (195 mg, 1.41 mmol) in a mixture of 1,4-dioxane and H₂O (6:1) 25 mL was sparged with argon for 10 min before stirring at 95° C. for 3 h under argon atmosphere. Then, the mixture was cooled down and diluted with brine. The resulting mixture was extracted 3 times with EtOAc and the organic layer was collected, dried over anhydrous Na₂SO₄, filtered, and evaporated to dryness. The crude mixture was purified by column chromatography (silica gel, gradient elution, 0 to 10% MeOH/DCM).

### Step 3: (R)-1-((6-(2-chloro-3-(1-(3,5-dimethoxy-4-((7-oxo-2,6-diazaspiro[3.4]octan-2-yl)methyl)phenyl)-1H-indazol-4-yl)phenyl)-2-methoxypyridin-3-yl)methyl)pyrrolidine-3-carboxylic acid

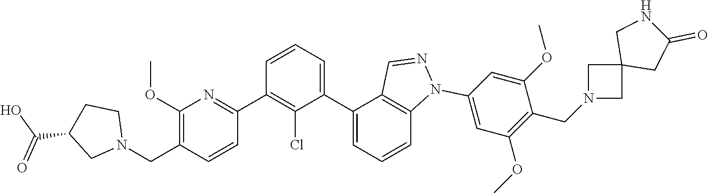 [SMILES: COc1cc(-n2ncc3c(-c4cccc(-c5ccc(CN6CC[C@@H](C(=O)O)C6)c(OC)n5)c4Cl)cccc32)cc(OC)c1CN1CC2(CNC(=O)C2)C1]

A mixture of 6-(2-chloro-3-(1-(3,5-dimethoxy-4-((7-oxo-2,6-diazaspiro[3.4]octan-2-yl)methyl)phenyl)-1H-indazol-4-yl)phenyl)-2-methoxynicotinaldehyde (50 mg, 0.08 mmol) and (R)-pyrrolidine-3-carboxylic acid (28 mg, 0.12 mmol) in 6 mL MeOH/DCM (1:1) was stirred at room temperature and added with acetic acid until the pH was around 4. After the reaction was stirred for 1 hour, sodium cyanoborohydride (39 mg, 0.62 mmol) was added and the reaction was stirred at room temperature for an additional 1 hour. The crude mixture was purified by column chromatography (silica gel, gradient elution, 0 to 20% MeOH/DCM). The fractions containing the product were collected and the solvent was removed under reduced pressure, resulting the title compound. LCMS Method G, RT=7.030 min, m/z=737.3 [M+H]⁺, m/z=759.3 [M+Na]⁺, LCMS Method D, RT=3.570 min, m/z=737.2863 [M+H]⁺, exact mass. 736.2776.

### Example 131: 1-(((4-(4-(2-chloro-3-(5-(((cis-3-hydroxycyclobutyl)amino)methyl)-6-methoxypyridin-2-yl)phenyl)-1H-indazol-1-yl)-2,6-dimethoxybenzyl)amino)methyl)cyclopropane-1-carboxamide

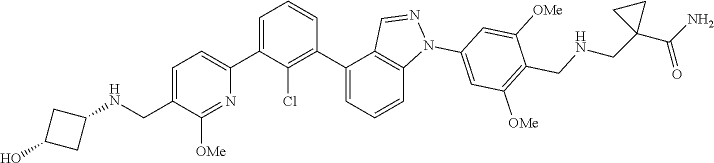 [SMILES: COc1cc(-n2ncc3c(-c4cccc(-c5ccc(CN[C@H]6C[C@@H](O)C6)c(OC)n5)c4Cl)cccc32)cc(OC)c1CNCC1(C(N)=O)CC1]

A mixture of Intermediate E7 (0.12 g, 0.17 mmol) and 1-(aminomethyl)cyclopropane-1-carboxamide (0.02 g, 0.20 mmol) in 4 mL MeOH/DCM (1:1) was stirred at room temperature and acetic acid was added until the pH was around 4. After the reaction was stirred for 1 hour, sodium cyanoborohydride (0.03 g, 0.51 mmol) was added and the reaction was stirred at room temperature for an additional 1 hour. After evaporation to dryness, the resulting crude mixture was stirred at room temperature in the presence of 4M HCl in dioxane for 30 min. All solvent was evaporated under vacuum and the crude mixture was purified by column chromatography (silica gel, gradient elution, 5 to 25% MeOH/DCM). The fractions containing the product were collected and the solvent was removed under reduced pressure, resulting in the title compound. LCMS Method G, RT=7.743 min, m/z=697.3 [M+H]⁺, LCMS Method D, RT=3.597 min, m/z=697.2866 [M+H]⁺, exact mass: 696.2827.

### Example 132: 2-((6-(2-chloro-3-(1-(5-(((2-hydroxyethyl)amino)methyl)-4-methoxypyridin-2-yl)-1H-indazol-4-yl)phenyl)-2-methoxypyridin-3-yl)methyl)-2,6-diazaspiro[3.4]octan-7-one

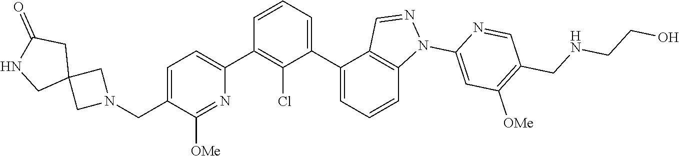 [SMILES: COc1cc(-n2ncc3c(-c4cccc(-c5ccc(CN6CC7(CNC(=O)C7)C6)c(OC)n5)c4Cl)cccc32)ncc1CNCCO]

A mixture of Intermediate E12 (80.00 mg, 0.131 mmol) and ethanolamine (11.9 mL, 0.197 mmol) in 5 mL MeOH/DCM (1:1) was stirred at room temperature and added with acetic acid until the pH was around 4. After the reaction was stirred for 1 hour, sodium cyanoborohydride (24.8 mg, 0.394 mmol) was added and the reaction was stirred at room temperature for an additional 1 hour. The crude mixture was purified by column chromatography (silica gel, gradient elution, 10 to 50% MeOH/DCM). The fractions containing the product were collected and the solvent was removed under reduced pressure, resulting the title compound. LCMS Method F, RT=6.169 min, m/z=654.3 [M+H]⁺, 678.3 [M+Na]⁺, LCMS Method D, RT=3.383 min, m/z=654.2604 [M+H]⁺, exact mass: 653.2517. ¹H NMR (500 MHz, MeOD-d₄) δ ppm 8.84 (d, J=8.6 Hz, 1H), 8.38 (s, 1H), 8.03 (s, 1H), 7.71 (s, 1H), 7.68 (d, J=7.5 Hz, 2H), 7.62 (ddd, J=15.8, 7.9, 4.7 Hz, 2H), 7.55-7.48 (m, 2H), 7.26 (dd, J=20.0, 7.3 Hz, 2H), 4.09 (s, 2H), 4.06 (s, 2H), 3.99 (s, 3H), 3.76 (t, J=5.3 Hz, 2H), 3.71 (s, 2H), 3.56 (s, 2H), 3.42 (brs, 2H), 2.99 (t, J=5.0 Hz, 2H), 2.93 (s, 3H), 2.56 (s, 2H).

### Example 133: (R)-2-((6-(2-chloro-3-(1-(4-((3-hydroxypyrrolidin-1-yl)methyl)-3,5-dimethoxyphenyl)-1H-indazol-4-yl)phenyl)-2-methoxypyridin-3-yl)methyl)-2,6-diazaspiro[3.4]octan-7-one

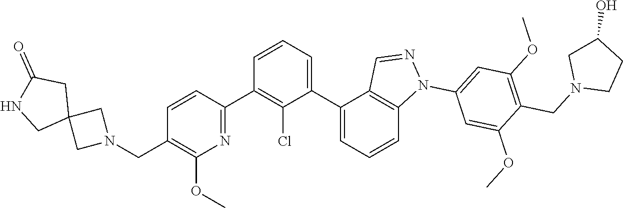 [SMILES: COc1cc(-n2ncc3c(-c4cccc(-c5ccc(CN6CC7(CNC(=O)C7)C6)c(OC)n5)c4Cl)cccc32)cc(OC)c1CN1CC[C@@H](O)C1]

A mixture of Intermediate E6 (16 mg, 0.025 mmol) and (3R)-pyrrolidin-3-ol (2.6 mg, 0.03 mmol) in 1 mL MeOH/DCM (1:1) was stirred at room temperature and acetic acid was added until the pH was around 4. After the reaction was stirred for 1 hour, sodium cyanoborohydride (7.9 mg, 0.13 mmol) was added and the reaction was stirred at room temperature for an additional 1 hour. Then, the reaction was quenched by addition of 10 mL MeOH and the solvent was removed under reduced pressure. The crude mixture was purified by column chromatography (silica gel, gradient elution, 10 to 60% MeOH/DCM). The fractions containing the product were collected and the solvent was removed under reduced pressure, resulting in the title compound. LCMS Method B, RT=3.067 min, m/z=709.0 [M+H]⁺, LCMS Method D, RT=3.568 min, m/z=709.2900 [M+H]⁺, exact mass: 708.2827 Example 134: methyl (4-(4-(2-chloro-3-(6-methoxy-5-(((((S)-5-oxopyrrolidin-2-yl)methyl)amino)methyl)pyridin-2-yl)phenyl)-1H-indazol-1-yl)-2,6-dimethoxybenzyl)-D-serinate

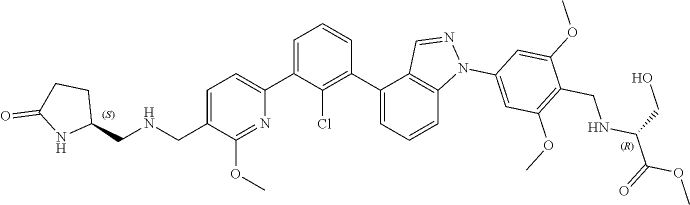

A mixture of Intermediate E3 (50 mg, 0.07 mmol) and D-Serine methyl ester hydrochloride (12.44 mg, 0.08 mmol) in 6 mL MeOH/CH₂Cl₂ (1:1) was stirred at room temperature and acetic acid was added until pH value around 4. After leaving for 1 hour, sodium cyanoborohydride (21.99 mg, 0.35 mmol) was added and the reaction was stirred at room temperature for an additional 1 hour before 4M HCl in dioxane (1 mL) was added. After 30 minutes, the solvent was removed under reduced pressure and the residue was washed with dioxane. The reaction mixture was purified by Sephadex column chromatography, affording the title compound. LCMS Method F, RT=6.469 min, m/z=729.3 [M+H]⁺, LCMS Method D, RT=3.578 min, m/z=729.2809 [M+H]⁺, exact mass: 728.2725. ¹H NMR (500 MHz, MeOD-d₄). δ 7.96 (s, 1H), 7.85 (d, J=8.6 Hz, 1H), 7.70 (d, J=7.4 Hz, 1H), 7.63 (dd, J=7.4, 1.9 Hz, 1H), 7.55 (dd, J=8.5, 8.5 Hz, 1H), 7.49 (dd, J=7.4, 7.4 Hz, 1H), 7.47 (dd, J=7.4, 1.9 Hz, 1H), 7.22 (d, J=7.4 Hz, 1H), 7.21 (d, J=7.0 Hz, 1H), 6.97 (s, 2H), 3.99 (s, 3H), 3.98-3.96 (m, 1H), 3.91 (d, J=4.2 Hz, 2H), 3.89 (s, 6H), 3.80 (d, J=6.2 Hz, 2H), 3.72-3.69 (m, 1H), 3.68-3.63 (m, 1H), 3.61 (s, 3H), 3.36 (t, J=5.6 Hz, 1H), 2.70-2.61 (m, 2), 2.31-2.21 (m, 3H), 1.81-1.73 (m, 1H).

### Example 135: (S)-2-((6-(2-fluoro-3-(1-(3,5-dimethoxy-4-((((5-oxopyrrolidin-2-yl)methyl)amino)methyl)phenyl)-1H-indazol-4-yl)phenyl)-2-methoxypyridin-3-yl)methyl)-2,6-diazaspiro[3.4]octan-7-one

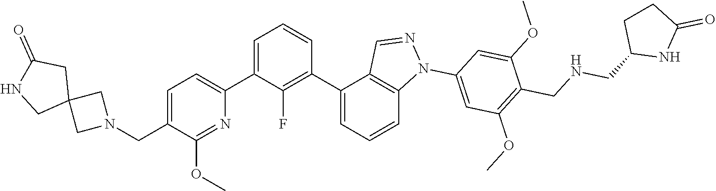 [SMILES: COc1cc(-n2ncc3c(-c4cccc(-c5ccc(CN6CC7(CNC(=O)C7)C6)c(OC)n5)c4F)cccc32)cc(OC)c1CNC[C@@H]1CCC(=O)N1]

### Step 1: 6-(3-bromo-2-fluorophenyl)-2-methoxynicotinaldehyde

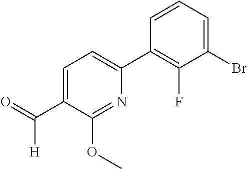 [SMILES: COc1nc(-c2cccc(Br)c2F)ccc1C=O]

This compound was prepared and purified by using similar procedures as described in the Intermediate C1 replacing 1,3-dibromo-2-chlorobenzene with 1,3-dibromo-2-fluorobenzene in order to obtain the desired product.

### Step 2: 2-((6-(3-bromo-2-fluorophenyl)-2-methoxypyridin-3-yl)methyl)-2,6-diazaspiro[3.4]octan-7-one

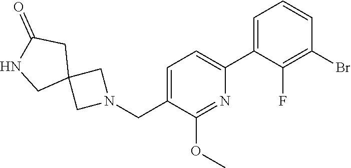 [SMILES: COc1nc(-c2cccc(Br)c2F)ccc1CN1CC2(CNC(=O)C2)C1]

A mixture of 6-(3-bromo-2-fluorophenyl)-2-methoxynicotinaldehyde (0.31 g, 1.0 mmol) and 2,6-diazaspiro[3.4]octan-7-one (0.19 g, 1.50 mmol) in 4 mL MeOH/DCM (1:1) was stirred at room temperature and acetic acid was added until the pH was around 4. After the reaction was stirred for 1 hour, sodium cyanoborohydride (0.30 g, 5.00 mmol) was added. The reaction was stirred at room temperature for an additional 2.5 hours, quenched by a small amount of MeOH and evaporated to dryness. The crude mixture was purified by Sephadex column chromatography (100% MeOH) to provide the product.

### Step 3: 4-(4-(2-fluoro-3-(6-methoxy-5-((7-oxo-2,6-diazaspiro[3.4]octan-2-yl)methyl)pyridin-2-yl)phenyl)-1H-indazol-1-yl)-2,6-dimethoxybenzaldehyde

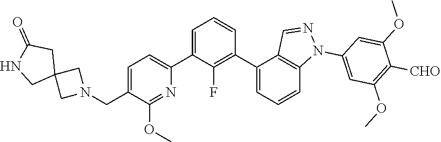 [SMILES: COc1cc(-n2ncc3c(-c4cccc(-c5ccc(CN6CC7(CNC(=O)C7)C6)c(OC)n5)c4F)cccc32)cc(OC)c1C=O]

A mixture of 2-((6-(3-bromo-2-fluorophenyl)-2-methoxypyridin-3-yl)methyl)-2,6-diazaspiro[3.4]octan-7-one (0.11 g, 0.26 mmol), Intermediate B4-1 (0.16 g, 0.39 mmol), Pd(PPh₃)₄ (32.0 mg, 0.026 mmol) and K₂CO₃ (0.13 g, 0.91 mmol) in a mixture of 1,4-dioxane and H₂O (6:1) 14 mL was sparged with argon for 10 min before stirring at 95° C. for 1 hour under argon atmosphere. Then, the mixture was cooled down and diluted with brine. The resulting mixture was extracted 3 times with EtOAc and the organic layer was collected, dried over anhydrous Na₂SO₄, filtered, and evaporated to dryness. The crude mixture was purified by column chromatography (silica gel, gradient elution, 20 to 80% EtOAc/hexanes) to provide the product.

### Step 4: (S)-2-((6-(2-fluoro-3-(1-(3,5-dimethoxy-4-((((5-oxopyrrolidin-2-yl)methyl)amino)methyl)phenyl)-1H-indazol-4-yl)phenyl)-2-methoxypyridin-3-yl)methyl)-2,6-diazaspiro[3.4]octan-7-one

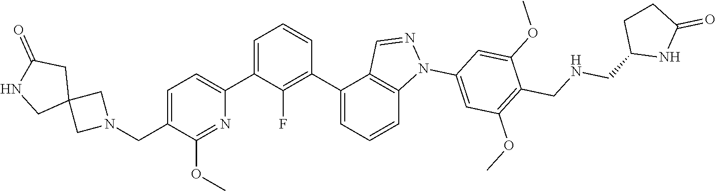 [SMILES: COc1cc(-n2ncc3c(-c4cccc(-c5ccc(CN6CC7(CNC(=O)C7)C6)c(OC)n5)c4F)cccc32)cc(OC)c1CNC[C@@H]1CCC(=O)N1]

A mixture of 4-(4-(2-fluoro-3-(6-methoxy-5-((7-oxo-2,6-diazaspiro[3.4]octan-2-yl)methyl)pyridin-2-yl)phenyl)-1H-indazol-1-yl)-2,6-dimethoxybenzaldehyde (30.0 mg, 0.040 mmol) and (S)-5-(aminomethyl)pyrrolidin-2-one (9.1 mg, 0.080 mmol) in 4 mL MeOH/DCM (1:1) was stirred at room temperature and added with acetic acid until the pH was around 4. After the reaction was stirred for 1 hour, sodium cyanoborohydride (12.6 mg, 0.20 mmol) was added and the reaction was stirred at room temperature for an additional 30 min. The crude mixture was purified by column chromatography (silica gel, gradient elution, 5 to 30% MeOH/DCM with addition of 0.5% TEA). The fractions containing the product were collected and the solvent was removed under reduced pressure, resulting the title compound. LCMS Method G, RT=6.901 min, m/z=720.4 [M+H]⁺, 743.3 [M+Na]⁺, LCMS Method D, RT=3.406 min, m/z=720.3318 [M+H]⁺, exact mass: 719.3231.

### Example 136: 2-(1-((6-(3-(1-(4-((3-(2-hydroxypropan-2-yl)azetidin-1-yl)methyl)-3,5-dimethoxyphenyl)-1H-indazol-4-yl)-2-methylphenyl)-2-methoxypyridin-3-yl)methyl)azetidin-3-yl)propan-2-ol

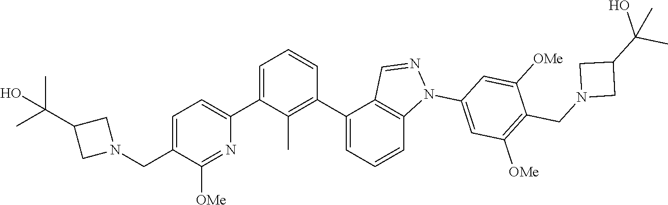 [SMILES: COc1cc(-n2ncc3c(-c4cccc(-c5ccc(CN6CC(C(C)(C)O)C6)c(OC)n5)c4C)cccc32)cc(OC)c1CN1CC(C(C)(C)O)C1]

### Step 1: 6-(3-bromo-2-methylphenyl)-2-methoxynicotinaldehyde

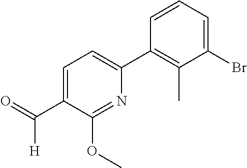 [SMILES: COc1nc(-c2cccc(Br)c2C)ccc1C=O]

This compound was prepared and purified by using similar procedures as described in the Intermediate C1 replacing 1,3-dibromo-2-chlorobenzene with 1,3-dibromo-2-methylbenzene to obtain the desired product.

### Step 2: 6-(3-(1-(4-formyl-3,5-dimethoxyphenyl)-1H-indazol-4-yl)-2-methylphenyl)-2-methoxynicotinaldehyde

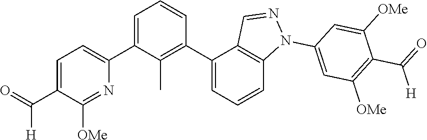 [SMILES: COc1cc(-n2ncc3c(-c4cccc(-c5ccc(C=O)c(OC)n5)c4C)cccc32)cc(OC)c1C=O]

A mixture of 6-(3-bromo-2-methylphenyl)-2-methoxynicotinaldehyde (0.12 g, 0.38 mmol), Intermediate B4-1 (0.29 g, 0.71 mmol), Pd(PPh₃)₄ (0.03 g, 0.03 mmol) and K₂CO₃ (0.13 g, 0.94 mmol) in a mixture of 1,4-dioxane and H₂O (6:1) 14 mL was sparged with argon for 10 min before stirring at 95° C. for 1 hour under argon atmosphere. Then, the mixture was cooled down and diluted with brine. The resulting mixture was extracted 3 times with EtOAc and the organic layer was collected, dried over anhydrous Na₂SO₄, filtered, and evaporated to dryness. The crude mixture was purified by column chromatography (silica gel, gradient elution, 20 to 80% EtOAc/hexanes) to provide the product.

### Step 3: 2-(1-((6-(3-(1-(4-((3-(2-hydroxypropan-2-yl)azetidin-1-yl)methyl)-3,5-dimethoxyphenyl)-1H-indazol-4-yl)-2-methylphenyl)-2-methoxypyridin-3-yl)methyl)azetidin-3-yl)propan-2-ol

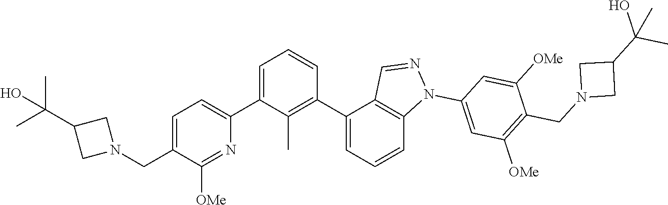 [SMILES: COc1cc(-n2ncc3c(-c4cccc(-c5ccc(CN6CC(C(C)(C)O)C6)c(OC)n5)c4C)cccc32)cc(OC)c1CN1CC(C(C)(C)O)C1]

A mixture of 6-(3-(1-(4-formyl-3,5-dimethoxyphenyl)-1H-indazol-4-yl)-2-methylphenyl)-2-methoxynicotinaldehyde (0.09 g, 0.17 mmol) and 1-(aminomethyl)cyclopropane-1-carboxamide (0.04 g, 0.37 mmol) in 4 mL MeOH/DCM (1:1) was stirred at room temperature and acetic acid was added until the pH was around 4. After the reaction was stirred for 1 hour, sodium cyanoborohydride (0.03 g, 0.51 mmol) was added and the reaction was stirred at room temperature for an additional 1 hour. All solvent was evaporated under vacuum and the crude mixture was purified by column chromatography (silica gel, gradient elution, 5 to 50% MeOH/DCM). The fractions containing the product were collected and the solvent was removed under reduced pressure, resulting in the title compound. LCMS Method G, RT=10.102 min, m/z=706.4 [M+H]⁺, LCMS Method D, RT=3.813 min, m/z=706.3964 [M+H]⁺, exact mass: 705.3890.

### Example 137: 2-((6-(2-chloro-3-(1-(4-((3-(2-hydroxypropan-2-yl)azetidin-1-yl)methyl)-3,5-dimethoxyphenyl)-1H-indazol-4-yl)phenyl)-2-methoxypyridin-3-yl)methyl)-2,6-diazaspiro[3.4]octan-7-one

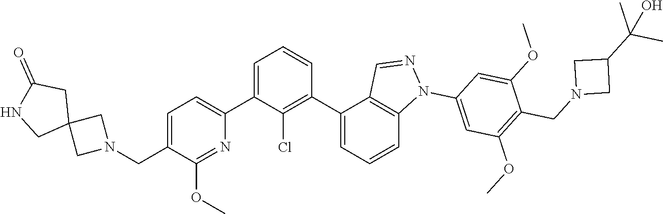 [SMILES: COc1cc(-n2ncc3c(-c4cccc(-c5ccc(CN6CC7(CNC(=O)C7)C6)c(OC)n5)c4Cl)cccc32)cc(OC)c1CN1CC(C(C)(C)O)C1]

A mixture of Intermediate E6 (20.0 mg, 0.031 mmol) and 2-(azetidine-3-yl)propan-2-ol trifluoroacetic acid (14.4 mg, 0.063 mmol) in 3 mL MeOH/DCM (1:1) was stirred at room temperature and added with acetic acid until the pH was around 4. After the reaction was stirred for 2 hours, sodium cyanoborohydride (9.85 mg, 0.157 mmol) was added and the reaction was stirred at room temperature for an additional 30 min. The crude mixture was purified by column chromatography (silica gel, gradient elution, 0 to 30% MeOH/DCM with addition of 0.5% TEA). The fractions containing the product were collected and the solvent was removed under reduced pressure, resulting in the title compound. LCMS Method G, RT=8.432 min, m/z=737.4 [M+H]⁺, LCMS Method D, RT=3.580 min, m/z=737.3219 [M+H]⁺, exact mass: 736.3140.

### Example 138: 2-(4-(4-(2-chloro-3-(5-((3-(2-hydroxypropan-2-yl)azetidin-1-yl)methyl)-6-methoxypyridin-2-yl)phenyl)-1H-indazol-1-yl)-2,6-dimethoxybenzyl)-2,6-diazaspiro[3.4]octan-7-one

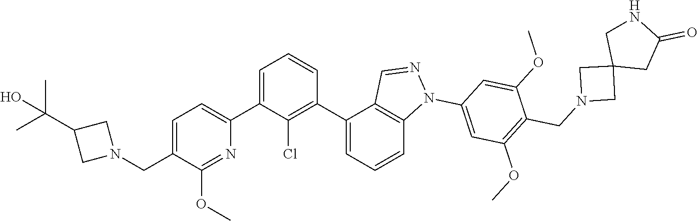 [SMILES: COc1cc(-n2ncc3c(-c4cccc(-c5ccc(CN6CC(C(C)(C)O)C6)c(OC)n5)c4Cl)cccc32)cc(OC)c1CN1CC2(CNC(=O)C2)C1]

The preparation of intermediate 6-(2-chloro-3-(1-(3,5-dimethoxy-4-((7-oxo-2,6-diazaspiro[3.4]octan-2-yl)methyl)phenyl)-1H-indazol-4-yl)phenyl)-2-methoxynicotinaldehyde was described under Example 130. A mixture of 6-(2-chloro-3-(1-(3,5-dimethoxy-4-((7-oxo-2,6-diazaspiro[3.4]octan-2-yl)methyl)phenyl)-1H-indazol-4-yl)phenyl)-2-methoxynicotinaldehyde (0.11 g, 0.17 mmol) and 1-(aminomethyl)cyclopropane-1-carboxamide (0.02 g, 0.20 mmol) in 4 mL MeOH/DCM (1:1) was stirred at room temperature and acetic acid was added until the pH was around 4. After the reaction was stirred for 1 hour, sodium cyanoborohydride (0.03 g, 0.51 mmol) was added and the reaction was stirred at room temperature for an additional 1 hour. All solvent was evaporated under vacuum and the crude mixture was purified by column chromatography (silica gel, gradient elution, 5 to 50% MeOH/DCM). The fractions containing the product were collected and the solvent was removed under reduced pressure, resulting the title compound. LCMS Method G, RT=8.418 min, m/z=737.3 [M+H]⁺, LCMS Method D, RT=3.563 min, m/z=737.3202 [M+H]⁺, exact mass: 736.3140.

### Example 139: (S)-2-((4-(4-(2-chloro-3-(6-methoxy-5-((7-oxo-2,6-diazaspiro[3.4]octan-2-yl)methyl)pyridin-2-yl)phenyl)-1H-indazol-1-yl)-2,6-dimethoxybenzyl)amino)-3-hydroxy-2-methylpropanoic acid

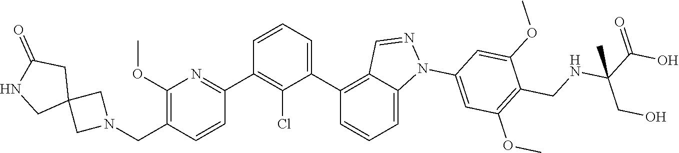 [SMILES: COc1cc(-n2ncc3c(-c4cccc(-c5ccc(CN6CC7(CNC(=O)C7)C6)c(OC)n5)c4Cl)cccc32)cc(OC)c1CN[C@@](C)(CO)C(=O)O]

A mixture of Intermediate E6 (50 mg, 0.08 mmol) and (2S)-2-amino-3-hydroxy-2-methylpropanoic acid (14.0 mg, 0.12 mmol) in 4 mL MeOH/DCM (1:1) was stirred at room temperature and acetic acid was added until the pH was around 4. After the reaction was stirred for 1 hour, sodium cyanoborohydride (25 mg, 0.51 mmol) was added and the reaction was stirred at room temperature for an additional 1 hour. The crude mixture was purified by column chromatography (silica gel, gradient elution, 5 to 50% MeOH/DCM). The fractions containing the product were collected and the solvent was removed under reduced pressure, resulting in the title compound. LCMS Method G, RT=7.506 min, m/z=763.3 [M+Na]⁺, LCMS Method D, RT=3.452 min, m/z=741.2738 [M+H]⁺, exact mass: 740.2725.

### Example 140: 2-(4-(4-(2-chloro-3-(5-((3-(2-hydroxypropan-2-yl)azetidin-1-yl)methyl)-6-methoxypyridin-2-yl)phenyl)-1H-indazol-1-yl)-2,6-dimethoxybenzyl)-2,5-diazaspiro[3.4]octan-6-one

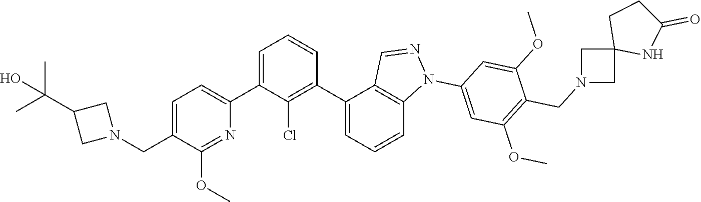 [SMILES: COc1cc(-n2ncc3c(-c4cccc(-c5ccc(CN6CC(C(C)(C)O)C6)c(OC)n5)c4Cl)cccc32)cc(OC)c1CN1CC2(CCC(=O)N2)C1]

### Step 1: 2-(4-(4-bromo-1H-indazol-1-yl)-2,6-dimethoxybenzyl)-2,5-diazaspiro[3.4]octan-6-one

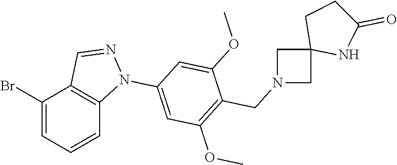 [SMILES: COc1cc(-n2ncc3c(Br)cccc32)cc(OC)c1CN1CC2(CCC(=O)N2)C1]

A mixture of Intermediate B4 (0.20 g, 0.55 mmol) and 2,5-diazaspiro[3.4]octan-6-one (0.08 g, 0.66 mmol) in 4 mL MeOH/DCM (1:1) was stirred at room temperature and acetic acid was added until the pH was around 4. After the reaction was stirred for 1 hour, sodium cyanoborohydride (0.15 g, 2.30 mmol) was added. The reaction was stirred at room temperature for an additional 2.5 hours, quenched by a small amount of MeOH and evaporated to dryness. The crude mixture was purified by Sephadex column chromatography (100% MeOH) to provide the product.

### Step 2: 6-(2-chloro-3-(1-(3,5-dimethoxy-4-((6-oxo-2,5-diazaspiro[3.4]octan-2-yl)methyl)phenyl)-1H-indazol-4-yl)phenyl)-2-methoxynicotinaldehyde

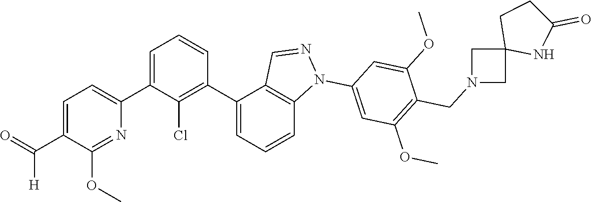 [SMILES: COc1cc(-n2ncc3c(-c4cccc(-c5ccc(C=O)c(OC)n5)c4Cl)cccc32)cc(OC)c1CN1CC2(CCC(=O)N2)C1]

A mixture of 2-(4-(4-bromo-1H-indazol-1-yl)-2,6-dimethoxybenzyl)-2,6-diazaspiro[3.4]octan-7-one (0.18 g, 0.38 mmol), Intermediate C2 (0.27 g, 0.71 mmol), Pd(PPh₃)₄ (0.03 g, 0.03 mmol) and K₂CO₃ (0.13 g, 0.94 mmol) in a mixture of 1,4-dioxane and H₂O (6:1) 14 mL was sparged with argon for 10 min before stirring at 95° C. for 1 hour under argon atmosphere. Then, the mixture was cooled down and diluted with brine. The resulting mixture was extracted 3 times with EtOAc and the organic layer was collected, dried over anhydrous Na₂SO₄, filtered, and evaporated to dryness. The crude mixture was purified by column chromatography (silica gel, gradient elution, 5 to 50% MeOH/DCM) to provide the product.

### Step 3: 2-(4-(4-(2-chloro-3-(5-((3-(2-hydroxypropan-2-yl)azetidin-1-yl)methyl)-6-methoxypyridin-2-yl)phenyl)-1H-indazol-1-yl)-2,6-dimethoxybenzyl)-2,5-diazaspiro[3.4]octan-6-one

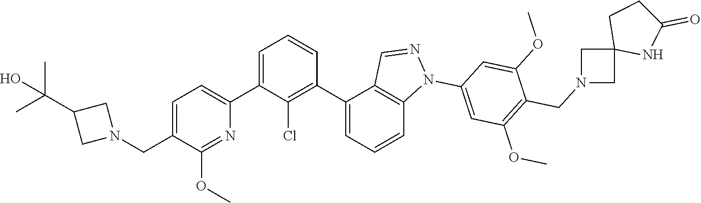 [SMILES: COc1cc(-n2ncc3c(-c4cccc(-c5ccc(CN6CC(C(C)(C)O)C6)c(OC)n5)c4Cl)cccc32)cc(OC)c1CN1CC2(CCC(=O)N2)C1]

A mixture of 6-(2-chloro-3-(1-(3,5-dimethoxy-4-((6-oxo-2,5-diazaspiro[3.4]octan-2-yl)methyl)phenyl)-1H-indazol-4-yl)phenyl)-2-methoxynicotinaldehyde (0.11 g, 0.17 mmol) and 1-(aminomethyl)cyclopropane-1-carboxamide (0.02 g, 0.20 mmol) in 4 mL MeOH/DCM (1:1) was stirred at room temperature and acetic acid was added until the pH was around 4. After the reaction was stirred for 1 hour, sodium cyanoborohydride (0.03 g, 0.51 mmol) was added and the reaction was stirred at room temperature for an additional 1 hour. All solvent was evaporated under vacuum and the crude mixture was purified by column chromatography (silica gel, gradient elution, 5 to 50% MeOH/DCM). The fractions containing the product were collected and the solvent was removed under reduced pressure, resulting the title compound. LCMS Method F, RT=6.806 min, m/z=737.3 [M+H]⁺, LCMS Method D, RT=3.563 min, m/z=737.3197 [M+H]⁺, exact mass: 736.3140.

### Example 141: (4-(4-(2-chloro-3-(6-methoxy-5-((7-oxo-2,6-diazaspiro[3.4]octan-2-yl)methyl)pyridin-2-yl)phenyl)-1H-indazol-1-yl)-2,6-dimethoxybenzyl)-L-threonine

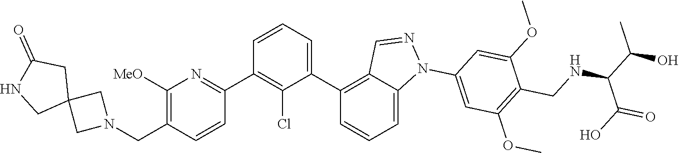 [SMILES: COc1cc(-n2ncc3c(-c4cccc(-c5ccc(CN6CC7(CNC(=O)C7)C6)c(OC)n5)c4Cl)cccc32)cc(OC)c1CN[C@H](C(=O)O)[C@@H](C)O]

A mixture of Intermediate E6 (60 mg, 0.094 mmol) and (2S,3R)-2-amino-3-hydroxybutanoic acid (13.4 mg, 0.113 mmol) in 4 mL MeOH/DCM (1:1) was stirred at room temperature and acetic acid was added until the pH was around 4. After the reaction was stirred for 1 hour, sodium cyanoborohydride (99.6 mg, 0.47 mmol) was added and the reaction was stirred at room temperature for an additional 1 hour. Then, the reaction was quenched by addition of 30 mL MeOH and the solvent was removed under reduced pressure. The crude mixture was purified by column chromatography (silica gel, gradient elution, 10 to 30% MeOH/DCM with addition of 1% TEA). The fractions containing the product were collected and the solvent was removed under reduced pressure, resulting in the title compound. LCMS Method G, RT=7.133 min, m/z=741.3 [M+H]⁺, LCMS Method D, RT=3.441 min, m/z=741.2769 [M+H]⁺, exact mass: 740.2725.

### Example 142: 2-(1-((6-(2-chloro-3-(1-(5-(((2-hydroxyethyl)amino)methyl)-4-methoxypyridin-2-yl)-1H-indazol-4-yl)phenyl)-2-methoxypyridin-3-yl)methyl)azetidin-3-yl)propan-2-ol

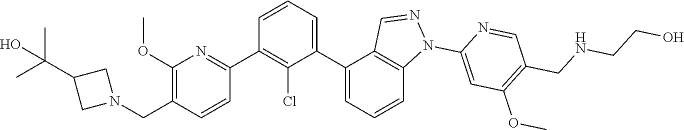 [SMILES: COc1cc(-n2ncc3c(-c4cccc(-c5ccc(CN6CC(C(C)(C)O)C6)c(OC)n5)c4Cl)cccc32)ncc1CNCCO]

### Step 1: 2-(((6-(4-bromo-1H-indazol-1-yl)-4-methoxypyridin-3-yl)methyl)amino)ethan-1-ol

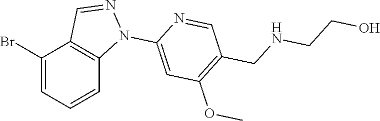 [SMILES: COc1cc(-n2ncc3c(Br)cccc32)ncc1CNCCO]

A mixture of Intermediate B5 (170 mg, 0.51 mmol) and 2-aminoethan-1-ol (36.4 μL, 0.61 mmol) in 15 mL MeOH/DCM (1:1) was stirred at room temperature and added with acetic acid until the pH was around 4. After the reaction was stirred for 1 hour, sodium cyanoborohydride (160 mg, 2.55 mmol) was added and the reaction was stirred at room temperature for an additional 1 hour. The crude mixture was used without purification.

### Step 2: tert-butyl ((6-(4-bromo-1H-indazol-1-yl)-4-methoxypyridin-3-yl)methyl)(2-hydroxyethyl)carbamate

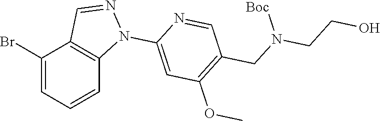 [SMILES: COc1cc(-n2ncc3c(Br)cccc32)ncc1CN(CCO)C(=O)OC(C)(C)C]

A mixture of 2-(((6-(4-bromo-1H-indazol-1-yl)-4-methoxypyridin-3-yl)methyl)amino)ethan-1-ol (190 mg, 0.50 mmol) was dissolved in MeOH and di-tert-butyldicarbonate (165 mg, 0.76 mmol), triethylamine (92 μL, 0.66 mmol), and 4-dimethylaminopyridine were added. After the reaction was stirred at room temperature for 2 h, water was added into the solution. The resulting mixture was extracted 3 times with EtOAc and the organic layer was collected, washed with brine, dried over anhydrous Na₂SO₄, filtered, and evaporated to dryness. The crude mixture was purified by column chromatography (silica gel, gradient elution, 30 to 50% EtOAc/hexanes).

### Step 3: tert-butyl ((6-(4-(2-chloro-3-(5-formyl-6-methoxypyridin-2-yl)phenyl)-1H-indazol-1-yl)-4-methoxypyridin-3-yl)methyl)(2-hydroxyethyl)carbamate

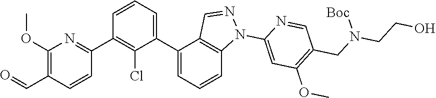 [SMILES: COc1cc(-n2ncc3c(-c4cccc(-c5ccc(C=O)c(OC)n5)c4Cl)cccc32)ncc1CN(CCO)C(=O)OC(C)(C)C]

A mixture of Intermediate C2 (235 mg, 0.63 mmol), tert-butyl ((6-(4-bromo-1H-indazol-1-yl)-4-methoxypyridin-3-yl)methyl)(2-hydroxyethyl)carbamate (230 mg, 0.48 mmol), Pd(PPh₃)₄ (56 mg, 0.05 mmol) and K₂CO₃ (160 mg, 1.16 mmol) in a mixture of 1,4-dioxane and H₂O (6:1) 30 mL was sparged with argon for 10 min before stirring at 95° C. for 3 h under argon atmosphere. Then, the mixture was cooled down and diluted with brine. The resulting mixture was extracted 3 times with EtOAc and the organic layer was collected, dried over anhydrous Na₂SO₄, filtered, and evaporated to dryness. The crude mixture was purified by column chromatography (silica gel, gradient elution, 50% EtOAc/hexanes).

### Step 4: 2-(1-((6-(2-chloro-3-(1-(5-(((2-hydroxyethyl)amino)methyl)-4-methoxypyridin-2-yl)-1H-indazol-4-yl)phenyl)-2-methoxypyridin-3-yl)methyl)azetidin-3-yl)propan-2-ol

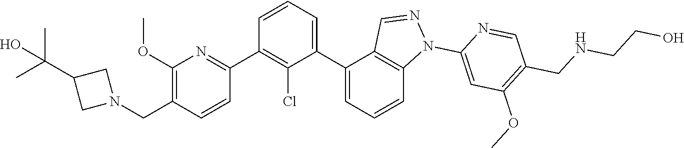 [SMILES: COc1cc(-n2ncc3c(-c4cccc(-c5ccc(CN6CC(C(C)(C)O)C6)c(OC)n5)c4Cl)cccc32)ncc1CNCCO]

A mixture of tert-butyl ((6-(4-(2-chloro-3-(5-formyl-6-methoxypyridin-2-yl)phenyl)-1H-indazol-1-yl)-4-methoxypyridin-3-yl)methyl)(2-hydroxyethyl)carbamate (81 mg, 0.13 mmol) and 2-(azetidin-3-yl)propan-2-ol (43 mg, 0.19 mmol) in 10 mL MeOH/DCM (1:1) was stirred at room temperature and added with acetic acid until the pH was around 4. After the reaction was stirred for 1 hour, sodium cyanoborohydride (40 mg, 0.63 mmol) was added and the reaction was stirred at room temperature for an additional 1 hour. The crude mixture was purified by column chromatography (silica gel, gradient elution, 0 to 10% MeOH/EtOAc). The fractions containing the product were collected and the solvent was removed under reduced pressure. The resulting oil was treated with 4M HCl in dioxane and stirred at room temperature for 30 min. After evaporation to dryness, the crude mixture was purified by column chromatography (silica gel, gradient elution, 0 to 30% MeOH/DCM). The fractions containing the product were collected and the solvent was removed under reduced pressure, resulting the title compound. LCMS Method G, RT=7.246 min, m/z=643.3 [M+H]⁺, LCMS Method D, RT=3.423 min, m/z=643.2778 [M+H]⁺, exact mass: 642.2721.

### Example 143: (2S,3R)-methyl 2-((4-(4-(2-chloro-3-(6-methoxy-5-(((((S)-5-oxopyrrolidin-2-yl)methyl)amino)methyl)pyridin-2-yl)phenyl)-1H-indazol-1-yl)-2,6-dimethoxybenzyl)amino)-3-hydroxybutanoate

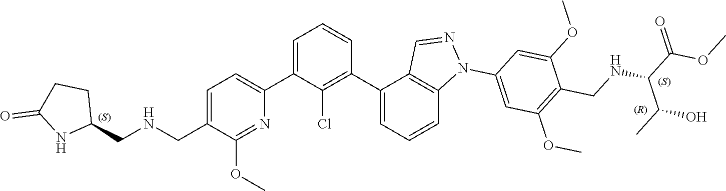

A mixture of Intermediate E3 (50 mg, 0.07 mmol) and L-threonine methyl ester (10.65 mg, 0.08 mmol) in 6 mL MeOH/CH₂Cl₂ (1:1) was stirred at room temperature and acetic acid was added until getting pH value around 4. After leaving for 1 hour, sodium cyanoborohydride (21.99 mg, 0.35 mmol) was added and the reaction was stirred at room temperature for an additional 1 hour. The residue was added with 4M HCl in dioxane (1 mL) and stirred at room temperature for 30 minutes. The residual solvent was removed under reduced pressure and washed with dioxane until pH value approximated to 5-6. The crude mixture was purified by Sephadex column chromatography, affording the title compound. LCMS Method G, RT=7.627 min, m/z=743.3 [M+H]⁺, LCMS Method D, RT=3.502 min, m/z=743.2925 [M+H]⁺, exact mass: 742.2882.

### Example 144: (2S,3R)-2-((4-(4-(2-chloro-3-(6-methoxy-5-(((((S)-5-oxopyrrolidin-2-yl)methyl)amino)methyl)pyridin-2-yl)phenyl)-1H-indazol-1-yl)-2,6-dimethoxybenzyl)amino)-3-hydroxybutanoic acid

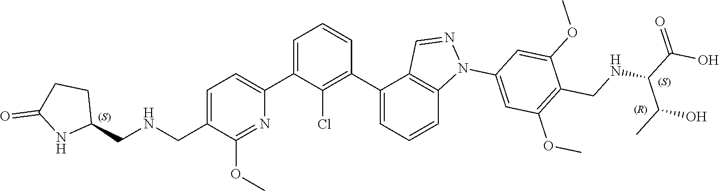

A mixture of Intermediate E3 (50 mg, 0.07 mmol) and L-threonine (9.53 mg, 0.08 mmol) in 6 mL 1,4-dioxane/H₂O (1:1) was stirred at room temperature and acetic acid was added until getting pH value around 4. After leaving for 1 hour, sodium cyanoborohydride (21.99 mg, 0.35 mmol) was added and the reaction was stirred at room temperature for an additional 1 hour before 4M HCl in dioxane (1 mL) was added. After 30 minutes, the solvent was removed under reduced pressure and the residue was washed with dioxane. The reaction mixture was purified by Sephadex column chromatography, affording the title compound. LCMS Method G, RT=7.128 min, m/z=729.3 [M+H]⁺, LCMS Method D, RT=3.436 min, m/z=729.2784 [M+H]⁺, exact mass: 728.2725. ¹H NMR (500 MHz, MeOD-d₄). δ 8.00 (s, 2H), 7.93 (d, J=8.6 Hz, 1H), 7.78 (d, J=7.4 Hz, 1H), 7.64 (dd, J=7.4, 1.9 Hz, 1H), 7.60 (dd, J=8.4, 8.3 Hz, 1H), 7.52 (dd, 0.1=7.5, 7.4 Hz, 1H), 7.49 (dd, J=7.6, 2.0 Hz, 1H), 7.28 (d, J=7.4 Hz, 1H), 7.26 (d, J=7.1 Hz, 1H), 7.10 (s, 2H), 4.39 (s, 2H), 4.03 (s, 3H), 3.98 (s, 8H), 3.95-3.87 (m, 1H), 3.37-3.31 (m, 1H), 3.20 (d, J=6.9 Hz, 1H), 3.16-2.98 (m, 2H), 2.91-2.80 (m, 1H), 2.41-2.28 (m, 2H), 1.91-1.75 (m, 1H), 1.34 (d, J=6.2 Hz, 3H).

### Example 145: Ethyl (4-(4-(2-chloro-3-(6-methoxy-5-((7-oxo-2,6-diazaspiro[3.4]octan-2-yl)methyl)pyridin-2-yl)phenyl)-1H-indazol-1-yl)-2,6-dimethoxybenzyl)-L-threoninate

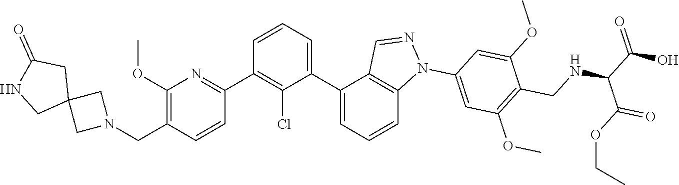

### Step 1: ethyl L-threoninate

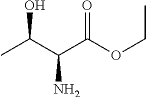

Ethanol (50 mL) was cooled on ice and SOCl₂ (3.6 mL, 50 mmol) was added dropwise. After the addition was complete, L-threoninate (1.05 g, 10 mmol) was added as a single portion and the reaction mixture was stirred 1 h at 0° C. followed by 16 h at room temperature. Then, the reaction mixture was concentrated to an oil, which was triturated with ice-cold diethyl ether to give ethyl ester.

### Step 2: Ethyl (4-(4-(2-chloro-3-(6-methoxy-5-((7-oxo-2,6-diazaspiro[3.4]octan-2-yl)methyl)pyridin-2-yl)phenyl)-1H-indazol-1-yl)-2,6-dimethoxybenzyl)-L-threoninate

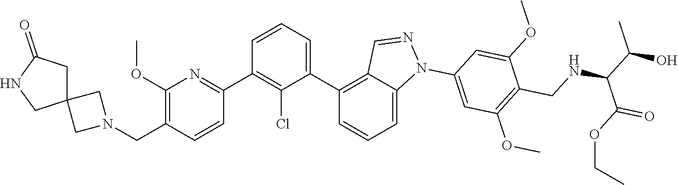 [SMILES: CCOC(=O)[C@@H](NCc1c(OC)cc(-n2ncc3c(-c4cccc(-c5ccc(CN6CC7(CNC(=O)C7)C6)c(OC)n5)c4Cl)cccc32)cc1OC)[C@@H](C)O]

A mixture of Intermediate E6 (50 mg, 0.08 mmol) and ethyl L-threoninate (28.8 mg, 0.2 mmol) in 4 mL MeOH/DCM (1:1) was stirred at room temperature and acetic acid was added until the pH was around 4. After the reaction was stirred for 1 hour, sodium cyanoborohydride (25 mg, 0.51 mmol) was added and the reaction was stirred at room temperature for an additional 1 hour. The crude mixture was purified by column chromatography (silica gel, gradient elution, 5 to 50% MeOH/DCM). The fractions containing the product were collected and the solvent was removed under reduced pressure, resulting in the title compound. LCMS Method G, RT=8.746 min, m/z=769.3 [M+H]⁺, LCMS Method D, RT=3.601 min, m/z=769.3109 [M+H]⁺, exact mass. 768.3038.

### Example 146: (2S,3R)-ethyl 2-((4-(4-(2-chloro-3-(6-methoxy-5-(((((S)-5-oxopyrrolidin-2-yl)methyl)amino)methyl)pyridin-2-yl)phenyl)-1H-indazol-1-yl)-2,6-dimethoxybenzyl)amino)-3-hydroxybutanoate

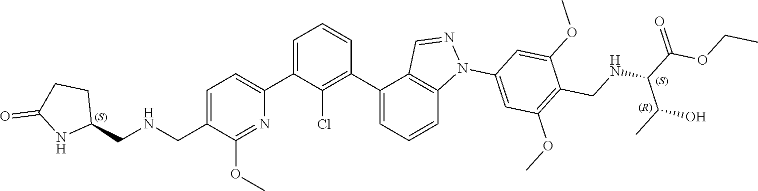

A mixture of Intermediate E3 (50 mg, 0.07 mmol) and L-threonine ethyl ester (11.77 mg, 0.08 mmol) in 6 mL MeOH/CH₂Cl₂ (1:1) was stirred at room temperature and acetic acid was added until getting pH value around 4. After leaving for 1 hour, sodium cyanoborohydride (21.99 mg, 0.35 mmol) was added and the reaction was stirred at room temperature for an additional 1 hour. The residue was added with 4M HCl in dioxane (1 mL) and stirred at room temperature for 30 minutes. The residual solvent was removed under reduced pressure and washed with dioxane until pH value approximated to 5-6. The crude mixture was purified by Sephadex column chromatography, affording the title compound. LCMS Method G, RT=8.631 min, m/z=757.3 [M+H]⁺, LCMS Method D, RT=3.601 min, m/z=757.3102 [M+H]⁺, exact mass: 756.3038.

Example 147: Ethyl (2R)-2-[({4-[4-(2-chloro-3-{6-methoxy-5-[({[(2S)-5-oxopyrrolidin-2-yl]methyl)amino)methyl}pyridin-2-yl}phenyl)-1H-indazol-1-yl]-2,6-dimethoxyphenyl}methyl)amino]-3-hydroxypropanoate

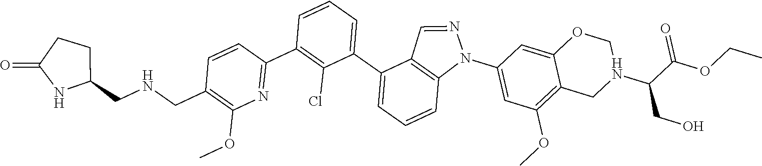

A mixture of Intermediate E3 (42 mg, 0.058 mmol) and ethyl (2R)-2-amino-3-hydroxypropanoate hydrochloride (14.7 mg, 0.09 mmol) in 2 mL MeOH/DCM (1:1) was stirred at room temperature and acetic acid was added until the pH was around 4. After the reaction was stirred for 1 hour, sodium cyanoborohydride (18.2 mg, 0.29 mmol) was added and the reaction was stirred at room temperature for an additional 1 hour. Then, the reaction was quenched by addition of 10 mL MeOH and the solvent was removed under reduced pressure. Then, the residue was dissolved in 1 mL DCM/trifluoroacetic acid (1:1). After stirring at room temperature for 30 minutes, the residual solvent was removed under reduced pressure. The crude mixture was purified by column chromatography (silica gel, gradient elution, 2 to 10% MeOH/DCM with addition of 1% TEA). The fractions containing the product were collected and the solvent was removed under reduced pressure, resulting in the title compound. LCMS Method G, RT=8.185 min, m/z=765.3 [M+Na]⁺, LCMS Method D, RT=3.539 min, m/z=743.2861 [M+H]⁺, exact mass: 742.2882.

### Example 148: (S)-1-(4-(4-(2-chloro-3-(6-methoxy-5-((((5-oxopyrrolidin-2-yl)methyl)amino)methyl)pyridin-2-yl)phenyl)-1H-indazol-1-yl)-2,6-dimethoxybenzyl)azetidine-3-carboxylic acid

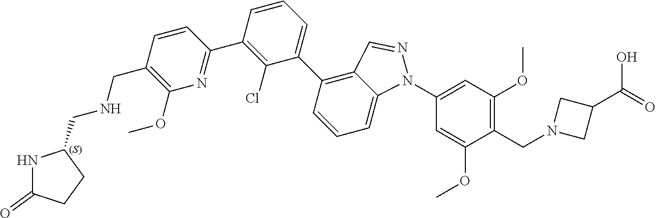

A mixture of Intermediate E3 (50.00 mg, 0.06 mmol) and azetidine-3-carboxylic acid (11.04 mg, 0.096 mmol) in 4 mL MeOH/DCM (1:1) was stirred at room temperature and added with acetic acid until the pH was around 4. After the reaction was stirred for 1 hour, sodium cyanoborohydride (15.12 mg, 0.24 mmol). The reaction was stirred at room temperature for an additional 1 hour, quenched by a small amount of MeOH and evaporated to dryness. Then, the residue was dissolved in DCM and trifluoroacetic acid (0.2 mL) was added. After stirring at room temperature for 30 minutes, the residual solvent was removed under reduced pressure. The crude mixture was purified by column chromatography (silica gel, gradient elution, 5 to 50% MeOH/DCM with addition of 0.5% TEA). The fractions containing the product were collected and the solvent was removed under reduced pressure, resulting the title compound. LCMS method G, RT=7.334 min, m/z=711.3 [M+H]⁺, LCMS Method D, RT=3.413 min, m/z=711.2603 [M+H]⁺, exact mass: 710.2620. ¹H NMR (500 MHz, MeOD-d₄) δ 8.02 (s, 1H), 7.94 (d, J=8.6 Hz, 1H), 7.75 (d, J=7.5 Hz, 1H), 7.67-7.59 (m, 2H), 7.55-7.48 (m, 2H), 7.27 (dd, J=7.2, 3.5 Hz, 2H), 7.13 (s, 2H), 4.46 (s, 2H), 4.27-4.19 (m, 4H), 4.02 (s, 3H), 3.99 (s, 6H), 3.92-3.83 (m, 2H), 3.44 (dt, J=8.9, 5.7 Hz, 2H), 3.32 (dt, J=15.3, 4.9 Hz, 2H), 2.80-2.70 (m, 1H), 2.35-2.23 (m, 2H), 1.85-1.76 (m, 1H).

### Example 149: (S)-methyl 1-(4-(4-(2-chloro-3-(6-methoxy-5-((((5-oxopyrrolidin-2-yl)methyl)amino)methyl)pyridin-2-yl)phenyl)-1H-indazol-1-yl)-2,6-dimethoxybenzyl)azetidine-3-carboxylate

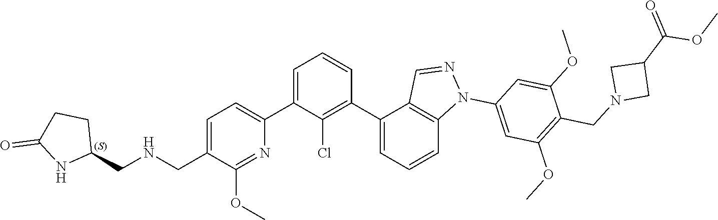

A mixture of Intermediate E3 (50 mg, 0.07 mmol) and methyl azetidine-3-carboxylate hydrochloride (12.13 mg, 0.08 mmol) in 6 mL MeOH/CH₂Cl₂ (1:1) was stirred at room temperature and acetic acid was added until pH value around 4. After leaving for 1 hour, sodium cyanoborohydride (21.99 mg, 0.35 mmol) was added and the reaction was stirred at room temperature for an additional 1 hour before 4M HCl in dioxane (1 mL) was added. After 30 minutes, the solvent was removed under reduced pressure and the residue was washed with dioxane. The reaction mixture was purified by Sephadex column chromatography (100% MeOH), affording the title compound. LCMS Method G, RT=8.444 min, m/z=725.3 [M+H]⁺, exact mass: 724.2776. ¹H NMR (500 MHz, MeOD-d4). δ 8.03 (s, 1H), 7.94 (d, J=8.5 Hz, 1H), 7.84 (d, J=7.5 Hz, 1H), 7.67 (dd, J=7.4, 2.0 Hz, 1H), 7.63 (dd, J=8.5, 7.2 Hz, 1H), 7.55 (dd, J=7.5, 7.5 Hz, 1H), 7.52 (dd, J=7.6, 2.0 Hz, 1H), 7.34 (d, J=7.5 Hz, 1H), 7.28 (d, J=7.1 Hz, 1H), 7.14 (s, 2H), 4.50 (s, 2H), 4.38-4.28 (m, 3H), 4.20-4.11 (m, 2H), 4.06 (s, 3H), 4.00 (s, 6H), 4.02-3.93 (m, 2H), 3.77 (s, 3H), 3.76-3.67 (m, 1H), 3.20-3.16 (m, 2H), 3.08-3.02 (m, 1H), 2.38-2.31 (m, 2H), 1.90-1.82 (m, 1H).

### Example 150: 1-(4-(4-(2-chloro-3-(6-methoxy-5-((7-oxo-2,6-diazaspiro[3.4]octan-2-yl)methyl)pyridin-2-yl)phenyl)-1H-indazol-1-yl)-2,6-dimethoxybenzyl)azetidine-3-carboxylic acid

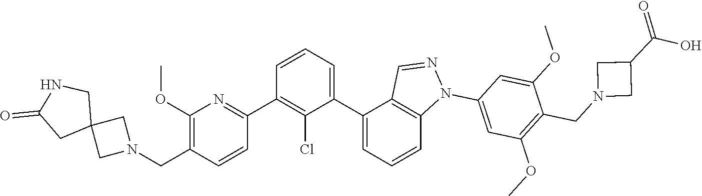 [SMILES: COc1cc(-n2ncc3c(-c4cccc(-c5ccc(CN6CC7(CNC(=O)C7)C6)c(OC)n5)c4Cl)cccc32)cc(OC)c1CN1CC(C(=O)O)C1]

A mixture of Intermediate E6 (62.0 mg, 0.10 mmol) and azetidine-3-carboxylic acid (24.7 mg, 0.24 mmol) in 5 mL MeOH/DCM (1:1) was stirred at room temperature and added with acetic acid until the pH was around 4. After the reaction was stirred for 1.5 hours, sodium cyanoborohydride (30.7 mg, 0.49 mmol) was added and the reaction was stirred at room temperature for an additional 30 min. The crude mixture was purified by column chromatography (silica gel, gradient elution, 0 to 30% MeOH/DCM). The fractions containing the product were collected and the solvent was removed under reduced pressure, resulting in the title compound. LCMS Method G, RT=7.293 min, m/z=723.3 [M+H]⁺, 745.2 [M+Na]⁺, exact mass: 722.2620.

### Example 151: ethyl (S)-1-(4-(4-(2-chloro-3-(6-methoxy-5-((((5-oxopyrrolidin-2-yl)methyl)amino)methyl)pyridin-2-yl)phenyl)-1H-indazol-1-yl)-2,6-dimethoxybenzyl)-3-methylazetidine-3-carboxylate

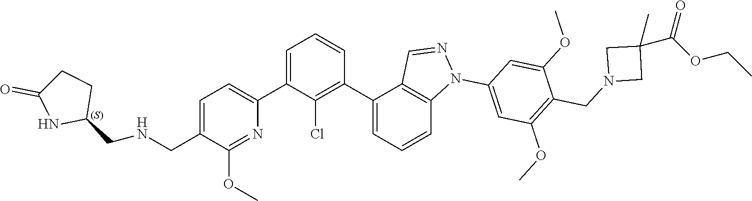

A mixture of Intermediate E3 (30.00 mg, 0.041 mmol) and ethyl 3-methylazetidine-3-carboxylate hydrochloride (14.7 mg, 0.082 mmol) in 5 mL MeOH/DCM (1.1) was stirred at room temperature and added with acetic acid until the pH was around 4. After the reaction was stirred for 1 hour, sodium cyanoborohydride (13.0 mg, 0.21 mmol). The reaction was stirred at room temperature for an additional 1 hour, quenched by a small amount of MeOH and evaporated to dryness. Then, the residue was dissolved in DCM and trifluoroacetic acid (0.3 mL) was added. After stirring at room temperature for 30 minutes, the residual solvent was removed under reduced pressure. The crude mixture was purified by Sephadex column chromatography (100% MeOH), affording the title compound. LCMS method G, RT=9.970 min, m/z=753.3 [M+H]⁺, exact mass: 752.3089.

### Example 152: 1-(4-(4-(2-chloro-3-(5-((((cis)-3-hydroxycyclobutyl)amino)methyl)-6-methoxypyridin-2-yl)phenyl)-1H-indazol-1-yl)-2,6-dimethoxybenzyl)azetidine-3-carboxylic acid

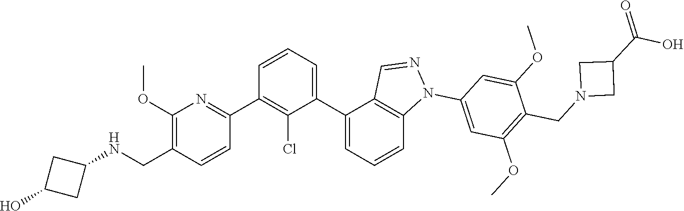 [SMILES: COc1cc(-n2ncc3c(-c4cccc(-c5ccc(CN[C@H]6C[C@@H](O)C6)c(OC)n5)c4Cl)cccc32)cc(OC)c1CN1CC(C(=O)O)C1]

A mixture of Intermediate E7 (130 mg, 0.19 mmol) and azetidine-3-carboxylic acid hydrochloride (47 mg, 0.47 mmol) in 6 mL MeOH/DCM (1:1) was stirred at room temperature and acetic acid was added until the pH was around 4. After the reaction was stirred for 1 hour, sodium cyanoborohydride (58 mg, 0.93 mmol) was added and the reaction was stirred at room temperature for an additional 1 hour. Then, the reaction was quenched by addition of 5 mL MeOH and the solvent was removed under reduced pressure. Then, the residue was dissolved in 1 mL DCM/trifluoroacetic acid (1:1). After stirring at room temperature for 30 minutes, the residual solvent was removed under reduced pressure. The crude mixture was purified by column chromatography (silica gel, gradient elution, 5 to 30% MeOH/DCM with addition of 0.3% TEA). The fractions containing the product were collected and the solvent was removed under reduced pressure, resulting in the title compound. LCMS Method G, RT=6.909 min, m/z=684.3 [M+H]⁺, exact mass: 683.2511. ¹H NMR (500 MHz, MeOD-d4) δ ppm 8.03 (s, 1H), 7.93 (d, J=8.5 Hz, 1H), 7.71 (d, J=7.5 Hz, 1H), 7.66 (dd, J=7.3, 2.0 Hz, 1H), 7.62 (d, J=15.7 Hz, 1H), 7.57-7.50 (m, 2H), 7.27 (dd, J=14.2, 7.2 Hz, 2H), 7.08 (s, 2H), 4.11 (s, 2H), 4.04 (s, 3H), 4.02-4.00 (m, 1H), 3.97 (s, 6H), 3.89-3.85 (m, 4H), 3.73 (s, 2H), 2.86-2.79 (m, 1H), 2.60-2.54 (m, 2H), 1.67 (m, 2H), 0.93-0.84 (m, 1H).

### Example 153: Methyl 1-(4-(4-(2-chloro-3-(5-((((cis)-3-hydroxycyclobutyl)amino)methyl)-6-methoxypyridin-2-yl)phenyl)-1H-indazol-1-yl)-2,6-dimethoxybenzyl)azetidine-3-carboxylate

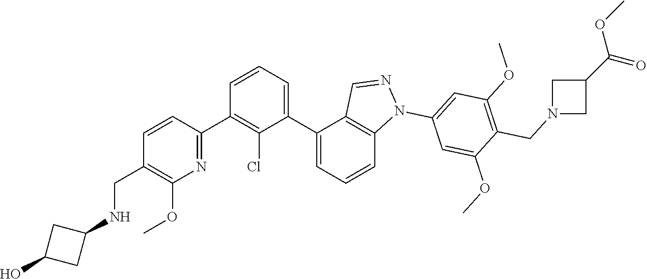 [SMILES: COC(=O)C1CN(Cc2c(OC)cc(-n3ncc4c(-c5cccc(-c6ccc(CN[C@H]7C[C@@H](O)C7)c(OC)n6)c5Cl)cccc43)cc2OC)C1]

Example 152 (15 mg) was dissolved in MeOH (excess) and then, 4M HCl in dioxane (excess) was added. After stirring at room temperature for 45 minutes, the residual solvent was removed under reduced pressure. The crude mixture was purified by column chromatography (silica gel, 10% MeOH/DCM). The fractions containing the product were collected and the solvent was removed under reduced pressure, resulting in the title compound. LCMS Method G, RT=8.460 min, m/z=698.3 [M+H]⁺, exact mass: 697.2667.

### Example 154: (R)-1-((7-cyano-2-(3-(1-(4-(((R)-3-hydroxypyrrolidin-1-yl)methyl)-3,5-dimethoxyphenyl)-1H-indazol-4-yl)-2-methylphenyl)benzo[d]oxazol-5-yl)methyl)pyrrolidine-3-carboxylic acid

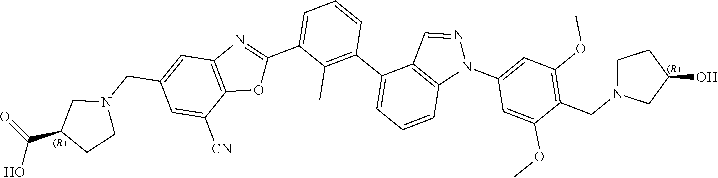

### Step 1: (R)-5-(hydroxymethyl)-2-(3-(1-(4-((3-hydroxypyrrolidin-1-yl)methyl)-3,5-dimethoxyphenyl)-1H-indazol-4-yl)-2-methylphenyl)benzo[d]oxazole-7-carbonitrile

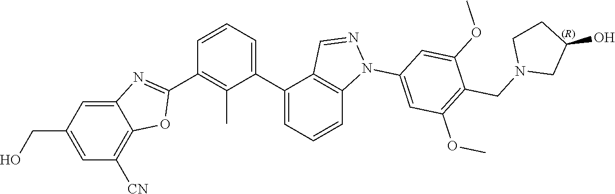

NEt₃ was added to a solution of (R)-pyrrolidin-3-ol hydrochloride (14.8 mg, 0.12 mmol) in 10 mL MeOH/DCM (1:1) until the pH was around 7. Then, the mixture was transferred to a mixture of Intermediate H (65.4 mg, 0.12 mmol) in 5 mL MeOH/DCM (1:1), acidified with acetic acid (pH around 4) and stirred for 1 hour. Sodium triacetoxyborohydride (126.6 mg, 0.60 mmol) was added and the reaction was stirred for an additional 2.5 hours. The complete reaction was quenched by a small amount of MeOH and evaporated to dryness. The crude mixture was purified by column chromatography (silica gel, gradient elution, 0 to 100% MeOH/DCM) to provide the title compound.

### Step 2: (R)-5-formyl-2-(3-(1-(4-((3-hydroxypyrrolidin-1-yl)methyl)-3,5-dimethoxyphenyl)-1H-indazol-4-yl)-2-methylphenyl)benzo[d]oxazole-7-carbonitrile

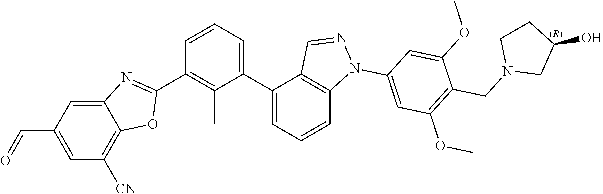

A solution of Dess-Martin periodinane (50.9 mg, 0.12 mmol) and pyridine (6 to 7 drops) in DCM 5 mL was added to a solution of (R)-5-(hydroxymethyl)-2-(3-(1-(4-((3-hydroxypyrrolidin-1-yl)methyl)-3,5-dimethoxyphenyl)-1H-indazol-4-yl)-2-methylphenyl)benzo[d]oxazole-7-carbonitrile (73.9 mg, 0.12 mmol) in DCM 5 mL at rt. The reaction was stirred for 30 minutes, evaporated to dryness, and purified by column chromatography (silica gel, gradient elution, 0 to 50% MeOH/DCM) to provide (R)-5-formyl-2-(3-(1-(4-((3-hydroxypyrrolidin-1-yl)methyl)-3,5-dimethoxyphenyl)-1H-indazol-4-yl)-2-methylphenyl)benzo[d]oxazole-7-carbonitrile.

### Step 3: (R)-1-((7-cyano-2-(3-(1-(4-(((R)-3-hydroxypyrrolidin-1-yl)methyl)-3,5-dimethoxyphenyl)-1H-indazol-4-yl)-2-methylphenyl)benzo[d]oxazol-5-yl)methyl)pyrrolidine-3-carboxylic acid

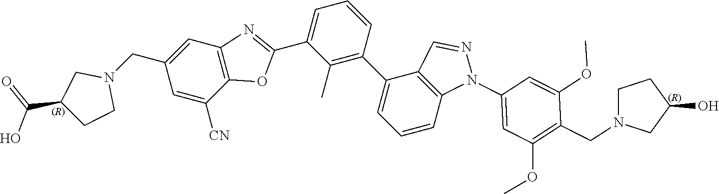

A mixture of (R)-5-formyl-2-(3-(1-(4-((3-hydroxypyrrolidin-1-yl)methyl)-3,5-dimethoxyphenyl)-1H-indazol-4-yl)-2-methylphenyl)benzo[d]oxazole-7-carbonitrile (73.6 mg, 0.12 mmol) and (R)-pyrrolidine-3-carboxylic acid (13.8 mg, 0.12 mmol) in 10 mL MeOH/DCM (1:1) was stirred at room temperature and acetic acid was added until pH value around 4. After leaving for 1 hour, sodium triacetoxyborohydride (251.0 mg, 1.18 mmol) was added and the reaction was stirred at room temperature for an additional 2.5 hour. The reaction was quenched by a small amount of MeOH and evaporated to dryness. The crude mixture was purified by column chromatography (silica gel, gradient elution, 0 to 100% MeOH/DCM) to provide the title compound. LCMS Method G, RT=9.133 min, m/z=713.3 [M+H]⁺, LCMS Method D, RT=3.667 min, m/z=713.3058 [M+H]⁺, exact mass: 712.3009. ¹H NMR (500 MHz, MeOD-d₄). δ 8.28 (dd, J=6.9, 2.4 Hz, 1H), 8.08 (d, J=1.5 Hz, 1H), 7.93 (d, J=0.9 Hz, 1H), 7.91 (d, J=8.6 Hz, 1H), 7.82 (d, J=1.5 Hz, 1H), 7.63 (dd, J=8.6, 7.0 Hz, 1H), 7.59-7.55 (m, 2H), 7.22 (dd, J=7.0, 0.5 Hz, 1H), 7.07 (s, 2H), 4.39-4.35 (m, 1H), 4.03 (s, 2H), 3.95 (s, 6H), 3.82 (d, J=8.0 Hz, 2H), 3.02-2.89 (m, 5H), 2.79-2.74 (m, 2H), 2.69-2.66 (m, 1H), 2.61-2.53 (m, 4H), 2.24-2.13 (m, 2H), 2.13-2.00 (m, 2H).

### Example 155: (R)-1-((7-cyano-2-(3-(1-(4-(((S)-3-hydroxypyrrolidin-1-yl)methyl)-3,5-dimethoxyphenyl)-1H-indazol-4-yl)-2-methylphenyl)benzo[d]oxazol-5-yl)methyl)pyrrolidine-3-carboxylic acid

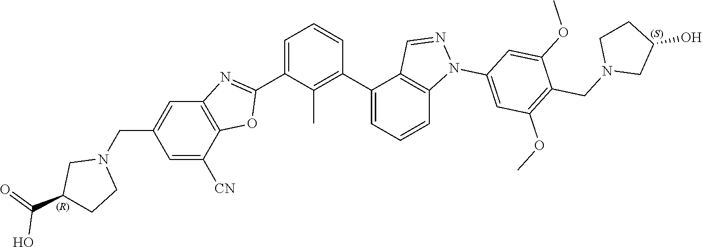

This compound was prepared using similar procedures as described for Example 154. The alcohol (R)-pyrrolidin-3-ol hydrochloride was replaced with (S)-pyrrolidin-3-ol hydrochloride in the Step 1. The crude mixture was purified by column chromatography (silica gel, gradient elution, 0 to 100% MeOH/DCM) to provide the title compound. LCMS Method G, RT=9.021 min, m/z=713.3 [M+H]⁺, LCMS Method D, RT=3.674 min, m/z=713.3066 [M+H]⁺, exact mass: 712.3009. ¹H NMR (500 MHz, MeOD-d₄). δ 8.27 (dd, J=7.0, 2.1 Hz, 1H), 8.08 (s, 1H), 7.92 (s, 1H), 7.90 (d, J=8.6 Hz, 1H), 7.82 (s, 1H), 7.62 (dd, J=8.5, 7.2 Hz, 1H), 7.60-7.54 (m, 2H), 7.21 (d, J=7.0 Hz, 1H), 7.03 (s, 2H), 4.33-4.27 (m, 1H), 3.92 (s, 6H), 3.86 (s, 2H), 3.80 (d, J=7.6 Hz, 2H), 3.00-2.88 (m, 3H), 2.82-2.73 (m, 2H), 2.69-2.63 (m, 2H), 2.59-2.52 (m, 5H), 2.09-1.99 (m, 4H).

Example 156: 4-(((6-(2-chloro-3-(1-(4-((3-hydroxy-3-methylazetidin-1-yl)methyl)-3,5-dimethoxyphenyl)-1H-indazol-4-yl)phenyl)-2-methoxypyridin-3-yl)methyl)(methyl)amino) bicyclo[2.2.2]octane-1-carboxylic acid

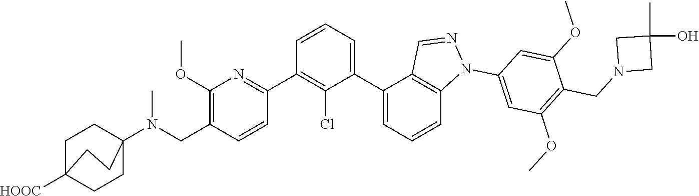 [SMILES: COc1cc(-n2ncc3c(-c4cccc(-c5ccc(CN(C)C67CCC(C(=O)O)(CC6)CC7)c(OC)n5)c4Cl)cccc32)cc(OC)c1CN1CC(C)(O)C1]

### Step 1: 1-(4-(4-bromo-1H-indazol-1-yl)-2,6-dimethoxybenzyl)-3-methylazetidin-3-ol

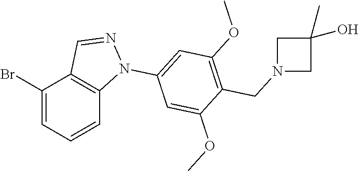 [SMILES: COc1cc(-n2ncc3c(Br)cccc32)cc(OC)c1CN1CC(C)(O)C1]

NEt₃ was added to a mixture of 3-methylazetidin-3-ol hydrochloride (137.0 mg, 1.11 mmol) in 5 mL MeOH/DCM (1:1) until the pH was around 7. Then, the mixture was transferred to a mixture of Intermediate B4 (0.200 g, 0.55 mmol) in 5 mL MeOH/DCM (1:1) at room temperature. After that, acetic acid was added until the pH was around 4. After the reaction was stirred for 1 hour, sodium cyanoborohydride (104.0 mg, 1.66 mmol) was added. The reaction was quenched by a small amount of MeOH and evaporated to dryness. The crude mixture was purified by column chromatography (silica gel, gradient elution, 0 to 10% MeOH/DCM) to provide the desired product.

### Step 2: 6-(2-chloro-3-(1-(4-((3-hydroxy-3-methylazetidin-1-yl)methyl)-3,5-dimethoxyphenyl)-1H-indazol-4-yl)phenyl)-2-methoxynicotinaldehyde

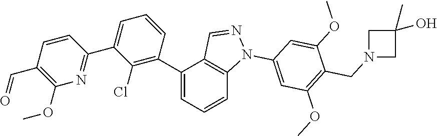 [SMILES: COc1cc(-n2ncc3c(-c4cccc(-c5ccc(C=O)c(OC)n5)c4Cl)cccc32)cc(OC)c1CN1CC(C)(O)C1]

A mixture of Intermediate C2 (388.0 mg, 1.04 mmol), 1-(4-(4-bromo-1H-indazol-1-yl)-2,6-dimethoxybenzyl)-3-methylazetidin-3-ol (299.0 mg, 0.69 mmol), Pd(PPh₃)₄ (80 mg, 0.069 mmol) and K₂CO₃ (287.0 mg, 2.08 mmol) in a mixture of 1,4-dioxane and H₂O (6:1) 14 mL was sparged with argon for 10 minutes before stirring at 95° C. for 1.5 hours under argon atmosphere. Then, the mixture was cooled down and diluted with brine. The resulting mixture was extracted 3 times with EtOAc and the organic layer was collected, dried over anhydrous Na₂SO₄, filtered, and evaporated to dryness. The crude mixture was purified by column chromatography (silica gel, gradient elution, 0 to 10% MeOH/DCM) to provide the desired product.

Step 3: 4-(((6-(2-chloro-3-(1-(4-((3-hydroxy-3-methylazetidin-1-yl)methyl)-3,5-dimethoxy phenyl)-1H-indazol-4-yl)phenyl)-2-methoxypyridin-3-yl)methyl)methyl)amino)bicyclo[2.2.2]octane-1-carboxylic acid

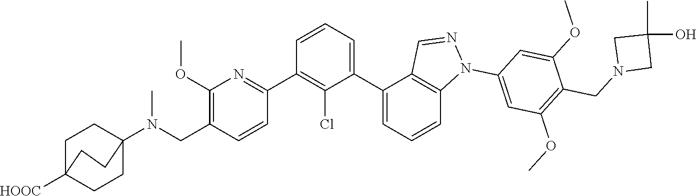 [SMILES: COc1cc(-n2ncc3c(-c4cccc(-c5ccc(CN(C)C67CCC(C(=O)O)(CC6)CC7)c(OC)n5)c4Cl)cccc32)cc(OC)c1CN1CC(C)(O)C1]

NEt₃ was added to a mixture of 4-aminobicyclo[2.2.2]octane-1-carboxylic acid hydrochloride (29.7 mg, 0.18 mmol) in 5 mL MeOH/DCM (1:1) until the pH was around 7. Then, the mixture was transferred to a mixture of 6-(2-chloro-3-(1-(4-((3-hydroxy-3-methylazetidin-1-yl)methyl)-3,5-dimethoxyphenyl)-1H-indazol-4-yl)phenyl)-2-methoxynicotinaldehyde (71.6 mg, 0.18 mmol) in 5 mL MeOH/DCM (1:1) at room temperature. After that, acetic acid was added until the pH was around 4. After the reaction was stirred for 1 hour, sodium triacetoxyborohydride (74.2 mg, 0.35 mmol) was added. After the reaction was completed, 37% formaldehyde solution (1 mL) and sodium cyanoborohydride (22.0 mg, 0.35 mmol) were added, respectively. The reaction was quenched by a small amount of MeOH and evaporated to dryness. The crude mixture was purified by column chromatography (silica gel, gradient elution, 0 to 30% MeOH/DCM). The fractions containing the product were collected and the solvent was removed under reduced pressure, resulting the title compound. LCMS Method F, RT=9.822 min, m/z=766.3 [M+H]⁺, LCMS Method D, RT=3.699 min, m/z=766.3358 [M+H]⁺, exact mass: 765.3293.

¹H NMR (500 MHz, MeOD-d₄) δ ppm 8.03 (d, J=0.8 Hz, 1H), 7.93 (d, J=8.6 Hz, 1H), 7.84 (d, J=7.5 Hz, 1H), 7.68 (dd, J=7.2, 2.2 Hz, 1H), 7.62 (dd, J=8.6, 7.1 Hz, 1H), 7.59-7.52 (m, 2H), 7.33 (d, J=7.5 Hz, 1H), 7.29 (d, J=7.1 Hz, 1H), 7.09 (s, 2H), 4.09 (s, 2H), 4.05 (s, 3H), 3.96 (s, 6H), 3.63-3.53 (m, 4H), 3.36-3.32 (m, 3H), 2.44 (s, 2H), 1.99-1.85 (m, 12H), 1.47 (s, 3H).

Example 157: (R)-4-(((7-cyano-2-(3-(1-(4-((3-hydroxypyrrolidin-1-yl)methyl)-3,5-dimethoxyphenyl)-1H-indazol-4-yl)-2-methylphenyl)benzo[d]oxazol-5-yl)methyl)(methyl)amino)bicyclo[2.2.2]octane-1-carboxylic acid

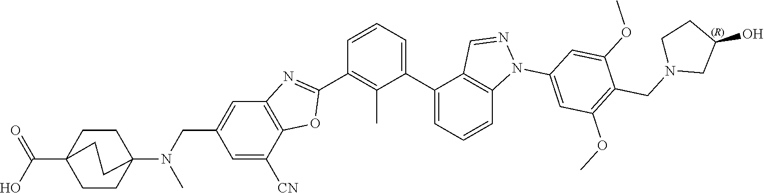

The preparation of intermediate (R)-5-formyl-2-(3-(1-(4-((3-hydroxypyrrolidin-1-yl)methyl)-3,5-dimethoxyphenyl)-1H-indazol-4-yl)-2-methylphenyl)benzo[d]oxazole-7-carbonitrile was described in Example 154. After obtaining the desired intermediate, NEt₃ was added to a solution of 4-aminobicyclo[2.2.2]octane-1-carboxylic acid hydrochloride (69.40 mg, 0.38 mmol) in 10 mL MeOH/DCM (1:1) until the pH was around 7. Then, the mixture was transferred to a mixture of (R)-5-formyl-2-(3-(I-(4-((3-hydroxypyrrolidin-1-yl)methyl)-3,5-dimethoxyphenyl)-1H-indazol-4-yl)-2-methylphenyl)benzo[d]oxazole-7-carbonitrile (80.00 mg, 0.10 mmol) in 10 mL MeOH/DCM (1:1) at room temperature. After that, acetic acid was added until the pH was around 4. After the reaction was stirred for 1 hour, sodium triacetoxyborohydride (105 mg, 0.50 mmol) was added. After the reaction was completed, 37% formaldehyde solution (1 mL) and sodium cyanoborohydride (62.40 mg, 0.10 mmol) were added, respectively. The reaction was quenched by adding a small amount of MeOH and evaporated to dryness. The crude mixture was purified by column chromatography (silica gel, gradient elution, 30 to 50% MeOH/DCM). The fractions containing the product were collected and the solvent was removed under reduced pressure, resulting the title compound. LCMS Method G, RT=10.147 min, m/z=781.3 [M+H]⁺, LCMS Method D, RT=3.761 min, m/z=781.3703 [M+H]⁺, exact mass: 780.3635. ¹H NMR (500 MHz, MeOD-d₄) δ ppm 8.27-8.24 (m, 1H), 8.00 (s, 2H), 7.97 (d, J=8.6 Hz, 1H), 7.74 (s, 1H), 7.66 (dd, J=8.6, 7.0 Hz, 1H), 7.55-7.54 (m, 2H), 7.24 (d, J=7.1 Hz, 1H), 7.16 (s, 2H), 4.52-4.48 (m, 1H), 4.31 (s, 2H), 4.01 (s, 9H), 3.71 (s, 2H), 2.57 (s, 3H), 2.31 (t, J=7.3 Hz, 1H), 2.28-2.21 (m, 2H), 1.98-1.93 (m, 2H), 1.87-1.84 (m, 6H), 1.73-1.70 (m, 6H), 1.62-1.55 (m, 2H).

Example 158: (R)-1-(4-(4-(3-((2-(difluoromethyl)-7-(((R)-3-hydroxypyrrolidin-1-yl)methyl)pyrido[3,2-d]pyrimidin-4-yl)amino)-2-methylphenyl)-1H-indazol-1-yl)-2,6-dimethoxybenzyl)-3-methylpyrrolidine-3-carboxylic acid

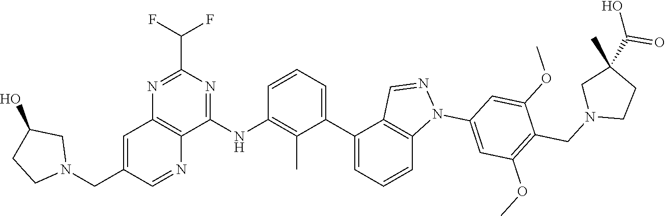 [SMILES: COc1cc(-n2ncc3c(-c4cccc(Nc5nc(C(F)F)nc6cc(CN7CC[C@@H](O)C7)cnc56)c4C)cccc32)cc(OC)c1CN1CC[C@@](C)(C(=O)O)C1]

A mixture of Intermediate K (30.0 mg, 0.045 mmol) and (R)-3-methylpyrrolidine-3-carboxylic acid (8.5 mg, 0.066 mmol) in MeOH/DCM (1:1, 10 mL) was stirred at room temperature and triethylamine (0.10 ml) was added. The reaction was stirred for 2 hours and sodium triacetoxyborohydride (40.0 mg, 0.19 mmol) was added and the mixture was stirred for additional 2 hours. The mixture was evaporated to dryness and purified by column chromatography (silica gel, gradient elution, 10 to 80%/o MeOH/DCM) to provide the title compound. LCMS Method G, RT=9.383 min, m/z=779.4 [M+H]⁺, LCMS Method D, RT=3.677 min, m/z=779.3488 [M+H]⁺, exact mass: 778.3403. ¹H NMR (500 MHz, MeOD-d₄) δ ppm 8.95 (s, 1H), 8.16 (s, 1H), 8.02 (s, 1H), 7.91 (d, J=7.95 Hz, 2H), 7.61 (t, J=7.70 Hz, 1H), 7.42 (t, J=7.79 Hz, 1H), 7.28 (d, J=7.57 Hz, 1H), 7.21 (d, J=7.11 Hz, 1H), 7.13 (s, 2H), 6.57 (t, J=54.88 Hz, 11H), 4.45 (s, 2H), 4.35 (m, 1H), 3.99 (s, 6H), 3.99 (m, 1H), 3.92 (d, J=13.81 Hz, 1H), 3.87 (d, J=13.70 Hz, 1H), 2.84-2.79 (m, 2H), 2.58-2.53 (m, 2H), 2.19-2.12 (m, 2H), 2.14 (s, 3H), 1.76-1.70 (m, 1H), 1.33-1.25 (m, 3H), 1.31 (s, 3H), 0.89-0.82 (m, 1H).

Example 159: 4-(((7-cyano-2-(3-(1-(4-((3-hydroxyazetidin-1-yl)methyl)-3,5-dimethoxyphenyl)-1H-indazol-4-yl)-2-methylphenyl)benzo[d]oxazol-5-yl)methyl)(methyl)amino)bicyclo[2.2.2]octane-1-carboxylic acid

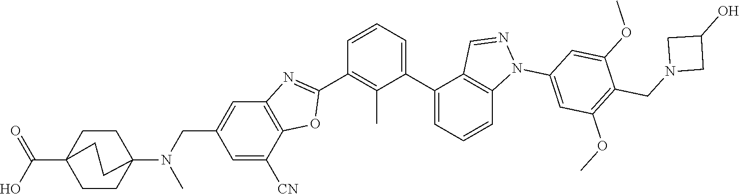 [SMILES: COc1cc(-n2ncc3c(-c4cccc(-c5nc6cc(CN(C)C78CCC(C(=O)O)(CC7)CC8)cc(C#N)c6o5)c4C)cccc32)cc(OC)c1CN1CC(O)C1]

### Step 1: 1-(4-(4-bromo-1H-indazol-1-yl)-2,6-dimethoxybenzyl)azetidin-3-ol

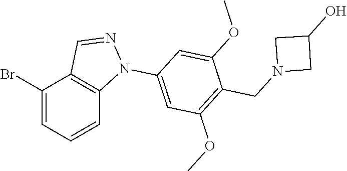 [SMILES: COc1cc(-n2ncc3c(Br)cccc32)cc(OC)c1CN1CC(O)C1]

NEt₃ was added to a solution of azetidin-3-ol hydrochloride (60.70 mg, 0.55 mmol) in 10 mL MeOH/DCM (1:1) until the pH was around 7. Then, the mixture was transferred to a mixture of Intermediate B4 (200.00 mg, 0.55 mmol) in 10 mL MeOH/DCM (1:1) and acidified with acetic acid (pH around 4). After the reaction was stirred for 1 hour, sodium cyanoborohydride (104 mg, 1.66 mmol) was added. The reaction was quenched by adding a small amount of MeOH and evaporated to dryness. The crude mixture was purified by column chromatography (silica gel, gradient elution, 0 to 10% MeOH/DCM) to provide the product Step 2: 1-(4-(4-(3-(7-chloro-5-(hydroxymethyl)benzo[d]oxazol-2-yl)-2-methylphenyl)-1H-indazol-1-yl)-2,6-dimethoxybenzyl)azetidin-3-ol

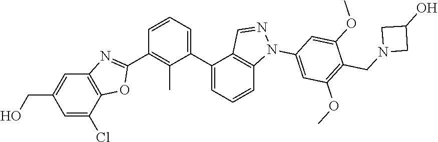 [SMILES: COc1cc(-n2ncc3c(-c4cccc(-c5nc6cc(CO)cc(Cl)c6o5)c4C)cccc32)cc(OC)c1CN1CC(O)C1]

A mixture of Intermediate G-1 (287 mg, 0.72 mmol), 1-(4-(4-bromo-1H-indazol-1-yl)-2,6-dimethoxybenzyl)azetidin-3-ol (300 mg, 0.72 mmol), Pd(PPh₃)₄ (83.00 mg, 0.07 mmol) and K₂CO₃ (297 mg, 2.15 mmol) in a mixture of 1,4-dioxane and H₂O (6:1) 20 mL was purged with argon for 10 minutes before stirring at 110° C. for 2 hours under argon atmosphere. Then, the mixture was cooled and diluted with brine. The resulting mixture was extracted three times with EtOAc and the organic layer was collected, dried over anhydrous Na₂SO₄, filtered, and evaporated to dryness. The crude mixture was purified by column chromatography (silica gel, gradient elution, 0 to 20% MeOH/DCM) to provide the product.

### Step 3: 2-(3-(1-(4-((3-hydroxyazetidin-1-yl)methyl)-3,5-dimethoxyphenyl)-1H-indazol-4-yl)-2-methylphenyl)-5-(hydroxymethyl)benzo[d]oxazole-7-carbonitrile

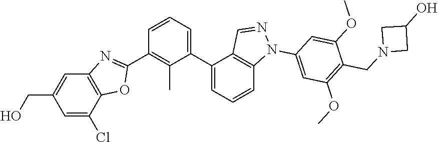 [SMILES: COc1cc(-n2ncc3c(-c4cccc(-c5nc6cc(CO)cc(Cl)c6o5)c4C)cccc32)cc(OC)c1CN1CC(O)C1]

A mixture of 1-(4-(4-(3-(7-chloro-5-(hydroxymethyl)benzo[d]oxazol-2-yl)-2-methylphenyl)-1H-indazol-1-yl)-2,6-dimethoxybenzyl)azetidin-3-ol (100 mg, 0.16 mmol), K₄[Fe(CN)₆]3H₂O (346 mg, 0.82 mmol), tBuXPhos Pd G3 (13.00 mg, 0.02 mmol) and KOAc (56.20 mg, 0.57 mmol) in a mixture of THF and H₂O (1:1) 10 mL was purged with nitrogen for 10 minutes before stirring at 90° C. for 3 hours under nitrogen atmosphere. Then, the mixture was cooled and diluted with brine. The resulting mixture was extracted three times with EtOAc and the organic layer was collected, dried over anhydrous Na₂SO₄, filtered, and evaporated to dryness. The crude mixture was purified by column chromatography (silica gel, gradient elution, 30 to 50% MeOH/DCM) to provide the product.

### Step 4: 5-formyl-2-(3-(I-(4-((3-hydroxyazetidin-1-yl)methyl)-3,5-dimethoxyphenyl)-1H-indazol-4-yl)-2-methylphenyl)benzo[d]oxazole-7-carbonitrile

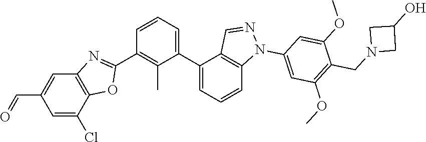 [SMILES: COc1cc(-n2ncc3c(-c4cccc(-c5nc6cc(C=O)cc(Cl)c6o5)c4C)cccc32)cc(OC)c1CN1CC(O)C1]

A solution of Dess-Martin periodinane (317 mg, 0.75 mmol), and pyridine (6 to 7 drops) in DCM 5 mL was added to a solution of 2-(3-(1-(4-((3-hydroxyazetidin-1-yl)methyl)-3,5-dimethoxyphenyl)-1H-indazol-4-yl)-2-methylphenyl)-5-(hydroxymethyl)benzo[d]oxazole-7-carbonitrile (150 mg, 0.25 mmol), in DCM 5 mL at rt. The reaction was stirred for 30 minutes, evaporated to dryness, and purified by column chromatography (silica gel, gradient elution, 0 to 15% MeOH/DCM) to provide the product.

### Step 5: 4-(((7-cyano-2-(3-(1-(4-((3-hydroxyazetidin-1-yl)methyl)-3,5-dimethoxyphenyl)-1H-indazol-4-yl)-2-methylphenyl)benzo[d]oxazol-5-yl)methyl)(methyl)amino)bicyclo[2.2.2]octane-1-carboxylic acid

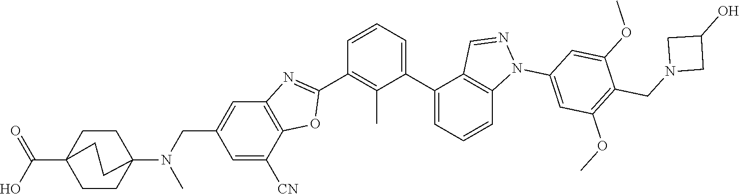 [SMILES: COc1cc(-n2ncc3c(-c4cccc(-c5nc6cc(CN(C)C78CCC(C(=O)O)(CC7)CC8)cc(C#N)c6o5)c4C)cccc32)cc(OC)c1CN1CC(O)C1]

This step was prepared using similar procedures as described in the Example 157. The crude mixture was purified by column chromatography (silica gel, gradient elution, 30 to 50% MeOH/DCM). The fractions containing the product were collected and the solvent was removed under reduced pressure, resulting the title compound. LCMS Method G, RT=10.028 min, m/z=767.3 [M+H]⁺, LCMS Method D, RT=3.761 min, m/z=767.3540 [M+H]⁺, exact mass: 766.3479. ¹H NMR (500 MHz, CDCl₃) δ ppm 8.56 (s, 1H), 8.32 (d, J=7.8 Hz, 1H), 8.04-8.01 (m, 1H), 7.97 (d, J=1.1 Hz, 1H), 7.68-7.63 (m, 1H), 7.53 (s, 3H), 7.18 (dd, J=7.0, 0.8 Hz, 2H), 6.97 (s, 1H), 4.86-4.82 (m, 1H), 4.71-4.66 (m, 1H), 4.65-4.61 (m, 1H), 4.42-4.39 (m, 1H), 4.33-4.29 (m, 1H), 4.24-4.19 (m, 2H), 4.01 (s, 3H), 3.98 (s, 3H), 3.93 (s, 3H), 3.67 (s, 2H), 2.66 (s, 3H), 2.53-2.47 (m, 6H), 2.31-2.26 (m, 6H).

Example 160: 4-(((2′-chloro-3′-(1-(3,5-dimethoxy-4-((7-oxo-2,6-diazaspiro[3.4]octan-2-yl)methyl)phenyl)-1H-indazol-4-yl)-3-fluoro-5-methoxy-[1,1′-biphenyl]-4-yl)methyl)(methyl)amino)bicyclo[2.2.2]octane-1-carboxylic acid

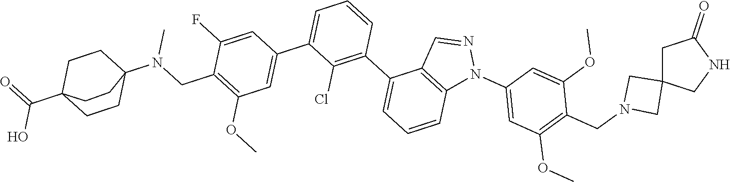 [SMILES: COc1cc(-c2cccc(-c3cccc4c3cnn4-c3cc(OC)c(CN4CC5(CNC(=O)C5)C4)c(OC)c3)c2Cl)cc(F)c1CN(C)C12CCC(C(=O)O)(CC1)CC2]

### Step 1: 2-fluoro-6-methoxy-4-(4,4,5,5-tetramethyl-1,3,2-dioxaborolan-2-yl)benzaldehyde

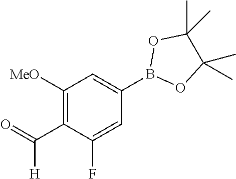 [SMILES: COc1cc(B2OC(C)(C)C(C)(C)O2)cc(F)c1C=O]

Pd(dppf)Cl₂·CH₂Cl₂ (0.14 g, 0.18 mmol) was added to a solution of 4-bromo-2-fluoro-6-methoxybenzaldehyde (0.41 g, 1.75 mmol), B₂Pin₂ (0.49 g, 1.92 mmol), and KOAc (0.52 g, 5.25 mmol) in 1,2-dimethoxyethane 20 mL. The resulting suspension was sparged with argon for 10 min. After the reaction was sealed and stirred at 80° C. for 2 hours under argon atmosphere, it was cooled down and diluted with water. The resulting mixture was extracted 3 times with EtOAc and the organic layer was collected, dried over anhydrous Na₂SO₄, filtered, and evaporated to dryness. The desired product was further used without purification.

### Step 2: 3′-bromo-2′-chloro-3-fluoro-5-methoxy-[1,1′-biphenyl]-4-carbaldehyde

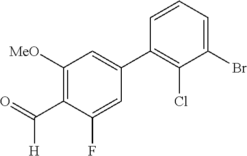 [SMILES: COc1cc(-c2cccc(Br)c2Cl)cc(F)c1C=O]

Pd(PPh₃)₄ (0.20 g, 0.18 mmol) was added to a solution of 1,3-dibromo-2-chlorobenzene (0.71 g, 2.63 mmol) and 2-fluoro-6-methoxy-4-(4,4,5,5-tetramethyl-1,3,2-dioxaborolan-2-yl)benzaldehyde (0.49 g, 1.75 mmol) in 1,4-dioxane 12 mL. A solution of K₂CO₃ (0.72 g, 5.25 mmol) in H₂O 2 mL was added to the reaction mixture before it was sparged with argon for 10 min. After the reaction mixture was stirred at 95° C. for 3 hours under argon atmosphere, it was cooled down and diluted with brine. The resulting mixture was extracted 3 times with EtOAc and the organic layer was collected, dried over anhydrous Na₂SO₄, filtered, and evaporated to dryness. The crude mixture was purified by column chromatography (silica gel, gradient elution, 0 to 20% EtOAc/hexanes) to provide a desired product.

### Step 3: 4-(((3′-bromo-2′-chloro-3-fluoro-5-methoxy-[1,1′-biphenyl]-4-yl)methyl)(methyl)amino)bicyclo[2.2.2]octane-1-carboxylic acid

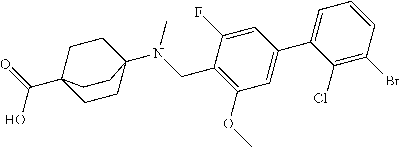 [SMILES: COc1cc(-c2cccc(Br)c2Cl)cc(F)c1CN(C)C12CCC(C(=O)O)(CC1)CC2]

NEt₃ was added to a mixture of 4-aminobicyclo[2.2.2]octane-1-carboxylic acid hydrochloride (0.090 g, 0.437 mmol) in 2 mL MeOH/DCM (1:1) until the pH was around 7. Then, the mixture was transferred to 3′-bromo-2′-chloro-3-fluoro-5-methoxy-[1,1′-biphenyl]-4-carbaldehyde (0.15 g, 0.437 mmol), acidified with acetic acid until the pH was around 4. After the reaction was stirred for 1 hour, sodium triacetoxyborohydride (0.463 g, 2.18 mmol) was added. After the reaction was completed, 37% formaldehyde solution (1 mL) and sodium cyanoborohydride (an excess amount) were added, respectively. The reaction was quenched by adding a small amount of MeOH and evaporated to dryness. The crude mixture was purified by column chromatography (silica gel, gradient elution, 20 to 100% MeOH/DCM) to provide the product.

### Step 4: 4-(((2′-chloro-3-fluoro-β′-(1-(4-formyl-3,5-dimethoxyphenyl)-1H-indazol-4-yl)-5-methoxy-[1,1′-biphenyl]-4-yl)methyl)(methyl)amino)bicyclo[2.2.2]octane-1-carboxylic acid

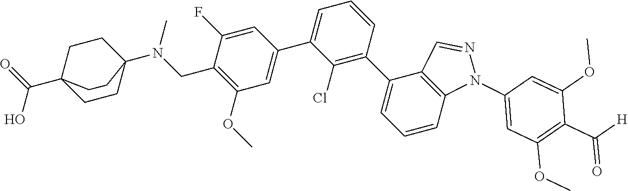 [SMILES: COc1cc(-n2ncc3c(-c4cccc(-c5cc(F)c(CN(C)C67CCC(C(=O)O)(CC6)CC7)c(OC)c5)c4Cl)cccc32)cc(OC)c1C=O]

A mixture of Intermediate B4-1 (50 mg, 0.121 mmol), 4-(((3′-bromo-2′-chloro-3-fluoro-5-methoxy-[1,1′-biphenyl]-4-yl)methyl)(methyl)amino)bicyclo[2.2.2]octane-1-carboxylic acid (62 mg, 0.121 mmol), Pd(PPh₃)₄ (14 mg, 0.012 mmol) and K₂CO₃ (59 mg, 0.425 mmol) in a mixture of 1,4-dioxane and H₂O (6:1) 10 mL was purged with argon for 10 mins before stirring at 110° C. for 1 hour under argon atmosphere. Then, the mixture was cooled and diluted with H₂O. The resulting mixture was extracted three times with DCM and the organic layer was collected, dried over anhydrous Na₂SO₄, filtered, and evaporated to dryness. The crude mixture was purified by column chromatography (silica gel, gradient elution, 20 to 100% MeOH/DCM) to provide the product.

Step 5: 4-(((2′-chloro-3′-(1-(3,5-dimethoxy-4-((7-oxo-2,6-diazaspiro[3.4]octan-2-yl)methyl)phenyl)-1H-indazol-4-yl)-3-fluoro-5-methoxy-[1,1′-biphenyl]-4-yl)methyl)(methyl)amino)bicyclo[2.2.2]octane-1-carboxylic acid

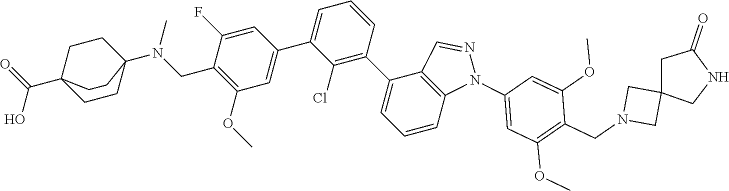 [SMILES: COc1cc(-c2cccc(-c3cccc4c3cnn4-c3cc(OC)c(CN4CC5(CNC(=O)C5)C4)c(OC)c3)c2Cl)cc(F)c1CN(C)C12CCC(C(=O)O)(CC1)CC2]

NEt₃ was added to a mixture 2,6-diazaspiro[3.4]octan-7-one; bis(4-methylbenzene-1-sulfonic acid) (13.2 mg, 0.028 mmol) in 2 mL MeOH/DCM (1:1) until the pH was around 7. Then, the mixture was transferred to 4-(((2′-chloro-3-fluoro-β′-(1-(4-formyl-3,5-dimethoxyphenyl)-1H-indazol-4-yl)-5-methoxy-[1,1′-biphenyl]-4-yl)methyl)(methyl)amino)bicyclo[2.2.2]octane-1-carboxylic acid (20 mg, 0.028 mmol) at room temperature. After that, acetic acid was added until the pH was around 4. After the reaction was stirred for 1 hour, sodium bis(acetyloxy)boranuidyl acetate (30 mg, 0.140 mmol) was added. The complete reaction was quenched by adding a small amount of MeOH and evaporated to dryness. The crude mixture was purified by column chromatography (silica gel, 70% MeOH/DCM). The fractions containing the product were collected and the solvent was removed under reduced pressure, resulting the title compound. LCMS Method G, RT=9.905 min, m/z=822.3 [M+H]⁺, LCMS Method D, RT=3.731 min, m/z=822.3425 [M+H]⁺, exact mass: 821.3355. ¹H NMR (500 MHz, Methanol-d₄) δ 8.01 (s, 1H), 7.90 (d, J=8.6 Hz, 1H), 7.60 (dd, J=8.6, 7.1 Hz, 1H), 7.55-7.53 (m, 2H), 7.52-7.49 (m, 1H), 7.25 (d, J=7.0 Hz, 1H), 7.08 (s, 1H), 7.06 (s, 1H), 7.02 (dd, J=9.9, 1.4 Hz, 1H), 4.06 (s, 1H), 3.97 (s, 3H), 3.94 (s, 6H), 3.72 (s, 3H), 3.57 (s, 2H), 3.14 (q, J=7.3 Hz, 4H), 2.64 (s, 3H), 2.58 (s, 2H), 2.54 (s, 1H), 2.44 (s, 1H), 2.01-1.93 (m, 11H), 1.89 (s, 2H).

Example 161: 4-(((6-(2-chloro-3-(1-(4-(((trans-3-hydroxycyclobutyl)(methyl)amino) methyl)-3,5-dimethoxyphenyl)-1H-indazol-4-yl)phenyl)-2-methoxypyridin-3-yl)methyl) (methyl)amino)bicyclo[2.2.2]octane-1-carboxylic acid

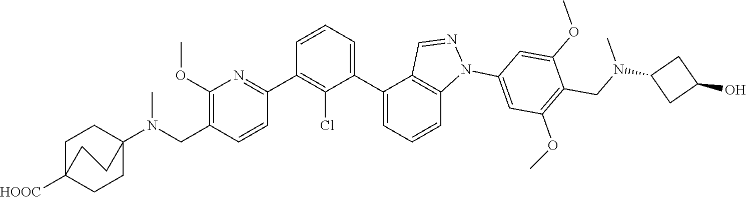 [SMILES: COc1cc(-n2ncc3c(-c4cccc(-c5ccc(CN(C)C67CCC(C(=O)O)(CC6)CC7)c(OC)n5)c4Cl)cccc32)cc(OC)c1CN(C)[C@H]1C[C@H](O)C1]

### Step 1: 6-(2-chloro-3-(1-(4-(((trans-3-hydroxycyclobutyl)(methyl)amino)methyl)-3,5-dimethoxy phenyl)-1H-indazol-4-yl)phenyl)-2-methoxynicotinaldehyde

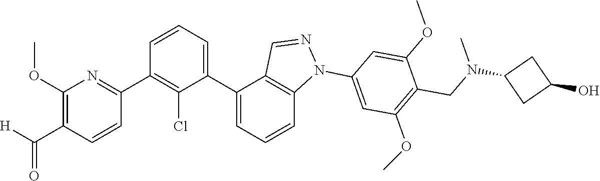 [SMILES: COc1cc(-n2ncc3c(-c4cccc(-c5ccc(C=O)c(OC)n5)c4Cl)cccc32)cc(OC)c1CN(C)[C@H]1C[C@H](O)C1]

A mixture of Intermediate C2 (145 mg, 0.39 mmol), Intermediate B4-5 (133.0 mg, 0.30 mmol), Pd(PPh₃)₄ (34.4 mg, 0.03 mmol) and K₂CO₃ (124.0 mg, 0.89 mmol) in a mixture of 1,4-dioxane and H₂O (6:1) 14 mL was sparged with argon for 10 minutes before stirring at 95° C. for 1.5 hours under argon atmosphere. Then, the mixture was cooled down and diluted with brine. The resulting mixture was extracted 3 times with EtOAc and the organic layer was collected, dried over anhydrous Na₂SO₄, filtered, and evaporated to dryness. The crude mixture was purified by column chromatography (silica gel, gradient elution, 0 to 10% MeOH/DCM) to provide the desired product.

Step 2: 4-(((6-(2-chloro-3-(1-(4-(((trans-3-hydroxycyclobutyl)(methyl)amino)methyl)-3,5-dimethoxyphenyl)-1H-indazol-4-yl)phenyl)-2-methoxypyridin-3-yl)methyl)(methyl)amino) bicyclo[2.2.2]octane-1-carboxylic acid

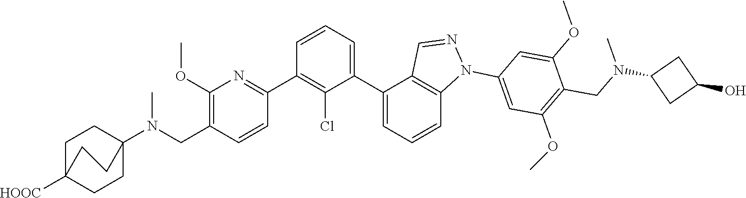 [SMILES: COc1cc(-n2ncc3c(-c4cccc(-c5ccc(CN(C)C67CCC(C(=O)O)(CC6)CC7)c(OC)n5)c4Cl)cccc32)cc(OC)c1CN(C)[C@H]1C[C@H](O)C1]

NEt₃ was added to a mixture of 4-aminobicyclo[2.2.2]octane-1-carboxylic acid hydrochloride (48.7 mg, 0.288 mmol) in 5 mL MeOH/DCM (1:1) until the pH was around 7. Then, the mixture was transferred to a mixture of 6-(2-chloro-3-(1-(4-(((trans-3-hydroxycyclobutyl)(methyl)amino)methyl)-3,5-dimethoxyphenyl)-1H-indazol-4-yl)phenyl)-2-methoxynicotinaldehyde (118.0 mg, 0.192 mmol) in 5 mL MeOH/DCM (1:1) at room temperature. After that, acetic acid was added until the pH was around 4. After the reaction was stirred for 1 hour, sodium triacetoxyborohydride (202.9 mg, 0.96 mmol) was added. After the reaction was completed, 37% formaldehyde solution (1 mL) and sodium cyanoborohydride (60.3 mg, 0.96 mmol) were added, respectively. The complete reaction was quenched by adding a small amount of MeOH and evaporated to dryness. The crude mixture was purified by column chromatography (silica gel, gradient elution, 0 to 30% MeOH/DCM). The fractions containing the product were collected and the solvent was removed under reduced pressure, resulting the title compound. LCMS Method G, Rt=9.687 min, m/z=780.3 [M+H]⁺, LCMS Method D, RT=3.700 min, m/z=780.3566 [M+H]⁺, exact mass: 779.3450. ¹H NMR (500 MHz, MeOD-d₄) δ ppm 8.05 (d, J=0.7 Hz, 11H), 7.95 (d, J=8.6 Hz, 11H), 7.89 (d, J=7.5 Hz, 1H), 7.69 (dd, J=7.2, 2.2 Hz, 1H), 7.64 (dd, J=8.6, 7.2 Hz, 11H), 7.60-7.53 (m, 2H), 7.37 (d, J=7.5 Hz, 1H), 7.30 (d, J=7.0 Hz, 1H), 7.13 (s, 2H), 4.47-4.30 (m, 1H), 4.07 (d, J=6.0 Hz, 3H), 4.04 (t, J=6.6 Hz, 111), 4.02-3.98 (m, 6H), 3.81 (br, 1H), 2.60-2.54 (m, 2H), 2.51 (q, J=7.0 Hz, 4H), 2.38-2.22 (m, 2H), 1.96 (s, 12H), 1.33-1.28 (m, 5H).

### Example 162: (S)-5-(((4-(4-(3-(5-((6-acetyl-2,6-diazaspiro[3.3]heptan-2-yl)methyl)-6-methoxypyridin-2-yl)-2-chlorophenyl)-1H-indazol-1-yl)-2,6-dimethoxybenzyl)amino)methyl)pyrrolidin-2-one

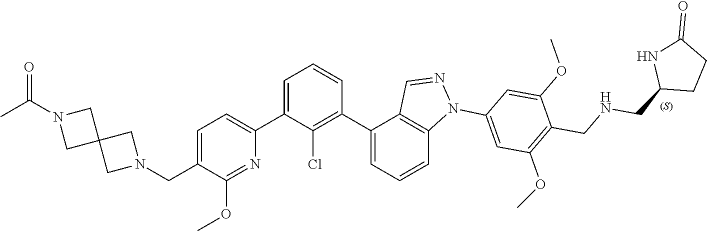

### Step 1: 1-(6-((6-(3-bromo-2-chlorophenyl)-2-methoxypyridin-3-yl)methyl)-2,6-diazaspiro[3.3]heptan-2-yl)ethan-1-one

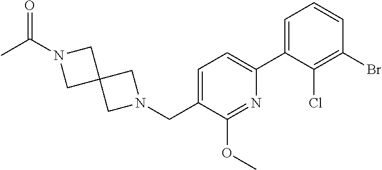 [SMILES: COc1nc(-c2cccc(Br)c2Cl)ccc1CN1CC2(C1)CN(C(C)=O)C2]

NEt₃ was added to a solution of 1-{2,6-diazaspiro[3.3]heptan-2-yl}ethan-1-one trifluoroacetic acid (0.23 g, 0.92 mmol) in 6 mL MeOH/DCM (1:1) until the pH was around 7. Then, the mixture was transferred to a mixture of Intermediate C1 (0.15 g, 0.46 mmol) in 6 mL MeOH/DCM (1:1) at room temperature. After that, acetic acid was added until the pH was around 4. After the reaction was stirred for 1 hour, sodium cyanoborohydride (0.15 g, 2.30 mmol) was added. The reaction was stirred at room temperature for an additional 2.5 hours, quenched by adding a small amount of MeOH and evaporated to dryness. The crude mixture was purified by column chromatography (silica gel, gradient elution, 0 to 15% MeOH/DCM) to provide the desired product.

### Step 2: 4-(4-(3-(5-((6-acetyl-2,6-diazaspiro[3.3]heptan-2-yl)methyl)-6-methoxypyridin-2-yl)-2-chlorophenyl)-1H-indazol-1-yl)-2,6-dimethoxybenzaldehyde

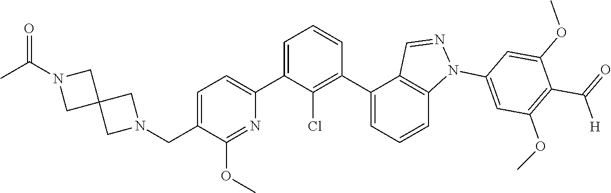 [SMILES: COc1cc(-n2ncc3c(-c4cccc(-c5ccc(CN6CC7(C6)CN(C(C)=O)C7)c(OC)n5)c4Cl)cccc32)cc(OC)c1C=O]

A mixture of 1-(6-((6-(3-bromo-2-chlorophenyl)-2-methoxypyridin-3-yl)methyl)-2,6-diazaspiro[3.3]heptan-2-yl)ethan-1-one (0.22 g, 0.48 mmol), Intermediate B4-1 (0.24 g, 0.58 mmol), Pd(PPh₃)₄ (0.055 g, 0.048 mmol) and K₂CO₃ (0.20 g, 1.44 mmol) in a mixture of 1,4-dioxane and H₂O (6:1) 14 mL was purged with argon for 10 minutes and stirred at 95° C. for 3 hours under argon atmosphere. Then, the mixture was cooled and diluted with brine. The resulting mixture was extracted three times with EtOAc and the organic layer was collected, dried over anhydrous Na₂SO₄, filtered, and evaporated to dryness. The crude mixture was purified by column chromatography (silica gel, gradient elution, 0 to 15% MeOH/DCM) to provide the desired product.

### Step 3: (S)-5-(((4-(4-(3-(5-((6-acetyl-2,6-diazaspiro[3.3]heptan-2-yl)methyl)-6-methoxypyridin-2-yl)-2-chlorophenyl)-1H-indazol-1-yl)-2,6-dimethoxybenzyl)amino)methyl)pyrrolidin-2-one

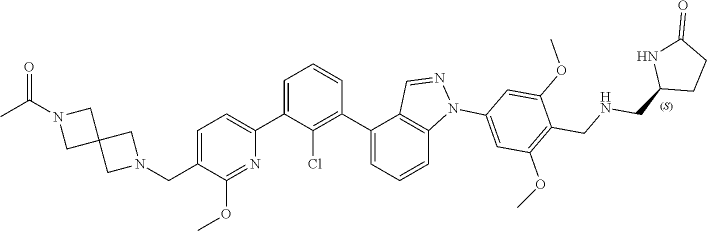

A mixture of 4-(4-(3-(5-((6-acetyl-2,6-diazaspiro[3.3]heptan-2-yl)methyl)-6-methoxypyridin-2-yl)-2-chlorophenyl)-1H-indazol-1-yl)-2,6-dimethoxybenzaldehyde (130 mg, 0.20 mmol) and (S)-5-(aminomethyl)pyrrolidin-2-one (22.4 mg, 0.24 mmol) in 4 mL MeOH/DCM (1:1) was stirred at room temperature and acetic acid was added until the pH was around 4. After the reaction was stirred for 1 hour, sodium cyanoborohydride (62.8 mg, 1.00 mmol) was added and the reaction was stirred at room temperature for an additional 1 hour. The reaction mixture was purified by column chromatography (silica gel, gradient elution, 10 to 20% MeOH/DCM). The fractions containing the product were collected and the solvent was removed under reduced pressure, resulting the title compound. LCMS Method F, RT=6.855 min, m/z=750.3 [M+H]⁺, LCMS Method D, RT=3.404 min, m/z=750.3158 [M+H]⁺, exact mass: 749.3092. ¹H NMR (500 MHz, MeOD-d₄) δ 8.02 (s, 11H), 7.90 (d, J=8.6 Hz, 1H), 7.70-7.64 (m, 2H), 7.61 (t, J=7.8 Hz, 11H), 7.57-7.50 (m, 2H), 7.29-7.24 (m, 21H), 7.05 (s, 2H), 4.30 (s, 2H), 4.05 (s, 21), 4.01 (s, 3H), 3.95 (s, 8H), 3.89-3.81 (m, 11H), 3.68 (s, 2H), 3.52 (q, J=8.1 Hz, 4H), 2.69 (t, J=5.5 Hz, 2H), 2.36-2.29 (m, J=15.8, 8.4 Hz, 2H), 1.84 (s, 3H), 1.34-1.24 (m, 2H).

Example 163: (R)-1-(4-(4-(3-((2-(difluoromethyl)-7-((3-hydroxypyrrolidin-1-yl)methyl)pyrido[3,2-d]pyrimidin-4-yl)amino)-2-methylphenyl)-1H-indazol-1-yl)-2,6-dimethoxybenzyl)piperidine-4-carboxylic acid

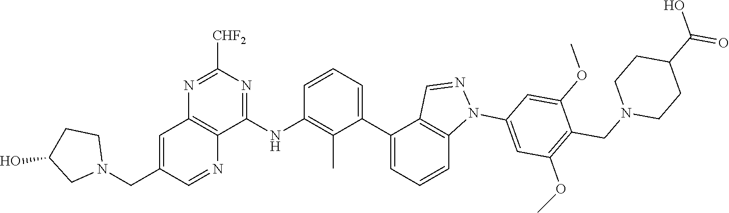 [SMILES: COc1cc(-n2ncc3c(-c4cccc(Nc5nc(C(F)F)nc6cc(CN7CC[C@@H](O)C7)cnc56)c4C)cccc32)cc(OC)c1CN1CCC(C(=O)O)CC1]

NEt₃ was added to a mixture of piperidine-4-carboxylic acid hydrochloride (28.2 mg, 0.17 mmol) in 5 mL of MeOH/DCM (1:1) until the pH was around 7. Then, the mixture was transferred to a mixture of Intermediate K (55.3 mg, 0.083 mmol) in 15 mL MeOH/DCM (1:1) and acidified with acetic acid (pH around 4). After the reaction was stirred for 1 hour, sodium triacetoxyborohydride (88.0 mg, 0.42 mmol) was added and the reaction was left until complete. The complete reaction was quenched by adding a small amount of MeOH and evaporated to dryness. The crude mixture was purified by column chromatography (silica gel, gradient elution, 15 to 100% MeOH/DCM). The fractions containing the product were collected and the solvent was removed under reduced pressure, resulting in the title compound. LCMS Method G, RT=8.939 min, m/z=779.3 [M+H]⁺, 801.3 [M+Na]⁺, LCMS Method D, RT=3.629 min, m/z=779.3485 [M+H]⁺, exact mass: 778.3403.

Example 164: (R)-4-(((2′-chloro-3-fluoro-β′-(1-(4-((3-hydroxypyrrolidin-1-yl)methyl)-3,5-dimethoxyphenyl)-1H-indazol-4-yl)-5-methoxy-[1,1′-biphenyl]-4-yl)methyl)(methyl)amino)bicyclo[2.2.2]octane-1-carboxylic acid

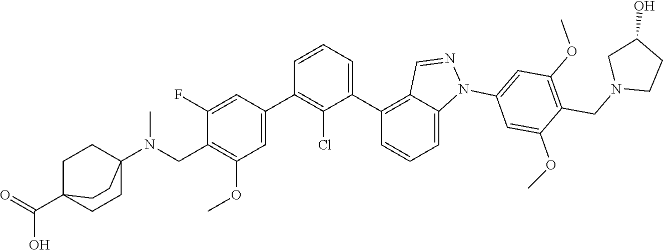 [SMILES: COc1cc(-c2cccc(-c3cccc4c3cnn4-c3cc(OC)c(CN4CC[C@@H](O)C4)c(OC)c3)c2Cl)cc(F)c1CN(C)C12CCC(C(=O)O)(CC1)CC2]

NEt₃ was added to a solution of (R)-pyrrolidin-3-ol hydrochloride (5.53 mg, 0.04 mmol) in 10 mL MeOH/DCM (1:1) until the pH was around 7. Then, the mixture was transferred to a mixture of 4-(((2′-chloro-3-fluoro-3′-(1-(4-formyl-3,5-dimethoxyphenyl)-1H-indazol-4-yl)-5-methoxy-[1,1′-biphenyl]-4-yl)methyl)(methyl)amino)bicyclo[2.2.2]octane-1-carboxylic acid (30.00 mg, 0.04 mmol, prepared by using similar procedures described in Example 160) in 5 mL MeOH/DCM (1:1) before adding acetic acid until the pH value around 4. After the reaction was stirred for 1 hour, sodium cyanoborohydride (12.8 mg g, 0.16 mmol) was added and the reaction was left until complete. The reaction was quenched by adding a small amount of MeOH and evaporated to dryness. The crude mixture was purified by column chromatography (silica gel, gradient elution, 20 to 70% MeOH/DCM). The fractions containing product were collected and the solvent was removed under reduced pressure, resulting the title compound. LCMS Method G, RT=10.083 min, m/z=783.3[M+H]⁺, LCMS Method D, RT=3.766 min, m/z=783.3316[M+H]⁺, exact mass: 782.3246. ¹H NMR (500 MHz, Methanol-d₄) δ 8.00 (s, 1H), 7.89 (d, J=6.2 Hz, 1H), 7.59 (dt, J=12.1, 6.1 Hz, 3H), 7.54-7.47 (m, 1H), 7.24 (t, J=6.3 Hz, 1H), 7.04 (s, 2H), 6.98 (s, 11H), 6.91 (dd, J=9.2, 1.4 Hz, 1H), 3.98 (s, 2H), 3.95-3.90 (m, 12H), 3.11-2.64 (m, 2H), 2.37 (m, 2H), 1.88 (m, 17H), 1.71 (dd, J=20.5, 12.5 Hz, 1H).

Example 165: (R)-1-((2-(difluoromethyl)-4-((3-(1-(4-(((R)-3-hydroxypyrrolidin-1-yl)methyl)-3,5-dimethoxyphenyl)-1H-indazol-4-yl)-2-methylphenyl)amino)pyrido[3,2-d]pyrimidin-7-yl)methyl)pyrrolidine-3-carboxylic acid

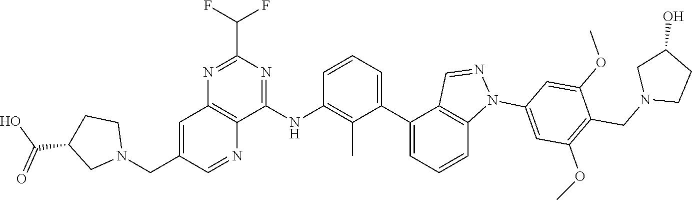 [SMILES: COc1cc(-n2ncc3c(-c4cccc(Nc5nc(C(F)F)nc6cc(CN7CC[C@@H](C(=O)O)C7)cnc56)c4C)cccc32)cc(OC)c1CN1CC[C@@H](O)C1]

### Step 1: (R)-1-((4-((3-bromo-2-methylphenyl)amino)-2-(difluoromethyl)pyrido[3,2-d]pyrimidin-7-yl)methyl)pyrrolidine-3-carboxylic acid

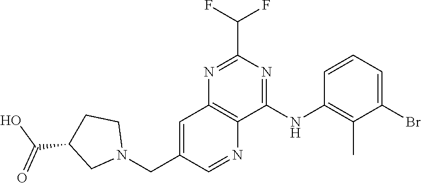 [SMILES: Cc1c(Br)cccc1Nc1nc(C(F)F)nc2cc(CN3CC[C@@H](C(=O)O)C3)cnc12]

A mixture of Intermediate 1 (150 mg, 0.38 mmol) and (R)-pyrrolidine-3-carboxylic acid (60.0 mg, 0.52 mmol) in MeOH:DCM (1:1, 15 mL) was acidified with acetic acid (pH around 4). After the reaction was stirred for 1 hour, sodium triacetoxyborohydride (0.25 g, 1.18 mmol) was added. The reaction was stirred at room temperature for overnight, evaporated to dryness, and purified by column chromatography (silica gel, gradient elution, 0 to 30% MeOH/DCM) to provide the title compound. LCMS Method B, Rt=3.237 min, m/z=492.1 [M+H]⁺, exact mass: 491.0768.

### Step 2: (R)-1-((2-(difluoromethyl)-4-((3-(1-(4-formyl-3,5-dimethoxyphenyl)-1H-indazol-4-yl)-2-methylphenyl)amino)pyrido[3,2-d]pyrimidin-7-yl)methyl)pyrrolidine-3-carboxylic acid

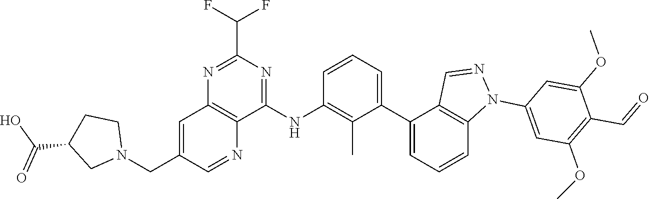 [SMILES: COc1cc(-n2ncc3c(-c4cccc(Nc5nc(C(F)F)nc6cc(CN7CC[C@@H](C(=O)O)C7)cnc56)c4C)cccc32)cc(OC)c1C=O]

A mixture of (R)-1-((4-((3-bromo-2-methylphenyl)amino)-2-(difluoromethyl)pyrido[3,2-d]pyrimidin-7-yl)methyl)pyrrolidine-3-carboxylic acid (0.19 g, 0.38 mmol), Intermediate B4-1 (0.24 g, 0.58 mmol), XPhos-Pd-G2 (0.033 g, 0.042 mmol) and K₃PO₄ (0.22 g, 1.04 mmol) in a mixture of 1,4-dioxane and H₂O (5:1) 12 mL was purged with argon for 10 min before it was stirred at 100° C. for 5 h under argon atmosphere. Then, the mixture was cooled and diluted with brine. The resulting mixture was extracted three times with EtOAc and the organic layer was collected, dried over anhydrous Na₂SO₄, filtered, and evaporated to dryness. The crude mixture was purified by column chromatography (silica gel, gradient elution, 0 to 50% MeOH/DCM) to provide the title compound (40.0 mg). LCMS Method B, Rt=3.477 min, m/z=694.3 [M+H]⁺, exact mass: 693.2511.

Step 3: (R)-1-((2-(difluoromethyl)-4-((3-(1-(4-(((R)-3-hydroxypyrrolidin-1-yl)methyl)-3,5-dimethoxyphenyl)-1H-indazol-4-yl)-2-methylphenyl)amino)pyrido[3,2-d]pyrimidin-7-yl)methyl)pyrrolidine-3-carboxylic acid

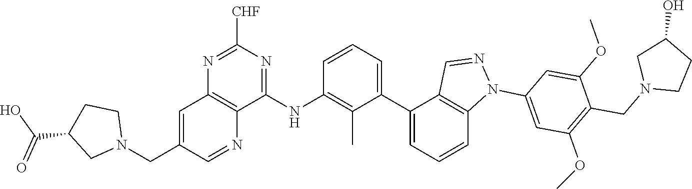

A mixture of (R)-1-((2-(difluoromethyl)-4-((3-(1-(4-formyl-3,5-dimethoxyphenyl)-1H-indazol-4-yl)-2-methylphenyl)amino)pyrido[3,2-d]pyrimidin-7-yl)methyl)pyrrolidine-3-carboxylic acid (40.0 mg, 0.08 mmol) and (R)-pyrrolidin-3-ol hydrochloride (13.2 mg, 0.11 mmol) in MeOH/DCM (1:2, 6 mL) was stirred at room temperature and triethylamine (0.10 ml) was added. The reaction was stirred for 2 hours and sodium triacetoxyborohydride (60.0 mg, 0.28 mmol) was added. The mixture was left for 2 hours, evaporated to dryness, and purified by column chromatography (silica gel, gradient elution, 50 to 80% MeOH/DCM) to provide the title compound. LCMS Method G, RT=8.492 min, m/z=765.3 [M+H]⁺, LCMS Method D, RT=3.592 min, m/z=765.3306 [M+H]⁺, exact mass: 764.3246.

Example 166: (R)-4-((4-(4-(3-((2-(difluoromethyl)-7-((3-hydroxypyrrolidin-1-yl)methyl)pyrido[3,2-d]pyrimidin-4-yl)amino)-2-methylphenyl)-1H-indazol-1-yl)-2,6-dimethoxybenzyl)(methyl)amino)bicyclo[2.2.2]octane-1-carboxylic acid

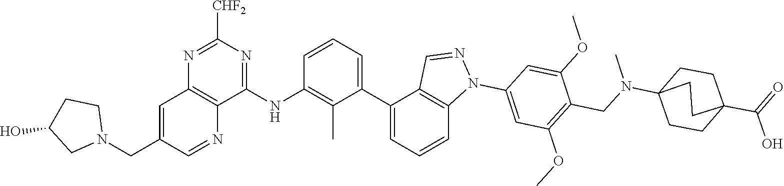 [SMILES: COc1cc(-n2ncc3c(-c4cccc(Nc5nc(C(F)F)nc6cc(CN7CC[C@@H](O)C7)cnc56)c4C)cccc32)cc(OC)c1CN(C)C12CCC(C(=O)O)(CC1)CC2]

### Step 1: tert-butyl (3-bromo-2-methylphenyl)(2-(difluoromethyl)-7-formylpyrido[3,2-d]pyrimidin-4-yl)carbamate

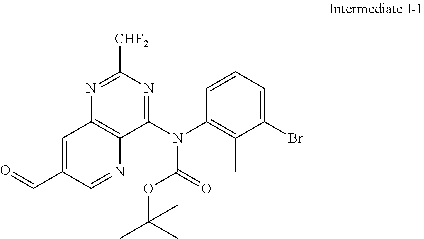 [SMILES: Cc1c(Br)cccc1N(C(=O)OC(C)(C)C)c1nc(C(F)F)nc2cc(C=O)cnc12]

A solution of di-tert-butyldicarbonate (500 mg, 2.29 mmol) in DCM (10 mL) was added to a mixture of Intermediate 1 (600 mg, 1.53 mmol), triethylamine (0.64 mL, 4.59 mmol), and 4-dimethylaminopyridine (0.02 g, 0.16 mmol) in DCM (10 mL). After the reaction was stirred at room temperature for 2 h, water was added into the solution. The resulting mixture was extracted three times with DCM and the organic layer was collected, washed with brine, dried over anhydrous Na₂SO₄, filtered, and evaporated to dryness. The crude mixture was purified by column chromatography (silica gel, gradient elution, 50% EtOAc/Hexanes) to provide the title compound.

### Step 2: (R)-tert-butyl (3-bromo-2-methylphenyl)(2-(difluoromethyl)-7-((3-hydroxypyrrolidin-1-yl)methyl)pyrido[3,2-d]pyrimidin-4-yl)carbamate

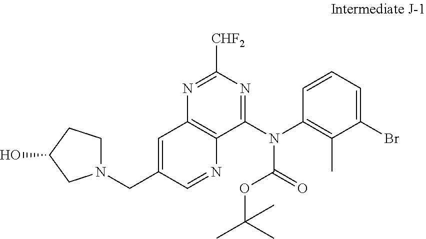 [SMILES: Cc1c(Br)cccc1N(C(=O)OC(C)(C)C)c1nc(C(F)F)nc2cc(CN3CC[C@@H](O)C3)cnc12]

This intermediate was prepared by using similar procedure described in Intermediate J, replacing Intermediate 1 with Intermediate 1-1. The crude mixture was purified by column chromatography (silica gel, gradient elution, 0 to 10% MeOH/DCM) to provide the title compound.

### Step 3: (R)-tert-butyl (2-(difluoromethyl)-7-((3-hydroxypyrrolidin-1-yl)methyl)pyrido[3,2-d]pyrimidin-4-yl)(3-(1-(4-formyl-3,5-dimethoxyphenyl)-1H-indazol-4-yl)-2-methylphenyl)carbamate

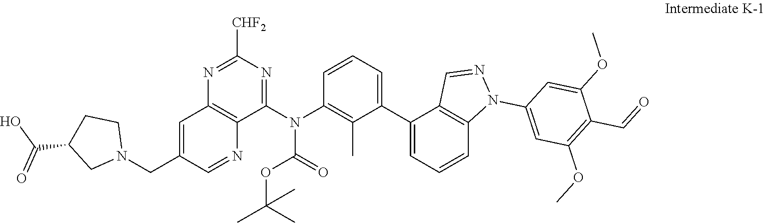 [SMILES: COc1cc(-n2ncc3c(-c4cccc(N(C(=O)OC(C)(C)C)c5nc(C(F)F)nc6cc(CN7CC[C@@H](C(=O)O)C7)cnc56)c4C)cccc32)cc(OC)c1C=O]

This intermediate was prepared by using similar procedure described in Intermediate K, replacing Intermediate J with Intermediate J-1. The crude mixture was purified by column chromatography (silica gel, gradient elution, 0 to 5% MeOH/DCM) to provide the title compound.

Step 4: (R)-4-((4-(4-(3-((tert-butoxycarbonyl)(2-(difluoromethyl)-7-((3-hydroxypyrrolidin-1-yl)methyl)pyrido[3,2-d]pyrimidin-4-yl)amino)-2-methylphenyl)-1H-indazol-1-yl)-2,6-dimethoxybenzyl)(methyl)amino)bicyclo[2.2.2]octane-1-carboxylic acid

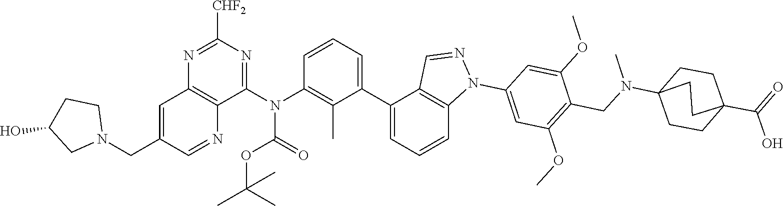 [SMILES: COc1cc(-n2ncc3c(-c4cccc(N(C(=O)OC(C)(C)C)c5nc(C(F)F)nc6cc(CN7CC[C@@H](O)C7)cnc56)c4C)cccc32)cc(OC)c1CN(C)C12CCC(C(=O)O)(CC1)CC2]

NEt₃ was added to a mixture of 4-aminobicyclo[2.2.2]octane-1-carboxylic acid hydrochloride (17.0 mg, 0.08 mmol) in 2 mL MeOH until the pH was around 7. Then, the mixture was transferred to a solution of Intermediate K-1 (30.0 mg, 0.04 mmol) in 2 mL DCM at room temperature. After that, acetic acid was added until the pH was around 4. After the reaction was stirred for overnight, sodium triacetoxyborohydride (30.0 mg, 0.14 mmol) was added. After the reaction was completed, 37% formaldehyde solution (1 mL) and sodium cyanoborohydride (10.0 mg, 0.16 mmol) were added, respectively. The reaction was stirred for 1 hour and evaporated to dryness. The crude mixture was purified by Sephadex column chromatography (100% MeOH) to provide the title compound.

Step 5: (R)-4-((4-(4-(3-((2-(difluoromethyl)-7-((3-hydroxypyrrolidin-1-yl)methyl)pyrido[3,2-d]pyrimidin-4-yl)amino)-2-methylphenyl)-1H-indazol-1-yl)-2,6-dimethoxybenzyl)(methyl)amino)bicyclo[2.2.2]octane-1-carboxylic acid

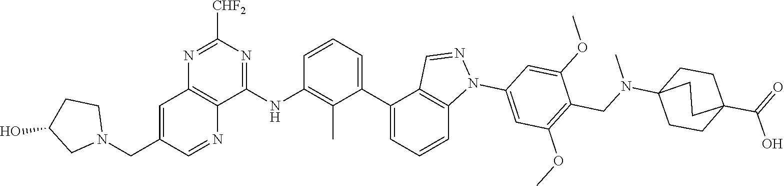 [SMILES: COc1cc(-n2ncc3c(-c4cccc(Nc5nc(C(F)F)nc6cc(CN7CC[C@@H](O)C7)cnc56)c4C)cccc32)cc(OC)c1CN(C)C12CCC(C(=O)O)(CC1)CC2]

A solution of TFA/DCM (1:4, 2 mL) was added to (R)-4-((4-(4-(3-((tert-butoxycarbonyl)(2-(difluoromethyl)-7-((3-hydroxypyrrolidin-1-yl)methyl)pyrido[3,2-d]pyrimidin-4-yl)amino)-2-methylphenyl)-1H-indazol-1-yl)-2,6-dimethoxybenzyl)(methyl)amino)bicyclo[2.2.2]octane-1-carboxylic acid (40.0 mg, 0.056 mmol). The reaction was stirred for 1 hour, evaporated to dryness, and purified by column chromatography (silica gel, gradient elution, 50 to 75% MeOH/DCM) to provide the title compound (4.48 mg, 95% purity by UV). LCMS Method G, Rt=10.045 min, m/z=833.4 [M+H]⁺, LCMS Method D, Rt=3.778 min, m/z=833.3944 [M+H]⁺, exact mass: 832.3872. ¹H NMR (500 MHz, MeOD-d₄) δ ppm 8.98 (s, 1H), 8.18 (s, 1H), 8.04 (s, 1H), 7.93 (d, J=8.37 Hz, 1H), 7.90 (dd, J=1.72, 8.83 Hz, 1H), 7.63 (ddd, J=2.53, 7.09, 8.61 Hz, 1H), 7.44 (t, J=7.79 Hz, 1H), 7.31 (d, J=7.62 Hz, 1H), 7.24 (dd, J=2.41, 7.03 Hz, 1H), 7.12 (d, J=6.15 Hz, 1H), 7.12 (s, 1H), 6.59 (t, J=54.84 Hz, 1H), 3.99 (s, 3H), 3.98 (s, 3H), 3.99-3.98 (m, 2H), 3.94 (dd, J=1.37, 13.79 Hz, 1H), 3.89 (dd, J=1.46, 13.67 Hz, 1H), 2.86-2.82 (m, 2H), 2.60-2.52 (m, 2H), 2.30-2.26 (m, 2H), 2.22-2.15 (m, 1H), 2.17 (s, 6H), 1.84-1.81 (m, 3H), 1.78-1.72 (m, 1H), 1.64-1.58 (m, 4H), 1.32-1.27 (m, 3H), 0.91-0.84 (m, 1H).

### Example 167: Biological Activity of the Compounds of the Present Disclosure

The biological activity of the compounds of the present disclosure was determined utilizing the assay described herein.

### PD-1′PD-L1 Protein-Protein Interaction Assay

Amplified Luminescent Proximity Homogeneous Assay (ALPHA) platform was used to evaluate the ability of the compounds to block the interaction between PD-1 and PD-L1. The assays were performed in half volume white 96-well plate in a final volume of 40 μl. Compounds at various concentrations were preincubated with 2 nM His tagged recombinant human PD-L1 protein and 0.6 nM recombinant PD-1 protein with Fc-tag (both reagents were purchased from AcroBiosystems) for 40 minutes. After the preincubation, 20 μg/mL of Alphascreen Ni chelate donor beads (PerkinElmer, USA) and Protein A acceptor beads (PerkinElmer, USA) were added into the well and incubated under reduced light at 25° C. for 120 minutes. The signal was measured with the EnSight Multimode Plate Reader. IC50 values were calculated from the four-parameter logistic curve fit.

### PD-1/PD-L1 Blockage Cell-Based Bioassay

Compounds were tested in a bioluminescent cell-based assay for PD-1/PD-L1 interaction blocking. Jurkat T cells expressing human PD-1 and luciferase gene reporter driven by TCR-mediated NFAT response element (Jurkat NFAT) were co-cultured with CHO-kl cells expressing human PD-L1 and surface-bound TCR activator (CHO-PDL1). Blocking of PD-1/PD-L1 interaction prevents the inhibitory signal from PD-1 and increases NFAT-mediated luminescence. CHO-PDL1 cells (20,000 cells/well) were seeded overnight. Compounds were added on CHO-PDL1 and incubated for 2 h at 37° C. Jurkat NFAT cells (20,000 cells/well) were diluted in RPMI assay medium (RPMI1640 with 1% FBS) and added into each well. After co-incubation for 6 h at 37° C., luminescence was determined by adding Bio-Glo™ Luciferase reagent (Promega) and measured with a luminescence plate reader. EC50 values were calculated from the four-parameter concentration-response curves.

Compounds of the present disclosure, as exemplified in the Examples, showed IC₅₀ values in the following ranges:
Alpha IC₅₀: A>100 nM; 1B=10-100 nM; C=1-10 nM; D<1 nM
NFAT IC₅₀: A>2000 mM B=201-2000 nM C≤00 nM

TABLE A

| Example | Alpha | NFAT |
|---|---|---|
| No. | IC₅₀, nM | IC₅₀, nM |
| 1 | B | A |
| 2 | A | — |
| 3 | A | — |
| 4 | B | A |
| 5 | A | — |
| 6 | A | — |
| 7 | A | — |
| 8 | A | — |
| 9 | A | — |
| 10 | A | — |
| 11 | B | A |
| 12 | A | — |
| 13 | C | A |
| 14 | A | — |
| 15 | B | A |
| 16 | A | — |
| 17 | A | — |
| 18 | A | — |
| 19 | A | — |
| 20 | A | — |
| 21 | A | — |
| 22 | A | — |
| 23 | B | — |
| 24 | A | — |
| 25 | A | — |
| 26 | A | — |
| 27 | C | C |
| 28 | C | A |
| 29 | D | C |
| 30 | B | A |
| 31 | C | B |
| 32 | C | A |
| 33 | B | A |
| 34 | C | A |
| 35 | C | B |
| 36 | B | A |
| 37 | C | B |
| 38 | B | A |
| 39 | D | B |
| 40 | D | C |
| 41 | C | B |
| 42 | D | C |
| 43 | D | C |
| 44 | D | C |
| 45 | D | A |
| 46 | C | A |
| 47 | D | C |
| 48 | B | A |
| 49 | D | B |
| 50 | B | A |
| 51 | D | C |
| 52 | D | C |
| 53 | D | B |
| 54 | B | A |
| 55 | C | B |
| 56 | A | — |
| 57 | A | — |
| 58 | A | — |
| 59 | A | — |
| 60 | A | — |
| 61 | A | — |
| 62 | A | — |
| 63 | A | — |
| 64 | C | A |
| 65 | C | B |
| 66 | D | C |
| 67 | C | A |
| 68 | D | C |
| 69 | D | C |
| 70 | C | A |
| 71 | D | B |
| 72 | D | C |
| 73 | D | B |
| 74 | D | C |
| 75 | D | B |
| 76 | D | C |
| 77 | D | B |
| 78 | D | C |
| 79 | D | C |
| 80 | A | — |
| 81 | B | A |
| 82 | C | B |
| 83 | C | B |
| 84 | D | B |
| 85 | B | A |
| 86 | A | — |
| 87 | C | A |
| 88 | C | B |
| 89 | D | C |
| 90 | C | B |
| 91 | C | B |
| 92 | D | C |
| 93 | C | A |
| 94 | D | C |
| 95 | C | B |
| 96 | C | B |
| 97 | D | C |
| 98 | C | B |
| 99 | D | C |
| 100 | D | C |
| 101 | D | C |
| 102 | C | C |
| 103 | C | C |
| 104 | C | B |
| 105 | D | C |
| 106 | D | B |
| 107 | D | C |
| 108 | D | C |
| 109 | C | C |
| 110 | D | B |
| 111 | D | B |
| 112 | D | C |
| 113 | D | C |
| 114 | C | B |
| 115 | D | C |
| 116 | D | C |
| 117 | D | C |
| 118 | D | A |
| 119 | D | B |
| 120 | D | B |
| 121 | D | B |
| 122 | C | B |
| 123 | D | C |
| 124 | D | B |
| 125 | D | C |
| 126 | D | C |
| 127 | D | C |
| 128 | D | C |
| 129 | D | C |
| 130 | D | C |
| 131 | C | B |
| 132 | D | C |
| 133 | C | C |
| 134 | D | C |
| 135 | D | C |
| 136 | D | B |
| 137 | D | B |
| 138 | C | B |
| 139 | D | C |
| 140 | C | B |
| 141 | D | C |
| 142 | D | C |
| 143 | D | C |
| 144 | D | C |
| 145 | C | B |
| 146 | D | B |
| 147 | C | B |
| 148 | D | C |
| 149 | D | C |
| 150 | D | C |
| 151 | C | C |
| 152 | D | C |
| 153 | D | C |
| 154 | D | C |
| 155 | D | C |
| 156 | D | C |
| 157 | D | C |
| 158 | D | C |
| 159 | D | C |
| 160 | D | C |
| 161 | D | C |
| 162 | D | C |
| 163 | D | B |
| 164 | D | C |
| 165 | D | C |
| 166 | D | C |

### EQUIVALENTS

The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference.

The foregoing description has been presented only for the purposes of illustration and is not intended to limit the disclosure to the precise form disclosed, but by the claims appended hereto.

1. A compound of Formula (I): 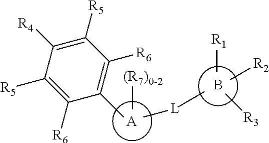
or a prodrug, solvate, or pharmaceutically acceptable salt thereof, wherein:
Ring A is 7- to 10-membered heterocyclyl or 7- to 10-membered heteroaryl;
Ring B is C₃-C₁₀ cycloalkyl, 3- to 10-membered heterocyclyl, C₆-C₁₀ aryl, or 5- to 10-membered heteroaryl;
L is absent, —C(O)—, or —CH₂—;
each R₁ and R₃ is independently H, halogen, —CN, C₁-C₆ alkyl, C₂-C₆ alkenyl, C₂-C₆ alkynyl, C₁-C₆ haloalkyl, —O—C₁-C₆alkyl, —NH—C₁-C₆alkyl, or C₃-C₇ cycloalkyl, wherein the alkyl, alkenyl, alkynyl, or cycloalkyl are optionally substituted with one or more R₁ₐ;
R₁ₐ is C₆-C₁₀ aryl optionally substituted with one or more halogen, —CN, or —OH;
R₂ is H, —(CH₂)ₙ—N(R₂ₐ)(R_{2b}), —(CH₂)ₙ—OH, —(CH₂)˜—O(C₁-C₆ alkyl), —(CH₂)ₙ—O(C₆-C₁₀ aryl), C₃-C₇ cycloalkyl, 3- to 10-membered heterocyclyl, C₆-C₁₀ aryl, or 5- to 10-membered heteroaryl, wherein the cycloalkyl, heterocyclyl, aryl, and heteroaryl are optionally substituted with one or more R_{2b′};
R₂ₐ is H or C₁-C₆ alkyl;
R_{2b} is C₁-C₆ alkyl, C₂-C₆ alkenyl, C₂-C₆ alkynyl, C₁-C₆ haloalkyl, or C₃-C₇ cycloalkyl, wherein the alkyl, alkenyl, alkynyl, or cycloalkyl are optionally substituted with one or more R_{2b′}, or R_{2b} is 3- to 10-membered heterocyclyl optionally substituted with one or more oxo or —(C₁-C₆ alkyl)-OH,
or R₂ₐ and R_{2b} come together to form a 3- to 10-membered heterocyclyl optionally substituted with one or more R_{2b′};
R_{2b′} is oxo, halogen, C₁-C₆ alkyl, C₂-C₆ alkenyl, C₂-C₆ alkynyl, C₁-C₆ haloalkyl, —(CH₂)ₘ—OR_{2b″}, —(CH₂)ₘ—N(R_{2b′})(R_{2b′″}), —(CH₂)ₙ—C(O)OR_{2b′}, —C(O)R_{2b″}, —C(O)N(R_{2b″})(R_{2b}-j), —N(R_{2b′})C(O)R_{2b′}, C₃-C₇ cycloalkyl, 3- to 10-membered heterocyclyl, or 5- to 10-membered heteroaryl, wherein the alkyl, alkenyl, alkynyl, cycloalkyl, and heterocyclyl are optionally substituted with one or more R_{2b″};
R_{2b″} is H, oxo, —CN, —OH, C₁-C₆ alkyl, C₂-C₆ alkenyl, C₂-C₆ alkynyl, or C₁-C₆ haloalkyl, wherein the alkyl, alkenyl, and alkynyl are optionally substituted with 3- to 10-membered heterocyclyl, wherein the heterocyclyl is optionally substituted with oxo;
R_{2b″} is H, C₁-C₆ alkyl optionally substituted with one or more —C(O)OH, —OH or —NH₂, C₂-C₆ alkenyl, C₂-C₆ alkynyl, C₃-C₇ cycloalkyl, or —C(O)C₁-C₆ alkyl;
each R₄ or R₅ is independently H, —O—C₁-C₆alkyl, —NH—C₁-C₆alkyl, —NH-(5- to 10-membered heteroaryl), —NH—(C₆-C₁₀ aryl), —NH—C(O)R₅ₐ, C₁-C₆ alkyl, C₂-C₆ alkenyl, C₂-C₆ alkynyl, C₁-C₆ haloalkyl, C₆-C₁₀ aryl, or 5- to 10-membered heteroaryl, wherein the alkyl, alkenyl, alkynyl, aryl, or heteroaryl are optionally substituted with one or more R₅ₐ₁, or R₄ and R₅ come together to form a C₄-C₇ cycloalkyl or 4- to 10-membered heterocyclyl;
R₅ₐ is C₃-C₇ cycloalkyl, 3- to 10-membered heterocyclyl, C₁-C₁₀ aryl, or 5- to 10-membered heteroaryl, wherein the cycloalkyl, heterocyclyl, aryl, and heteroaryl are optionally substituted with one or more R₅ₐ₁;
R₅ₐ₁ is halogen, —CN, —(CH₂)ₚ—N(R_{5a1′})(R_{5b1′}), —O—C₁-C₆alkyl, —(CH₂)ₚ—C₃-C₇ cycloalkyl wherein the cycloalkyl is optionally substituted with one or more —C(O)OH, —OH or —NH₂, C₁-C₆ alkyl optionally substituted with one or more halogen, —C(O)OH, —OH, or —NH₂, or 3- to 10-membered heterocyclyl optionally substituted with one or more —(C₁-C₆ alkyl)-OH;
R_{5b1′} is H or C₁-C₆ alkyl;
R_{5a1″} is H, C₁-C₆ alkyl, C₂-C₆ alkenyl, C₂-C₆ alkynyl, C₁-C₆ haloalkyl, C₃-C₈ cycloalkyl, 3- to 10-membered heterocyclyl, C₆-C₁₀ aryl, or 5- to 10-membered heteroaryl, wherein the alkyl, alkenyl, alkynyl, cycloalkyl, heterocyclyl, aryl, and heteroaryl are optionally substituted with one or more R_{5a1″},
or R_{5a1′} and R_{5b1′} come together to form a 3- to 10-membered heterocyclyl optionally substituted with one or more R_{5a1″};
R_{5a1′} is oxo, —CN, —OH, C₁-C₆ alkyl optionally substituted with one or more —C(O)OH, —OH or —NH₂, C₂-C₆ alkenyl, C₂-C₆ alkynyl, C₁-C₆ haloalkyl, —C(O)OH, —C(O)C₁-C₆ alkyl, —C(O)O—C, —C₆ alkyl, —C(O)NH₂, —C(O)NH—C₁-C₆ alkyl, —C(O)NH—C₃-C₁₀ cycloalkyl, —NH—C₁-C₆ alkyl-R_{5a1″}, —(CH₂)_{q}—(C₇-C₇ cycloalkyl), or —(CH₂)_{q}-(3- to 10-membered heterocyclyl), wherein the cycloalkyl and heterocyclyl are optionally substituted with one or more oxo, C₁-C₆ alkyl, C₂-C₆ alkenyl, C₂-C₆ alkynyl, —C(O)OH, or —OH;
R_{5a1′″} is 3- to 10-membered heterocyclyl optionally substituted with oxo;
each R₆ is independently H, halogen, —CN, C₁-C₆ alkyl, C₂-C₆ alkenyl, C₂-C₆ alkynyl, C₁-C₆ haloalkyl, —O—C₁-C₆ alkyl, —NH—C₁-C₆alkyl, or C₃-C₇ cycloalkyl;
R₇ is C₁-C₆ alkyl, C₂-C₆ alkenyl, C₂-C₆ alkynyl, or C₁-C₆ haloalkyl;
m is 0, 1, 2, 3, or 4;
n is 0, 1, 2, 3, or 4;
p is 0, 1, 2, 3, or 4; and
q is 0, 1, 2, 3, or 4,
wherein at least one of R₄, R₅, or R₆ is not H; and
provided that:
(ii) when Ring B is an optionally substituted 9-membered heterocyclyl and R₅ₐ₁ and R_{5b′} come together to form a 6-membered heterocyclyl, then R₂ is not —(CH₂)₂—OH; or
(iii) when R₄ and R₅ come together to form a 6-membered heterocyclyl and Ring B is 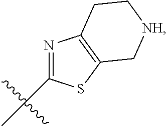 [SMILES: *c1nc2c(s1)CNCC2]
then Ring B is not substituted with —CH₃ or —CH₂CH₂—OH; or
(iv) when R₅ is —NH—C(O)R₅ₐ, R₅ₐ is not a substituted 5-membered heteroaryl.

2. The compound of claim 1, wherein:
Ring A is 7- to 10-membered heterocyclyl or 7- to 10-membered heteroaryl;
Ring B is C₃-C₁₀ cycloalkyl, 3- to 10-membered heterocyclyl, C₆-C₁₀ aryl, or 5- to 10-membered heteroaryl;
L is absent, —C(O)—, or —CH₂—;
each R₁ and R₃ is independently H, halogen, —CN, C₁-C₆ alkyl, C₂-C₆ alkenyl, C₂-C₆ alkynyl, C₁-C₆ haloalkyl, —O—C₁-C₆ alkyl, —NH—C₁-C₆ alkyl, or C₃-C₇ cycloalkyl, wherein the alkyl, alkenyl, alkynyl, or cycloalkyl is optionally substituted with one or more R₁ₐ;
R₁ₐ is C₆-C₁₀ aryl optionally substituted with one or more halogen, —CN, or —OH;
R₂ is H, —(CH₂)ₙ—N(R₂ₐ)(R_{2b}), —(CH₂)ₙ—OH, —(CH₂)ₙ—O(C₁-C₆ alkyl), —(CH₂)ₙ—O(C₆-C₁₀ aryl), C₃-C₇ cycloalkyl, 3- to 10-membered heterocyclyl, C₆-C₁₀ aryl, or 5- to 10-membered heteroaryl, wherein the cycloalkyl, heterocyclyl, aryl, or heteroaryl is optionally substituted with one or more R_{2b′};
R₂ₐ is H;
R_{2b} is C₁-C₆ alkyl, C₂-C₆ alkenyl, C₂-C₆ alkynyl, C₁-C₆ haloalkyl, or C₃-C₇ cycloalkyl, wherein the alkyl, alkenyl, alkynyl, or cycloalkyl is optionally substituted with one or more R_{2b′}, or R_{2b} is 3- to 10-membered heterocyclyl optionally substituted with one or more oxo or —(C₁-C₆ alkyl)-OH,
or R₂ₐ and R_{2b} come together to form a 3- to 10-membered heterocyclyl optionally substituted with one or more R_{2b′};
R_{2b″} is oxo, halogen, C₁-C₆ alkyl, C₂-C₆ alkenyl, C₂-C₆ alkynyl, C₁-C₆ haloalkyl, —(CH₂)ₘ—OR_{2b′}, —(CH₂)ₘ—N(R_{2b′})(R_{2b″}), —(CH₂)ₘ—C(O)OR_{2b′}, —C(O)R_{2b″}, —C(O)N(R_{2b′})(R_{2b′″}), —N(R_{2b′})C(O)R_{2b″}, C₃-C₇ cycloalkyl, or 3- to 10-membered heterocyclyl, wherein the alkyl, alkenyl, alkynyl, cycloalkyl, or heterocyclyl is optionally substituted with one or more R_{2b″};
R_{2b″} is H, oxo, —CN, —OH, C₁-C₆ alkyl, C₂-C₆ alkenyl, C₂-C₆ alkynyl, or C₁-C₆ haloalkyl, wherein the alkyl, alkenyl, or alkynyl is optionally substituted with 3- to 10-membered heterocyclyl, wherein the heterocyclyl is optionally substituted with oxo;
R_{2b′″} is H, C₁-C₆ alkyl optionally substituted with one or more —C(O)OH, —OH or —NH₂, C₂-C₆ alkenyl, C₂-C₆ alkynyl, C₃-C₇ cycloalkyl, or —C(O)C₁-C₆ alkyl;
each R₄ or R₅ is independently H, —O—C₁-C₆alkyl, —NH—C₁-C₆alkyl, —NH-(5- to 10-membered heteroaryl), —NH—(C₆-C₁₀ aryl), —NH—C(O)R₅ₐ, C₁-C₆ alkyl, C₂-C₆ alkenyl, C₂-C₆ alkynyl, C₁-C₆ haloalkyl, C₆-C₁₀ aryl, or 5- to 10-membered heteroaryl, wherein the alkyl, alkenyl, alkynyl, aryl, or heteroaryl is optionally substituted with one or more R₅ₐ₁, or R₄ and R₅ come together to form a C₄-C₇ cycloalkyl or 4- to 10-membered heterocyclyl;
R₅ₐ is C₃-C₇ cycloalkyl, 3- to 10-membered heterocyclyl, C₆-C₁₀ aryl, or 5- to 10-membered heteroaryl, wherein the cycloalkyl, heterocyclyl, aryl, or heteroaryl is optionally substituted with one or more R₅ₐ₁;
R₅ₐ₁ is halogen, —CN, —(CH₂)ₚ—N(R_{5a1′})(R_{5b1′}), —O—C₁-C₆alkyl, —(CH₂)ₚ—C₃-C₇ cycloalkyl wherein the cycloalkyl is optionally substituted with one or more —C(O)OH, —OH or —NH₂, C₁-C₆ alkyl optionally substituted with one or more halogen, —COOH, —OH, or —NH₂, or 3- to 10-membered heterocyclyl optionally substituted with one or more —(C₁-C₆ alkyl)-OH;
R_{5b1′} is H;
R_{5a1′} is H, C₁-C₆ alkyl, C₂-C₆ alkenyl, C₂-C₆ alkynyl, C₁-C₆ haloalkyl, C₃-C₇ cycloalkyl, 3- to 10-membered heterocyclyl, C₆-C₁₀ aryl, or 5- to 10-membered heteroaryl, wherein the alkyl, alkenyl, alkynyl, cycloalkyl, heterocyclyl, aryl, or heteroaryl is optionally substituted with one or more R_{5a1″},
or R_{5a1′} and R_{5a1″} come together to form a 3- to 10-membered heterocyclyl optionally substituted with one or more R_{5a1″};
R_{5a1″} is oxo, —CN, —OH, C₁-C₆ alkyl optionally substituted with one or more —C(O)OH, —OH or —NH₂, C₂-C₆ alkenyl, C₂-C₆ alkynyl, C₁-C₆ haloalkyl, —C(O)OH, —C(O)C₁-C₆ alkyl, —C(O)O—C, —C₆ alkyl, —C(O)NH₂, —C(O)NH—C₁-C₆ alkyl, —C(O)NH—C₃-C₁₀ cycloalkyl, —NH—C₁-C₆ alkyl-R_{5a1″}, —(CH₂)_{q}—(C₂-C₇ cycloalkyl), or —(CH₂)_{q}-(3- to 10-membered heterocyclyl), wherein the cycloalkyl or heterocyclyl is optionally substituted with one or more oxo, C₁-C₆ alkyl, C₂-C₆ alkenyl, C₂-C₆ alkynyl, —C(O)OH, or —OH;
R_{5a1′″} is 3- to 10-membered heterocyclyl optionally substituted with oxo;
each R₆ is independently H, halogen, —CN, C₁-C₆ alkyl, C₂-C₆ alkenyl, C₂-C₆ alkynyl, C₁-C₆ haloalkyl, —O—C₁-C₆alkyl, —NH—C₁-C₆alkyl, or C₃-C₇ cycloalkyl;
R₇ is C₁-C₆ alkyl, C₂-C₆ alkenyl, C₂-C₆ alkynyl, or C₁-C₆ haloalkyl;
m is 0, 1, 2, 3, or 4;
n is 0, 1, 2, 3, or 4;
p is 0, 1, 2, 3, or 4; and
q is 0, 1, 2, 3, or 4,
wherein at least one of R₄, R₅, or R₆ is not H; and
provided that:
(ii) when Ring B is an optionally substituted 9-membered heterocyclyl and R₅ₐ₁ and R_{5a1′} come together to form a 6-membered heterocyclyl, then R₂ is not —(CH₂)₂—OH; or
(iii) when R₄ and R₅ come together to form a 6-membered heterocyclyl and Ring B is 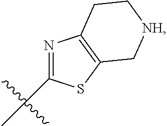 [SMILES: *c1nc2c(s1)CNCC2]
then Ring B is not substituted with —CH₃ or —CH₂CH₂—OH; or
(iv) when R₅ is —NH—C(O)R₅ₐ, R₅ₐ is not a substituted 5-membered heteroaryl.

3. The compound of claim 1, wherein Ring A is 9-membered heterocyclyl.

4. The compound of claim 1, wherein Ring A is 9-membered heteroaryl.

5. The compound of claim 1, wherein Ring A is indole or indazole.

6. The compound of any one of the preceding claims, wherein Ring B is 3- to 10-membered heterocyclyl, C₆-C₁₀ aryl, or 5- to 10-membered heteroaryl.

7. The compound of any one of the preceding claims, wherein L is absent.

8. The compound of any one of claim 1-5, wherein L is —C(O)— or —CH₂—.

9. The compound of any one of the preceding claims, wherein each R₁ and R₃ is independently H, C₁-C₆ alkyl, or —O—C₁-C₆ alkyl, wherein the alkyl is optionally substituted with one or more R₁ₐ.

10. The compound of any one of the preceding claims, wherein R₁ₐ is C₆-C₁₀ aryl optionally substituted with one or more —CN.

11. The compound of any one of the preceding claims, wherein R₁ₐ is C₆-C₁₀ aryl substituted with one or more halogen, —CN, or —OH.

12. The compound of any one of the preceding claims, wherein R₂ is H, —(CH₂)ₙ—N(R₂ₐ)(R_{2b}), C₃-C₇ cycloalkyl, 3- to 10-membered heterocyclyl, C₆-C₁₀ aryl, or 5- to 10-membered heteroaryl, wherein the cycloalkyl, heterocyclyl, aryl, or heteroaryl is optionally substituted with one or more R_{2b′}.

13. The compound of any one of the preceding claims, wherein R_{2b} is C₁-C₆ alkyl or C₃-C₇ cycloalkyl, wherein the alkyl or cycloalkyl is optionally substituted with one or more R_{2b′}.

14. The compound of any one of claims 1-12, wherein R_{2b} is 3- to 10-membered heterocyclyl substituted with one or more oxo or —(C₁-C₆ alkyl)-OH.

15. The compound of any one of claims 1-12, wherein R₂ₐ and R_{2b} come together to form a 3- to 10-membered heterocyclyl substituted with one or more R_{2b′}.

16. The compound of any one of the preceding claims, wherein R_{2b′} is C₁-C₆ alkyl, —(CH₂)ₘ—OR_{2b′}, —(CH₂)ₘ—N(R_{2b′})(R_{2b″}), —C(O)OR_{2b′}, —C(O)R_{2b′}, —C(O)N(R_{2b″})(R_{2b′″}), —N(R_{2b′})C(O)R_{2b′}, C₃-C₇ cycloalkyl, or 3- to 10-membered heterocyclyl, wherein the alkyl, cycloalkyl, or heterocyclyl is optionally substituted with one or more R_{2b′}.

17. The compound of any one of the preceding claims, wherein R_{2b″} is H, oxo, —CN, —OH, C₁-C₆ alkyl, wherein the alkyl is optionally substituted with 3- to 10-membered heterocyclyl, wherein the heterocyclyl is optionally substituted with oxo.

18. The compound of any one of the preceding claims, wherein R_{2b″} is H, C₁-C₆ alkyl, C₃-C₇ cycloalkyl, or —C(O)C₁-C₆ alkyl.

19. The compound of any one of the preceding claims, wherein each R₄ or R₅ is independently H, —O—C₁-C₆ alkyl, —NH—C(O)R₅ₐ, C₁-C₆ alkyl, C₆-C₁₀ aryl, or 5- to 10-membered heteroaryl, wherein the alkyl, aryl, or heteroaryl is optionally substituted with one or more R₅ₐ₁.

20. The compound of any one of claims 1-18, wherein R₄ and R₅ come together to form a C₄-C₇ cycloalkyl.

21. The compound of any one of the preceding claims, wherein R₅ₐ₁ is —O—C₁-C₆ alkyl, C₃-C₇ cycloalkyl, C₁-C₆ alkyl optionally substituted with one or more —OH or —NH₂, or 3- to 10-membered heterocyclyl optionally substituted with one or more —(C₁-C₆ alkyl)-OH.

22. The compound of any one of the preceding claims, wherein R_{5a1′} is H, C₁-C₆ alkyl, C₁-C₆ haloalkyl, C₃-C₇ cycloalkyl, 3- to 10-membered heterocyclyl, C₆-C₁₀ aryl, or 5- to 10-membered heteroaryl, wherein the alkyl, cycloalkyl, heterocyclyl, aryl, or heteroaryl is optionally substituted with one or more R_{5a1″}.

23. The compound of any one of claims 1-21, wherein R_{5a1′} and R_{5b1′} come together to form a 3- to 10-membered heterocyclyl substituted with one or more R_{5a1″}.

24. The compound of any one of the preceding claims, wherein R_{5a1″} is oxo, —OH, C₁-C₆ alkyl, —C(O)OH, —C(O)C₁-C₆ alkyl, —C(O)O—C₁-C₆ alkyl, —C(O)NH—C₁-C₆ alkyl, —C(O)NH—C₃-C₁₀ cycloalkyl, —NH—C₁-C₆ alkyl-R_{5a1′}, —(CH₂)_{q}—(C₃-C₇ cycloalkyl), or —(CH₂)_{q}-(3- to 10-membered heterocyclyl), wherein the cycloalkyl or heterocyclyl is optionally substituted with one or more oxo, C₁-C₆ alkyl, —C(O)OH, or —OH.

25. The compound of any one of the preceding claims, wherein R_{5a1′″} is 3- to 10-membered heterocyclyl substituted with oxo.

26. The compound of any one of the preceding claims, wherein each R₆ is independently H, halogen, C₁-C₆ alkyl, —O—C₁-C₆ alkyl, —NH—C₁-C₆alkyl, or C₃-C₇ cycloalkyl.

27. The compound of any one of the preceding claims, wherein R₇ is C₁-C₆ alkyl.

28. The compound of any one of the preceding claims, wherein m is 0 or 1.

29. The compound of any one of the preceding claims, wherein p is 0 or 1.

30. The compound of any one of the preceding claims, wherein the compound is of Formula (I-a) or (I-b): 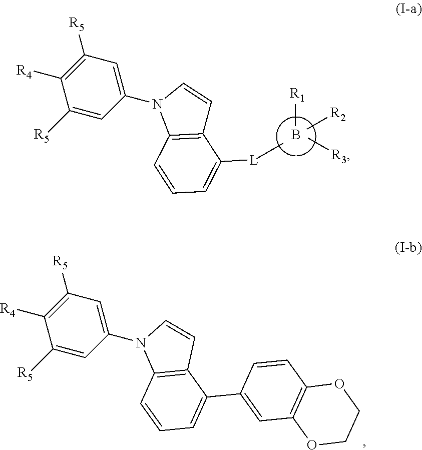
or a prodrug, solvate, or pharmaceutically acceptable salt thereof.

31. The compound of any one of the preceding claims, wherein the compound is of Formula (I-c) or (I-d): 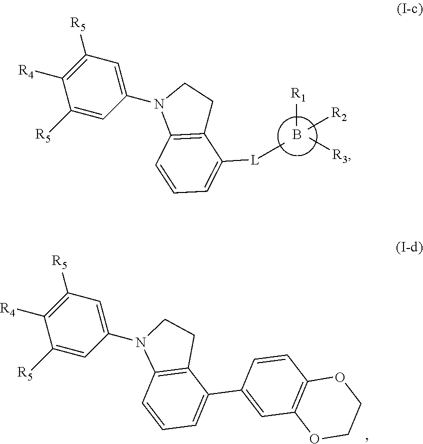
or a prodrug, solvate, or pharmaceutically acceptable salt thereof.

32. The compound of any one of the preceding claims, wherein the compound is of Formula (I-e) or (I-f): 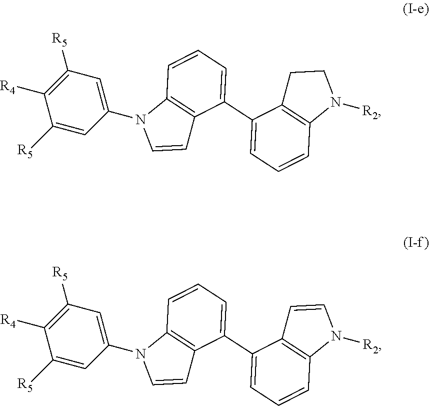
or a prodrug, solvate, or pharmaceutically acceptable salt thereof.

33. The compound of any one of the preceding claims, wherein the compound is of Formula (I-g): 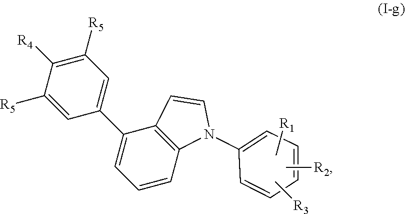
or a prodrug, solvate, or pharmaceutically acceptable salt thereof.

34. The compound of any one of the preceding claims, wherein the compound is of Formula (I-h) or (I-i): 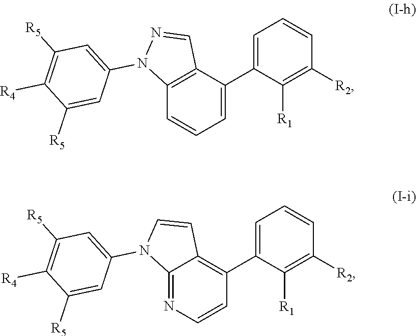
or a prodrug, solvate, or pharmaceutically acceptable salt thereof.

35. The compound of any one of the preceding claims, wherein the compound is of Formula (I-k): 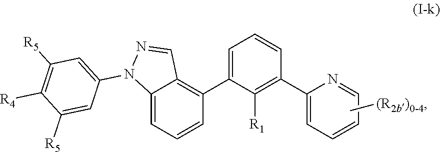
or a prodrug, solvate, or pharmaceutically acceptable salt thereof.

36. The compound of any one of the preceding claims, being selected from Compound Nos. 1-328 and prodrugs and pharmaceutically acceptable salts thereof.

37. The compound of any one of the preceding claims, being selected from Compound Nos. 1-328 and pharmaceutically acceptable salts thereof.

38. The compound of any one of the preceding claims, being selected from Compound Nos. 1-328.

39. A compound obtainable by, or obtained by, a method described herein;
optionally, the method comprises one or more steps described in Schemes 1-8.

40. A pharmaceutical composition comprising the compound of any one of claims 1-39 or a pharmaceutically acceptable salt thereof, and a pharmaceutically acceptable diluent or carrier.

41. The pharmaceutical composition of claim 40, wherein the compound is selected from Compound Nos. 1-328.

42. A method of modulating PD-1 activity, PD-L1 activity, and/or the PD-1/PD-L1 interaction, comprising contacting a cell or protein with an effective amount of the compound of any one of claims 1-39 or a pharmaceutically acceptable salt thereof; optionally the activity and/or interaction is in vitro or in vivo.

43. A method of treating or preventing a disease or disorder in a subject in need thereof, comprising administering to the subject the compound of any one of claims 1-39 or a pharmaceutically acceptable salt thereof, or the pharmaceutical composition of claim 40 or claim 41.

44. The compound of any one of claims 1-39, or the pharmaceutical composition of claim 40 or claim 41, for use in modulating PPD-1 activity, PD-L1 activity, and/or the PD-1/PD-L1 interaction; optionally, the activity and/or interaction is in vitro or in vivo.

45. The compound of any one of claims 1-39, or the pharmaceutical composition of claim 40 or claim 41, for use in treating or preventing a disease or disorder.

46. Use of the compound of any one of claims 1-39 or a pharmaceutically acceptable salt thereof in the manufacture of a medicament for modulating PD-1 activity, PD-L1 activity, and/or the PD-1/PD-L1 interaction; optionally, the activity and/or interaction is in vitro or in vivo.

47. Use of the compound of any one of claims 1-39 or a pharmaceutically acceptable salt thereof in the manufacture of a medicament for treating or preventing a disease or disorder.

48. The method, compound, pharmaceutical composition, or use of any one of the preceding claims, wherein the disease or disorder is associated with an implicated PD-1 activity, PD-L1 activity, and/or the PD-1/PD-L1 interaction.

49. The method, compound, pharmaceutical composition, or use of any one of the preceding claims, wherein the disease or disorder is cancer.

50. The method, compound, pharmaceutical composition, or use of any one of the preceding claims, in combination with at least one additional anti-cancer agent or therapy.

51. The method, compound, pharmaceutical composition, or use of claim 50, wherein the anti-cancer agent or therapy is nivolumab, pembrolizumab, atezolizumab, ipilimumab, chemotherapy, radiation therapy, or resection therapy.
